SEQUENCE LISTING (310 sequences)

SEQ ID NO: 1 - protein, 117 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 9 aa, synthetic construct
GSFSGYYWS

SEQ ID NO: 3 - protein, 16 aa, synthetic construct
EIDHSGSTNYNPSLKS

SEQ ID NO: 4 - protein, 11 aa, synthetic construct
ARARGPWSFDP

SEQ ID NO: 5 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 6 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 7 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 8 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 9 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 10 - protein, 117 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 11 - protein, 107 aa, synthetic construct
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 12 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 13 - protein, 107 aa, synthetic construct
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 14 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 15 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 16 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 17 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 18 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 19 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 20 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 21 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 22 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 23 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 24 - protein, 107 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 25 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 26 - protein, 125 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 27 - protein, 9 aa, synthetic construct
GTFSSYAIS

SEQ ID NO: 28 - protein, 5 aa, synthetic construct
SYAIS

SEQ ID NO: 29 - protein, 17 aa, synthetic construct
GIIPIFGTANYAQKFQG

SEQ ID NO: 30 - protein, 18 aa, synthetic construct
ARGDSSIRHAYYYYGMDV

SEQ ID NO: 31 - protein, 16 aa, synthetic construct
GDSSIRHAYYYYGMDV

SEQ ID NO: 32 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 33 - protein, 17 aa, synthetic construct
KSSQSVLYSSNNKNYLA

SEQ ID NO: 34 - protein, 7 aa, synthetic construct
WASTRES

SEQ ID NO: 35 - protein, 9 aa, synthetic construct
QQYYSTPIT

SEQ ID NO: 36 - protein, 121 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 37 - protein, 11 aa, synthetic construct
GSISSSSYYWG

SEQ ID NO: 38 - protein, 7 aa, synthetic construct
SSSYYWG

SEQ ID NO: 39 - protein, 16 aa, synthetic construct
SIYYSGSTYYNPSLKS

SEQ ID NO: 40 - protein, 13 aa, synthetic construct
ARGSDRFHPYFDY

SEQ ID NO: 41 - protein, 11 aa, synthetic construct
GSDRFHPYFDY

SEQ ID NO: 42 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 43 - protein, 11 aa, synthetic construct
RASQSVSRYLA

SEQ ID NO: 44 - protein, 7 aa, synthetic construct
DASNRAT

SEQ ID NO: 45 - protein, 9 aa, synthetic construct
QQFDTWPPT

SEQ ID NO: 46 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 47 - protein, 126 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 48 - protein, 19 aa, synthetic construct
ARRGRKASGSFYYYYGMDV

SEQ ID NO: 49 - protein, 113 aa, synthetic construct [sequence omitted]

SEQ ID NO: 50 - protein, 17 aa, synthetic construct
ESSQSLLNSGNQKNYLT

SEQ ID NO: 51 - protein, 9 aa, synthetic construct
QNDYSYPYT

SEQ ID NO: 52 - protein, 126 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 53 - protein, 9 aa, synthetic construct
YTFTSYYMH

SEQ ID NO: 54 - protein, 5 aa, synthetic construct
SYYMH

SEQ ID NO: 55 - protein, 17 aa, synthetic construct
IINPSGGSTSYAQKFQG

SEQ ID NO: 56 - protein, 19 aa, synthetic construct
ARGAPNYGDTTHDYYYMDV

SEQ ID NO: 57 - protein, 17 aa, synthetic construct
GAPNYGDTTHDYYYMDV

SEQ ID NO: 58 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 59 - protein, 11 aa, synthetic construct
RASQSVSSNLA

SEQ ID NO: 60 - protein, 7 aa, synthetic construct
GASTRAT

SEQ ID NO: 61 - protein, 9 aa, synthetic construct
QQYDDWPFT

SEQ ID NO: 62 - protein, 124 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 63 - protein, 9 aa, synthetic construct
YTFTGYYMH

SEQ ID NO: 64 - protein, 5 aa, synthetic construct
GYYMH

SEQ ID NO: 65 - protein, 17 aa, synthetic construct
WINPNSGGTNYAQKFQG

SEQ ID NO: 66 - protein, 17 aa, synthetic construct
ARDTGEYYDTDDHGMDV

SEQ ID NO: 67 - protein, 15 aa, synthetic construct
DTGEYYDTDDHGMDV

SEQ ID NO: 68 - protein, 107 aa, synthetic construct
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 69 - protein, 9 aa, synthetic construct
QQDDYWPPT

SEQ ID NO: 70 - protein, 121 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 71 - protein, 9 aa, synthetic construct
FTFSSYAMS

SEQ ID NO: 72 - protein, 17 aa, synthetic construct
AISGSGGSTYYADSVKG

SEQ ID NO: 73 - protein, 14 aa, synthetic construct
AKDGGYYDSGAGDY

SEQ ID NO: 74 - protein, 12 aa, synthetic construct
DGGYYDSGAGDY

SEQ ID NO: 75 - protein, 107 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 76 - protein, 11 aa, synthetic construct
RASQGIDSWLA

SEQ ID NO: 77 - protein, 7 aa, synthetic construct
AASSLQS

SEQ ID NO: 78 - protein, 9 aa, synthetic construct
QQGVSYPRT

SEQ ID NO: 79 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 80 - protein, 9 aa, synthetic construct
FTFSSYSMN

SEQ ID NO: 81 - protein, 5 aa, synthetic construct
SYSMN

SEQ ID NO: 82 - protein, 17 aa, synthetic construct
SISSSSSYIYYADSVKG

SEQ ID NO: 83 - protein, 15 aa, synthetic construct
ARGAPMGAAAGWFDP

SEQ ID NO: 84 - protein, 13 aa, synthetic construct
GAPMGAAAGWFDP

SEQ ID NO: 85 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 86 - protein, 11 aa, synthetic construct
RASQGISSWLA

SEQ ID NO: 87 - protein, 9 aa, synthetic construct
QQGVSFPRT

SEQ ID NO: 88 - protein, 249 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPMGAAAGWFDPWGQGTLVTVSS

SEQ ID NO: 89 - protein, 125 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 90 - protein, 18 aa, synthetic construct
AREGAGFAYGMDYYYMDV

SEQ ID NO: 91 - protein, 16 aa, synthetic construct
EGAGFAYGMDYYYMDV

SEQ ID NO: 92 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 93 - protein, 11 aa, synthetic construct
RASQSVSSYLA

SEQ ID NO: 94 - protein, 9 aa, synthetic construct
QQSDNWPFT

SEQ ID NO: 95 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 96 - protein, 15 aa, synthetic construct
ARGAPIGAAAGWFDP

SEQ ID NO: 97 - protein, 13 aa, synthetic construct
GAPIGAAAGWFDP

SEQ ID NO: 98 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 99 - protein, 15 aa, synthetic construct
ARGAPQGAAAGWFDP

SEQ ID NO: 100 - protein, 13 aa, synthetic construct
GAPQGAAAGWFDP

SEQ ID NO: 101 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 102 - protein, 15 aa, synthetic construct
ARGAPLGAAAGWFDP

SEQ ID NO: 103 - protein, 13 aa, synthetic construct
GAPLGAAAGWFDP

SEQ ID NO: 104 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 105 - protein, 15 aa, synthetic construct
ARGAPFGAAAGWFDP

SEQ ID NO: 106 - protein, 13 aa, synthetic construct
GAPFGAAAGWFDP

SEQ ID NO: 107 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 108 - protein, 15 aa, synthetic construct
ARGAPVGAAAGWFDP

SEQ ID NO: 109 - protein, 13 aa, synthetic construct
GAPVGAAAGWFDP

SEQ ID NO: 110 - protein, 122 aa, synthetic construct
  VARIANT 102: note = X is M, L, I, V, Q, or F
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 111 - protein, 15 aa, synthetic construct
  VARIANT 6: note = X is M, L, I, V, Q, or F
ARGAPXGAAAGWFDP

SEQ ID NO: 112 - protein, 13 aa, synthetic construct
  VARIANT 4: note = X is M, L, I, V, Q, or F
GAPXGAAAGWFDP

SEQ ID NO: 113 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 114 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 115 - protein, 5 aa, synthetic construct
SYAMS

SEQ ID NO: 116 - protein, 115 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 117 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 118 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 20 aa, synthetic construct
GGGGSGGGGSGGGGSGGGGS

SEQ ID NO: 120 - protein, 40 aa, synthetic construct
  source 1..40: note = up to 19 copies of GS could be deleted
GSGSGSGSGSGSGSGSGSGSGSGSGSGSGSGSGSGSGSGS

SEQ ID NO: 121 - protein, 60 aa, synthetic construct
  source 1..60: note = up to 19 copies of GGS could be deleted
GGSGGSGGSGGSGGSGGSGGSGGSGGSGGSGGSGGSGGSGGSGGSGGSGGSGGSGGSGGS

SEQ ID NO: 122 - protein, 4 aa, synthetic construct
  REPEAT 1..4: note = GGGS can be repeated n times, wherein n is 1, 2, 3, 4, 5, 6, 7, 8, 9, 10, 11, 12, 13, 14, 15, 16, 17, 18, 19, or 20
GGGS

SEQ ID NO: 123 - protein, 4 aa, synthetic construct
  REPEAT 1..4: note = GGSG can be repeated n times, wherein n is 1, 2, 3, 4, 5, 6, 7, 8, 9, 10, 11, 12, 13, 14, 15, 16, 17, 18, 19, or 20
GGSG

SEQ ID NO: 124 - protein, 5 aa, synthetic construct
  REPEAT 1..5: note = GGSGG can be repeated n times, wherein n is 1, 2, 3, 4, 5, 6, 7, 8, 9, 10, 11, 12, 13, 14, 15, 16, 17, 18, 19, or 20
GGSGG

SEQ ID NO: 125 - protein, 5 aa, synthetic construct
  REPEAT 1..5: note = GGGGS can be repeated n times, wherein n is 1, 2, 3, 4, 5, 6, 7, 8, 9, 10, 11, 12, 13, 14, 15, 16, 17, 18, 19, or 20
GGGGS

SEQ ID NO: 126 - protein, 20 aa, synthetic construct
GSGSGSGSGSGSGSGSGSGS

SEQ ID NO: 127 - protein, 30 aa, synthetic construct
GGSGGSGGSGGSGGSGGSGGSGGSGGSGGS

SEQ ID NO: 128 - protein, 40 aa, synthetic construct
GGGSGGGSGGGSGGGSGGGSGGGSGGGSGGGSGGGSGGGS

SEQ ID NO: 129 - protein, 40 aa, synthetic construct
GGSGGGSGGGSGGGSGGGSGGGSGGGSGGGSGGGSGGGSG

SEQ ID NO: 130 - protein, 50 aa, synthetic construct
GGSGGGGSGGGGSGGGGSGGGGSGGGGSGGGGSGGGGSGGGGSGGGGSGG

SEQ ID NO: 131 - protein, 50 aa, synthetic construct
GGGGSGGGGSGGGGSGGGGSGGGGSGGGGSGGGGSGGGGSGGGGSGGGGS

SEQ ID NO: 132 - protein, 15 aa, synthetic construct
GGGGSGGGGSGGGGS

SEQ ID NO: 133 - protein, 100 aa, synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 134 - protein, 100 aa, synthetic construct
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 135 - protein, 8 aa, synthetic construct
SSNSAAWG

SEQ ID NO: 136 - protein, 14 aa, synthetic construct
WLGRIYYRSKWYNS

SEQ ID NO: 137 - protein, 13 aa, synthetic construct
ARYQWVPKIGVFD

SEQ ID NO: 138 - protein, 252 aa, synthetic construct
QVQLQQSGPGLVKPSQTLSLTCAISGDSVSSNSAAWGWIRQSPGRCLEWLGRIYYRSKWYNSYAVSVKSRITINPDTSKNQFSLQLNSVTPEDTAVYYCARYQWVPKIGVFDSWGQGTLVTVSSGGGGSGGGGSGGGGSGGGGSDIVLTQSPATLSLSPGERATLSCRASQFILPEYLSWYQQKPGQAPRLLIYGSSSRATGVPARFSGSGSGTDFTLTISSLEPEDFAVYYCQQFYSSPLTFGCGTKVEIK

SEQ ID NO: 139 - protein, 252 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSQISPPTFGCGTKVEIKGGGGSGGGGSGGGGSGGGGSEVQLVESGGGLVQPGGSLRLSCAASGFTISSSSIHWVRQAPGKCLEWVAWVLPSVGFTDYADSVKGRFTISADTSKNTAYLQMNSLRAEDTAVYYCARRVCYNRLGVCAGGMDYWGQGTLVTVSS

SEQ ID NO: 140 - protein, 252 aa, synthetic construct
EVQLVESGGGLVQPGGSLRLSCAASGFTISSSSIHWVRQAPGKCLEWVAWVLPSVGFTDYADSVKGRFTISADTSKNTAYLQMNSLRAEDTAVYYCARRVCYNRLGVCAGGMDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQSQISPPTFGCGTKVEIK

SEQ ID NO: 141 - protein, 252 aa, synthetic construct
DIQMTQSPSSLSASVGDRVTITCRASEDISTAVAWYQQKPGKAPKLLIYAASFLYSGVPSRFSGSGSGTDFTLTISSLQPEDFATYYCQQSQISPPTFGCGTKVEIKGGGGSGGGGSGGGGSGGGGSEVQLVESGGGLVQPGGSLRLSCAASGFTISSSSIHWVRQAPGKCLEWVAWVLPSVGFTDYADSVKGRFTISADTSKNTAYLQMNSLRAEDTAVYYCARRVCYNRLGVCAGGMDYWGQGTLVTVSS

SEQ ID NO: 142 - protein, 252 aa, synthetic construct
EVQLVESGGGLVQPGGSLRLSCAASGFTISSSSIHWVRQAPGKCLEWVAWVLPSVGFTDYADSVKGRFTISADTSKNTAYLQMNSLRAEDTAVYYCARRVCYNRLGVCAGGMDYWGQGTLVTVSSGGGGSGGGGSGGGGSGGGGSDIQMTQSPSSLSASVGDRVTITCRASEDISTAVAWYQQKPGKAPKLLIYAASFLYSGVPSRFSGSGSGTDFTLTISSLQPEDFATYYCQQSQISPPTFGCGTKVEIK

SEQ ID NO: 143 - protein, 251 aa, synthetic construct
DIQMTQSPSSLSASVGDRVTITCKSSQSLLYSSNQNNYLAWYQQKPGKAPKLLIYWAQHLDSGVPSRFSGSGSGTDFTLTISSLQPEDFATYYCQQYYTYPYTFGCGTKVEIKGGGGSGGGGSGGGGSGGGGSEVQLVESGGGLVQPGGSLRLSCAASGLPMAGFYTSWVRQAPGKCLEWVGFIRDKANGYTTEYNPSVKGRFTISADTSKNTAYLQMNSLRAEDTAVYYCAQVRRALDYWGQGTLVTVSS

SEQ ID NO: 144 - protein, 251 aa, synthetic construct
EVQLVESGGGLVQPGGSLRLSCAASGLPMAGFYTSWVRQAPGKCLEWVGFIRDKANGYTTEYNPSVKGRFTISADTSKNTAYLQMNSLRAEDTAVYYCAQVRRALDYWGQGTLVTVSSGGGGSGGGGSGGGGSGGGGSDIQMTQSPSSLSASVGDRVTITCKSSQSLLYSSNQNNYLAWYQQKPGKAPKLLIYWAQHLDSGVPSRFSGSGSGTDFTLTISSLQPEDFATYYCQQYYTYPYTFGCGTKVEIK

SEQ ID NO: 145 - protein, 124 aa, synthetic construct
QVQLQQSGPGLVKPSQTLSLTCAISGDSVSSNSAAWGWIRQSPGRGLEWLGRIYYRSKWYNSYAVSVKSRITINPDTSKNQFSLQLNSVTPEDTAVYYCARYQWVPKIGVFDSWGQGTLVTVSS

SEQ ID NO: 146 - protein, 108 aa, synthetic construct
DIVLTQSPATLSLSPGERATLSCRASQFILPEYLSWYQQKPGQAPRLLIYGSSSRATGVPARFSGSGSGTDFTLTISSLEPEDFAVYYCQQFYSSPLTFGQGTKVEIK

SEQ ID NO: 147 - protein, 125 aa, synthetic construct
EVQLVESGGGLVQPGGSLRLSCAASGFTISSSSIHWVRQAPGKGLEWVAWVLPSVGFTDYADSVKGRFTISADTSKNTAYLQMNSLRAEDTAVYYCARRVCYNRLGVCAGGMDYWGQGTLVTVSS

SEQ ID NO: 148 - protein, 107 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSQISPPTFGQGTKVEIK

SEQ ID NO: 149 - protein, 249 aa, synthetic construct
EIVLTQSPATLSLSPGERATLSCRASESVDNYGISFLNWFQQKPGQAPRLLIYAASNRATGIPARFSGSGSGTDFTLTISSLEPEDFAVYYCQQSKEVPWTFGCGTKVEIKGGGGSGGGGSGGGGSGGGGSQVQLQESGPGLVKPSQTLSLTCTVSGDSITSGYWNWIRQHPGKCLEYIGYISYSGSTYYNPSLKSRVTISRDTSKNQFSLKLSSVTAADTAVYYCASPNYPFYAMDYWGQGTLVTVSS

SEQ ID NO: 150 - protein, 107 aa, synthetic construct
DIQMTQSPSSLSASVGDRVTITCRASEDISTAVAWYQQKPGKAPKLLIYAASFLYSGVPSRFSGSGSGTDFTLTISSLQPEDFATYYCQQSQISPPTFGQGTKVEIK

SEQ ID NO: 151 - protein, 118 aa, synthetic construct
EVQLVESGGGLVQPGGSLRLSCAASGLPMAGFYTSWVRQAPGKGLEWVGFIRDKANGYTTEYNPSVKGRFTISADTSKNTAYLQMNSLRAEDTAVYYCAQVRRALDYWGQGTLVTVSS

SEQ ID NO: 152 - protein, 113 aa, synthetic construct
DIQMTQSPSSLSASVGDRVTITCKSSQSLLYSSNQNNYLAWYQQKPGKAPKLLIYWAQHLDSGVPSRFSGSGSGTDFTLTISSLQPEDFATYYCQQYYTYPYTFGQGTKVEIK

SEQ ID NO: 153 - protein, 118 aa, Homo sapiens
QVQLQESGPGLVKPSQTLSLTCTVSGDSITSGYWNWIRQHPGKGLEYIGYISYSGSTYYNPSLKSRVTISRDTSKNQFSLKLSSVTAADTAVYYCASPNYPFYAMDYWGQGTLVTVSS

SEQ ID NO: 154 - protein, 111 aa, Homo sapiens
EIVLTQSPATLSLSPGERATLSCRASESVDNYGISFLNWFQQKPGQAPRLLIYAASNRATGIPARFSGSGSGTDFTLTISSLEPEDFAVYYCQQSKEVPWTFGGGTKVEIK

SEQ ID NO: 155 - protein, 118 aa, Homo sapiens
EVQLQESGPGLVKPSQTLSLTCTVSGDSITSGYWNWIRQHPGKGLEYIGYISYSGSTYYNPSLKSRVTISRDTSKNQYSLKLSSVTAADTAVYYCASPNYPFYAMDYWGQGTLVTVSS

SEQ ID NO: 156 - protein, 111 aa, Homo sapiens
DIVLTQSPATLSLSPGERATLSCRASESVDNYGISFMNWFQQKPGQAPRLLIYAASNRATGIPARFSGSGSGTDFTLTISSLEPEDFAVYYCQQSKEVPWTFGGGTKVEIK

SEQ ID NO: 157 - protein, 7 aa, synthetic construct
SNSAAWG

SEQ ID NO: 158 - protein, 18 aa, synthetic construct
RIYYRSKWYNSYAVSVKS

SEQ ID NO: 159 - protein, 12 aa, synthetic construct
YQWVPKIGVFDS

SEQ ID NO: 160 - protein, 12 aa, synthetic construct
RASQFILPEYLS

SEQ ID NO: 161 - protein, 7 aa, synthetic construct
GSSSRAT

SEQ ID NO: 162 - protein, 9 aa, synthetic construct
QQFYSSPLT

SEQ ID NO: 163 - protein, 6 aa, synthetic construct
TISSSS

SEQ ID NO: 164 - protein, 11 aa, synthetic construct
AWVLPSVGFTD

SEQ ID NO: 165 - protein, 13 aa, synthetic construct
RVCYNRLGVCAGG

SEQ ID NO: 166 - protein, 6 aa, synthetic construct
QDVSTA

SEQ ID NO: 167 - protein, 7 aa, synthetic construct
YSASFLY

SEQ ID NO: 168 - protein, 7 aa, synthetic construct
CQQSQIS

SEQ ID NO: 169 - protein, 6 aa, synthetic construct
EDISTA

SEQ ID NO: 170 - protein, 7 aa, synthetic construct
YAASFLY

SEQ ID NO: 171 - protein, 5 aa, synthetic construct
GFYTS

SEQ ID NO: 172 - protein, 19 aa, synthetic construct
FIRDKANGYTTEYNPSVKG

SEQ ID NO: 173 - protein, 7 aa, synthetic construct
VRRALDY

SEQ ID NO: 174 - protein, 17 aa, synthetic construct
KSSQSLLYSSNQNNYLA

SEQ ID NO: 175 - protein, 7 aa, synthetic construct
WAQHLDS

SEQ ID NO: 176 - protein, 9 aa, synthetic construct
QQYYTYPYT

SEQ ID NO: 177 - protein, 8 aa, Homo sapiens
GDSITSGY

SEQ ID NO: 178 - protein, 5 aa, Homo sapiens
SGYWN

SEQ ID NO: 179 - protein, 7 aa, Homo sapiens
ISYSGST

SEQ ID NO: 180 - protein, 16 aa, Homo sapiens
YISYSGSTYYNPSLKS

SEQ ID NO: 181 - protein, 12 aa, Homo sapiens
ASPNYPFYAMDY

SEQ ID NO: 182 - protein, 10 aa, Homo sapiens
PNYPFYAMDY

SEQ ID NO: 183 - protein, 10 aa, Homo sapiens
ESVDNYGISF

SEQ ID NO: 184 - protein, 15 aa, Homo sapiens
RASESVDNYGISFLN

SEQ ID NO: 185 - skipped

SEQ ID NO: 186 - protein, 7 aa, Homo sapiens
AASNRAT

SEQ ID NO: 187 - protein, 9 aa, Homo sapiens
QQSKEVPWT

SEQ ID NO: 188 - protein, 15 aa, Homo sapiens
RASESVDNYGISFMN

SEQ ID NO: 189 - protein, 184 aa, synthetic construct
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 190 - protein, 249 aa, synthetic construct
QVQLQESGPGLVKPSQTLSLTCTVSGDSITSGYWNWIRQHPGKCLEYIGYISYSGSTYYNPSLKSRVTISRDTSKNQFSLKLSSVTAADTAVYYCASPNYPFYAMDYWGQGTLVTVSSGGGGSGGGGSGGGGSGGGGSEIVLTQSPATLSLSPGERATLSCRASESVDNYGISFLNWFQQKPGQAPRLLIYAASNRATGIPARFSGSGSGTDFTLTISSLEPEDFAVYYCQQSKEVPWTFGCGTKVEIK

SEQ ID NO: 191 - protein, 249 aa, synthetic construct
DIVLTQSPATLSLSPGERATLSCRASESVDNYGISFMNWFQQKPGQAPRLLIYAASNRATGIPARFSGSGSGTDFTLTISSLEPEDFAVYYCQQSKEVPWTFGCGTKVEIKGGGGSGGGGSGGGGSGGGGSEVQLQESGPGLVKPSQTLSLTCTVSGDSITSGYWNWIRQHPGKCLEYIGYISYSGSTYYNPSLKSRVTISRDTSKNQYSLKLSSVTAADTAVYYCASPNYPFYAMDYWGQGTLVTVSS

SEQ ID NO: 192 - protein, 249 aa, synthetic construct
EVQLQESGPGLVKPSQTLSLTCTVSGDSITSGYWNWIRQHPGKCLEYIGYISYSGSTYYNPSLKSRVTISRDTSKNQYSLKLSSVTAADTAVYYCASPNYPFYAMDYWGQGTLVTVSSGGGGSGGGGSGGGGSGGGGSDIVLTQSPATLSLSPGERATLSCRASESVDNYGISFMNWFQQKPGQAPRLLIYAASNRATGIPARFSGSGSGTDFTLTISSLEPEDFAVYYCQQSKEVPWTFGCGTKVEIK

SEQ ID NO: 193 - protein, 480 aa, synthetic construct
DIVLTQSPATLSLSPGERATLSCRASQFILPEYLSWYQQKPGQAPRLLIYGSSSRATGVPARFSGSGSGTDFTLTISSLEPEDFAVYYCQQFYSSPLTFGCGTKVEIKGGGGSGGGGSGGGGSGGGGSQVQLQQSGPGLVKPSQTLSLTCAISGDSVSSNSAAWGWIRQSPGRCLEWLGRIYYRSKWYNSYAVSVKSRITINPDTSKNQFSLQLNSVTPEDTAVYYCARYQWVPKIGVFDSWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 194 - protein, 451 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 195 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 453 aa, synthetic construct
QVQLQQSGPGLVKPSQTLSLTCAISGDSVSSNSAAWGWIRQSPGRGLEWLGRIYYRSKWYNSYAVSVKSRITINPDTSKNQFSLQLNSVTPEDTAVYYCARYQWVPKIGVFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 197 - protein, 215 aa, synthetic construct
DIVLTQSPATLSLSPGERATLSCRASQFILPEYLSWYQQKPGQAPRLLIYGSSSRATGVPARFSGSGSGTDFTLTISSLEPEDFAVYYCQQFYSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 477 aa, synthetic construct
EIVLTQSPATLSLSPGERATLSCRASESVDNYGISFLNWFQQKPGQAPRLLIYAASNRATGIPARFSGSGSGTDFTLTISSLEPEDFAVYYCQQSKEVPWTFGCGTKVEIKGGGGSGGGGSGGGGSGGGGSQVQLQESGPGLVKPSQTLSLTCTVSGDSITSGYWNWIRQHPGKCLEYIGYISYSGSTYYNPSLKSRVTISRDTSKNQFSLKLSSVTAADTAVYYCASPNYPFYAMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 199 - protein, 447 aa, synthetic construct
EVQLQESGPGLVKPSQTLSLTCTVSGDSITSGYWNWIRQHPGKGLEYIGYISYSGSTYYNPSLKSRVTISRDTSKNQYSLKLSSVTAADTAVYYCASPNYPFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 200 - protein, 218 aa, synthetic construct
DIVLTQSPATLSLSPGERATLSCRASESVDNYGISFMNWFQQKPGQAPRLLIYAASNRATGIPARFSGSGSGTDFTLTISSLEPEDFAVYYCQQSKEVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 480 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSQISPPTFGCGTKVEIKGGGGSGGGGSGGGGSGGGGSEVQLVESGGGLVQPGGSLRLSCAASGFTISSSSIHWVRQAPGKCLEWVAWVLPSVGFTDYADSVKGRFTISADTSKNTAYLQMNSLRAEDTAVYYCARRVCYNRLGVCAGGMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 202 - protein, 454 aa, synthetic construct
EVQLVESGGGLVQPGGSLRLSCAASGFTISSSSIHWVRQAPGKGLEWVAWVLPSVGFTDYADSVKGRFTISADTSKNTAYLQMNSLRAEDTAVYYCARRVCYNRLGVCAGGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 203 - protein, 214 aa, synthetic construct [sequence omitted]

SEQ ID NO: 204 - protein, 480 aa, synthetic construct
DIQMTQSPSSLSASVGDRVTITCRASEDISTAVAWYQQKPGKAPKLLIYAASFLYSGVPSRFSGSGSGTDFTLTISSLQPEDFATYYCQQSQISPPTFGCGTKVEIKGGGGSGGGGSGGGGSGGGGSEVQLVESGGGLVQPGGSLRLSCAASGFTISSSSIHWVRQAPGKCLEWVAWVLPSVGFTDYADSVKGRFTISADTSKNTAYLQMNSLRAEDTAVYYCARRVCYNRLGVCAGGMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 205 - protein, 220 aa, synthetic construct
DIQMTQSPSSLSASVGDRVTITCKSSQSLLYSSNQNNYLAWYQQKPGKAPKLLIYWAQHLDSGVPSRFSGSGSGTDFTLTISSLQPEDFATYYCQQYYTYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 214 aa, synthetic construct
DIQMTQSPSSLSASVGDRVTITCRASEDISTAVAWYQQKPGKAPKLLIYAASFLYSGVPSRFSGSGSGTDFTLTISSLQPEDFATYYCQQSQISPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 252 aa, synthetic construct
DIVLTQSPATLSLSPGERATLSCRASQFILPEYLSWYQQKPGQAPRLLIYGSSSRATGVPARFSGSGSGTDFTLTISSLEPEDFAVYYCQQFYSSPLTFGCGTKVEIKGGGGSGGGGSGGGGSGGGGSQVQLQQSGPGLVKPSQTLSLTCAISGDSVSSNSAAWGWIRQSPGRCLEWLGRIYYRSKWYNSYAVSVKSRITINPDTSKNQFSLQLNSVTPEDTAVYYCARYQWVPKIGVFDSWGQGTLVTVSS

SEQ ID NO: 208 - protein, 447 aa, synthetic construct
QVQLQESGPGLVKPSQTLSLTCTVSGDSITSGYWNWIRQHPGKGLEYIGYISYSGSTYYNPSLKSRVTISRDTSKNQFSLKLSSVTAADTAVYYCASPNYPFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 209 - protein, 218 aa, synthetic construct
EIVLTQSPATLSLSPGERATLSCRASESVDNYGISFLNWFQQKPGQAPRLLIYAASNRATGIPARFSGSGSGTDFTLTISSLEPEDFAVYYCQQSKEVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 477 aa, synthetic construct
DIVLTQSPATLSLSPGERATLSCRASESVDNYGISFMNWFQQKPGQAPRLLIYAASNRATGIPARFSGSGSGTDFTLTISSLEPEDFAVYYCQQSKEVPWTFGCGTKVEIKGGGGSGGGGSGGGGSGGGGSEVQLQESGPGLVKPSQTLSLTCTVSGDSITSGYWNWIRQHPGKCLEYIGYISYSGSTYYNPSLKSRVTISRDTSKNQYSLKLSSVTAADTAVYYCASPNYPFYAMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 211 - protein, 479 aa, synthetic construct
DIQMTQSPSSLSASVGDRVTITCKSSQSLLYSSNQNNYLAWYQQKPGKAPKLLIYWAQHLDSGVPSRFSGSGSGTDFTLTISSLQPEDFATYYCQQYYTYPYTFGCGTKVEIKGGGGSGGGGSGGGGSGGGGSEVQLVESGGGLVQPGGSLRLSCAASGLPMAGFYTSWVRQAPGKCLEWVGFIRDKANGYTTEYNPSVKGRFTISADTSKNTAYLQMNSLRAEDTAVYYCAQVRRALDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 212 - protein, 447 aa, synthetic construct
EVQLVESGGGLVQPGGSLRLSCAASGLPMAGFYTSWVRQAPGKGLEWVGFIRDKANGYTTEYNPSVKGRFTISADTSKNTAYLQMNSLRAEDTAVYYCAQVRRALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 213 - protein, 451 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 214 - protein, 7 aa, synthetic construct
GFTFSSY

SEQ ID NO: 215 - protein, 6 aa, synthetic construct
WYDGSN

SEQ ID NO: 216 - protein, 17 aa, synthetic construct
RFTMLRGLIIEDYGMDV

SEQ ID NO: 217 - protein, 11 aa, synthetic construct
RASQSISSYLN

SEQ ID NO: 218 - protein, 9 aa, synthetic construct
QQSYSTPLT

SEQ ID NO: 219 - protein, 17 aa, synthetic construct
RFTMLRGVFIEDYGMDV

SEQ ID NO: 220 - protein, 7 aa, synthetic construct
GFTFSTY

SEQ ID NO: 221 - protein, 17 aa, synthetic construct
RNTMVRGVIIEDYGMDV

SEQ ID NO: 222 - protein, 9 aa, synthetic construct
QQSYSSPLT

SEQ ID NO: 223 - protein, 17 aa, synthetic construct
RFTMLRGQYIEDYGMDV

SEQ ID NO: 224 - protein, 17 aa, synthetic construct
RFTMLRGWYIEDYGMDV

SEQ ID NO: 225 - protein, 17 aa, synthetic construct
RFTMLRGWIIEDYGMDV

SEQ ID NO: 226 - protein, 17 aa, synthetic construct
  VARIANT 8: note = X is Q or W
  VARIANT 9: note = X is I or Y
RFTMLRGXXIEDYGMDV

SEQ ID NO: 227 - protein, 7 aa, synthetic construct
GFPFSSY

SEQ ID NO: 228 - protein, 7 aa, synthetic construct
GEWFSSY

SEQ ID NO: 229 - protein, 17 aa, synthetic construct
RFTHLRGWIIEDYGMDV

SEQ ID NO: 230 - protein, 7 aa, synthetic construct
GFTFSSS

SEQ ID NO: 231 - protein, 7 aa, synthetic construct
  VARIANT 2: note = X is F or E
  VARIANT 3: note = X is T, P, or W
  VARIANT 7: note = X is Y or S
GXXFSSX

SEQ ID NO: 232 - protein, 17 aa, synthetic construct
  VARIANT 4: note = X is M or H
  VARIANT 9: note = X is I or Y
RFTXLRGWXIEDYGMDV

SEQ ID NO: 233 - protein, 6 aa, synthetic construct
WYDASN

SEQ ID NO: 234 - protein, 17 aa, synthetic construct
RFTYLRGWYIEDYGLDV

SEQ ID NO: 235 - protein, 17 aa, synthetic construct
RFTSLRGWYIEDYGLDV

SEQ ID NO: 236 - protein, 17 aa, synthetic construct
  VARIANT 4: note = X is R, Y, or S
RFTXLRGWYIEDYGLDV

SEQ ID NO: 237 - protein, 17 aa, synthetic construct
RFTHLRGWYIEDYGLDV

SEQ ID NO: 238 - protein, 7 aa, synthetic construct
GFAFSSY

SEQ ID NO: 239 - protein, 6 aa, synthetic construct
WYDESN

SEQ ID NO: 240 - protein, 17 aa, synthetic construct
RFTNLRGWIIEDYGLDV

SEQ ID NO: 241 - protein, 7 aa, synthetic construct
GFTFSMY

SEQ ID NO: 242 - protein, 17 aa, synthetic construct
RFTRLRGWYIEDYGLDV

SEQ ID NO: 243 - protein, 7 aa, synthetic construct
GFTFGSY

SEQ ID NO: 244 - protein, 17 aa, synthetic construct
RFTHLRGQYIEDYGMDV

SEQ ID NO: 245 - protein, 7 aa, synthetic construct
  VARIANT 3: note = X is T or A
  VARIANT 5: note = X is S or G
  VARIANT 6: note = X is S or M
GFXFXXY

SEQ ID NO: 246 - protein, 6 aa, synthetic construct
  VARIANT 4: note = X is G, A, or E
WYDXSN

SEQ ID NO: 247 - protein, 17 aa, synthetic construct
  VARIANT 4: note = X is H, N, or R
  VARIANT 8: note = X is W or Q
  VARIANT 9: note = X is I or Y
  VARIANT 15: note = X is M or L
RFTXLRGXXIEDYGXDV

SEQ ID NO: 248 - protein, 17 aa, synthetic construct
RFTHLRGQYIEDYGLDV

SEQ ID NO: 249 - protein, 9 aa, synthetic construct
QQSYSIPLT

SEQ ID NO: 250 - protein, 126 aa, synthetic construct
EVQLVQSGGGVVQPGRSLRLSCAASGFTFSSYGMHWVRQAPGKGLEWVAVIWYDASNKYYGDSVKGRFTISRDNSKNTLYLQMNSLRDEDTAVYYCARRFTHLRGQYIEDYGLDVWGQGTTVTVSS

SEQ ID NO: 251 - protein, 107 aa, synthetic construct
EIVLTQSPSSLSASVGDRVTITCRASQSISSYLNWYQQKPGKAPKLLIYAASSLQSGVPSRFSGSGSGTDFTLTISSLQPEDFATYYCQQSYSIPLTFGGGTKVEIK

SEQ ID NO: 252 - protein, 126 aa, synthetic construct
EVQLVQSGGGVVQPGRSLRLSCAASGFTFSSYGMHWVRQAPGKCLEWVAVIWYDASNKYYGDSVKGRFTISRDNSKNTLYLQMNSLRDEDTAVYYCARRFTHLRGQYIEDYGLDVWGQGTTVTVSS

SEQ ID NO: 253 - protein, 107 aa, synthetic construct
EIVLTQSPSSLSASVGDRVTITCRASQSISSYLNWYQQKPGKAPKLLIYAASSLQSGVPSRFSGSGSGTDFTLTISSLQPEDFATYYCQQSYSIPLTFGCGTKVEIK

SEQ ID NO: 254 - protein, 253 aa, synthetic construct
EVQLVQSGGGVVQPGRSLRLSCAASGFTFSSYGMHWVRQAPGKCLEWVAVIWYDASNKYYGDSVKGRFTISRDNSKNTLYLQMNSLRDEDTAVYYCARRFTHLRGQYIEDYGLDVWGQGTTVTVSSGGGGSGGGGSGGGGSGGGGSEIVLTQSPSSLSASVGDRVTITCRASQSISSYLNWYQQKPGKAPKLLIYAASSLQSGVPSRFSGSGSGTDFTLTISSLQPEDFATYYCQQSYSIPLTFGCGTKVEIK

SEQ ID NO: 255 - protein, 253 aa, synthetic construct
EIVLTQSPSSLSASVGDRVTITCRASQSISSYLNWYQQKPGKAPKLLIYAASSLQSGVPSRFSGSGSGTDFTLTISSLQPEDFATYYCQQSYSIPLTFGCGTKVEIKGGGGSGGGGSGGGGSGGGGSEVQLVQSGGGVVQPGRSLRLSCAASGFTFSSYGMHWVRQAPGKCLEWVAVIWYDASNKYYGDSVKGRFTISRDNSKNTLYLQMNSLRDEDTAVYYCARRFTHLRGQYIEDYGLDVWGQGTTVTVSS

SEQ ID NO: 256 - protein, 7 aa, synthetic construct
  VARIANT 5: note = X is S or G
GFTFXSY

SEQ ID NO: 257 - protein, 6 aa, synthetic construct
  VARIANT 4: note = X is G or A
WYDXSN

SEQ ID NO: 258 - protein, 17 aa, synthetic construct
  VARIANT 4: note = X is M or H
  VARIANT 8: note = X is L, W, or Q
  VARIANT 9: note = X is I or Y
  VARIANT 15: note = X is M or L
RFTXLRGXXIEDYGXDV

SEQ ID NO: 259 - protein, 9 aa, synthetic construct
  VARIANT 6: note = X is T or I
QQSYSXPLT

SEQ ID NO: 260 - skipped

SEQ ID NO: 261 - protein, 17 aa, synthetic construct
  VARIANT 4: note = X is M, H, N, R, Y, or S
  VARIANT 8: note = X is L, Q, or W
  VARIANT 9: note = X is I or Y
  VARIANT 15: note = X is M or L
RFTXLRGXXIEDYGXDV

SEQ ID NO: 262 - protein, 112 aa, synthetic construct
DIVMTQTPLSLPVSLGDQASISCRSSQSLLHSNGNTYLYWYLQKPGQSPKLLIHRVSNRFSGVPDRFGGSGSGTDFTLKIIRVEAEDLGVYFCFQGTHVPWTFGGGTKLEIK

SEQ ID NO: 263 - protein, 121 aa, synthetic construct
QVQLQQPGAELVKPGASVKLSCKASGYTFTSYWMHWVKQRPGQGLEWIGEIDPFDSYTNYNQNFKGKATLTVDKSSSTAYMLLSSLTSDDSAVYYCARERLRLWSYYFDYWGQGTTLTVSS

SEQ ID NO: 264 - protein, 112 aa, synthetic construct
DVVMTQTPLSLPVSLGDQASISCRSSQSIVHSNGNTYLEWYLQKPGQSPKLLIYKVSNRLSGVPDRFSGSGSGTDFTLKISRVEAEDLGVYYCFQGSHDPFTFGSGTKLEIK

SEQ ID NO: 265 - protein, 121 aa, synthetic construct
QVQLQQPGAELVKPGASVKLSCKASGYTFTSYWMHWVKQRPGQGLEWIGEVDPSDSYTNYNQKFKGKATLTVDKSSSTAYILLSNLTSDDSAVYYCARERVRLWSYFFDYWGQGTTLTVSS

SEQ ID NO: 266 - protein, 112 aa, synthetic construct; identical to SEQ ID NO: 264

SEQ ID NO: 267 - protein, 124 aa, synthetic construct
EVQLVESGGGLVKPGGSLKLSCVVSGFTFSNYAMSWVRQTPEKRLEWVATISDGGGYTYYPDSVKGRFTISRDNAKNNLYLQMSHLKSEDTAIYYCARDDLGGGNYVSSYFDVWGTGTTVTVSS

SEQ ID NO: 268 - protein, 112 aa, synthetic construct
DVVMTQTPLSLPVSPGDQASISCRSSQSLVHSNGNTYLYWYLQKPGQSPKLLIYRVSNRFSGVPDRFSGSGSGTDFTLKINRVEAEDLGVYFCFQGTHVPLTFGSGTKLELK

SEQ ID NO: 269 - protein, 125 aa, synthetic construct
EVQLVESGGGLVKPGGSLKLSCAASGFSFSRYAMSWVRQTPEKRLEWVATISDGGSYTHYRDNVKGRFTISRDNAKNNLNLQMSHLKSEDTAIYYCARNEMGLYFDYDVYAMDYWGQGTSVTVSS

SEQ ID NO: 270 - protein, 481 aa, synthetic construct
EVQLVQSGGGVVQPGRSLRLSCAASGFTFSSYGMHWVRQAPGKCLEWVAVIWYDASNKYYGDSVKGRFTISRDNSKNTLYLQMNSLRDEDTAVYYCARRFTHLRGQYIEDYGLDVWGQGTTVTVSSGGGGSGGGGSGGGGSGGGGSEIVLTQSPSSLSASVGDRVTITCRASQSISSYLNWYQQKPGKAPKLLIYAASSLQSGVPSRFSGSGSGTDFTLTISSLQPEDFATYYCQQSYSIPLTFGCGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 271 - protein, 712 aa, synthetic construct
EVQLVQSGGGVVQPGRSLRLSCAASGFTFSSYGMHWVRQAPGKGLEWVAVIWYDASNKYYGDSVKGRFTISRDNSKNTLYLQMNSLRDEDTAVYYCARRFTHLRGQYIEDYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPGSGSGGGGSDIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPIGAAAGWFDPWGQGTLVTVSS

SEQ ID NO: 272 - protein, 455 aa, synthetic construct
EVQLVQSGGGVVQPGRSLRLSCAASGFTFSSYGMHWVRQAPGKGLEWVAVIWYDASNKYYGDSVKGRFTISRDNSKNTLYLQMNSLRDEDTAVYYCARRFTHLRGQYIEDYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 273 - protein, 214 aa, synthetic construct
EIVLTQSPSSLSASVGDRVTITCRASQSISSYLNWYQQKPGKAPKLLIYAASSLQSGVPSRFSGSGSGTDFTLTISSLQPEDFATYYCQQSYSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 274 - protein, 8 aa, synthetic construct
SGSGGGGS

SEQ ID NO: 275 - protein, 249 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPIGAAAGWFDPWGQGTLVTVSS

SEQ ID NO: 276 - protein, 107 aa, synthetic construct
EIVLTQSPSSLSASVGDRVTITCRASQSISSYLNWYQQKPGKAPKLLIYAASSLQSGVPSRFSGSGSGTDFTLTISSLQPEDFATYYCQQSYSTPLTFGCGTKVEIK

SEQ ID NO: 277 - protein, 126 aa, synthetic construct
EVQLVQSGGGVVQPGRSLRLSCAASGFTFSSYGMHWVRQAPGKCLEWVAVIWYDGSNKYYGDSVKGRFTISRDNSKNTLYLQMNSLRDEDTAVYYCARRFTMLRGQYIEDYGMDVWGQGTTVTVSS

SEQ ID NO: 278 - protein, 126 aa, synthetic construct
EVQLVQSGGGVVQPGRSLRLSCAASGFTFSSYGMHWVRQAPGKCLEWVAVIWYDGSNKYYGDSVKGRFTISRDNSKNTLYLQMNSLRDEDTAVYYCARRFTMLRGWYIEDYGMDVWGQGTTVTVSS

SEQ ID NO: 279 - protein, 126 aa, synthetic construct
EVQLVQSGGGVVQPGRSLRLSCAASGFTFSSYGMHWVRQAPGKCLEWVAVIWYDGSNKYYGDSVKGRFTISRDNSKNTLYLQMNSLRDEDTAVYYCARRFTMLRGWIIEDYGMDVWGQGTTVTVSS

SEQ ID NO: 280 - protein, 126 aa, synthetic construct
EVQLVQSGGGVVQPGRSLRLSCAASGFPFSSYGMHWVRQAPGKCLEWVAVIWYDGSNKYYGDSVKGRFTISRDNSKNTLYLQMNSLRDEDTAVYYCARRFTMLRGWYIEDYGMDVWGQGTTVTVSS

SEQ ID NO: 281 - protein, 126 aa, synthetic construct
EVQLVQSGGGVVQPGRSLRLSCAASGEWFSSYGMHWVRQAPGKCLEWVAVIWYDGSNKYYGDSVKGRFTISRDNSKNTLYLQMNSLRDEDTAVYYCARRFTHLRGWIIEDYGMDVWGQGTTVTVSS

SEQ ID NO: 282 - protein, 126 aa, synthetic construct
EVQLVQSGGGVVQPGRSLRLSCAASGFTFSSSGMHWVRQAPGKCLEWVAVIWYDGSNKYYGDSVKGRFTISRDNSKNTLYLQMNSLRDEDTAVYYCARRFTMLRGWYIEDYGMDVWGQGTTVTVSS

SEQ ID NO: 283 - protein, 126 aa, synthetic construct
EVQLVQSGGGVVQPGRSLRLSCAASGFTFSSSGMHWVRQAPGKCLEWVAVIWYDASNKYYGDSVKGRFTISRDNSKNTLYLQMNSLRDEDTAVYYCARRFTRLRGWYIEDYGLDVWGQGTTVTVSS

SEQ ID NO: 284 - protein, 126 aa, synthetic construct
EVQLVQSGGGVVQPGRSLRLSCAASGFTFSSSGMHWVRQAPGKCLEWVAVIWYDASNKYYGDSVKGRFTISRDNSKNTLYLQMNSLRDEDTAVYYCARRFTYLRGWYIEDYGLDVWGQGTTVTVSS

SEQ ID NO: 285 - protein, 126 aa, synthetic construct
EVQLVQSGGGVVQPGRSLRLSCAASGFTFSSSGMHWVRQAPGKCLEWVAVIWYDASNKYYGDSVKGRFTISRDNSKNTLYLQMNSLRDEDTAVYYCARRFTSLRGWYIEDYGLDVWGQGTTVTVSS

SEQ ID NO: 286 - protein, 126 aa, synthetic construct
EVQLVQSGGGVVQPGRSLRLSCAASGFTFSSYGMHWVRQAPGKCLEWVAVIWYDASNKYYGDSVKGRFTISRDNSKNTLYLQMNSLRDEDTAVYYCARRFTHLRGWYIEDYGLDVWGQGTTVTVSS

SEQ ID NO: 287 - protein, 126 aa, synthetic construct
EVQLVQSGGGVVQPGRSLRLSCAASGFAFSSYGMHWVRQAPGKCLEWVAVIWYDESNKYYGDSVKGRFTISRDNSRNTLYLQMNSLRDEDTAVYYCARRFTNLRGWIIEDYGLDVWGQGTTVTVSS

SEQ ID NO: 288 - protein, 126 aa, synthetic construct
EVQLVQSGGGVVQPGRSLRLSCAASGFTFSMYGMHWVRQAPGKCLEWVAVIWYDASNKYYGDSVKGRFTISRDNSKNTLYLQMNSLRDEDTAVYYCARRFTRLRGWYIEDYGLDVWGQGTTVTVSS

SEQ ID NO: 289 - protein, 126 aa, synthetic construct
EVQLVQSGGGVVQPGRSLRLSCAASGFTFGSYGMHWVRQAPGKCLEWVAVIWYDGSNKYYGDSVKGRFTISRDNSKNTLYLQMNSLRDEDTAVYYCARRFTHLRGQYIEDYGMDVWGQGTTVTVSS

SEQ ID NO: 290 - protein, 6 aa, synthetic construct
SSSSSY

SEQ ID NO: 291 - protein, 7 aa, synthetic construct
GYTFTSY

SEQ ID NO: 292 - protein, 6 aa, synthetic construct
DPFDSY

SEQ ID NO: 293 - protein, 12 aa, synthetic construct
ERLRLWSYYFDY

SEQ ID NO: 294 - protein, 16 aa, synthetic construct
RSSQSIVHSNGNTYLE

SEQ ID NO: 295 - protein, 7 aa, synthetic construct
KVSNRLS

SEQ ID NO: 296 - protein, 9 aa, synthetic construct
FQGSHDPFT

SEQ ID NO: 297 - protein, 6 aa, synthetic construct
DPSDSY

SEQ ID NO: 298 - protein, 12 aa, synthetic construct
ERVRLWSYFFDY

SEQ ID NO: 299 - protein, 7 aa, synthetic construct
GFTFSNY

SEQ ID NO: 300 - protein, 5 aa, synthetic construct
DGGGY

SEQ ID NO: 301 - protein, 15 aa, synthetic construct
DDLGGGNYVSSYFDV

SEQ ID NO: 302 - protein, 16 aa, synthetic construct
RSSQSLVHSNGNTYLY

SEQ ID NO: 303 - protein, 7 aa, synthetic construct
RVSNRFS

SEQ ID NO: 304 - protein, 9 aa, synthetic construct
FQGTHVPLT

SEQ ID NO: 305 - protein, 7 aa, synthetic construct
GFSFSRY

SEQ ID NO: 306 - protein, 5 aa, synthetic construct
DGGSY

SEQ ID NO: 307 - protein, 16 aa, synthetic construct
NEMGLYFDYDVYAMDY

SEQ ID NO: 308 - protein, 16 aa, synthetic construct
RSSQSLLHSNGNTYLY

SEQ ID NO: 309 - protein, 9 aa, synthetic construct
FQGTHVPWT

SEQ ID NO: 310 - protein, 126 aa, synthetic construct
EVQLVQSGGGVVQPGRSLRLSCAASGFTFSSYGMHWVRQAPGKCLEWVAVIWYDGSNKYYGDSVKGRFTISRDNSKNTLYLQMNSLRDEDTAVYYCARRFTMLRGLIIEDYGMDVWGQGTTVTVSS

(19) United States
(12) Patent Application Publication
(10) Pub. No.: US 2024/0270861 A1
(43) Pub. Date: Aug. 15, 2024

(54) PROTEINS BINDING NKG2D, CD16 AND BAFF-R

(71) Applicant: Dragonfly Therapeutics, Inc., Waltham, MA (US)

(72) Inventors: Aaron BELLI, Acton, MA (US); Ann F. CHEUNG, Lincoln, MA (US); Stacey V. DRABIC, Cambridge, MA (US); Daniel FALLON, Winchester, MA (US); Benjamin FISCHER, Waltham, MA (US); Asya GRINBERG, Lexington, MA (US); Pyae P. HEIN, Malden, MA (US); Alexander IVANOV, Waltham, MA (US); Zong Sean JUO, Taipei (TW); Mark LEWANDOWSKI, Auburndale, MA (US); Xinbi LI, Waltham, MA (US); Matthew SCHNEIDER, Natick, MA (US)

(21) Appl. No.: 18/620,824

(22) Filed: Mar. 28, 2024

Related U.S. Application Data
(63) Continuation of application No. PCT/US2022/077083, filed on Sep. 27, 2022.
(60) Provisional application No. 63/250,160, filed on Sep. 29, 2021.

(51) Int. Cl. C07K 16/28; A61P 35/00

(52) U.S. Cl. CPC C07K 16/2878; A61P 35/00; C07K 16/283; C07K 16/2851; C07K 2317/31; C07K 2317/52; C07K 2317/55; C07K 2317/565; C07K 2317/622; C07K 2317/732; C07K 2317/76; C07K 2319/21

(57) ABSTRACT

Multispecific binding proteins that bind NKG2D receptor, CD16, and B cell-activating factor receptor (BAFF-R) are described, as well as pharmaceutical compositions and therapeutic methods of the multispecific binding proteins useful for the treatment of cancer and autoimmune inflammatory diseases.

### CROSS REFERENCE

This application is a continuation of International Patent Application No. PCT/US2022/077083, filed Sep. 27, 2022, which claims the benefit of U.S. Provisional Application No. 63/250,160, filed Sep. 29, 2021. The entire contents of each of the foregoing applications are incorporated herein by reference in their entirety.

### SEQUENCE LISTING

This application contains a computer readable Sequence Listing which has been submitted in XML file format via Patent Center, the entire content of which is incorporated by reference herein in its entirety. The Sequence Listing XML file submitted via Patent Center is entitled “14247-700-999_SequenceListing.xml,” was created on Mar. 27, 2024, and is 305,046 bytes in size.

### FIELD OF THE INVENTION

The present application relates to multispecific binding proteins that bind to NKG2D, CD16, and B cell-activating factor receptor (BAFF-R) on a cell, pharmaceutical compositions comprising such proteins, and therapeutic methods using such proteins and pharmaceutical compositions, including for the treatment of cancer.

### BACKGROUND

Despite substantial research efforts, cancer continues to be a significant clinical and financial burden in countries across the globe. According to the World Health Organization (WHO), it is the second leading cause of death. Surgery, radiation therapy, chemotherapy, biological therapy, immunotherapy, hormone therapy, stem-cell transplantation, and precision medicine are among the existing treatment modalities. Despite extensive research in these areas, a highly effective, curative solution, particularly for the most aggressive cancers, has yet to be identified. Furthermore, many of the existing anti-cancer treatment modalities have substantial adverse side effects.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perforin and granzymes as well as via death receptor pathways. Activated NK cells also secrete inflammatory cytokines such as IFN-7 and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD16 receptors on their surface. The overall sensitivity of NK cells to activation depends on the sum of stimulatory and inhibitory signals. NKG2D is a type-II transmembrane protein that is expressed by essentially all natural killer cells where NKG2D serves as an activating receptor. NKG2D is also be found on T cells where it acts as a costimulatory receptor. The ability to modulate NK cell function via NKG2D is useful in various therapeutic contexts including malignancy.

BAFF-R, also called BAFF receptor, TNF receptor superfamily member 13C (TNFRSF13C), CD268, or BR3, is a type III transmembrane protein of the TNF receptor superfamily. BAFF-R is expressed at the late transitional (T2) B-cell stage and on all mature B cells, is downregulated on germinal center B cells, is re-expressed on memory cells, and is absent on plasma cells (Davidson (2012) Curr. Rheumatol. Rep., 14(4): 295-302). BAFF-R is a receptor for B cell-activating factor (BAFF), a B cell survival factor. BAFF can engage three receptors: BAFF-R, transmembrane activator and CAML interactor (TACI), and B-cell maturation antigen (BCMA). Among these three receptors, BAFF-R is the principal receptor involved in the development of follicular and marginal zone splenic B cells (Schiemann et al. (2001) Science, 293: 2111-14).

The BAFF/BAFF-R signaling axis may play a role in B cell hyperplasia. Increased expression of BAFF-R, as well as elevated serum levels of BAFF, has been observed in non-Hodgkin lymphoma (NHL) patients (Shen et al. (2016) Adv. Clin. Exp. Med., 25(5):837-44). Certain single nucleotide polymorphisms (SNPs) in BAFF-R are associated with increased risk of chronic lymphocytic leukemia (CLL) (Jesek et al. (2016) Tumour Biol., 37(10):13617-26). The BAFF/BAFF-R axis is also implicated in autoimmune inflammatory diseases (Mackay et al. (1999) J. Exp. Med., 190:1697-1710). Some systemic lupus erythematosus (SLE) patients have increased levels of BAFF in serum (Cheema et al. (2001) Arthritis Rheum., 44:1313-19), and BAFF-R is consistently occupied on blood B cells in SLE (Carter et al. (2005) Arthritis Rheum., 52:3943-54). Given the observation that autoreactive B cells have a greater dependency on BAFF for their survival as compared with protective B cells (Lesley et al. (2004) Immunity, 20:441-53), it has been proposed that abnormally high levels of BAFF may contribute to the pathogenesis of autoimmune diseases by enhancing the survival of autoreactive B cells.

Therefore, there remains a need in the field for new and useful proteins that bind BAFF-R for use in treatment of cancer and autoimmune inflammatory diseases.

### SUMMARY

The present application provides multispecific binding proteins that bind to the NKG2D receptor and CD16 receptor on natural killer cells, and BAFF-R. Such proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys. Formulations containing any one of the proteins disclosed herein; cells containing one or more nucleic acids expressing the proteins, and methods of enhancing tumor cell death using the proteins are also provided.

Accordingly, in one aspect, the present application provides a protein comprising:
(a) a first antigen-binding site that binds NKG2D;
(b) a second antigen-binding site that binds B cell-activating factor receptor (BAFF-R); and
(c) an antibody Fc domain or a portion thereof sufficient to bind CD16, or a third antigen-binding site that binds CD16.

In some embodiments of a protein disclosed herein, the first antigen-binding site that binds NKG2D is a Fab fragment, and the second antigen-binding site that binds BAFF-R is an scFv. In some embodiments, the first antigen-binding site that binds NKG2D is an scFv, and the second antigen-binding site that binds BAFF-R is a Fab fragment.

In some embodiments of a protein disclosed herein, the protein further comprises an additional antigen-binding site that binds BAFF-R. In certain embodiments, the first antigen-binding site that binds NKG2D is an scFv, and the second and the additional antigen-binding sites that bind BAFF-R are each a Fab fragment. In certain embodiments, the first antigen-binding site that binds NKG2D is an scFv, and the second and the additional antigen-binding sites that bind BAFF-R are each an scFv. In certain embodiments, the amino acid sequences of the second and the additional antigen-binding sites are identical. In certain embodiments, the amino acid sequences of the second and the additional antigen-binding sites are different.

In some embodiments of a protein disclosed herein, the scFv that binds NKG2D is linked to an antibody constant domain or a portion thereof sufficient to bind CD16, via a hinge comprising Ala-Ser or Gly-Ser, wherein the scFv comprises a heavy chain variable domain and a light chain variable domain. In certain embodiments, each scFv that binds BAFF-R is linked to an antibody constant domain or a portion thereof sufficient to bind CD16, via a hinge comprising Ala-Ser or Gly-Ser, wherein the scFv comprises a heavy chain variable domain and a light chain variable domain. In certain embodiments, the hinge further comprises an amino acid sequence Thr-Lys-Gly.

In some embodiments of a protein disclosed herein, within the scFv that binds NKG2D, the heavy chain variable domain of the scFv forms a disulfide bridge with the light chain variable domain of the scFv. In some embodiments, within each scFv that binds BAFF-R, the heavy chain variable domain of the scFv forms a disulfide bridge with the light chain variable domain of the scFv. In some embodiments, the disulfide bridge is formed between C44 of the heavy chain variable domain and C100 of the light chain variable domain, numbered under the Kabat numbering scheme. In some embodiments, within the scFv that binds NKG2D, the heavy chain variable domain is linked to the light chain variable domain via a flexible linker. In some embodiments, within each scFv that binds BAFF-R, the heavy chain variable domain is linked to the light chain variable domain via a flexible linker. In certain embodiments, the flexible linker comprises (G₄S)₄. In certain embodiments, within the scFv that binds NKG2D, the heavy chain variable domain is positioned at the C-terminus of the light chain variable domain. In certain embodiments, within each scFv that binds BAFF-R, the heavy chain variable domain is positioned at the C-terminus of the light chain variable domain. In certain embodiments, within the scFv that binds NKG2D, the heavy chain variable domain is positioned at the N-terminus of the light chain variable domain. In certain embodiments, within each scFv that binds BAFF-R, the heavy chain variable domain is positioned at the N-terminus of the light chain variable domain. In certain embodiments, the Fab fragment that binds NKG2D is not positioned between an antigen-binding site and the Fc or the portion thereof. In certain embodiments, no Fab fragment that binds BAFF-R is positioned between an antigen-binding site and the Fc or the portion thereof.

In another aspect, provided herein is a protein comprising:
(a) a first antigen-binding site comprising a Fab fragment that binds NKG2D;
(b) a second antigen-binding site comprising a single-chain variable fragment (scFv) that binds B cell-activating factor receptor (BAFF-R); and
(c) an Fc domain comprising a first antibody constant domain and a second antibody constant domain that form a heterodimer that binds CD16,
wherein the scFv is linked to the N-terminus of the first antibody constant domain via a hinge, and the Fab is linked to the N-terminus of the second antibody constant domain.

In some embodiments, the hinge comprises Gly-Ser.

In some embodiments of a protein disclosed herein, the first antigen-binding site binds human NKG2D. In some embodiments, the first antigen-binding site that binds NKG2D comprises a VH comprising complementarity-determining region 1 (CDR1), complementarity-determining region 2 (CDR2), and complementarity-determining region 3 (CDR3) comprising the amino acid sequences of SEQ ID NOs: 81, 82, and 112, respectively; and a VL comprising CDR1, CDR2, and CDR3 comprising the amino acid sequences of SEQ ID NOs: 86, 77, and 87, respectively. In some embodiments, the first antigen-binding site that binds NKG2D comprises a VH comprising CDR1, CDR2, and CDR3 sequences represented by the amino acid sequences of SEQ ID NOs: 81, 82, and 97, respectively; and a VL comprising CDR1, CDR2, and CDR3 sequences represented by the amino acid sequences of SEQ ID NOs: 86, 77, and 87, respectively. In some embodiments, the first antigen-binding site that binds NKG2D comprises a VH comprising an amino acid sequence at least 90% identical to SEQ ID NO:95 and a VL comprising an amino acid sequence at least 90% identical to SEQ ID NO:85. In certain embodiments, the first antigen-binding site that binds NKG2D comprises a VH comprising an amino acid sequence of SEQ ID NO:95 and a VL comprising an amino acid sequence of SEQ ID NO:85.

In some embodiments of a protein disclosed herein, the second antigen-binding site comprises a heavy chain variable domain comprising CDR1, CDR2, and CDR3 sequences of SEQ ID NOs: 260, 249, and 261, respectively; and a light chain variable domain comprising CDR1, CDR2, and CDR3 sequences of SEQ ID NOs: 217, 77, and 259, respectively.

In some embodiments of a protein disclosed herein, the second antigen-binding site comprises a heavy chain variable domain comprising CDR1, CDR2, and CDR3 sequences of SEQ ID NOs: 214, 233, and 248, respectively; and a light chain variable domain comprising CDR1, CDR2, and CDR3 sequences of SEQ ID NOs: 217, 77, and 249, respectively. In some embodiments, the second antigen-binding site comprises a heavy chain variable domain at least 90% identical to SEQ ID NO:250 and a light chain variable domain at least 90% identical to SEQ ID NO:251.

In some embodiments of a protein disclosed herein, the second antigen-binding site comprises a VH with a G44C substitution relative to SEQ ID NO:250, and a VL with a G100C substitution relative to SEQ ID NO:251. In some embodiments, the second antigen-binding site comprises a VH comprising the amino acid sequence of SEQ ID NO:252 and a VL comprising the amino acid sequence of SEQ ID NO:253, or a VH comprising the amino acid sequence of SEQ ID NO:250 and a VL comprising the amino acid sequence of SEQ ID NO:251. In some embodiments, the second antigen-binding site comprises a VH comprising the amino acid sequence of SEQ ID NO:252 and a VL comprising the amino acid sequence of SEQ ID NO:253. In some embodiments, the second antigen-binding site comprises a VH comprising the amino acid sequence of SEQ ID NO:250 and a VL comprising the amino acid sequence of SEQ ID NO:251.

In some embodiments of a protein disclosed herein, the second antigen-binding site comprises a single-chain fragment variable (scFv), and the scFv comprises a VH comprising the amino acid sequence of SEQ ID NO:252 and a VL comprising the amino acid sequence of SEQ ID NO:253. In some embodiments, the second antigen-binding site comprises an scFv and the the scFv comprises an amino acid sequence at least 90% identical to a sequence selected from the group consisting of SEQ ID NOs: 254 and 255. In some embodiments, the second antigen-binding site comprises an scFv and the scFv comprises an amino acid sequence at least 90% identical to SEQ ID NO:254. In some embodiments, the second antigen-binding site comprises an scFv and the scFv comprises an amino acid sequence of SEQ ID NO:254.

In some embodiments of a protein disclosed herein, the protein comprises an amino acid sequence at least 90% identical to SEQ ID NO:270. In some embodiments, the protein comprises an amino acid sequence of SEQ ID NO:270. In some embodiments, the protein comprises an amino acid sequence at least 90% identical to SEQ ID NO:271. In some embodiments, the protein comprises an amino acid sequence of SEQ ID NO:271.

In some embodiments of a protein disclosed herein, the second antigen-binding site binds human BAFF-R with a dissociation constant (K_{D}) smaller than or equal to 5 nM, as measured by surface plasmon resonance (SPR).

In some embodiments of a protein disclosed herein, the second antigen-binding site inhibits (e.g., blocks) binding of BAFF-R to BAFF (e.g., by at least 50%, at least 75%, at least 90%, at least 95% or at least 99% as measured in a competitive binding assay).

In another aspect, provided herein is a protein comprising:
(a) a first antigen-binding site comprising a VH and a VL of an anti-NKG2D antibody, wherein the VH comprises the amino acid sequence of SEQ ID NO:95 and the VL comprises the amino acid sequence of SEQ ID NO:85;
(b) a second antigen-binding site comprising a VH and a VL of an anti-BAFF-R antibody, wherein the VH comprises the amino acid sequence of SEQ ID NO:252 and the VL comprises the amino acid sequence of SEQ ID NO:253; and
(c) an antibody Fc domain or a portion thereof sufficient to bind CD16, or a third antigen-binding site that binds CD16.

In another aspect, provided herein is a protein comprising:
(a) a first antigen-binding site comprising a VH and a VL of an anti-NKG2D antibody, wherein the VH comprises the amino acid sequence of SEQ ID NO:95 and the VL comprises the amino acid sequence of SEQ ID NO:85;
(b) a second antigen-binding site comprising the amino acid sequence of SEQ ID NO:254; and
(c) an antibody Fc domain or a portion thereof sufficient to bind CD16, or a third antigen-binding site that binds CD16.

In some embodiments of a protein disclosed herein, the antibody Fc domain is a human IgG1 antibody Fc domain. In some embodiments, the antibody Fc domain or the portion thereof comprises an amino acid sequence at least 90% identical to SEQ ID NO:118. In certain embodiments, at least one polypeptide chain of the antibody Fc domain comprises one or more mutations, relative to SEQ ID NO:118, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system. In certain embodiments, at least one polypeptide chain of the antibody Fc domain comprises one or more mutations, relative to SEQ ID NO:118, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, F405L, Y407A, Y407I, Y407V, K409F, K409W, K409D, K409R, T411D, T411E, K439D, and K439E, numbered according to the EU numbering system. In certain embodiments, one polypeptide chain of the antibody heavy chain constant region comprises one or more mutations, relative to SEQ ID NO:118, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:118, at one or more positions selected from Q347, Y349, L351, S354, E356, E357, S364, T366, L368, K370, N390, K392, T394, D399, D401, F405, Y407, K409, T411, and K439, numbered according to the EU numbering system. In certain embodiments, one polypeptide chain of the antibody heavy chain constant region comprises K360E and K409W substitutions relative to SEQ ID NO:118; and the other polypeptide chain of the antibody heavy chain constant region comprises Q347R, D399V and F405T substitutions relative to SEQ ID NO:118, numbered according to the EU numbering system. In certain embodiments, one polypeptide chain of the antibody heavy chain constant region comprises an F405L substitution relative to SEQ ID NO:118; and the other polypeptide chain of the antibody heavy chain constant region comprises a K409R substitution relative to SEQ ID NO:118, numbered according to the EU numbering system. In certain embodiments, one polypeptide chain of the antibody heavy chain constant region comprises a Y349C substitution relative to SEQ ID NO:118; and the other polypeptide chain of the antibody heavy chain constant region comprises an S354C substitution relative to SEQ ID NO:118, numbered according to the EU numbering system.

In another aspect, the present application provides a protein comprising:
(a) a first polypeptide comprising the amino acid sequence of SEQ ID NO:270;
(b) a second polypeptide comprising the amino acid sequence of SEQ ID NO:194; and
(c) a third polypeptide comprising the amino acid sequence of SEQ ID NO:195.

In another aspect, the present application provides a protein comprising:
(a) a first polypeptide comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:270;
(b) a second polypeptide comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:194; and
(c) a third polypeptide comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:195.

In some embodiments, the protein provided herein comprises:
(a) a first polypeptide comprising an amino acid sequence at least 90% identical to the amino acid sequence of SEQ ID NO:270;
(b) a second polypeptide comprising an amino acid sequence at least 90% identical to the amino acid sequence of SEQ ID NO:194; and
(c) a third polypeptide comprising an amino acid sequence at least 90% identical to the amino acid sequence of SEQ ID NO:195.

In some embodiments, the protein provided herein comprises:
(a) a first polypeptide comprising an amino acid sequence at least 95% identical to the amino acid sequence of SEQ ID NO:270;
(b) a second polypeptide comprising an amino acid sequence at least 95% identical to the amino acid sequence of SEQ ID NO:194; and
(c) a third polypeptide comprising an amino acid sequence at least 95% identical to the amino acid sequence of SEQ ID NO:195.

In some embodiments, the protein provided herein comprises:
(a) a first polypeptide comprising an amino acid sequence at least 99% identical to the amino acid sequence of SEQ ID NO:270;
(b) a second polypeptide comprising an amino acid sequence at least 99% identical to the amino acid sequence of SEQ ID NO:194; and
(c) a third polypeptide comprising an amino acid sequence at least 99% identical to the amino acid sequence of SEQ ID NO:195.

In some embodiments, the protein provided herein comprises:
(a) a first polypeptide comprising an amino acid sequence at least 90% identical to the amino acid sequence of SEQ ID NO:270;
(b) a second polypeptide comprising the amino acid sequence of SEQ ID NO:194; and
(c) a third polypeptide comprising the amino acid sequence of SEQ ID NO:195.

In some embodiments, the protein provided herein comprises:
(a) a first polypeptide comprising an amino acid sequence at least 95% identical to the amino acid sequence of SEQ ID NO:270;
(b) a second polypeptide comprising the amino acid sequence of SEQ ID NO:194; and
(c) a third polypeptide comprising the amino acid sequence of SEQ ID NO:195.

In some embodiments, the protein provided herein comprises:
(a) a first polypeptide comprising an amino acid sequence at least 99% identical to the amino acid sequence of SEQ ID NO:270;
(b) a second polypeptide comprising the amino acid sequence of SEQ ID NO:194; and
(c) a third polypeptide comprising the amino acid sequence of SEQ ID NO:195.

In some embodiments, the protein provided herein comprises a polypeptide comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:270. In some embodiments, the protein provided herein comprises a polypeptide comprising an amino acid sequence at least 95% identical to the amino acid sequence of SEQ ID NO:270. In some embodiments, the protein provided herein comprises a polypeptide comprising an amino acid sequence at least 99% identical to the amino acid sequence of SEQ ID NO:270.

In some embodiments, the protein provided herein comprises a polypeptide comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:194. In some embodiments, the protein provided herein comprises a polypeptide comprising an amino acid sequence at least 95% identical to the amino acid sequence of SEQ ID NO:194. In some embodiments, the protein provided herein comprises a polypeptide comprising an amino acid sequence at least 99% identical to the amino acid sequence of SEQ ID NO:194.

In some embodiments, the protein provided herein comprises a polypeptide comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:195. In some embodiments, the protein provided herein comprises a polypeptide comprising an amino acid sequence at least 95% identical to the amino acid sequence of SEQ ID NO:195. In some embodiments, the protein provided herein comprises a polypeptide comprising an amino acid sequence at least 99% identical to the amino acid sequence of SEQ ID NO:195.

In another aspect, the present application provides a protein comprising:
(a) a first polypeptide comprising the amino acid sequence of SEQ ID NO:271;
(b) a second polypeptide comprising the amino acid sequence of SEQ ID NO:272; and
(c) a third polypeptide comprising the amino acid sequence of SEQ ID NO:273.

In another aspect, the present application provides a pharmaceutical composition comprising a protein disclosed herein and a pharmaceutically acceptable carrier.

In another aspect, the present application provides a cell comprising one or more nucleic acids encoding a protein disclosed herein.

In another aspect, the present application provides a method of enhancing tumor cell death, the method comprising exposing the tumor cell and a natural killer cell to an effective amount of a protein disclosed herein or a pharmaceutical composition disclosed herein.

In another aspect, the present application provides a method of treating cancer, the method comprising administering to a subject in need thereof an effective amount of a protein disclosed herein or a pharmaceutical composition disclosed herein. In some embodiments, the cancer is selected from the group consisting of B-cell non-Hodgkin's lymphoma (B-NHL), chronic lymphocytic leukemia (CLL), mantle cell lymphoma (MCL), follicular lymphoma (FL), diffuse large B-cell lymphoma (DLBCL), marginal zone lymphoma, mucosa-associated lymphoid tissue (MALT) lymphoma, primary mediastinal B-cell lymphoma, and acute lymphocytic leukemia (ALL).

In another aspect, the present application provides a method of enhancing B cell death, the method comprising exposing the B cell and a natural killer cell to an effective amount of a protein disclosed herein or a pharmaceutical composition disclosed herein.

In another aspect, the present application provides a method of treating an autoimmune inflammatory disease, the method comprising administering to a subject in need thereof an effective amount of a protein disclosed herein or a pharmaceutical composition disclosed herein.

In some embodiments of a protein disclosed herein, the protein is a purified protein. In some embodiments, the protein is purified using a method selected from the group consisting of: centrifugation, depth filtration, cell lysis, homogenization, freeze-thawing, affinity purification, gel filtration, ion exchange chromatography, hydrophobic interaction exchange chromatography, and mixed-mode chromatography.

### BRIEF DESCRIPTION OF THE DRAWINGS

FIG. 1 is a representation of a heterodimeric, multispecific antibody, e.g., a trispecific binding protein (TriNKET). Each arm can represent either the NKG2D binding domain, or the BAFF-R binding domain. In some embodiments, the NKG2D binding domain and the BAFF-R binding domains can share a common light chain.

FIG. 2A-FIG. 2E illustrate five exemplary formats of a multispecific binding protein, e.g., a trispecific binding protein (TriNKET). As shown in FIG. 2A, either the NKG2D-binding domain or the BAFF-R binding domain can take the scFv format (left arm). An antibody that contains a NKG2D targeting scFv, a BAFF-R targeting Fab fragment, and a heterodimerized antibody constant region is referred herein as the F3-TriNKET. An antibody that contains a BAFF-R targeting scFv, a NKG2D targeting Fab fragment, and a heterodimerized antibody constant region/domain that binds CD16 is referred herein as the F3′-TriNKET (FIG. 2E). As shown in FIG. 2B, both the NKG2D binding domain and BAFF-R binding domain can take the scFv format. FIG. 2C-FIG. 2D are illustrations of an antibody with three antigen-binding sites, including two antigen-binding sites that bind BAFF-R, and the NKG2D-binding site fused to the heterodimerized antibody constant region. These antibody formats are referred herein as F4-TriNKET. FIG. 2C illustrates that the two BAFF-R binding sites are in the Fab fragment format, and the NKG2D binding site in the scFv format. FIG. 2D illustrates that the BAFF-R binding sites are in the scFv format, and the NKG2D binding site is in the scFv format. FIG. 2E represents a trispecific antibody (TriNKET) that contains a BAFF-R targeting scFv, a NKG2D targeting Fab fragment, and a heterodimerized antibody constant region/domain (“CD domain”) that binds CD16. The antibody format is referred herein as F3′-TriNKET. In certain exemplary multispecific binding proteins, heterodimerization mutations on the antibody constant region include K360E and K409W on one constant domain; and Q347R, D399V and F405T on the opposite constant domain (shown as a triangular lock-and-key shape in the CD domains). The bold bar between the heavy and the light chain variable domains of the Fab fragments represents a disulfide bond.

FIG. 3 is a representation of a TriNKET in the Triomab form, which is a trifunctional, bispecific antibody that maintains an IgG-like shape. This chimera consists of two half antibodies, each with one light and one heavy chain, that originate from two parental antibodies. Triomab form may be a heterodimeric construct containing ½ of rat antibody and ½ of mouse antibody.

FIG. 4 is a representation of a TriNKET in the KiH Common Light Chain form, which involves the knobs-into-holes (KIHs) technology. KiH is a heterodimer containing 2 Fab fragments binding to target 1 and 2, and an Fc stabilized by heterodimerization mutations. TriNKET in the KiH format may be a heterodimeric construct with 2 Fab fragments binding to target 1 and target 2, containing two different heavy chains and a common light chain that pairs with both heavy chains.

FIG. 5 is a representation of a TriNKET in the dual-variable domain immunoglobulin (DVD-Ig™) form, which combines the target-binding domains of two monoclonal antibodies via flexible naturally occurring linkers, and yields a tetravalent IgG-like molecule. DVD-Ig™ is a homodimeric construct where variable domain targeting antigen 2 is fused to the N-terminus of a variable domain of Fab fragment targeting antigen 1. DVD-Ig™ form contains normal Fc.

FIG. 6 is a representation of a TriNKET in the Orthogonal Fab fragment interface (Ortho-Fab) form, which is a heterodimeric construct that contains 2 Fab fragments binding to target 1 and target 2 fused to Fc. Light chain (LC)-heavy chain (HC) pairing is ensured by orthogonal interface. Heterodimerization is ensured by mutations in the Fc.

FIG. 7 is a representation of a TriNKET in the 2-in-1 Ig format.

FIG. 8 is a representation of a TriNKET in the ES form, which is a heterodimeric construct containing two different Fab fragments binding to target 1 and target 2 fused to the Fc. Heterodimerization is ensured by electrostatic steering mutations in the Fc.

FIG. 9 is a representation of a TriNKET in the Fab Arm Exchange form: antibodies that exchange Fab fragment arms by swapping a heavy chain and attached light chain (half-molecule) with a heavy-light chain pair from another molecule, resulting in bispecific antibodies. Fab Arm Exchange form (cFae) is a heterodimer containing 2 Fab fragments binding to target 1 and 2, and an Fc stabilized by heterodimerization mutations.

FIG. 10 is a representation of a TriNKET in the SEED Body form, which is a heterodimer containing 2 Fab fragments binding to target 1 and 2, and an Fc stabilized by heterodimerization mutations.

FIG. 11 is a representation of a TriNKET in the LuZ-Y form, in which a leucine zipper is used to induce heterodimerization of two different HCs. The LuZ-Y form is a heterodimer containing two different scFabs binding to target 1 and 2, fused to Fc. Heterodimerization is ensured through leucine zipper motifs fused to C-terminus of Fc.

FIG. 12 is a representation of a TriNKET in the Cov-X-Body form.

FIG. 13A and FIG. 13B are representations of TriNKETs in the κλ-Body forms, which are heterodimeric constructs with two different Fab fragments fused to Fc stabilized by heterodimerization mutations: one Fab fragment targeting antigen 1 contains kappa LC, and the second Fab fragment targeting antigen 2 contains lambda LC. FIG. 13A is an exemplary representation of one form of a κλ-Body; FIG. 13B is an exemplary representation of another κλ-Body.

FIG. 14 is a representation of an OAsc-Fab heterodimeric construct that includes Fab fragment binding to target 1 and scFab binding to target 2, both of which are fused to the Fc domain. Heterodimerization is ensured by mutations in the Fc domain.

FIG. 15 is a representation of a DuetMab, which is a heterodimeric construct containing two different Fab fragments binding to antigens 1 and 2, and an Fc that is stabilized by heterodimerization mutations. Fab fragments 1 and 2 contain differential S-S bridges that ensure correct light chain and heavy chain pairing.

FIG. 16 is a representation of a CrossmAb, which is a heterodimeric construct with two different Fab fragments binding to targets 1 and 2, and an Fc stabilized by heterodimerization mutations. CL and CH1 domains, and VH and VL domains are switched, e.g., CH1 is fused in-line with VL, and CL is fused in-line with VH.

FIG. 17 is a representation of a Fit-Ig, which is a homodimeric construct where Fab fragment binding to antigen 2 is fused to the N-terminus of HC of Fab fragment that binds to antigen 1. The construct contains wild-type Fc.

FIG. 18A-FIG. 18C are line graphs showing binding of BAFF-R-targeting TriNKETs derived from hCOH-2 (FIG. 18A), Genentech Hu9.1-73 (FIG. 18B), and ianalumab-based antigen-binding site (the three versions, F3′, 2-Fab, and ianalumab-mAb, do not contain antibody-dependent cellular cytotoxicity-enhancing mutations present in the commercial ianalumab antibody) (FIG. 18C) to BAFF-R-positive RAJI cells.

FIG. 19A-FIG. 19C are line graphs showing NK cell-mediated lysis of BAFF-R-positive RAJI cells by primary NK cells in the presence of BAFF-R-targeting TriNKETs derived from hCOH-2 (FIG. 19A), Genentech Hu9.1-73 (FIG. 19B), and ianalumab-based antigen-binding site (the three versions, F3′, 2-Fab, and ianalumab-mAb, do not contain antibody-dependent cellular cytotoxicity-enhancing mutations present in the commercial ianalumab antibody) (FIG. 19C).

FIG. 20A-FIG. 20C are line graphs showing NK cell-mediated lysis of BAFF-R-positive RAJI cells by KHYG-CD16V cells in the presence of BAFF-R-targeting TriNKETs derived from hCOH-2 (FIG. 20A), Genentech Hu9.1-73 (FIG. 20B), and ianalumab-based antigen-binding site (the three versions, F3′, 2-Fab, and ianalumab-mAb, do not contain antibody-dependent cellular cytotoxicity-enhancing mutations present in the commercial ianalumab antibody) (FIG. 20C).

FIG. 21 is a graph showing fluorescence outputs from a blocking assay of BAFF-biotin binding to hBAFF-R expressed on CHO cells by the indicated antibodies.

FIG. 22A-FIG. 22D are graphs of fluorescence outputs from binding assays on CHO cells showing binding of indicated antibodies to hBAFF-R (FIG. 22A, FIG. 22B) or blocking assays of BAFF-biotin binding to BAFF-R by indicated antibodies (FIG. 22C, FIG. 22D).

FIG. 23A-FIG. 23E are flow cytometry plots showing binding of AB0369scFv expressed on yeast to no antigen control (FIG. 23A), h-BAFF-R-hFc (FIG. 23B), Irrelevant-hFc (FIG. 23C), hBAFF-R-GST (FIG. 23D), or Irrelevant-GST (FIG. 23E). Vertical axes indicates scFv expression as measured by detection of the Flag epitope tag; horizontal axes indicate binding of biotinylated control of BAFF-R constructs to scFv as measured by detection of streptavidin-PE.

FIG. 24A and FIG. 24B are graphs showing binding of AB0369 or indicated controls to human (FIG. 24A) or cynomolgus monkey (FIG. 24B) BAFF-R.

FIG. 25A-FIG. 25G detail a poly-specificity assay of a multi-specific binding proteins with a BAFF-R binding site derived from AB0369. FIG. 25A is a schematic of the assay. FIG. 25B-FIG. 25G show graphs of AB0369 (left panels), trastuzumab negative control (middle panels), or ixekizumab positive control (right panels) in the absence (top panels) or presence (bottom panels) of poly-specificity reagent (PSR).

FIG. 26 is a graph showing a KHYG-1-CD16aV cytotoxicity assay of Ramos cells as induced by a multispecific binding protein with a BAFF-R binding site derived from AB0369.

FIG. 27 is a graph showing fluorescence outputs from a binding assay showing blockage of BAFF-biotin binding to human BAFF-R expressed on CHO cells by AB0369 or indicated.

FIG. 28A-FIG. 28D are flow cytometry plots showing binding of hBAFF-R-hFc-His to parental AB0369scFv or clones selected from a library produced by affinity maturation expressed on yeast following successive rounds of selection. FIG. 28A shows binding to parental AB0369scFv; FIG. 28B shows binding to sample from the first round of clone selection; FIG. 28C shows binding to sample from the second round of clone selection; FIG. 28D shows binding to output from the second round of clone selection.

FIG. 29A-FIG. 29E are flow cytometry plots showing binding of hBAFF-R-hFc-His to AB0369 and affinity-matured scFv clones expressed on yeast. FIG. 29A shows binding to parental AB0369; FIG. 29B shows binding to AB0605; FIG. 29C shows binding to AB0622; FIG. 29D shows binding to AB0622; and FIG. 29E shows binding to ianalumab-based antigen-binding site.

FIG. 30A-FIG. 30C are graphs demonstrating BAFF-R binding and cytotoxicity of multi-specific binding proteins developed from affinity maturation of AB0369. FIG. 30A is a graph showing binding of multi-specific binding proteins with BAFF-R binding sites derived from indicated clones to human BAFF-R expressed on CHO cells. FIG. 30B is a graph showing a KHYG-1-CD16aV cytotoxicity assay of Ramos cells as induced by multi-specific binding proteins with BAFF-R binding sites derived from indicated clones. FIG. 30C is a graph showing a KHYG-1-CD16aV cytotoxicity assay of Ramos cells as induced by multi-specific binding proteins with BAFF-R binding sites derived from AB0622.

FIG. 31A-FIG. 31E detail a poly-specificity assay of multi-specific binding proteins with BAFF-R binding sites derived from AB00605 and AB0606. FIG. 31A is a schematic of the assay. FIG. 31B-FIG. 31E show graphs of AB0605 (left panels) or AB0606 (right panels) in the absence (top panels) or presence (bottom panels) of poly-specificity reagent (PSR).

FIG. 32A-FIG. 32C are flow cytometry plots showing binding of hBAFF-R-hFc-His to parental AB0369scFv or clones selected from a library produced by affinity maturation and expressed on yeast following successive rounds of selection. FIG. 32A shows binding to parental AB0369scFv; FIG. 32B shows binding to sample from the first round of clone selection;

FIG. 32C shows binding to sample from the second round of clone selection. FIG. 33A-FIG. 33E are flow cytometry plots showing binding of hBAFF-R-hFc-His to AB0369 and affinity-matured scFv clones expressed on yeast. FIG. 33A shows binding to parental AB0369; FIG. 33B shows binding to AB0679; FIG. 33C shows binding to AB0681; FIG. 33D shows binding to AB0682; and FIG. 33E shows binding to ianalumab-based antigen-binding site.

FIG. 34A-FIG. 34C are graphs demonstrating BAFF-R binding to multi-specific binding proteins developed from affinity maturation of AB0369. FIG. 34A is a graph showing binding of multi-specific binding proteins with BAFF-R binding sites derived from indicated clones to human BAFF-R expressed on CHO cells. FIG. 34B is a graph showing binding of multi-specific binding proteins with BAFF-R binding sites derived from indicated clones to cynomolgus monkey BAFF-R expressed on CHO cells. FIG. 34C is a graph showing fluorescence outputs from a binding assay showing blockage of BAFF-biotin binding to BAFF-R expressed on CHO cells by the indicated antibodies.

FIG. 35 is a graph showing a KHYG-1-CD16aV cytotoxicity assay of BJAB cells as induced by multi-specific binding proteins with BAFF-R binding sites derived from AB0679, AB0568, or Tool-F3′ positive control.

FIG. 36A-FIG. 36D are flow cytometry plots showing binding of hBAFF-R-hFc-His to parental AB0369scFv clones selected from a library produced by affinity maturation expressed on yeast following successive rounds of selection. FIG. 36A shows binding to parental AB0369scFv; FIG. 36B shows binding to sample from the first round of clone selection; FIG. 36C shows binding to sample from the second round of clone selection; and FIG. 36D shows binding to sample from the third round of clone selection.

FIG. 37A-FIG. 37F are flow cytometry plots showing binding of hBAFF-R-hFc-His to AB0369 and affinity-matured scFv clones expressed on yeast. FIG. 37A shows binding to parental AB0369; FIG. 37B shows binding to AB0682; FIG. 37C shows binding to AB0898; FIG. 37D shows binding to AB0899; FIG. 37E shows binding to AB0900; and FIG. 37F shows binding to ianalumab-based antigen-binding site.

FIG. 38 is a graph showing a KHYG-1-CD16aV cytotoxicity assay of BJAB cells as induced by multi-specific binding proteins with BAFF-R binding sites derived from AB0898, AB0899, or AB0900.

FIG. 39A-FIG. 39C show graphs of differential scanning calorimetry (DSC) profiles of AB0898 (FIG. 39A), AB0899 (FIG. 39B), and AB0900 (FIG. 39C).

FIG. 40 shows flow cytometry plots of binding of scFv clones expressed on yeast to biotinylated hBAFFR-Fc before (left) and after (right) challenge by incubation with 1 mM non-biotinylated hBAFFR-Fc.

FIG. 41A and FIG. 41B show flow cytometry plots of binding of scFv clones expressed on yeast to biotinylated hBAFFR-Fc before (FIG. 41A) and after (FIG. 41B) challenge by incubation with 1 mM non-biotinylated hBAFFR-Fc. Clones tested are (left-to-right) AB1080, AB1081, AB1084, AB1085, and ianalumab.

FIG. 42A and FIG. 42B are graphs showing binding of indicated antibody clones to human (FIG. 42A) or cynomolgus monkey (FIG. 42B) BAFF-R.

FIG. 43A-FIG. 431 detail a poly-specificity assay of a multi-specific binding proteins with a BAFF-R binding site derived from AB1080 or AB1081. FIG. 43A is a schematic of the assay. FIG. 43B-FIG. 431 show graphs of AB1080 (left panels), AB1081 (middle-left panels), trastuzumab negative control (middle-right panels), or ixekizumab positive control (right panels) in the absence (top panels) or presence (bottom panels) of poly-specificity reagent (PSR).

FIG. 44A and FIG. 44B show graphs of a KHYG-1-CD16aV cytotoxicity assay of BJAB cells as induced by multi-specific binding proteins with BAFF-R binding sites derived from AB1080 (FIG. 44A) or AB1085 (FIG. 44B) compared to Tool positive control.

FIG. 45 is a graph showing fluorescence outputs from a blocking assay of BAFF-biotin binding to human BAFF-R expressed on CHO cells by the indicated antibody clones.

FIG. 46A-FIG. 46D show graphs of nano-dual scanning fluorimetry (nanoDSF) analysis of multi-specific binding proteins with BAFF-R binding sites derived from AB1080 (FIG. 46A), AB1081 (FIG. 46B), AB1084 (FIG. 46C), and AB1085 (FIG. 46D).

FIG. 47 shows a graph of hydrophobic interaction chromatography (HIC) analysis of multi-specific binding proteins with BAFF-R binding sites derived from indicated antibodies.

FIG. 48 shows a graph of HIC analysis of AB1612 compared to indicated benchmark biologics.

FIG. 49A and FIG. 49B are graphs showing binding of indicated antibody clones to cynomolgus monkey (FIG. 49A) or human (FIG. 49B) BAFF-R.

FIG. 50 is a graph showing fluorescence outputs from a binding assay showing blockage of BAFF-biotin binding to human BAFF-R expressed on CHO cells by the indicated antibodies.

FIG. 51A-FIG. 51C show the surface charge distribution of the BAFF-R binding arm of AB1424/1612 F3′ TriNKET. Three orientations are shown: both façades (left panel: front view; center panel: back view) and the antigen-engaging surface (right panel: top view). The positively charged areas are colored blue, negatively charged areas red, and the hydrophobic surface white.

FIG. 52A-FIG. 52E are graphs showing evaluation of surface patches and CDRs length of the BAFF-R binding arm of AB1424/1612 F3′ TriNKET. Solid lines and corresponding arrows indicate the scoring of the BAFF-R binding arm of AB1424/1612 F3′ TriNKET in reference to a database of 377 late-stage therapeutic antibodies. In FIG. 52A and FIG. 52B, the two inner dashed lines indicate 2 standard deviations (>95% of reference molecules within this region), whereas the two outer most dashed lines indicate 3 standard deviations (>99.7% of reference molecules within this region). In each plot of FIG. 52C-FIG. 52E, there are two dashed lines—one closer and the other further to the solid line. The dashed line closer to the solid line indicates 2 standard deviations (>95% of reference molecules within this region), whereas the dashed line further to the solid line indicates 3 standard deviations (>99.7% of reference molecules within this region).

FIG. 53A-FIG. 53C show the surface charge distribution of the NKG2D binding arm of AB1424/1612 F3′ TriNKET. Three orientations are shown: both façades (left panel: front view; center panel: back view) and the antigen-engaging surface (right panel: top view). The positively charged areas are colored blue, negatively charged areas red, and the hydrophobic surface white.

FIG. 54A-FIG. 54E are graphs showing evaluation of surface patches and CDRs length of the NKG2D-R binding arm of AB1424/1612 F3′ TriNKET. Solid lines and corresponding arrows indicate the scoring of the BAFF-R binding arm of AB1424/1612 F3′ TriNKET in reference to a database of 377 late-stage therapeutic antibodies. In FIG. 54A and FIG. 54B, the two inner dashed lines indicate 2 standard deviations (>95% of reference molecules within this region), whereas the two outer most dashed lines indicate 3 standard deviations (>99.7% of reference molecules within this region). In each plot of FIG. 54C-FIG. 54E, there are two dashed lines—one closer and the other further to the solid line. The dashed line closer to the solid line indicates 2 standard deviations (>95% of reference molecules within this region), whereas the dashed line further to the solid line indicates 3 standard deviations (>99.7% of reference molecules within this region).

FIG. 55A and FIG. 55B are chromatograms showing HIC analysis of AB1424/1612 F3′ TriNKET (FIG. 55A) and comparison with adalimumab and pembrolizumab (FIG. 55B).

FIG. 56 is a graph showing capillary isoelectric focusing (cIEF) profiling of AB1424/1612 F3′ TriNKET.

FIG. 57A and FIG. 57B are graphs showing DSC profiling of AB1424/1612 F3′ TriNKET in PBS pH 7.4 (FIG. 57A) and HST pH 6.0 (FIG. 57B).

FIG. 58A and FIG. 58B are graphs showing n-curve analysis (FIG. 58A) and confidence interval (FIG. 58B) of AB1424/1612 F3′ TriNKET binding cell-based BAFF-R by Kinexa.

FIG. 59A and FIG. 59B are graphs showing binding of AB1424/1612 F3′ TriNKET and corresponding parental mAb to isogenic human (FIG. 59A) and cynomolgus (FIG. 59B) BAFF-R-CHO cells.

FIG. 60A-FIG. 60F are graphs showing binding of AB1424/1612 F3′ TriNKET to BAFF-R+ tumor cell lines. Titrations were done in the presence of BJAB (FIG. 60A), Raji (FIG. 60B), RL (FIG. 60C), Rs4;11 (FIG. 60D), Jeko-1 (FIG. 60E), SUDHL-6 cells (FIG. 60F). FOB=fold over background of stained vs. unstained samples.

FIG. 61A-FIG. 61H are graphs showing surface plasmon resonance (SPR) binding of AB1424/1612 F3′ TriNKET to human NKG2D. Colored lines represent raw data and black traces represent 1:1 binding fit (top panel). Corresponding steady state fits (bottom panel). The vertical line denotes steady state K_{D}.

FIG. 62A-FIG. 62H are graphs showing SPR binding of AB1424/1612 F3′ TriNKET to cynomolgus NKG2D. Colored lines represent raw data and black traces represent 1:1 binding fit (top panel). Corresponding steady state fits (bottom panel). The vertical line denotes steady state K_{D}.

FIG. 63A-FIG. 63H are graphs showing SPR binding of AB1424/1612 F3′ TriNKET to human CD16a V158 (top panels) or trastuzumab (bottom panels). Colored lines represent raw data and black traces represent 1:1 binding fit.

FIG. 64A-FIG. 64P are graphs showing SPR binding of AB1424/1612 F3′ TriNKET (top panels) or trastuzumab (bottom panels) to human CD16a F158. Colored lines represent raw data and black traces represent 1:1 binding fit (top panel).

FIG. 65A-FIG. 65H are graphs showing SPR binding of AB1424/1612 F3′ TriNKET to cynomolgus CD16. Colored lines represent raw data and black traces represent 1:1 binding fit (top panel). Corresponding steady state fits (bottom panel). The vertical line denotes steady state K_{D}.

FIG. 66 is a graph showing SPR binding of AB1424/1612 F3′ TriNKET to NKG2D (brown), CD16a (purple), or mixed CD16a and NKG2D (blue) surfaces.

FIG. 67A and FIG. 67B are sensorgram graphs representing binding of BAFF-R (800 nM) followed by binding of hNKG2D (7 μM) to captured AB1424/1612 F3′ TriNKET (FIG. 67A) or reverse order of target binding with human NKG2D (7 μM) followed by BAFF-R (800 nM) (FIG. 67B).

FIG. 68A and FIG. 68B are graphs showing SPR analysis of BAFF-R and TACI binding to immobilized AB1424/1612 F3′ TriNKET (FIG. 68A) and specific anti-TACI mAb (FIG. 68B).

FIG. 69A and FIG. 69B are graphs showing binding of AB1424/1612 F3′ TriNKET to parental cells not expressing BCMA (FIG. 69A) and isogenic BCMA+ cells compared to control mAb specific anti-BCMA (FIG. 69B).

FIG. 70A and FIG. 70B are graphs showing AB1424/1612 F3′ TriNKET binding to isogenic BAFFR+ CHO cells (FIG. 70A) and lack of reactivity with parental CHO line (FIG. 70B).

FIG. 71A-FIG. 71G detail a poly-specificity assay of a AB1424/1612 F3′ TriNKET. FIG. 71A is a schematic of the assay. FIG. 71B-FIG. 71G show graphs of AB1424/1612 F3′ TriNKET (left panels), trastuzumab negative control (middle panels), or ixekizumab positive control (right panels) in the absence (top panels) or presence (bottom panels) of poly-specificity reagent (PSR).

FIG. 72A-FIG. 72C show graphs of cytotoxicity assays of RL cells as induced by AB1424/1612 F3′ TriNKET (blue) or parental monoclonal antibody (red) using NK cells from three donors.

FIG. 73A-FIG. 73D show schematic representations of AB1424/1612 F3′ TriNKET and controls for elucidating mechanism of action.

FIG. 74 shows a graph of a KHYG-1-CD16aV cytotoxicity assay of BJAB cells as induced by AB1424/1612 F3′ TriNKET (blue), AB1424/1612 F3′ TriNKET lacking NKG2D binding (black), or AB1424/1612 F3′ TriNKET-Fc silenced (red), or palivizumab F3′ TriNKET (grey).

FIG. 75A-FIG. 75H are sensorgram graphs showing binding of AB1424/1612 F3′ TriNKET (top panels) and trastuzumab (bottom panels) to human CD64. Raw sensorgrams (colored) with 1:1 fitted curves overlaid (black).

FIG. 76A-FIG. 76H are sensorgram graphs showing binding of AB1424/1612 F3′ TriNKET (top panels) and trastuzumab (bottom panels) to cynomolgus monkey CD64. Raw sensorgrams (colored) with 1:1 fitted curves overlaid (black).

FIG. 77A-FIG. 77P are sensorgram graphs showing binding of AB1424/1612 F3′ TriNKET (FIG. 77A-FIG. 77H) and trastuzumab (FIG. 77I-FIG. 77P) to human CD32a H131. For each molecule the upper panel represents raw sensorgrams and the lower panel represents the steady state affinity fit.

FIG. 78A-FIG. 78P are sensorgram graphs showing binding of AB1424/1612 F3′ TriNKET (FIG. 78A-FIG. 78H) and trastuzumab (FIG. 78I-FIG. 78P) to human CD32a R131. For each molecule the upper panel represents raw sensorgrams and the lower panel represents the steady state affinity fit.

FIG. 79A-FIG. 79P are sensorgram graphs showing binding of AB1424/1612 F3′ TriNKET (FIG. 79A-FIG. 79H) and trastuzumab (FIG. 79I-FIG. 79P) to human CD32b. For each molecule the upper panel represents raw sensorgrams and the lower panel represents the steady state affinity fit.

FIG. 80A-FIG. 80P are sensorgram graphs showing binding of AB1424/1612 F3′ TriNKET (FIG. 80A-FIG. 80H) and trastuzumab (FIG. 80I-FIG. 80P) to human CD16b. For each molecule the upper panel represents raw sensorgrams and the lower panel represents the steady state affinity fit.

FIG. 81A-FIG. 81H are sensorgram graphs showing binding of AB1424/1612 F3′ TriNKET (top panels) and trastuzumab (bottom panels) to cynomolgus monkey CD16.

FIG. 82A-FIG. 82P are sensorgram graphs showing binding of AB1424/1612 F3′ TriNKET (FIG. 82A-FIG. 82H) and trastuzumab (FIG. 82I-FIG. 82P) to human FcRn at pH 6.0. For each molecule the upper panel represents raw sensorgrams and the lower panel represents the steady state affinity fit.

FIG. 83A-FIG. 83P are sensorgram graphs showing binding of AB1424/1612 F3′ TriNKET (FIG. 83A-FIG. 83H) and trastuzumab (FIG. 83I-FIG. 83P) to cynomolgus monkey FcRn at pH 6.0. For each molecule the upper panel represents raw sensorgrams and the lower panel represents the steady state affinity fit.

FIG. 84A-FIG. 84H are raw sensorgram graphs showing binding of AB1424/1612 F3′ TriNKET (top panels) and trastuzumab (bottom panels) to human (left panels) and cynomolgus monkey (right panels) FcRn at pH 7.4.

FIG. 85 shows a graph of a KHYG-1-CD16aV cytotoxicity assay of BJAB cells as induced by two lots of AB1424/1612 F3′ TriNKET (blue and red) or human IgG1k (grey).

FIG. 86A shows a graph of a KHYG-1-CD16aV cytotoxicity assay of BJAB cells as induced by two lots of AB1424/1612 F3′ TriNKET (blue and red) or human IgG1k (grey).

FIG. 86B shows a graph of a KHYG-1-CD16aV cytotoxicity assay of BJAB cells as induced by AB1424/1612 F3′ TriNKET at nominal drug concentrations (NDC) of 50% (red), 100% (blue), and 200% (green).

FIG. 87A and FIG. 87B show PEG precipitation Cm plots of AB1424/1612 F3′ TriNKET in histidine (FIG. 87A) and acetate (FIG. 87B).

FIG. 88A and FIG. 88B show PEG precipitation Cm plots of adalimumab in histidine (FIG. 88A) and acetate (FIG. 88B).

FIG. 89A-FIG. 89C show k_{D} plots of adalimumab in acetate (FIG. 89A), histidine (FIG. 89B), and phosphate (FIG. 89C).

FIG. 90A-FIG. 90C show k_{D} plots of AB1424/1612 F3′ TriNKET in acetate (FIG. 90A), histidine (FIG. 90B), and phosphate (FIG. 90C).

FIG. 91 is a viscosity vs. concentration plot of AB1424/1612 F3′ TriNKET at 25° C.

FIG. 92 is a chromatogram of size-exclusion chromatography (SEC) analysis of AB1424/1612 F3′ TriNKET after 4 weeks at 40° C. in HST, pH 6.0 compared to control.

FIG. 93 is a graph of capillary electrophoresis sodium dodecyl sulfate (CE-SDS) analysis of AB1424/1612 F3′ TriNKET after 4 weeks at 40° C. in HST, pH 6.0 compared to control.

FIG. 94 is a graph showing cIEF profiling of AB1424/1612 F3′ TriNKET in HST, pH 6.0 compared to control.

FIG. 95A-FIG. 95C show binding of AB1424/1 612 F3′ TriNKET to hBAFF-R, hNKG2D and hCD16aV after 4 weeks at 40° C. in HST, pH 6.0 compared to control. FIG. 95A is a graph showing binding to BJAB cells (BAFF-R); FIG. 95B is a sensorgram showing binding to hNKG2D by SPR. FIG. 95C is a sensorgram showing binding to hCD16a V158 by SPR. Colored sensorgrams represent raw data and black overlays represent the kinetic fit of the raw data.

FIG. 96 shows a graph of a KHYG-1-CD16aV cytotoxicity assay of BJAB cells as induced by AB1424/1612 F3′ TriNKET after 1 week (red), 2 weeks (green), 3 weeks (purple) at 40° C. in HST, pH 6.0 compared to control (blue).

FIG. 97A-FIG. 97C show the surface charge distribution of the BAFF-R binding arm of AB1424/1612 F4 TriNKET. Three orientations are shown: both façades (left panel: front view; center panel: back view) and the antigen-engaging surface (right panel: top view). The positively charged areas are colored blue, negatively charged areas red, and the hydrophobic surface white.

FIG. 98A-FIG. 98E are graphs showing evaluation of surface patches and CDRs length of the BAFF-R binding arm of AB1424/1612 F4 TriNKET. Solid lines and corresponding arrows indicate the scoring of the BAFF-R binding arm of AB1424/1612 F4 TriNKET in reference to a database of 377 late-stage therapeutic antibodies. In FIG. 98A and FIG. 98B, the two inner dashed lines indicate 2 standard deviations (>95% of reference molecules within this region), whereas the two outer most dashed lines indicate 3 standard deviations (>99.7% of reference molecules within this region). In each plot of FIG. 98C-FIG. 98E, there are two dashed lines—one closer and the other further to the solid line. The dashed line closer to the solid line indicates 2 standard deviations (>95% of reference molecules within this region), whereas the dashed line further to the solid line indicates 3 standard deviations (>99.7% of reference molecules within this region).

FIG. 99A-FIG. 99C shows the surface charge distribution of the NKG2D binding arm of AB1424/1612 F4 TriNKET. Three orientations are shown: both façades (left panel: front view; center panel: back view) and the antigen-engaging surface (right panel: top view). The positively charged areas are colored blue, negatively charged areas red, and the hydrophobic surface white.

FIG. 100A-FIG. 100E are graphs showing evaluation of surface patches and CDRs length of the NKG2D-R binding arm of AB1424/1612 F4 TriNKET. Solid lines and corresponding arrows indicate the scoring of the BAFF-R binding arm of AB1424/1612 F3′ TriNKET in reference to a database of 377 late-stage therapeutic antibodies. In FIG. 100A and FIG. 100B, the two inner dashed lines indicate 2 standard deviations (>95% of reference molecules within this region), whereas the two outer most dashed lines indicate 3 standard deviations (>99.7% of reference molecules within this region). In each plot of FIG. 100C-FIG. 100E, there are two dashed lines—one closer and the other further to the solid line. The dashed line closer to the solid line indicates 2 standard deviations (>95% of reference molecules within this region), whereas the dashed line further to the solid line indicates 3 standard deviations (>99.7% of reference molecules within this region).

FIG. 101A-FIG. 101C are chromatograms of SEC analysis of three lots of AB1424/1612 F4 TriNKET.

FIG. 102 is a graph showing cIEF profiling of three lots of AB1424/1612 F4 TriNKET.

FIG. 103A and FIG. 103B. FIG. 103A is a graph of HIC analysis of AB1424/1612 F4 TriNKET compared in indicated benchmark commercial antibodies. FIG. 103B is a graph of thermal stability analysis of AB1424/1612 F4 TriNKET by DSC.

FIG. 104A and FIG. 104B show extracted ion chromatogram (XICs) for the engineered disulfide pair in the Fc (non-reduced and reduced) and the most intense charge state for that peptide pair.

FIG. 105A and FIG. 105B show XICs for the engineered disulfide pair in the scFv (non-reduced and reduced) and the most intense charge state for that peptide pair.

FIG. 106A and FIG. 106B are graphs showing binding of AB1424/1612 F4 TriNKET, parental mAb, and F4-palivizumab to human (FIG. 106A) and cynomolgus (FIG. 106B) BAFF-R+ isogenic CHO cells.

FIG. 107A-FIG. 107L are sensorgram graphs of SPR binding of AB1424/1612 F4 TriNKET to human NKG2D.

FIG. 108A-FIG. 108P are sensorgram graphs showing binding of AB1424/1612 F4 TriNKET (FIG. 108A-FIG. 108H) and trastuzumab (FIG. 108I-FIG. 108P) to human CD32a R131. For each molecule the upper panel represents raw sensorgrams and the lower panel represents the steady state affinity fit.

FIG. 109A-FIG. 109H are sensorgram graphs showing binding of AB1424/1612 F4 TriNKET (top panels) and trastuzumab (bottom panels) to human CD16a V158. For each molecule the upper panel represents raw sensorgrams and the lower panel represents the steady state affinity fit.

FIG. 110A-FIG. 110H are sensorgram graphs showing binding of AB1424/1612 F4 TriNKET (top panels) and trastuzumab (bottom panels) to human CD16a V158. For each molecule the upper panel represents raw sensorgrams and the lower panel represents the steady state affinity fit.

FIG. 111A-FIG. 111H are sensorgram graphs showing binding of AB1424/1612 F4 TriNKET (top panels) and trastuzumab (bottom panels) to human CD64. Raw sensorgrams (colored) with 1:1 fitted curves overlaid (black).

FIG. 112A-FIG. 112H are sensorgram graphs showing binding of AB1424/1612 F4 TriNKET (top panels) and trastuzumab (bottom panels) to cynomolgus CD64. Raw sensorgrams (colored) with 1:1 fitted curves overlaid (black).

FIG. 113A-FIG. 113P are sensorgram graphs showing binding of AB1424/1612 F4 TriNKET (FIG. 113A-FIG. 113H) and trastuzumab (FIG. 113I-FIG. 113P) to human CD32a H131. For each molecule the upper panel represents raw sensorgrams and the lower panel represents the steady state affinity fit.

FIG. 114A-FIG. 114P are sensorgram graphs showing binding of AB1424/1612 F4 TriNKET (FIG. 114A-FIG. 114H) and trastuzumab (FIG. 114I-FIG. 114P) to human CD32b. For each molecule the upper panel represents raw sensorgrams and the lower panel represents the steady state affinity fit.

FIG. 115A-FIG. 115P are sensorgram graphs showing binding of AB1424/1612 F4 TriNKET (FIG. 115A-FIG. 115H) and trastuzumab (FIG. 115I-FIG. 115P) to human CD16b. For each molecule the upper panel represents raw sensorgrams and the lower panel represents the steady state affinity fit.

FIG. 116A-FIG. 116P are sensorgram graphs showing binding of AB1424/1612 F4 TriNKET (FIG. 116A-FIG. 116H) and trastuzumab (FIG. 116I-FIG. 116P) to human FcRn at pH 6.0. For each molecule the upper panel represents raw sensorgrams and the lower panel represents the steady state affinity fit.

FIG. 117A-FIG. 117P are sensorgram graphs showing binding of AB1424/1612 F4 TriNKET (FIG. 117A-FIG. 117H) and trastuzumab (FIG. 117I-FIG. 117P) to cynomolgus FcRn at pH 6.0. For each molecule the upper panel represents raw sensorgrams and the lower panel represents the steady state affinity fit.

FIG. 118A-FIG. 118H are raw sensorgram graphs showing binding of AB1424/1612 F4 TriNKET (top panels) and trastuzumab (bottom panels) to human (left panels) and cynomolgus (right panels) FcRn at pH 7.4.

FIG. 119 is a graph showing SPR binding of AB1424/1612 F4 TriNKET to NKG2D (brown), CD16a (purple), or mixed CD16a and NKG2D (blue) surfaces.

FIG. 120A and FIG. 120B are graphs showing sequential saturation of BAFF-R and NKG2D by AB1424/1612 F4 TriNKET.

FIG. 121A-FIG. 121I detail a poly-specificity assay of AB1424/1612 F4 TriNKET. FIG. 121A is a schematic of the assay. FIG. 121B-FIG. 121I show graphs of AB1424/1612 F4 TriNKET (left panels), trastuzumab (center-left panels), rituximab (center-right panels), or ixekizumab (right panels) in the absence (top panels) or presence (bottom panels) of poly-specificity reagent (PSR).

FIG. 122 is a graph of a KHYG-1-CD16aV cytotoxicity assay of BJAB cells as induced by AB1424/1612 F4 TriNKET (blue) and human IgG1k (grey).

FIG. 123 is a graph of a rested hNK-induced cytotoxicity assay of BJAB cells as induced by AB1424/1612 F4 TriNKET (blue) and parental mAb (red).

FIG. 124 is a chromatogram of SEC analysis of AB1424/1612 F4 TriNKET after 4 weeks at 40° C. in HST, pH 6.0 compared to control.

FIG. 125 is a graph showing reduced CE-SDS analysis of AB1424/1612 F4 TriNKET after 4 weeks at 40° C. in HST, pH 6.0 compared to control.

FIG. 126 is a graph showing cIEF profiling of AB1424/1612 F4 TriNKET after 4 weeks at 40° C. in HST, pH 6.0 compared to control.

FIG. 127 is a graph showing binding of AB1424/1612 F4 TriNKET to hBAFF-R+ cells after 4 weeks at 40° C. in HST, pH 6.0 compared to control.

FIG. 128A and FIG. 128B are sensorgram graphs showing SPR binding of hCD16aV to AB1424/1612 F4 TriNKET after 4 weeks at 40° C. in HST, pH 6.0 (FIG. 128B) compared to control (FIG. 128A).

FIG. 129 is a graph of a KHYG-1-CD16aV cytotoxicity assay of BJAB cells as induced by AB1424/1612 F4 TriNKET after 4 weeks at 40° C. in HST, pH 6.0 (red) compared to control (blue).

FIG. 130 is a chromatogram of SEC analysis of AB1424/1612 F4 TriNKET after forced oxidation compared to control.

FIG. 131 is a graph showing reduced CE-SDS analysis of AB1424/1612 F4 TriNKET after forced oxidation compared to control.

FIG. 132 is a graph showing binding of AB1424/1612 F4 TriNKET to hBAFF-R+ cells after forced oxidation.

FIG. 133A and FIG. 133B are sensorgram graphs showing SPR binding of hCD16aV to AB1424/1612 F4 TriNKET control (FIG. 133A) and after forced oxidation (FIG. 133B).

FIG. 134 is a graph of a KHYG-1-CD16aV cytotoxicity assay of BJAB cells as induced by AB1424/1612 F4 TriNKET after forced oxidation (red) and control (blue).

FIG. 135 is a chromatogram of SEC analysis of AB1424/1612 F4 TriNKET after long term low pH stress compared to control.

FIG. 136 is a graph showing reduced CE-SDS analysis of AB1424/1612 F4 TriNKET after long term low pH stress compared to control.

FIG. 137 is a graph showing cIEF profiling of AB1424/1612 F4 TriNKET after long term low pH stress compared to control.

FIG. 138 is a graph showing binding of AB1424/1612 F4 TriNKET to hBAFF-R+ cells after long term low pH stress compared to control.

FIG. 139A and FIG. 139B are sensorgram graphs showing SPR binding of hCD16aV to AB1424/1612 F4 TriNKET after long term low pH stress (FIG. 139B) compared to control (FIG. 139A).

FIG. 140 is a graph of a KHYG-1-CD16aV cytotoxicity assay of BJAB cells as induced by AB1424/1612 F4 TriNKET after long term low pH stress (red) and control (blue).

FIG. 141 is a chromatogram of SEC analysis of AB1424/1612 F4 TriNKET after long term high pH stress compared to control.

FIG. 142 is a graph showing reduced CE-SDS analysis of AB1424/1612 F4 TriNKET after long term high pH stress compared to control.

FIG. 143 is a graph showing cIEF profiling of AB1424/1612 F4 TriNKET after long term high pH stress compared to control.

FIG. 144 is a graph showing binding of AB1424/1612 F4 TriNKET to hBAFF-R+ cells after long term high pH stress (red) compared to control (blue).

FIG. 145A and FIG. 145B are sensorgram graphs showing SPR binding of hCD16aV to AB1424/1612 F4 TriNKET after long term high pH stress (FIG. 145B) compared to control (FIG. 145A).

FIG. 146 is a graph of a KHYG-1-CD16aV cytotoxicity assay of BJAB cells as induced by AB1424/1612 F4 TriNKET after long term high pH stress (red) and control (blue).

FIG. 147 is a chromatogram of SEC analysis of AB1424/1612 F4 TriNKET after 6 freeze/thaw cycles compared to control.

FIG. 148 is a graph showing reduced CE-SDS analysis of AB1424/1612 F4 TriNKET after 6 freeze/thaw cycles compared to control.

FIG. 149 is a graph showing binding of AB1424/1612 F4 TriNKET to hBAFF-R+ cells after 6 freeze/thaw cycles (red) compared to control (blue).

FIG. 150 is a graph of a KHYG-1-CD16aV cytotoxicity assay of BJAB cells as induced by AB1424/1612 F4 TriNKET after 6 freeze/thaw cycles (red) and control (blue).

FIG. 151 is a chromatogram of SEC analysis of AB1424/1612 F4 TriNKET after agitation stress compared to control.

FIG. 152 is a graph showing reduced CE-SDS analysis of AB1424/1612 F4 TriNKET after agitation stress compared to control.

FIG. 153 is a graph showing binding of AB1424/1612 F4 TriNKET to hBAFF-R+ cells after agitation stress (red) compared to control (blue).

FIG. 154 is a graph of a KHYG-1-CD16aV cytotoxicity assay of BJAB cells as induced by AB1424/1612 F4 TriNKET after agitation stress (red) and control (blue).

FIG. 155A and FIG. 155B is a chromatogram of SEC analysis of AB1424/1612 F4 TriNKET Protein A eluate pre- (FIG. 155A) and post-low pH hold (FIG. 155B).

FIG. 156 is a graph showing cIEF profiling of AB1424/1612 F4 TriNKET after low pH hold compared to control.

FIG. 157 is a graph showing reduced CE-SDS analysis of AB1424/1612 F4 TriNKET after low pH hold compared to control.

FIG. 158 is a graph showing binding of AB1424/1612 F4 TriNKET to hBAFF-R+ cells after low pH hold (blue) compared to control (red).

FIG. 159 is a graph of a KHYG-1-CD16aV cytotoxicity assay of BJAB cells as induced by AB1424/1612 F4 TriNKET after low pH hold (green) and control (red).

FIG. 160A and FIG. 160B are graphs showing binding of AB1424/1612 F3′ TriNKET (blue), AB1424/1612 F4 TriNKET (red), and parental mAb (black) to KHYG-1 (FIG. 160A) and KHYG-1-CD16V (FIG. 160B) cell lines.

FIG. 161A and FIG. 161B are graphs showing percent surface retention of BAFF-R on RL cells exposed to AB1424/1612 F3′ TriNKET (blue), AB1424/1612 F4 TriNKET (red), and parental mAb (black) (FIG. 161A) and activated with IL-2 (FIG. 161B).

FIG. 162 is a graph showing percent surface retention of BAFF-R on Raji cells exposed to AB1424/1612 F3′ TriNKET (blue), AB1424/1612 F4 TriNKET (red), and parental mAb (black).

FIG. 163 is a graph of a resting human NK cell-induced cytotoxicity assay of RL cells following incubation with AB1424/1612 F3′ TriNKET (blue), AB1424/1612 F4 TriNKET (red), parental mAb (black), and human IgG1k (grey).

FIG. 164A and FIG. 164B are graphs of a rested human NK cell-induced cytotoxicity assay of RL cells following incubation with AB1424/1612 F3′ TriNKET (blue), AB1424/1612 F4 TriNKET (red), F3′ control (black), and F4 control (grey). Cells were co-cultured with control (FIG. 164A) or IL-2 (FIG. 164B).

FIG. 165 is a graph of a KHYG-1-CD16aV cytotoxicity assay of BJAB cells as induced by AB1424/1612 F3′ TriNKET (blue), AB1424/1612 F3′ TriNKET lacking NKG2D binding (black), or AB1424/1612 F3′ TriNKET-Fc silenced (red), or palivizumab F3′ TriNKET (grey).

FIG. 166 is a graph of a resting human NK cell-induced cytotoxicity assay of BJAB cells as induced by AB1424/1612 F3′ TriNKET (blue), AB1424/1612 F3′ TriNKET lacking NKG2D binding (black), or AB1424/1612 F3′ TriNKET-Fc silenced (red), or palivizumab F3′ TriNKET (grey).

FIG. 167 is a graph of a resting human NK cell-induced cytotoxicity assay of RL cells following incubation with AB1424/1612 F3′ TriNKET (blue), AB1424/1612 F4 TriNKET (red), AB1424/1612 F3′ TriNKET plus soluble MICA (black), and AB1424/1612 F4 TriNKET plus soluble MICA (grey).

FIG. 168 is a graph of a resting human NK cell-induced cytotoxicity assay of RL cells following incubation with AB1424/1612 F3′ TriNKET (blue), AB1424/1612 F4 TriNKET (red), AB1424/1612 F3′ TriNKET plus BAFF (black), and AB1424/1612 F4 TriNKET plus BAFF (grey).

FIG. 169 is a graph of interferon gamma (IFNγ) and CD107a production by BJAB cells following incubation with AB1424/1612 F3′ TriNKET (blue), AB1424/1612 F4 TriNKET (red), parental mAb (black), F3′-palivisumab (light grey), and F4-palivisumab (dark grey).

FIG. 170 is a graph of phagocytosis of BJAB cells by M0 macrophages following incubation with AB1424/1612 F3′ TriNKET (blue), AB1424/1612 F4 TriNKET (red), parental mAb (black), and Fc-silenced AB1424/1612 F3′ TriNKET (pink).

FIG. 171 is a graph of a human serum-induced cytotoxicity assay of Raji cells following incubation with rituximab (black), AB1424/1612 F3′ TriNKET (blue), or AB1424/1612 F3′ TriNKET.

FIG. 172A-FIG. 172E are histograms showing flow cytometry analysis of binding of AB1424/1612 F3′ TriNKET (blue) and F3′-palivizumab (red) to indicated BAFF-R+ cells in PBMCs.

FIG. 173A-FIG. 173F are histograms showing flow cytometry analysis of binding of AB1424/1612 F3′ TriNKET (blue) and F3′-palivizumab (red) to indicated cell types in human blood.

FIG. 174A-FIG. 174C are histograms showing flow cytometry analysis of binding of AB1424/1612 F3′ TriNKET (blue) and F3′-palivizumab (red) to human red blood cells.

FIG. 175A-FIG. 175F are graphs showing flow cytometry analysis of binding of (from left to right) AB1424/1612 F3′ TriNKET, F3′-palivizumab, AB1424/1612 F4 TriNKET, F4-palivizumab, and rituximab to indicated human donor PBMCs.

FIG. 176A-FIG. 176F are histograms showing flow cytometry analysis of binding of AB1424/1612 F3′ TriNKET (blue) and F3′-palivizumab (red) to indicated PBMCs from cynomolgus whole blood donor CYN317060.

FIG. 177A-FIG. 177F are graphs showing flow cytometry analysis of binding of (from left to right) AB1424/1612 F3′ TriNKET, F3′-palivizumab, AB1424/1612 F4 TriNKET, F4-palivizumab, and rituximab to indicated human donor PBMCs.

FIG. 178 is a graph showing CD107a positivity of CD16+CD8+NK cells in a co-culture of BJAB cells with PBMCs from cynomolgus whole blood donor CYN317060.

### DETAILED DESCRIPTION

The present application provides multispecific binding proteins that bind the NKG2D receptor and CD16 receptor on natural killer cells, and BAFF-R on a cancer cell or a B cell. In some embodiments, the multispecific proteins further include an additional antigen-binding site that binds BAFF-R. The application also provides pharmaceutical compositions comprising such multispecific binding proteins, and therapeutic methods using such multispecific proteins and pharmaceutical compositions, for purposes such as treating autoimmune diseases and cancer. Various aspects of the multispecific binding proteins described in the present application are set forth below in sections; however, aspects of the multispecific binding proteins described in one particular section are not to be limited to any particular section.

To facilitate an understanding of the present application, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The term “tumor-associated antigen” as used herein means any antigen including but not limited to a protein, glycoprotein, ganglioside, carbohydrate, or lipid that is associated with cancer. Such antigen can be expressed on malignant cells or in the tumor microenvironment such as on tumor-associated blood vessels, extracellular matrix, mesenchymal stroma, or immune infiltrates. In certain embodiments of the present disclosure, the terms “tumor-associated antigen” refers to BAFF-R, which is targeted by the second and/or the additional antigen-binding site present in a multispecific binding proteins of the present disclosure. It is understood, however, that BAFF-R may also be associated with diseases and disorders that are not tumor or cancer.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound described in the present application which, upon administration to a subject, is capable of providing a compound described in this application or an active metabolite or residue thereof. As is known to those of skill in the art, “salts” of the compounds described in the present application may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, though not in themselves pharmaceutically acceptable, may be employed in the preparation of salts useful as intermediates in obtaining the compounds described in the application and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds described in the present application compounded with a suitable cation such as Na⁺, NH₄⁺, and NW₄⁺ (wherein W is a C₁₋₄ alkyl group), and the like.

For therapeutic use, salts of the compounds described in the present application are contemplated as being pharmaceutically acceptable. However, salts of acids and bases that are non-pharmaceutically acceptable may also find use, for example, in the preparation or purification of a pharmaceutically acceptable compound.

As used herein, BAFF-R (also known as BAFF receptor, B-cell activating factor receptor, BR3, TNFRSF13C, tumor necrosis factor receptor superfamily member 13C, TNF receptor superfamily member 13C, CD268, and BLyS receptor 3) refers to the protein of Uniprot Accession No. Q96RJ3 and related isoforms and orthologs.

Throughout the description, where compositions are described as having, including, or comprising specific components, or where processes and methods are described as having, including, or comprising specific steps, it is contemplated that, additionally, there are compositions described in the present application that consist essentially of, or consist of, the recited components, and that there are processes and methods according to the present application that consist essentially of, or consist of, the recited processing steps.

As a general matter, compositions specifying a percentage are by weight unless otherwise specified. Further, if a variable is not accompanied by a definition, then the previous definition of the variable controls.

### I. Proteins

The present application provides multispecific binding proteins that bind to the NKG2D receptor and CD16 receptor on natural killer cells, and BAFF-R on a cancer cell. The multispecific binding proteins are useful in the pharmaceutical compositions and therapeutic methods described herein. Binding of the multispecific binding proteins to the NKG2D receptor and CD16 receptor on a natural killer cell enhances the activity of the natural killer cell toward destruction of tumor cells expressing BAFF-R antigen. Binding of the multispecific binding proteins to BAFF-R-expressing cells brings the cancer cells into proximity with the natural killer cell, which facilitates direct and indirect destruction of the tumor cells by the natural killer cell. Multispecific binding proteins that bind NKG2D, CD16, and another target are disclosed in International Application Publication Nos. WO2018148445 and WO2019157366, which are not incorporated herein by reference. Further description of some exemplary multispecific binding proteins is provided below.

The first component of the multispecific binding protein is an antigen-binding site that binds to NKG2D receptor-expressing cells, which can include but are not limited to NK cells, γδ T cells and CD8⁺ αβ T cells. Upon NKG2D binding, the multispecific binding proteins may block natural ligands, such as ULBP6 and MICA, from binding to NKG2D and activating NK cells.

The second component of the multispecific binding protein is an antigen-binding site that binds to BAFF-R. The BAFF-R-expressing cells may be found, for example, in B-cell non-Hodgkin's lymphoma (B-NHL), such as chronic lymphocytic leukemia (CLL), mantle cell lymphoma (MCL), follicular lymphoma (FL), diffuse large B-cell lymphoma (DLBCL), marginal zone lymphoma, mucosa-associated lymphoid tissue (MALT) lymphoma, primary mediastinal B-cell lymphoma, acute lymphocytic leukemia (ALL); and autoimmune inflammatory diseases.

The third component of the multispecific binding proteins is an antibody Fc domain or a portion thereof, or an antigen-binding site that binds to cells expressing CD16, an Fc receptor on the surface of leukocytes including natural killer cells, macrophages, neutrophils, eosinophils, mast cells, and follicular dendritic cells.

An additional antigen-binding site of the multispecific binding proteins may bind BAFF-R. In certain embodiments, the first antigen-binding site that binds NKG2D is an scFv, and the second and the additional antigen-binding sites that bind BAFF-R are each a Fab fragment. In certain embodiments, the first antigen-binding site that binds NKG2D is an scFv, and the second and the additional antigen-binding sites that bind BAFF-R are each an scFv. In certain embodiments, the first antigen-binding site that binds NKG2D is a Fab fragment, and the second and the additional antigen-binding sites that bind BAFF-R are each an scFv. In certain embodiments, the first antigen-binding site that binds NKG2D is a Fab, and the second and the additional antigen-binding sites that bind BAFF-R are each a Fab fragment.

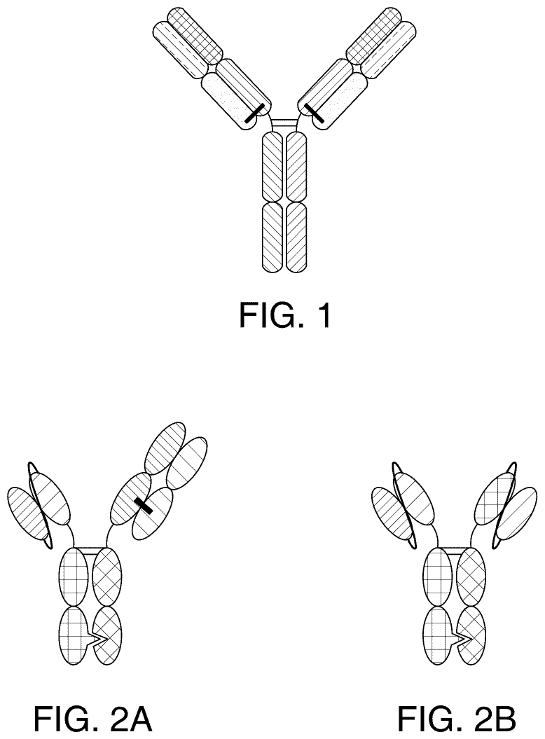

The multispecific binding proteins described herein can take various formats. For example, one format is a heterodimeric, multispecific antibody including a first immunoglobulin heavy chain, a first immunoglobulin light chain, a second immunoglobulin heavy chain and a second immunoglobulin light chain (FIG. 1). The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and optionally a first light chain constant domain. The first immunoglobulin light chain, together with the first immunoglobulin heavy chain, forms an antigen-binding site that binds NKG2D. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and optionally a second light chain constant domain. The second immunoglobulin light chain, together with the second immunoglobulin heavy chain, forms an antigen-binding site that binds BAFF-R. In some embodiments, the first Fc domain and second Fc domain together are able to bind to CD16 (FIG. 1). In some embodiments, the first immunoglobulin light chain is identical to the second immunoglobulin light chain.

The antigen-binding sites may each incorporate an antibody heavy chain variable domain and an antibody light chain variable domain (e.g., arranged as in an antibody, or fused together to form an scFv), or one or more of the antigen-binding sites may be a single domain antibody, such as a V_{H}H antibody like a camelid antibody or a V_{NAR} antibody like those found in cartilaginous fish.

In some embodiments, the second antigen-binding site incorporates a light chain variable domain having an amino acid sequence identical to the amino acid sequence of the light chain variable domain present in the first antigen-binding site.

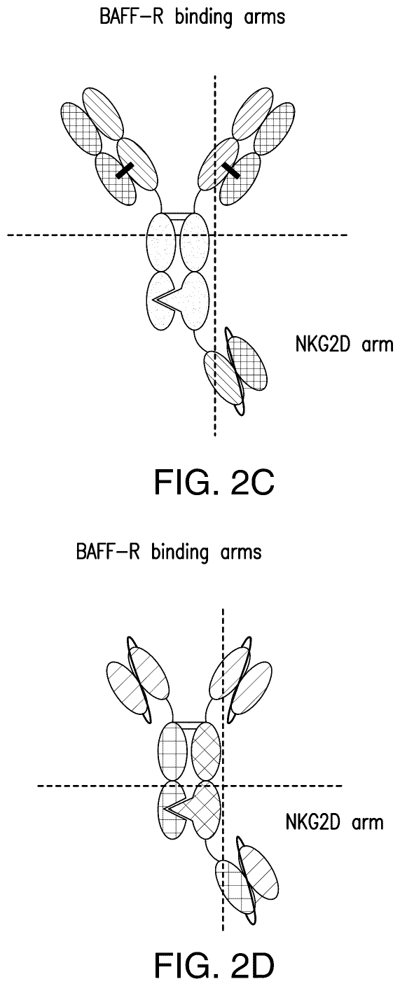

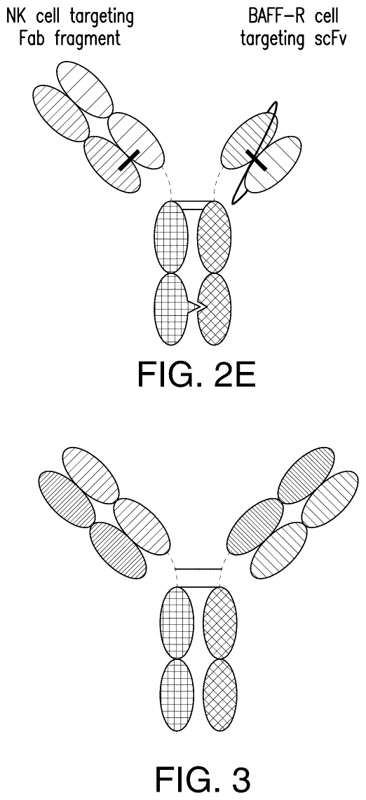

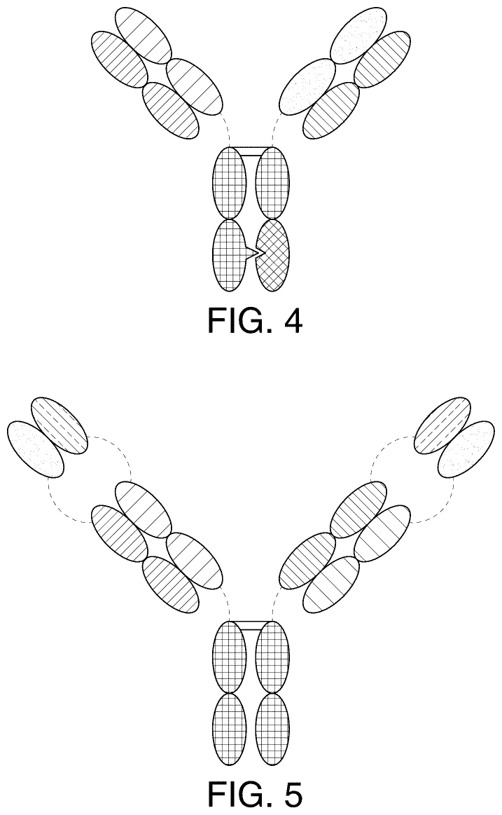

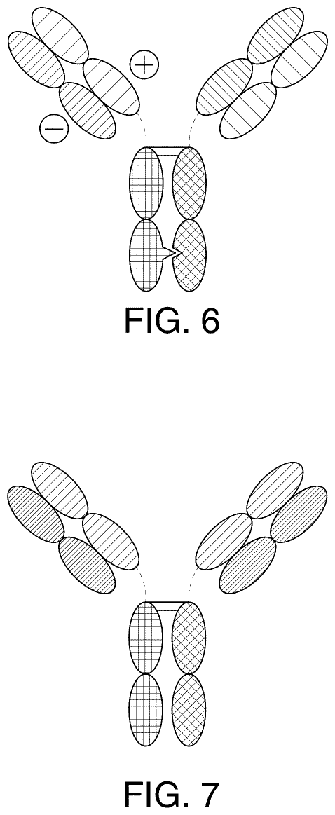

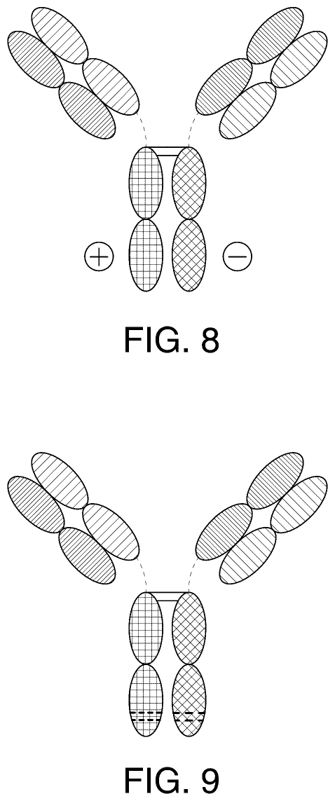

Another exemplary format involves a heterodimeric, multispecific antibody including a first immunoglobulin heavy chain, a second immunoglobulin heavy chain and an immunoglobulin light chain (e.g., FIG. 2A). In some embodiments, the first immunoglobulin heavy chain includes a first Fc (hinge-CH2-CH3) domain fused via either a linker or an antibody hinge to a single-chain variable fragment (scFv) composed of a heavy chain variable domain and light chain variable domain which pair and bind NKG2D, or bind BAFF-R. In some embodiments, the second immunoglobulin heavy chain includes a second Fc (hinge-CH2-CH3) domain, a second heavy chain variable domain and a CH1 heavy chain domain. The immunoglobulin light chain includes a light chain variable domain and a light chain constant domain. In some embodiments, the second immunoglobulin heavy chain pairs with the immunoglobulin light chain and binds to NKG2D or binds BAFF-R with the proviso that when the first Fc domain is fused to an scFv that binds NKG2D, the second immunoglobulin heavy chain paired with the immunoglobulin light chain binds BAFF-R but not NKG2D, and vice versa. In some embodiments, the scFv in the first immunoglobulin heavy chain binds BAFF-R; and the heavy chain variable domain in the second immunoglobulin heavy chain and the light chain variable domain in the immunoglobulin light chain, when paired, bind NKG2D (e.g., FIG. 2E). In some embodiments, the scFv in the first immunoglobulin heavy chain binds NKG2D; and the heavy chain variable domain in the second immunoglobulin heavy chain and the light chain variable domain in the immunoglobulin light chain, when paired, bind BAFF-R. In some embodiments, the first Fc domain and the second Fc domain together are able to bind to CD16 (e.g., FIG. 2A). In some embodiments, the first Fc domain and the second Fc domain together are able to bind to CD16 (e.g., FIG. 2A).

Another exemplary format involves a heterodimeric, multispecific antibody including a first immunoglobulin heavy chain, and a second immunoglobulin heavy chain (e.g., FIG. 2B). In some embodiments, the first immunoglobulin heavy chain includes a first Fc (hinge-CH2-CH3) domain fused via either a linker or an antibody hinge to a single-chain variable fragment (scFv) composed of a heavy chain variable domain and light chain variable domain, which pair and bind NKG2D, or bind BAFF-R. In some embodiments, the second immunoglobulin heavy chain includes a second Fc (hinge-CH2-CH3) domain fused via either a linker or an antibody hinge to a single-chain variable fragment (scFv) composed of a heavy chain variable domain and light chain variable domain which pair and bind NKG2D or bind BAFF-R, with the proviso that when the first Fc domain is fused to an scFv that binds NKG2D, the second Fc domain fused to an scFv binds BAFF-R, but not NKG2D, and vice versa. In some embodiments, the first Fc domain and the second Fc domain together are able to bind to CD16 (e.g., FIG. 2B).

In some embodiments, the single-chain variable fragment (scFv) described above is linked to the antibody constant domain via a hinge sequence. In some embodiments, the hinge comprises amino acids Ala-Ser or Gly-Ser. In some embodiments, the hinge comprises amino acids Ala-Ser or Gly-Ser. In some embodiments, the hinge connecting an scFv (e.g., an scFv that binds NKG2D or an scFv that binds BAFF-R) and the antibody heavy chain constant domain comprises amino acids Ala-Ser. In some embodiments, the hinge connecting an scFv (e.g., an scFv that binds NKG2D or an scFv that binds BAFF-R) and the antibody heavy chain constant domain comprises amino acids Gly-Ser. In some other embodiments, the hinge comprises amino acids Ala-Ser and Thr-Lys-Gly. The hinge sequence can provide flexibility of binding to the target antigen, and balance between flexibility and optimal geometry.

In some embodiments, the single-chain variable fragment (scFv) described above includes a heavy chain variable domain and a light chain variable domain. In some embodiments, the heavy chain variable domain forms a disulfide bridge with the light chain variable domain to enhance stability of the scFv. For example, a disulfide bridge can be formed between the C44 residue of the heavy chain variable domain and the C100 residue of the light chain variable domain, the amino acid positions numbered under Kabat. In some embodiments, the heavy chain variable domain is linked to the light chain variable domain via a flexible linker. Any suitable linker can be used, for example, the (G₄S)₄ linker ((GlyGlyGlyGlySer)₄ (SEQ ID NO:119)). In some embodiments of the scFv, the heavy chain variable domain is positioned at the N-terminus of the light chain variable domain. In some embodiments of the scFv, the heavy chain variable domain is positioned at the C terminus of the light chain variable domain.

The multispecific binding proteins described herein can further include one or more additional antigen-binding sites. The additional antigen-binding site(s) may be fused to the N-terminus of the constant region CH2 domain or to the C-terminus of the constant region CH3 domain, optionally via a linker sequence. In certain embodiments, the additional antigen-binding site(s) takes the form of a single-chain variable region (scFv) that is optionally disulfide-stabilized, resulting in a tetravalent or trivalent multispecific binding protein. For example, a multispecific binding protein includes a first antigen-binding site that binds NKG2D, a second antigen-binding site that binds BAFF-R, an additional antigen-binding site that binds BAFF-R, and an antibody constant region or a portion thereof sufficient to bind CD16 or a fourth antigen-binding site that binds CD16. Any one of these antigen binding sites can either take the form of a Fab fragment or an scFv, such as an scFv described above.

In some embodiments, the additional antigen-binding site binds a different epitope of BAFF-R from the second antigen-binding site. In some embodiments, the additional antigen-binding site binds the same epitope as the second antigen-binding site. In some embodiments, the additional antigen-binding site comprises the same heavy chain and light chain CDR sequences as the second antigen-binding site. In some embodiments, the additional antigen-binding site comprises the same heavy chain and light chain variable domain sequences as the second antigen-binding site. In some embodiments, the additional antigen-binding site has the same amino acid sequence(s) as the second antigen-binding site. In some embodiments, the additional antigen-binding site comprises heavy chain and light chain variable domain sequences that are different from the heavy chain and light chain variable domain sequences of the second antigen-binding site. In some embodiments, the additional antigen-binding site has an amino acid sequence that is different from the sequence of the second antigen-binding site. In some embodiments, the second antigen-binding site and the additional antigen-binding site bind different tumor-associated antigens. In some embodiments, the second antigen-binding site and the additional antigen-binding site binds different antigens. Exemplary formats are shown in FIG. 2C and FIG. 2D. Accordingly, the multispecific binding proteins can provide bivalent engagement of BAFF-R. Bivalent engagement of BAFF-R by the multispecific proteins can stabilize BAFF-R on the tumor cell surface and enhance cytotoxicity of NK cells towards the tumor cells. Bivalent engagement of BAFF-R by the multispecific proteins can confer stronger binding of the multispecific proteins to the tumor cells, thereby facilitating stronger cytotoxic response of NK cells towards the tumor cells, especially towards tumor cells expressing a low level of BAFF-R.

The multispecific binding proteins can take additional formats. In some embodiments, the multispecific binding protein is in the Triomab form, which is a trifunctional, bispecific antibody that maintains an IgG-like shape. This chimera consists of two half antibodies, each with one light and one heavy chain, that originate from two parental antibodies.

In some embodiments, the multispecific binding protein is in a KiH Common Light Chain (LC) form, which incorporates the knobs-into-holes (KiH) technology (e.g., the multispecific binding protein represented in FIG. 21). The KiH Common LC form is a heterodimer comprising a Fab which binds to a first target, a Fab which binds to a second target, and an Fc domain stabilized by heterodimerization mutations. The two Fabs each comprise a heavy chain and light chain, wherein the heavy chain of each Fab differs from the other, and the light chain that pairs with each respective heavy chain is common to both Fabs.

In some embodiments, the multispecific binding protein is the KiH form, which involves the knobs-into-holes (KiHs) technology. The KiH involves engineering CH3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structured-guided phage library screening (Atwell S, Ridgway J B, Wells J A, Carter P., Stable heterodimers from remodeling the domain interface of a homodimer using a phage display library, J. Mol. Biol. (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc variant with improved affinity for FcγRs. Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the multispecific binding protein is in the dual-variable domain immunoglobulin (DVD-Ig™) form, which combines the target binding domains of two monoclonal antibodies via flexible naturally occurring linkers, and yields a tetravalent IgG-like molecule.

In some embodiments, the multispecific binding protein is in the Orthogonal Fab interface (Ortho-Fab) form. In the ortho-Fab IgG approach (Lewis S M, Wu X, Pustilnik A, Sereno A, Huang F, Rick H L, et al., Generation of bispecific IgG antibodies by structure-based design of an orthogonal Fab interface. Nat. Biotechnol. (2014) 32(2):191-8), structure-based regional design introduces complementary mutations at the LC and HC_{VH-CH1} interface in only one Fab fragment, without any changes being made to the other Fab fragment.

In some embodiments, the multispecific binding protein is in the 2-in-1 Ig format. In some embodiments, the multispecific binding protein is in the ES form, which is a heterodimeric construct containing two different Fab fragments binding to targets 1 and target 2 fused to the Fc. Heterodimerization is ensured by electrostatic steering mutations in the Fc.

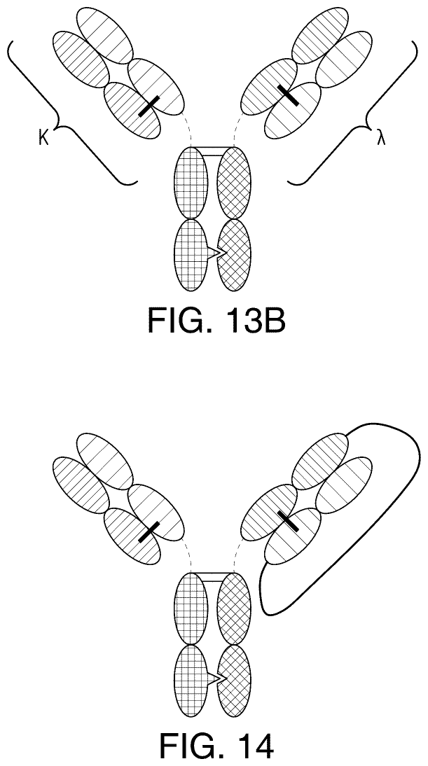

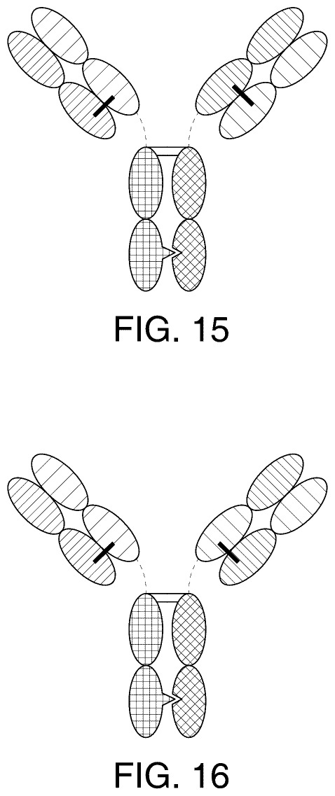

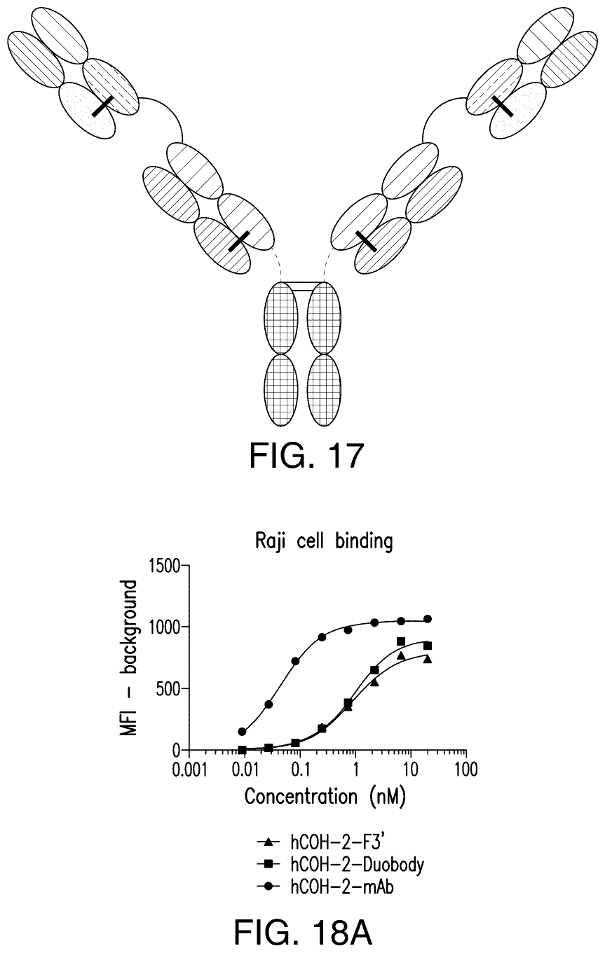

In some embodiments, the multispecific binding protein is in the κλ-Body form, which is a heterodimeric construct with two different Fab fragments fused to Fc stabilized by heterodimerization mutations: Fab fragment 1 targeting antigen 1 contains kappa LC, and Fab fragment 2 targeting antigen 2 contains lambda LC. FIG. 13A is an exemplary representation of one form of a κλ-Body; FIG. 13B is an exemplary representation of another κλ-Body.

In some embodiments, the multispecific binding protein is in Fab Arm Exchange form (antibodies that exchange Fab fragment arms by swapping a heavy chain and attached light chain (half-molecule) with a heavy-light chain pair from another molecule, which results in bispecific antibodies).

In some embodiments, the multispecific binding protein is in the SEED Body form. The strand-exchange engineered domain (SEED) platform was designed to generate asymmetric and bispecific antibody-like molecules, a capability that expands therapeutic applications of natural antibodies. This protein engineering platform is based on exchanging structurally related sequences of immunoglobulin within the conserved CH3 domains. The SEED design allows efficient generation of AG/GA heterodimers, whereas disfavoring homodimerization of AG and GA SEED CH3 domains. (Muda M. et al., Protein Eng. Des. Sel. (2011, 24(5):447-54)).

In some embodiments, the multispecific binding protein is in the LuZ-Y form, in which a leucine zipper is used to induce heterodimerization of two different HCs. (Wranik, B J. et al., J. Biol. Chem. (2012), 287:43331-9).

In some embodiments, the multispecific binding protein is in the Cov-X-Body form. In bispecific CovX-Bodies, two different peptides are joined together using a branched azetidinone linker and fused to the scaffold antibody under mild conditions in a site-specific manner. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V R et al., PNAS (2010), 107(52); 22611-22616).

In some embodiments, the multispecific binding protein is in an OAsc-Fab heterodimeric form that includes Fab fragment binding to target 1, and scFab binding to target 2 fused to Fc. Heterodimerization is ensured by mutations in the Fc.

In some embodiments, the multispecific binding protein is in a DuetMab form, which is a heterodimeric construct containing two different Fab fragments binding to antigens 1 and 2, and Fc stabilized by heterodimerization mutations. Fab fragments 1 and 2 contain differential S-S bridges that ensure correct LC and HC pairing.

In some embodiments, the multispecific binding protein is in a CrossmAb form, which is a heterodimeric construct with two different Fab fragments binding to targets 1 and 2, fused to Fc stabilized by heterodimerization. CL and CH1 domains and VH and VL domains are switched, e.g., CH1 is fused in-frame with VL, and CL is fused in-frame with VH.

In some embodiments, the multispecific binding protein is in a Fit-Ig form, which is a homodimeric construct where Fab fragment binding to antigen 2 is fused to the N terminus of HC of Fab fragment that binds to antigen 1. The construct contains wild-type Fc.

Individual components of the multispecific binding proteins are described in more detail below.

### NKG2D-Binding Site

Upon binding to the NKG2D receptor and CD16 receptor on natural killer cells, and BAFF-R, the multispecific binding proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys. In some embodiments, the proteins can agonize NK cells in humans and in other species such as cynomolgus monkeys.

Table 1 lists peptide sequences of heavy chain variable domains and light chain variable domains that, in combination, can bind to NKG2D. In some embodiments, the heavy chain variable domain and the light chain variable domain are arranged in Fab format. In some embodiments, the heavy chain variable domain and the light chain variable domain are fused together to form an scFv.

The NKG2D binding sites listed in Table 1 can vary in their binding affinity to NKG2D, nevertheless, they all activate human NK cells.

Unless indicated otherwise, the CDR sequences provided in Table 1 are determined under Kabat numbering.

TABLE 1

| | Heavy chain variable region amino | Light chain variable region amino |
|---|---|---|
| Clones | acid sequence | acid sequence |
| ADI- | QVQLQQWGAGLLKPSETLSLTCAV | DIQMTQSPSTLSASVGDRVTITCR |
| 27705 | YGGSFSGYYWSWIRQPPGKGLEWI | ASQSISSWLAWYQQKPGKAPKLLI |
| | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYNSYPITF |
| | RGPWSFDPWGQGTLVTVSS | GGGTKVEIK |
| | (SEQ ID NO: 1) | (SEQ ID NO: 5) |
| | CDR1 (SEQ ID NO: 2)-GSFSGYYWS | |
| | CDR2 (SEQ ID NO: 3)- | |
| | EIDHSGSTNYNPSLKS | |
| | CDR3 (SEQ ID NO: 4)- | |
| | ARARGPWSFDP | |
| ADI- | QVQLQQWGAGLLKPSETLSLTCAV | EIVLTQSPGTLSLSPGERATLSCRA |
| 27724 | YGGSFSGYYWSWIRQPPGKGLEWI | SQSVSSSYLAWYQQKPGQAPRLLI |
| | GEIDHSGSTNYNPSLKSRVTISVDTS | YGASSRATGIPDRFSGSGSGTDFTL |
| | KNQFSLKLSSVTAADTAVYYCARA | TISRLEPEDFAVYYCQQYGSSPITF |
| | RGPWSFDPWGQGTLVTVSS | GGGTKVEIK |
| | (SEQ ID NO: 1) | (SEQ ID NO: 6) |
| ADI- | QVQLQQWGAGLLKPSETLSLTCAV | DIQMTQSPSTLSASVGDRVTITCR |
| 27740 | YGGSFSGYYWSWIRQPPGKGLEWI | ASQSIGSWLAWYQQKPGKAPKLL |
| (A40) | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYHSFYT |
| | RGPWSFDPWGQGTLVTVSS | FGGGTKVEIK |
| | (SEQ ID NO: 1) | (SEQ ID NO: 7) |
| ADI- | QVQLQQWGAGLLKPSETLSLTCAV | DIQMTQSPSTLSASVGDRVTITCR |
| 27741 | YGGSFSGYYWSWIRQPPGKGLEWI | ASQSIGSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQSNSYYT |
| | RGPWSFDPWGQGTLVTVSS | FGGGTKVEIK |
| | (SEQ ID NO: 1) | (SEQ ID NO: 8) |
| ADI- | QVQLQQWGAGLLKPSETLSLTCAV | DIQMTQSPSTLSASVGDRVTITCR |
| 27743 | YGGSFSGYYWSWIRQPPGKGLEWI | ASQSISSWLAWYQQKPGKAPKLLI |
| | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYNSYPTF |
| | RGPWSFDPWGQGTLVTVSS | GGGTKVEIK |
| | (SEQ ID NO: 1) | (SEQ ID NO: 9) |
| ADI- | QVQLQQWGAGLLKPSETLSLTCAV | ELQMTQSPSSLSASVGDRVTITCR |
| 28153 | YGGSFSGYYWSWIRQPPGKGLEWI | TSQSISSYLNWYQQKPGQPPKLLI |
| | GEIDHSGSTNYNPSLKSRVTISVDTS | YWASTRESGVPDRFSGSGSGTDFT |
| | KNQFSLKLSSVTAADTAVYYCARA | LTISSLQPEDSATYYCQQSYDIPYT |
| | RGPWGFDPWGQGTLVTVSS | FGQGTKLEIK |
| | (SEQ ID NO: 10) | (SEQ ID NO: 11) |
| ADI- | QVQLQQWGAGLLKPSETLSLTCAV | DIQMTQSPSTLSASVGDRVTITCR |
| 28226 | YGGSFSGYYWSWIRQPPGKGLEWI | ASQSISSWLAWYQQKPGKAPKLLI |
| (C26) | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYGSFPITF |
| | RGPWSFDPWGQGTLVTVSS | GGGTKVEIK |
| | (SEQ ID NO: 1) | (SEQ ID NO: 12) |
| ADI- | QVQLQQWGAGLLKPSETLSLTCAV | DIQMTQSPSTLSASVGDRVTITCR |
| 28154 | YGGSFSGYYWSWIRQPPGKGLEWI | ASQSISSWLAWYQQKPGKAPKLLI |
| | GEIDHSGSTNYNPSLKSRVTISVDTS | YKASSLESGVPSRFSGSGSGTDFTL |
| | KNQFSLKLSSVTAADTAVYYCARA | TISSLQPDDFATYYCQQSKEVPWT |
| | RGPWSFDPWGQGTLVTVSS | FGQGTKVEIK |
| | (SEQ ID NO: 1) | (SEQ ID NO: 13) |
| ADI- | QVQLQQWGAGLLKPSETLSLTCAV | DIQMTQSPSTLSASVGDRVTITCR |
| 29399 | YGGSFSGYYWSWIRQPPGKGLEWI | ASQSISSWLAWYQQKPGKAPKLLI |
| | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYNSFPTF |
| | RGPWSFDPWGQGTLVTVSS | GGGTKVEIK |
| | (SEQ ID NO: 1) | (SEQ ID NO: 14) |
| ADI- | QVQLQQWGAGLLKPSETLSLTCAV | DIQMTQSPSTLSASVGDRVTITCR |
| 29401 | YGGSFSGYYWSWIRQPPGKGLEWI | ASQSIGSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYDIYPTF |
| | RGPWSFDPWGQGTLVTVSS | GGGTKVEIK |
| | (SEQ ID NO: 1) | (SEQ ID NO: 15) |
| ADI- | QVQLQQWGAGLLKPSETLSLTCAV | DIQMTQSPSTLSASVGDRVTITCR |
| 29403 | YGGSFSGYYWSWIRQPPGKGLEWI | ASQSISSWLAWYQQKPGKAPKLLI |
| | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYDSYPTF |
| | RGPWSFDPWGQGTLVTVSS | GGGTKVEIK |
| | (SEQ ID NO: 1) | (SEQ ID NO: 16) |
| ADI- | QVQLQQWGAGLLKPSETLSLTCAV | DIQMTQSPSTLSASVGDRVTITCR |
| 29405 | YGGSFSGYYWSWIRQPPGKGLEWI | ASQSISSWLAWYQQKPGKAPKLLI |
| | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYGSFPTF |
| | RGPWSFDPWGQGTLVTVSS | GGGTKVEIK |
| | (SEQ ID NO: 1) | (SEQ ID NO: 17) |
| ADI- | QVQLQQWGAGLLKPSETLSLTCAV | DIQMTQSPSTLSASVGDRVTITCR |
| 29407 | YGGSFSGYYWSWIRQPPGKGLEWI | ASQSISSWLAWYQQKPGKAPKLLI |
| | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYQSFPTF |
| | RGPWSFDPWGQGTLVTVSS | GGGTKVEIK |
| | (SEQ ID NO: 1) | (SEQ ID NO: 18) |
| ADI- | QVQLQQWGAGLLKPSETLSLTCAV | DIQMTQSPSTLSASVGDRVTITCR |
| 29419 | YGGSFSGYYWSWIRQPPGKGLEWI | ASQSISSWLAWYQQKPGKAPKLLI |
| | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYSSFSTFG |
| | RGPWSFDPWGQGTLVTVSS | GGTKVEIK |
| | (SEQ ID NO: 1) | (SEQ ID NO: 19) |
| ADI- | QVQLQQWGAGLLKPSETLSLTCAV | DIQMTQSPSTLSASVGDRVTITCR |
| 29421 | YGGSFSGYYWSWIRQPPGKGLEWI | ASQSISSWLAWYQQKPGKAPKLLI |
| | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYESYSTF |
| | RGPWSFDPWGQGTLVTVSS | GGGTKVEIK |
| | (SEQ ID NO: 1) | (SEQ ID NO: 20) |
| ADI- | QVQLQQWGAGLLKPSETLSLTCAV | DIQMTQSPSTLSASVGDRVTITCR |
| 29424 | YGGSFSGYYWSWIRQPPGKGLEWI | ASQSISSWLAWYQQKPGKAPKLLI |
| | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYDSFITFG |
| | RGPWSFDPWGQGTLVTVSS | GGTKVEIK |
| | (SEQ ID NO: 1) | (SEQ ID NO: 21) |
| ADI- | QVQLQQWGAGLLKPSETLSLTCAV | DIQMTQSPSTLSASVGDRVTITCR |
| 29425 | YGGSFSGYYWSWIRQPPGKGLEWI | ASQSISSWLAWYQQKPGKAPKLLI |
| | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYQSYPTF |
| | RGPWSFDPWGQGTLVTVSS | GGGTKVEIK |
| | (SEQ ID NO: 1) | (SEQ ID NO: 22) |
| ADI- | QVQLQQWGAGLLKPSETLSLTCAV | DIQMTQSPSTLSASVGDRVTITCR |
| 29426 | YGGSFSGYYWSWIRQPPGKGLEWI | ASQSIGSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYHSFPTF |
| | RGPWSFDPWGQGTLVTVSS | GGGTKVEIK |
| | (SEQ ID NO: 1) | (SEQ ID NO: 23) |
| ADI- | QVQLQQWGAGLLKPSETLSLTCAV | DIQMTQSPSTLSASVGDRVTITCR |
| 29429 | YGGSFSGYYWSWIRQPPGKGLEWI | ASQSIGSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYELYSY |
| | RGPWSFDPWGQGTLVTVSS | TFGGGTKVEIK |
| | (SEQ ID NO: 1) | (SEQ ID NO: 24) |
| ADI- | QVQLQQWGAGLLKPSETLSLTCAV | DIQMTQSPSTLSASVGDRVTITCR |
| 29447 | YGGSFSGYYWSWIRQPPGKGLEWI | ASQSISSWLAWYQQKPGKAPKLLI |
| (F47) | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCQQYDTFITFG |
| | RGPWSFDPWGQGTLVTVSS | GGTKVEIK |
| | (SEQ ID NO: 1) | (SEQ ID NO: 25) |
| ADI- | QVQLVQSGAEVKKPGSSVKVSCKA | DIVMTQSPDSLAVSLGERATINCK |
| 27727 | SGGTFSSYAISWVRQAPGQGLEWM | SSQSVLYSSNNKNYLAWYQQKPG |
| | GGIIPIFGTANYAQKFQGRVTITADE | QPPKLLIYWASTRESGVPDRFSGS |
| | STSTAYMELSSLRSEDTAVYYCAR | GSGTDFTLTISSLQAEDVAVYYCQ |
| | GDSSIRHAYYYYGMDVWGQGTTV | QYYSTPITFGGGTKVEIK |
| | TVSS | (SEQ ID NO: 32) |
| | (SEQ ID NO: 26) | CDR1 (SEQ ID NO: 33)- |
| | CDR1-GTFSSYAIS (non-Kabat) (SEQ | KSSQSVLYSSNNKNYLA |
| | ID NO: 27) or SYAIS (SEQ ID NO: 28) | CDR2 (SEQ ID NO: 34)- |
| | CDR2 (SEQ ID NO: 29)- | WASTRES |
| | GIIPIFGTANYAQKFQG | CDR3 (SEQ ID NO: 35)- |
| | CDR3-ARGDSSIRHAYYYYGMDV | QQYYSTPIT |
| | (non-Kabat) (SEQ ID NO: 30) or | |
| | GDSSIRHAYYYYGMDV (SEQ ID | |
| | NO: 31) | |
| ADI- | QLQLQESGPGLVKPSETLSLTCTVS | EIVLTQSPATLSLSPGERATLSCRA |
| 29443 | GGSISSSSYYWGWIRQPPGKGLEWI | SQSVSRYLAWYQQKPGQAPRLLI |
| (F43) | GSIYYSGSTYYNPSLKSRVTISVDTS | YDASNRATGIPARFSGSGSGTDFT |
| | KNQFSLKLSSVTAADTAVYYCARG | LTISSLEPEDFAVYYCQQFDTWPP |
| | SDRFHPYFDYWGQGTLVTVSS | TFGGGTKVEIK |
| | (SEQ ID NO: 36) | (SEQ ID NO: 42) |
| | CDR1-GSISSSSYYWG (non-Kabat) | CDR1 (SEQ ID NO: 43)- |
| | (SEQ ID NO: 37) or SSSYYWG (SEQ | RASQSVSRYLA |
| | ID NO: 38) | CDR2 (SEQ ID NO: 44)- |
| | CDR2 (SEQ ID NO: 39)- | DASNRAT |
| | SIYYSGSTYYNPSLKS | CDR3 (SEQ ID NO: 45)- |
| | CDR3-ARGSDRFHPYFDY (non- | QQFDTWPPT |
| | Kabat) (SEQ ID NO: 40) or | |
| | GSDRFHPYFDY (SEQ ID NO: 41) | |
| ADI- | QVQLQQWGAGLLKPSETLSLTCAV | DIQMTQSPSTLSASVGDRVTITCR |
| 29404 | YGGSFSGYYWSWIRQPPGKGLEWI | ASQSISSWLAWYQQKPGKAPKLLI |
| (F04) | GEIDHSGSTNYNPSLKSRVTISVDTS | YKASSLESGVPSRFSGSGSGTEFTL |
| | KNQFSLKLSSVTAADTAVYYCARA | TISSLQPDDFATYYCEQYDSYPTF |
| | RGPWSFDPWGQGTLVTVSS (SEQ | GGGTKVEIK |
| | ID NO: 1) | (SEQ ID NO: 46) |
| ADI- | QVQLVQSGAEVKKPGSSVKVSCKA | DIVMTQSPDSLAVSLGERATINCE |
| 28200 | SGGTFSSYAISWVRQAPGQGLEWM | SSQSLLNSGNQKNYLTWYQQKPG |
| | GGIIPIFGTANYAQKFQGRVTITADE | QPPKPLIYWASTRESGVPDRFSGS |
| | STSTAYMELSSLRSEDTAVYYCARR | GSGTDFTLTISSLQAEDVAVYYCQ |
| | GRKASGSFYYYYGMDVWGQGTTV | NDYSYPYTFGQGTKLEIK |
| | TVSS | (SEQ ID NO: 49) |
| | (SEQ ID NO: 47) | CDR1 (SEQ ID NO: 50)- |
| | CDR1 (SEQ ID NO: 27)-GTFSSYAIS | ESSQSLLNSGNQKNYLT |
| | CDR2 (SEQ ID NO: 29)- | CDR2 (SEQ ID NO: 34)-WASTRES |
| | GIIPIFGTANYAQKFQG | CDR3 (SEQ ID NO: 51)- |
| | CDR3 (SEQ ID NO: 48)- | QNDYSYPYT |
| | ARRGRKASGSFYYYYGMDV | |
| ADI- | QVQLVQSGAEVKKPGASVKVSCKA | EIVMTQSPATLSVSPGERATLSCR |
| 29379 | SGYTFTSYYMHWVRQAPGQGLEW | ASQSVSSNLAWYQQKPGQAPRLLI |
| (E79) | MGIINPSGGSTSYAQKFQGRVTMTR | YGASTRATGIPARFSGSGSGTEFTL |
| | DTSTSTVYMELSSLRSEDTAVYYCA | TISSLQSEDFAVYYCQQYDDWPFT |
| | RGAPNYGDTTHDYYYMDVWGKG | FGGGTKVEIK |
| | TTVTVSS | (SEQ ID NO: 58) |
| | (SEQ ID NO: 52) | CDR1 (SEQ ID NO: 59)- |
| | CDR1 (SEQ ID NO: 53)- | RASQSVSSNLA |
| | YTFTSYYMH (non-Kabat) or SYYMH | CDR2 (SEQ ID NO: 60)-GASTRAT |
| | (SEQ ID NO: 54) | CDR3 (SEQ ID NO: 61)- |
| | CDR2 (SEQ ID NO: 55)- | QQYDDWPFT |
| | IINPSGGSTSYAQKFQG | |
| | CDR3- | |
| | ARGAPNYGDTTHDYYYMDV (non- | |
| | Kabat) (SEQ ID NO: 56) or | |
| | GAPNYGDTTHDYYYMDV (SEQ ID | |
| | NO: 57) | |
| ADI- | QVQLVQSGAEVKKPGASVKVSCKA | EIVLTQSPGTLSLSPGERATLSCRA |
| 29463 | SGYTFTGYYMHWVRQAPGQGLEW | SQSVSSNLAWYQQKPGQAPRLLIY |
| (F63) | MGWINPNSGGTNYAQKFQGRVTM | GASTRATGIPARFSGSGSGTEFTLT |
| | TRDTSISTAYMELSRLRSDDTAVYY | ISSLQSEDFAVYYCQQDDYWPPTF |
| | CARDTGEYYDTDDHGMDVWGQG | GGGTKVEIK |
| | TTVTVSS | (SEQ ID NO: 68) |
| | (SEQ ID NO: 62) | CDR1 (SEQ ID NO: 59)- |
| | CDR1-YTFTGYYMH (non-Kabat) | RASQSVSSNLA |
| | (SEQ ID NO: 63) or GYYMH (SEQ ID | CDR2 (SEQ ID NO: 60)-GASTRAT |
| | NO: 64) | CDR3 (SEQ ID NO: 69)- |
| | CDR2 (SEQ ID NO: 65)- | QQDDYWPPT |
| | WINPNSGGTNYAQKFQG | |
| | CDR3-ARDTGEYYDTDDHGMDV | |
| | (non-Kabat) (SEQ ID NO: 66) or | |
| | DTGEYYDTDDHGMDV (SEQ ID | |
| | NO: 67) | |
| ADI- | EVQLLESGGGLVQPGGSLRLSCAAS | DIQMTQSPSSVSASVGDRVTITCR |
| 27744 | GFTFSSYAMSWVRQAPGKGLEWVS | ASQGIDSWLAWYQQKPGKAPKLL |
| (A44) | AISGSGGSTYYADSVKGRFTISRDN | IYAASSLQSGVPSRFSGSGSGTDFT |
| | SKNTLYLQMNSLRAEDTAVYYCAK | LTISSLQPEDFATYYCQQGVSYPR |
| | DGGYYDSGAGDYWGQGTLVTVSS | TFGGGTKVEIK |
| | (SEQ ID NO: 70) | (SEQ ID NO: 75) |
| | CDR1-FTFSSYAMS (non-Kabat) | CDR1 (SEQ ID NO: 76)- |
| | (SEQ ID NO: 71) or SYAMS (SEQ ID | RASQGIDSWLA |
| | NO: 115) | CDR2 (SEQ ID NO: 77)-AASSLQS |
| | CDR2 (SEQ ID NO: 72)- | CDR3 (SEQ ID NO: 78)- |
| | AISGSGGSTYYADSVKG | QQGVSYPRT |
| | CDR3-AKDGGYYDSGAGDY (non- | |
| | Kabat) (SEQ ID NO: 73) or | |
| | DGGYYDSGAGDY (SEQ ID NO: 74) | |
| ADI- | EVQLVESGGGLVKPGGSLRLSCAA | DIQMTQSPSSVSASVGDRVTITCR |
| 27749 | SGFTFSSYSMNWVRQAPGKGLEWV | ASQGISSWLAWYQQKPGKAPKLL |
| (A49) | SSISSSSSYIYYADSVKGRFTISRDN | IYAASSLQSGVPSRFSGSGSGTDFT |
| | AKNSLYLQMNSLRAEDTAVYYCA | LTISSLQPEDFATYYCQQGVSFPRT |
| | RGAPMGAAAGWFDPWGQGTLVTV | FGGGTKVEIK |
| | SS | (SEQ ID NO: 85) |
| | (SEQ ID NO: 79) | CDR1 (SEQ ID NO: 86)- |
| | CDR1-FTFSSYSMN (SEQ ID NO: 80) | RASQGISSWLA |
| | (non-Kabat) or SYSMN (SEQ ID | CDR2 (SEQ ID NO: 77)-AASSLQS |
| | NO: 81) | CDR3 (SEQ ID NO: 87)- |
| | CDR2 (SEQ ID NO: 82)- | QQGVSFPRT |
| | SISSSSSYIYYADSVKG | |
| | CDR3-ARGAPMGAAAGWFDP | |
| | (SEQ ID NO: 83) (non-Kabat) or | |
| | GAPMGAAAGWFDP (SEQ ID NO: 84) | |
| | scFv (VL-VH) with Q44C in VH and G100C in VL, linker italicized: | |
| | DIQMTQSPSSVSASVGDRVTITCRASQGISSWLAWYQQKPGKAPKLLIYA | |
| | ASSLQSGVPSRFSGSGSGTDFTLTISSLQPEDFATYYCQQGVSFPRTFGCGT | |
| | KVEIKGGGGSGGGGSGGGGSGGGGSEVQLVESGGGLVKPGGSLRLSCAAS | |
| | GFTFSSYSMNWVRQAPGKCLEWVSSISSSSSYIYYADSVKGRFTISRDNAK | |
| | NSLYLQMNSLRAEDTAVYYCARGAPMGAAAGWFDPWGQGTLVTVSS | |
| | (SEQ ID NO: 88) | |
| ADI- | QVQLVQSGAEVKKPGASVKVSCKA | EIVLTQSPATLSLSPGERATLSCRA |
| 29378 | SGYTFTSYYMHWVRQAPGQGLEW | SQSVSSYLAWYQQKPGQAPRLLIY |
| (E78) | MGIINPSGGSTSYAQKFQGRVTMTR | DASNRATGIPARFSGSGSGTDFTL |
| | DTSTSTVYMELSSLRSEDTAVYYCA | TISSLEPEDFAVYYCQQSDNWPFT |
| | REGAGFAYGMDYYYMDVWGKGT | FGGGTKVEIK |
| | TVTVSS | (SEQ ID NO: 92) |
| | (SEQ ID NO: 89) | CDR1 (SEQ ID NO: 93)- |
| | CDR1-YTFTSYYMH (SEQ ID | RASQSVSSYLA |
| | NO: 53) (non-Kabat) or SYYMH (SEQ | CDR2 (SEQ ID NO: 44)-DASNRAT |
| | ID NO: 54) | CDR3 (SEQ ID NO: 94)- |
| | CDR2 (SEQ ID NO: 55)- | QQSDNWPFT |
| | IINPSGGSTSYAQKFQG | |
| | CDR3- | |
| | AREGAGFAYGMDYYYMDV (SEQ | |
| | ID NO: 90) (non-Kabat) or | |
| | EGAGFAYGMDYYYMDV (SEQ ID | |
| | NO: 91) | |
| A49MI | EVQLVESGGGLVKPGGSLRLSCAA | DIQMTQSPSSVSASVGDRVTITCR |
| | SGFTFSSYSMNWVRQAPGKGLEWV | ASQGISSWLAWYQQKPGKAPKLL |
| | SSISSSSSYIYYADSVKGRFTISRDN | IYAASSLQSGVPSRFSGSGSGTDFT |
| | AKNSLYLQMNSLRAEDTAVYYCA | LTISSLQPEDFATYYCQQGVSFPRT |
| | RGAPIGAAAGWFDPWGQGTLVTVS | FGGGTKVEIK |
| | S (SEQ ID NO: 95) | (SEQ ID NO: 85) |
| | CDR1: FTFSSYSMN (SEQ ID NO: 80) | CDR1 (SEQ ID NO: 86)- |
| | (non-Kabat) or SYSMN (SEQ ID | RASQGISSWLA |
| | NO: 81) | CDR2 (SEQ ID NO: 77)-AASSLQS |
| | CDR2: SISSSSSYIYYADSVKG | CDR3 (SEQ ID NO: 87)- |
| | (SEQ ID NO: 82) | QQGVSFPRT |
| | CDR3: ARGAPIGAAAGWFDP (SEQ | |
| | ID NO: 96) (non-Kabat) or | |
| | GAPIGAAAGWFDP | |
| | (SEQ ID NO: 97) | |
| | scFv (VL-VH) with Q44C in VH and G100C in VL, linker italicized: | |
| | DIQMTQSPSSVSASVGDRVTITCRASQGISSWLAWYQQKPGKAPKLLIYA | |
| | ASSLQSGVPSRFSGSGSGTDFTLTISSLQPEDFATYYCQQGVSFPRTFGCGT | |
| | KVEIKGGGGSGGGGSGGGGSGGGGSEVQLVESGGGLVKPGGSLRLSCAAS | |
| | GFTFSSYSMNWVRQAPGKCLEWVSSISSSSSYIYYADSVKGRFTISRDNAK | |
| | NSLYLQMNSLRAEDTAVYYCARGAPIGAAAGWFDPWGQGTLVTVSS | |
| | (SEQ ID NO: 275) | |
| A49MQ | EVQLVESGGGLVKPGGSLRLSCAA | DIQMTQSPSSVSASVGDRVTITCR |
| | SGFTFSSYSMNWVRQAPGKGLEWV | ASQGISSWLAWYQQKPGKAPKLL |
| | SSISSSSSYIYYADSVKGRFTISRDN | IYAASSLQSGVPSRFSGSGSGTDFT |
| | AKNSLYLQMNSLRAEDTAVYYCA | LTISSLQPEDFATYYCQQGVSFPRT |
| | RGAPQGAAAGWFDPWGQGTLVTV | FGGGTKVEIK |
| | SS | (SEQ ID NO: 85) |
| | (SEQ ID NO: 98) | CDR1 (SEQ ID NO: 86)- |
| | CDR1: FTFSSYSMN (SEQ ID NO: 80) | RASQGISSWLA |
| | (non-Kabat) or SYSMN (SEQ ID | CDR2 (SEQ ID NO: 77)-AASSLQS |
| | NO: 81) | CDR3 (SEQ ID NO: 87)- |
| | CDR2: SISSSSSYIYYADSVKG | QQGVSFPRT |
| | (SEQ ID NO: 82) | |
| | CDR3-ARGAPQGAAAGWFDP (SEQ | |
| | ID NO: 99) (non-Kabat) or | |
| | GAPQGAAAGWFDP (SEQ ID | |
| | NO: 100) | |
| A49ML | EVQLVESGGGLVKPGGSLRLSCAA | DIQMTQSPSSVSASVGDRVTITCR |
| | SGFTFSSYSMNWVRQAPGKGLEWV | ASQGISSWLAWYQQKPGKAPKLL |
| | SSISSSSSYIYYADSVKGRFTISRDN | IYAASSLQSGVPSRFSGSGSGTDFT |
| | AKNSLYLQMNSLRAEDTAVYYCA | LTISSLQPEDFATYYCQQGVSFPRT |
| | RGAPLGAAAGWFDPWGQGTLVTV | FGGGTKVEIK |
| | SS | (SEQ ID NO: 85) |
| | (SEQ ID NO: 101) | CDR1 (SEQ ID NO: 86)- |
| | CDR1: FTFSSYSMN (SEQ ID NO: 80) | RASQGISSWLA |
| | (non-Kabat) or SYSMN (SEQ ID | CDR2 (SEQ ID NO: 77)-AASSLQS |
| | NO: 81) | CDR3 (SEQ ID NO: 87)- |
| | CDR2: SISSSSSYIYYADSVKG | QQGVSFPRT |
| | (SEQ ID NO: 82) | |
| | CDR3-ARGAPLGAAAGWFDP | |
| | (SEQ ID NO: 102) (non-Kabat) or | |
| | GAPLGAAAGWFDP (SEQ ID | |
| | NO: 103) | |
| A49MF | EVQLVESGGGLVKPGGSLRLSCAA | DIQMTQSPSSVSASVGDRVTITCR |
| | SGFTFSSYSMNWVRQAPGKGLEWV | ASQGISSWLAWYQQKPGKAPKLL |
| | SSISSSSSYIYYADSVKGRFTISRDN | IYAASSLQSGVPSRFSGSGSGTDFT |
| | AKNSLYLQMNSLRAEDTAVYYCA | LTISSLQPEDFATYYCQQGVSFPRT |
| | RGAPFGAAAGWFDPWGQGTLVTV | FGGGTKVEIK |
| | SS | (SEQ ID NO: 85) |
| | (SEQ ID NO: 104) | CDR1 (SEQ ID NO: 86)- |
| | CDR1: FTFSSYSMN (SEQ ID NO: 80) | RASQGISSWLA |
| | (non-Kabat) or SYSMN (SEQ ID | CDR2 (SEQ ID NO: 77)-AASSLQS |
| | NO: 81) | CDR3 (SEQ ID NO: 87)- |
| | CDR2: SISSSSSYIYYADSVKG | QQGVSFPRT |
| | (SEQ ID NO: 82) | |
| | CDR3-ARGAPFGAAAGWFDP (SEQ | |
| | ID NO: 105) (non-Kabat) or | |
| | GAPFGAAAGWFDP (SEQ ID | |
| | NO: 106) | |
| A49MV | EVQLVESGGGLVKPGGSLRLSCAA | DIQMTQSPSSVSASVGDRVTITCR |
| | SGFTFSSYSMNWVRQAPGKGLEWV | ASQGISSWLAWYQQKPGKAPKLL |
| | SSISSSSSYIYYADSVKGRFTISRDN | IYAASSLQSGVPSRFSGSGSGTDFT |
| | AKNSLYLQMNSLRAEDTAVYYCA | LTISSLQPEDFATYYCQQGVSFPRT |
| | RGAPVGAAAGWFDPWGQGTLVTV | FGGGTKVEIK |
| | SS | (SEQ ID NO: 85) |
| | (SEQ ID NO: 107) | CDR1 (SEQ ID NO: 86)- |
| | CDR1: FTFSSYSMN (SEQ ID NO: 80) | RASQGISSWLA |
| | (non-Kabat) or SYSMN (SEQ ID | CDR2 (SEQ ID NO: 77)-AASSLQS |
| | NO: 81) | CDR3 (SEQ ID NO: 87)- |
| | CDR2: SISSSSSYIYYADSVKG | QQGVSFPRT |
| | (SEQ ID NO: 82) | |
| | CDR3-ARGAPVGAAAGWFDP (SEQ | |
| | ID NO: 108) (non-Kabat) or | |
| | GAPVGAAAGWFDP (SEQ ID | |
| | NO: 109) | |
| A49- | EVQLVESGGGLVKPGGSLRLSCAA | DIQMTQSPSSVSASVGDRVTITCR |
| consensus | SGFTFSSYSMNWVRQAPGKGLEWV | ASQGISSWLAWYQQKPGKAPKLL |
| | SSISSSSSYIYYADSVKGRFTISRDN | IYAASSLQSGVPSRFSGSGSGTDFT |
| | AKNSLYLQMNSLRAEDTAVYYCA | LTISSLQPEDFATYYCQQGVSFPRT |
| | RGAPXGAAAGWFDPWGQGTLVTV | FGGGTKVEIK |
| | SS, wherein X is M, L, I, V, Q, | (SEQ ID NO: 85) |
| | or F (SEQ ID NO: 110) | CDR1 (SEQ ID NO: 86)- |
| | CDR1: FTFSSYSMN (SEQ ID NO: 80) | RASQGISSWLA |
| | (non-Kabat) or SYSMN (SEQ ID | CDR2 (SEQ ID NO: 77)-AASSLQS |
| | NO: 81) | CDR3 (SEQ ID NO: 87)- |
| | CDR2: SISSSSSYIYYADSVKG | QQGVSFPRT |
| | (SEQ ID NO: 82) | |
| | CDR3-ARGAPXGAAAGWFDP (SEQ | |
| | ID NO: 111) (non-Kabat) or | |
| | GAPXGAAAGWFDP (SEQ ID | |
| | NO: 112), wherein X is M, L, I, V, | |
| | Q, or F | |
| NKG2D | QVQLVESGGGLVKPGGSLRLSCAA | QSALTQPASVSGSPGQSITISCSGSS |
| binder in | SGFTFSSYGMHWVRQAPGKGLEW | SNIGNNAVNWYQQLPGKAPKLLI |
| U.S. Pat. | VAFIRYDGSNKYYADSVKGRFTISR | YYDDLLPSGVSDRFSGSKSGTSAF |
| No. | DNSKNTLYLQMNSLRAEDTAVYYC | LAISGLQSEDEADYYCAAWDDSL |
| 9,273,136 | AKDRGLGDGTYFDYWGQGTTVTV | NGPVFGGGTKLTVL (SEQ ID |
| | SS (SEQ ID NO: 113) | NO: 114) |
| NKG2D | QVHLQESGPGLVKPSETLSLTCTVS | EIVLTQSPGTLSLSPGERATLSCRA |
| binder in | DDSISSYYWSWIRQPPGKGLEWIGH | SQSVSSSYLAWYQQKPGQAPRLLI |
| U.S. Pat. | ISYSGSANYNPSLKSRVTISVDTSKN | YGASSRATGIPDRFSGSGSGTDFTL |
| No. | QFSLKLSSVTAADTAVYYCANWDD | TISRLEPEDFAVYYCQQYGSSPWT |
| 7,879,985 | AFNIWGQGTMVTVSS (SEQ ID | FGQGTKVEIK (SEQ ID NO: 117) |
| | NO: 116) | |

In certain embodiments, the first antigen-binding site that binds NKG2D (e.g. human NKG2D) comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 1, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in Table 1. In certain embodiments, the first antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences of an antibody discloses in Table 1. In certain embodiments, the first antigen-binding site comprises the heavy chain CDR1, CDR2, and CDR3 and the light chain CDR1, CDR2, and CDR3 of an antibody disclosed in Table 1.

In certain embodiments, the first antigen-binding site that binds to NKG2D comprises a heavy chain variable domain derived from SEQ ID NO:1, such as by having an amino acid sequence at least 90% (e.g., at least 90%, at least 91%, at least 92%, at least 93%, at least 94%, at least 95%, at least 96%, at least 97%, at least 98%, at least 99%, or 100%) identical to SEQ ID NO:1, and/or incorporating amino acid sequences identical to the CDR1 (SEQ ID NO:2), CDR2 (SEQ ID NO:3), and CDR3 (SEQ ID NO:4) sequences of SEQ ID NO:1. The heavy chain variable domain related to SEQ ID NO:1 can be coupled with a variety of light chain variable domains to form an NKG2D binding site. For example, the first antigen-binding site that incorporates a heavy chain variable domain related to SEQ ID NO:1 can further incorporate a light chain variable domain selected from the sequences derived from SEQ ID NOs: 5, 6, 7, 8, 9, 12, 13, 14, 15, 16, 17, 18, 19, 20, 21, 22, 23, 24, 25, and 46. For example, the first antigen-binding site incorporates a heavy chain variable domain with amino acid sequences at least 90% (e.g., at least 90%, at least 91%, at least 92%, at least 93%, at least 94%, at least 95%, at least 96%, at least 97%, at least 98%, at least 99%, or 100%) identical to SEQ ID NO:1 and a light chain variable domain with amino acid sequences at least 90% (e.g., at least 90%, at least 91%, at least 92%, at least 93%, at least 94%, at least 95%, at least 96%, at least 97%, at least 98%, at least 99%, or 100%) identical to any one of the sequences selected from SEQ ID NOs: 5, 6, 7, 8, 9, 12, 13, 14, 15, 16, 17, 18, 19, 20, 21, 22, 23, 24, 25, and 46.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:26, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:32. In certain embodiments, the VH comprises CDR1, CDR2, and CDR3 comprising the amino acid sequences of SEQ ID NOs: 27 or 28, 29, and 30 or 31, respectively (e.g., SEQ ID NOs: 27, 29, and 30, respectively, or SEQ ID NOs: 28, 29, and 31, respectively). In certain embodiments, the VL comprises CDR1, CDR2, and CDR3 comprising the amino acid sequences of SEQ ID NOs: 33, 34, and 35, respectively. In certain embodiments, the first antigen-binding site comprises (a) a VH that comprises CDR1, CDR2, and CDR3 comprising the amino acid sequences of SEQ ID NOs: 27 or 28, 29, and 30 or 31, respectively (e.g., SEQ ID NOs: 27, 29, and 30, respectively, or SEQ ID NOs: 28, 29, and 31, respectively); and (b) a VL that comprises CDR1, CDR2, and CDR3 comprising the amino acid sequences of SEQ ID NOs: 33, 34, and 35, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:36, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 37 or 38, 39, and 40 or 41, respectively (e.g., SEQ ID NOs: 37, 39, and 40, respectively, or SEQ ID NOs: 38, 39, and 41, respectively). In certain embodiments, the VL comprises CDR1, CDR2, and CDR3 comprising the amino acid sequences of SEQ ID NOs: 43, 44, and 45, respectively. In certain embodiments, the first antigen-binding site comprises (a) a VH that comprises CDR1, CDR2, and CDR3 comprising the amino acid sequences of SEQ ID NOs: 37 or 38, 39, and 40 or 41, respectively (e.g., SEQ ID NOs: 37, 39, and 40, respectively, or SEQ ID NOs: 38, 39, and 41, respectively); and (b) a VL that comprises CDR1, CDR2, and CDR3 comprising the amino acid sequences of SEQ ID NOs: 43, 44, and 45, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:47, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:49. In certain embodiments, the VH comprises CDR1, CDR2, and CDR3 comprising the amino acid sequences of SEQ ID NOs: 27, 29, and 48, respectively. In certain embodiments, the VL comprises CDR1, CDR2, and CDR3 comprising the amino acid sequences of SEQ ID NOs: 50, 34, and 51, respectively. In certain embodiments, the first antigen-binding site comprises (a) a VH that comprises CDR1, CDR2, and CDR3 comprising the amino acid sequences of SEQ ID NOs: 27, 29, and 48, respectively; and (b) a VL that comprises CDR1, CDR2, and CDR3 comprising the amino acid sequences of SEQ ID NOs: 50, 34, and 51, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:52, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:58. In certain embodiments, the VH comprises CDR1, CDR2, and CDR3 comprising the amino acid sequences of SEQ ID NOs: 53 or 54, 55, and 56 or 57, respectively (e.g., SEQ ID NOs: 53, 55, and 56, respectively, or SEQ ID NOs: 54, 55, and 57, respectively). In certain embodiments, the VL comprises CDR1, CDR2, and CDR3 comprising the amino acid sequences of SEQ ID NOs: 59, 60, and 61, respectively. In certain embodiments, the first antigen-binding site comprises (a) a VH that comprises CDR1, CDR2, and CDR3 comprising the amino acid sequences of SEQ ID NOs: 53 or 54, 55, and 56 or 57, respectively (e.g., SEQ ID NOs: 53, 55, and 56, respectively, or SEQ ID NOs: 54, 55, and 57, respectively); and (b) a VL that comprises CDR1, CDR2, and CDR3 comprising the amino acid sequences of SEQ ID NOs: 59, 60, and 61, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:62, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:68. In certain embodiments, the VH comprises CDR1, CDR2, and CDR3 comprising the amino acid sequences of SEQ ID NOs: 63 or 64, 65, and 66 or 67, respectively (e.g., SEQ ID NOs: 63, 65, and 66, respectively, or SEQ ID NOs: 64, 65, and 67, respectively). In certain embodiments, the VL comprises CDR1, CDR2, and CDR3 comprising the amino acid sequences of SEQ ID NOs: 59, 60, and 69, respectively. In certain embodiments, the first antigen-binding site comprises (a) a VH that comprises CDR1, CDR2, and CDR3 comprising the amino acid sequences of SEQ ID NOs: 63 or 64, 65, and 66 or 67, respectively (e.g., SEQ ID NOs: 63, 65, and 66, respectively, or SEQ ID NOs: 64, 65, and 67, respectively); and (b) a VL that comprises CDR1, CDR2, and CDR3 comprising the amino acid sequences of SEQ ID NOs: 59, 60, and 69, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:89, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:92. In certain embodiments, the VH comprises CDR1, CDR2, and CDR3 comprising the amino acid sequences of SEQ ID NOs: 53 or 54, 55, and 90 or 91, respectively (e.g., SEQ ID NOs: 53, 55, and 90, respectively, or SEQ ID NOs: 54, 55, and 91, respectively). In certain embodiments, the VL comprises CDR1, CDR2, and CDR3 comprising the amino acid sequences of SEQ ID NOs: 93, 44, and 94, respectively. In certain embodiments, the first antigen-binding site comprises (a) a VH that comprises CDR1, CDR2, and CDR3 comprising the amino acid sequences of SEQ ID NOs: 53 or 54, 55, and 90 or 91, respectively (e.g., SEQ ID NOs: 53, 55, and 90, respectively, or SEQ ID NOs: 54, 55, and 91, respectively); and (b) a VL that comprises CDR1, CDR2, and CDR3 comprising the amino acid sequences of SEQ ID NOs: 93, 44, and 94, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:70, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:75. In certain embodiments, the VH comprises CDR1, CDR2, and CDR3 comprising the amino acid sequences of SEQ ID NOs: 71 or 115, 72, and 73 or 74, respectively (e.g., SEQ ID NOs: 71, 72, and 73, respectively, or SEQ ID NOs: 115, 72, and 74, respectively). In certain embodiments, the VL comprises CDR1, CDR2, and CDR3 comprising the amino acid sequences of SEQ ID NOs: 76, 77, and 78, respectively. In certain embodiments, the first antigen-binding site comprises (a) a VH that comprises CDR1, CDR2, and CDR3 comprising the amino acid sequences of SEQ ID NOs: 71 or 115, 72, and 73 or 74, respectively (e.g., SEQ ID NOs: 71, 72, and 73, respectively, or SEQ ID NOs: 115, 72, and 74, respectively); and (b) a VL that comprises CDR1, CDR2, and CDR3 comprising the amino acid sequences of SEQ ID NOs: 76, 77, and 78, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:79, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 83 or 84, respectively (e.g., SEQ ID NOs: 80, 82, and 83, respectively, or SEQ ID NOs: 81, 82, and 84, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 83 or 84 respectively (e.g., SEQ ID NOs: 80, 82, and 83, respectively, or SEQ ID NOs: 81, 82, and 84, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:95, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 96 or 97, respectively (e.g., SEQ ID NOs: 80, 82, and 96, respectively, or SEQ ID NOs: 81, 82, and 97, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 96 or 97, respectively (e.g., SEQ ID NOs: 80, 82, and 96, respectively, or SEQ ID NOs: 81, 82, and 97, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:98, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 99 or 100, respectively (e.g., SEQ ID NOs: 80, 82, and 99, respectively, or SEQ ID NOs: 81, 82, and 100, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 99 or 100, respectively (e.g., SEQ ID NOs: 80, 82, and 99, respectively, or SEQ ID NOs: 81, 82, and 100, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:101, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 102 or 103, respectively (e.g., SEQ ID NOs: 80, 82, and 102, respectively, or SEQ ID NOs: 81, 82, and 103, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 102 or 103, respectively (e.g., SEQ ID NOs: 80, 82, and 102, respectively, or SEQ ID NOs: 81, 82, and 103, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:104, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 105 or 106, respectively (e.g., SEQ ID NOs: 80, 82, and 105, respectively, or SEQ ID NOs: 81, 82, and 106, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 105 or 106, respectively (e.g., SEQ ID NOs: 80, 82, and 105, respectively, or SEQ ID NOs: 81, 82, and 106, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:107, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 108 or 109, respectively (e.g., SEQ ID NOs: 80, 82, and 108, respectively, or SEQ ID NOs: 81, 82, and 109, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 108 or 109, respectively (e.g., SEQ ID NOs: 80, 82, and 108, respectively, or SEQ ID NOs: 81, 82, and 109, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:110, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the VH comprises CDR1, CDR2, and CDR3 comprising the amino acid sequences of SEQ ID NOs: 80 or 81, 82, and 111 or 112, respectively (e.g., SEQ ID NOs: 80, 82, and 111, respectively, or SEQ ID NOs: 81, 82, and 112, respectively). In certain embodiments, the VL comprises CDR1, CDR2, and CDR3 comprising the amino acid sequences of SEQ ID NOs: 86, 77, and 87, respectively. In certain embodiments, the first antigen-binding site comprises (a) a VH that comprises CDR1, CDR2, and CDR3 comprising the amino acid sequences of SEQ ID NOs: 80 or 81, 82, and 111 or 112, respectively (e.g., SEQ ID NOs: 80, 82, and 111, respectively, or SEQ ID NOs: 81, 82, and 112, respectively); and (b) a VL that comprises CDR1, CDR2, and CDR3 comprising the amino acid sequences of SEQ ID NOs: 86, 77, and 87, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:113, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:114.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:116, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:117.

The multispecific binding proteins can bind to NKG2D-expressing cells, which include but are not limited to NK cells, γδ T cells and CD8⁺ αβ T cells. Upon NKG2D binding, the multispecific binding proteins may block natural ligands, such as ULBP6 and MICA, from binding to NKG2D and activating NK cells.

The multispecific binding proteins binds to cells expressing CD16, an Fc receptor on the surface of leukocytes including natural killer cells, macrophages, neutrophils, eosinophils, mast cells, and follicular dendritic cells. A protein of the present disclosure binds to NKG2D with an affinity of K_{D} of 2 nM to 120 nM, e.g., 2 nM to 110 nM, 2 nM to 100 nM, 2 nM to 90 nM, 2 nM to 80 nM, 2 nM to 70 nM, 2 nM to 60 nM, 2 nM to 50 nM, 2 nM to 40 nM, 2 nM to 30 nM, 2 nM to 20 nM, 2 nM to 10 nM, about 15 nM, about 14 nM, about 13 nM, about 12 nM, about 11 nM, about 10 nM, about 9 nM, about 8 nM, about 7 nM, about 6 nM, about 5 nM, about 4.5 nM, about 4 nM, about 3.5 nM, about 3 nM, about 2.5 nM, about 2 nM, about 1.5 nM, about 1 nM, between about 0.5 nM to about 1 nM, about 1 nM to about 2 nM, about 2 nM to 3 nM, about 3 nM to 4 nM, about 4 nM to about 5 nM, about 5 nM to about 6 nM, about 6 nM to about 7 nM, about 7 nM to about 8 nM, about 8 nM to about 9 nM, about 9 nM to about 10 nM, about 1 nM to about 10 nM, about 2 nM to about 10 nM, about 3 nM to about 10 nM, about 4 nM to about 10 nM, about 5 nM to about 10 nM, about 6 nM to about 10 nM, about 7 nM to about 10 nM, or about 8 nM to about 10 nM. In some embodiments, NKG2D-binding sites bind to NKG2D with a K_{D} of 10 to 62 nM.

### BAFF-R Binding Site

The BAFF-R site of the multispecific binding protein disclosed herein comprises a heavy chain variable domain and a light chain variable domain.

In one aspect, the present disclosure provides multispecific binding proteins that bind to the NKG2D receptor and CD16 receptor on natural killer cells, and BAFF-R. Table 2 lists some exemplary sequences of heavy chain variable domains and light chain variable domains that, in combination, can bind to BAFF-R.

CDR sequences are identified under Chothia numbering unless otherwise indicated.

TABLE 2

| | Heavy chain variable | Light chain variable |
|---|---|---|
| | domain amino | domain amino |
| Source | acid sequence | acid sequence |
| Ianalumab | QVQLQQSGPGLVKPSQTLSLTCAIS | DIVLTQSPATLSLSPGERATLSCRA |
| (Morpho | GDSVSSNSAAWGWIRQSPGRGLEW | SQFILPEYLSWYQQKPGQAPRLLI |
| Sys/ | LGRIYYRSKWYNSYAVSVKSRITIN | YGSSSRATGVPARFSGSGSGTDFT |
| Novartis) | PDTSKNQFSLQLNSVTPEDTAVYYC | LTISSLEPEDFAVYYCQQFYSSPLT |
| | ARYQWVPKIGVFDSWGQGTLVTVS | FGQGTKVEIK (SEQ ID NO: 146) |
| | S (SEQ ID NO: 145) | CDR1: RASQFILPEYLS |
| | CDR1: SNSAAWG (SEQ ID NO: 157) | (SEQ ID NO: 160) (Kabat) |
| | (Kabat) or SSNSAAWG (SEQ ID | CDR2: GSSSRAT (SEQ ID NO: 161) |
| | NO: 135) | (Kabat) |
| | CDR2: RIYYRSKWYNSYAVSVKS | CDR3: QQFYSSPLT |
| | (SEQ ID NO: 158) (Kabat) or | (SEQ ID NO: 162) (Kabat) |
| | WLGRIYYRSKWYNS (SEQ ID | |
| | NO: 136) | |
| | CDR3: YQWVPKIGVFDS | |
| | (SEQ ID NO: 159) (Kabat) or | |
| | ARYQWVPKIGVFD (SEQ ID | |
| | NO: 137) | |
| scFv | VL-VH (SEQ ID NO: 207) | |
| derived | DIVLTQSPATLSLSPGERATLSCRASQFILPEYLSWYQQKPGQAPRLLIY | |
| from | GSSSRATGVPARFSGSGSGTDFTLTISSLEPEDFAVYYCQQFYSSPLTFG | |
| ianalumab | CGTKVEIKGGGGSGGGGSGGGGSGGGGSQVQLQQSGPGLVKPSQTLSLTC | |
| (linker | AISGDSVSSNSAAWGWIRQSPGRCLEWLGRIYYRSKWYNSYAVSVKSRIT | |
| italicized) | INPDTSKNQFSLQLNSVTPEDTAVYYCARYQWVPKIGVFDSWGQGTLVTV | |
| | SS | |
| | VH-VL (SEQ ID NO: 138) | |
| | QVQLQQSGPGLVKPSQTLSLTCAISGDSVSSNSAAWGWIRQSPGRCLEWL | |
| | GRIYYRSKWYNSYAVSVKSRITINPDTSKNQFSLQLNSVTPEDTAVYYCA | |
| | RYQWVPKIGVFDSWGQGTLVTVSSGGGGSGGGGSGGGGSGGGGSDIVLTQ | |
| | SPATLSLSPGERATLSCRASQFILPEYLSWYQQKPGQAPRLLIYGSSSRA | |
| | TGVPARFSGSGSGTDFTLTISSLEPEDFAVYYCQQFYSSPLTFGCGTKVE | |
| | IK | |
| V3-46s | EVQLVESGGGLVQPGGSLRLSCAA | DIQMTQSPSSLSASVGDRVTITCR |
| (Genentech) | SGFTISSSSIHWVRQAPGKGLEWVA | ASQDVSTAVAWYQQKPGKAPKL |
| (WO2006 | WVLPSVGFTDYADSVKGRFTISAD | LIYSASFLYSGVPSRFSGSGSGTDF |
| 073941) | TSKNTAYLQMNSLRAEDTAVYYC | TLTISSLQPEDFATYYCQQSQISPP |
| | ARRVCYNRLGVCAGGMDYWGQG | TFGQGTKVEIK (SEQ ID NO: 148) |
| | TLVTVSS (SEQ ID NO: 147) | CDR1: QDVSTA (SEQ ID NO: 166) |
| | CDR1: TISSSS (SEQ ID NO: 163) | (Kabat) |
| | (Kabat) | CDR2: YSASFLY (SEQ ID NO: 167) |
| | CDR2: AWVLPSVGFTD | (Kabat) |
| | (SEQ ID NO: 164) (Kabat) | CDR3: CQQSQIS (SEQ ID NO: 168) |
| | CDR3: RVCYNRLGVCAGG | (Kabat) |
| | (SEQ ID NO: 165) (Kabat) | |
| scFv | VL-VH (SEQ ID NO: 139) | |
| derived | DIQMTQSPSSLSASVGDRVTITCRASQDVSTAVAWYQQKPGKAPKLLIYS | |
| from V3- | ASFLYSGVPSRFSGSGSGTDFTLTISSLQPEDFATYYCQQSQISPPTFGC | |
| 46s | GTKVEIKGGGGSGGGGSGGGGSGGGGSEVQLVESGGGLVQPGGSLRLSCA | |
| (linker | ASGFTISSSSIHWVRQAPGKCLEWVAWVLPSVGFTDYADSVKGRFTISAD | |
| italicized) | TSKNTAYLQMNSLRAEDTAVYYCARRVCYNRLGVCAGGMDYWGQGTLVTV | |
| | SS | |
| | VH-VL (SEQ ID NO: 140) | |
| | EVQLVESGGGLVQPGGSLRLSCAASGFTISSSSIHWVRQAPGKCLEWVAW | |
| | VLPSVGFTDYADSVKGRFTISADTSKNTAYLQMNSLRAEDTAVYYCARR | |
| | VCYNRLGVCAGGMDYWGQGTLVTVSSGGGGSGGGGSGGGGSGGGGSDIQ | |
| | MTQSPSSLSASVGDRVTITCRASQDVSTAVAWYQQKPGKAPKLLIYSAS | |
| | FLYSGVPSRFSGSGSGTDFTLTISSLQPEDFATYYCQQSQISPPTFGCG | |
| | TKVEIK | |
| V3-46s- | EVQLVESGGGLVQPGGSLRLSCAA | DIQMTQSPSSLSASVGDRVTITCR |
| 42 | SGFTISSSSIHWVRQAPGKGLEWVA | ASEDISTAVAWYQQKPGKAPKLLI |
| (Genentech) | WVLPSVGFTDYADSVKGRFTISAD | YAASFLYSGVPSRFSGSGSGTDFT |
| (WO2006 | TSKNTAYLQMNSLRAEDTAVYYC | LTISSLQPEDFATYYCQQSQISPPTF |
| 073941) | ARRVCYNRLGVCAGGMDYWGQG | GQGTKVEIK (SEQ ID NO: 150) |
| | TLVTVSS (SEQ ID NO: 147) | CDR1: EDISTA (SEQ ID NO: 169) |
| | CDR1: TISSSS (SEQ ID NO: 163) | (Kabat) |
| | (Kabat) | CDR2: YAASFLY (SEQ ID NO: 170) |
| | CDR2: AWVLPSVGFTD | (Kabat) |
| | (SEQ ID NO: 164) (Kabat) | CDR3: CQQSQIS (SEQ ID NO: 168) |
| | CDR3: RVCYNRLGVCAGG | (Kabat) |
| | (SEQ ID NO: 165) (Kabat) | |
| scFv | VL-VH (SEQ ID NO: 141) | |
| derived | DIQMTQSPSSLSASVGDRVTITCRASEDISTAVAWYQQKPGKAPKLLIYAA | |
| from V3- | SFLYSGVPSRFSGSGSGTDFTLTISSLQPEDFATYYCQQSQISPPTFGCGT | |
| 46s-42 | KVEIKGGGGSGGGGSGGGGSGGGGSEVQLVESGGGLVQPGGSLRLSCAASG | |
| (linker | FTISSSSIHWVRQAPGKCLEWVAWVLPSVGFTDYADSVKGRFTISADTSKN | |
| italicized) | TAYLQMNSLRAEDTAVYYCARRVCYNRLGVCAGGMDYWGQGTLVTVS | |
| | S | |
| | VH-VL (SEQ ID NO: 142) | |
| | EVQLVESGGGLVQPGGSLRLSCAASGFTISSSSIHWVRQAPGKCLEWVAW | |
| | VLPSVGFTDYADSVKGRFTISADTSKNTAYLQMNSLRAEDTAVYYCARRV | |
| | CYNRLGVCAGGMDYWGQGTLVTVSSGGGGSGGGGSGGGGSGGGGSDIQMT | |
| | QSPSSLSASVGDRVTITCRASEDISTAVAWYQQKPGKAPKLLIYAASFLY | |
| | SGVPSRFSGSGSGTDFTLTISSLQPEDFATYYCQQSQISPPTFGCGTKVE | |
| | IK | |
| Hu9.1-73 | EVQLVESGGGLVQPGGSLRLSCAA | DIQMTQSPSSLSASVGDRVTITCKS |
| (Genentech) | SGLPMAGFYTSWVRQAPGKGLEW | SQSLLYSSNQNNYLAWYQQKPGK |
| (WO2006 | VGFIRDKANGYTTEYNPSVKGRFTI | APKLLIYWAQHLDSGVPSRFSGSG |
| 073941) | SADTSKNTAYLQMNSLRAEDTAVY | SGTDFTLTISSLQPEDFATYYCQQ |
| | YCAQVRRALDYWGQGTLVTVSS | YYTYPYTFGQGTKVEIK |
| | (SEQ ID NO: 151) | (SEQ ID NO: 152) |
| | CDR1: GFYTS (SEQ ID NO: 171) | CDR1: KSSQSLLYSSNONNYLA |
| | (Kabat) | (SEQ ID NO: 174) (Kabat) |
| | CDR2: FIRDKANGYTTEYNPSVKG | CDR2: WAQHLDS (SEQ ID NO: 175) |
| | (SEQ ID NO: 172) (Kabat) | (Kabat) |
| | CDR3: VRRALDY (SEQ ID NO: 173) | CDR3: QQYYTYPYT |
| | (Kabat) | (SEQ ID NO: 176) (Kabat) |
| scFv | VL-VH (SEQ ID NO: 143) | |
| derived | DIQMTQSPSSLSASVGDRVTITCKSSQSLLYSSNONNYLAWYQQKPGKA | |
| from | PKLLIYWAQHLDSGVPSRFSGSGSGTDFTLTISSLQPEDFATYYCQQYY | |
| Hu9.1-73 | TYPYTFGCGTKVEIKGGGGSGGGGSGGGGSGGGGSEVQLVESGGGLVQP | |
| (linker | GGSLRLSCAASGLPMAGFYTSWVRQAPGKCLEWVGFIRDKANGYTTEYN | |
| italicized) | PSVKGRFTISADTSKNTAYLQMNSLRAEDTAVYYCAQVRRALDYWGQGT | |
| | LVTVSS | |
| | VH-VL (SEQ ID NO: 144) | |
| | EVQLVESGGGLVQPGGSLRLSCAASGLPMAGFYTSWVRQAPGKCLEWVG | |
| | FIRDKANGYTTEYNPSVKGRFTISADTSKNTAYLQMNSLRAEDTAVYYC | |
| | AQVRRALDYWGQGTLVTVSSGGGGSGGGGSGGGGSGGGGSDIQMTQSPS | |
| | SLSASVGDRVTITCKSSQSLLYSSNQNNYLAWYQQKPGKAPKLLIYWAQ | |
| | HLDSGVPSRFSGSGSGTDFTLTISSLQPEDFATYYCQQYYTYPYTFGCG | |
| | TKVEIK | |
| Humanized | QVQLQESGPGLVKPSQTLSLTCTVS | EIVLTQSPATLSLSPGERATLSCRA |
| mAb | GDSITSGYWNWIRQHPGKGLEYIG | SESVDNYGISFLNWFQQKPGQAPR |
| hCOH-1 | YISYSGSTYYNPSLKSRVTISRDTSK | LLIYAASNRATGIPARFSGSGSGTD |
| (City of | NQFSLKLSSVTAADTAVYYCASPN | FTLTISSLEPEDFAVYYCQQSKEVP |
| Hope) | YPFYAMDYWGQGTLVTVSS | WTFGGGTKVEIK (SEQ ID NO: 154) |
| (WO2017 | (SEQ ID NO: 153) | CDR1: ESVDNYGISF (non-Kabat) |
| 214170) | CDR1: GDSITSGY (non-Kabat) | (SEQ ID NO: 183) or |
| | (SEQ ID NO: 177) or SGYWN | RASESVDNYGISFLN |
| | (SEQ ID NO: 178) (Kabat) | (SEQ ID NO: 184) (Kabat) |
| | CDR2: ISYSGST (non-Kabat) | CDR2: AAS (SEQ ID NO: 185) |
| | (SEQ ID NO: 179) or | (non-Kabat) or AASNRAT |
| | YISYSGSTYYNPSLKS | (SEQ ID NO: 186) (Kabat) |
| | (SEQ ID NO: 180) (Kabat) | CDR3: QQSKEVPWT |
| | CDR3: ASPNYPFYAMDY (non- | (SEQ ID NO: 187) (Kabat) |
| | Kabat) (SEQ ID NO: 181) or | |
| | PNYPFYAMDY (SEQ ID NO: 182) | |
| | (Kabat) | |
| scFv | VL-VH (SEQ ID NO: 149) | |
| derived | EIVLTQSPATLSLSPGERATLSCRASESVDNYGISFLNWFQQKPGQAPRLLI | |
| from | YAASNRATGIPARFSGSGSGTDFTLTISSLEPEDFAVYYCQQSKEVPWTFGC | |
| hCOH-1 | GTKVEIKGGGGSGGGGSGGGGSGGGGSQVQLQESGPGLVKPSQTLSLTCTV | |
| (linker | SGDSITSGYWNWIRQHPGKCLEYIGYISYSGSTYYNPSLKSRVTISRDTS | |
| italicized) | KNQFSLKLSSVTAADTAVYYCASPNYPFYAMDYWGQGTLVTVSS | |
| | VH-VL (SEQ ID NO: 190) | |
| | QVQLQESGPGLVKPSQTLSLTCTVSGDSITSGYWNWIRQHPGKCLEYIGYI | |
| | SYSGSTYYNPSLKSRVTISRDTSKNQFSLKLSSVTAADTAVYYCASPNYPF | |
| | YAMDYWGQGTLVTVSSGGGGSGGGGSGGGGSGGGGSEIVLTQSPATLSLS | |
| | PGERATLSCRASESVDNYGISFLNWFQQKPGQAPRLLIYAASNRATGIPA | |
| | RFSGSGSGTDFTLTISSLEPEDFAVYYCQQSKEVPWTFGCGTKVEIK | |
| Humanized | EVQLQESGPGLVKPSQTLSLTCTVS | DIVLTQSPATLSLSPGERATLSCRA |
| mAb | GDSITSGYWNWIRQHPGKGLEYIG | SESVDNYGISFMNWFQQKPGQAP |
| hCOH-2 | YISYSGSTYYNPSLKSRVTISRDTSK | RLLIYAASNRATGIPARFSGSGSGT |
| (City of | NQYSLKLSSVTAADTAVYYCASPN | DFTLTISSLEPEDFAVYYCQQSKE |
| Hope) | YPFYAMDYWGQGTLVTVSS | VPWTFGGGTKVEIK |
| (WO2017 | (SEQ ID NO: 155) | (SEQ ID NO: 156) |
| 214170) | CDR1: GDSITSGY (non-Kabat) | CDR1: ESVDNYGISF (non-Kabat) |
| | (SEQ ID NO: 177) or SGYWN | (SEQ ID NO: 183) or |
| | (SEQ ID NO: 178) (Kabat) | RASESVDNYGISFMN |
| | CDR2: ISYSGST (non-Kabat) | (SEQ ID |
| | (SEQ ID NO: 179) or | NO: 188) (Kabat) |
| | YISYSGSTYYNPSLKS | CDR2: AAS (non-Kabat) |
| | (SEQ ID NO: 180) (Kabat) | (SEQ ID NO: 185) or AASNRAT |
| | CDR3: ASPNYPFYAMDY (non- | (SEQ ID NO: 186) (Kabat) |
| | Kabat) (SEQ ID NO: 181) or | CDR3: QQSKEVPWT |
| | PNYPFYAMDY (SEQ ID NO: 182) | (SEQ ID NO: 187) (Kabat) |
| | (Kabat) | |
| scFv | VL-VH (SEQ ID NO: 191) | |
| derived | DIVLTQSPATLSLSPGERATLSCRASESVDNYGISFMNWFQQKPGQAPRLL | |
| from | IYAASNRATGIPARFSGSGSGTDFTLTISSLEPEDFAVYYCQQSKEVPWTFG | |
| hCOH-2 | CGTKVEIKGGGGSGGGGSGGGGSGGGGSEVQLQESGPGLVKPSQTLSLTC | |
| (linker | TVSGDSITSGYWNWIRQHPGKCLEYIGYISYSGSTYYNPSLKSRVTISRDTS | |
| italicized) | KNQYSLKLSSVTAADTAVYYCASPNYPFYAMDYWGQGTLVTVSS | |
| | VH-VL (SEQ ID NO: 192) | |
| | EVQLQESGPGLVKPSQTLSLTCTVSGDSITSGYWNWIRQHPGKCLEYIGYI | |
| | SYSGSTYYNPSLKSRVTISRDTSKNQYSLKLSSVTAADTAVYYCASPNYPF | |
| | YAMDYWGQGTLVTVSSGGGGSGGGGSGGGGSGGGGSDIVLTQSPATLSL | |
| | SPGERATLSCRASESVDNYGISFMNWFQQKPGQAPRLLIYAASNRATGIPA | |
| | RFSGSGSGTDFTLTISSLEPEDFAVYYCQQSKEVPWTFGCGTKVEIK | |
| 1129_A01 | EVQLVQSGGGVVQPGRSLRLSCAA | EIVLTQSPSSLSASVGDRVTITCRA |
| (AB0369 | SGFTFSSYGMHWVRQAPGKCLEW | SQSISSYLNWYQQKPGKAPKLLIY |
| scFv) | VAVIWYDGSNKYYGDSVKGRFTIS | AASSLQSGVPSRFSGSGSGTDFTLT |
| | RDNSKNTLYLQMNSLRDEDTAVY | ISSLQPEDFATYYCQQSYSTPLTFG |
| | YCARRFTMLRGLIIEDYGMDVWGQ | CGTKVEIK (SEQ ID NO: 276) |
| | GTTVTVSS (SEQ ID NO: 310) | CDR1: RASQSISSYLN (SEQ ID |
| | CDR1: GFTFSSY (SEQ ID NO: 214) | NO: 217) |
| | CDR2: WYDGSN (SEQ ID NO: 215) | CDR2: AASSLQS (SEQ ID NO: 77) |
| | CDR3: RFTMLRGLIIEDYGMDV | CDR3: QQSYSTPLT (SEQ ID |
| | (SEQ ID NO: 216) | NO: 218) |
| 1203_A01 | CDR1: GFTFSSY (SEQ ID NO: 214) | CDR1: RASQSVSSNLA (SEQ ID |
| | CDR2: WYDGSN (SEQ ID NO: 215) | NO: 59) |
| | CDR3: RFTMLRGVFIEDYGMDV | CDR2: GASTRAT (SEQ ID NO: 60) |
| | (SEQ ID NO: 219) | CDR3: QQSYSTPLT (SEQ ID |
| | | NO: 218) |
| 1203_A02 | CDR1: GFTFSTY (SEQ ID NO: 220) | CDR1: RASQSISSYLN (SEQ ID |
| | CDR2: WYDGSN (SEQ ID NO: 215) | NO: 217) |
| | CDR3: RNTMVRGVIIEDYGMDV | CDR2: AASSLQS (SEQ ID NO: 77) |
| | (SEQ ID NO: 221) | CDR3: QQSYSSPLT (SEQ ID |
| | | NO: 222) |
| AB0605s | EVQLVQSGGGVVQPGRSLRLSCAA | EIVLTQSPSSLSASVGDRVTITCRA |
| cfv | SGFTFSSYGMHWVRQAPGKCLEW | SQSISSYLNWYQQKPGKAPKLLIY |
| | VAVIWYDGSNKYYGDSVKGRFTIS | AASSLQSGVPSRFSGSGSGTDFTLT |
| | RDNSKNTLYLQMNSLRDEDTAVY | ISSLQPEDFATYYCQQSYSTPLTFG |
| | YCARRFTMLRGQYIEDYGMDVWG | CGTKVEIK (SEQ ID NO: 276) |
| | QGTTVTVSS (SEQ ID NO: 277) | CDR1: RASQSISSYLN (SEQ ID |
| | CDR1: GFTFSSY (SEQ ID NO: 214) | NO: 217) |
| | CDR2: WYDGSN (SEQ ID NO: 215) | CDR2: AASSLQS (SEQ ID NO: 77) |
| | CDR3: RFTMLRGQYIEDYGMDV | CDR3: QQSYSTPLT (SEQ ID |
| | (SEQ ID NO: 223) | NO: 218) |
| AB0606s | EVQLVQSGGGVVQPGRSLRLSCAA | EIVLTQSPSSLSASVGDRVTITCRA |
| cfv | SGFTFSSYGMHWVRQAPGKCLEW | SQSISSYLNWYQQKPGKAPKLLIY |
| | VAVIWYDGSNKYYGDSVKGRFTIS | AASSLQSGVPSRFSGSGSGTDFTLT |
| | RDNSKNTLYLQMNSLRDEDTAVY | ISSLQPEDFATYYCQQSYSTPLTFG |
| | YCARRFTMLRGWYIEDYGMDVWG | CGTKVEIK (SEQ ID NO: 276) |
| | QGTTVTVSS (SEQ ID NO: 278) | CDR1: RASQSISSYLN (SEQ ID |
| | CDR1: GFTFSSY (SEQ ID NO: 214) | NO: 217) |
| | CDR2: WYDGSN (SEQ ID NO: 215) | CDR2: AASSLQS (SEQ ID NO: 77) |
| | CDR3: RFTMLRGWYIEDYGMDV | CDR3: QQSYSTPLT (SEQ ID |
| | (SEQ ID NO: 224) | NO: 218) |
| AB0622s | EVQLVQSGGGVVQPGRSLRLSCAA | EIVLTQSPSSLSASVGDRVTITCRA |
| cfv | SGFTFSSYGMHWVRQAPGKCLEW | SQSISSYLNWYQQKPGKAPKLLIY |
| | VAVIWYDGSNKYYGDSVKGRFTIS | AASSLQSGVPSRFSGSGSGTDFTLT |
| | RDNSKNTLYLQMNSLRDEDTAVY | ISSLQPEDFATYYCQQSYSTPLTFG |
| | YCARRFTMLRGWIIEDYGMDVWG | CGTKVEIK (SEQ ID NO: 276) |
| | QGTTVTVSS (SEQ ID NO: 279) | CDR1: RASQSISSYLN (SEQ ID |
| | CDR1: GFTFSSY (SEQ ID NO: 214) | NO: 217) |
| | CDR2: WYDGSN (SEQ ID NO: 215) | CDR2: AASSLQS (SEQ ID NO: 77) |
| | CDR3: RFTMLRGWIIEDYGMDV | CDR3: QQSYSTPLT (SEQ ID |
| | (SEQ ID NO: 225) | NO: 218) |
| Consensus | CDR1: GFTFSSY (SEQ ID NO: 214) | CDR1: RASQSISSYLN (SEQ ID |
| 1 | CDR2: WYDGSN (SEQ ID NO: 215) | NO: 217) |
| (AB0605, | CDR3: RFTMLRGX₁X₂IEDYGMDV | CDR2: AASSLQS (SEQ ID NO: 77) |
| AB0606, | where X₁ is Q or W, and X₂ is I or Y | CDR3: QQSYSTPLT (SEQ ID |
| AB0622) | (SEQ ID NO: 226) | NO: 218) |
| AB0679s | EVQLVQSGGGVVQPGRSLRLSCAA | EIVLTQSPSSLSASVGDRVTITCRA |
| cFv | SGFPFSSYGMHWVRQAPGKCLEW | SQSISSYLNWYQQKPGKAPKLLIY |
| | VAVIWYDGSNKYYGDSVKGRFTIS | AASSLQSGVPSRFSGSGSGTDFTLT |
| | RDNSKNTLYLQMNSLRDEDTAVY | ISSLQPEDFATYYCQQSYSTPLTFG |
| | YCARRFTMLRGWYIEDYGMDVWG | CGTKVEIK (SEQ ID NO: 276) |
| | QGTTVTVSS (SEQ ID NO: 280) | CDR1: RASQSISSYLN (SEQ ID |
| | CDR1: GFPFSSY (SEQ ID NO: 227) | NO: 217) |
| | CDR2: WYDGSN (SEQ ID NO: 215) | CDR2: AASSLQS (SEQ ID NO: 77) |
| | CDR3: RFTMLRGWYIEDYGMDV | CDR3: QQSYSTPLT (SEQ ID |
| | (SEQ ID NO: 224) | NO: 218) |
| AB0681s | EVQLVQSGGGVVQPGRSLRLSCAA | EIVLTQSPSSLSASVGDRVTITCRA |
| cFv | SGEWFSSYGMHWVRQAPGKCLEW | SQSISSYLNWYQQKPGKAPKLLIY |
| | VAVIWYDGSNKYYGDSVKGRFTIS | AASSLQSGVPSRFSGSGSGTDFTLT |
| | RDNSKNTLYLQMNSLRDEDTAVY | ISSLQPEDFATYYCQQSYSTPLTFG |
| | YCARRFTHLRGWIIEDYGMDVWG | CGTKVEIK (SEQ ID NO: 276) |
| | QGTTVTVSS (SEQ ID NO: 281) | CDR1: RASQSISSYLN (SEQ ID |
| | CDR1: GEWESSY (SEQ ID NO: 228) | NO: 217) |
| | CDR2: WYDGSN (SEQ ID NO: 215) | CDR2: AASSLQS (SEQ ID NO: 77) |
| | CDR3: RFTHLRGWIIEDYGMDV | CDR3: QQSYSTPLT (SEQ ID |
| | (SEQ ID NO: 229) | NO: 218) |
| AB0682s | EVQLVQSGGGVVQPGRSLRLSCAA | EIVLTQSPSSLSASVGDRVTITCRA |
| cFv | SGFTFSSSGMHWVRQAPGKCLEWV | SQSISSYLNWYQQKPGKAPKLLIY |
| | AVIWYDGSNKYYGDSVKGRFTISR | AASSLQSGVPSRFSGSGSGTDFTLT |
| | DNSKNTLYLQMNSLRDEDTAVYY | ISSLQPEDFATYYCQQSYSTPLTFG |
| | CARRFTMLRGWYIEDYGMDVWGQ | CGTKVEIK (SEQ ID NO: 276) |
| | GTTVTVSS (SEQ ID NO: 282) | CDR1: RASQSISSYLN (SEQ ID |
| | CDR1: GFTFSSS (SEQ ID NO: 230) | NO: 217) |
| | CDR2: WYDGSN (SEQ ID NO: 215) | CDR2: AASSLQS (SEQ ID NO: 77) |
| | CDR3: RFTMLRGWYIEDYGMDV | CDR3: QQSYSTPLT (SEQ ID |
| | (SEQ ID NO: 224) | NO: 218) |
| Consensus | CDR1: GX₁X₂FSSX₃ where X₁ is For | CDR1: RASQSISSYLN (SEQ ID |
| 2 | E, X₂ T, P, or W, X₃ is Y or S | NO: 217) |
| (AB0679, | (SEQ ID NO: 231) | CDR2: AASSLQS (SEQ ID NO: 77) |
| AB0681, | CDR2: WYDGSN (SEQ ID NO: 215) | CDR3: QQSYSTPLT (SEQ ID |
| AB0682) | CDR3: RFTX₁LRGWX₂IEDYGMDV | NO: 218) |
| | where X₁ is M or H, X₂ is I or Y | |
| | (SEQ ID NO: 232) | |
| AB0898 | EVQLVQSGGGVVQPGRSLRLSCAA | EIVLTQSPSSLSASVGDRVTITCRA |
| | SGFTFSSSGMHWVRQAPGKCLEWV | SQSISSYLNWYQQKPGKAPKLLIY |
| | AVIWYDASNKYYGDSVKGRFTISR | AASSLQSGVPSRFSGSGSGTDFTLT |
| | DNSKNTLYLQMNSLRDEDTAVYY | ISSLQPEDFATYYCQQSYSTPLTFG |
| | CARRFTRLRGWYIEDYGLDVWGQ | CGTKVEIK (SEQ ID NO: 276) |
| | GTTVTVSS (SEQ ID NO: 283) | CDR1: RASQSISSYLN (SEQ ID |
| | CDR1: GFTFSSS (SEQ ID NO: 230) | NO: 217) |
| | CDR2: WYDASN (SEQ ID NO: 233) | CDR2: AASSLQS (SEQ ID NO: 77) |
| | CDR3: RFTRLRGWYIEDYGLDV | CDR3: QQSYSTPLT (SEQ ID |
| | (SEQ ID NO: 242) | NO: 218) |
| AB0899 | EVQLVQSGGGVVQPGRSLRLSCAA | EIVLTQSPSSLSASVGDRVTITCRA |
| | SGFTFSSSGMHWVRQAPGKCLEWV | SQSISSYLNWYQQKPGKAPKLLIY |
| | AVIWYDASNKYYGDSVKGRFTISR | AASSLQSGVPSRFSGSGSGTDFTLT |
| | DNSKNTLYLQMNSLRDEDTAVYY | ISSLQPEDFATYYCQQSYSTPLTFG |
| | CARRFTYLRGWYIEDYGLDVWGQ | CGTKVEIK (SEQ ID NO: 276) |
| | GTTVTVSS (SEQ ID NO: 284) | CDR1: RASQSISSYLN (SEQ ID |
| | CDR1: GFTFSSS (SEQ ID NO: 230) | NO: 217) |
| | CDR2: WYDASN (SEQ ID NO: 233) | CDR2: AASSLQS (SEQ ID NO: 77) |
| | CDR3: RFTYLRGWYIEDYGLDV | CDR3: QQSYSTPLT (SEQ ID |
| | (SEQ ID NO: 234) | NO: 218) |
| AB0900 | EVQLVQSGGGVVQPGRSLRLSCAA | EIVLTQSPSSLSASVGDRVTITCRA |
| | SGFTFSSSGMHWVRQAPGKCLEWV | SQSISSYLNWYQQKPGKAPKLLIY |
| | AVIWYDASNKYYGDSVKGRFTISR | AASSLQSGVPSRFSGSGSGTDFTLT |
| | DNSKNTLYLQMNSLRDEDTAVYY | ISSLQPEDFATYYCQQSYSTPLTFG |
| | CARRFTSLRGWYIEDYGLDVWGQ | CGTKVEIK (SEQ ID NO: 276) |
| | GTTVTVSS (SEQ ID NO: 285) | CDR1: RASQSISSYLN (SEQ ID |
| | CDR1: GFTFSSS (SEQ ID NO: 230) | NO: 217) |
| | CDR2: WYDASN (SEQ ID NO: 233) | CDR2: AASSLQS (SEQ ID NO: 77) |
| | CDR3: RFTSLRGWYIEDYGLDV | CDR3: QQSYSTPLT (SEQ ID |
| | (SEQ ID NO: 235) | NO: 218) |
| Consensus | CDR1: GFTFSSS (SEQ ID NO: 230) | CDR1: RASQSISSYLN (SEQ ID |
| 3 | CDR2: WYDASN (SEQ ID NO: 233) | NO: 217) |
| (AB0898, | CDR3: RFTXLRGWYIEDYGLDV | CDR2: AASSLQS (SEQ ID NO: 77) |
| AB0899, | where X is R, Y, or S | CDR3: QQSYSTPLT (SEQ ID |
| AB0900) | (SEQ ID NO: 236) | NO: 218) |
| AB1080s | EVQLVQSGGGVVQPGRSLRLSCAA | EIVLTQSPSSLSASVGDRVTITCRA |
| cFv | SGFTFSSYGMHWVRQAPGKCLEW | SQSISSYLNWYQQKPGKAPKLLIY |
| | VAVIWYDASNKYYGDSVKGRFTIS | AASSLQSGVPSRFSGSGSGTDFTLT |
| | RDNSKNTLYLQMNSLRDEDTAVY | ISSLQPEDFATYYCQQSYSIPLTFG |
| | YCARRFTHLRGWYIEDYGLDVWG | CGTKVEIK (SEQ ID NO: 253) |
| | QGTTVTVSS (SEQ ID NO: 286) | CDR1: RASQSISSYLN (SEQ ID |
| | CDR1: GFTFSSY (SEQ ID NO: 214) | NO: 217) |
| | CDR2: WYDASN (SEQ ID NO: 233) | CDR2: AASSLQS (SEQ ID NO: 77) |
| | CDR3: RFTHLRGWYIEDYGLDV | CDR3: QQSYSIPLT (SEQ ID |
| | (SEQ ID NO: 237) | NO: 249) |
| AB1081s | EVQLVQSGGGVVQPGRSLRLSCAA | EIVLTQSPSSLSASVGDRVTITCRA |
| cFv | SGFAFSSYGMHWVRQAPGKCLEW | SQSISSYLNWYQQKPGKAPKLLIY |
| | VAVIWYDESNKYYGDSVKGRFTIS | AASSLQSGVPSRFSGSGSGTDFTLT |
| | RDNSRNTLYLQMNSLRDEDTAVYY | ISSLQPEDFATYYCQQSYSIPLTFG |
| | CARRFTNLRGWIIEDYGLDVWGQG | CGTKVEIK (SEQ ID NO: 253) |
| | TTVTVSS (SEQ ID NO: 287) | CDR1: RASQSISSYLN (SEQ ID |
| | CDR1: GFAFSSY (SEQ ID NO: 238) | NO: 217) |
| | CDR2: WYDESN (SEQ ID NO: 239) | CDR2: AASSLQS (SEQ ID NO: 77) |
| | CDR3: RFTNLRGWIIEDYGLDV | CDR3: QQSYSIPLT (SEQ ID |
| | (SEQ ID NO: 240) | NO: 249) |
| AB1084s | EVQLVQSGGGVVQPGRSLRLSCAA | EIVLTQSPSSLSASVGDRVTITCRA |
| cFv | SGFTFSMYGMHWVRQAPGKCLEW | SQSISSYLNWYQQKPGKAPKLLIY |
| | VAVIWYDASNKYYGDSVKGRFTIS | AASSLQSGVPSRFSGSGSGTDFTLT |
| | RDNSKNTLYLQMNSLRDEDTAVY | ISSLQPEDFATYYCQQSYSIPLTFG |
| | YCARRFTRLRGWYIEDYGLDVWG | CGTKVEIK (SEQ ID NO: 253) |
| | QGTTVTVSS (SEQ ID NO: 288) | CDR1: RASQSISSYLN (SEQ ID |
| | CDR1: GFTFSMY (SEQ ID NO: 241) | NO: 217) |
| | CDR2: WYDASN (SEQ ID NO: 233) | CDR2: AASSLQS (SEQ ID NO: 77) |
| | CDR3: RFTRLRGWYIEDYGLDV | CDR3: QQSYSIPLT (SEQ ID |
| | (SEQ ID NO: 242) | NO: 249) |
| AB1085s | EVQLVQSGGGVVQPGRSLRLSCAA | EIVLTQSPSSLSASVGDRVTITCRA |
| cFv | SGFTFGSYGMHWVRQAPGKCLEW | SQSISSYLNWYQQKPGKAPKLLIY |
| | VAVIWYDGSNKYYGDSVKGRFTIS | AASSLQSGVPSRFSGSGSGTDFTLT |
| | RDNSKNTLYLQMNSLRDEDTAVY | ISSLQPEDFATYYCQQSYSIPLTFG |
| | YCARRFTHLRGQYIEDYGMDVWG | CGTKVEIK (SEQ ID NO: 253) |
| | QGTTVTVSS (SEQ ID NO: 289) | CDR1: RASQSISSYLN (SEQ ID |
| | CDR1: GFTFGSY (SEQ ID NO: 243) | NO: 217) |
| | CDR2: WYDGSN (SEQ ID NO: 215) | CDR2: AASSLQS (SEQ ID NO: 5) |
| | CDR3: RFTHLRGQYIEDYGMDV | CDR3: QQSYSIPLT (SEQ ID |
| | (SEQ ID NO: 244) | NO: 249) |
| Consensus | CDR1: GFX₁FX₂X₃Y where X₁ is T or | CDR1: RASQSISSYLN (SEQ ID |
| 4 | A, X₂ is S or G, X₃ is S or M (SEQ ID | NO: 217) |
| (AB1080, | NO: 245) | CDR2: AASSLQS (SEQ ID NO: 77) |
| AB1081, | CDR2: WYDXSN where X is G, A, or | CDR3: QQSYSXPLT where X is T or |
| AB1084, | E (SEQ ID NO: 246) | I (SEQ ID NO: 259) |
| AB1085) | CDR3: RFTX₁LRGX₂X₃IEDYGX₄DV | |
| | where X₁ is H, N, or R, X₂ is W or Q, | |
| | X₃ is I or Y, X₄ is M or L (SEQ ID | |
| | NO: 247) | |
| AB1424/ | EVQLVQSGGGVVQPGRSLRLSCAA | EIVLTQSPSSLSASVGDRVTITCRA |
| AB1612 | SGFTFSSYGMHWVRQAPGKGLEW | SQSISSYLNWYQQKPGKAPKLLIY |
| | VAVIWYDASNKYYGDSVKGRFTIS | AASSLQSGVPSRFSGSGSGTDFTL |
| | RDNSKNTLYLQMNSLRDEDTAVY | TISSLQPEDFATYYCQQSYSIPLTF |
| | YCARRFTHLRGQYIEDYGLDVWG | GGGTKVEIK (SEQ ID NO: 251) |
| | QGTTVTVSS (SEQ ID NO: 250) | CDR1: RASQSISSYLN (SEQ ID |
| | CDR1: GFTFSSY (SEQ ID NO: 214) | NO: 217) |
| | CDR2: WYDASN (SEQ ID NO: 233) | CDR2: AASSLQS (SEQ ID NO: 77) |
| | CDR3: RFTHLRGQYIEDYGLDV | CDR3: QQSYSIPLT (SEQ ID |
| | (SEQ ID NO: 248) | NO: 249) |
| AB1424/ | EVQLVQSGGGVVQPGRSLRLSCAA | EIVLTQSPSSLSASVGDRVTITCRA |
| 1612 | SGFTFSSYGMHWVRQAPGKCLEW | SQSISSYLNWYQQKPGKAPKLLIY |
| (with | VAVIWYDASNKYYGDSVKGRFTIS | AASSLQSGVPSRFSGSGSGTDFTL |
| cysteine | RDNSKNTLYLQMNSLRDEDTAVY | TISSLQPEDFATYYCQQSYSIPLTF |
| hetero- | YCARRFTHLRGQYIEDYGLDVWG | GCGTKVEIK (SEQ ID NO: 253) |
| dimerization | QGTTVTVSS (SEQ ID NO: 252) | CDR1: RASQSISSYLN (SEQ ID |
| mutations | CDR1: GFTFSSY (SEQ ID NO: 214) | NO: 217) |
| for | CDR2: WYDASN (SEQ ID NO: 233) | CDR2: AASSLQS (SEQ ID NO: 77) |
| disulfide | CDR3: RFTHLRGQYIEDYGLDV | CDR3: QQSYSIPLT (SEQ ID |
| bridge | (SEQ ID NO: 248) | NO: 249) |
| formation) | | |
| AB1424/ | EVQLVQSGGGVVQPGRSLRLSCAASGFTFSSYGMHWVRQAPGKCLEWV | |
| 1612 | AVIWYDASNKYYGDSVKGRFTISRDNSKNTLYLQMNSLRDEDTAVYYCA | |
| scFv | RRFTHLRGQYIEDYGLDVWGQGTTVTVSSGGGGSGGGGSGGGGSGGGGS | |
| (VH-VL) | EIVLTQSPSSLSASVGDRVTITCRASQSISSYLNWYQQKPGKAPKLLIY | |
| | AASSLQSGVPSRFSGSGSGTDFTLTISSLQPEDFATYYCQQSYSIPLTF | |
| | GCGTKVEIK | |
| | (SEQ ID NO: 254) | |
| AB | EIVLTQSPSSLSASVGDRVTITCRASQSISSYLNWYQQKPGKAPKLLIYA | |
| 1424/161 | ASSLQSGVPSRFSGSGSGTDFTLTISSLQPEDFATYYCQQSYSIPLTFGC | |
| 2 scFv | GTKVEIKGGGGSGGGGSGGGGSGGGGSEVQLVQSGGGVVQPGRSLRLSCA | |
| (VL-VH) | ASGFTFSSYGMHWVRQAPGKCLEWVAVIWYDASNKYYGDSVKGRFTISRD | |
| | NSKNTLYLQMNSLRDEDTAVYYCARRFTHLRGQYIEDYGLDVWGQGT | |
| | TVTVSS | |
| | (SEQ ID NO: 255) | |
| Consensus | CDR1: GFTFXSY where X is S or G | CDR1: RASQSISSYLN (SEQ ID |
| 5 | (SEQ ID NO: 256) | NO: 217) |
| (AB0369, | CDR2: WYDXSN where X is G or A | CDR2: AASSLQS (SEQ ID NO: 77) |
| AB1080, | (SEQ ID NO: 257) | CDR3: QQSYSXPLT where X is T or |
| AB1085, | CDR3: RFTX₁LRGX₂X₃IEDYGX₄DV | I (SEQ ID NO: 259) |
| AB1424/ | where X₁ is M or H, X₂ is L, W, or Q, | |
| AB1612) | X₃ is I or Y, X₄ is M or L (SEQ ID | |
| | NO: 258) | |
| Consensus | CDR1: GX₁X₂FX₃X₄X₅ where X₁ is For | CDR1: RASQSISSYLN (SEQ ID |
| 6 | E, X₂ is T, P, W, or A, X₃ is S or G, X₄ | NO: 217) |
| (master | is S or M, X₅ is Y or S (SEQ ID | CDR2: AASSLQS (SEQ ID NO: 77) |
| consensus) | NO: 260) | CDR3: QQSYSXPLT where X is T or |
| | CDR2: WYDXSN where X is G, A, or | I (SEQ ID NO: 259) |
| | E (SEQ ID NO: 249) | |
| | CDR3: RFTX₁LRGX₂X₃IEDYGX₄DV | |
| | where X₁ is M, H, N, R, Y, or S, X₂ is L, | |
| | Q, or W, X₃ is I or Y, X₄ is M or L | |
| | (SEQ ID NO: 261) | |
| 3A1 | QVQLQQPGAELVKPGASVKLSCKA | DVVMTQTPLSLPVSLGDQASISCR |
| | SGYTFTSYWMHWVKQRPGQGLE | SSQSIVHSNGNTYLEWYLQKPGQ |
| | WIGEIDPFDSYTNYNQNFKGKATL | SPKLLIYKVSNRLSGVPDRFSGSG |
| | TVDKSSSTAYMLLSSLTSDDSAVY | SGTDFTLKISRVEAEDLGVYYCFQ |
| | YCARERLRLWSYYFDYWGQGTTL | GSHDPFTFGSGTKLEIK (SEQ ID |
| | TVSS (SEQ ID NO: 263) | NO: 264) |
| | CDR1: GYTFTSY (SEQ ID NO: 291) | CDR1: RSSQSIVHSNGNTYLE (SEQ |
| | CDR2: DPFDSY (SEQ ID NO: 292) | ID NO: 294) |
| | CDR3: ERLRLWSYYFDY (SEQ ID | CDR2: KVSNRLS (SEQ ID NO: 295) |
| | NO: 293) | CDR3: FQGSHDPFT (SEQ ID |
| | | NO: 296) |
| 7G4 | QVQLQQPGAELVKPGASVKLSCKA | DVVMTQTPLSLPVSLGDQASISCR |
| | SGYTFTSYWMHWVKQRPGQGLE | SSQSIVHSNGNTYLEWYLQKPGQ |
| | WIGEVDPSDSYTNYNQKFKGKATL | SPKLLIYKVSNRLSGVPDRESGSG |
| | TVDKSSSTAYILLSNLTSDDSAVYY | SGTDFTLKISRVEAEDLGVYYCFQ |
| | CARERVRLWSYFFDYWGQGTTLT | GSHDPFTFGSGTKLEIK (SEQ ID |
| | VSS (SEQ ID NO: 265) | NO: 266) |
| | CDR1: GYTFTSY (SEQ ID NO: 291) | CDR1: RSSQSIVHSNGNTYLE (SEQ |
| | CDR2: DPSDSY (SEQ ID NO: 297) | ID NO: 294) |
| | CDR3: ERVRLWSYFFDY (SEQ ID | CDR2: KVSNRLS (SEQ ID NO: 295) |
| | NO: 298) | CDR3: FQGSHDPFT (SEQ ID |
| | | NO: 296) |
| 1B3-A7 | EVQLVESGGGLVKPGGSLKLSCVV | DVVMTQTPLSLPVSPGDQASISCR |
| | SGFTFSNYAMSWVRQTPEKRLEW | SSQSLVHSNGNTYLYWYLQKPG |
| | VATISDGGGYTYYPDSVKGRFTISR | QSPKLLIYRVSNRFSGVPDRESGS |
| | DNAKNNLYLQMSHLKSEDTAIYYC | GSGTDFTLKINRVEAEDLGVYFCF |
| | ARDDLGGGNYVSSYFDVWGTGTT | QGTHVPLTFGSGTKLELK (SEQ |
| | VTVSS (SEQ ID NO: 267) | ID NO: 268) |
| | CDR1: GFTFSNY (SEQ ID NO: 299) | CDR1: RSSQSLVHSNGNTYLY |
| | CDR2: DGGGY (SEQ ID NO: 300) | (SEQ ID NO: 302) |
| | CDR3: DDLGGGNYVSSYFDV (SEQ | CDR2: RVSNRFS (SEQ ID NO: 303) |
| | ID NO: 301) | CDR3: FQGTHVPLT (SEQ ID |
| | | NO: 304) |
| 10H7-C5 | EVQLVESGGGLVKPGGSLKLSCAA | DIVMTQTPLSLPVSLGDQASISCRS |
| | SGFSFSRYAMSWVRQTPEKRLEW | SQSLLHSNGNTYLYWYLQKPGQ |
| | VATISDGGSYTHYRDNVKGRFTISR | SPKLLIHRVSNRFSGVPDRFGGSG |
| | DNAKNNLNLQMSHLKSEDTAIYYC | SGTDFTLKIIRVEAEDLGVYFCFQ |
| | ARNEMGLYFDYDVYAMDYWGQG | GTHVPWTFGGGTKLEIK (SEQ ID |
| | TSVTVSS (SEQ ID NO: 269) | NO: 262) |
| | CDR1: GFSFSRY (SEQ ID NO: 305) | CDR1: RSSQSLLHSNGNTYLY |
| | CDR2: DGGSY (SEQ ID NO: 306) | (SEQ ID NO: 308) |
| | CDR3: NEMGLYFDYDVYAMDY | CDR2: RVSNRFS (SEQ ID NO: 303) |
| | (SEQ ID NO: 307) | CDR3: FQGTHVPWT (SEQ ID |
| | | NO: 309) |

In certain embodiments, the second antigen-binding site that binds BAFF-R (e.g., human BAFF-R) comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 2, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in Table 2. In certain embodiments, the second antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), or any other CDR determination method known in the art, of the VH and VL sequences of an antigen-binding site disclosed in Table 2. In certain embodiments, the second antigen-binding site comprises the heavy chain CDR1, CDR2, and CDR3 and the light chain CDR1, CDR2, and CDR3 of an antibody disclosed in Table 2.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:145, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:146. In certain embodiments, the VH comprises CDR1, CDR2, and CDR3 comprising the amino acid sequences of SEQ ID NOs: 157 or 135, 158 or 136, and 159 or 137, respectively (e.g., SEQ ID NOs: 157, 158, and 159, respectively; or SEQ ID NOs: 135, 136, and 137, respectively). In certain embodiments, the VL comprises CDR1, CDR2, and CDR3 comprising the amino acid sequences of SEQ ID NOs: 160, 161, and 162, respectively. In certain embodiments, the second antigen-binding site comprises (a) a VH that comprises CDR1, CDR2, and CDR3 comprising the amino acid sequences of SEQ ID NOs: 157 or 135, 158 or 136, and 159 or 137, respectively (e.g., SEQ ID NOs: 157, 158, and 159, respectively; or SEQ ID NOs: 135, 136, and 137, respectively); and (b) a VL that comprises CDR1, CDR2, and CDR3 comprising the amino acid sequences of SEQ ID NOs: 160, 161, and 162, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 207 or 138.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:147, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:148. In certain embodiments, the VH comprises CDR1, CDR2, and CDR3 comprising the amino acid sequences of SEQ ID NOs: 163, 164, and 165, respectively. In certain embodiments, the VL comprises CDR1, CDR2, and CDR3 comprising the amino acid sequences of SEQ ID NOs: 166, 167, and 168, respectively. In certain embodiments, the second antigen-binding site comprises (a) a VH that comprises CDR1, CDR2, and CDR3 comprising the amino acid sequences of SEQ ID NOs: 163, 164, and 165, respectively; and (b) a VL that comprises CDR1, CDR2, and CDR3 comprising the amino acid sequences of SEQ ID NOs:166, 167, and 168, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 139 or 140.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:147, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:150. In certain embodiments, the VH comprises CDR1, CDR2, and CDR3 comprising the amino acid sequences of SEQ ID NOs: 163, 164, and 165, respectively. In certain embodiments, the VL comprises CDR1, CDR2, and CDR3 comprising the amino acid sequences of SEQ ID NOs: 169, 170, and 168, respectively. In certain embodiments, the second antigen-binding site comprises (a) a VH that comprises CDR1, CDR2, and CDR3 comprising the amino acid sequences of SEQ ID NOs: 163, 164, and 165, respectively; and (b) a VL that comprises CDR1, CDR2, and CDR3 comprising the amino acid sequences of SEQ ID NOs: 169, 170, and 168, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 141 or 142.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:151, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:152. In certain embodiments, the VH comprises CDR1, CDR2, and CDR3 comprising the amino acid sequences of SEQ ID NOs: 171, 172, and 173, respectively. In certain embodiments, the VL comprises CDR1, CDR2, and CDR3 comprising the amino acid sequences of SEQ ID NOs: 174, 175, and 176, respectively. In certain embodiments, the second antigen-binding site comprises (a) a VH that comprises CDR1, CDR2, and CDR3 comprising the amino acid sequences of SEQ ID NOs: 171, 172, and 173, respectively; and (b) a VL that comprises CDR1, CDR2, and CDR3 comprising the amino acid sequences of SEQ ID NOs: 174, 175, and 176, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 143 or 144.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:153, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:154. In certain embodiments, the VH comprises CDR1, CDR2, and CDR3 comprising the amino acid sequences of SEQ ID NOs: 177 or 178, 179 or 180, and 181 or 182, respectively (e.g., SEQ ID NOs: 177, 179, and 181, respectively; or SEQ ID NOs: 178, 180, and 182, respectively). In certain embodiments, the VL comprises CDR1, CDR2, and CDR3 comprising the amino acid sequences of SEQ ID NOs: 183 or 184, 185 or 186, and 187, respectively (e.g., SEQ ID NOs: 183, 185, and 187, respectively; or SEQ ID NOs: 184, 186, and 187, respectively). In certain embodiments, the second antigen-binding site comprises (a) a VH that comprises CDR1, CDR2, and CDR3 comprising the amino acid sequences of SEQ ID NOs: 177 or 178, 179 or 180, and 181 or 182, respectively (e.g., SEQ ID NOs: 177, 179, and 181, respectively; or SEQ ID NOs: 178, 180, and 182, respectively); and (b) a VL that comprises CDR1, CDR2, and CDR3 comprising the amino acid sequences of SEQ ID NOs: 183 or 184, 185 or 186, and 187, respectively (e.g., SEQ ID NOs: 183, 185, and 187, respectively; or SEQ ID NOs: 184, 186, and 187, respectively). In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 149 or 190.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:155, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:156. In certain embodiments, the VH comprises CDR1, CDR2, and CDR3 comprising the amino acid sequences of SEQ ID NOs: 177 or 178, 179 or 180, and 181 or 182, respectively (e.g., SEQ ID NOs: 177, 179, and 181, respectively; or SEQ ID NOs: 178, 180, and 182, respectively). In certain embodiments, the VL comprises CDR1, CDR2, and CDR3 comprising the amino acid sequences of SEQ ID NOs: 183 or 188, 185 or 186, and 187, respectively (e.g., SEQ ID NOs: 183, 185, and 187, respectively; or SEQ ID NOs: 188, 186, and 187, respectively). In certain embodiments, the second antigen-binding site comprises (a) a VH that comprises CDR1, CDR2, and CDR3 comprising the amino acid sequences of SEQ ID NOs: 177 or 178, 179 or 180, and 181 or 182, respectively (e.g., SEQ ID NOs: 177, 179, and 181, respectively; or SEQ ID NOs: 178, 180, and 182, respectively); and (b) a VL that comprises CDR1, CDR2, and CDR3 comprising the amino acid sequences of SEQ ID NOs: 183 or 188, 185 or 186, and 187, respectively (e.g., SEQ ID NOs: 183, 185, and 187, respectively; or SEQ ID NOs: 188, 186, and 187, respectively). In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 191 or 192.

In certain embodiments, the VH of the second antigen-binding site that binds BAFF-R comprises CDR1, CDR2, and CDR3 comprising the amino acid sequences of SEQ ID NOs: 260, 249, and 261, respectively. In certain embodiments, the VL of the second antigen-binding site that binds BAFF-R comprises CDR1, CDR2, and CDR3 comprising the amino acid sequences of SEQ ID NOs: 217, 77, and 259, respectively. In certain embodiments, the second antigen-binding site comprises (a) a VH that comprises CDR1, CDR2, and CDR3 comprising the amino acid sequences of SEQ ID NOs: 260, 249 and 261, respectively; and (b) a VL that comprises CDR1, CDR2, and CDR3 comprising the amino acid sequences of SEQ ID NOs: 217, 77, and 259, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:310, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:276. In certain embodiments, the VH comprises CDR1, CDR2, and CDR3 comprising the amino acid sequences of SEQ ID NOs: 214, 215, and 216, respectively. In certain embodiments, the VL comprises CDR1, CDR2, and CDR3 comprising the amino acid sequences of SEQ ID NOs: 217, 77, and 218, respectively. In certain embodiments, the second antigen-binding site comprises (a) a VH that comprises CDR1, CDR2, and CDR3 comprising the amino acid sequences of SEQ ID NOs: 214, 215, and 216, respectively; and (b) a VL that comprises CDR1, CDR2, and CDR3 comprising the amino acid sequences of SEQ ID NOs: 217, 77, and 218, respectively.

In certain embodiments, the VL comprises CDR1, CDR2, and CDR3 comprising the amino acid sequences of SEQ ID NOs: 59, 60, and 218, respectively. In certain embodiments, the second antigen-binding site that binds BAFF-R comprises (a) a VH that comprises CDR1, CDR2, and CDR3 comprising the amino acid sequences of SEQ ID NOs: 214, 215, and 219, respectively; and (b) a VL that comprises CDR1, CDR2, and CDR3 comprising the amino acid sequences of SEQ ID NOs: 59, 60, and 218, respectively.

In certain embodiments, the VH comprises CDR1, CDR2, and CDR3 comprising the amino acid sequences of SEQ ID NOs: 220, 215, and 221, respectively. In certain embodiments, the VL comprises CDR1, CDR2, and CDR3 comprising the amino acid sequences of SEQ ID NOs: 217, 77, and 222, respectively. In certain embodiments, the second antigen-binding site that binds BAFF-R comprises (a) a VH that comprises CDR1, CDR2, and CDR3 comprising the amino acid sequences of SEQ ID NOs: 220, 215, and 221, respectively; and (b) a VL that comprises CDR1, CDR2, and CDR3 comprising the amino acid sequences of SEQ ID NOs: 217, 77, and 222, respectively.

In certain embodiments, the VH of the second antigen-binding site that binds BAFF-R comprises CDR1, CDR2, and CDR3 comprising the amino acid sequences of SEQ ID NOs: 214, 215, and 226, respectively. In certain embodiments, the VL of the second antigen-binding site that binds BAFF-R comprises CDR1, CDR2, and CDR3 comprising the amino acid sequences of SEQ ID NOs:217, 77, and 218, respectively. In certain embodiments, the second antigen-binding site comprises (a) a VH that comprises CDR1, CDR2, and CDR3 comprising the amino acid sequences of SEQ ID NOs: 214, 215, and 226, respectively; and (b) a VL that comprises CDR1, CDR2, and CDR3 comprising the amino acid sequences of SEQ ID NOs: 217, 77, and 218, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:277, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:276. In certain embodiments, the VH comprises CDR1, CDR2, and CDR3 comprising the amino acid sequences of SEQ ID NOs: 214, 215, and 223, respectively. In certain embodiments, the VL comprises CDR1, CDR2, and CDR3 comprising the amino acid sequences of SEQ ID NOs:217, 77, and 218, respectively. In certain embodiments, the second antigen-binding site comprises (a) a VH that comprises CDR1, CDR2, and CDR3 comprising the amino acid sequences of SEQ ID NOs: 214, 215, and 223, respectively; and (b) a VL that comprises CDR1, CDR2, and CDR3 comprising the amino acid sequences of SEQ ID NOs:217, 77, and 218, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:278, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:276. In certain embodiments, the VH comprises CDR1, CDR2, and CDR3 comprising the amino acid sequences of SEQ ID NOs: 214, 215, and 224, respectively. In certain embodiments, the VL comprises CDR1, CDR2, and CDR3 comprising the amino acid sequences of SEQ ID NOs:217, 77, and 218, respectively. In certain embodiments, the second antigen-binding site comprises (a) a VH that comprises CDR1, CDR2, and CDR3 comprising the amino acid sequences of SEQ ID NOs: 214, 215, and 224, respectively; and (b) a VL that comprises CDR1, CDR2, and CDR3 comprising the amino acid sequences of SEQ ID NOs: 217, 77, and 218, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:279, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:276. In certain embodiments, the VH comprises CDR1, CDR2, and CDR3 comprising the amino acid sequences of SEQ ID NOs: 214, 215, and 225, respectively. In certain embodiments, the VL comprises CDR1, CDR2, and CDR3 comprising the amino acid sequences of SEQ ID NOs: 217, 77, and 218, respectively. In certain embodiments, the second antigen-binding site comprises (a) a VH that comprises CDR1, CDR2, and CDR3 comprising the amino acid sequences of SEQ ID NOs: 214, 215, and 225, respectively; and (b) a VL that comprises CDR1, CDR2, and CDR3 comprising the amino acid sequences of SEQ ID NOs: 217, 77, and 218, respectively.

In certain embodiments, the VH of the second antigen-binding site that binds BAFF-R comprises CDR1, CDR2, and CDR3 comprising the amino acid sequences of SEQ ID NOs: 231, 215, and 232, respectively. In certain embodiments, the VL of the second antigen-binding site that binds BAFF-R comprises CDR1, CDR2, and CDR3 comprising the amino acid sequences of SEQ ID NOs: 217, 77, and 218, respectively. In certain embodiments, the second antigen-binding site comprises (a) a VH that comprises CDR1, CDR2, and CDR3 comprising the amino acid sequences of SEQ ID NOs: 231, 215, and 232, respectively; and (b) a VL that comprises CDR1, CDR2, and CDR3 comprising the amino acid sequences of SEQ ID NOs: 217, 77, and 218, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:280, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:276. In certain embodiments, the VH comprises CDR1, CDR2, and CDR3 comprising the amino acid sequences of SEQ ID NOs: 227, 215, and 224, respectively. In certain embodiments, the VL comprises CDR1, CDR2, and CDR3 comprising the amino acid sequences of SEQ ID NOs: 217, 77, and 218, respectively. In certain embodiments, the second antigen-binding site comprises (a) a VH that comprises CDR1, CDR2, and CDR3 comprising the amino acid sequences of SEQ ID NOs: 227, 215, and 224, respectively; and (b) a VL that comprises CDR1, CDR2, and CDR3 comprising the amino acid sequences of SEQ ID NOs: 217, 77, and 218, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:281, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:276. In certain embodiments, the VH comprises CDR1, CDR2, and CDR3 comprising the amino acid sequences of SEQ ID NOs: 228, 215, and 229, respectively. In certain embodiments, the VL comprises CDR1, CDR2, and CDR3 comprising the amino acid sequences of SEQ ID NOs: 217, 77, and 218, respectively. In certain embodiments, the second antigen-binding site comprises (a) a VH that comprises CDR1, CDR2, and CDR3 comprising the amino acid sequences of SEQ ID NOs: 228, 215, and 229, respectively; and (b) a VL that comprises CDR1, CDR2, and CDR3 comprising the amino acid sequences of SEQ ID NOs: 217, 77, and 218, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:282, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:276. In certain embodiments, the VH comprises CDR1, CDR2, and CDR3 comprising the amino acid sequences of SEQ ID NOs: 230, 215, and 224, respectively. In certain embodiments, the VL comprises CDR1, CDR2, and CDR3 comprising the amino acid sequences of SEQ ID NOs: 217, 77, and 218, respectively. In certain embodiments, the second antigen-binding site comprises (a) a VH that comprises CDR1, CDR2, and CDR3 comprising the amino acid sequences of SEQ ID NOs: 230, 215, and 224, respectively; and (b) a VL that comprises CDR1, CDR2, and CDR3 comprising the amino acid sequences of SEQ ID NOs: 217, 77, and 218, respectively.

In certain embodiments, the VH of the second antigen-binding site that binds BAFF-R comprises CDR1, CDR2, and CDR3 comprising the amino acid sequences of SEQ ID NOs: 230, 233, and 236, respectively. In certain embodiments, the VL of the second antigen-binding site that binds BAFF-R comprises CDR1, CDR2, and CDR3 comprising the amino acid sequences of SEQ ID NOs: 217, 77, and 218, respectively. In certain embodiments, the second antigen-binding site comprises (a) a VH that comprises CDR1, CDR2, and CDR3 comprising the amino acid sequences of SEQ ID NOs: 230, 233, and 236, respectively; and (b) a VL that comprises CDR1, CDR2, and CDR3 comprising the amino acid sequences of SEQ ID NOs: 217, 77, and 218, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:283, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:276. In certain embodiments, the VH comprises CDR1, CDR2, and CDR3 comprising the amino acid sequences of SEQ ID NOs: 230, 233, and 242, respectively. In certain embodiments, the VL comprises CDR1, CDR2, and CDR3 comprising the amino acid sequences of SEQ ID NOs: 217, 77, and 218, respectively. In certain embodiments, the second antigen-binding site comprises (a) a VH that comprises CDR1, CDR2, and CDR3 comprising the amino acid sequences of SEQ ID NOs: 230, 233, and 242, respectively; and (b) a VL that comprises CDR1, CDR2, and CDR3 comprising the amino acid sequences of SEQ ID NOs: 217, 77, and 218, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:284, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:276. In certain embodiments, the VH comprises CDR1, CDR2, and CDR3 comprising the amino acid sequences of SEQ ID NOs: 230, 233, and 234, respectively. In certain embodiments, the VL comprises CDR1, CDR2, and CDR3 comprising the amino acid sequences of SEQ ID NOs: 217, 77, and 218, respectively. In certain embodiments, the second antigen-binding site comprises (a) a VH that comprises CDR1, CDR2, and CDR3 comprising the amino acid sequences of SEQ ID NOs: 230, 233, and 234, respectively; and (b) a VL that comprises CDR1, CDR2, and CDR3 comprising the amino acid sequences of SEQ ID NOs: 217, 77, and 218, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:285, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:276. In certain embodiments, the VH comprises CDR1, CDR2, and CDR3 comprising the amino acid sequences of SEQ ID NOs: 230, 233, and 235, respectively. In certain embodiments, the VL comprises CDR1, CDR2, and CDR3 comprising the amino acid sequences of SEQ ID NOs: 217, 77, and 218, respectively. In certain embodiments, the second antigen-binding site comprises (a) a VH that comprises CDR1, CDR2, and CDR3 comprising the amino acid sequences of SEQ ID NOs: 230, 233, and 235, respectively; and (b) a VL that comprises CDR1, CDR2, and CDR3 comprising the amino acid sequences of SEQ ID NOs: 217, 77, and 218, respectively.

In certain embodiments, the VH of the second antigen-binding site that binds BAFF-R comprises CDR1, CDR2, and CDR3 comprising the amino acid sequences of SEQ ID NOs: 245, 246, and 247, respectively. In certain embodiments, the VL of the second antigen-binding site that binds BAFF-R comprises CDR1, CDR2, and CDR3 comprising the amino acid sequences of SEQ ID NOs: 217, 77, and 259, respectively. In certain embodiments, the second antigen-binding site comprises (a) a VH that comprises CDR1, CDR2, and CDR3 comprising the amino acid sequences of SEQ ID NOs: 245, 246, and 247, respectively; and (b) a VL that comprises CDR1, CDR2, and CDR3 comprising the amino acid sequences of SEQ ID NOs: 217, 77, and 259, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:286, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:253. In certain embodiments, the VH comprises CDR1, CDR2, and CDR3 comprising the amino acid sequences of SEQ ID NOs: 214, 233, and 237, respectively. In certain embodiments, the VL comprises CDR1, CDR2, and CDR3 comprising the amino acid sequences of SEQ ID NOs: 217, 77, and 249, respectively. In certain embodiments, the second antigen-binding site comprises (a) a VH that comprises CDR1, CDR2, and CDR3 comprising the amino acid sequences of SEQ ID NOs: 214, 233, and 237, respectively; and (b) a VL that comprises CDR1, CDR2, and CDR3 comprising the amino acid sequences of SEQ ID NOs: 217, 77, and 249, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:287, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:253. In certain embodiments, the VH comprises CDR1, CDR2, and CDR3 comprising the amino acid sequences of SEQ ID NOs: 238, 239, and 240, respectively. In certain embodiments, the VL comprises CDR1, CDR2, and CDR3 comprising the amino acid sequences of SEQ ID NOs: 217, 77, and 249, respectively. In certain embodiments, the second antigen-binding site comprises (a) a VH that comprises CDR1, CDR2, and CDR3 comprising the amino acid sequences of SEQ ID NOs: 238, 239, and 240, respectively; and (b) a VL that comprises CDR1, CDR2, and CDR3 comprising the amino acid sequences of SEQ ID NOs: 217, 77, and 249, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:288, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:253. In certain embodiments, the VH comprises CDR1, CDR2, and CDR3 comprising the amino acid sequences of SEQ ID NOs: 241, 233, and 242, respectively. In certain embodiments, the VL comprises CDR1, CDR2, and CDR3 comprising the amino acid sequences of SEQ ID NOs: 217, 77, and 249, respectively. In certain embodiments, the second antigen-binding site comprises (a) a VH that comprises CDR1, CDR2, and CDR3 comprising the amino acid sequences of SEQ ID NOs: 241, 233, and 242, respectively; and (b) a VL that comprises CDR1, CDR2, and CDR3 comprising the amino acid sequences of SEQ ID NOs: 217, 77, and 249, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:289, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:289. In certain embodiments, the VH comprises CDR1, CDR2, and CDR3 comprising the amino acid sequences of SEQ ID NOs: 243, 215, and 244, respectively. In certain embodiments, the VL comprises CDR1, CDR2, and CDR3 comprising the amino acid sequences of SEQ ID NOs: 217, 77, and 249, respectively. In certain embodiments, the second antigen-binding site comprises (a) a VH that comprises CDR1, CDR2, and CDR3 comprising the amino acid sequences of SEQ ID NOs: 243, 215, and 244, respectively; and (b) a VL that comprises CDR1, CDR2, and CDR3 comprising the amino acid sequences of SEQ ID NOs: 217, 77, and 249, respectively.

In certain embodiments, the VH of the second antigen-binding site that binds BAFF-R comprises CDR1, CDR2, and CDR3 comprising the amino acid sequences of SEQ ID NOs: 256, 257 and 258, respectively. In certain embodiments, the VL of the second antigen-binding site that binds BAFF-R comprises CDR1, CDR2, and CDR3 comprising the amino acid sequences of SEQ ID NOs: 217, 77, and 259, respectively. In certain embodiments, the second antigen-binding site comprises (a) a VH that comprises CDR1, CDR2, and CDR3 comprising the amino acid sequences of SEQ ID NOs: 256, 257 and 258, respectively; and (b) a VL that comprises CDR1, CDR2, and CDR3 comprising the amino acid sequences of SEQ ID NOs: 217, 77, and 259, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 250 or 252, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:251 or 253. In certain embodiments, the VH comprises CDR1, CDR2, and CDR3 comprising the amino acid sequences of SEQ ID NOs: 214, 233, and 248, respectively. In certain embodiments, the VL comprises CDR1, CDR2, and CDR3 comprising the amino acid sequences of SEQ ID NOs: 217, 77, and 249, respectively. In certain embodiments, the second antigen-binding site comprises (a) a VH that comprises CDR1, CDR2, and CDR3 comprising the amino acid sequences of SEQ ID NOs: 214, 233, and 248, respectively; and (b) a VL that comprises CDR1, CDR2, and CDR3 comprising the amino acid sequences of SEQ ID NOs: 217, 77, and 249, respectively. In certain embodiments, the second antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:254 or 255.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:263, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:264. In certain embodiments, the VH comprises CDR1, CDR2, and CDR3 comprising the amino acid sequences of SEQ ID NOs: 291, 292, and 293, respectively. In certain embodiments, the VL comprises CDR1, CDR2, and CDR3 comprising the amino acid sequences of SEQ ID NOs: 294, 295, and 296, respectively. In certain embodiments, the second antigen-binding site comprises (a) a VH that comprises CDR1, CDR2, and CDR3 comprising the amino acid sequences of SEQ ID NOs: 291, 292, and 293, respectively; and (b) a VL that comprises CDR1, CDR2, and CDR3 comprising the amino acid sequences of SEQ ID NOs: 294, 295, and 296, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:265, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:266. In certain embodiments, the VH comprises CDR1, CDR2, and CDR3 comprising the amino acid sequences of SEQ ID NOs: 291, 297, and 298, respectively. In certain embodiments, the VL comprises CDR1, CDR2, and CDR3 comprising the amino acid sequences of SEQ ID NOs: 294, 295, and 296, respectively. In certain embodiments, the second antigen-binding site comprises (a) a VH that comprises CDR1, CDR2, and CDR3 comprising the amino acid sequences of SEQ ID NOs: 291, 297, and 298, respectively; and (b) a VL that comprises CDR1, CDR2, and CDR3 comprising the amino acid sequences of SEQ ID NOs: 294, 295, and 296, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:267, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:268. In certain embodiments, the VH comprises CDR1, CDR2, and CDR3 comprising the amino acid sequences of SEQ ID NOs: 299, 300, and 301, respectively. In certain embodiments, the VL comprises CDR1, CDR2, and CDR3 comprising the amino acid sequences of SEQ ID NOs: 302, 303, and 304, respectively. In certain embodiments, the second antigen-binding site comprises (a) a VH that comprises CDR1, CDR2, and CDR3 comprising the amino acid sequences of SEQ ID NOs: 299, 300, and 301, respectively; and (b) a VL that comprises CDR1, CDR2, and CDR3 comprising the amino acid sequences of SEQ ID NOs: 302, 303, and 304, respectively.

In certain embodiments, the second antigen-binding site that binds BAFF-R comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:269, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:262. In certain embodiments, the VH comprises CDR1, CDR2, and CDR3 comprising the amino acid sequences of SEQ ID NOs: 305, 306, and 307, respectively. In certain embodiments, the VL comprises CDR1, CDR2, and CDR3 comprising the amino acid sequences of SEQ ID NOs: 308, 303, and 309, respectively. In certain embodiments, the second antigen-binding site comprises (a) a VH that comprises CDR1, CDR2, and CDR3 comprising the amino acid sequences of SEQ ID NOs: 305, 306, and 307, respectively; and (b) a VL that comprises CDR1, CDR2, and CDR3 comprising the amino acid sequences of SEQ ID NOs: 308, 303, and 309, respectively.

In certain embodiments, the second antigen-binding site that binds BAFF-R is an scFv. For example, in certain embodiments, the second antigen-binding site comprises the amino acid sequence of SEQ ID NO: 207, 138, 139, 140, 141, 142, 143, 144, 149, 190, 191, 192, 254 or 255.

Alternatively, novel antigen-binding sites that can bind to BAFF-R can be identified by screening for binding to the amino acid sequence defined by binding to the amino acid sequence defined by SEQ ID NO:189, a variant thereof, a mature extracellular fragment thereof or a fragment containing a domain of BAFF-R.

SEQ ID NO: 189
MRRGPRSLRGRDAPAPTPCVPAECFDLLVRHCVACGLLRTPRPKPA
GASSPAPRTALQPQESVGAGAGEAALPLPGLLFGAPALLGLALVL
ALVLVGLVSWRRRQRRLRGASSAEAPDGDKDAPEPLDKVIILSPG
ISDATAPAWPPPGEDPGTTPPGHSVPVPATELGSTELVTTKTAGP
EQQ

It is contemplated that in an scFv, a VH and a VL can be connected by a linker, e.g., (GlyGlyGlyGlySer)₄ i.e. (G₄S)₄ linker (SEQ ID NO:119). A skilled person in the art would appreciate that any of the other disclosed linkers (see, e.g., Table 10) may be used in an scFv having a VH and VL sequence disclosed herein (e.g., in Table 2).

In each of the foregoing embodiments, it is contemplated herein that the scFv, VH and/or VL sequences that bind BAFF-R may contain amino acid alterations (e.g., at least 1, 2, 3, 4, 5, or 10 amino acid substitutions, deletions, or additions) in the framework regions of the VH and/or VL without affecting their ability to BAFF-R. For example, it is contemplated herein that scFv, VH and/or VL sequences that bind BAFF-R may contain cysteine heterodimerization mutations, facilitating formation of a disulfide bridge between the VH and VL of the scFv.

In certain embodiments, the second antigen-binding site competes for binding to BAFF-R with a corresponding antigen-binding site described above.

In certain embodiments, the second antigen-binding site blocks interaction of BAFF-R with BAFF ligand.

### Fc Domain

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction. Accordingly, in certain embodiment, the antibody Fc domain or the portion thereof comprises a hinge and a CH2 domain.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, U.S. Ser. No. 13/642,253, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that these conventions consist of nonsequential numbering in specific regions of an immunoglobulin sequence, enabling a normalized reference to conserved positions in immunoglobulin families. Accordingly, the positions of any given immunoglobulin as defined by the EU index or by the Kabat numbering scheme will not necessarily correspond to its sequential sequence.

With knowledge of the residue number according to Kabat or EU index numbering, one of ordinary skill can apply the teachings of the art to identify amino acid sequence modifications within the present invention, according to any commonly used numbering convention. It is understood that the SEQ ID NOs provide sequential numbering of amino acids within a given polypeptide and, thus, may not conform to the corresponding amino acid numbers as provided by Kabat or EU index.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain described in the application can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as a human IgG1 constant region, an IgG2 constant region, IgG3 constant region, or IgG4 constant region. In one embodiment, the antibody Fc domain or a portion thereof sufficient to bind CD16 comprises an amino acid sequence at least 90% (e.g., at least 91%, at least 92%, at least 93%, at least 94%, at least 95%, at least 96%, at least 97%, at least 98%, at least 99%, or 100%) identical to wild-type human IgG1 Fc sequence DKTHTCPPCPAPELLGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPG (SEQ ID NO:118). In some other embodiments, the amino acid sequence of the constant region is at least 90% identical to an antibody constant region from another mammal, such as rabbit, dog, cat, mouse, or horse.

In some embodiments, the antibody constant domain linked to the scFv or the Fab fragment is able to bind to CD16. In some embodiments, the protein incorporates a portion of an antibody Fc domain (for example, a portion of an antibody Fc domain sufficient to bind CD16), wherein the antibody Fc domain comprises a hinge and a CH2 domain (for example, a hinge and a CH2 domain of a human IgG1 antibody), and/or amino acid sequences at least 90% identical to amino acid sequence 234-332 of a human IgG antibody.

One or more mutations can be incorporated into the constant region as compared to human IgG1 constant region, for example at Q347, Y349, L351, S354, E356, E357, K360, Q362, S364, T366, L368, K370, N390, K392, T394, D399, 5400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, F405L, Y407A, Y407I, Y407V, K409F, K409W, K409D, K409R, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cx of a human IgG1 constant region may be at amino acid E123, F116, 5176, V163, S174, and/or T164.

Alternatively, amino acid substitutions could be selected from the following sets of substitutions shown in Table 3.

TABLE 3

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 4.

TABLE 4

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following sets of substitutions shown in Table 5.

TABLE 5

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 6.

TABLE 6

| First Polypeptide | Second Polypeptide |
|---|---|
| L351Y, D399R, D399K, S400K, | T366V, T366I, T366L, T366M, |
| S400R, Y407A, Y407I, Y407V | N390D, N390E, K392L, |
| | K392M, K392V, K392F |
| | K392D, K392E, K409F, |
| | K409W, T411D and T411E |

Alternatively, at least one amino acid substitution could be selected from the following sets of substitutions in Table 7, where the position(s) indicated in the First Polypeptide column is replaced by any known negatively-charged amino acid, and the position(s) indicated in the Second Polypeptide Column is replaced by any known positively-charged amino acid.

TABLE 7

| First Polypeptide | Second Polypeptide |
|---|---|
| K392, K370, K409, or K439 | D399, E356, or E357 |

Alternatively, at least one amino acid substitution could be selected from the following set in Table 8, where the position(s) indicated in the First Polypeptide column is replaced by any known positively-charged amino acid, and the position(s) indicated in the Second Polypeptide Column is replaced by any known negatively-charged amino acid.

TABLE 8

| First Polypeptide | Second Polypeptide |
|---|---|
| D399, E356, or E357 | K409, K439, K370, or K392 |

Alternatively, amino acid substitutions could be selected from the following sets in Table 9.

TABLE 9

| First Polypeptide | Second Polypeptide |
|---|---|
| T350V, L351Y, F405A, | T350V, T366L, K392L, |
| and Y407V | and T394W |

Alternatively, or in addition, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 (e.g., human IgG1) constant region at position T366, and wherein the amino acid sequence of the other polypeptide chain of the antibody constant region differs from the amino acid sequence of an IgG1 (e.g., human IgG1) constant region at one or more positions selected from the group consisting of T366, L368 and Y407.

In some embodiments, the amino acid sequence of one polypeptide chain of the antibody constant region differs from the amino acid sequence of an IgG1 (e.g., human IgG1) constant region at one or more positions selected from the group consisting of T366, L368 and Y407, and wherein the amino acid sequence of the other polypeptide chain of the antibody constant region differs from the amino acid sequence of an IgG1 (e.g., human IgG1) constant region at position T366.

In some embodiments, the amino acid sequence of one polypeptide chain of the antibody constant region differs from the amino acid sequence of an IgG1 (e.g., human IgG1) constant region at one or more positions selected from the group consisting of E357, K360, Q362, S364, L368, K370, T394, D401, F405, and T411 and wherein the amino acid sequence of the other polypeptide chain of the antibody constant region differs from the amino acid sequence of an IgG1 (e.g., human IgG1) constant region at one or more positions selected from the group consisting of Y349, E357, S364, L368, K370, T394, D401, F405 and T411.

In some embodiments, the amino acid sequence of one polypeptide chain of the antibody constant region differs from the amino acid sequence of an IgG1 (e.g., human IgG1) constant region at one or more positions selected from the group consisting of Y349, E357, S364, L368, K370, T394, D401, F405 and T411 and wherein the amino acid sequence of the other polypeptide chain of the antibody constant region differs from the amino acid sequence of an IgG1 (e.g., human IgG1) constant region at one or more positions selected from the group consisting of E357, K360, Q362, S364, L368, K370, T394, D401, F405, and T411.

In some embodiments, the amino acid sequence of one polypeptide chain of the antibody constant region differs from the amino acid sequence of an IgG1 (e.g., human IgG1) constant region at one or more positions selected from the group consisting of L351, D399, S400 and Y407 and wherein the amino acid sequence of the other polypeptide chain of the antibody constant region differs from the amino acid sequence of an IgG1 (e.g., human IgG1) constant region at one or more positions selected from the group consisting of T366, N390, K392, K409 and T411.

In some embodiments, the amino acid sequence of one polypeptide chain of the antibody constant region differs from the amino acid sequence of an IgG1 (e.g., human IgG1) constant region at one or more positions selected from the group consisting of T366, N390, K392, K409 and T411 and wherein the amino acid sequence of the other polypeptide chain of the antibody constant region differs from the amino acid sequence of an IgG1 (e.g., human IgG1) constant region at one or more positions selected from the group consisting of L351, D399, S400 and Y407.

In some embodiments, the amino acid sequence of one polypeptide chain of the antibody constant region differs from the amino acid sequence of an IgG1 (e.g., human IgG1) constant region at one or more positions selected from the group consisting of Q347, Y349, K360, and K409, and wherein the amino acid sequence of the other polypeptide chain of the antibody constant region differs from the amino acid sequence of an IgG1 (e.g., human IgG1) constant region at one or more positions selected from the group consisting of Q347, E357, D399 and F405.

In some embodiments, the amino acid sequence of one polypeptide chain of the antibody constant region differs from the amino acid sequence of an IgG1 (e.g., human IgG1) constant region at one or more positions selected from the group consisting of Q347, E357, D399 and F405, and wherein the amino acid sequence of the other polypeptide chain of the antibody constant region differs from the amino acid sequence of an IgG1 (e.g., human IgG1) constant region at one or more positions selected from the group consisting of Y349, K360, Q347 and K409.

In some embodiments, the amino acid sequence of one polypeptide chain of the antibody constant region differs from the amino acid sequence of an IgG1 (e.g., human IgG1) constant region at one or more positions selected from the group consisting of K370, K392, K409 and K439, and wherein the amino acid sequence of the other polypeptide chain of the antibody constant region differs from the amino acid sequence of an IgG1 (e.g., human IgG1) constant region at one or more positions selected from the group consisting of D356, E357 and D399.

In some embodiments, the amino acid sequence of one polypeptide chain of the antibody constant region differs from the amino acid sequence of an IgG1 (e.g., human IgG1) constant region at one or more positions selected from the group consisting of D356, E357 and D399, and wherein the amino acid sequence of the other polypeptide chain of the antibody constant region differs from the amino acid sequence of an IgG1 (e.g., human IgG1) constant region at one or more positions selected from the group consisting of K370, K392, K409 and K439.

In some embodiments, the amino acid sequence of one polypeptide chain of the antibody constant region differs from the amino acid sequence of an IgG1 (e.g., human IgG1) constant region at one or more positions selected from the group consisting of L351, E356, T366 and D399, and wherein the amino acid sequence of the other polypeptide chain of the antibody constant region differs from the amino acid sequence of an IgG1 (e.g., human IgG1) constant region at one or more positions selected from the group consisting of Y349, L351, L368, K392 and K409.

In some embodiments, the amino acid sequence of one polypeptide chain of the antibody constant region differs from the amino acid sequence of an IgG1 (e.g., human IgG1) constant region at one or more positions selected from the group consisting of Y349, L351, L368, K392 and K409, and wherein the amino acid sequence of the other polypeptide chain of the antibody constant region differs from the amino acid sequence of an IgG1 (e.g., human IgG1) constant region at one or more positions selected from the group consisting of L351, E356, T366 and D399.

In some embodiments, the amino acid sequence of one polypeptide chain of the antibody constant region differs from the amino acid sequence of an IgG1 (e.g., human IgG1) constant region by an S354C substitution and wherein the amino acid sequence of the other polypeptide chain of the antibody constant region differs from the amino acid sequence of an IgG1 (e.g., human IgG1) constant region by a Y349C substitution.

In some embodiments, the amino acid sequence of one polypeptide chain of the antibody constant region differs from the amino acid sequence of an IgG1 (e.g., human IgG1) constant region by a Y349C substitution and wherein the amino acid sequence of the other polypeptide chain of the antibody constant region differs from the amino acid sequence of an IgG1 (e.g., human IgG1) constant region by an S354C substitution.

In some embodiments, the amino acid sequence of one polypeptide chain of the antibody constant region differs from the amino acid sequence of an IgG1 (e.g., human IgG1) constant region by K360E and K409W substitutions and wherein the amino acid sequence of the other polypeptide chain of the antibody constant region differs from the amino acid sequence of an IgG1 (e.g., human IgG1) constant region by Q347R, D399V and F405T substitutions.

In some embodiments, the amino acid sequence of one polypeptide chain of the antibody constant region differs from the amino acid sequence of an IgG1 (e.g., human IgG1) constant region by Q347R, D399V and F405T substitutions and wherein the amino acid sequence of the other polypeptide chain of the antibody constant region differs from the amino acid sequence of an IgG1 (e.g., human IgG1) constant region by K360E and K409W substitutions.

In some embodiments, the amino acid sequence of one polypeptide chain of the antibody constant region differs from the amino acid sequence of an IgG1 (e.g., human IgG1) constant region by a T366W substitutions and wherein the amino acid sequence of the other polypeptide chain of the antibody constant region differs from the amino acid sequence of an IgG1 (e.g., human IgG1) constant region by T366S, T368A, and Y407V substitutions.

In some embodiments, the amino acid sequence of one polypeptide chain of the antibody constant region differs from the amino acid sequence of an IgG1 (e.g., human IgG1) constant region by T366S, T368A, and Y407V substitutions and wherein the amino acid sequence of the other polypeptide chain of the antibody constant region differs from the amino acid sequence of an IgG1 (e.g., human IgG1) constant region by a T366W substitution.

In some embodiments, the amino acid sequence of one polypeptide chain of the antibody constant region differs from the amino acid sequence of an IgG1 (e.g., human IgG1) constant region by T350V, L351Y, F405A, and Y407V substitutions and wherein the amino acid sequence of the other polypeptide chain of the antibody constant region differs from the amino acid sequence of an IgG1 (e.g., human IgG1) constant region by T350V, T366L, K392L, and T394W substitutions.

In some embodiments, the amino acid sequence of one polypeptide chain of the antibody constant region differs from the amino acid sequence of an IgG1 (e.g., human IgG1) constant region by T350V, T366L, K392L, and T394W substitutions and wherein the amino acid sequence of the other polypeptide chain of the antibody constant region differs from the amino acid sequence of an IgG1 (e.g., human IgG1) constant region by T350V, L351Y, F405A, and Y407V substitutions.

In some embodiments, the amino acid sequence of one polypeptide chain of the antibody constant region differs from the amino acid sequence of an IgG1 (e.g., human IgG1) constant region by an F405L substitution and wherein the amino acid sequence of the other polypeptide chain of the antibody constant region differs from the amino acid sequence of an IgG1 (e.g., human IgG1) constant region by a K409R substitution.

### Exemplary Multispecific Binding Proteins

Listed below are examples of TriNKETs comprising an antigen-binding site that binds BAFF-R and an antigen-binding site that binds NKG2D each linked to an antibody constant region, wherein the antibody constant regions include mutations that enable heterodimerization of two Fc chains.

Exemplary BAFF-R-targeting TriNKETs are contemplated in the F3′, F4, and 2-Fab formats. As described above, in the F3′ format, the antigen-binding site that binds BAFF-R is an scFv and the antigen-binding site that binds NKG2D is a Fab. In the F4 format, the antigen binding-sites that bind BAFF-R are Fab fragments and the antigen-binding site that binds NKG2D is an scFv. In each TriNKET, the scFv may comprise substitution of Cys in the VH and VL regions, facilitating formation of a disulfide bridge between the VH and VL of the scFv. In the 2-Fab format, both the antigen-binding site that binds BAFF-R and the antigen-binding site that binds NKG2D are Fabs.

The VH and VL of an scFv can be connected via a linker, e.g., a peptide linker. In certain embodiments, the peptide linker is a flexible linker. Regarding the amino acid composition of the linker, peptides are selected with properties that confer flexibility, do not interfere with the structure and function of the other domains of the proteins described in the present application, and resist cleavage from proteases. For example, glycine and serine residues generally provide protease resistance. In certain embodiments, the VL is linked N-terminal or C-terminal to the VH via a (GlyGlyGlyGlySer)₄ ((G₄S)₄) linker (SEQ ID NO:119).

The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, or 20-25 amino acid residues in length.

In certain embodiments, the linker comprises or consists of a (GS)ₙ (SEQ ID NO:120), (GGS)ₙ (SEQ ID NO:121), (GGGS)ₙ (SEQ ID NO:122), (GGSG)ₙ (SEQ ID NO:123), (GGSGG)ₙ (SEQ ID NO:124), and (GGGGS)ₙ (SEQ ID NO:125) sequence, wherein n is 1, 2, 3, 4, 5, 6, 7, 8, 9, 10, 11, 12, 13, 14, 15, 16, 17, 18, 19, or 20. In certain embodiments, the linker comprises or consists of an amino acid sequence selected from SEQ ID NO:119 and SEQ ID NO:126-134, as listed in Table 10.

TABLE 10

| SEQ ID | Amino Acid Sequence |
|---|---|
| SEQ ID NO: 126 | GSGSGSGSGSGSGSGSGSGS |
| SEQ ID NO: 127 | GGSGGSGGSGGSGGSGGSGGSGGSGGSGGS |
| SEQ ID NO: 128 | GGGSGGGSGGGSGGGSGGGSGGGSGGGSGGGSGGGSGGGS |
| SEQ ID NO: 129 | GGSGGGSGGGSGGGSGGGSGGGSGGGSGGGSGGGSGGGSG |
| SEQ ID NO: 130 | GGSGGGGSGGGGSGGGGSGGGGSGGGGSGGGGSGGGGSGGG |
| | GSGGGGSGG |
| SEQ ID NO: 131 | GGGGSGGGGSGGGGSGGGGSGGGGSGGGGSGGGGSGGGGSG |
| | GGGSGGGGS |
| SEQ ID NO: 119 | GGGGSGGGGSGGGGSGGGGS |
| SEQ ID NO: 132 | GGGGSGGGGSGGGGS |
| SEQ ID NO: 133 | GGGGSGGGGSGGGGSGGGGSGGGGSGGGGSGGGGSGGGGSG |
| | GGGSGGGGSGGGGSGGGGSGGGGSGGGGSGGGGSGGGGSGG |
| | GGSGGGGSGGGGSGGGGS |
| SEQ ID NO: 134 | GGSGGGGSGGGGSGGGGSGGGGSGGGGSGGGGSGGGGSGGG |
| | GSGGGGSGGGGSGGGGSGGGGSGGGGSGGGGSGGGGSGGGGS |
| | GGGGSGGGGSGGGGSGG |
| SEQ ID NO: 274 | SGSGGGGS |

In the F3′-TriNKETs, the BAFF-R binding scFv is linked to the N-terminus of an Fc via an Ala-Ser or Gly-Ser linker. The Ala-Ser or Gly-Ser linker is included at the elbow hinge region sequence to balance between flexibility and optimal geometry. In certain embodiments, an additional amino acid sequence Thr-Lys-Gly can be added N-terminal or C-terminal to the Ala-Ser or Gly-Ser sequence at the hinge. In the F4 TriNKETs, the NKG2D-binding scFv is linked to the C-terminus of an Fc via a short linker comprising the amino acid sequence SGSGGGGS (SEQ ID NO:274).

As used herein to describe these exemplary TriNKETs, an Fc includes an antibody hinge, CH2, and CH3. In each exemplary TriNKET, the Fc domain linked to an scFv comprises the mutations of Q347R, D399V, and F405T, and the Fc domain linked to a Fab comprises matching mutations K360E and K409W for forming a heterodimer. The Fc domain linked to the scFv further includes an S354C substitution in the CH3 domain, which forms a disulfide bond with a Y349C substitution on the Fc linked to the Fab. These substitutions are bold in the sequences described in this subsection.

For example, a TriNKET described in the present disclosure is ianalumab-F3′. Ianalumab-F3′ includes (a) a BAFF-R-binding scFv sequence comprising the VH and VL sequences of ianalumab described of Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. Ianalumab-F3′ includes three polypeptides: scFv-ianalumab-VL-VH-Fc (SEQ ID NO:193), A49MI-VH-CH1-Fc (SEQ ID NO:194 and A49MI-VL-CL (SEQ ID NO:195).

scFv-ianalumab-VL-VH-Fc (“Chain S”)
(SEQ ID NO: 193)
DIVLTQSPATLSLSPGERATLSCRASQFILPEYLSWYQQKPGQAP
RLLIYGSSSRATGVPARFSGSGSGTDFTLTISSLEPEDFAVYYCQ
QFYSSPLTFGCGTKVEIKGGGGSGGGGSGGGGSGGGGSQVQLQQS
GPGLVKPSQTLSLTCAISGDSVSSNSAAWGWIRQSPGRCLEWLGR
IYYRSKWYNSYAVSVKSRITINPDTSKNQFSLQLNSVTPEDTAVY
YCARYQWVPKIGVFDSWGQGTLVTVSSASDKTHTCPPCPAPELLG
GPSVFLFPPKPKDTLMISRTPEVTCVVVDVSHEDPEVKFNWYVDG
VEVHNAKTKPREEQYNSTYRVVSVLTVLHQDWLNGKEYKCKVSNK
ALPAPIEKTISKAKGQPREPRVYTLPPCRDELTKNQVSLTCLVKG
FYPSDIAVEWESNGQPENNYKTTPPVLVSDGSFTLYSKLTVDKSR
WQQGNVFSCSVMHEALHNHYTQKSLSLSPG
A49MI-VH-CHI-Fc (“Chain H”)
(SEQ ID NO: 194)
EVQLVESGGGLVKPGGSLRLSCAASGFTESSYSMNWVRQAPGKGL
EWVSSISSSSSYIYYADSVKGRFTISRDNAKNSLYLQMNSLRAED
TAVYYCARGAPIGAAAGWFDPWGQGTLVTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKKVEPKSC
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
DELTENQVSLTCLVKGFYPSDIAVEWESNGQPENNYKTTPPVLDS
DGSFFLYSWLTVDKSRWQQGNVFSCSVMHEALHNHYTQKSLSLSP
G
A49MI-VL-CL (“Chain L”)
(SEQ ID NO: 195)
DIQMTQSPSSVSASVGDRVTITCRASQGISSWLAWYQQKPGKAPK
LLIYAASSLQSGVPSRFSGSGSGTDFTLTISSLQPEDFATYYCQQ
GVSFPRTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC

scFv-ianalumab-VL-VH-Fc (SEQ ID NO:193) represents the full sequence of a BAFF-R binding scFv linked to an Fc domain via a hinge comprising Ala-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:207, which includes a heavy chain variable domain of ianalumab connected to the C-terminus of a light chain variable domain of ianalumab via a (G₄S)₄ linker. The scFv comprises substitution of Cys in the VH and VL regions at G44 and Q100, facilitating formation of a disulfide bridge between the VH and VL of the scFv.

A49MI-VH-CH1-Fc (SEQ ID NO:194) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-ianalumab-VL-VH-Fc. In A49MI-VH-CH1-Fc, the Fc domain also includes K360E and K409W substitutions for heterodimerization with the Fc in scFv-ianalumab-VL-VH-Fc.

A49MI-VL-CL (SEQ ID NO:195) represents the light chain portion of the Fab fragment comprising a light chain variable domain of NKG2D-binding A49MI (SEQ ID NO:85) and a light chain constant domain.

Another TriNKET described in the present disclosure is ianalumab-2-Fab. Ianalumab-2-Fab includes (a) a BAFF-R-binding Fab fragment comprising a VH sequence and a VL sequences of ianalumab described in Table 2, including a heavy chain portion comprising a heavy chain variable domain and a CH1 domain, and a light chain portion comprising a light chain variable domain and a light chain constant domain, wherein the CH1 domain is connected to an Fc domain (which does not include antibody-dependent cellular cytotoxicity-enhancing mutations present in the commercial ianalumab antibody); (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. Ianalumab-2-Fab includes four polypeptides: ianalumab-VH-CH1-Fc-Genmab, ianalumab-VL-CL, A49MI-VH-CH1-Fc, and A49MI-VL-CL-Genmab.

Ianalumab-VH-CH1-Fc-Genmab
(SEQ ID NO: 196)
QVQLQQSGPGLVKPSQTLSLTCAISGDSVSSNSAAWGWIRQSPGRGLEW
LGRIYYRSKWYNSYAVSVKSRITINPDTSKNQFSLQLNSVTPEDTAVYY
CARYQWVPKIGVFDSWGQGTLVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFLLYSKLTVDKSRWQQGNVFSCSVMHEALHN
HYTQKSLSLSPG
Ianalumab-VL-CL
(SEQ ID NO: 197)
DIVLTQSPATLSLSPGERATLSCRASQFILPEYLSWYQQKPGQAPRLLI
YGSSSRATGVPARFSGSGSGTDFTLTISSLEPEDFAVYYCQQFYSSPLT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC
A49MI-VH-CH1-Fc-Genmab
(SEQ ID NO: 213)
EVQLVESGGGLVKPGGSLRLSCAASGFTFSSYSMNWVRQAPGKGLEWVS
SISSSSSYIYYADSVKGRFTISRDNAKNSLYLQMNSLRAEDTAVYYCAR
GAPIGAAAGWEDPWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQP
ENNYKTTPPVLDSDGSFFLYSRLTVDKSRWQQGNVFSCSVMHEALHNHY
TQKSLSLSPG
A49MI-VL-CL
(SEQ ID NO: 195)
DIQMTQSPSSVSASVGDRVTITCRASQGISSWLAWYQQKPGKAPKLLIY
AASSLQSGVPSRFSGSGSGTDFTLTISSLQPEDFATYYCQQGVSFPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Ianalumab-VH-CH1-Fc-Genmab (SEQ ID NO:196) represents the heavy chain portion of the Fab fragment, which comprises a heavy chain variable domain (SEQ ID NO:145) of BAFF-R-binding ianalumab and a CH1 domain, connected to an Fc domain. The Fc domain in ianalumab-VH-CH1-Fc-Genmab includes an F405L substitution for heterodimerization with the Fc in A49MI-VH-CH1-Fc-Genmab, which includes a K409R substitution.

Ianalumab-VL-CL (SEQ ID NO:197) represents the light chain portion of the Fab fragment comprising a light chain variable domain of BAFF-R-binding ianalumab (SEQ ID NO:146) and a light chain constant domain.

A49MI-VH-CH1-Fc-Genmab (SEQ ID NO:213) comprises a heavy chain variable domain of NKG2D-binding A49MI (SEQ ID NO:95) and a CH1 domain, connected to an Fc domain. The Fc domain in A49MI-VH-CH1-Fc-Genmab includes a K409R substitution for heterodimerization with the Fc in ianalumab-VH-CH1-Fc-Genmab, which includes an F405L substitution.

A49MI-VL-CL (SEQ ID NO:195), as described above, comprises a light chain variable domain of NKG2D-binding A49MI (SEQ ID NO:85) and a light chain constant domain.

Another exemplary TriNKET described in the present disclosure is hCOH-1-F3′ TriNKET. hCOH-1-F3′ includes (a) a BAFF-R-binding scFv sequence derived from hCOH-1 of Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. hCOH-1-F3′ includes three polypeptides: scFv-hCOH-1-VL-VH-Fc, A49MI-VH-CH1-Fc, and A49MI-VL-CL.

scFv-hCOH-1-VL-VH-Fc (“Chain S”)
(SEQ ID NO: 198)
EIVLTQSPATLSLSPGERATLSCRASESVDNYGISFLNWFQQKPGQAPR
LLIYAASNRATGIPARFSGSGSGTDFTLTISSLEPEDFAVYYCQQSKEV
PWTFGCGTKVEIKGGGGSGGGGSGGGGSGGGGSQVQLQESGPGLVKPSQ
TLSLTCTVSGDSITSGYWNWIRQHPGKCLEYIGYISYSGSTYYNPSLKS
RVTISRDTSKNQFSLKLSSVTAADTAVYYCASPNYPFYAMDYWGQGTLV
TVSSASDKTHTCPPCPAPELLGGPSVFLFPPKPKDTLMISRTPEVTCVV
VDVSHEDPEVKFNWYVDGVEVHNAKTKPREEQYNSTYRVVSVLTVLHQD
WLNGKEYKCKVSNKALPAPIEKTISKAKGQPREPRVYTLPPCRDELTKN
QVSLTCLVKGFYPSDIAVEWESNGQPENNYKTTPPVLVSDGSFTLYSKL
TVDKSRWQQGNVFSCSVMHEALHNHYTQKSLSLSPG

scFv-hCOH-1-VL-VH-Fc (SEQ ID NO:198) represents the full sequence of a BAFF-R binding scFv linked to an Fc domain via a hinge comprising Ala-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:149, which includes a heavy chain variable domain of hCOH-1 connected to the C-terminus of a light chain variable domain of hCOH-1 via a (G₄S)₄ linker. The scFv comprises substitution of Cys in the VH and VL regions at G44 and G100, facilitating formation of a disulfide bridge between the VH and VL of the scFv.

A49MI-VH-CH1-Fc (SEQ ID NO:194), as described above, comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-hCOH-1-VL-VH-Fc. In A49MI-VH-CH1-Fc, the Fc domain also includes K360E and K409W substitutions for heterodimerization with the Fc in scFv-hCOH-1-VL-VH-Fc.

A49MI-VL-CL (SEQ ID NO:195), as described above, comprises a light chain variable domain of NKG2D-binding A49MI (SEQ ID NO:85) and a light chain constant domain.

Another TriNKET described in the present disclosure is hCOH-1-2-Fab. hCOH-1-2-Fab includes (a) a BAFF-R-binding Fab fragment derived from hCOH-1, including a heavy chain portion comprising a heavy chain variable domain and a CH1 domain, and a light chain portion comprising a light chain variable domain and a light chain constant domain, wherein the CH1 domain is connected to an Fc domain; (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. hCOH-1-2-Fab includes four polypeptides: hCOH-1-VH-CH1-Fc-Genmab, hCOH-1-VL-CL, A49MI-VH-CH1-Fc-Genmab, and A49MI-VL-CL.

hCOH-1-VH-CH1-Fc-Genmab
(SEQ ID NO: 208)
QVQLQESGPGLVKPSQTLSLTCTVSGDSITSGYWNWIRQHPGKGLEYIG
YISYSGSTYYNPSLKSRVTISRDTSKNQFSLKLSSVTAADTAVYYCASP
NYPFYAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFLLYSKLTVDKSRWQQGNVFSCSVMHEALHNHYTQKS
LSLSPG
hCOH-1-VL-CL
(SEQ ID NO: 209)
EIVLTQSPATLSLSPGERATLSCRASESVDNYGISFLNWFQQKPGQAPR
LLIYAASNRATGIPARFSGSGSGTDFTLTISSLEPEDFAVYYCQQSKEV
PWTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

hCOH-1-VH-CH1-Fc-Genmab (SEQ ID NO:208) represents the heavy chain portion of the Fab fragment, which comprises a heavy chain variable domain of BAFF-R-binding hCOH-1 (SEQ ID NO:153) and a CH1 domain, connected to an Fc domain. The Fc domain in hCOH-1-VH-CH1-Fc-Genmab includes an F405L substitution for heterodimerization with the Fc in A49MI-VH-CH1-Fc-Genmab, which includes a K409R substitution.

hCOH-1-VL-CL (SEQ ID NO:209) represents the light chain portion of the Fab fragment comprising a light chain variable domain of BAFF-R-binding hCOH-1 (SEQ ID NO:154) and a light chain constant domain.

A49MI-VH-CH1-Fc-Genmab (SEQ ID NO:213) comprises a heavy chain variable domain of NKG2D-binding A49MI (SEQ ID NO:95) and a CH1 domain, connected to an Fc domain. The Fc domain in A49MI-VH-CH1-Fc-Genmab includes a K409R substitution for heterodimerization with the Fc in hCOH-1-VH-CH1-Fc-Genmab, which includes an F405L substitution.

A49MI-VL-CL (SEQ ID NO:195) comprises a light chain variable domain of NKG2D-binding A49MI (SEQ ID NO:85) and a light chain constant domain.

Another exemplary TriNKET described in the present disclosure is hCOH-2-F3′. hCOH-2-F3′ includes (a) a BAFF-R-binding scFv sequence derived from hCOH-2 of Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. hCOH-2-F3′ includes three polypeptides: scFv-hCOH-1-VL-VH-Fc, A49MI-VH-CH1-Fc, and A49MI-VL-CL.

scFv-hCOH-2-VL-VH-Fc (“Chain S”)
(SEQ ID NO: 210)
DIVLTQSPATLSLSPGERATLSCRASESVDNYGISFMNWFQQKPGQAPR
LLIYAASNRATGIPARFSGSGSGTDFTLTISSLEPEDFAVYYCQQSKEV
PWTFGCGTKVEIKGGGGSGGGGSGGGGSGGGGSEVQLQESGPGLVKPSQ
TLSLTCTVSGDSITSGYWNWIRQHPGKCLEYIGYISYSGSTYYNPSLKS
RVTISRDTSKNQYSLKLSSVTAADTAVYYCASPNYPFYAMDYWGQGTLV
TVSSASDKTHTCPPCPAPELLGGPSVFLFPPKPKDTLMISRTPEVTCVV
VDVSHEDPEVKFNWYVDGVEVHNAKTKPREEQYNSTYRVVSVLTVLHQD
WLNGKEYKCKVSNKALPAPIEKTISKAKGQPREPRVYTLPPCRDELTKN
QVSLTCLVKGFYPSDIAVEWESNGQPENNYKTTPPVLVSDGSFTLYSKL
TVDKSRWQQGNVFSCSVMHEALHNHYTQKSLSLSPG

scFv-hCOH-2-VL-VH-Fc (SEQ ID NO:210) represents the full sequence of a BAFF-R binding scFv linked to an Fc domain via a hinge comprising Ala-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:191, which includes a heavy chain variable domain of hCOH-2 connected to the C-terminus of a light chain variable domain of hCOH-2 via a (G₄S)₄ linker. The scFv comprises substitution of Cys in the VH and VL regions at G44 and G100, facilitating formation of a disulfide bridge between the VH and VL of the scFv.

A49MI-VH-CH1-Fc (SEQ ID NO:194) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-hCOH-2-VL-VH-Fc. In A49MI-VH-CH1-Fc, the Fc domain also includes K360E and K409W substitutions for heterodimerization with the Fc in scFv-hCOH-2-VL-VH-Fc.

A49MI-VL-CL (SEQ ID NO:195) represents the light chain portion of the Fab fragment comprising a light chain variable domain of NKG2D-binding A49MI (SEQ ID NO:85) and a light chain constant domain.

Another TriNKET described in the present disclosure is hCOH-2-2-Fab. hCOH-2-2-Fab includes (a) a BAFF-R-binding Fab fragment derived from hCOH-2, including a heavy chain portion comprising a heavy chain variable domain and a CH1 domain, and a light chain portion comprising a light chain variable domain and a light chain constant domain, wherein the CH1 domain is connected to an Fc domain; (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. hCOH-2-2-Fab includes four polypeptides: hCOH-2-VH-CH1-Fc-Genmab, hCOH-2-VL-CL, A49MI-VH-CH1-Fc-Genmab, and A49MI-VL-CL.

hCOH-2-VH-CH1-Fc-Genmab
(SEQ ID NO: 199)
EVQLQESGPGLVKPSQTLSLTCTVSGDSITSGYWNWIRQHPGKGLEYIG
YISYSGSTYYNPSLKSRVTISRDTSKNQYSLKLSSVTAADTAVYYCASP
NYPFYAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFLLYSKLTVDKSRWQQGNVFSCSVMHEALHNHYTQKS
LSLSPG
hCOH-2-VL-CL
(SEQ ID NO: 200)
DIVLTQSPATLSLSPGERATLSCRASESVDNYGISFMNWFQQKPGQAPR
LLIYAASNRATGIPARFSGSGSGTDFTLTISSLEPEDFAVYYCQQSKEV
PWTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

hCOH-2-VH-CH1-Fc-Genmab (SEQ ID NO:199) represents the heavy chain portion of the Fab fragment, which comprises a heavy chain variable domain of BAFF-R-binding hCOH-2 (SEQ ID NO:155) and a CH1 domain, connected to an Fc domain. The Fc domain in hCOH-2-VH-CH1-Fc-Genmab includes an F405L substitution for heterodimerization with the Fc in A49MI-VH-CH1-Fc-Genmab, which includes a K409R substitution.

hCOH-2-VL-CL (SEQ ID NO:200) represents the light chain portion of the Fab fragment comprising a light chain variable domain of BAFF-R-binding hCOH-2 (SEQ ID NO:156) and a light chain constant domain.

A49MI-VH-CH1-Fc-Genmab (SEQ ID NO:213) comprises a heavy chain variable domain of NKG2D-binding A49MI (SEQ ID NO:95) and a CH1 domain, connected to an Fc domain. The Fc domain in A49MI-VH-CH1-Fc-Genmab includes a K409R substitution for heterodimerization with the Fc in hCOH-2-VH-CH1-Fc-Genmab, which includes an F405L substitution.

A49MI-VL-CL (SEQ ID NO:195) comprises a light chain variable domain of NKG2D-binding A49MI (SEQ ID NO:85) and a light chain constant domain.

Another exemplary TriNKET described in the present disclosure is V3-46s-F3′. V3-46s-F3′ includes (a) a BAFF-R-binding scFv sequence derived from V3-46s of Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. V3-46s-F3′ includes three polypeptides: scFv-hCOH-1-VL-VH-Fc, A49MI-VH-CH1-Fc, and A49MI-VL-CL.

scFv-V3-46s-VL-VH-Fc (“Chain S”)
(SEQ ID NO: 201)
DIQMTQSPSSLSASVGDRVTITCRASQDVSTAVAWYQQKPGKAPKLLIY
SASFLYSGVPSRFSGSGSGTDFTLTISSLQPEDFATYYCQQSQISPPTF
GCGTKVEIKGGGGSGGGGSGGGGSGGGGSEVQLVESGGGLVQPGGSLRL
SCAASGFTISSSSIHWVRQAPGKCLEWVAWVLPSVGFTDYADSVKGRFT
ISADTSKNTAYLQMNSLRAEDTAVYYCARRVCYNRLGVCAGGMDYWGQG
TLVTVSSASDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPRVYTLPPCRDEL
TKNQVSLTCLVKGFYPSDIAVEWESNGQPENNYKTTPPVLVSDGSFTLY
SKLTVDKSRWQQGNVFSCSVMHEALHNHYTQKSLSLSPG

scFv-V3-46s-VL-VH-Fc (SEQ ID NO:201) represents the full sequence of a BAFF-R binding scFv linked to an Fc domain via a hinge comprising Ala-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:139, which includes a heavy chain variable domain of V3-46s connected to the C-terminus of a light chain variable domain of V3-46s via a (G₄S)₄ linker. The scFv comprises substitution of Cys in the VH and VL regions at G44 and Q100, facilitating formation of a disulfide bridge between the VH and VL of the scFv.

A49MI-VH-CH1-Fc (SEQ ID NO:194) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-V3-46s-VL-VH-Fc. In A49MI-VH-CH1-Fc, the Fc domain also includes K360E and K409W substitutions for heterodimerization with the Fc in scFv-V3-46s-VL-VH-Fc.

A49MI-VL-CL (SEQ ID NO:195) represents the light chain portion of the Fab fragment comprising a light chain variable domain of NKG2D-binding A49MI (SEQ ID NO:85) and a light chain constant domain.

Another TriNKET described in the present disclosure is V3-46s-2-Fab. V3-46s-2-Fab includes (a) a BAFF-R-binding Fab fragment derived from V3-46s, including a heavy chain portion comprising a heavy chain variable domain and a CH1 domain, and a light chain portion comprising a light chain variable domain and a light chain constant domain, wherein the CH1 domain is connected to an Fc domain; (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. V3-46s-2-Fab includes four polypeptides: V3-46s-VH-CH1-Fc-Genmab, V3-46s-VL-CL, A49MI-VH-CH1-Fc-Genmab, and A49MI-VL-CL.

V3-46s-VH-CH1-Fc-Genmab
(SEQ ID NO: 202)
EVQLVESGGGLVQPGGSLRLSCAASGFTISSSSIHWVRQAPGKGLEWVA
WVLPSVGFTDYADSVKGRFTISADTSKNTAYLQMNSLRAEDTAVYYCAR
RVCYNRLGVCAGGMDYWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFLLYSKLTVDKSRWQQGNVFSCSVMHEALH
NHYTQKSLSLSPG
V3-46s-VL-CL
(SEQ ID NO: 203)
DIQMTQSPSSLSASVGDRVTITCRASQDVSTAVAWYQQKPGKAPKLLIY
SASFLYSGVPSRFSGSGSGTDFTLTISSLQPEDFATYYCQQSQISPPTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

V3-46s-VH-CH1-Fc-Genmab (SEQ ID NO:202) represents the heavy chain portion of the Fab fragment, which comprises a heavy chain variable domain of BAFF-R-binding V3-46s (SEQ ID NO:147) and a CH1 domain, connected to an Fc domain. The Fc domain in V3-46s-VH-CH1-Fc-Genmab includes an F405L substitution for heterodimerization with the Fc in A49MI-VH-CH1-Fc-Genmab, which includes a K409R substitution.

V3-46s-VL-CL (SEQ ID NO:203) represents the light chain portion of the Fab fragment comprising a light chain variable domain of BAFF-R-binding V3-46s (SEQ ID NO:148) and a light chain constant domain.

A49MI-VH-CH1-Fc-Genmab (SEQ ID NO:213) comprises a heavy chain variable domain of NKG2D-binding A49MI (SEQ ID NO:95) and a CH1 domain, connected to an Fc domain. The Fc domain in A49MI-VH-CH1-Fc-Genmab includes a K409R substitution for heterodimerization with the Fc in V3-46s-VH-CH1-Fc-Genmab, which includes an F405L substitution.

A49MI-VL-CL (SEQ ID NO:195) comprises a light chain variable domain of NKG2D-binding A49MI (SEQ ID NO:85) and a light chain constant domain.

Another exemplary TriNKET described in the present disclosure is V3-46s-42-F3′. V3-46s-42-F3′ includes (a) a BAFF-R-binding scFv sequence derived from V3-46s-42 of Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. V3-46s-42-F3′ includes three polypeptides: scFv-V3-46s-42-VL-VH-Fc, A49MI-VH-CH1-Fc, and A49MI-VL-CL.

scFv-V3-46s-42-VL-VH-Fc (“Chain S”)
(SEQ ID NO: 204)
DIQMTQSPSSLSASVGDRVTITCRASEDISTAVAWYQQKPGKAPKLLIY
AASFLYSGVPSRFSGSGSGTDFTLTISSLQPEDFATYYCQQSQISPPTF
GCGTKVEIKGGGGSGGGGSGGGGSGGGGSEVQLVESGGGLVQPGGSLRL
SCAASGFTISSSSIHWVRQAPGKCLEWVAWVLPSVGFTDYADSVKGRFT
ISADTSKNTAYLQMNSLRAEDTAVYYCARRVCYNRLGVCAGGMDYWGQG
TLVTVSSASDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPRVYTLPPCRDEL
TKNQVSLTCLVKGFYPSDIAVEWESNGQPENNYKTTPPVLVSDGSFTLY
SKLTVDKSRWQQGNVFSCSVMHEALHNHYTQKSLSLSPG

scFv-V3-46s-42-VL-VH-Fc (SEQ ID NO:204) represents the full sequence of a BAFF-R binding scFv linked to an Fc domain via a hinge comprising Ala-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:141, which includes a heavy chain variable domain of V3-46s-42 connected to the C-terminus of a light chain variable domain of V3-46s-42 via a (G₄S)₄ linker. The scFv comprises substitution of Cys in the VH and VL regions at G44 and Q100, facilitating formation of a disulfide bridge between the VH and VL of the scFv.

A49MI-VH-CH1-Fc (SEQ ID NO:194) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-V3-46s-42-VL-VH-Fc. In A49MI-VH-CH1-Fc, the Fc domain also includes K360E and K409W substitutions for heterodimerization with the Fc in scFv-V3-46s-42-VL-VH-Fc.

A49MI-VL-CL (SEQ ID NO:195) represents the light chain portion of the Fab fragment comprising a light chain variable domain of NKG2D-binding A49MI (SEQ ID NO:85) and a light chain constant domain.

Another TriNKET described in the present disclosure is V3-46s-42-2-Fab. V3-46s-42-2-Fab includes (a) a BAFF-R-binding Fab fragment derived from V3-46s-42, including a heavy chain portion comprising a heavy chain variable domain and a CH1 domain, and a light chain portion comprising a light chain variable domain and a light chain constant domain, wherein the CH1 domain is connected to an Fc domain; (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. V3-46s-42-2-Fab includes four polypeptides: V3-46s-42-VH-CH1-Fc-Genmab, V3-46s-42-VL-CL, A49MI-VH-CH1-Fc-Genmab, and A49MI-VL-CL.

V3-46s-42-VH-CH1-Fc-Genmab
(SEQ ID NO: 202)
EVQLVESGGGLVQPGGSLRLSCAASGFTISSSSIHWVRQAPGKGLEWVA
WVLPSVGFTDYADSVKGRFTISADTSKNTAYLQMNSLRAEDTAVYYCAR
RVCYNRLGVCAGGMDYWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFLLYSKLTVDKSRWQQGNVFSCSVMHEALH
NHYTQKSLSLSPG
V3-46s-42-VL-CL
(SEQ ID NO: 206)
DIQMTQSPSSLSASVGDRVTITCRASEDISTAVAWYQQKPGKAPKLLIY
AASFLYSGVPSRFSGSGSGTDFTLTISSLQPEDFATYYCQQSQISPPTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

V3-46s-42-VH-CH1-Fc-Genmab (SEQ ID NO:205) represents the heavy chain portion of the Fab fragment, which comprises a heavy chain variable domain of BAFF-R-binding V3-46s-42 (SEQ ID NO:147) and a CH1 domain, connected to an Fc domain. The Fc domain in V3-46s-42-VH-CH1-Fc includes an F405L substitution for heterodimerization with the Fc in A49MI-VH-CH1-Fc-Genmab, which includes a K409R substitution.

V3-46s-42-VL-CL (SEQ ID NO:206) represents the light chain portion of the Fab fragment comprising a light chain variable domain of BAFF-R-binding V3-46s-42 (SEQ ID NO:150) and a light chain constant domain.

A49MI-VH-CH1-Fc-Genmab (SEQ ID NO:213) comprises a heavy chain variable domain of NKG2D-binding A49MI (SEQ ID NO:95) and a CH1 domain, connected to an Fc domain. The Fc domain in A49MI-VH-CH1-Fc-Genmab includes a K409R substitution for heterodimerization with the Fc in V3-46s-42-VH-CH1-Fc-Genmab, which includes an F405L substitution.

A49MI-VL-CL (SEQ ID NO:195) comprises a light chain variable domain of NKG2D-binding A49MI (SEQ ID NO:85) and a light chain constant domain.

Another exemplary TriNKET described in the present disclosure is Hu9.1-73-F3′ TriNKET. Hu9.1-73-F3′ TriNKET includes (a) a BAFF-R-binding scFv sequence derived from Hu9.1-73 of Table 2, in the orientation of VH positioned C-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. Hu9.1-73-F3′ includes three polypeptides: scFv-Hu9.1-73-VL-VH-Fc, A49MI-VH-CH1-Fc, and A49MI-VL-CL.

scFv-Hu9.1-73-VL-VH-Fc (“Chain S”)
(SEQ ID NO: 211)
DIQMTQSPSSLSASVGDRVTITCKSSQSLLYSSNQNNYLAWYQQKPGKA
PKLLIYWAQHLDSGVPSRFSGSGSGTDFTLTISSLQPEDFATYYCQQYY
TYPYTFGCGTKVEIKGGGGSGGGGSGGGGSGGGGSEVQLVESGGGLVQP
GGSLRLSCAASGLPMAGFYTSWVRQAPGKCLEWVGFIRDKANGYTTEYN
PSVKGRFTISADTSKNTAYLQMNSLRAEDTAVYYCAQVRRALDYWGQGT
LVTVSSASDKTHTCPPCPAPELLGGPSVFLFPPKPKDTLMISRTPEVTC
VVVDVSHEDPEVKFNWYVDGVEVHNAKTKPREEQYNSTYRVVSVLTVLH
QDWLNGKEYKCKVSNKALPAPIEKTISKAKGQPREPRVYTLPPCRDELT
KNQVSLTCLVKGFYPSDIAVEWESNGQPENNYKTTPPVLVSDGSFTLYS
KLTVDKSRWQQGNVFSCSVMHEALHNHYTQKSLSLSPG

scFv-Hu9.1-73-VL-VH-Fc (SEQ ID NO:211) represents the full sequence of a BAFF-R binding scFv linked to an Fc domain via a hinge comprising Ala-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:143, which includes a heavy chain variable domain of scFv-Hu9.1-73 connected to the C-terminus of a light chain variable domain of scFv-Hu9.1-73 via a (G₄S)₄ linker. The scFv comprises substitution of Cys in the VH and VL regions at G44 and Q100, facilitating formation of a disulfide bridge between the VH and VL of the scFv.

A49MI-VH-CH1-Fc (SEQ ID NO:194) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-Hu9.1-73-VL-VH-Fc. In A49MI-VH-CH1-Fc, the Fc domain also includes K360E and K409W substitutions for heterodimerization with the Fc in scFv-Hu9.1-73-VL-VH-Fc.

A49MI-VL-CL (SEQ ID NO:195) represents the light chain portion of the Fab fragment comprising a light chain variable domain of NKG2D-binding A49MI (SEQ ID NO:85) and a light chain constant domain.

Another TriNKET described in the present disclosure is Hu9.1-73-2-Fab. Hu9.1-73-2-Fab includes (a) a BAFF-R-binding Fab fragment derived from Hu9.1-73, including a heavy chain portion comprising a heavy chain variable domain and a CH1 domain, and a light chain portion comprising a light chain variable domain and a light chain constant domain, wherein the CH1 domain is connected to an Fc domain; (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. Hu9.1-73-2-Fab includes four polypeptides: Hu9.1-73-VH-CH1-Fc-Genmab, Hu9.1-73-VL-CL, A49MI-VH-CH1-Fc-Genmab, and A49MI-VL-CL.

Hu9.1-73-VH-CH1-Fc-Genmab
(SEQ ID NO: 212)
EVQLVESGGGLVQPGGSLRLSCAASGLPMAGFYTSWVRQAPGKGLEWVG
FIRDKANGYTTEYNPSVKGRFTISADTSKNTAYLQMNSLRAEDTAVYYC
AQVRRALDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFLLYSKLTVDKSRWQQGNVFSCSVMHEALHNHYTQKS
LSLSPG
Hu9.1-73-VL-CL
(SEQ ID NO: 205)
DIQMTQSPSSLSASVGDRVTITCKSSQSLLYSSNQNNYLAWYQQKPGKA
PKLLIYWAQHLDSGVPSRFSGSGSGTDFTLTISSLQPEDFATYYCQQYY
TYPYTFGQGTKVEIKRTVAAPSVFIFPPSDEQLKSGTASVVCLLNNFYP
REAKVQWKVDNALQSGNSQESVTEQDSKDSTYSLSSTLTLSKADYEKHK
VYACEVTHQGLSSPVTKSFNRGEC

Hu9.1-73-VH-CH1-Fc-Genmab (SEQ ID NO:212) represents the heavy chain portion of the Fab fragment, which comprises a heavy chain variable domain of BAFF-R-binding Hu9.1-73 (SEQ ID NO:151) and a CH1 domain, connected to an Fc domain. The Fc domain in Hu9.1-73-VH-CH1-Fc includes an F405L substitution for heterodimerization with the Fc in A49MI-VH-CH1-Fc-Genmab, which includes a K409R substitution.

Hu9.1-73-VL-CL (SEQ ID NO:205) represents the light chain portion of the Fab fragment comprising a light chain variable domain of BAFF-R-binding Hu9.1-73 (SEQ ID NO:152) and a light chain constant domain.

A49MI-VH-CH1-Fc-Genmab (SEQ ID NO:213) comprises a heavy chain variable domain of NKG2D-binding A49MI (SEQ ID NO:95) and a CH1 domain, connected to an Fc domain. The Fc domain in A49MI-VH-CH1-Fc-Genmab includes a K409R substitution for heterodimerization with the Fc in Hu9.1-73-VH-CH1-Fc-Genmab, which includes an F405L substitution.

A49MI-VL-CL (SEQ ID NO:195) comprises a light chain variable domain of NKG2D-binding A49MI (SEQ ID NO:85) and a light chain constant domain.

Another example of a TriNKET described in the present disclosure is AB1424/1612-F3′. AB1424/1612-F3′ includes (a) a BAFF-R-binding scFv sequence derived from AB1424/1612 (with cysteine heterodimerization mutations for disulfide bridge formation) of Table 2, in the orientation of VH positioned N-terminal to VL, linked to an Fc domain and (b) an NKG2D-binding Fab fragment derived from A49MI, including a heavy chain portion comprising a heavy chain variable domain and a CH1 domain, and a light chain portion comprising a light chain variable domain and a light chain constant domain, wherein the CH1 domain is connected to the Fc domain. AB1424/1612-F3′ includes three polypeptides: scFv-AB1424/1612-VL-VH-Fc (SEQ ID NO:193), A49MI-VH-CH1-Fc (SEQ ID NO:194), and A49MI-VL-CL (SEQ ID NO:195).

scFv-AB1424/1612-VH-VL-Fc (“Chain S”)
(SEQ ID NO: 270)
EVQLVQSGGGVVQPGRSLRLSCAASGFTFSSYGMHWVRQAPGKCLEWVA
VIWYDASNKYYGDSVKGRFTISRDNSKNTLYLQMNSLRDEDTAVYYCAR
RFTHLRGQYIEDYGLDVWGQGTTVTVSSGGGGSGGGGSGGGGSGGGGSE
IVLTQSPSSLSASVGDRVTITCRASQSISSYLNWYQQKPGKAPKLLIYA
ASSLQSGVPSRFSGSGSGTDFTLTISSLQPEDFATYYCQQSYSIPLTFG
CGTKVEIKGSDKTHTCPPCPAPELLGGPSVFLFPPKPKDTLMISRTPEV
TCVVVDVSHEDPEVKFNWYVDGVEVHNAKTKPREEQYNSTYRVVSVLTV
LHQDWLNGKEYKCKVSNKALPAPIEKTISKAKGQPREPRVYTLPPCRDE
LTKNQVSLTCLVKGFYPSDIAVEWESNGQPENNYKTTPPVLVSDGSFTL
YSKLTVDKSRWQQGNVFSCSVMHEALHNHYTQKSLSLSPG
A49MI-VH-CH1-Fc (“Chain H”)
(SEQ ID NO: 194)
EVQLVESGGGLVKPGGSLRLSCAASGFTFSSYSMNWVRQAPGKGLEWVS
SISSSSSYIYYADSVKGRFTISRDNAKNSLYLQMNSLRAEDTAVYYCAR
GAPIGAAAGWEDPWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVCTLPPSRDELTENQVSLTCLVKGFYPSDIAVEWESNGQP
ENNYKTTPPVLDSDGSFFLYSWLTVDKSRWQQGNVFSCSVMHEALHNHY
TQKSLSLSPG
A49MI-VL-CL (“Chain L”)
(SEQ ID NO: 195)
DIQMTQSPSSVSASVGDRVTITCRASQGISSWLAWYQQKPGKAPKLLIY
AASSLQSGVPSRFSGSGSGTDFTLTISSLQPEDFATYYCQQGVSFPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

scFv-AB1424/1612-VH-VL-Fc (SEQ ID NO:270) represents the full sequence of a BAFF-R binding scFv linked to an Fc domain via a hinge comprising Ala-Ser. The Fc domain linked to the scFv includes Q347R, D399V, and F405T substitutions for heterodimerization and an S354C substitution for forming a disulfide bond with a Y349C substitution in A49MI-VH-CH1-Fc as described below. The scFv has the amino acid sequence of SEQ ID NO:254, which includes a heavy chain variable domain of AB1424/1612 connected to the C-terminus of a light chain variable domain of AB1424/1612 via a (G₄S)₄ linker. The scFv comprises substitution of cysteine in the VH and VL regions at G44 and G100, facilitating formation of a disulfide bridge between the VH and VL of the scFv.

A49MI-VH-CH1-Fc (SEQ ID NO:194) represents the heavy chain portion of the Fab fragment, which comprises a heavy chain variable domain of NKG2D-binding A49MI (SEQ ID NO:95) and a CH1 domain, connected to an Fc domain. The Fc domain in A49MI-VH-CH1-Fc includes a Y349C substitution in the CH3 domain, which forms a disulfide bond with an S354C substitution on the Fc in scFv-AB1424/1612-VL-VH-Fc. In A49MI-VH-CH1-Fc, the Fc domain also includes K360E and K409W substitutions for heterodimerization with the Fc in scFv-AB1424/1612-VL-VH-Fc.

A49MI-VL-CL (SEQ ID NO:195) represents the light chain portion of the Fab fragment comprising a light chain variable domain of NKG2D-binding A49MI (SEQ ID NO:85) and a light chain constant domain.

Another example of a TriNKET described in the present disclosure is AB1424/1612-F4. AB1424/1612-F4 includes (a) two BAFF-R-binding Fab fragments derived from AB1424/1612 of Table 2, each including a heavy chain portion comprising a heavy chain variable domain and a CH1 domain, and a light chain portion comprising a light chain variable domain and a light chain constant domain, wherein the CH1 domain is connected to the Fc domain and (b) an NKG2D-binding scFv sequence derived from A49MI linked to the C-terminus of the Fc domain, in the orientation of VH positioned C-terminal to VL. AB1424/1612-F4 includes four polypeptides: a first polypeptide comprising AB1424/1612-VH-CH1-CH2-CH3-A49MI-scFv (SEQ ID NO:271), a second polypeptide comprising AB-1424/1612-VH-CH1-CH2-CH3 (SEQ ID NO:272), and a third and fourth polypeptide each comprising AB1424/1612-VL-CL (SEQ ID NO:273).

AB1424/1612-VH-CH1-CH2-CH3-A49MI-scFv (Chain “M”)
(SEQ ID NO: 271)
EVQLVQSGGGVVQPGRSLRLSCAASGFTFSSYGMHWVRQAPGKGLEWVA
VIWYDASNKYYGDSVKGRFTISRDNSKNTLYLQMNSLRDEDTAVYYCAR
RFTHLRGQYIEDYGLDVWGQGTTVTVSSASTKGPSVFPLAPSSKSTSGG
TAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPRVYTLPPCRDELTKNQVSLTCLVKGFYPSDIAVEWES
NGQPENNYKTTPPVLVSDGSFTLYSKLTVDKSRWQQGNVFSCSVMHEAL
HNHYTQKSLSLSPGSGSGGGGSDIQMTQSPSSVSASVGDRVTITCRASQ
GISSWLAWYQQKPGKAPKLLIYAASSLQSGVPSRFSGSGSGTDFTLTIS
SLQPEDFATYYCQQGVSFPRTFGCGTKVEIKGGGGSGGGGSGGGGSGGG
GSEVQLVESGGGLVKPGGSLRLSCAASGFTFSSYSMNWVRQAPGKCLEW
VSSISSSSSYIYYADSVKGRFTISRDNAKNSLYLQMNSLRAEDTAVYYC
ARGAPIGAAAGWFDPWGQGTLVTVSS
AB-1424/1612-VH-CH1-CH2-CH3 (Chain “H”)
(SEQ ID NO: 272)
EVQLVQSGGGVVQPGRSLRLSCAASGFTFSSYGMHWVRQAPGKGLEWVA
VIWYDASNKYYGDSVKGRFTISRDNSKNTLYLQMNSLRDEDTAVYYCAR
RFTHLRGQYIEDYGLDVWGQGTTVTVSSASTKGPSVFPLAPSSKSTSGG
TAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVCTLPPSRDELTENQVSLTCLVKGFYPSDIAVEWES
NGQPENNYKTTPPVLDSDGSFFLYSWLTVDKSRWQQGNVFSCSVMHEAL
HNHYTQKSLSLSPG
AB1424/1612-VL-CL (Chain “L”)
(SEQ ID NO: 273)
EIVLTQSPSSLSASVGDRVTITCRASQSISSYLNWYQQKPGKAPKLLIY
AASSLQSGVPSRFSGSGSGTDFTLTISSLQPEDFATYYCQQSYSIPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

AB1424/1612-VH-CH1-CH2-CH3-A49MI-scFv (SEQ ID NO:271) represents the heavy chain portion of the Fab fragment, which comprises a heavy chain variable domain of BAFF-R-binding AB1424/1612 (SEQ ID NO:250) and a CH1 domain, connected to an Fc domain, further connected to an scFv. The scFv has the amino acid sequence of SEQ ID NO:275, which comprises a heavy chain variable domain of NKG2D-binding A49MI (SEQ ID NO:95) connected to the C-terminus of a light chain variable domain of A49MI (SEQ ID NO:85) via a (G₄S)₄ linker. The scFv also comprises substitution of Cys in the VH and VL regions at G44 and G100, facilitating formation of a disulfide bridge between the VH and VL of the scFv. The scFv of AB1424/1612-VH-CH1-CH2-CH3-A49MI-scFv is linked to the C-terminus of the CH3 domain by a short SGSGGGGS (SEQ ID NO:274) linker. The Fc domain in AB1424/1612-VH-CH1-CH2-CH3-A49MI-scFv includes Q347R, D399V, and F405T substitutions for heterodimerization and an S354C substitution for forming a disulfide bond with a Y349C substitution in AB-1424/1612-VH-CH1-CH2-CH3 as described below.

AB1424/1612-VH-CH1-CH2-CH3 (SEQ ID NO:272) represents the heavy chain portion of the Fab fragment, which comprises a heavy chain variable domain of BAFF-R-binding AB1424/1612 (SEQ ID NO:250) and a CH1 domain, connected to an Fc domain. The Fc domain in A49MI-VH-CH1-Fc includes a Y349C substitution in the CH3 domain, which forms a disulfide bond with an S354C substitution on the Fc in AB1424/1612-VH-CH1-CH2-CH3-A49MI-scFv. In AB1424/1612-VH-CH1-CH2-CH3, the Fc domain also includes K360E and K409W substitutions for heterodimerization with the Fe in AB1424/1612-VH-CH1-CH2-CH3-A49MI-scFv.

AB1424/1612-VL-CL (SEQ ID NO:273) represents the light chain portion of the Fab fragment comprising a light chain variable domain of BAFF-R-binding AB1424/1612 (SEQ ID NO:251) and a light chain constant domain.

In certain embodiments, an F3′ TriNKET described in the present disclosure is identical to one of the exemplary TriNKETs described above, except that (a) the Fc domain linked to the NKG2D-binding Fab fragment includes Q347R, D399V, and F405T substitutions in the CH3 domain for heterodimerization, and the Fc domain linked to the BAFF-R-binding scFv includes matching K360E and K409W substitution in the CH3 domain; and/or (b) the Fc domain linked to the NKG2D-binding Fab fragment includes an S354C substitution in the CH3 domain, and the Fc domain linked to the BAFF-R-binding scFv includes a matching Y349C substitution in the CH3 domain for forming a disulfide bond.

In certain embodiments, a 2-Fab TriNKET described in the present disclosure is identical to one of the exemplary TriNKETs described above, except that the Fc domain linked to the NKG2D-binding Fab fragment includes a F405L substitution in the CH3 domain for heterodimerization, and the Fc domain linked to the BAFF-R-binding Fab fragment includes a matching K409R substitution in the CH3 domain.

A skilled person in the art would appreciate that during production and/or storage of proteins, N-terminal glutamate (E) or glutamine (Q) can be cyclized to form a lactam (e.g., spontaneously or catalyzed by an enzyme present during production and/or storage). Accordingly, in some embodiments where the N-terminal residue of an amino acid sequence of a polypeptide is E or Q, a corresponding amino acid sequence with the E or Q replaced with pyroglutamate is also contemplated herein.

A skilled person in the art would also appreciate that during protein production and/or storage, the C-terminal lysine (K) of a protein can be removed (e.g., spontaneously or catalyzed by an enzyme present during production and/or storage). Such removal of K is often observed with proteins that comprise an Fc domain at its C-terminus. Accordingly, in some embodiments where the C-terminal residue of an amino acid sequence of a polypeptide (e.g., an Fc domain sequence) is K, a corresponding amino acid sequence with the K removed is also contemplated herein.

The multispecific proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins.

To achieve the highest yield of the multispecific protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multispecific protein. The multispecific proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### II. Characteristics of the Multispecific Proteins

The multispecific proteins described herein include an NKG2D-binding site, a BAFF-R binding site, and an antibody Fc domain or a portion thereof sufficient to bind CD16, or an antigen-binding site that binds CD16. In some embodiments, the multispecific proteins contains an additional antigen-binding site that binds BAFF-R, as exemplified in the F4-TriNKET format (e.g., FIGS. 2C and 2D).

In some embodiments, the multispecific proteins display similar thermal stability to the corresponding monoclonal antibody, i.e., a monoclonal antibody containing the same BAFF-R binding site as the one incorporated in the multispecific proteins.

In some embodiments, the multispecific proteins simultaneously bind to cells expressing NKG2D and/or CD16, such as NK cells, and cells expressing BAFF-R, such as certain tumor cells. Binding of the multispecific proteins to NK cells can enhance the activity of the NK cells toward destruction of the BAFF-R expressing cells (e.g., BAFF-R expressing tumor cells). It has been reported that NK cells exhibit more potent cytotoxicity against target cells that are stressed (see Chan et al., (2014) Cell Death Differ. 21(1):5-14). Without wishing to be bound by theory, it is hypothesized that when NK cells are engaged to a population of cells by a TriNKET, the NK cells may selectively kill the target cells that are stressed (e.g., malignant cells and cells in a tumor microenvironment). This mechanism could contribute to increased specificity and reduced toxicity of TriNKETs, making it possible to selectively clear the stressed cells even if expression of BAFF-R is not limited to the desired target cells.

In some embodiments, the multispecific proteins bind to BAFF-R with a similar affinity to the corresponding the anti-BAFF-R monoclonal antibody (i.e., a monoclonal antibody containing the same BAFF-R binding site as the one incorporated in the multispecific proteins). In some embodiments, the multispecific proteins are more effective in killing the tumor cells expressing BAFF-R than the corresponding monoclonal antibodies.

In certain embodiments, the multispecific proteins described herein, which include a binding site for BAFF-R, activate primary human NK cells when co-culturing with cells expressing BAFF-R. NK cell activation is marked by the increase in CD107a degranulation and IFN-7 cytokine production. Furthermore, compared to a corresponding anti-BAFF-R monoclonal antibody, the multispecific proteins can show superior activation of human NK cells in the presence of cells expressing BAFF-R.

In some embodiments, the multispecific proteins described herein, which include a binding site for BAFF-R, enhance the activity of rested and IL-2-activated human NK cells when co-culturing with cells expressing BAFF-R.

In some embodiments, compared to the corresponding monoclonal antibody that binds to BAFF-R, the multispecific proteins offer an advantage in targeting tumor cells that express medium and low levels of BAFF-R.

In some embodiments, the bivalent F4 format of the TriNKETs (i.e., TriNKETs include an additional antigen-binding site that binds to BAFF-R) improve the avidity with which the TriNKETs bind to BAFF-R, which in effect stabilizes expression and maintenance of high levels of BAFF-R on the surface of the tumor cells. In some embodiments, the F4-TriNKETs mediate more potent killing of tumor cells than the corresponding F3-TriNKETs or F3′-TriNKETs.

### III. Therapeutic Applications

The present application also describes methods for treating autoimmune disease or cancer using a multispecific binding protein described herein and/or a pharmaceutical composition described herein. The methods may be used to treat a variety of cancers or autoimmune diseases expressing BAFF-R.

The therapeutic method can be characterized according to the cancer to be treated. The cancer to be treated can be characterized according to the presence of a particular antigen expressed on the surface of the cancer cell, e.g., BAFF-R.

Cancers characterized by the expression of BAFF-R, include, without limitation, B-cell non-Hodgkin's lymphoma (B-NHL), such as chronic lymphocytic leukemia (CLL), mantle cell lymphoma (MCL), follicular lymphoma (FL), diffuse large B-cell lymphoma (DLBCL), marginal zone lymphoma, mucosa-associated lymphoid tissue (MALT) lymphoma, primary mediastinal B-cell lymphoma, acute lymphocytic leukemia (ALL); and autoimmune inflammatory diseases.

It is contemplated that the protein, conjugate, cells, and/or pharmaceutical compositions described in the present disclosure can be used to treat a variety of cancers, not limited to cancers in which the cancer cells or the cells in the cancer microenvironment express BAFF-R.

In certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain embodiments, the cancer is a hematologic malignancy. In certain embodiments, the hematologic malignancy is leukemia. In certain embodiments, selected from the group consisting of acute myeloid leukemia (AML), acute lymphoblastic leukemia (ALL), myelodysplasia, myelodysplastic syndromes, acute T-lymphoblastic leukemia, or acute promyelocytic leukemia, chronic myelomonocytic leukemia, or myeloid blast crisis of chronic myeloid leukemia.

In some embodiments, the present application provides methods for treating an autoimmune inflammatory disease using a multispecific binding protein described herein and/or a pharmaceutical composition described herein. The methods may be used to treat a variety of BAFF-R-expressing B cell-associated autoimmune inflammatory diseases, including, without limitation, multiple sclerosis, systemic lupus erythematosus, Graves' disease, Hashimoto's thyroiditis, rheumatoid arthritis, inflammatory bowel disease, type I diabetes, Guillain-Barre syndrome, chronic inflammatory demyelinating polyneuropathy, psoriasis, myasthenia gravis, and vasculitis.

### IV. Combination Therapy

Another aspect of the present application provides for combination therapy. A multispecific binding protein described herein can be used in combination with additional therapeutic agents to treat autoimmune disease or to treat cancer.

Exemplary therapeutic agents that may be used as part of a combination therapy in treating autoimmune inflammatory diseases are described in Li et al. (2017) Front. Pharmacol., 8:460, and include, for example, non-steroidal anti-inflammatory drugs (NSAIDs) (e.g., COX-2 inhibitors), glucocorticoids (e.g., prednisone/prednisolone, methylprednisolone, and the fluorinated glucocorticoids such as dexamethasone and betamethasone), disease-modifying antirheumatic drugs (DMARDs) (e.g., methotrexate, leflunomide, gold compounds, sulfasalazine, azathioprine, cyclophosphamide, antimalarials, D-penicillamine, and cyclosporine), anti-TNF biologics (e.g., infliximab, etanercept, adalimumab, golimumab, Certolizumab pegol, and their biosimilars), and other biologics targeting CTLA-4 (e.g., abatacept), IL-6 receptor (e.g., tocilizumab), IL-1 (e.g., anakinra), Th1 immune responses (IL-12/IL-23) (e.g., ustekinumab), Th17 immune responses (IL-17) (e.g., secukinumab) and CD20 (e.g., rituximab).

Exemplary therapeutic agents that may be used as part of a combination therapy in treating cancer include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-7), colony stimulating factor-1, colony stimulating factor-2, denileukin diftitox, interleukin-2, luteinizing hormone releasing factor and variations of the aforementioned agents that may exhibit differential binding to its cognate receptor, or increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the present application can also be used as an adjunct to surgical removal of the primary lesion.

The amount of multispecific binding protein and additional therapeutic agent, and the relative timing of administration, may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multispecific binding protein may be administered during a time when the additional therapeutic agent(s) exerts its prophylactic or therapeutic effect, or vice versa.

### V. Pharmaceutical Compositions

The present disclosure also describes pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The intravenous drug delivery formulation described in the present application may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may be freeze-dried (lyophilized) and contained in about 12-60 vials. In certain embodiments, the formulation may be freeze-dried and 45 mg of the freeze-dried formulation may be contained in one vial. In certain embodiments, the about 40 mg to about 100 mg of freeze-dried formulation may be contained in one vial. In certain embodiments, freeze-dried formulation from 12, 27, or 45 vials are combined to obtain a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

The protein could exist in a liquid aqueous pharmaceutical formulation including a therapeutically effective amount of the protein in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, for example between 5 and 9 or between 6 and 8, and in certain embodiments, between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present application describes a formulation with an extended shelf life including a multispecific binding protein as described herein, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including a protein of the present disclosure in a pH-buffered solution. The buffer of the formulation may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86 mg/mL), and about 6.2 mg/mL of sodium chloride (e.g., 6.165 mg/mL). In certain embodiments, the buffer system includes about 1 to about 1.5 mg/mL of citric acid, about 0.25 to about 0.5 mg/mL of sodium citrate, about 1.25 to about 1.75 mg/mL of disodium phosphate dihydrate, about 0.7 to about 1.1 mg/mL of sodium dihydrogen phosphate dihydrate, and about 6.0 to about 6.4 mg/mL of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to about 15 mg/mL. In certain embodiments, the concentration of mannitol may be about 10 to about 14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, a multispecific binding protein as described in the present application is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with 61.2 mL of the protein product solution in order to allow an extractable volume of 60 mL. In certain embodiments, the liquid formulation may be diluted with 0.9% saline solution.

In certain embodiments, the liquid formulation as described in this application may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be a disaccharide, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation as described in this application may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations described herein to reduce bacterial action. The addition of a preservative may, for example, facilitate the production of a multi-use (multiple-dose) formulation.

Intravenous (IV) formulations may be an administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A multispecific binding protein as described in the present application could exist in a lyophilized formulation including the proteins and a lyoprotectant. The lyoprotectant may be a sugar, e.g., a disaccharide. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing a protein of the present disclosure may be adjusted between 6 to 8. In certain embodiments, the pH range for the lyophilized drug product may be from 7 to 8.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the multispecific binding proteins described in the present application may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution.

In certain embodiments, the lyophilized drug product is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product is constituted to about 4.5 mL water for injection and diluted with 0.9% saline solution (sodium chloride solution).

Actual dosage levels of the active ingredients in the pharmaceutical compositions of multispecific binding proteins described in this application may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 g to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about g to about 100 mg/kg of body weight, about 10 μg to about 50 mg/kg of body weight, about g to about 10 mg/kg of body weight, about 10 μg to about 1 mg/kg of body weight, about 10 g to about 100 μg/kg of body weight, about 10 μg to about 50 μg/kg of body weight, about 50 g to about 100 mg/kg of body weight, about 50 μg to about 50 mg/kg of body weight, about 50 g to about 10 mg/kg of body weight, about 50 μg to about 1 mg/kg of body weight, about 50 μg to about 100 μg/kg of body weight, about 100 μg to about 100 mg/kg of body weight, about 100 g to about 50 mg/kg of body weight, about 100 μg to about 10 mg/kg of body weight, about 100 g to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the multispecific binding proteins described in the present application could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above provides multiple aspects and embodiments of the multispecific binding proteins described in the application. The patent application specifically contemplates all combinations and permutations of the aspects and embodiments. The use of any and all examples, or exemplary language herein, for example, “such as” or “including,” is intended merely to illustrate better the multispecific binding proteins described in the present application, and does not pose a limitation on the scope of the disclosure, unless so expressly stated. No language in the specification should be construed as indicating any non-claimed element as essential to the practice of the multispecific binding proteins described in the present application.

### EXAMPLES

The following examples are merely illustrative and are not intended to limit the scope or content of the multispecific binding proteins described in the present application in any way.

### Example 1—Assessment of TriNKET Binding to Cell Expressed Human BAFF-R

The BAFF-R positive human B lymphoblastoid RAJI cell line was used to assess TriNKET binding to cell surface BAFF-R. Certain BAFF-R TriNKETs in the 2-Fab and F3′ formats, as described in the “exemplary multispecific binding proteins” subsection above, were diluted and incubated with Raji cells. Binding patterns of TriNKETs and parental monoclonal antibodies were detected using a fluorophore conjugated anti-human IgG secondary antibody. The cells were then incubated with a fluorophore conjugated anti-human IgG secondary antibody and were analyzed by flow cytometry. The mean fluorescence intensity (MFI) values were normalized to secondary antibody only controls to obtain fold over background (FOB) values.

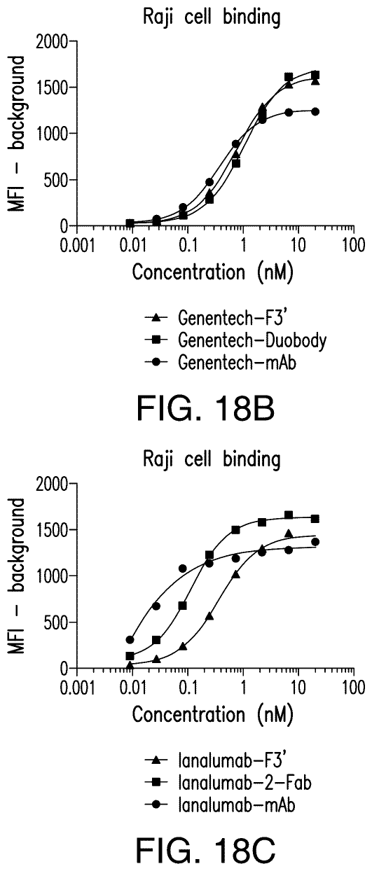

As shown in FIG. 18A-FIG. 18C, BAFF-R TriNKETs containing a BAFF-R binding site derived from hCOH-2 (FIG. 18A), Hu9.1-73 (FIG. 18B), and ianalumab-based antigen-binding site (the three versions, F3′, 2-Fab, and ianalumab-mAb, do not contain antibody-dependent cellular cytotoxicity-enhancing mutations present in the commercial ianalumab antibody) (FIG. 18C) bind with subnanomolar concentration and with similar or higher maximum MFI than the corresponding parental control antibodies, which does not contain ADCC-enhancing mutations used in ianalumab. The EC₅₀ values of these TriNKETs binding BAFF-R are shown in Table 11. Similar results were obtained with a second BAFF-R positive cell line, Ramos (data not shown).

TABLE 11
EC₅₀ values in BAFF-R binding assay using RAJI cells

| Test article* | EC50 (nM) |
|---|---|
| Ianalumab-F3' | 0.361 |
| Ianalumab-2-Fab | 0.115 |
| Ianalumab-mAb | 0.003 |
| Hu9.1-73-F3' | 0.765 |
| Hu9.1-73-2-Fab | 1.054 |
| Hu9.1-73-mAb | 0.379 |
| hCOH-2-F3' | 0.893 |
| hCOH-2-2-Fab | 0.942 |
| hCOH-2-mAb | 0.043 |

*The ianalumab constructs do not include antibody-dependent cellular cytotoxicity-enhancing mutations present in the commercial ianalumab antibody.

### Example 2—Human NK Cell Cytotoxicity Assay

Lysis of BAFF-R-expressing target cells by immune effector cells in the presence of the TriNKETs was measured by the DELFIA cytotoxicity assay. Briefly, human cancer cell line RAJI expressing BAFF-R was harvested from culture, washed with HBS, and resuspended in growth media at 10⁶/mL for labeling with BATDA reagent (Perkin Elmer AD0116). Manufacturer instructions were followed for labeling of the target cells. After labeling, cells were washed three times with HBS, and were resuspended at 0.5-1.0×10⁵/mL in culture media. 100 μl of BATDA labeled cells were added to each well of the 96-well plate. Monoclonal antibodies or TriNKETs against BAFF-R were diluted in culture media, and 50 μl of diluted mAb or TriNKET were added to each well.

To prepare NK cells, PBMCs were isolated from human peripheral blood buffy coats using density gradient centrifugation, washed, and prepared for NK cell isolation. NK cells were isolated using a negative selection technique with magnetic beads. Purity of isolated NK cells was typically >90% CD3⁻CD56⁺. Isolated NK cells were rested overnight and harvested from culture. The cells were then washed and resuspended at concentrations of 10⁵-2.0×10⁶/mL in culture media for an effector-to-target (E:T) ratio of 5:1. 50 μl of NK cells were added to each well of the plate for a total of 200 μl culture volume. The plate was incubated at 37° C. with 5% CO₂ for 2-3 hours.

After the incubation, the plate was removed from the incubator and the cells were pelleted by centrifugation at 200×g for 5 minutes. 20 μl of culture supernatant were transferred to a clean microplate and 200 μl of room temperature europium solution (Perkin Elmer C135-100) were added to each well. The plate was protected from light and incubated on a plate shaker at 250 rpm for 15 minutes, then read using SpectraMax i3X instruments.

Spontaneous release of substance that can form a fluorescent chelate with europium was measured in target cells incubated in the absence of NK cells. Maximum release of such substance was measured in target cells lysed with 1% Triton-X. % Specific lysis was calculated as follows:

$$\% Specific lysis = ( ( Experimental release - Spontaneous release ) /$$

$$( Maximum release - Spontaneous release ) ) \times 100 \% .$$

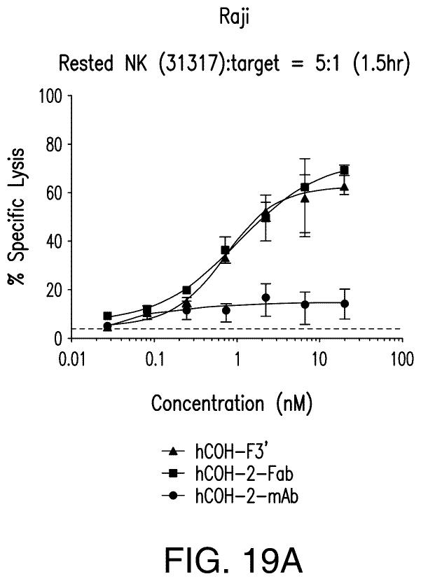

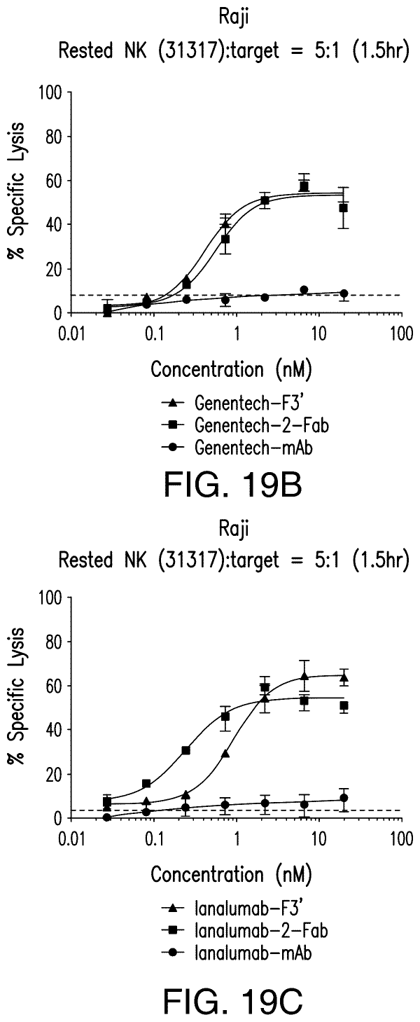

FIG. 19A-FIG. 19C show NK cell-mediated lysis of BAFF-R-positive RAJI cells by primary NK cells in the presence of BAFF-R-targeting TriNKETs derived from hCOH-2 (FIG. 19A), Genentech Hu9.1-73 (FIG. 19B), and ianalumab-based antigen-binding site (the three versions, F3′, 2-Fab, and ianalumab-mAb, do not contain antibody-dependent cellular cytotoxicity-enhancing mutations present in the commercial ianalumab antibody) (FIG. 19C). Parental BAFF-R targeted monoclonal antibodies showed little enhancement of NK cell mediated lysis of RAJI target cells. All BAFF-R targeted TriNKETs (hCOH-2-F3′, hCOH-2-2-Fab, Hu9.1-73-F3′, Hu9.1-73-2-Fab, ianalumab-F3′, and ianalumab-2-Fab) showed superior lysis of target cells compared to the respective BAFF-R targeted monoclonal antibody. EC₅₀ values are shown in Table 12.

TABLE 12
Potency of BAFF-R TriNKETs in comparison with parental
mAb in primary NK mediated cytotoxicity assay.

| Test article* | EC50 (nM) | Max lysis (%) |
|---|---|---|
| Ianalumab-F3' | 0.935 | 64.83 |
| Ianalumab-2-Fab | 0.244 | 54.30 |
| Ianalumab-mAb | N/A | N/A |
| Hu9.1-73-F3' | 0.417 | 54.16 |
| Hu9.1-73-2-Fab | 0.559 | 53.18 |
| Hu9.1-73-mAb | N/A | N/A |
| hCOH-2-F3' | 0.725 | 62.59 |
| hCOH-2-2-Fab | 1.042 | 73.53 |
| hCOH-2-mAb | N/A | N/A |

*Ianalumab constructs do not include antibody-dependent cellular cytotoxicity-enhancing mutations present in the commercial ianalumab antibody.

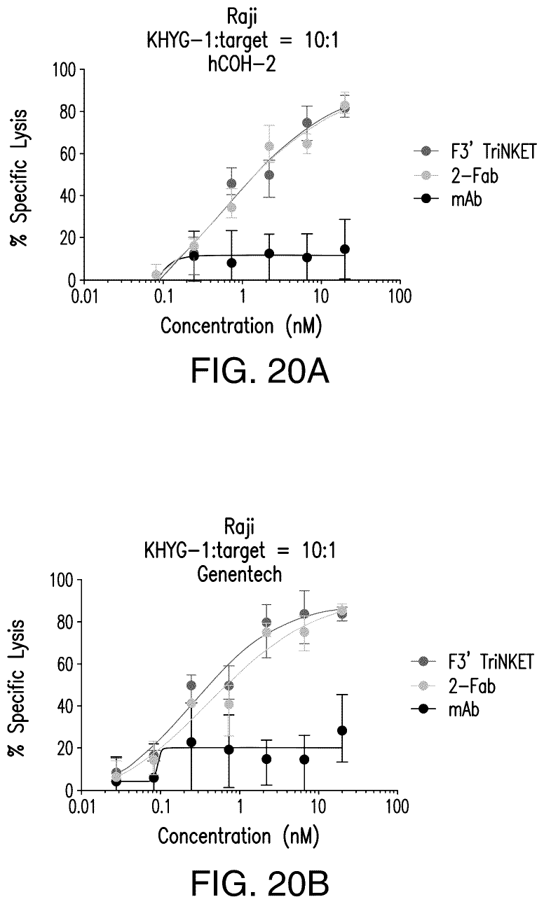

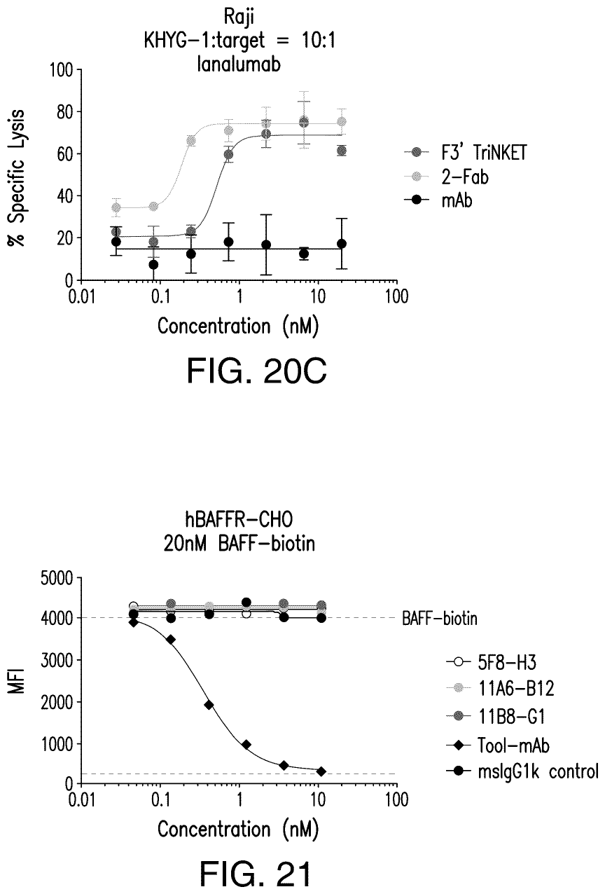

To confirm the cytoxicity findings, an NK cell line, KHYG-1-CD16aV was engineered to stably express CD16aV and NKG2D, and the assay was conducted as described above. Cytotoxicity was measured for TriNKETs and parental mAbs derived from hCOH-2 (FIG. 20A), Hu9.1-73 (FIG. 20B) and ianalumab-based antigen-binding site (the three versions, F3′, 2-Fab, and ianalumab-mAb, do not contain antibody-dependent cellular cytotoxicity-enhancing mutations present in the commercial ianalumab antibody) (FIG. 20C). The EC₅₀ values and maximum lysis values were derived from the cell lysis curves by the GraphPad Prizm software using four parameter logistic non-linear regression curve fitting model (Table 13). All BAFF-R TriNKETs tested showed subnanomolar EC50 and high efficiency maximum lysis in the KHYG-1 CD16aV mediated cytotoxicity assay. Similar results were obtained with a second BAFF-R positive cell line, Ramos (data not shown).

TABLE 13
Potency of BAFF-R TriNKETs in comparison with parental
mAb in primary KHYG-1 CD16aV mediated cytotoxicity assay.

| Test article* | EC50 (nM) | Max lysis (%) |
|---|---|---|
| Ianalumab-F3' | 0.515 | 68.35 |
| Ianalumab-2-Fab | 0.183 | 74.02 |
| Ianalumab-mAb | N/A | 14.48 |
| Hu9.1-73-F3' | 0.246 | 89.11 |
| Hu9.1-73-2-Fab | 0.402 | 92.94 |
| Hu9.1-73-mAb | N/A | 19.84 |
| hCOH-2-F3' | 0.448 | 97.52 |
| hCOH-2-2-Fab | 0.539 | 93.76 |
| hCOH-2-mAb | N/A | 11.56 |

*Ianalumab constructs do not include antibody-dependent cellular cytotoxicity-enhancing mutations present in the commercial ianalumab antibody.

### Example 3—Generation and Characterization of BAFF-R Binding mAbs

### Recombinant Protein Immunization Methods

BAFF-R-specific antibodies were generated by immunizing four different strains of mice (H2L2, NZBW, BALB-C, and SJL/J) with hBAFF-R-hFc-His fusion protein. Based on antisera titers, a total of seven mice from across the four different strains were selected for hybridoma fusion. Splenocytes from a subset of mice from each immunization arm were reserved for immune library generation; however, only splenocytes from H2L2 mice were used for yeast display mAb discovery.

From five mice fusions (splenocytes from two mice were pooled for H2L2 fusion and splenocytes from two mice were pooled for SJL/J fusion), sixteen 96-well plates per hybridoma fusion were analyzed by specificity ELISA, in which binding to human and cynomolgus monkey BAFF-R-hFc-His and binding to irrelevant-hFc-His protein was compared. Supernatants from 33 BAFF-R positive and specific hybridomas were selected for further analysis. Supernatants were tested for binding to BAFF-R+ isogenic CHO cells, and 16 positive hybridomas were further subcloned. Supernatants from the subclones were analyzed by specificity ELISA as described above and 20 BAFF-R positive and specific subclones were tested for binding to BAFF-R+ cells. Nine subclone mAbs demonstrated strong binding to BAFF-R+ cells and were sequenced. Six unique sequences were obtained, and the corresponding mAbs were further analyzed for their ability to block BAFF-R-BAFF interactions in a cell-based assay.

Binding of biotinylated BAFF to BAFF-R+ CHO cells was tested in the absence or presence of the six BAFF-R specific mAbs or an isotype control mAb. Reduction of mean fluorescent intensity (MFI) in the presence of antibody suggested that a mAb inhibited engagement of BAFF binding to BAFF-R, thus was designated as blocking antibody. All clones tested did not inhibit BAFF binding to BAFF-R+ cells, and therefore, all six were termed non-blocking (FIG. 21).

### DNA Immunization Methods

DNA immunization of two groups of SWR/J mice each was performed. One group was immunized with a full-length human BAFF-R cDNA construct, and the other with a mixture of full-length human BAFF-R and human BAFF-R extracellular domain cDNA constructs. Based on antisera titers, mice were pooled, and subsequently selected for single B cell sorting and another pool used for hybridoma fusion.

Single B cell sorting efforts yielded 44 human and cynomolgus monkey cross-reactive clones. These clones were sequenced, transiently expressed in 293 cells, and the specificity of the purified mAb was analyzed by flow cytometry in which binding to hBAFF-R⁺, cynoBAFF-R⁺ isogenic CHO cells and to parental cell line was compared. Eight binders were purified and further analyzed for their ability to bind to BAFF-R and to block BAFF-R-BAFF interactions. All eight clones were determined non-blocking and demonstrated weak affinity for hBAFF-R⁺ cancer cells.

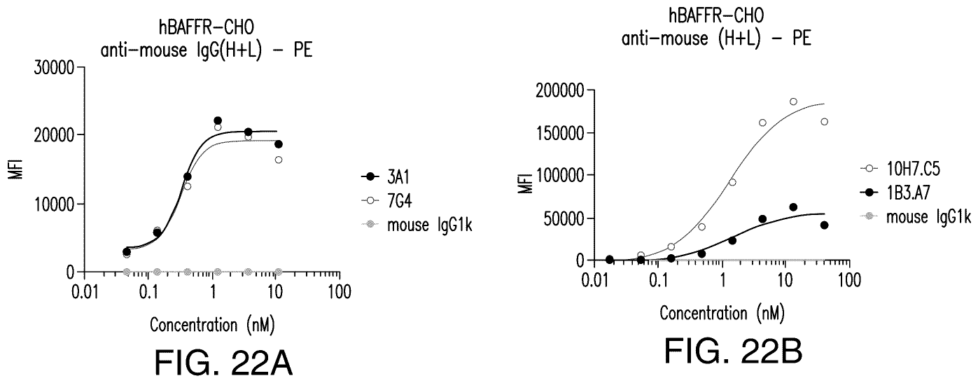

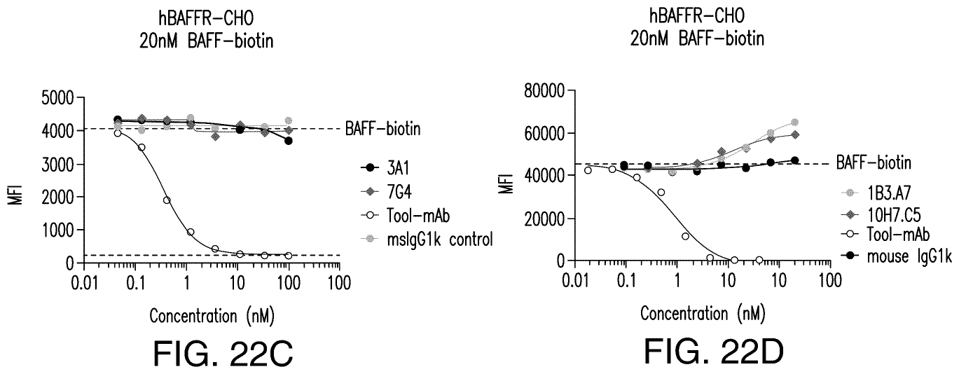

Specificity of clones obtained by the traditional hybridoma approach analyzed by flow cytometry. The following assessment was performed: a) binding to cells expressing either full-length human BAFF-R or human BAFF-R extracellular domain was compared to binding to non-transfected parental cells; b) binding to hBAFF-R⁺ and cynoBAFF-R⁺ isogenic cell was compared to binding to the parental cells; c) binding to hBAFF-R⁺ cancer cells. 25 positive hybridoma fusions were identified and based on the binding intensity 14 hybridoma fusions were sequenced. Five unique sequences were obtained and analyzed for their ability to bind BAFF-R⁺ cells and block BAFF-R-BAFF interactions. Though all five clones were determined non-blocking clones (FIG. 22), four out of five clones (clones 3A1, 1B3-A7, 7G4 and 10H7-C5) demonstrated good affinity for hBAFF-R.

BAFF-R Specific scFvs Discovered from Yeast Libraries

Yeast display was used to build scFv libraries from the splenocytes obtained from humanized H2L2 mice immunized with recombinant human hBAFF-R-hFc-His protein as described above. Three rounds of selection were carried out with biotinylated hBAFF-R-hFc-His at 5 nM. Individual yeast colonies were picked, sequenced, and sequences analyzed. Sequence convergence indicated the selection process was successful in enriching for binders and was therefore complete. Unique sequences were selected for further characterization. Three BAFF-R specific scFvs were discovered from one library (Table 14). However, these sequences were very similar to each other, and therefore only sequence 1129_A01 (also referred to as AB0369scFv) was selected for further study.

TABLE 14
CDR sequences of BAFF-R binders discovered from yeast library

| | 1129_A01 (AB0369 scFv) | 1203_A01 | 1203_A02 |
|---|---|---|---|
| CDRH1 | GFTFSSY | GFTFSSY | GFTFSTY |
| | (SEQ ID NO: 214) | (SEQ ID NO: 214) | (SEQ ID NO: 220) |
| CDRH2 | WYDGSN | WYDGSN | WYDGSN |
| | (SEQ ID NO: 215) | (SEQ ID NO: 215) | (SEQ ID NO: 215) |
| CDRH3 | RFTMLRGLIIEDYGMDV | RFTMLRGVFIEDYGMDV | RNTMVRGVIIEDYGMDV |
| | (SEQ ID NO: 216) | (SEQ ID NO: 219) | (SEQ ID NO: 221) |
| CDRL1 | RASQSISSYLN | RASQSVSSNLA | RASQSISSYLN |
| | (SEQ ID NO: 217) | (SEQ ID NO: 59) | (SEQ ID NO: 217) |
| CDRL2 | AASSLQS | GASTRAT | AASSLQS |
| | (SEQ ID NO: 77) | (SEQ ID NO: 60) | (SEQ ID NO: 77) |
| CDRL3 | QQSYSTPLT | QQSYSTPLT | QQSYSSPLT |
| | (SEQ ID NO: 218) | (SEQ ID NO: 218) | (SEQ ID NO: 222) |

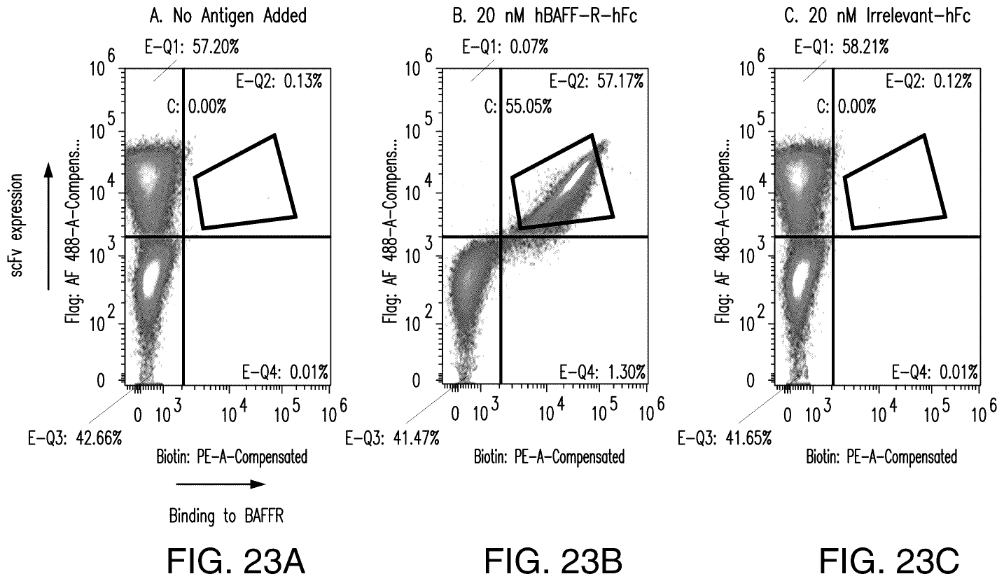

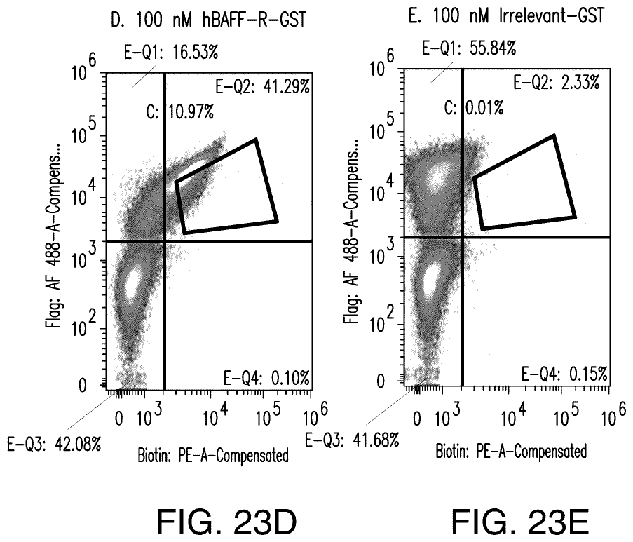

Flow cytometry was used to assess specificity of binding of AB0369scFv to hBAFF-R-hFc-His, hBAFF-R-GST-His, and negative control proteins with hFc tag or GST tag while displayed on yeast. AB0369scFv demonstrated medium to weak affinity towards hBAFF-R; however, it did not show binding to the negative control, thus suggesting high specificity for BAFF-R (FIG. 23).

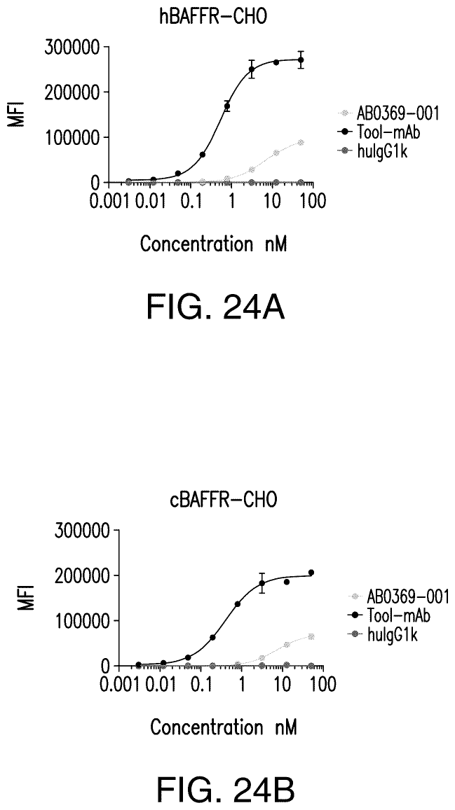

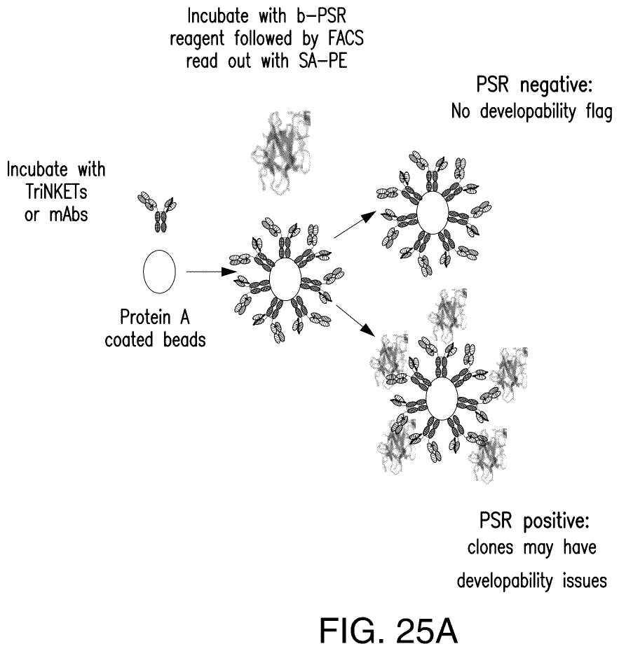

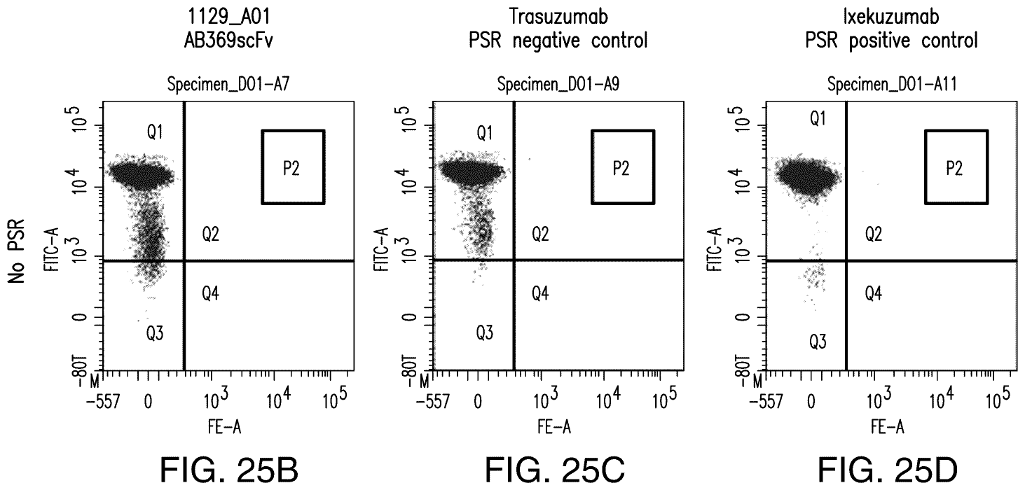

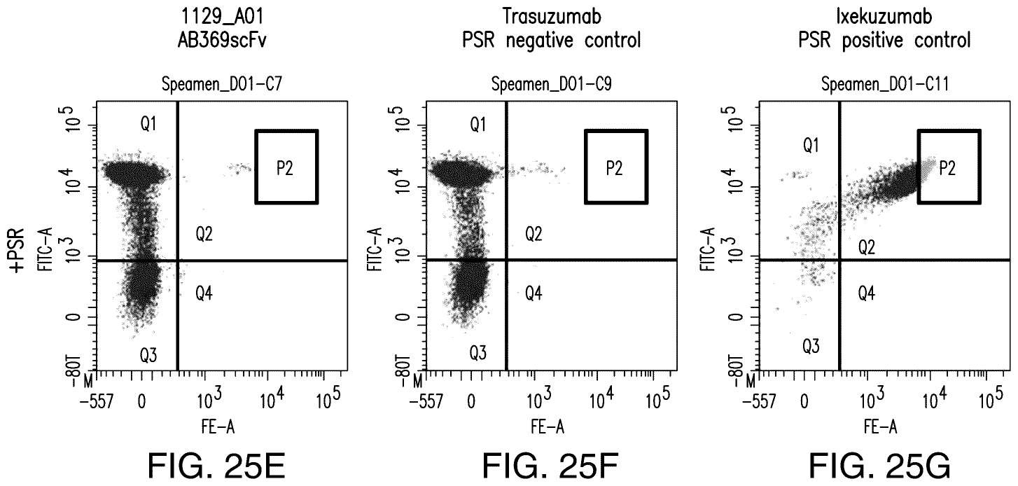

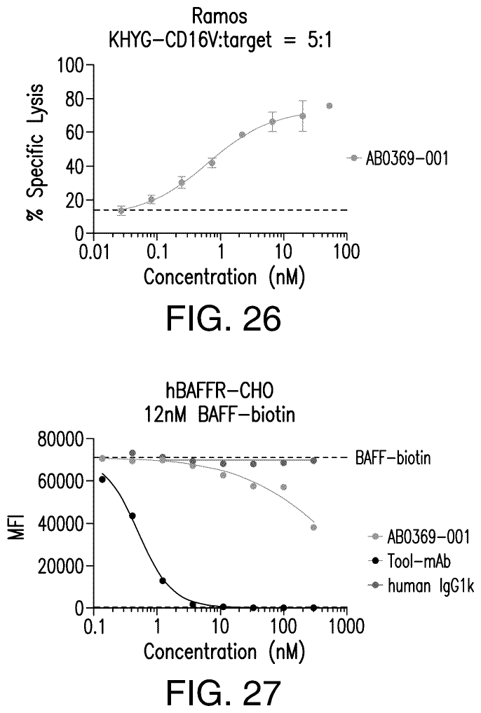

1129_A01 (AB0369 scFv) was converted into a multispecific binding protein comprising the scFv, and two non-BAFF-R binders, to yield AB0369. AB0369 was further analyzed for its abilities to bind to human (hBAFF-R-CHO) and cynomolgus monkey (cBAFF-R-CHO) BAFF-R⁺ cells (FIG. 24A, FIG. 24B), lack non-specific interactions by polyspecificity reagent (PSR) assay (FIG. 25A-FIG. 25G), lyse BAFF-R⁺ Ramos cancer cells (FIG. 26 and Table 15) and block BAFF-BAFF-R interactions (FIG. 27). AB0369 bound to both human and cynomolgus monkey BAFF-R on the surface of isogenic CHO cells, and BAFF-R binding was with EC₅₀ about 10 nM, making it a good choice for further development.

TABLE 15
Potency of AB0369 in KHYG-1-CD16aV cytotoxicity assay.

| Molecule | EC₅₀ (nM) | Max Lysis (%) |
|---|---|---|
| AB0369-001 | 0.6 | 73 |

The ability of AB0369 to block BAFF-R-BAFF interactions was tested in a cell-based blocking assay. Briefly, CHO cells expressing human BAFF-R were harvested, washed in cold FACS buffer, and seeded at a density of 100,000 cells per well. Test articles were diluted in FACS buffer, and 50 μL of diluted multispecific binding protein or mAb was added to cells, incubated on ice for 60 minutes, then washed with FACS buffer. 12 nM BAFF-biotin was diluted into FACS buffer, and 100 μL was added per well, incubated for 60 minutes on ice, then washed with FACS buffer. Cells were incubated with 100 μL of 1:200 streptavidin-PE diluted in FACS buffer and incubated on ice for 30 minutes then washed with FACS buffer. Cells were then incubated in 100 μL of 1:1,000 dilution of live/dead dye in PBS for 15 minutes, then washed with FACS buffer, and fixed. After incubation, cells were washed with FACS buffer and resuspended in FACS buffer for analysis with flow cytometry. Median fluorescent intensity (MFI) of each sample and the secondary-only control was calculated. Maximum MFI was calculated as BAFF-biotin alone, and minimum MFI was calculated as streptavidin-Phycoerythrin alone. Data were fit to a four-parameter non-linear regression curve using GraphPad Prism.

These studies revealed that AB0369 was able to partially block BAFF-R-BAFF interactions. However, the blocking was significantly less potent than the ianalumab-based benchmark control, which does not contain antibody-dependent cellular cytotoxicity enhancing mutations unlike the parent antibody, presumably due to the low affinity of AB0369 (FIG. 27 and Table 16). As the AB0369 scFv was the only blocking antibody identified from all discovery efforts described above, it underwent further development by affinity maturation of CDRH3 and CDRH1/CDRH2, as well as further amino acid changes to facilitate protein production and stability.

TABLE 16
Summary of AB0369 and benchmark mAb blocking of BAFF
binding to cellular BAFF-R.

| Molecule | IC₅₀ (nM) | Minimum (MFI) |
|---|---|---|
| AB0369-001 | 488 | 38,180 |
| Ianalumab-based tool-mAb | 0.5 | 224 |
| Human IgG1k | N/A | 68,050 |

### Affinity Maturation of AB0369

### CDRH3 Focused Randomized Affinity Maturation

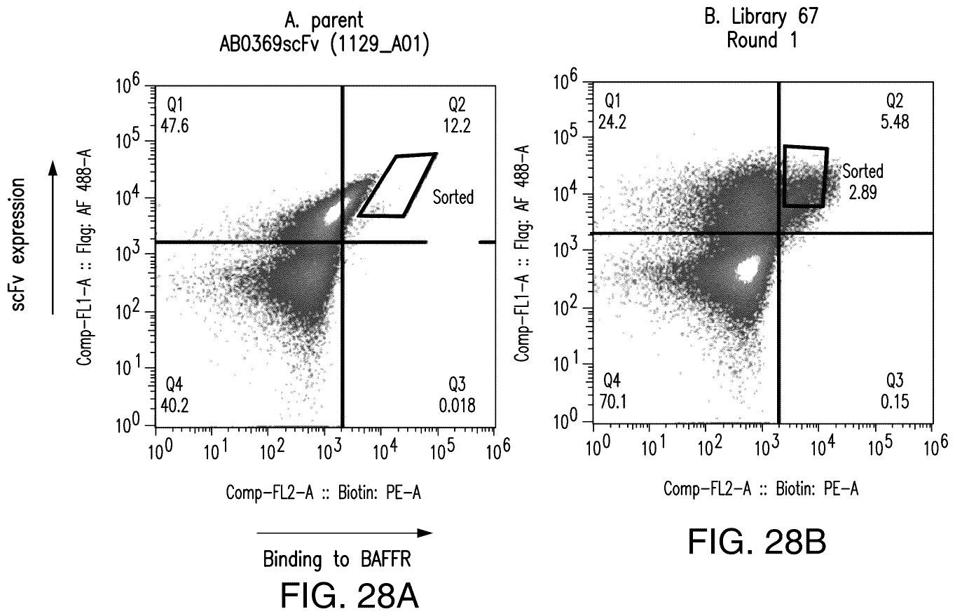

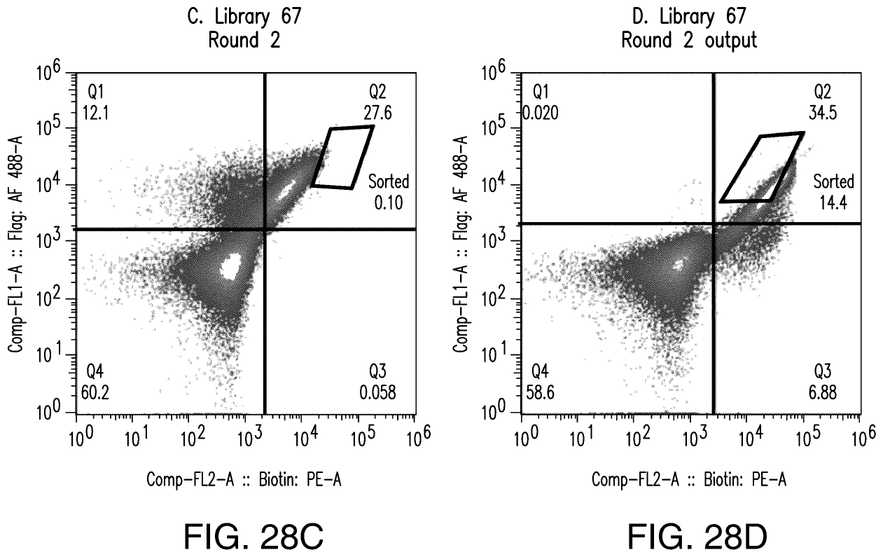

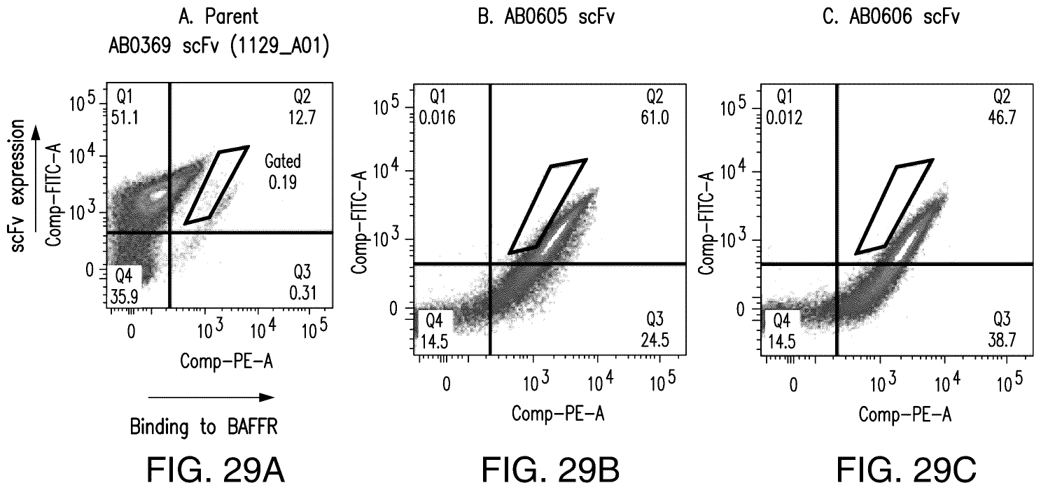

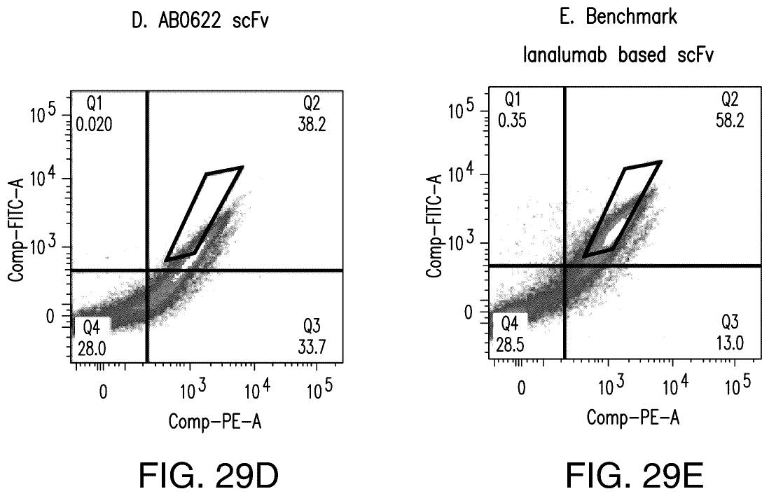

As described above, AB0369 demonstrated specific binding to BAFF-R expressing cells. To search for variants with improved binding affinities, a yeast display affinity maturation library was created by mutating the CDRH3 residue (RFTMLRGLIIEDYGMDV (SEQ ID NO:216)) of AB0369. To enrich for scFvs that have higher affinity towards hBAFF-R, two rounds of selection were carried out with biotinylated hBAFF-R-hFc-His at 1 nM (FIG. 28A-FIG. 28D). The affinities between the parental clone AB0369 and representative individual library clones were compared. Three rounds of FACS sorting resulted in nine clones that contained one or two amino acid differences (bolded) as compared to the parental clone (RFTMLRGWYIEDYGMDV (SEQ ID NO:224); RFTMLRGQYIEDYGMDV (SEQ ID NO:223); RFTMLRGWIIEDYGMDV (SEQ ID NO:225)) and exhibited higher binding affinity for hBAFF-R than the parental clone and parental-derived scFv, using an ianalumab-based scFv as a benchmark control (FIG. 29A-FIG. 29D).

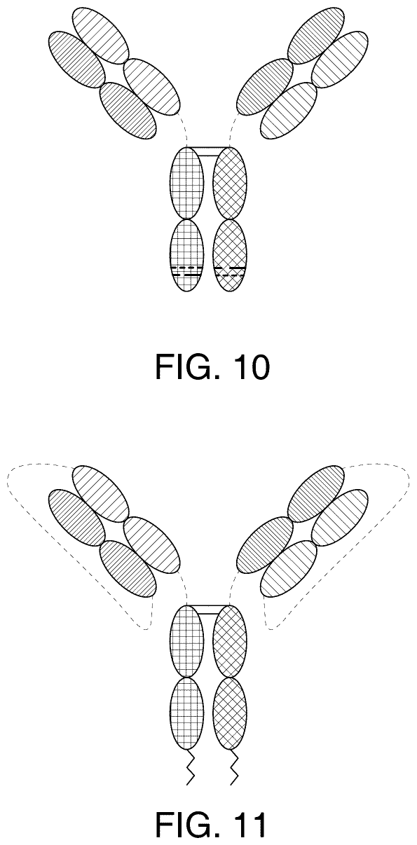

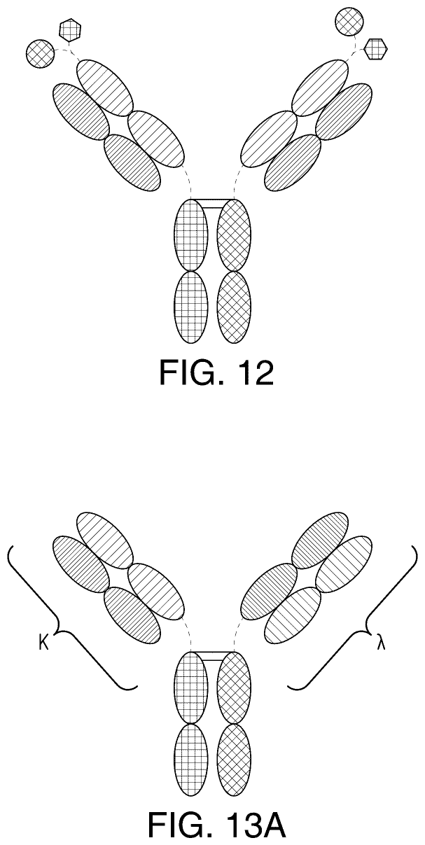

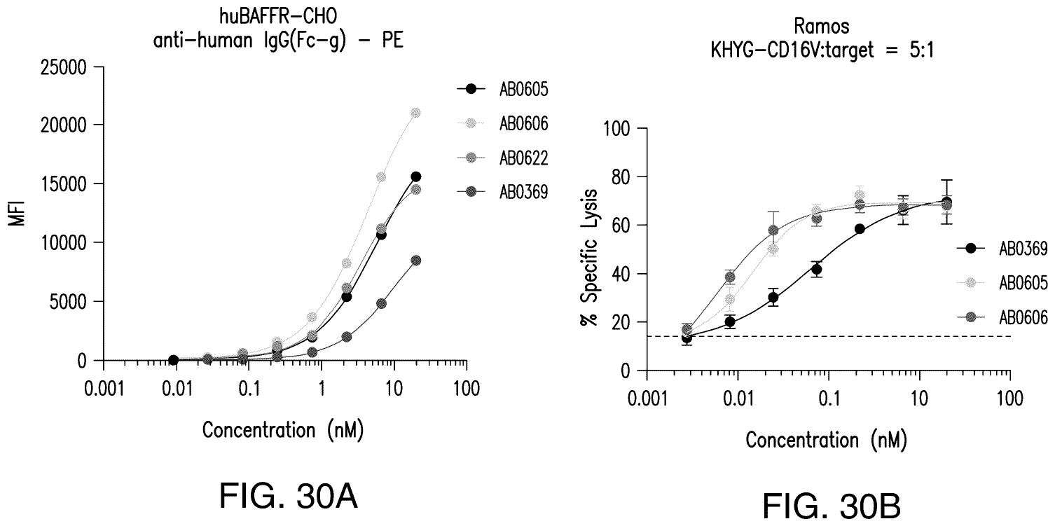

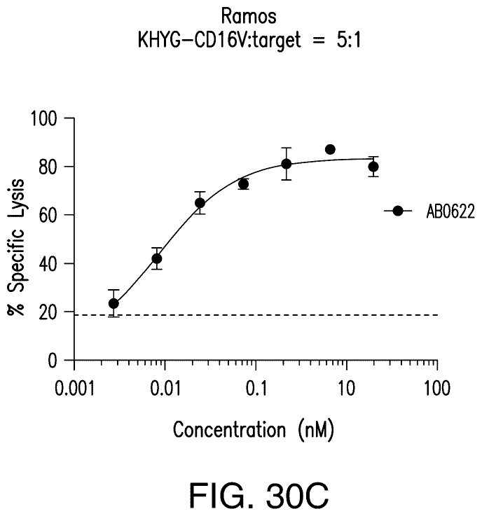

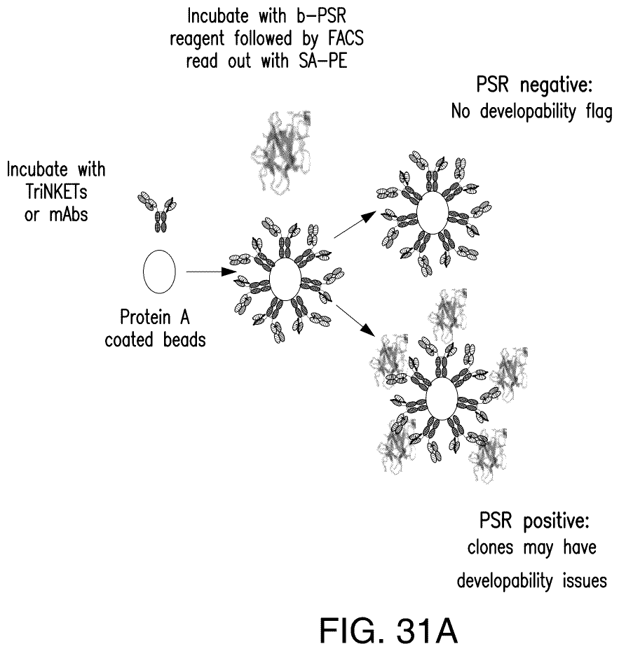

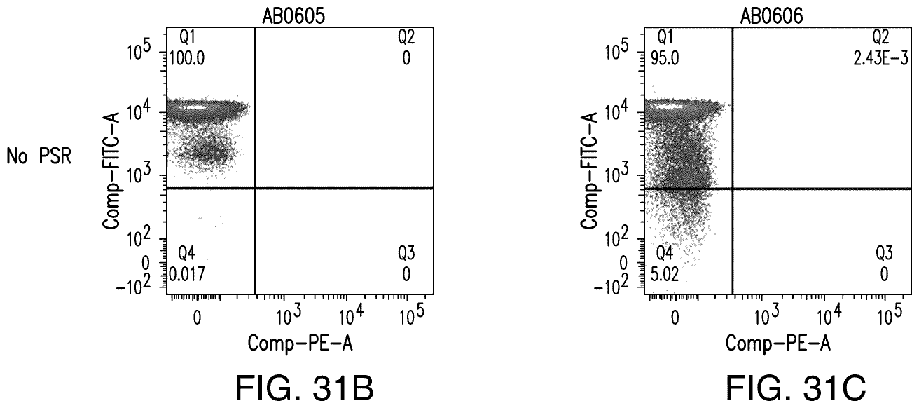

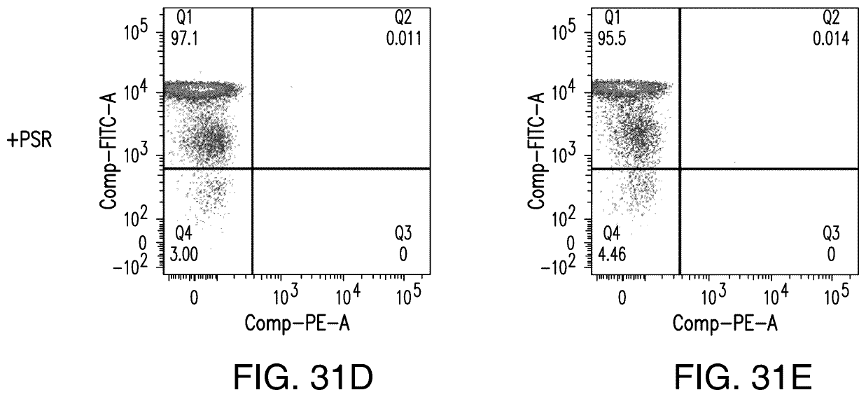

The scFvs with highest hBAFF-R binding affinity were converted into multispecific binding proteins comprising the scFv and two non-BAFF-R binders, expressed in Expi293 cells, and further analyzed for their ability to bind to BAFF-R expressing cells (FIG. 10A) and ability to lyse BAFF-R expressing Ramos cancer cells (FIG. 30B, FIG. 30C). All multispecific binding proteins scored negatively in a poly-specificity assay, suggesting that the improved binding affinity was BAFF-R specific (FIG. 31A-FIG. 31E). Further studies demonstrated greater than three-fold improvement in BAFF-R binding, which translated into six- to ten-fold improvement in potency as measured by EC₅₀ (Table 17). Maximum lysis remained unchanged, suggesting that the improvement in BAFF-R binding affinity was the key driver of this improvement in potency.

TABLE 17
Summary of cell binding and cytolysis demonstrated by
multispecific binding proteins based on HCDR3 affinity
matured variants compared to parental AB0369.

| | | KHYG-A-CD16Av |
|---|---|---|
| | | mediated Cytolysis of |
| | BAFF-R-CHO cell binding | Ramos cells |
| Molecule | EC₅₀ (Nm) | EC₅₀ (Nm) |
| AB0605-001 | 5.72 | 0.15 |
| AB0606-001 | 4.50 | 0.06 |
| AB0622-001 | 3.45 | 0.09 |
| AB0369-001 | >10 | 0.64 |

### CDRH1 and CDRH2 Focused Combinatory Affinity Maturation

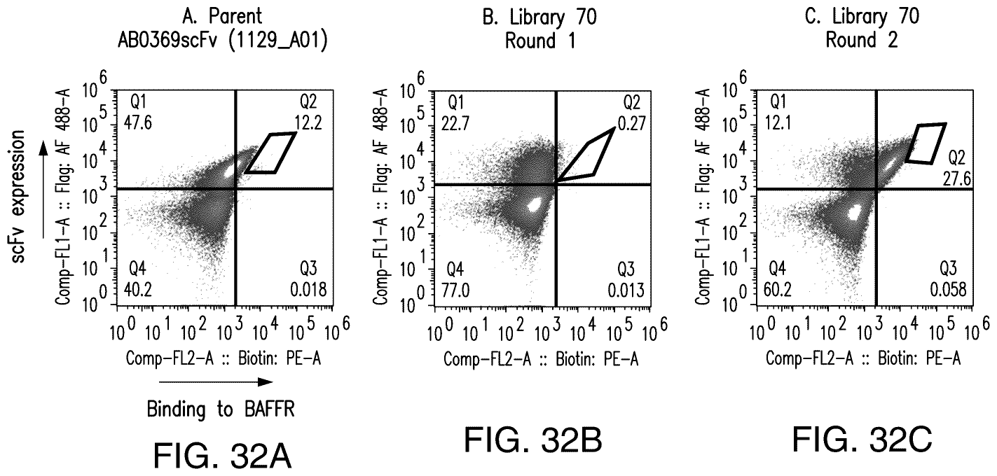

Outcomes from the CDRH3-focused affinity maturation studies demonstrated an improvement in affinity, and further improvement was highly desirable. Thus, the CDRH1 and CDRH2 sequences were selected for affinity maturation (CDRH1: GFTFSSY (SEQ ID NO:214) and CDRH2: WYDGSN (SEQ ID NO:215)) using the matured CDRH3 backbone. The goal was to engineer and select binders with improved affinity over the parental clone (AB0369 scFv) or the CDRH3 optimized variants described above. This created a library with a randomized CDRH1 and CDRH2 while retaining an optimized CDRH3. Two rounds of FACS were performed to enrich for high-affinity binders (FIG. 32A-FIG. 32C).

After FACS, 24 clones were identified. It was observed that several clones with changes in CDRH1 (RFTMLRGWYIEDYGMDV (SEQ ID NO: 224); RFTMLRGQYIEDYGMDV (SEQ ID NO:223)); RFTMLRGWIIEDYGMDV (SEQ ID NO:225)) on the optimized CDRH3 backbone showed a significant improvement in hBAFF-R affinity compared to parental AB0369scFv (1129_A01) (FIG. 33A-FIG. 33D) or to an ianalumab-based scFv benchmark control (the scFv includes a VH and a VL that are based on the VH and VL sequences of ianalumab, but does not contain ADCC-enhancing mutations used in the parent antibody.) (FIG. 33E).

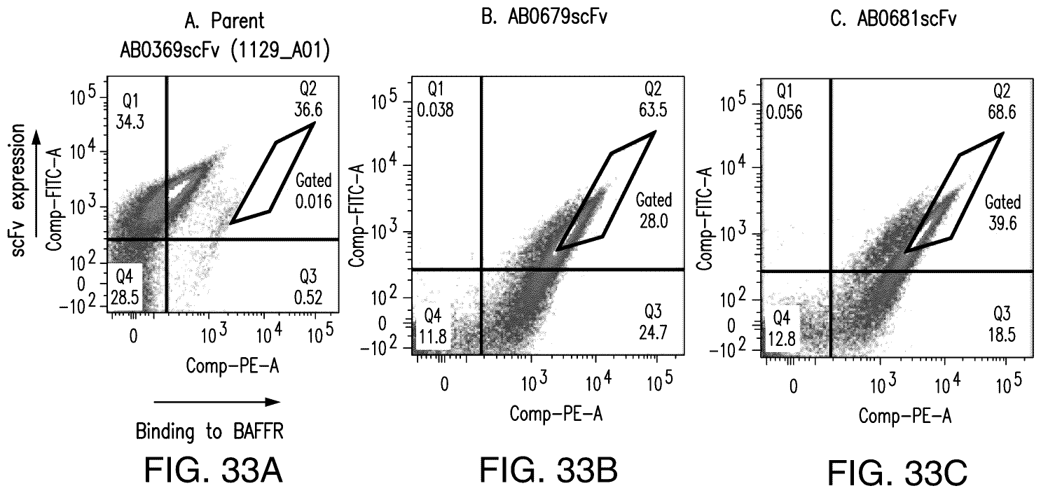

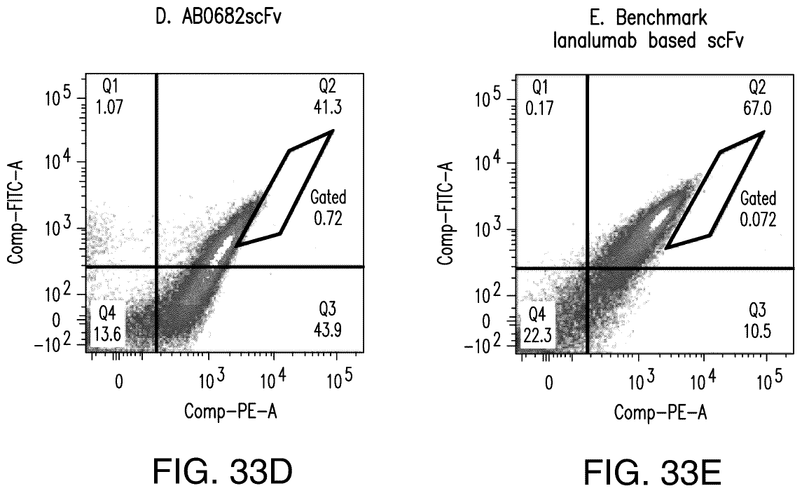

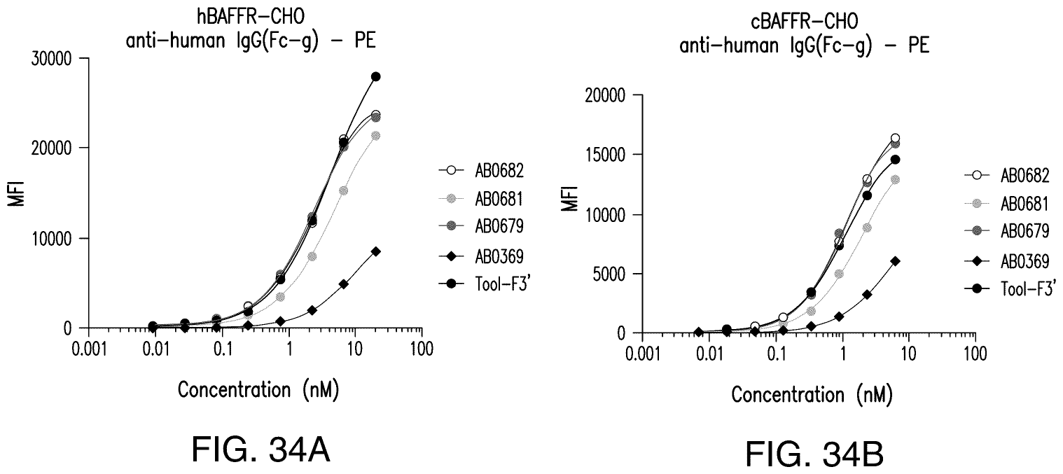

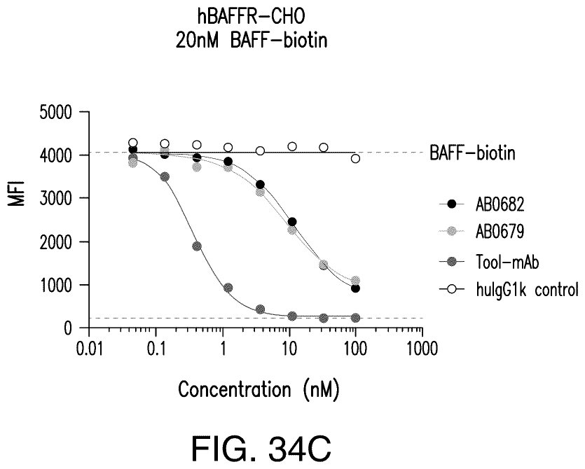

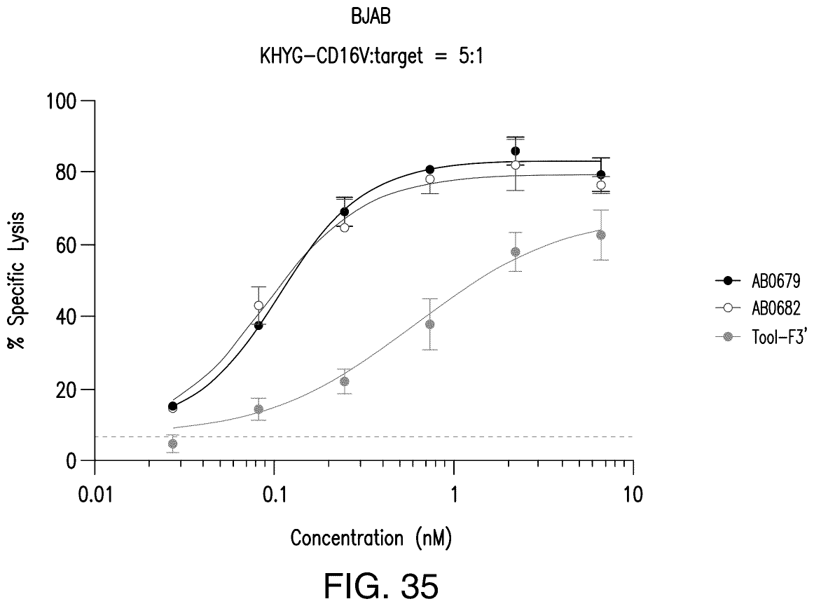

The scFvs with the highest hBAFF-R binding affinities were converted into multispecific binding proteins comprising the scFv and two non-BAFF-R binders, expressed in Expi293 cells, and further analyzed for their ability to bind to human BAFF-R expressing cells (FIG. 34A), to bind to cynomolgus BAFF-R⁺ cells (FIG. 34B), and to inhibit BAFF-R-BAFF interactions (FIG. 34C and Table 18). Tested multispecific binding proteins showed improvement in all three of these criteria and demonstrated efficient killing of BAFF-R⁺ BJAB cells in a KHYG-1-CD16a-mediated cytotoxicity assay (FIG. 35, Table 19).

TABLE 18
Summary of BAFF-R cell binding and BAFF-R-BAFF blocking
demonstrated by multispecific binding proteins based on
CDRH1 and CDRH2 affinity maturation

| | Human BAFF- | Cyno BAFF- | BAFF-BAFF-R |
|---|---|---|---|
| | R cell binding | R cell binding | blocking |
| Molecule | EC₅₀ (nM) | EC₅₀ (nM) | IC₅₀ (nM) |
| AB0682-001 | 2.4 | 3.1 | 12.6 |
| AB0681-001 | 5.1 | 5.6 | — |
| AB0679-001 | 2.3 | 2.6 | 9.4 |
| AB0369-001 | >10 | >15 | — |
| Tool-F3′ | 4.3 | 2.9 | — |
| Tool-mAb | — | — | 0.38 |

TABLE 19
Potency of representative multispecific binding proteins based on CDRH1
and CDRH2 affinity maturation in a KHYG-1-CD16V cytolysis assay.

| Molecule | EC₅₀ (nM) | Max Lysis (%) |
|---|---|---|
| AB0679-001 | 0.11 | 83 |
| AB0682-001 | 0.09 | 79 |
| Tool-F3′ | 0.61 | 69 |

### Remediation of Potential Sequence Liabilities

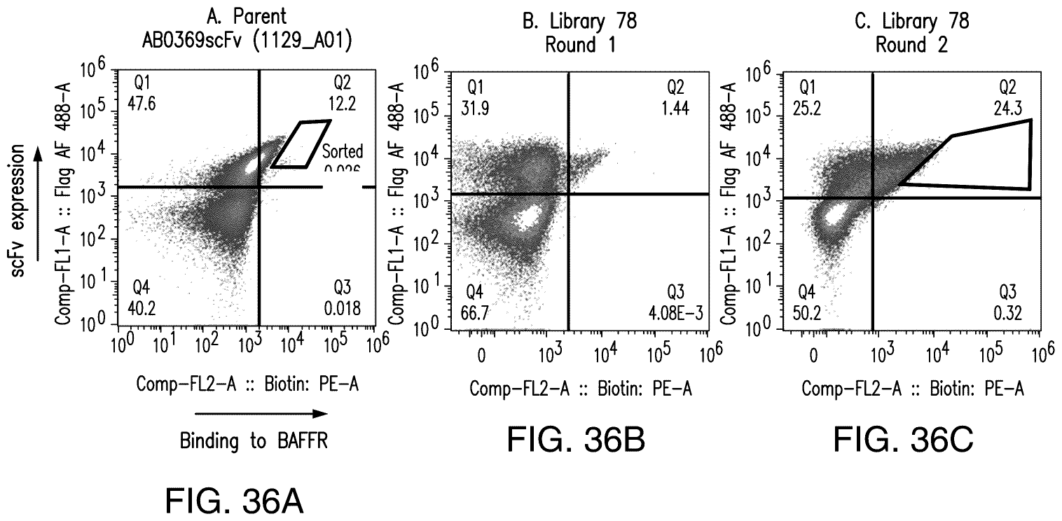

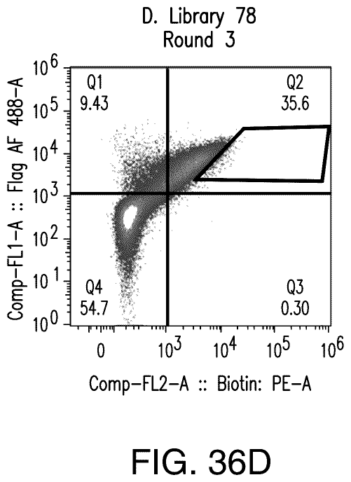

Because the affinity-matured clones contained amino acids in their CDRs that could negatively impact protein expression, stability, or immunogenicity, additional libraries were constructed to select for clones without these amino acids. Three rounds of selection were performed with 1 nM biotinylated hBAFF-R-hFc-His protein leading to enrichment of high affinity binders (FIG. 36A-FIG. 36D). 23 binders were identified altogether, 12 of which were predicted to be free of undesirable amino acids (“liability-corrected”).

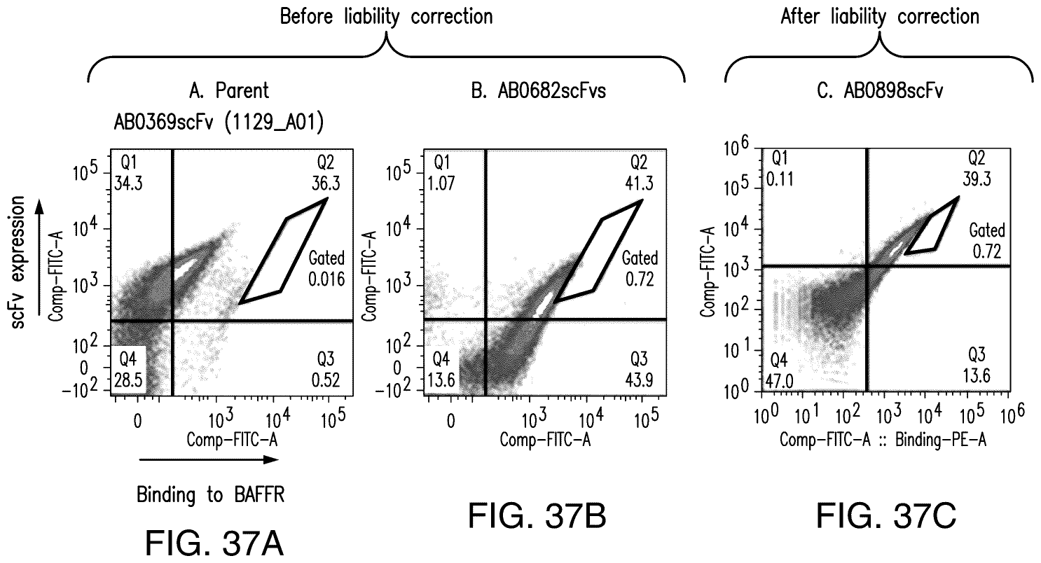

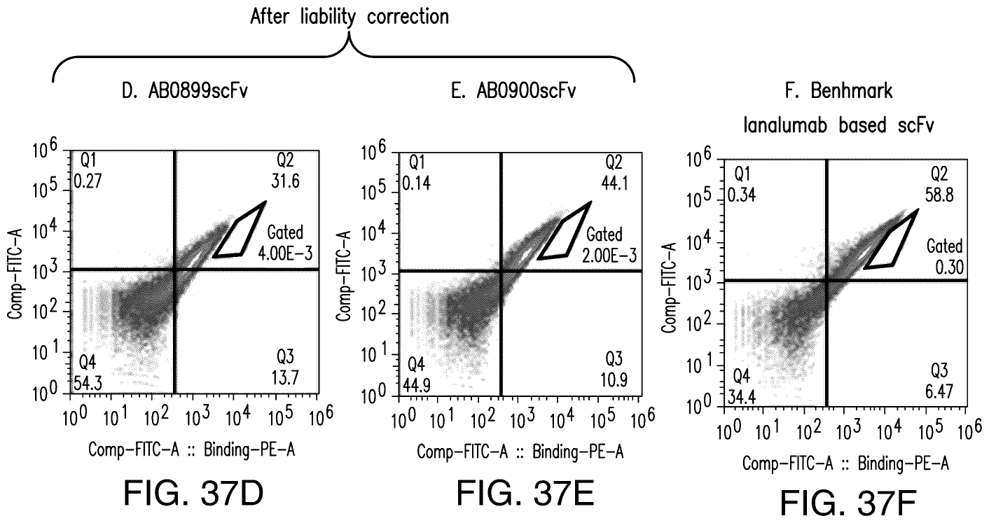

Preferred clones from these libraries included AB0898, (the liability-corrected version of AB0682 described above), AB0899, and AB0900, which were successfully identified and tested for their binding to hBAFF-R while displayed on yeast. All clones showed higher affinity towards hBAFF-R than the parent, AB0369scFv (FIG. 37A-FIG. 37F).

### Characterization of Liability-Corrected Multispecific Binding Proteins

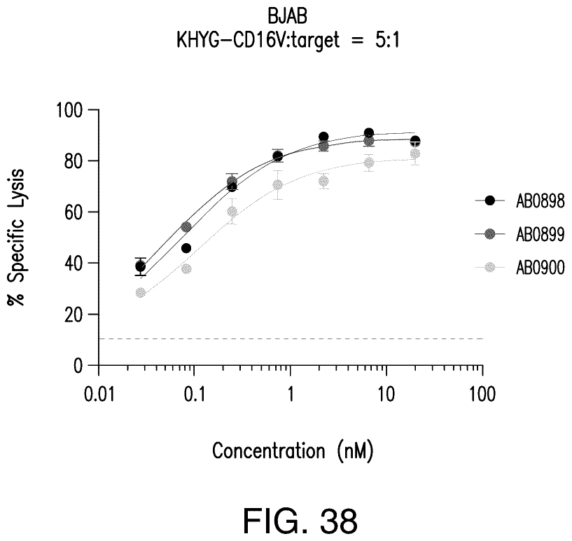

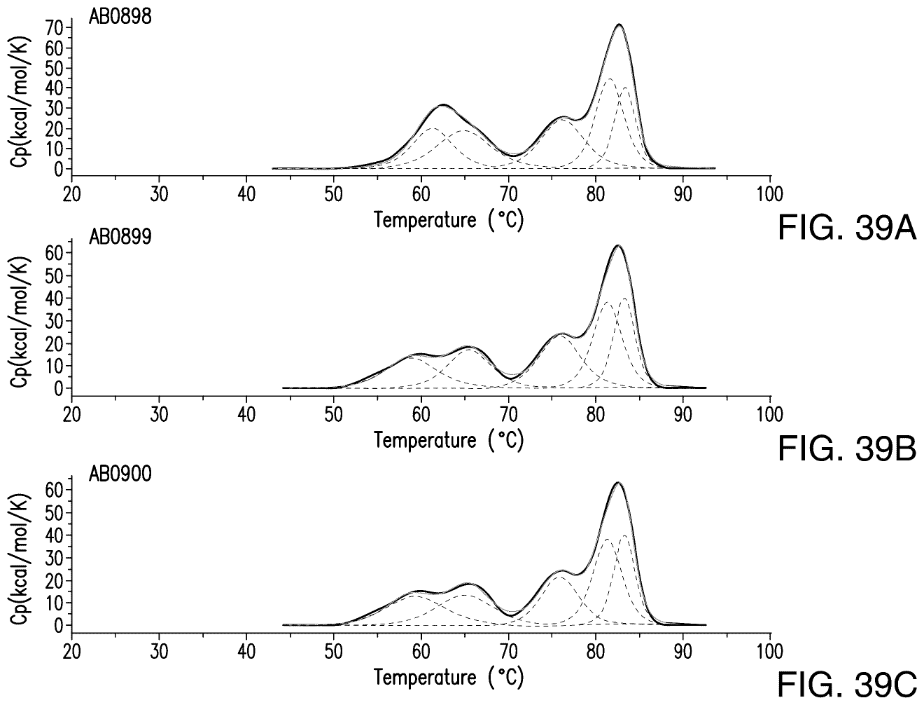

Three of the liability-corrected clones were converted into multispecific binding proteins comprising the scFv and two non-BAFF-R binders, expressed in Expi293 cells, purified by a two-step purification process and characterized by size-exclusion chromatography (SEC), differential scanning calorimetry (DSC), binding to BAFF-R-expressing cells, and ability to lyse BJAB cells in a KHYG-1-CD16aV-mediated cytotoxicity assay. Characterization of these clones is summarized in Table 20 and demonstrates that the liability correction was successful. No negative effect on cell binding was observed and all three clones demonstrated potent killing of BAFF-R-expressing tumor cells (FIG. 38). However, the thermostability of the molecules was Tₘ₁>65° C., as shown in FIG. 39A-FIG. 39C.

TABLE 20
Summary of characterization of multispecific binding proteins
expressing sequence liability corrected BAFF-R binders.

| | SEC, | DSC, | hBAFF-R⁺ | KHYG-A-CD16aV mediated |
|---|---|---|---|---|
| Test | monomer | Tₘ₁ | cell binding | Cytolysis of BJAB cells, |
| Article | (%) | (° C.) | EC₅₀ (nM) | EC₅₀ (nM) |
| AB0898 | 86.9 | 61.23 | 4.3 | 0.08 |
| AB0899 | 96.5 | 58.79 | 4.3 | 0.06 |
| AB0900 | 90.0 | 59.41 | 11.7 | 0.11 |

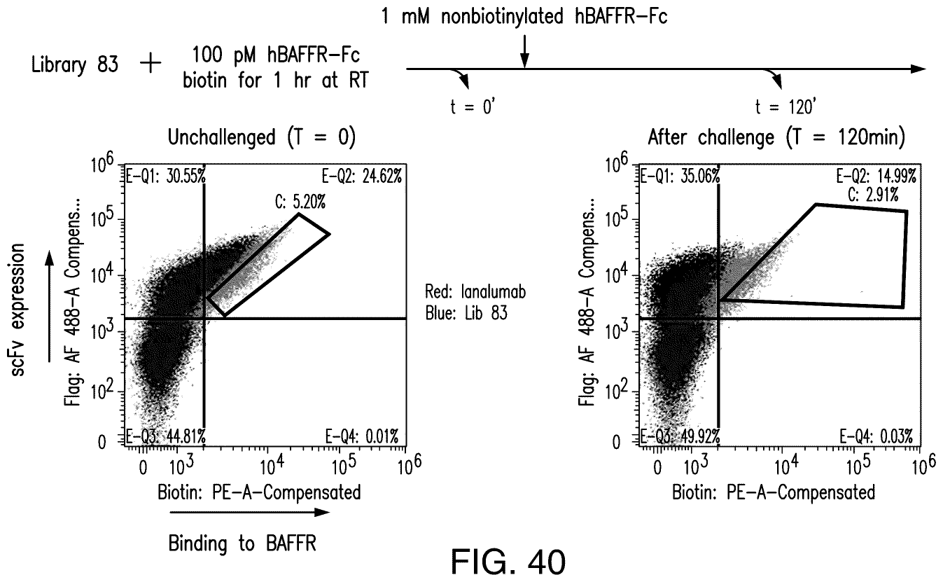

As described above, replacement of the potential sequence liability residues with certain amino acids in CDRs had minimal effect on binding affinity; however, BAFF-R expressing cell binding and thermostability data suggested that further improvement was desirable. Thus, the CDRH1 and CDRH2 sequences (CDRH1: GFTFSSY (SEQ ID NO:214) and CDRH2: WYDGSN (SEQ ID NO:215)) were affinity matured into the liability-corrected CDRH3 backbone, and off-rate pressure was applied to select high affinity clones. Briefly, clones were preincubated with biotinylated hBAFF-R-hFc-His at 100 μM concentration, and then challenged with 1 μM non-biotinylated hBAFF-R-hFc-His for 2 hours. Yeast displaying anti-BAFF-R scFvs that remained bound to biotinylated hBAFF-R-hFc-His were sorted and the process was repeated three times to enrich for high affinity binders with slower off-rate. As shown in FIG. 40, clones remained bound to biotinylated hBAFF-R-hFc-His even after the off-rate pressure challenge, whereas the ianalumab-based scFv benchmark control lost binding to biotinylated hBAFF-R-hFc-His under these conditions, suggesting a slower dissociation rate.

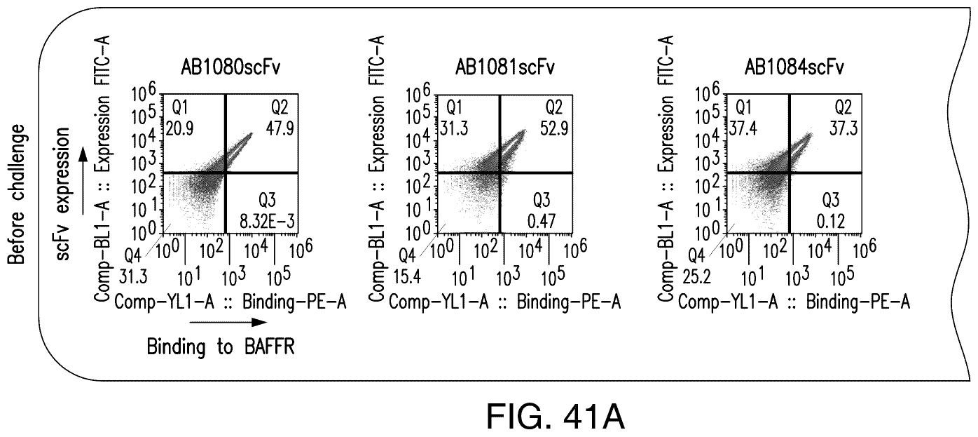

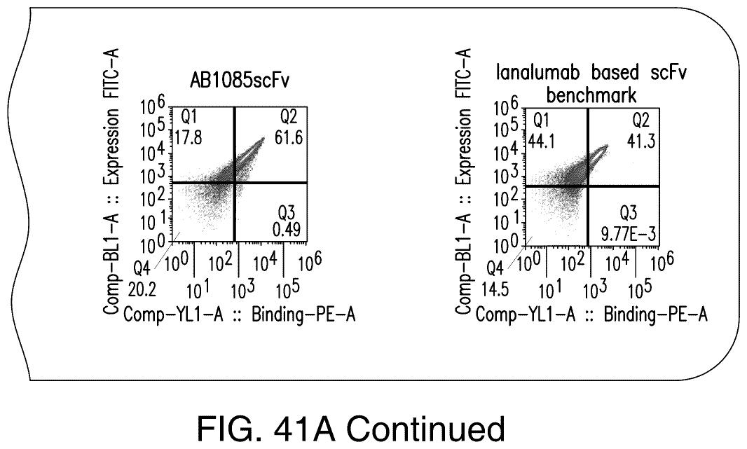

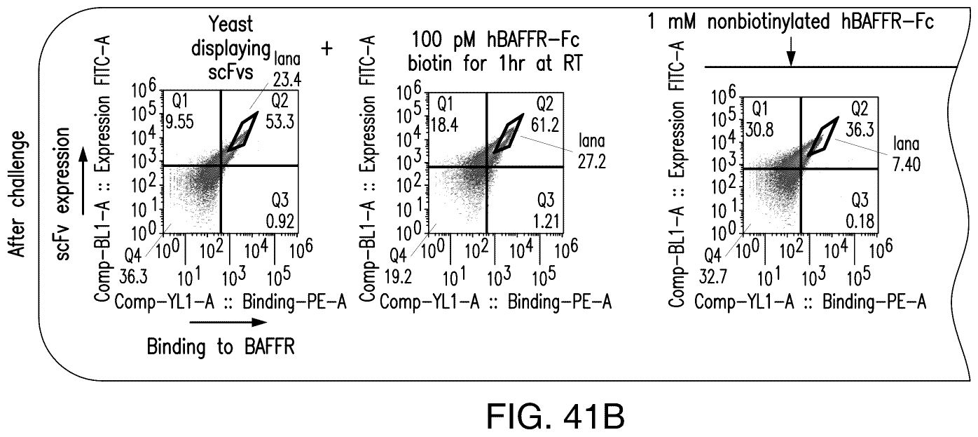

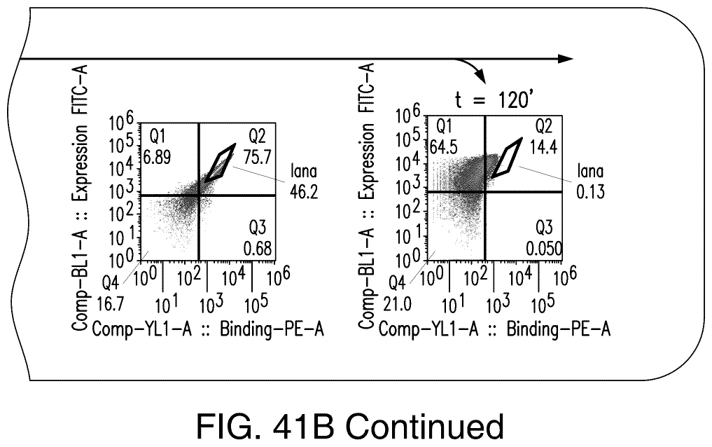

Analysis of individual clones demonstrated high affinity towards hBAFF-R-hFc-His (FIG. 41) and importantly, the clones remained bound to biotinylated hBAFF-R-hFc-His. Notably, ianalumab-based benchmark scFv exhibited loss of binding to biotinylated hBAFF-R-hFc-His after the challenge (FIG. 41A and FIG. 41B). Several of the clones were eliminated from further consideration because they contained additional undesirable amino acids or properties. Sequences of selected clones from the above studies are shown in Table 21.

TABLE 21
CDR sequences of selected clones.

| Sequence | CDRH1 | CDRH2 | CDRH3 |
|---|---|---|---|
| AB1080scFv | GFTFSSY | WYDASN | RFTHLRGWYIEDYGLDV |
| | (SEQ ID NO: 214) | (SEQ ID NO: 233) | (SEQ ID NO: 237) |
| AB1081scFv | GFAFSSY | WYDESN | RFTNLRGWIIEDYGLDV |
| | (SEQ ID NO: 238) | (SEQ ID NO: 239) | (SEQ ID NO: 240) |
| AB1084scFv | GFTFSMY | WYDASN | RFTRLRGWYIEDYGLDV |
| | (SEQ ID NO: 241) | (SEQ ID NO: 233) | (SEQ ID NO: 242) |
| AB1085scFv | GFTFGSY | WYDGSN | RFTHLRGQYIEDYGMDV |
| | (SEQ ID NO: 243) | (SEQ ID NO: 215) | (SEQ ID NO: 244) |

Potential sequence liabilities are bold-underlined and residues demonstrating diversity between clones are bolded.

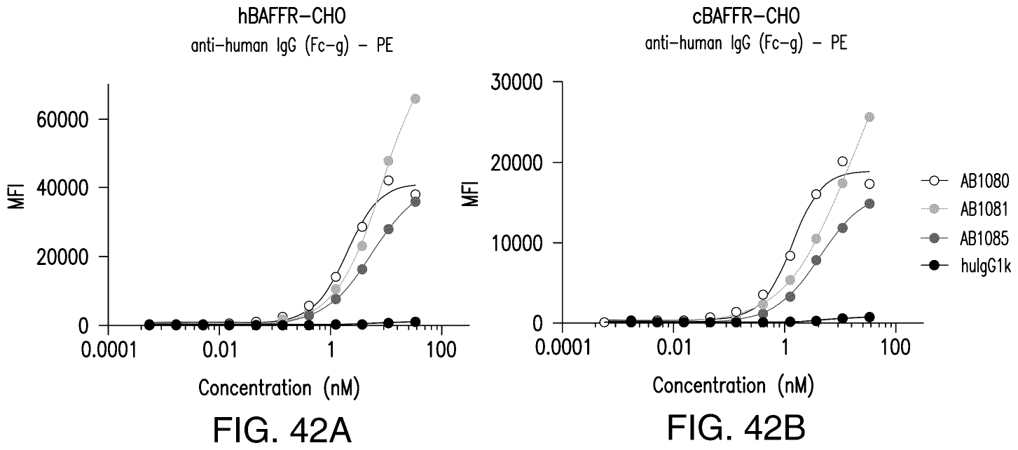

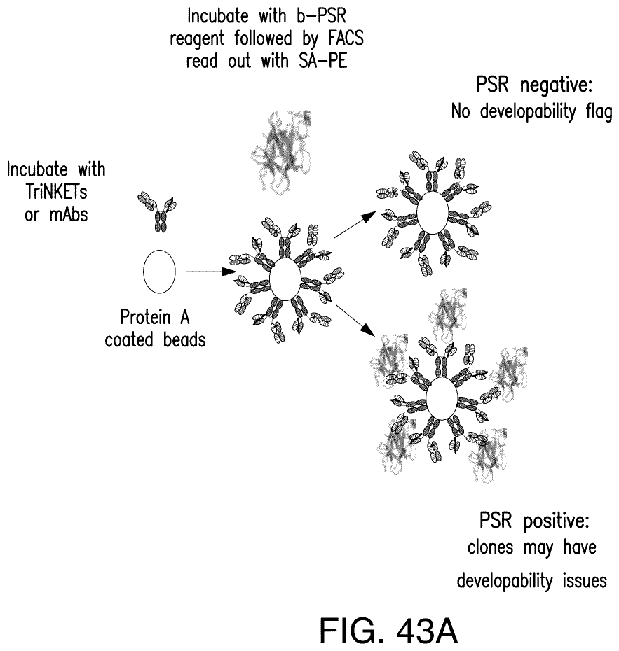

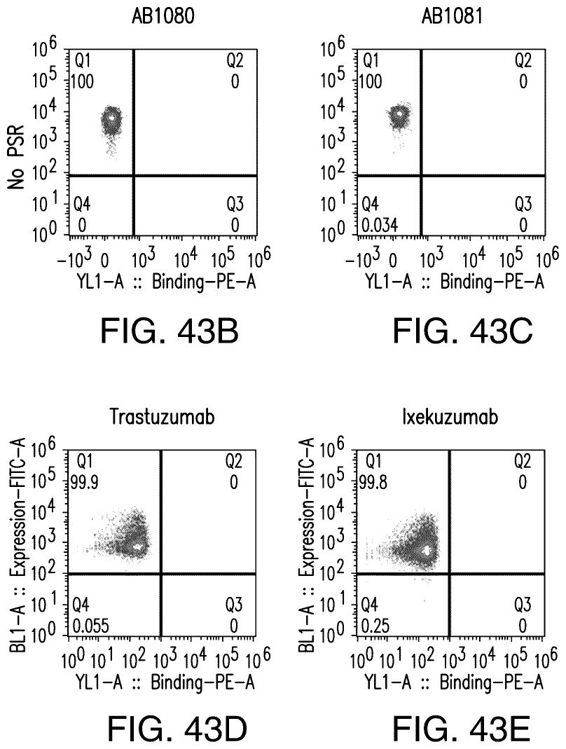

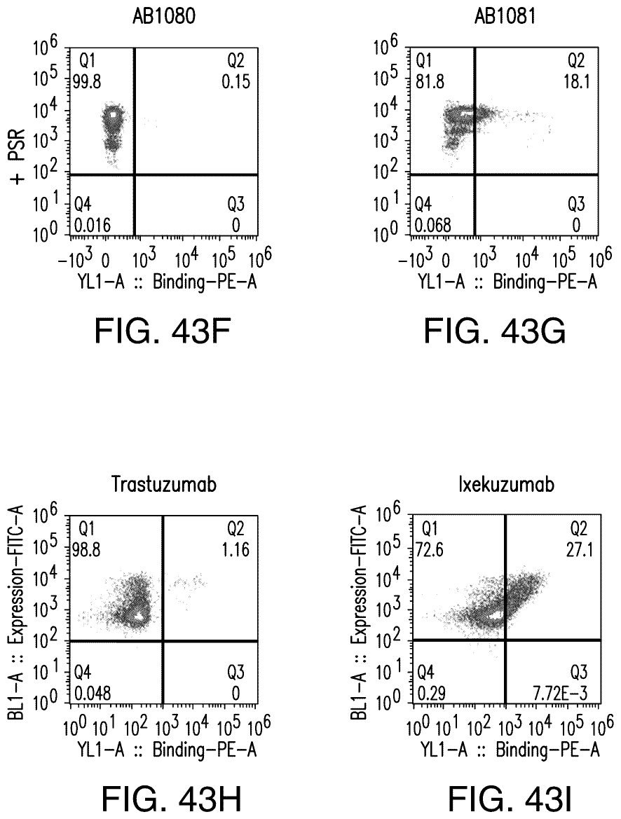

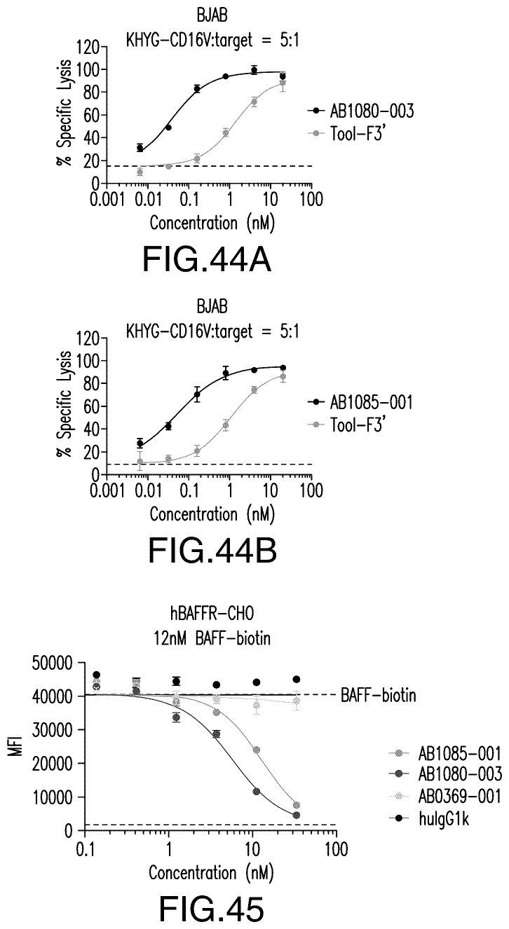

Selected clones from the off-rate challenge studies described above were produced as multispecific binding proteins comprising an scFv of the respective binders and two non-BAFF-R binders, expressed in Expi293 cells, and characterized by binding to hBAFF-R expressing cells and cynomolgus BAFF-R expressing cells, ability to lyse BAFF-R expressing cancer cells in a KHYG-1-CD16aV-mediated cytotoxicity assay, ability to block BAFF-BAFF-R interactions, thermostability (differential scanning fluorimetry, DSF) and hydrophobicity (HIC) (results are summarized in Table 22). Binding affinity of AB1080, AB1081, and AB1085 to BAFF-R⁺ cells was improved as compared to the parental clones (FIG. 42A and FIG. 42B as compared to Table 20). Additionally, binding affinity to cynoBAFF-R was similar to binding affinity to hBAFF-R (FIG. 42A and FIG. 42B). Lack of polyspecificity was confirmed by a PSR assay (FIG. 43A-FIG. 43I). AB1084 was removed from further study due to long retention time on HIC and subsequent potential for higher aggregation propensity. Improved multispecific binding proteins demonstrated vastly higher potency than the multispecific binding protein based on the ianalumab sequence (FIG. 44A and FIG. 44B). In addition, a greater than ten-fold improvement in potency was observed as compared to the original AB0369 multispecific binding protein. Importantly, the ability to block BAFF-BAFF-R binding was significantly improved as compared to the parental AB0369 multispecific binding protein (FIG. 45).

TABLE 22
Summary of characterization of selected multispecific binding proteins.

| | hBAFF-R⁺ | cBAFF_R⁺ | Cytolysis, | HIC, | DSF, |
|---|---|---|---|---|---|
| | Cell binding | Cell binding | EC₅₀ | retention | Tm1 |
| Test Article | EC₅₀ (nM) | EC₅₀ (nM) | (nM) | (min) | (° C.) |
| AB1080-002 | 1.97 | 1.36 | 0.03 | 11.40 | 66.3 |
| AB1081-002 | >8 | >14 | 0.05 | 11.45 | 65.9 |
| AB1084-001 | — | — | 0.06 | 11.79 | 67.5 |
| AB1085-001 | 5.78 | 4.34 | 0.08 | 9.55 | 68.1 |

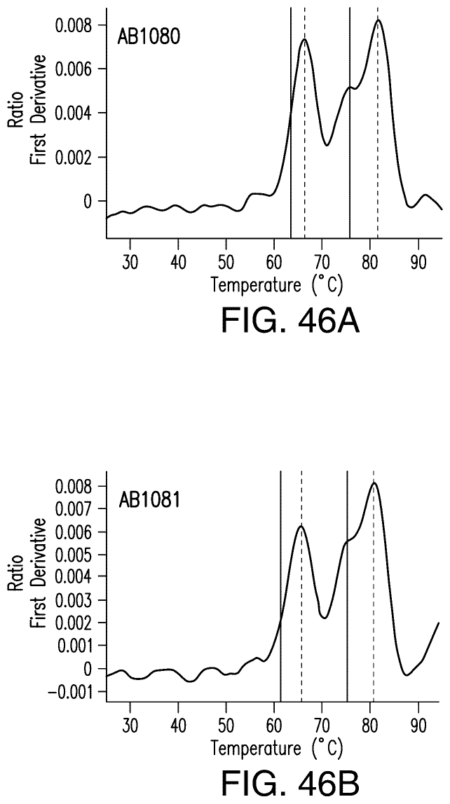

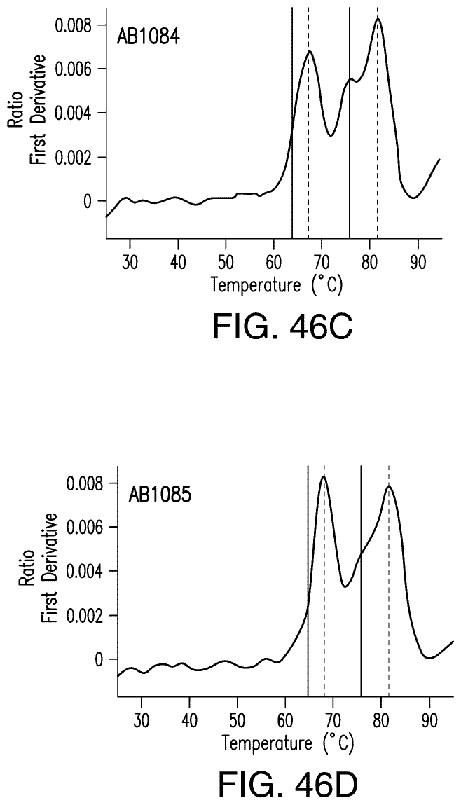

These multispecific binding proteins satisfied criteria for acceptable thermostability as compared to controls adalimumab (Humira) and pembrolizumab (Keytruda) (FIG. 46). HIC chromatograms revealed that AB1080 and AB1081 had retention times of 11.4 and 11.5 min, respectively. AB1085 demonstrated a retention time of 9.5 minutes, which is at the lower edge among approved and late-stage therapeutic antibodies, suggesting very favorable hydrophobic behavior (FIG. 46A-FIG. 46D).

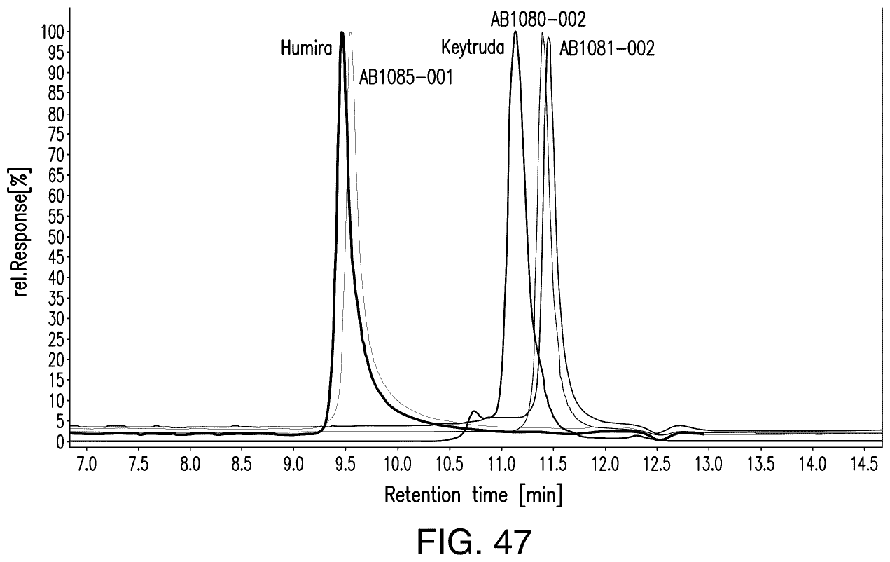

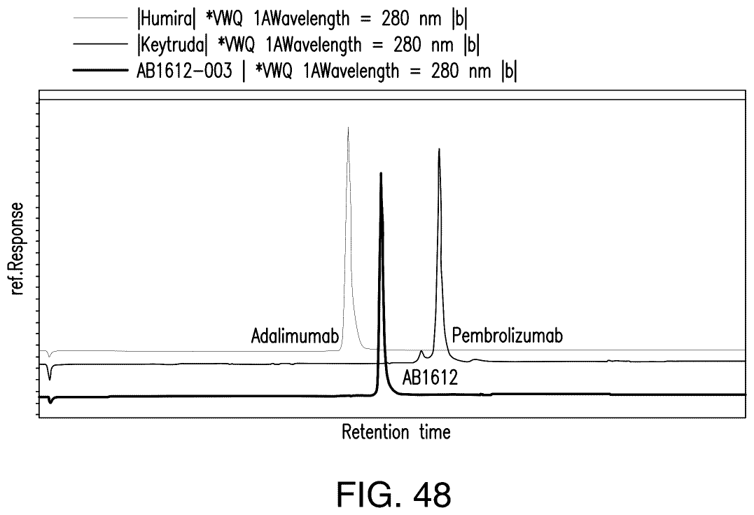

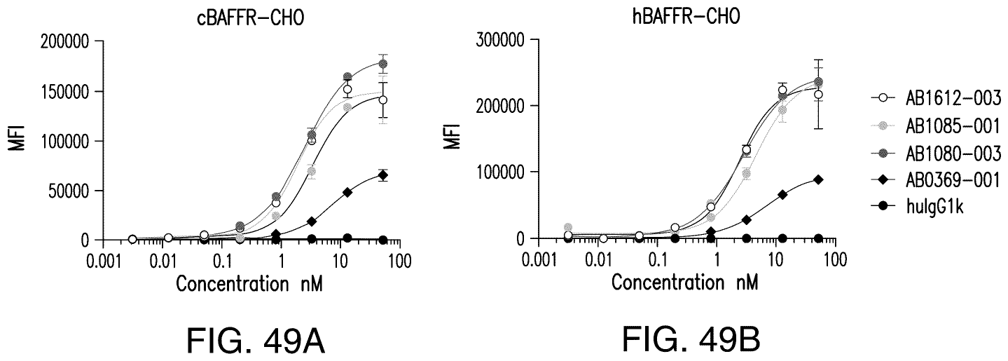

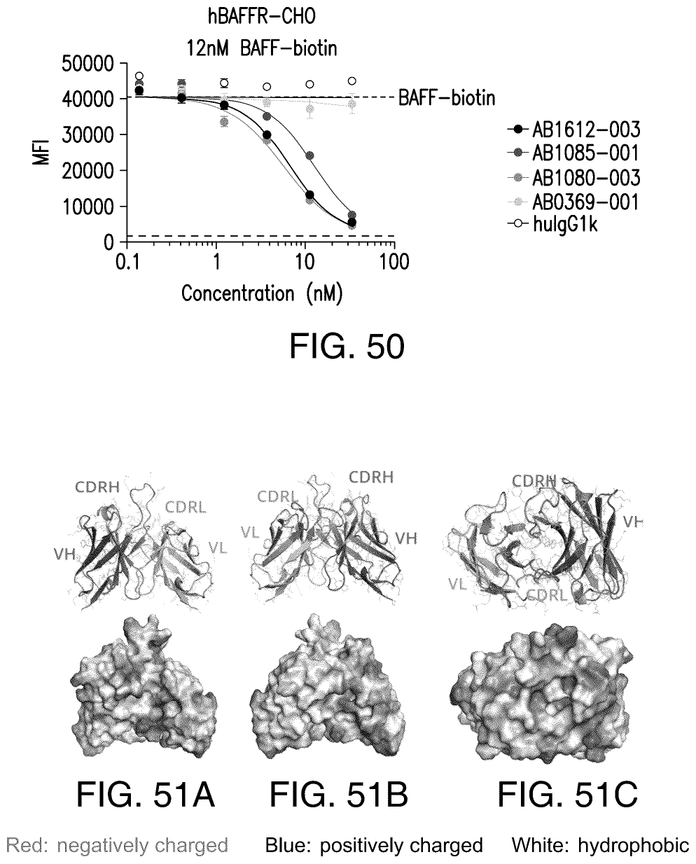

AB1080 and AB1081 showed improved binding to BAFF-R and did not contain any sequence liabilities in the CDR sequences, however, their hydrophobicity was high compared to a panel of benchmarked therapeutic antibodies. AB1085 demonstrated desired hydrophobicity and affinity, but contained potential sequence liabilities in the CDRH2 and CDRH3 sequences (FIG. 47). Sequences of AB1080, AB1081, and AB1085 were compared, and the AB1080 sequence was analyzed and further corrected, with a hydrophobicity reducing mutation W to Q generated (CDRH3: RFTMLRGWYIEDYGMDV (SEQ ID NO:224) to RFTMLRGQYIEDYGMDV (SEQ ID NO:223)). The resulting AB1424/AB1612 multispecific binding protein demonstrated favorable low hydrophobicity that falls within the range of well-behaved biologics (FIG. 48) while maintaining the same high affinity for BAFF-R (Table 23, FIG. 49A and FIG. 49B), potent BAFF-R-BAFF binding blocking (FIG. 50), and comprising a liability-free sequence that is characteristic for parental AB1080 (Table 24).

TABLE 23
Summary of BAFF-R binding and BAFF-R-BAFF blocking by
multispecific binding proteins AB1424/AB1612 lineage.

| | Cyno BAFF-R | Human BAFF-R | |
|---|---|---|---|
| | Cell Binding | Cell Binding | Ligand Blocking |
| Molecule | EC₅₀ (nM) | EC₅₀ (Nm) | IC50 (Nm) |
| AB0369-001 | 7.11 | 6.80 | >1000 |
| AB1080-003 | 2.36 | 2.71 | 5.72 |
| AB1085-001 | 3.29 | 4.48 | 12.67 |
| AB1612-003 | 1.85 | 3.09 | 6.76 |
| Human IgG1k | N/A | N/A | N/A |

TABLE 24
Comparison of BAFF-R binding CDRs in AB1424/AB1612 and its ancestors

| TriNKETs | CDRH1 | CDRH2 | CDRH3 |
|---|---|---|---|
| AB0369 | GFTFSSY | WYDGSN | RFTMLRGLIIEDYGMDV |
| | (SEQ ID NO: 214) | (SEQ ID NO: 215) | (SEQ ID NO: 216) |
| AB1080 | GFTFSSY | WYDASN | RFTHLRGWYIEDYGLDV |
| | (SEQ ID NO: 214) | (SEQ ID NO: 233) | (SEQ ID NO: 237) |
| AB1085 | GFTFGSY | WYDGSN | RFTHLRGQYIEDYGMDV |
| | (SEQ ID NO: 243) | (SEQ ID NO: 215) | (SEQ ID NO: 244) |
| AB1424/AB1612 | GFTFSSY | WYDASN | RFTHLRGQYIEDYGLDV |
| | (SEQ ID NO: 214) | (SEQ ID NO: 233) | (SEQ ID NO: 248) |

In conclusion, two antibody discovery campaigns utilizing recombinant protein and DNA immunization were completed. The first campaign identified four medium affinity non-blocking antibodies. A single binder, AB0369scFv, that was discovered from the second campaign displayed the ability to block BAFF-R-BAFF interactions. Extensive development of AB0396scFv by multiple rounds of affinity maturation, liability correction, and rational sequence design resulted in the binder AB1612/AB1424, which demonstrated desirable properties for a therapeutic candidate.

### Example 4—Molecular Analysis of AB1424/AB1612 F3′ TriNKET Format

In this Example, the molecular format, design, structure, and characteristics of AB1424/AB1612 F3′ TriNKET were analyzed. These studies a) provided basic biochemical and biophysical characterization of the molecule, b) determined the affinity of AB1424/AB1612 F3′ TriNKET for BAFF-R, NKG2D and CD16a (V and F allelic variants), c) confirmed binding of AB1424/AB1612 F3′ TriNKET to cell surface expressed BAFF-R, d) demonstrated selectivity of AB1424/AB1612 F3′ TriNKET, e) and determined the potency of AB1424/AB1612 F3′ TriNKET in killing BAFF-R+ cancer cells.

AB1424/AB1612 F3′ TriNKET is an F3′ format TriNKET as described above, comprising three polypeptides (anti-BAFF-R scFv-CH2-CH3 “Chain S,” SEQ ID NO:270; anti-NKG2D VH-CH1-CH2-CH3, “Chain H,” SEQ ID NO:194; and anti-NKG2D VL-CL, “Chain L,” SEQ ID NO:195). The primary sequence of AB1424/AB1612 F3′ TRINKET was evaluated for the presence of putative sequence liabilities in the CDRs, such as N-linked glycosylation sites, Cys residues, sites of potential deamidation (Asn), oxidation (Met and Trp), isomerization (Asp), and chemically labile bonds (DP). These modifications can impact product efficacy, safety, stability, consistency, or manufacturability.

Analysis of the putative sequence liabilities in the CDRs is provided in Table 25. BAFF-R binding Chain S does not contain any predicted sequence liabilities. NKG2D binding Chain L does not contain any predicted sequence liabilities. NKG2D binding Chain H contains a potential sequence liability that may be prone to truncation in CDRH3. Confirmatory testing demonstrated that AB1424/AB1612 F3′ TriNKET did not show any fragmentation under the conditions of accelerated stability or forced degradation, where the molecule was subjected to thermal, chemical, and mechanical stress, suggesting that the sequence is stable.

TABLE 25
AB1424/AB1612 F3' TriNKET CDR sequences.

| | Variable | | | | |
|---|---|---|---|---|---|
| Chain | region | Framework | CDR1 | CDR2 | CDR3 |
| S | VH | VH3-30 | GFTFSSY | WYDASN | RFTHLRGQYIEDYGLDV |
| | | | (SEQ ID | (SEQ ID | (SEQ ID NO: 248) |
| | | | NO: 214) | NO: 233) | |
| S | VL | VK1-39 | RASQSISSYLN | AASSLQS | QQSYSIPLT |
| | | | (SEQ ID | (SEQ ID | (SEQ ID NO: 249) |
| | | | NO: 217) | NO: 77) | |
| H | VH | VH3-21 | GFTFSSY | SSSSSY | GAPIGAAAGWFDP |
| | | | (SEQ ID | (SEQ ID | (SEQ ID NO: 97) |
| | | | NO: 214) | NO: 290) | |
| L | VL | VK1-12 | RASQGISSWLA | AASSLQS | QQGVSFPRT |
| | | | (SEQ ID | (SEQ ID | (SEQ ID NO: 87) |
| | | | NO: 86) | NO: 77) | |

### Molecular Modeling

Anti-BAFF-R and anti-NKG2D binding arms of AB1424/AB1612 F3′ TriNKET were compared with 377 post-Phase I biotherapeutic molecules using Therapeutic Antibody Profiler (TAP) available at the SabPred website. TAP used AbodyBuilder to generate a model for AB1424/AB1612 F3′ TriNKET with side chains by PEARS. The CDRH3 was built by MODELLER due to its diversity.

Five different parameters were evaluated:
Total CDR length
Patches of surface hydrophobicity (PSH) across the CDR vicinity
Patches of positive charge (PPC) across the CDR vicinity
Patches of negative charge (PNC) across the CDR vicinity
Structural Fv charge symmetry parameter (sFvCSP)

These parameters of AB1424/AB1612 F3′ TriNKET were then compared with the profile distributions of therapeutic antibodies to predict the developability and any potential issues that might cause downstream challenges.

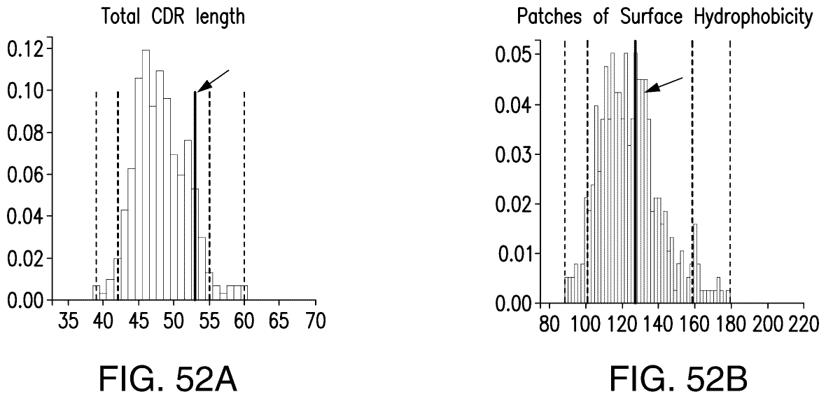

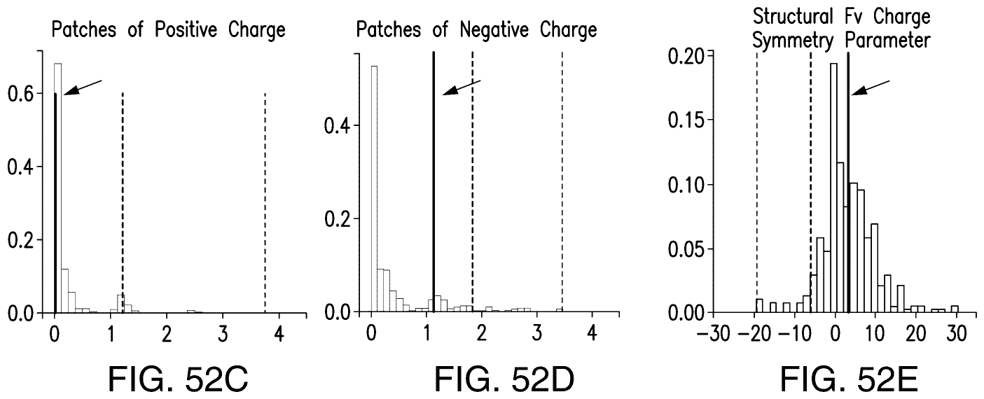

FIG. 51A-FIG. 51C is a model of the variable fragment (Fv) of the BAFF-R binding arm of AB1424/AB1612 F3′ TriNKET in three different orientations (upper panel) and the corresponding surface charge distribution of the same orientation (lower panel). FIG. 52A-FIG. 52E show the total CDR length and surface feature analyses of the BAFF-R binding arm of AB1424/AB1612 F3′ TriNKET. The analysis was performed using the Therapeutic Antibody Profiles (TAP) and was benchmarked with 377 late-stage therapeutic mAbs (Raybould, 2019). The total length of CDRs for the BAFF-R binding arm of AB1424/AB1612 F3′ TriNKET are consistent with those of comparable late-stage therapeutic antibodies (FIG. 52A-FIG. 52E).

The hydrophobicity of a monoclonal antibody is an important biophysical property relevant for its developability into a therapeutic. Hydrophobic patch analysis of the BAFF-R binding arm of AB1424/AB1612 F3′ TriNKET demonstrated that the molecule benchmarks with the vast majority of therapeutic mAbs (FIG. 52A-FIG. 52E). Surface patches of positive and negative charge have been associated with adverse impacts on mAb expression and accelerated in vivo clearance. For the BAFF-R binding arm of AB1424/AB1612 F3′ TriNKET, the positively charged patches, negatively charged patches, and charge symmetry were consistent with the majority of reference mAbs (FIG. 52A-FIG. 52E).

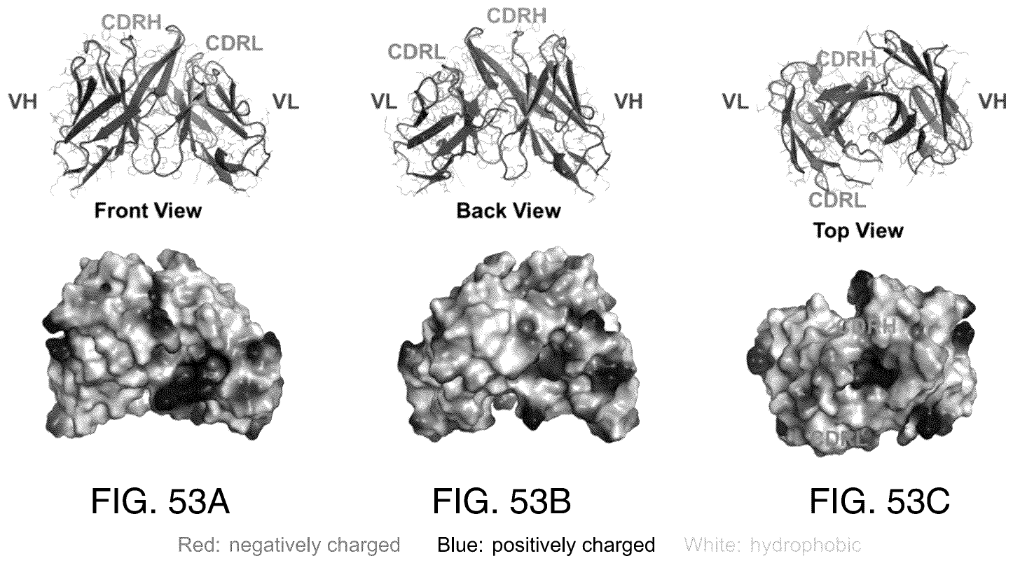

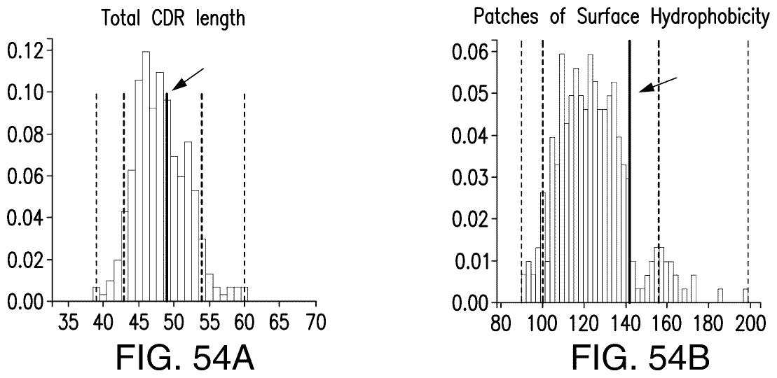

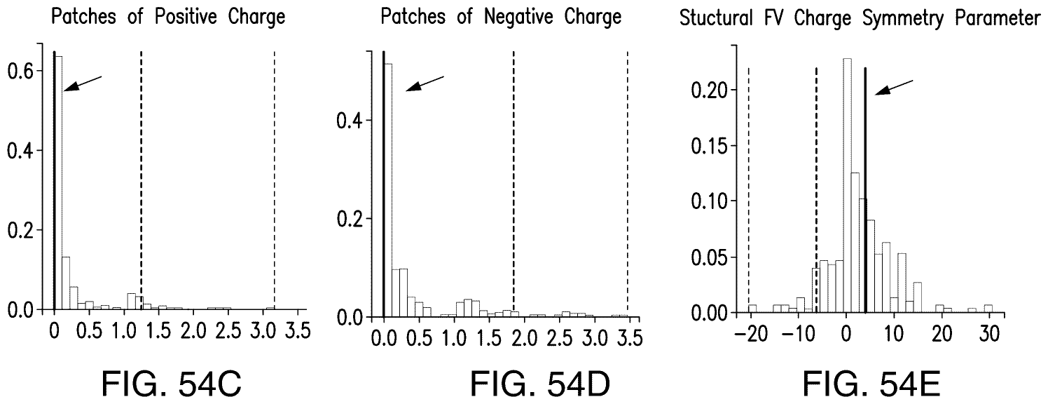

The NKG2D-binding Fab arm was modeled and depicted in three different orientations (FIG. 53A-FIG. 53C, upper panel), and the corresponding surface charge distribution is shown (FIG. 53A-FIG. 53C, lower panel). The surface charge distribution of the NKG2D arm appears to be evenly distributed across the modeled paratope. FIG. 54A-FIG. 54E shows the total CDR length and surface feature analyses of the NKG2D binding arm of AB1424/AB1612 F3′ TriNKET. Analysis was performed using TAP (Raybould, 2019). Total CDR length, hydrophobicity, the positive/negative charge distributions, and the Fv charge symmetry all compared favorably to therapeutic mAb reference data. In summary, there were neither unusual surface charge properties nor unusual patches of surface hydrophobicity identified in these analyses.

### Immunogenicity Assessment

Immunogenicity assessment was performed using the EpiMatrix algorithm from EpiVax. The assessment was performed as described in Cohen et al. (2010) A method for individualizing the prediction of immunogenicity of protein vaccines and biologic therapeutics: individualized T cell epitope measure (iTEM). J. Biomed. Biotechnol. 961752. The T_{reg} adjusted Epimatrix Protein Score, which ranges from −80 (no immunogenicity) to 80 (highly immunogenic), for the sequences of the three chains of AB1424/AB1612 F3′ TriNKET were Chain S: −15.78, Chain L: −23.49, Chain H: −33.39. Thus, the predicted risk of immunogenicity for AB1424/AB1612 F3′ TriNKET appears to be low.

### Hydrophobic Interactions Chromatography

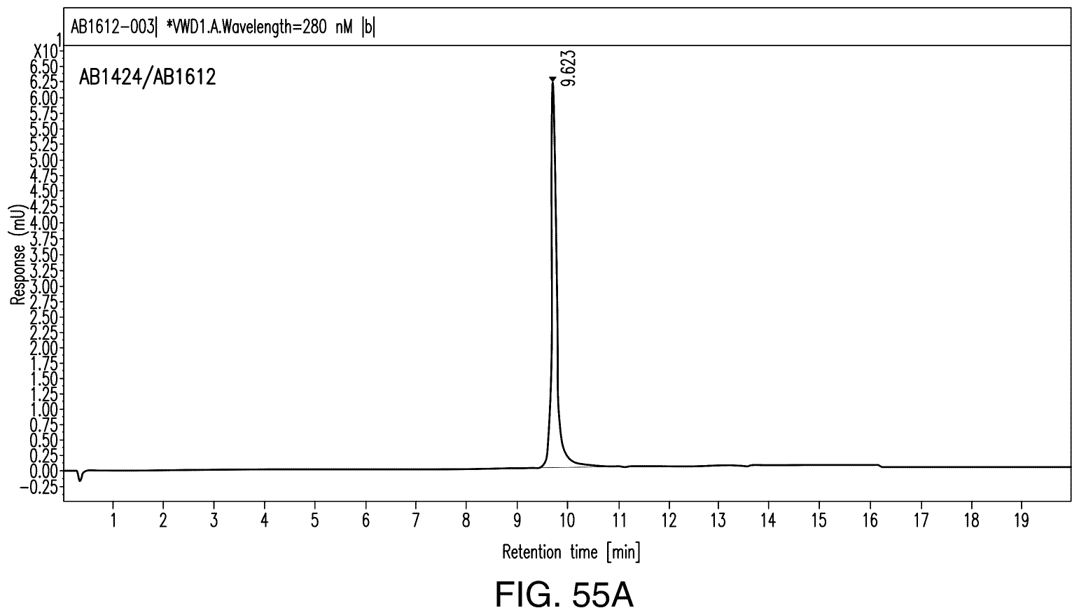

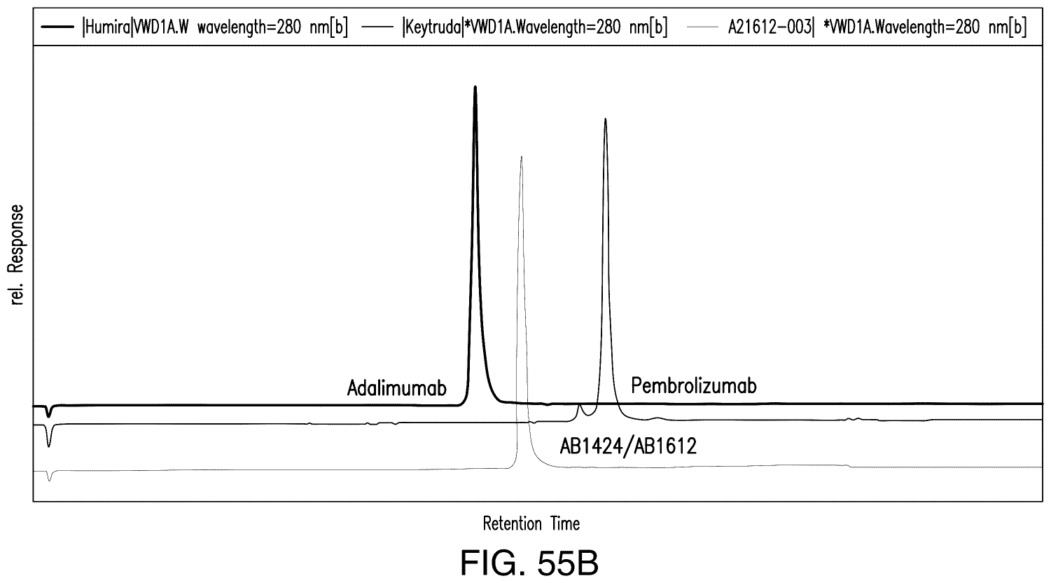

The hydrophobicity prediction data was confirmed by investigating AB1424/AB1612 F3′ TriNKET behavior with analytical Hydrophobic Interactions Chromatography (HIC), a technique that relies upon proteins with significant patches of exposed hydrophobic patches being more prone to aggregation. To perform HIC, briefly, injections of TriNKETs (5 μg of protein) were prepared in a 5:4 ratio of high salt buffer (100 mM sodium phosphate, 1.8 M ammonium sulfate, pH 6.5) to sample. Samples were analyzed using an Agilent 1260 Infinity II HPLC equipped with a Sepax Proteomix HIC Butyl-NP5 5 uM column held at 25° C. The gradient was run from 0% low salt buffer (100 mM sodium phosphate, pH 6.5) to 100% low salt buffer over 6.5 minutes at a flow rate of 1.0 mL/minute. Chromatograms were monitored at 280 nm. Retention times of AB1424/AB1612 F3′ TriNKET on the analytical HIC column is shown in Table 26 and HIC profile in FIG. 55A and FIG. 55B. Commercial adalimumab and pembrolizumab were used as an example of well-behaved biologics and functioned as internal controls for the assay. AB1424/AB1612 F3′ TRINKET had a retention time of 9.7 minutes, compared to 11.3 minutes for pembrolizumab and 8.8 for adalimumab. Thus, experimental hydrophobicity analysis suggested that the hydrophobic properties of AB1424/AB1612 F3′ TriNKET were acceptable for further development.

TABLE 26
Hydrophobicity assessment by HIC.

| Test article | HIC retention time (min) |
|---|---|
| Adalimumab | 8.8 |
| Pembrolizumab | 11.3 |
| AB1424/AB1612 F3′ TriNKET | 9.7 |

Capillary Isoelectric Focusing (cIEF)

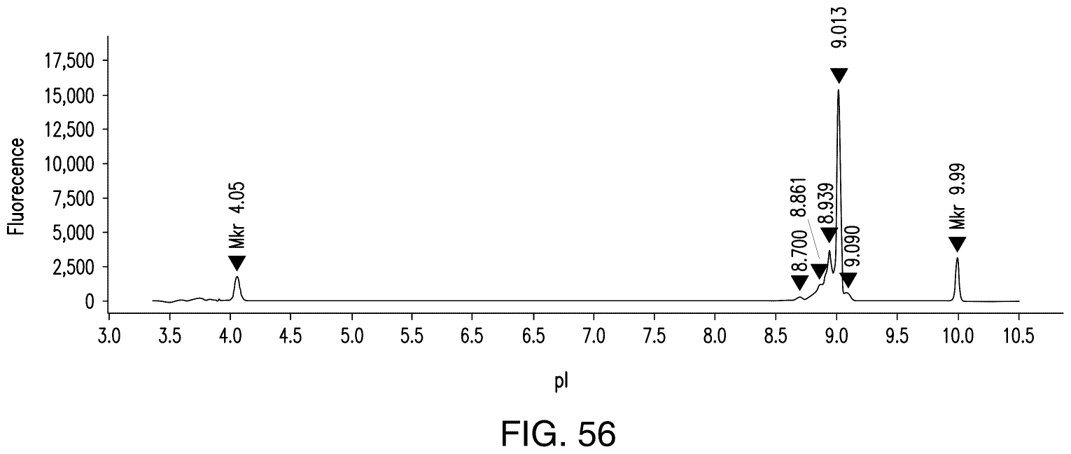

Experimental pI of AB1424/AB1612 F3′ TriNKET was obtained by cIEF (FIG. 56). Briefly, sample was diluted to 1 mg/mL with MilliQ water, 15 μL of sample was added to 60 μL of master mix (water, methyl cellulose, Pharmalyte 3-10, arginine, pI markers 4.05 and 9.99), vortexed, and centrifuged briefly. 60 μL of sample was aspirated from the top of the solution and added to a 96-well plate and centrifuged before testing. The sample was separated for one minute at 1500 volts followed by 8 minutes at 3000 volts on a Maurice instrument (ProteinSimple, San Jose, CA). Commercial grade Trastuzumab was included in the assay as an internal control.

The cIEF profile of AB1424/AB1612 F3′ TriNKET was typical for a monoclonal antibody, with a main peak at pI 9.0 (Table 27). The presence of minor amounts of acidic and basic species was also observed, as shown in Table 28.

TABLE 27
pI determination of AB1424/AB1612 F3′ TriNKET by cIEF.

| Test article | Main peak (pI) |
|---|---|
| Trastuzumab | 8.9 |
| AB1424/AB1612 F3′ TriNKET run 1 | 9.0 |
| AB1424/AB1612 F3′ TriNKET run 2 | 9.0 |
| AB1424/AB1612 F3′ TriNKET Average ± StDev | 9.0 ± 0.0 |

TABLE 28
Summary of AB1424/AB1612 F3′ TriNKET cIEF analysis.

| Test article | pI (Main) | % Acidic | % Main | % Basic |
|---|---|---|---|---|
| AB1424/AB1612 F3′ TriNKET | 9.0 | 41.2 | 53.9 | 4.9 |

### Thermal Stability Analysis

The thermal stability of AB1424/AB1612 F3′ TriNKET was assessed by differential scanning calorimetry (DSC) in PBS pH 7.4 or in HST comprising 20 mM histidine, 250 mM sucrose, 0.01% tween-80 at pH 6.0. To perform DSC, briefly, TriNKETs were diluted to 0.5 mg/mL with PBS. 325 μL were added to a 96-well deep well plate along with a matching buffer blank. Thermograms were generated using a MicroCal PEAQ DSC (Malvern, PA). Temperature was ramped from 20-100° C. at 90° C./hour. Raw thermograms were background subtracted, the baseline model was splined, and data were fitted using a non-two state model.

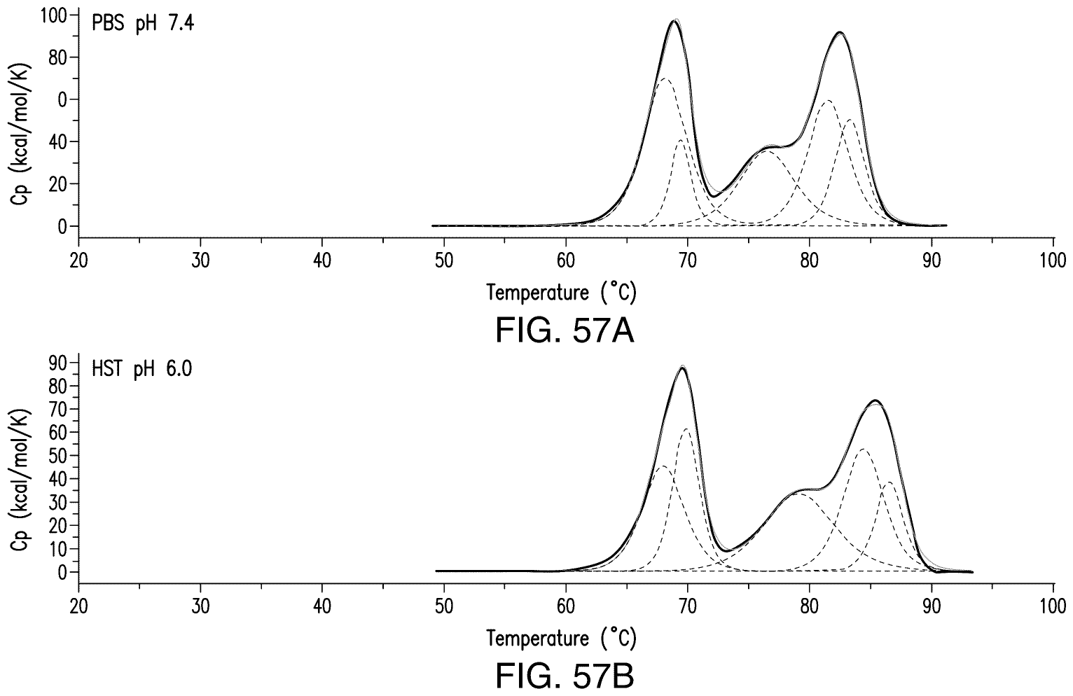

AB1424/AB1612 F3′ TriNKET demonstrated high thermal stability in both buffers (FIG. 57A, FIG. 57B, and Table 29).

TABLE 29
Thermal stability of AB1424/AB1612 F3' TriNKET by DSC.

| Test article | Buffer | Tₒₙₛₑₜ (° C.) | Tₘ₁ (° C.) | Tₘ₂ (° C.) | Tₘ₃ (° C.) | Tₘ₄ (° C.) | Tₘ₅ (° C.) |
|---|---|---|---|---|---|---|---|
| AB1424/AB1612 F3' | PBS | 61.7 | 68.1 | 69.3 | 76.4 | 81.4 | 83.2 |
| TriNKET | | | | | | | |
| AB1424/AB1612 F3' | A | 61.8 | 67.9 | 69.8 | 79.1 | 84.3 | 86.4 |
| TriNKET | | | | | | | |

### Disulfide Bond Arrangement

AB1424/AB16121F3′ TriNKET is an engineered molecule based on the backbone of a monoclonal IgG1 antibody. While a typical IgG1 contains 16 disulfide bonds, the 1F3′ format of AB11424/AB16121F3′ TriNIKET contains only 15 disulfide bonds.

The disulfide bond arrangement of AB11424/AB16121F3′ TriNIKET was confirmed by LC-MS/MS peptide mapping analysis of a non-reduced tryptic digest. Disulfide bonded peptides were identified by MS/MS database searching and confirmed by comparing their intensities in the native and reduced digests. All the standard disulfides expected from the antibody structure were confirmed. A summary of the observed disulfide linked peptides in AB1424/AB16121F3′ TriNIKET is shown in Table 30. All theoretical disulfide linked peptides were observed with high mass accuracy (<2 ppm), were reducible, and were sequence confirmed by MS/MS fragmentation.

TABLE 30
Theoretical and experimental mass of disulfide linked peptides in
AB1424/AB1612 F3′ TriNKET.

| | | | Theoretical | Experimental | Mass accuracy |
|---|---|---|---|---|---|
| Chain | Domain | Peptide | mass (Da) | mass (Da) | (ppm) |
| L | VL | L2:L7 | 4482.0784 | 4482.0842 | 1.7 |
| | CL | L12:L19 | 3555.7490 | 3555.7552 | 1.9 |
| H | VH | H3:H10 | 3371.4686 | 3371.4737 | 1.8 |
| | CH1 | H13:H14-15 | 7916.9194 | 7916.9297 | 1.6 |
| | CH2 | H23:H30 | 2328.0977 | 2328.1024 | 2.0 |
| | CH3 | H37:H41 | 4432.0675 | 4432.0797 | 2.8 |
| S | scFv VH | S3-S11 | 3408.4638 | 3408.4620 | −0.5 |
| | scFv* | S9:S48*^{a} | 1011.4154 | 1011.4155 | 0.0 |
| | scFv VL | S32:S46^{a} | 1316.5853 | 1316.5850 | −0.2 |
| | CH2 | S26:S33 | See H23:H30 | N.A. | N.A. |
| | CH3 | S42:S47 | 3844.8236 | 3844.8321 | 1.8 |
| intermolecular | Hinge | H20-21:S23-24 | 5454.7834 | 5454.7890 | 1.6 |
| | CL-CH1 | L20-21:H19 | 1260.4863 | 1260.4868 | 0.4 |
| | CH3-CH3* | H36:S40-41* | 2757.3830 | 2757.3888 | 2.5 |

*denotes engineered disulfides
^{a}by trypsin and chymotrypsin digestion

### Binding Characteristics of AB1424/AB1612 F3′ TriNKET

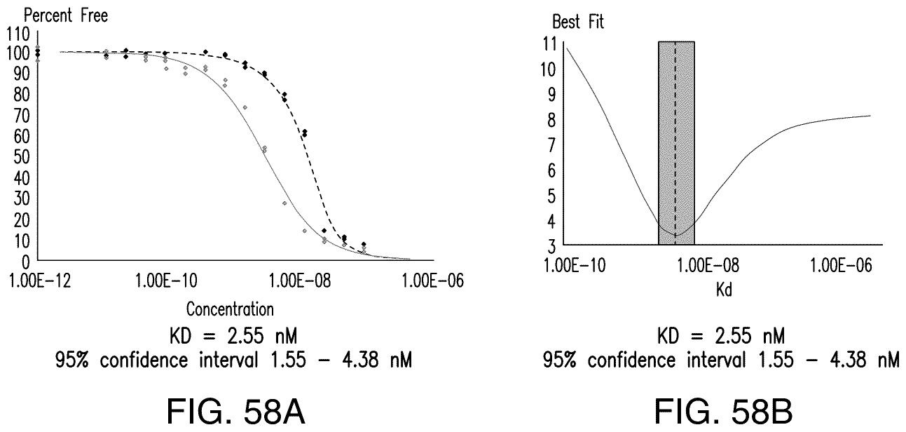

To characterize the affinity of AB1424/AB1612 F3′ TriNKET for human BAFF-R expressed on cells, the kinetic exclusion platform instrument (KinExA) was used. FIG. 58A and FIG. 58B demonstrates that AB1424/AB1612 F3′ TriNKET binds to BAFF-R expressed on the surface of isogenic BAFF-R-CHO cells with a 2.55 nM affinity.

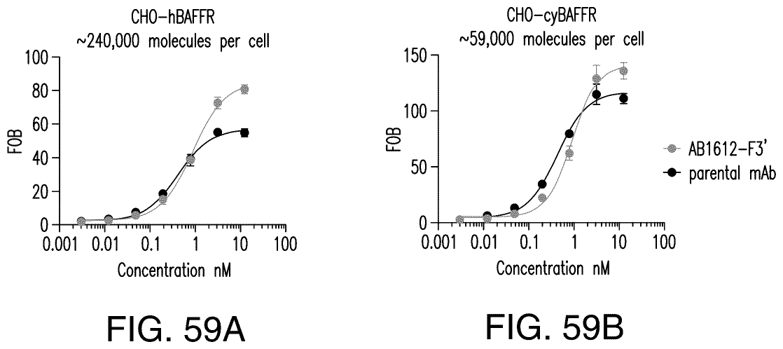

Isogenic cell lines overexpressing human and cynomolgus BAFF-R on the backbone of CHO cell line were designed. AB1424/AB1612 F3′ TriNKET was compared to a corresponding parental antibody (AB1753). AB1424/AB1612 F3′ TriNKET and AB1753 demonstrated a similar dose-response in binding to human and cynomolgus BAFF-R (FIG. 59A and FIG. 59B). The EC₅₀ was nearly identical for AB1424/AB1612 F3′ TriNKET and AB1753 when comparing binding to human and cynomolgus BAFF-R (Table 31). Notably, the fold over background (FOB) was greater for AB1424/AB1612 F3′ TriNKET than AB1753 across both human and cynomolgus BAFF-R cells (FIG. 59A and FIG. 59B and Table 31). Without wishing to be bound by theory, it is hypothesized that this may be attributed to the altered format of AB1424/AB1612 F3′ TRINKET, where antigen engagement is monovalent (rather than bivalent for AB1753) and there is potential for higher loading of the TriNKET on the cell.

TABLE 31
Binding of AB1424/AB1612 F3′ TriNKET and corresponding parental
mAb to isogenic human and cynomolgus BAFF-R-CHO cells.

| | Human | | Cynomolgus | |
|---|---|---|---|---|
| | BAFF-R CHO | | BAFF-R CHO | |
| Test article | EC₅₀ (nM) | Max FOB | EC₅₀ (nM) | Max FOB |
| AB1424/AB1612 F3′ | 0.9 | 85 | 0.9 | 141 |
| TriNKET | | | | |
| Parental mAb | 0.4 | 57 | 0.4 | 116 |
| (AB1753) | | | | |

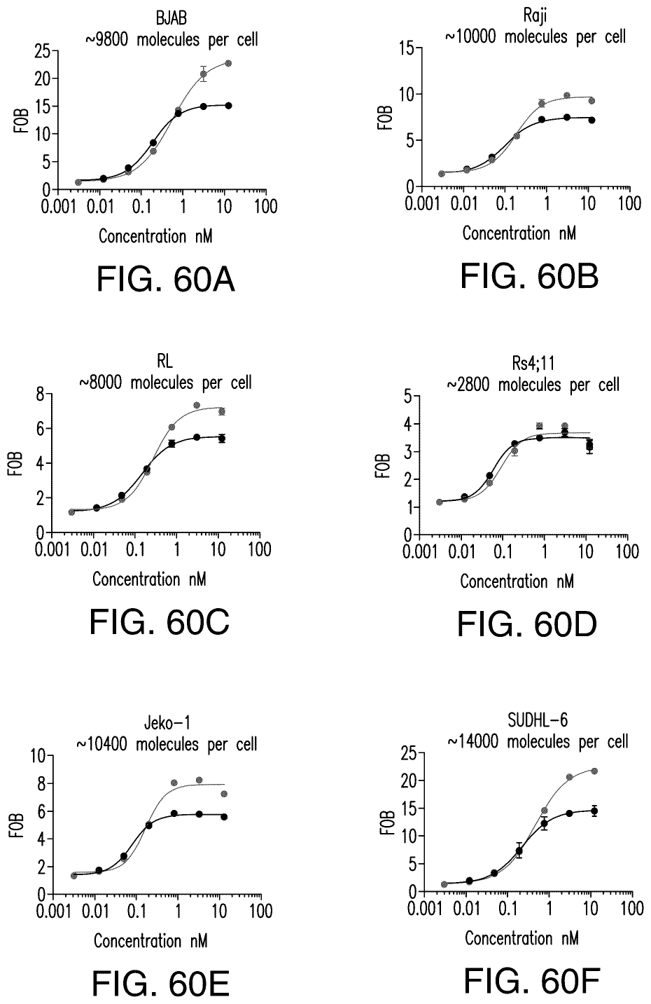

Binding of AB1424/AB1612 F3′ TriNKET to a diverse set of BAFF-R+ cancer cell lines was assessed by flow cytometry. AB1424/AB1612 F3′ TriNKET bound with low nanomolar EC₅₀ to cell-surface BAFF-R on BJAB, Raji, RL, Rs4;11, Jeko-1, and SUDHL-6 cancer cells. The EC₅₀ was comparable amongst the BAFF-R+ cancer cell lines (FIG. 60A-FIG. 60F).

### Binding to NKG2D

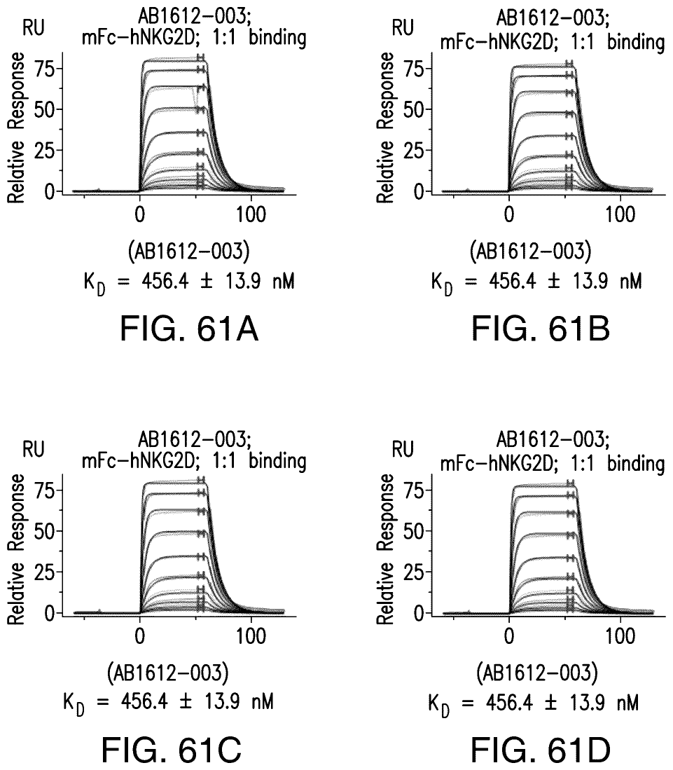

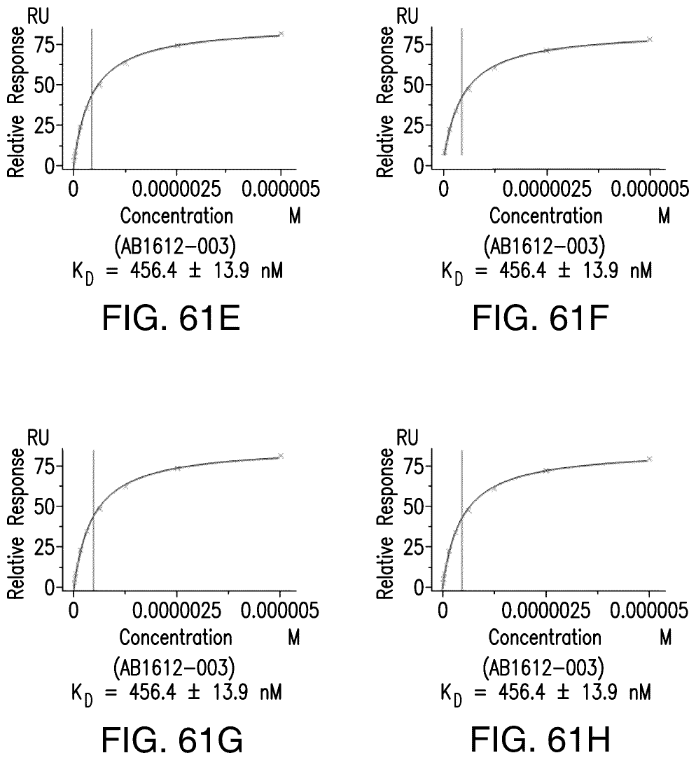

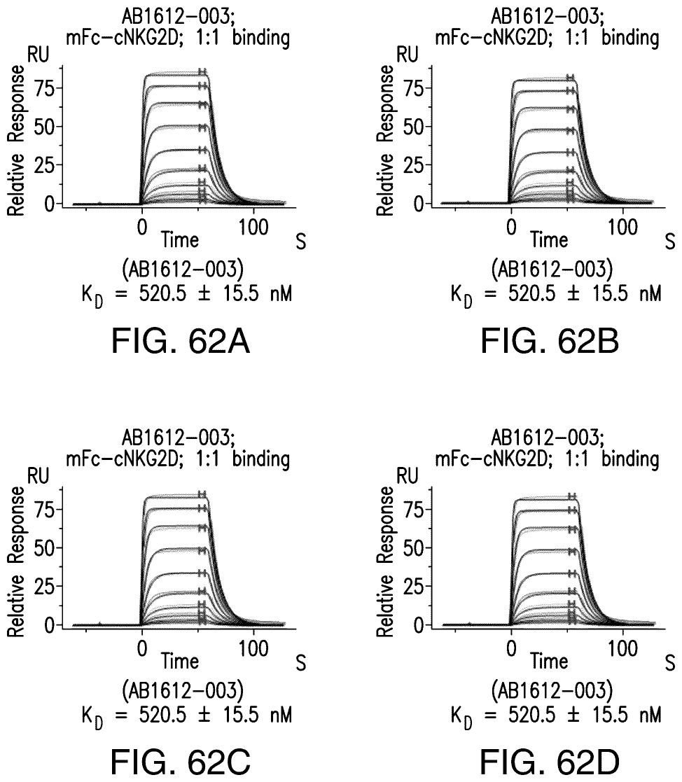

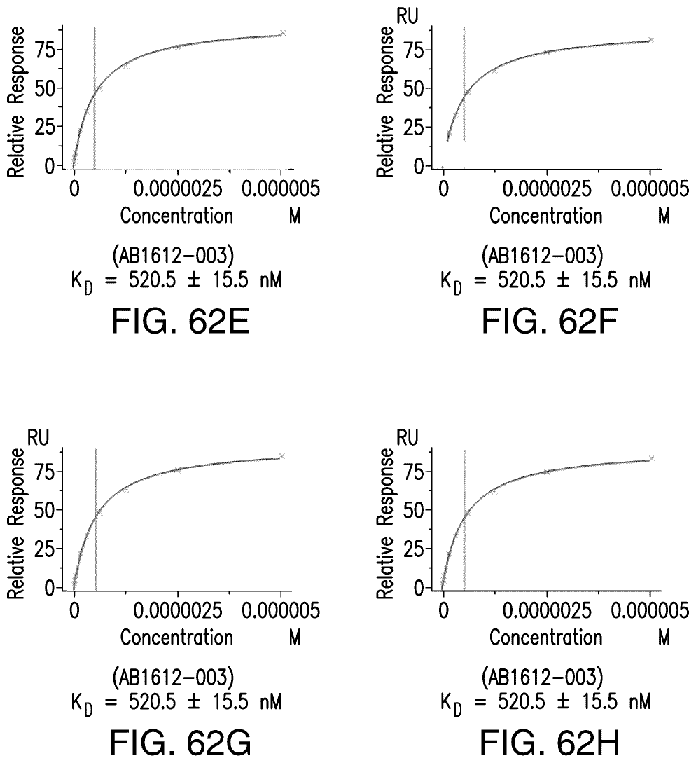

Binding of AB1424/AB1612 F3′ TriNKET to human and cynomolgus NKG2D was assessed by surface plasmon resonance (SPR) (FIG. 61A-FIG. 61H and FIG. 62A-FIG. 62H). NKG2D is a native dimer, therefore recombinant mFc-tagged NKG2D dimer was used for this experiment. Two different fits were utilized to obtain the equilibrium affinity data: steady state affinity fit and kinetic fit. The kinetic constants and equilibrium affinity constants are shown in Table 32 and Table 33. AB1424/AB1612 F3′ TriNKET was designed to bind to human NKG2D with low affinity with a fast rate of dissociation. The dissociation rate constant was 1.1±0.0×10⁻¹ s⁻¹ and 1.1±0.0×10⁻¹ s⁻¹ for human NKG2D and cynomolgus target, respectively. Equilibrium affinity constants (K_{D}) obtained by kinetics fit and steady state affinity fit were very similar for human NKG2D, 455.8±12.7 nM and 456.4±13.9 nM, respectively (Table 32), and cynomolgus NKG2D: 517.0±13.6 nM and 520.5±15.5 nM, respectively (Table 33).

TABLE 32
Kinetic parameters and binding affinities of AB1424/AB1612 F3′ TriNKET for
human NKG2D measured by SPR.

| | | | Kinetics Fit K_{D} | Steady State Fit |
|---|---|---|---|---|
| Test article | kₐ (M⁻¹s⁻¹) × 10⁵ | k_{d} (s⁻¹) × 10⁻¹ | (nM) | K_{D} (nM) |
| AB1424/AB1612 F3′ | 2.4 | 1.1 | 439.0 | 438.3 |
| TriNKET | | | | |
| AB1424/AB1612 F3′ | 2.4 | 1.1 | 452.9 | 453.0 |
| TriNKET | | | | |
| AB1424/AB1612 F3′ | 2.3 | 1.1 | 466.1 | 468.2 |
| TriNKET | | | | |
| AB1424/AB1612 F3′ | 2.4 | 1.1 | 465.1 | 466.3 |
| TriNKET | | | | |
| Average ± StDev | 2.4 ± 0.1 | 1.1 ± 0.0 | 455.8 ± 12.7 | 456.4 ± 13.9 |

TABLE 33
Kinetic parameters and binding affinities of AB1424/AB1612 F3′ TriNKET for
cynomolgus NKG2D by SPR.

| | | | 1:1 Kinetic Fit | Steady State Fit |
|---|---|---|---|---|
| Test article | kₐ (M⁻¹s⁻¹) × 10⁵ | k_{d} (s⁻¹) × 10⁻¹ | K_{D} (nM) | K_{D} (nM) |
| AB1424/AB1612 F3′ | 2.1 | 1.1 | 498.2 | 500.6 |
| TriNKET | | | | |
| AB1424/AB1612 F3′ | 2.0 | 1.0 | 515.8 | 516.4 |
| TriNKET | | | | |
| AB1424/AB1612 F3′ | 2.0 | 1.0 | 528.4 | 534.9 |
| TriNKET | | | | |
| AB1424/AB1612 F3′ | 2.1 | 1.1 | 525.4 | 530.3 |
| TriNKET | | | | |
| Average ± StDev | 2.0 ± 0.1 | 1.1 ± 0.0 | 517.0 ± 13.6 | 520.5 ± 15.5 |

### Binding to CD16

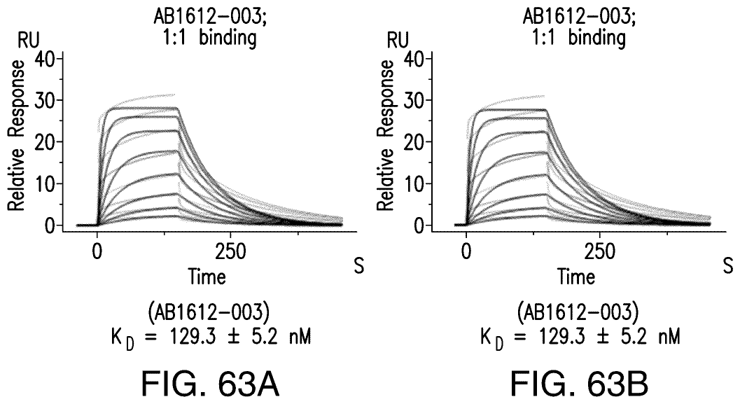

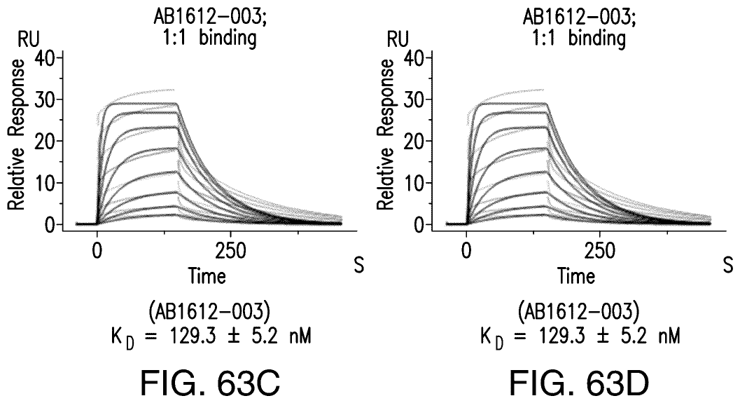

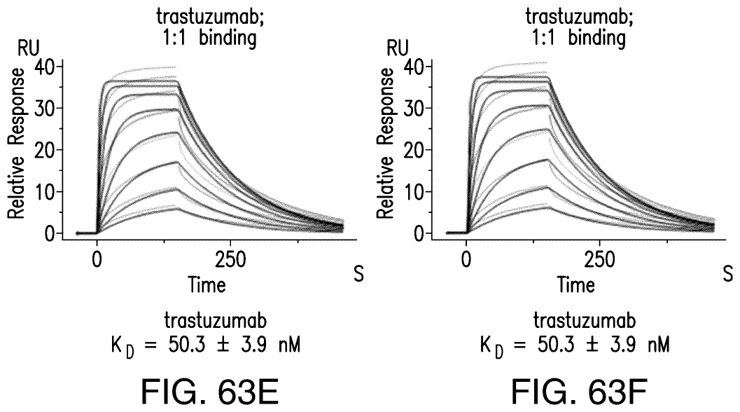

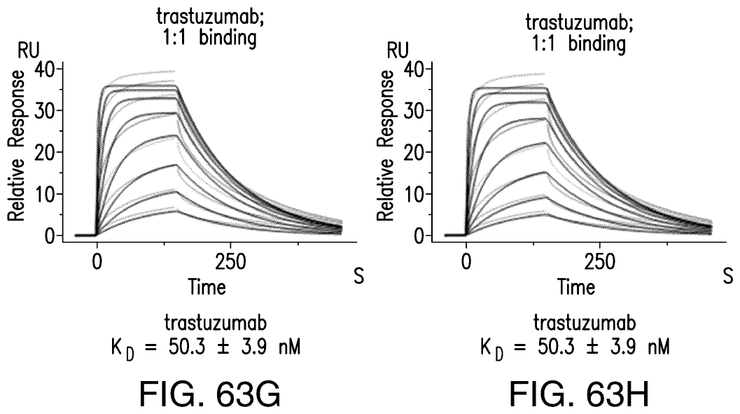

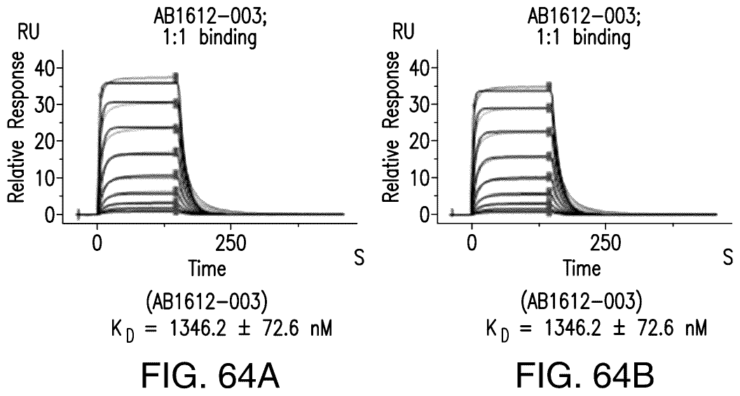

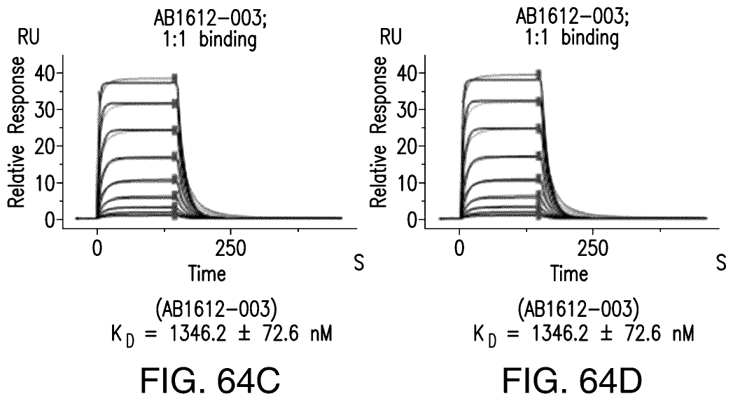

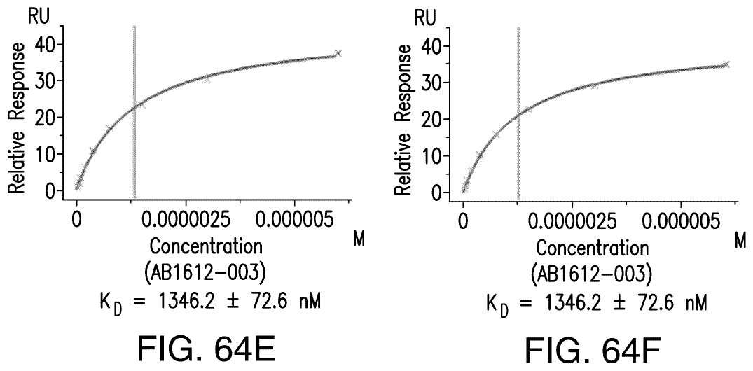

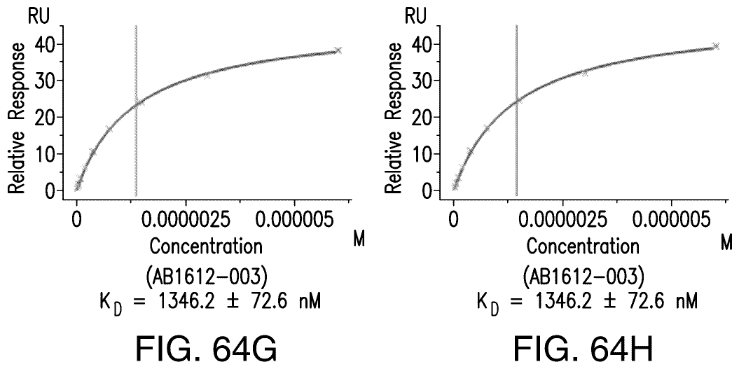

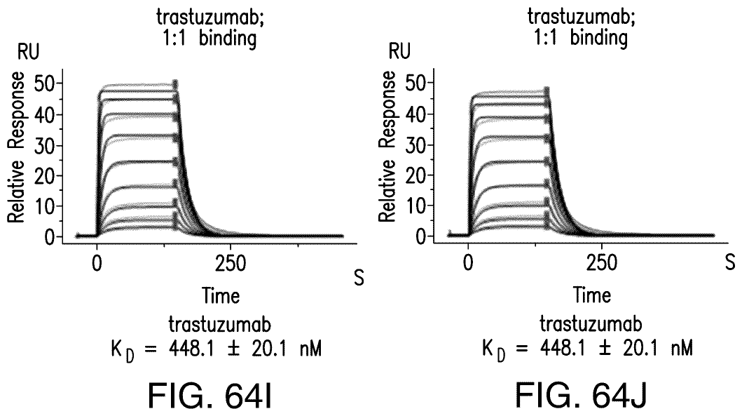

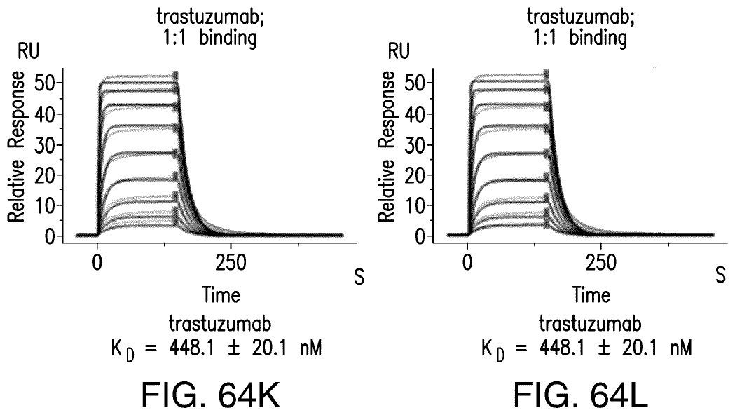

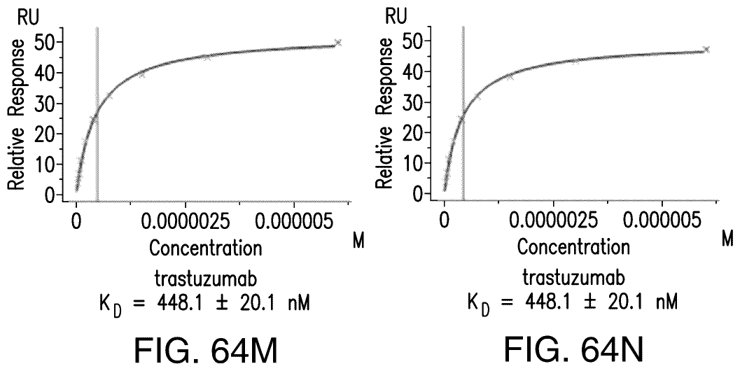

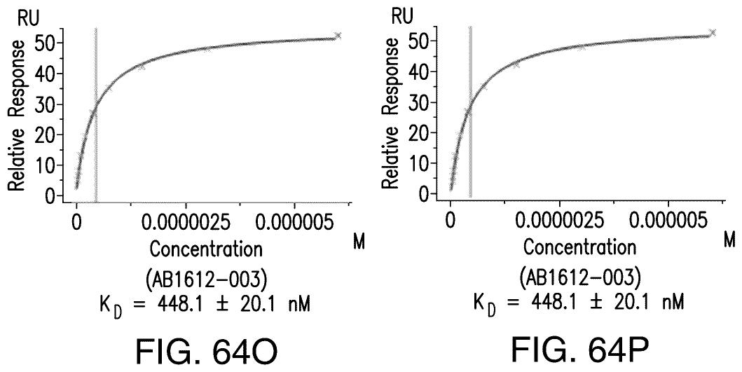

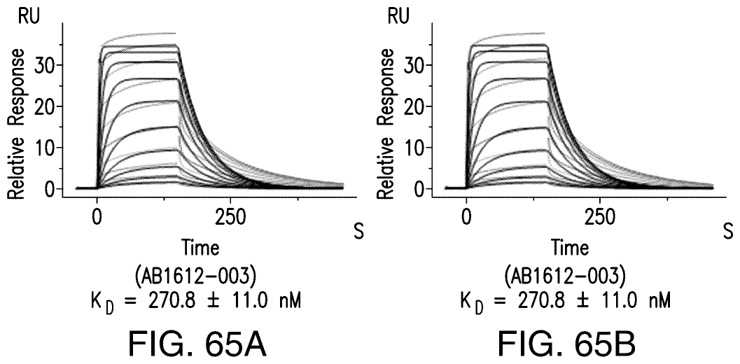

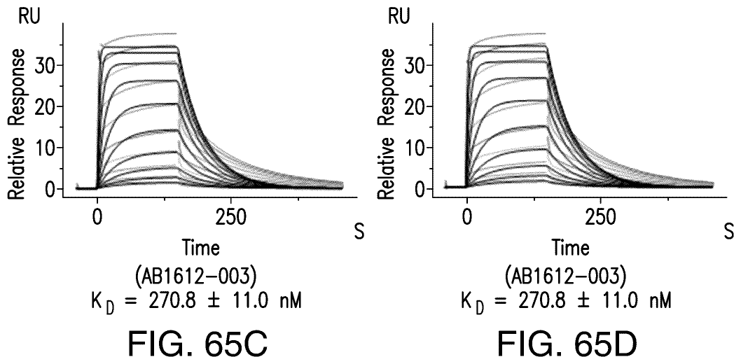

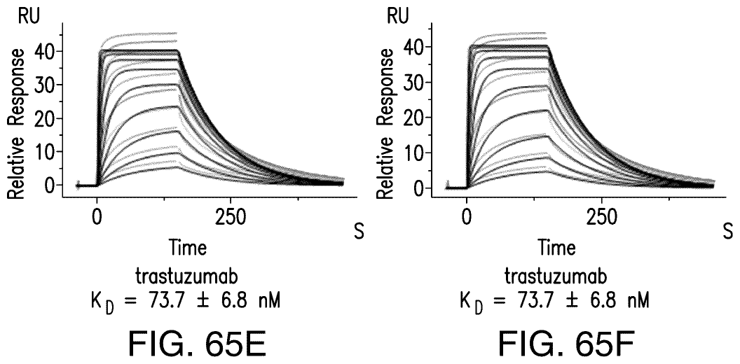

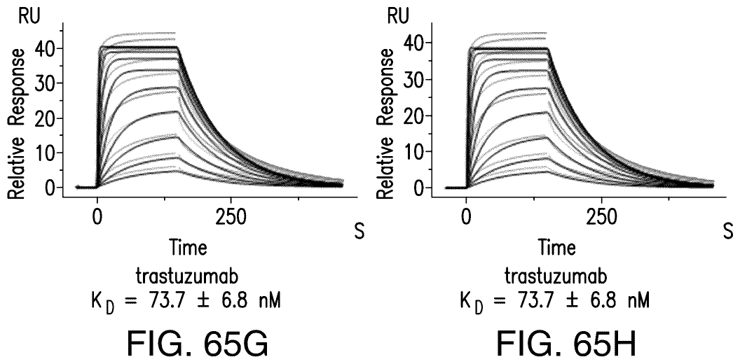

The binding of AB1424/AB1612 F3′ TriNKET to human CD16a (V158), human CD16a (F158) and cynomolgus CD16 was assessed by SPR and compared to trastuzumab (FIG. 63A-FIG. 63H, FIG. 64A-FIG. 64P, and FIG. 65A-FIG. 65H). The kinetics of human CD16a V158 engagement were comparable between AB1424/AB1612 F3′ TriNKET and IgG1 isotype experimental control trastuzumab (Table 34). Likewise, the steady state affinities for AB1424/AB1612 F3′ TriNKET and trastuzumab to human CD16a F158 were comparable (Table 35). Affinities to cynomolgus CD16 were comparable to human CD16a V158 for AB1424/AB1612 F3′ TriNKET and trastuzumab (Table 36). Therefore, AB1424/AB1612 F3′ TriNKET demonstrated favorable binding properties to human CD16a V158/F158 and cynomolgus CD16.

TABLE 34
Kinetic parameters and binding affinities of AB1424/AB1612 F3′
TriNKET 1 for human CD16a V158.

| | | | Kinetic |
|---|---|---|---|
| Test article | kₐ (M⁻¹s⁻¹) | k_{d} (s⁻¹) | Fit K_{D} (nM) |
| AB1424/AB1612 F3′ | 1.1 × 10⁵ | 1.4 × 10⁻² | 130.6 |
| TriNKET | | | |
| AB1424/AB1612 F3′ | 1.0 × 10⁵ | 1.4 × 10⁻² | 131.3 |
| TriNKET | | | |
| AB1424/AB1612 F3′ | 1.1 × 10⁵ | 1.4 × 10⁻² | 133.6 |
| TriNKET | | | |
| AB1424/AB1612 F3′ | 1.1 × 10⁵ | 1.3 × 10⁻² | 121.7 |
| TriNKET | | | |
| Average ± StDev | (1.1 ± 0.0) × 10⁵ | (1.4 ± 0.1) × 10⁻² | 129.3 ± 5.2 |
| trastuzumab | 1.9 × 10⁵ | 9.2 × 10⁻³ | 48.9 |
| trastuzumab | 1.9 × 10⁵ | 9.2 × 10⁻³ | 48.6 |
| trastuzumab | 1.9 × 10⁵ | 9.0 × 10⁻³ | 47.7 |
| trastuzumab | 1.6 × 10⁵ | 8.9 × 10⁻³ | 56.1 |
| Average ± StDev | (1.8 ± 0.2) × 10⁵ | (9.1 ± 0.2) × 10⁻³ | 50.3 ± 3.9 |

TABLE 35
Steady state affinity of AB1424/AB1612 F3' TriNKET
binding to human CD16a F158.

| Test article | Steady state K_{D} (nM) |
|---|---|
| AB1424/AB1612 F3' TriNKET | 1331.8 |
| AB1424/AB1612 F3' TriNKET | 1256.0 |
| AB1424/AB1612 F3' TriNKET | 1366.8 |
| AB1424/AB1612 F3' TriNKET | 1430.0 |
| Average ± StDev | 1346.2 ± 72.6 |
| trastuzumab | 475.7 |
| trastuzumab | 428.5 |
| trastuzumab | 447.9 |
| trastuzumab | 440.2 |
| Average ± StDev | 448.1 ± 20.1 |

TABLE 36
Kinetic parameters and binding affinities of
AB1424/AB1612 F3' TriNKET for cynomolgus CD16.

| | | | Kinetics Fit |
|---|---|---|---|
| Test article | kₐ (M⁻¹s−¹) | k_{d} (s⁻¹) | K_{D} (nM) |
| AB1424/AB1612 F3' | 7.8 × 10⁴ | 2.0 × 10⁻² | 232.5 |
| TriNKET | | | |
| AB1424/AB1612 F3' | 7.8 × 10⁴ | 2.1 × 10⁻² | 225.4 |
| TriNKET | | | |
| AB1424/AB1612 F3' | 7.2 × 10⁴ | 2.0 × 10⁻² | 238.3 |
| TriNKET | | | |
| AB1424/AB1612 F3' | 7.8 × 10⁴ | 2.0 × 10⁻² | 239.1 |
| TriNKET | | | |
| Average ± StDev | (7.6 ± 0.4) × 10⁴ | (2.1 ± 0.0) × 10⁻² | 270.8 ± 11.0 |
| trastuzumab | 1.9 × 10⁵ | 1.2 × 10⁻² | 66.1 |
| trastuzumab | 1.6 × 10⁵ | 1.2 × 10⁻² | 76.2 |
| trastuzumab | 1.6 × 10⁵ | 1.2 × 10⁻² | 78.9 |
| trastuzumab | 1.6 × 10⁵ | 1.3 × 10⁻² | 79.0 |
| Average ± StDev | (1.7± 0.2) × 10⁵ | (1.2 ± 0.0) × 10⁻² | 73.7 ± 6.8 |

### Co-Engagement of Antigen-Binding Sites

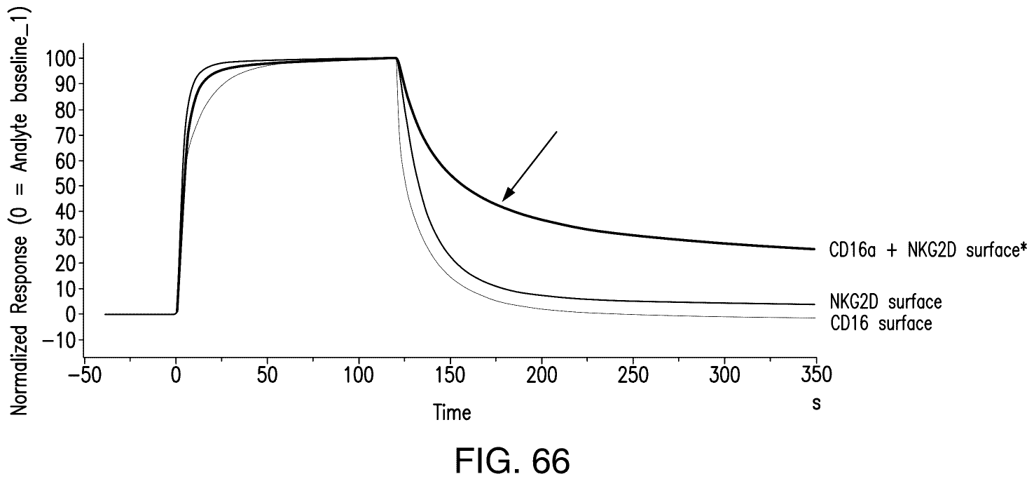

To demonstrate synergy of co-engagement of human CD16a and human NKG2D binding, an SPR experiment was performed where binding of AB1424/AB1612 F3′ TriNKET to NKG2D, CD16a and a mixed NKG2D-CD16a Biacore chip surfaces was qualitatively evaluated. The affinity of AB1424/AB1612 F3′ TriNKET for human NKG2D and human CD16a are both low, however, binding to both targets simultaneously results in an avidity effect that manifests as a slower off-rate. Thus, AB1424/AB1612 F3′ TriNKET can avidly engage CD16a and NKG2D (FIG. 66).

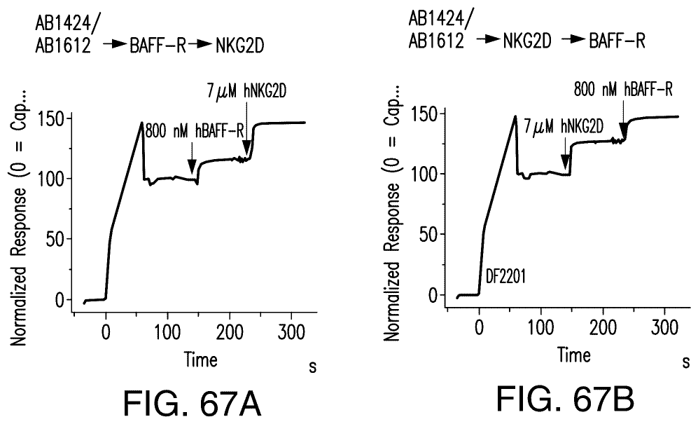

To determine if binding of one target interferes with binding of the second target to AB1424/AB1612 F3′ TriNKET, BAFF-R and NKG2D were sequentially injected over AB1424/AB1612 F3′ TriNKET that was captured on an anti-hFc IgG SPR chip. Target binding sensorgrams demonstrate that the occupancy status of the BAFF-R binding arm or the NKG2D binding arm following saturation does not interfere with association of the second target antigen (FIG. 67A and FIG. 67B). Similarity in shapes of the respective sensorgrams segments depicting binding of each target to free AB1424/AB1612 F3′ TriNKET and AB1424/AB1612 F3′ TriNKET that has been saturated with the other target suggests that the kinetic parameters are not meaningfully affected by the target occupancy status of the AB1424/AB1612 F3′ TriNKET. For instance, the shape of the BAFF-R binding segment of the sensorgrams is similar in both panels. A saturating concentration of the NKG2D had to be maintained throughout the entire experiment due to fast dissociation rate of this target. Additionally, the lack of any impact on relative stoichiometry of each target binding (when compared to binding to unoccupied AB1424/AB1612 F3′ TriNKET) signifies full independence of NKG2D and BAFF-R binding sites on AB1424/AB1612 F3′ TriNKET (Table 37). Therefore, AB1424/AB1612 F3′ TriNKET was able to successfully achieve simultaneous co-engagement of the BAFF-R and the NKG2D targeting arm.

TABLE 37
Relative binding stoichiometries of AB1424/AB1612 F3' TriNKET
for BAFF-R and NKG2D.

| | BAFF-R | NKG2D |
|---|---|---|
| | relative binding | relative binding |
| Experimental setup | stoichiometry | stoichiometry |
| Target Bound to AB1424/AB1612 F3' | 1.0 | 1.0 |
| TriNKET unoccupied with another target | | |
| (injected first) | | |
| Target Bound to AB1424/AB1612 F3' | 1.1 ± 0.1 | 1.0 ± 0.1 |
| TriNKET saturated with another target | | |
| (injected second) | | |

### Cell Binding Specificity

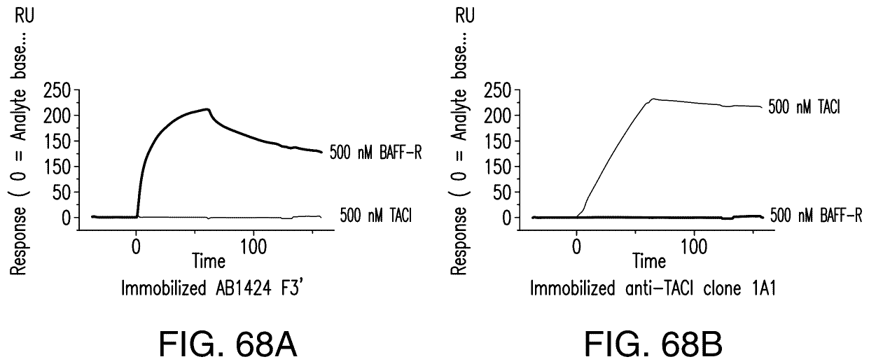

To assess specificity of AB1424/AB1612 F3′ TriNKET for BAFF-R, it was tested for binding to a closely-related proteins that also bind BAFF ligand. In FIG. 68A and FIG. 68B, an SPR experiment was performed and demonstrated that immobilized AB1424/AB1612 F3′ TriNKET binds specifically to target BAFF-R and not TACI. The activity and proper folding of the recombinant human TACI was confirmed by binding to the TACI-specific antibody (panel on the right).

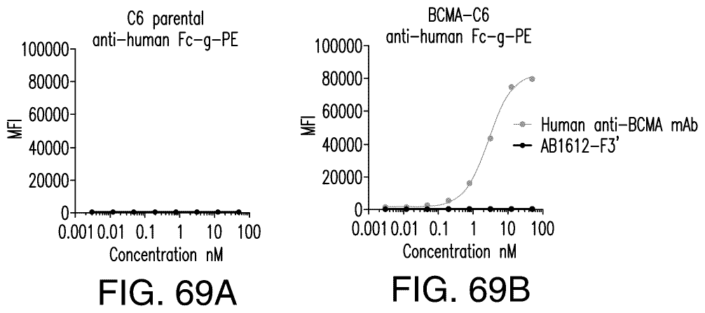

To further assess the specificity of AB1424/AB1612 F3′ TriNKET for BAFF-R, binding of AB1424/AB1612 F3′ TriNKET to a transgenic cell line expressing another BAFF binding family member BCMA (BCMA-C6) was assessed by flow cytometry (FIG. 69A and FIG. 69B). AB1424/AB1612 F3′ TriNKET did not show cross-reactivity to BCMA, or the rat-derived parental cell line C6. A mAb with known BCMA specificity (EM901) was used as a positive control for BCMA detection.

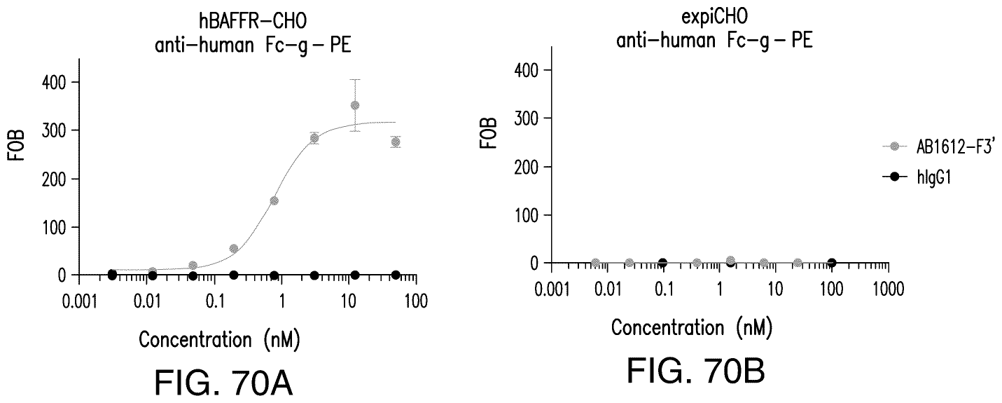

Specificity of AB1424/AB1612 F3′ TriNKET and lack of interactions with unrelated proteins were further assessed by probing binding to ExpiCHO isogenic cell lines engineered to express human or cynomolgus BAFF-R cells (FIG. 70A and FIG. 70B).

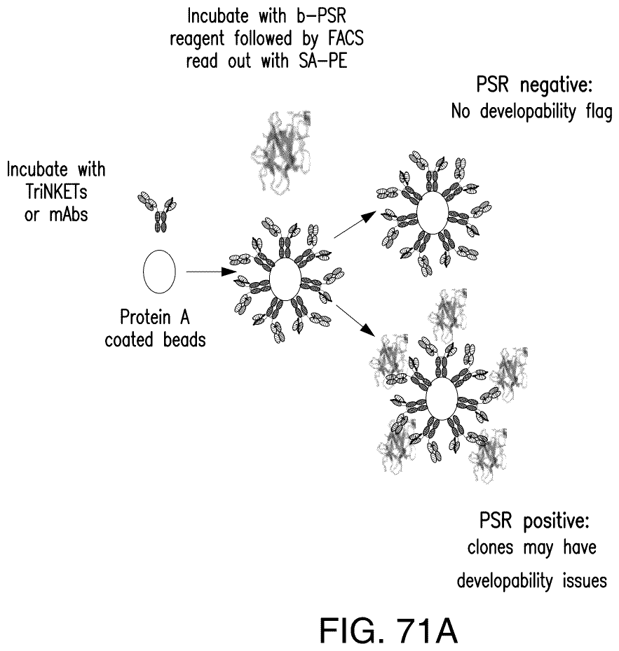

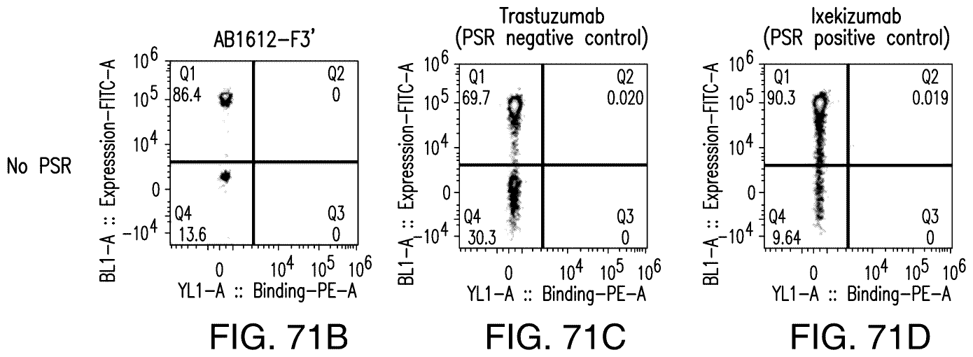

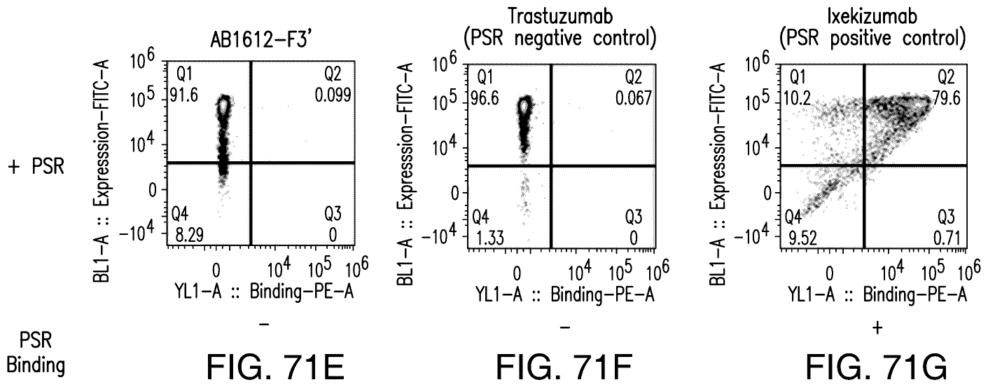

Additionally, a flow cytometry-based PSR assay to measure binding to a preparation of detergent solubilized CHO cell membrane proteins was performed (FIG. 71A-FIG. 71G). The PSR assay correlates well with cross-interaction chromatography, a surrogate for antibody solubility, as well as with baculovirus particle enzyme-linked immunosorbent assay, a surrogate for in vivo clearance (Xu et. al (2013). Addressing polyspecificity of antibodies selected from an in vitro yeast presentation system: a FACS-based, high-throughput selection and analytical tool. Protein engineering design and selection, 26, 663-670).

50 μL of 100 nM TriNKET or control mAb in PBSF were incubated with pre-washed 5 μL protein A Dynabeads™ slurry (Invitrogen, catalog #10001D) for 30 minutes at room temperature. TriNKET or mAb bound magnetic beads were allowed to stand on a magnetic rack for 60 seconds, and the supernatant was discarded. The bound beads were washed with 100 μL PBSF. Beads were incubated for 20 minutes on ice with 50 μL of biotinylated PSR reagent, which was diluted 25-fold from the stock (Xu et. al. 2013). Samples were put on the magnetic rack, supernatant discarded, and washed with 100 μL of PBSF. A secondary FACS reagent, to detect binding of biotinylated PSR reagent to TriNKETs or control mAbs, was made as follows: 1:250 μL of Streptavidin-PE (Biolegend, catalog #405204) and 1:100 donkey anti-human Fc were combined in PBSF. To each sample, 100 μL of the secondary reagents were added and allowed to incubate for 20 minutes on ice. The beads were washed twice with 100 μL PBSF, and samples were analyzed on a FACS Celesta (BD). In this assay, trastuzumab served as a negative control. Ixekizumab served as positive control, with increasing propensity for interaction with PSR by flow cytometry. AB1424/AB1612 F3′ TriNKET was negative for binding to PSR and was most comparable to the PSR negative control, trastuzumab. These results indicate that AB1424/AB1612 F3′ TriNKET does not exhibit reactivity with non-specific proteins (FIG. 71A-FIG. 71G).

### KHYG1-CD16aV Mediated Cytotoxicity

The potency of AB1424/AB1612 F3′ TriNKET in stimulating KHYG-1-CD16aV mediated cytolysis of BAFF-R+ BJAB cells was determined in a cytotoxicity assay using KHYG-1-CD16a cells engineered to express CD16a in addition to NKG2D. Lysis of target cells was measured by the DELFIA cytotoxicity assay. Briefly, human cancer cell lines expressing BAFF-R were harvested from culture, washed with HBS, and resuspended in growth media at 10⁶/mL for labeling with BATDA reagent (Perkin Elmer AD0116). Manufacturer instructions were followed for labeling of the target cells. After labeling, cells were washed three times with HBS and resuspended at 0.5-1.0×10⁵/mL in culture media. 100 μl of BATDA-labeled cells were added to each well of the 96-well plate. Monoclonal antibodies or TriNKETs against BAFF-R were diluted in culture media, and 50 μl of diluted mAb or TriNKET were added to each well.

To prepare NK cells, PBMCs were isolated from human peripheral blood buffy coats using density gradient centrifugation, washed, and prepared for NK cell isolation. NK cells were isolated using a negative selection technique with magnetic beads. Purity of isolated NK cells was typically >90% CD3-CD56+. Isolated NK cells were rested overnight and harvested from culture. The cells were then washed and resuspended at concentrations of 10⁵-2.0×10⁶/mL in culture media for an effector-to-target (E:T) ratio of 5:1. 50 μl of NK cells were added to each well of the plate for a total of 200 μl culture volume. The plate was incubated at 370 C with 5% CO₂ for 2-3 hours.

After the incubation, the plate was removed from the incubator, and the cells were pelleted by centrifugation at 200×g for 5 minutes. 20 μl of culture supernatant were transferred to a clean microplate and 200 μl of room temperature europium solution (Perkin Elmer C135-100) were added to each well. The plate was protected from light and incubated on a plate shaker at 250 rpm for 15 minutes, then read using SpectraMax i3X instruments. BJAB cells were labeled with BATDA reagent. After labeling, cells were washed and resuspended in primary cell culture media. BATDA labeled cells, AB1424/AB1612 F3′ TRINKET, and rested KHYG-1-CD16V cells were added to the wells of a 96-well plate. Additional wells were prepared for maximum lysis of target cells by addition of 1% Triton-X. Spontaneous release was monitored from wells with only BATDA-labeled cells. After three hours of culture, the cells were pelleted, the culture supernatant was transferred to a clean microplate, and room temperature europium solution was added to each well. The plate was protected from light and incubated on a plate-shaker at 250 rpm for 15 minutes. Plates were read using a SpectraMax i3X instrument. The % Specific lysis was calculated as follows:

% Specific lysis=((Experimental release−Spontaneous release)/(Maximum release−Spontaneous release))*100%

AB1424/AB1612 F3′ TriNKET exhibited comparable sub-nanomolar potency and effective maximum cell killing (Table 38). AB1424/AB1612 F3′ TriNKET was highly potent in driving the lysis of BJAB cells, and there was strong correlation of potency across production lots of AB1424/AB1612 F3′ TriNKET.

TABLE 38
Potency of AB1424/AB1612 F3' TriNKET in the
presence of KHYG-1-CD16aV and
BJAB cells. Max killing is presented relative to control.

| Test article | EC₅₀ (nM) | Max killing (%) |
|---|---|---|
| AB1424/AB1612 F3' TriNKET | 0.13 | 108 |
| AB1424/AB1612 F3' TriNKET | 0.13 | 105 |

### NK-Cell Mediated Cytotoxicity

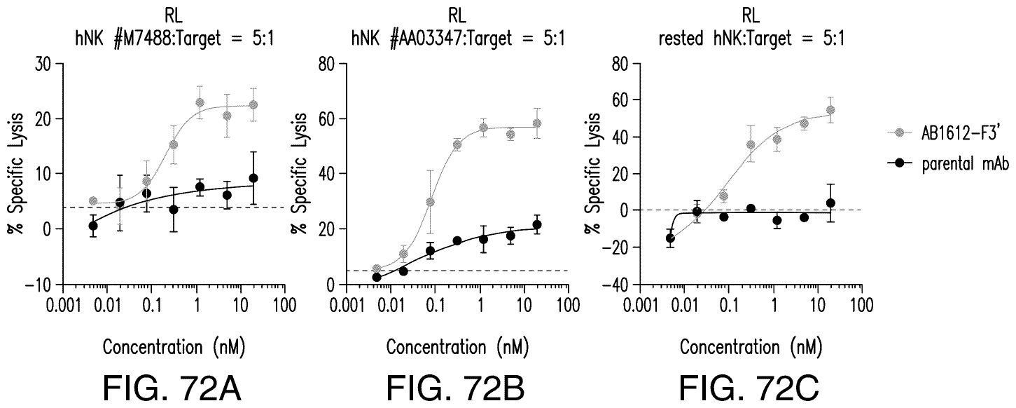

The potency of AB1424/AB1612 F3′ TriNKET in driving the NK cell-mediated lysis of BAFF-R+ tumor cell line RL was compared to the parental AB1753 antibody (FIG. 72A and FIG. 72B). The cytotoxicity assay was performed as described in Example 2. AB1753 elicited low or no detectable cytolysis of BAFF-R+ tumor cell line. AB1424/AB1612 F3′ TriNKET demonstrated a sub-nanomolar EC₅₀, efficient maximum killing, and exceeded the potency of AB1753 in the lysis of RL cells (Table 39).

TABLE 39
Potency of AB1424/AB1612 F3' TriNKET and parental mAb in the
presence of primary NK cells and BAFF-R+ tumor cell line.

| | AB1424/AB1612 F3' TriNKET | | AB1753 | |
|---|---|---|---|---|
| | | Max | EC₅₀ | Max |
| Cell line | EC₅₀ (nM) | killing (%) | (nM) | killing (%) |
| RL | 0.12 ± 0.07 | 44.3 ± 19.2 | ND | 10.7 ± 9.6 |

### Potency of AB1424/AB1612 F3′ TriNKET Requires Both NKG2D and CD16a Engagement

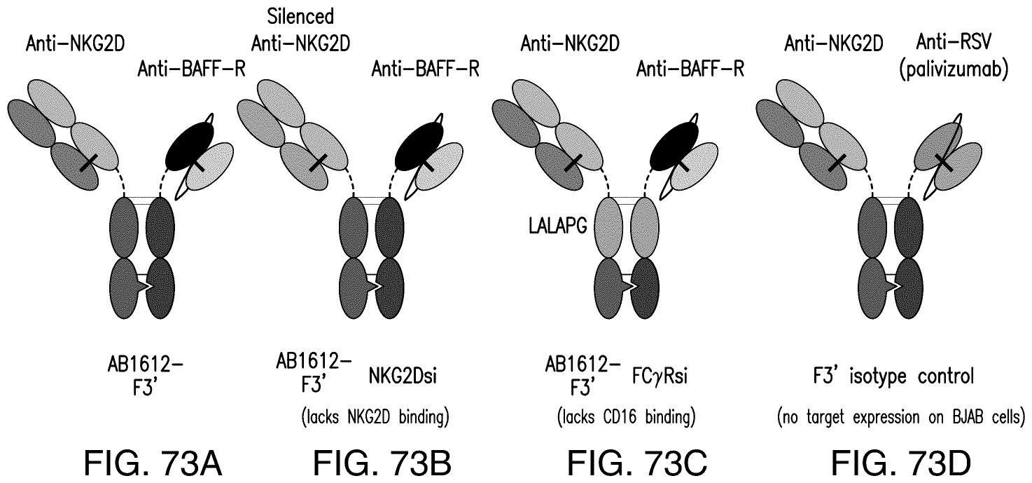

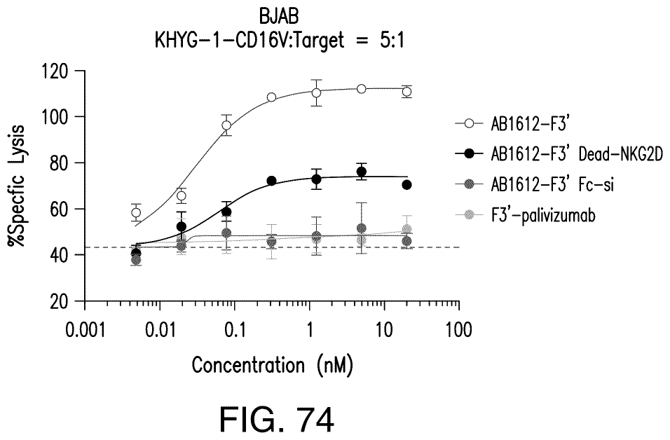

To evaluate the mechanism of AB1424/AB1612 F3′ TriNKET-mediated cytotoxicity, a panel of control TriNKETs was generated. AB1424/AB1612 F3′ TRINKET NKG2Dsi is a variant of AB1424/AB1612 F3′ TriNKET where the light chain of the NKG2D-binding arm was substituted, rendering the arm incapable of binding to NKG2D. AB1424/AB1612 F3′ TriNKET FcγRsi is an effector-silenced version of AB1424/AB1612 F3′ TriNKET, bearing the Fc silencing mutations: L234A, L235A, and P329G (according to EU numbering). The F3′ isotype control was constructed by substituting the BAFF-R-binding arm with the variable domain of palivizumab, which binds to non-human antigen, formatted as a disulfide-stabilized scFv (FIG. 73A-FIG. 71D). Silenced variants behaved as expected, depending on their intended purpose, as qualitatively measured by SPR binding to human BAFF-R, NKG2D, and CD16a V158 (Table 40). AB1424/AB1612 F3′ TriNKET exhibited superior potency and maximal killing in the presence of KHYG-1-CD16a and BAFF-R+ BJAB; NKG2D, and Fc-silenced variants displayed minimal cytolytic activity (FIG. 74 and Table 41).

TABLE 41
Potency of AB1424/AB1612 F3' TriNKET and silenced variants
in the presence of KHYG-1-CD16a and BJAB cells.

| Test article | EC₅₀ (nM) | Max killing (%) |
|---|---|---|
| AB1424/AB1612 F3' TRINKET | 0.03 | 112 |
| AB1424/AB1612 F3' TRINKET | 0.06 | 74 |
| NKG2Dsi | | |
| AB1424/AB1612 F3' TRINKET | N.D. | 48 |
| FcγRsi | | |
| F3' isotype control | N.D. | 48 |

As described above, AB1424/AB1612 F3′ TriNKET bound with high affinity to human and cynomolgus BAFF-R, with low affinity to human and cynomolgus NKG2D, and with low affinity to human and cynomolgus CD16a. The AB1424/AB1612 F3′ TriNKET did not display any spurious off-target interactions. AB1424/AB1612 F3′ TriNKET bound tightly to and was highly potent against BAFF-R+ cells. Finally, AB1424/AB1612 F3′ TriNKET could simultaneously bind to BAFF-R and NKG2D and exhibit robust synergy between the NK engager arms, and its efficacy required tripartite binding to BAFF-R, NKG2D, and CD16a, highlighting the mechanism of action of the TriNKET.

### Example 5—Further Analysis of AB1424/AB1612 F3′ TriNKET Binding to CD16 Receptors

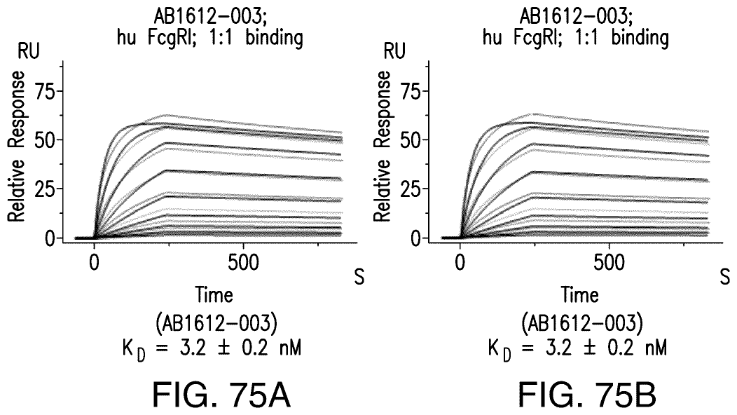

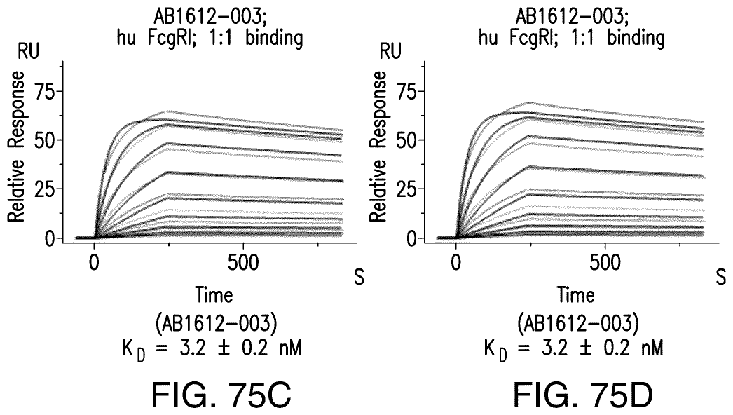

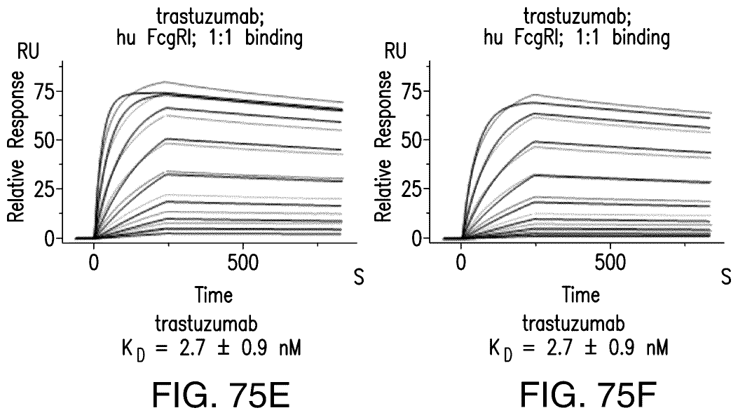

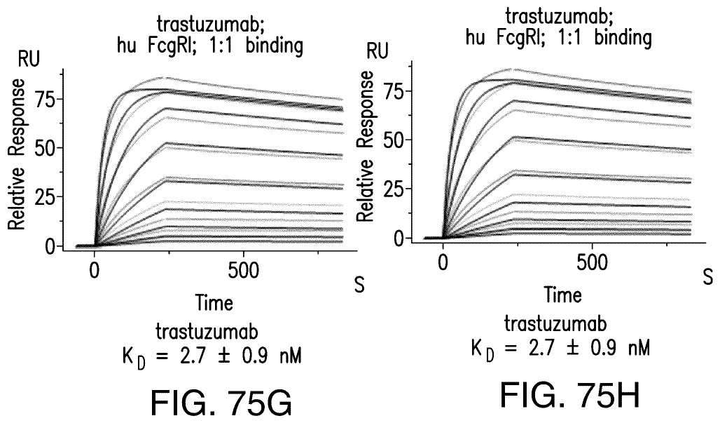

Binding analyses as described in this Example were performed using SPR, as described in Example 4. Binding of AB1424/AB1612 F3′ TriNKET to human CD64 (FcγRI) was measured and is shown in FIG. 75A-FIG. 75H. Table 42 summarizes kinetic rates and human CD64 affinity values determined from the sensorgrams for AB1424/AB1612 F3′ TriNKET and trastuzumab.

TABLE 42
Kinetic parameters and affinity values of AB1424/AB1612 F3'
TriNKET binding to human CD64.

| Sample | kₐ (1/Ms) | k_{d}(1/s) | K_{D} (nM) |
|---|---|---|---|
| AB1424/AB1612 | 7.7 × 10⁴ | 2.2 × 10⁻⁴ | 2.9 |
| F3'TRINKET | | | |
| AB1424/AB1612 | 7.3 × 10⁴ | 2.3 × 10⁻⁴ | 3.1 |
| F3'TRINKET | | | |
| AB1424/AB1612 | 6.9 × 10⁴ | 2.3 × 10⁻⁴ | 3.4 |
| F3'TRINKET | | | |
| AB1424/AB1612 | 7.2 × 10⁴ | 2.3 × 10⁻⁴ | 3.2 |
| F3'TRINKET | | | |
| Average ± SD | (7.3 ± 0.3) × 10⁴ | (2.3 ± 0.0) × 10⁻⁴ | 3.2 ± 0.2 |
| trastuzumab | 9.9 × 10⁴ | 2.0 × 10⁻⁴ | 2.0 |
| trastuzumab | 5.2 × 10⁴ | 2.0 × 10⁻⁴ | 3.9 |
| trastuzumab | 9.2 × 10⁴ | 2.1 × 10⁻⁴ | 2.2 |
| trastuzumab | 8.8 × 10⁴ | 2.2 × 10⁻⁴ | 2.5 |
| Average ± SD | (8.3 ± 2.1) × 10⁵ | (2.1 ± 0.1) × 10⁻⁴ | 2.7 ± 0.9 |

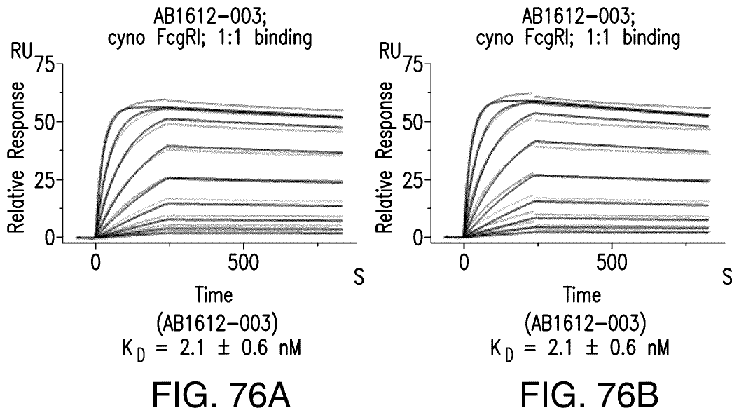

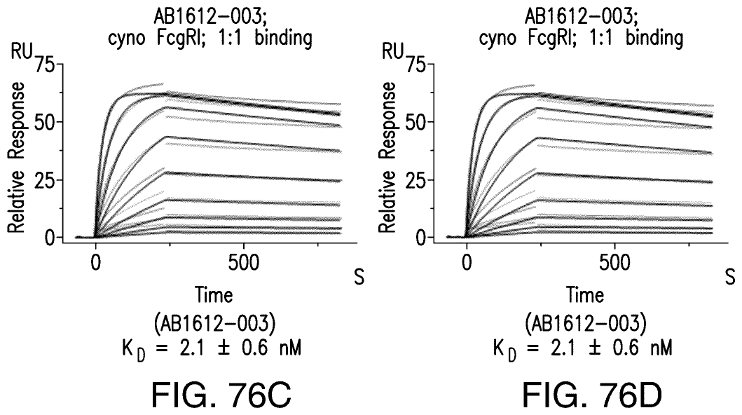

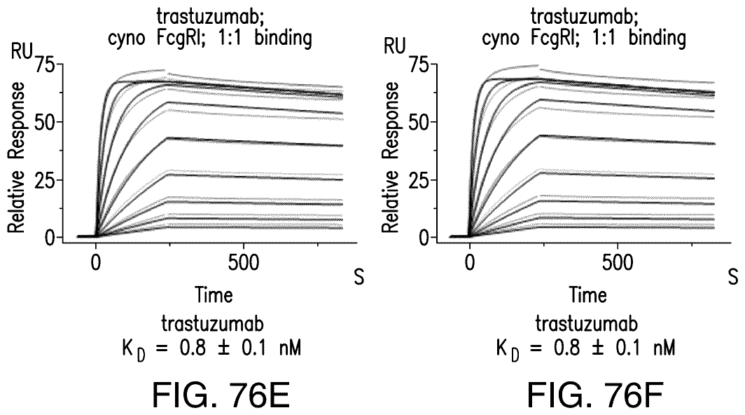

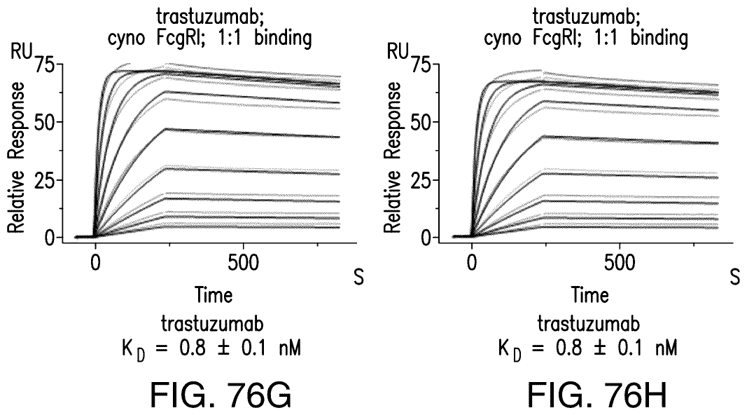

Binding of AB1424/AB1612 F3′ TriNET to cynomolgus CD64 (FcγRI) was measured and is shown in FIG. 76A-FIG. 76H. Table 43 summarizes kinetic rates and cynomolgus CD64 affinity values determined from the sensorgrams for AB1424/AB16121F3′ TriNKET and trastuzumab.

TABLE 43
Kinetic parameters and affinity values of AB1424/AB1612 F3'
TriNKET binding to cynomolgus CD64.

| Sample | kₐ (1/Ms) | k_{d}(1/s) | K_{D} (nM) |
|---|---|---|---|
| AB1424/AB1612 | 1.0 × 10⁵ | 1.3 × 10⁻⁴ | 1.3 |
| F3'TriNKET | | | |
| AB1424/AB1612 | 1.0 × 10⁵ | 1.9 × 10⁻⁴ | 1.9 |
| F3'TriNKET | | | |
| AB1424/AB1612 | 1.0 × 10⁵ | 2.5 × 10⁻⁴ | 2.4 |
| F3'TriNKET | | | |
| AB1424/AB1612 | 1.0 × 10⁵ | 2.7 × 10⁻⁴ | 2.7 |
| F3'TriNKET | | | |
| Average ± SD | (1.0 ± 0.0) × 10⁵ | (2.1 ± 0.7) × 10⁻⁴ | 2.1 ± 0.6 |
| trastuzumab | 1.7 × 10⁵ | 1.4 × 10⁻⁴ | 0.8 |
| trastuzumab | 1.8 × 10⁵ | 1.5 × 10⁻⁴ | 0.8 |
| trastuzumab | 1.8 × 10⁵ | 1.4 × 10⁻⁴ | 0.9 |
| trastuzumab | 1.8 × 10⁵ | 1.2 × 10⁻⁴ | 0.9 |
| Average ± SD | (1.8 ± 0.0) × 10⁵ | (1.4 ± 0.1) × 10⁻⁴ | 0.8 ± 0.1 |

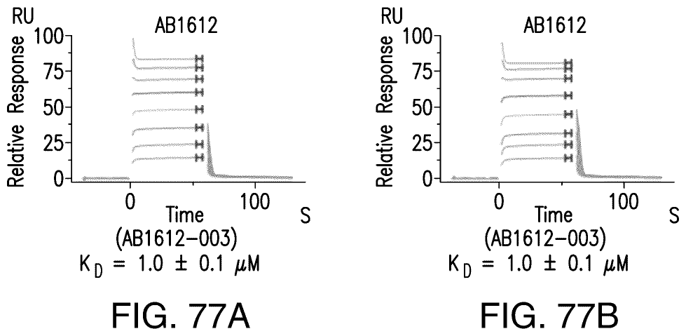

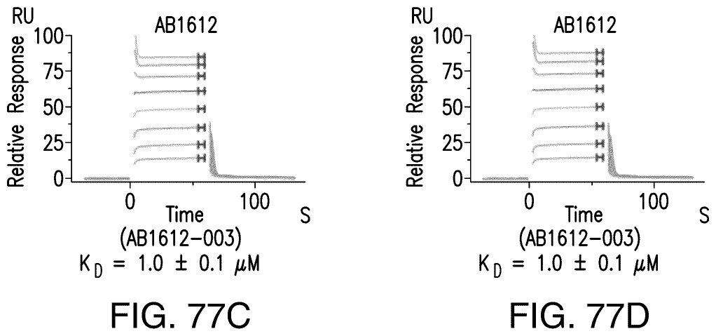

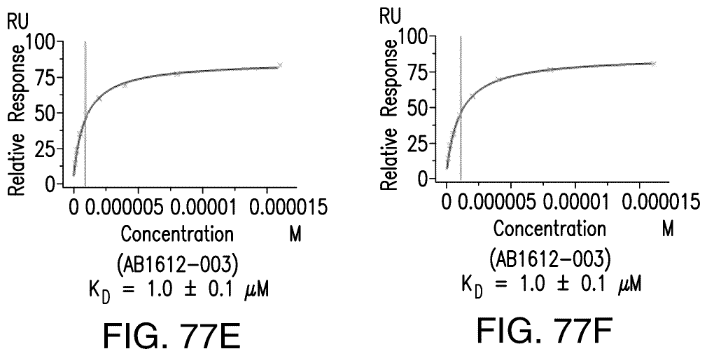

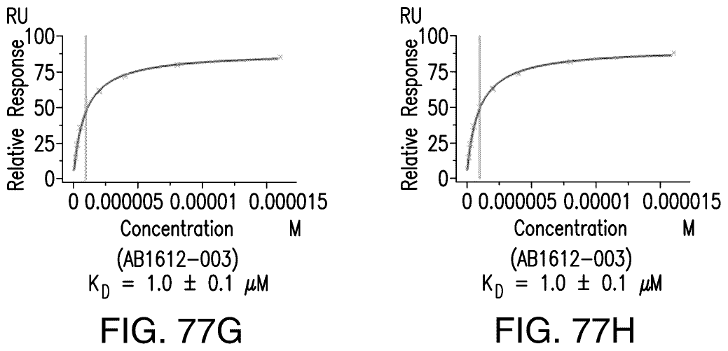

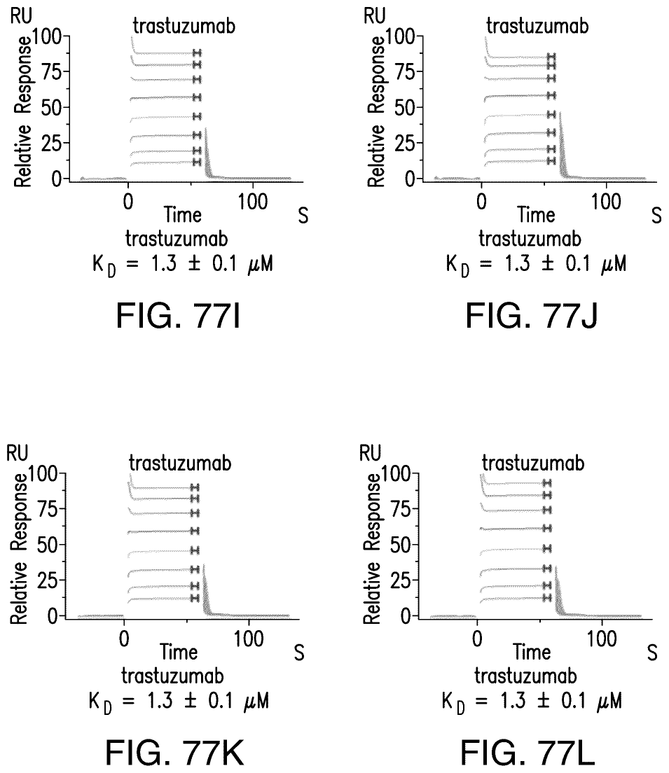

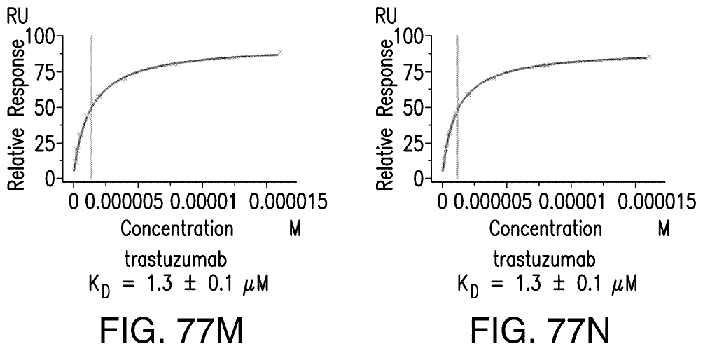

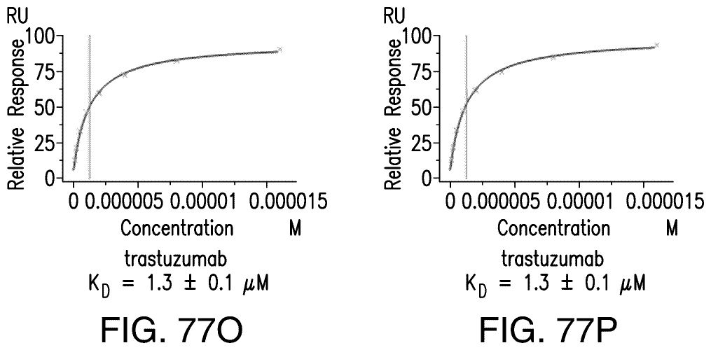

Binding to human CD32a H131 was measured and is shown in FIG. 77A-FIG. 77P. Affinity values determined from the sensorgrams are summarized in Table 44.

TABLE 44
Affinity values of AB1424/AB1612 F3' TriNKET and
trastuzumab for human CD32a H131.

| | Rep- | Rep- | Rep- | Rep- | K_{D} (μM) |
|---|---|---|---|---|---|
| | licate | licate | licate | licate | Average ± |
| Sample | K_{D} (μM) | K_{D} (μM) | K_{D} (μM) | K_{D} (μM) | SD |
| AB1424/AB16 | 0.9 | 1.1 | 0.9 | 1.0 | 1.0 ± 0.1 |
| 12 F3' | | | | | |
| TriNKET | | | | | |
| trastuzumab | 1.4 | 1.1 | 1.3 | 1.3 | 1.3 ± 0.1 |

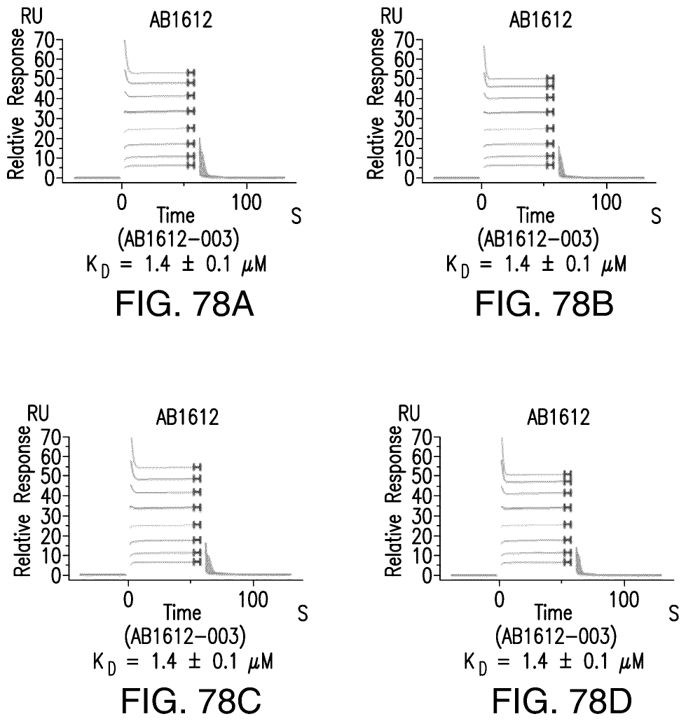

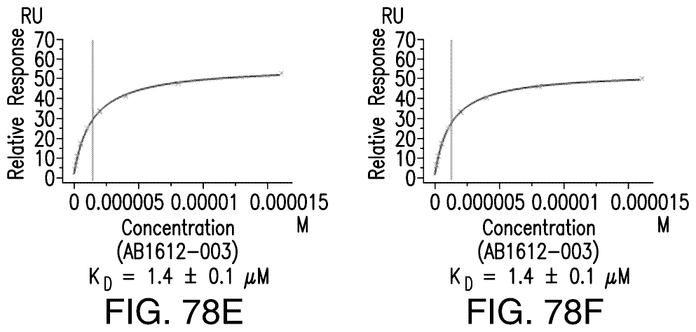

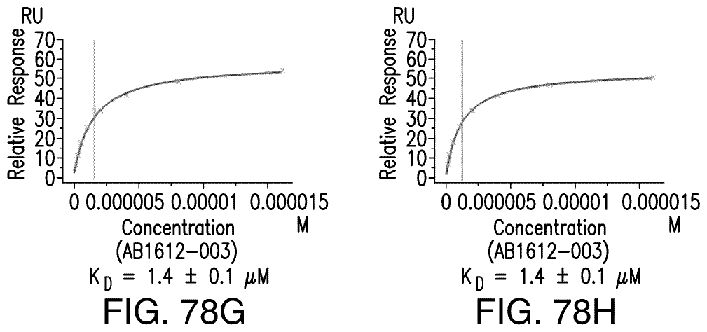

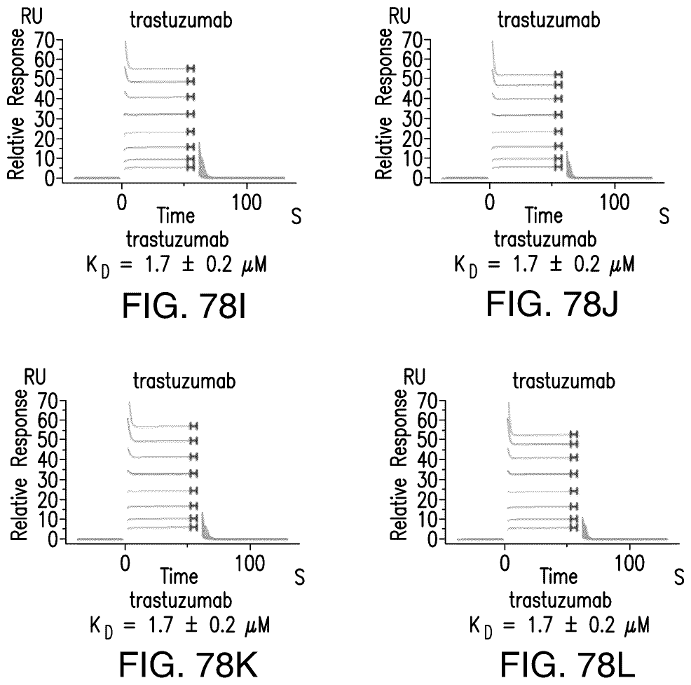

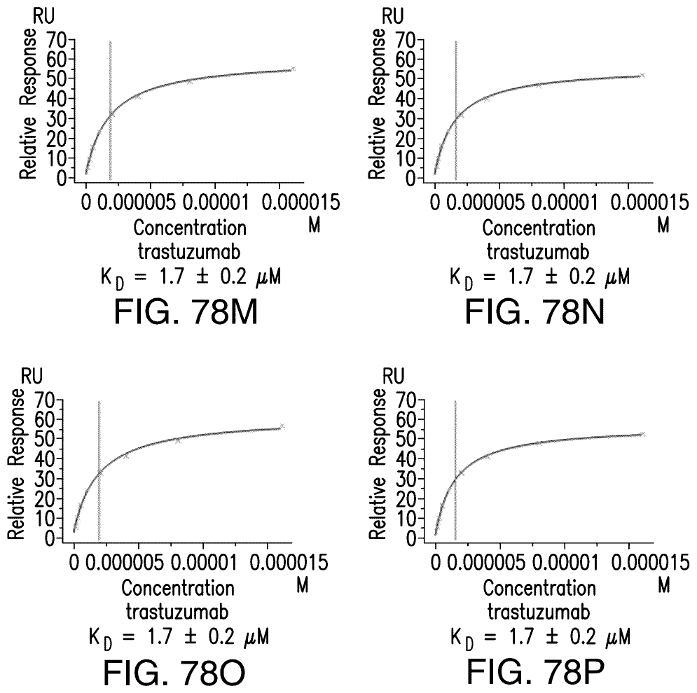

Binding of AB1424/AB1612 F3′ TriNKET to human CD32a R131 allele (FcγRIJa R131) was measured and is shown in FIG. 78A-FIG. 78P. The resulting affinity values determined from the sensorgrams are summarized in Table 45.

TABLE 45
Affinity values of AB1424/AB1612 F3' TriNKET and
trastuzumab for human CD32a R131.

| | Rep- | Rep- | Rep- | Rep- | K_{D} (μM) |
|---|---|---|---|---|---|
| | licate | licate | licate | licate | Average ± |
| Sample | K_{D} (μM) | K_{D} (μM) | K_{D} (μM) | K_{D} (μM) | SD |
| AB1424/AB16 | 1.5 | 1.3 | 1.5 | 1.2 | 1.4 ± 0.1 |
| 12 F3' | | | | | |
| TriNKET | | | | | |
| trastuzumab | 1.9 | 1.6 | 1.9 | 1.5 | 1.7 ± 0.2 |

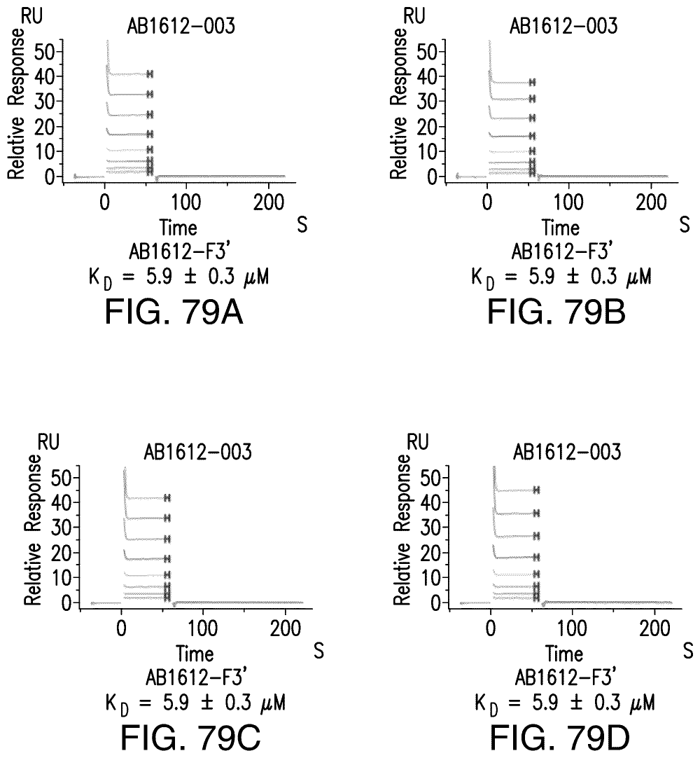

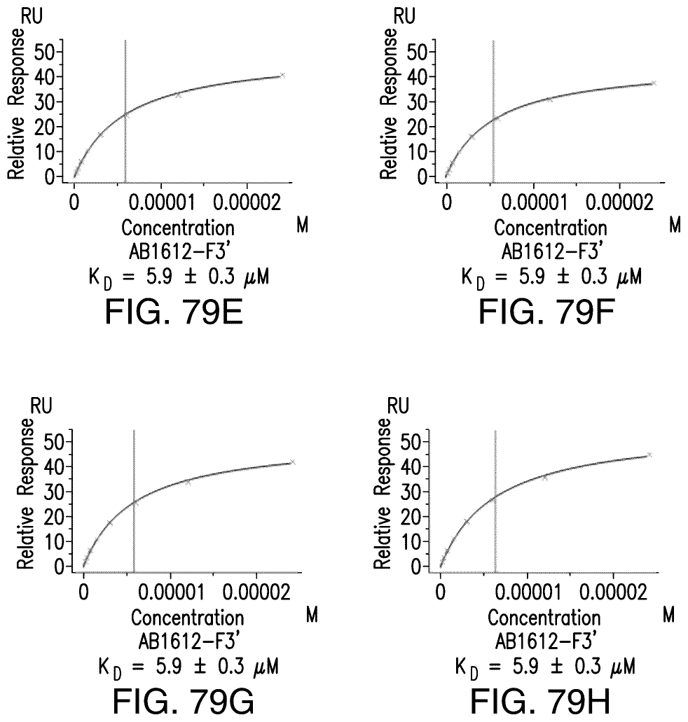

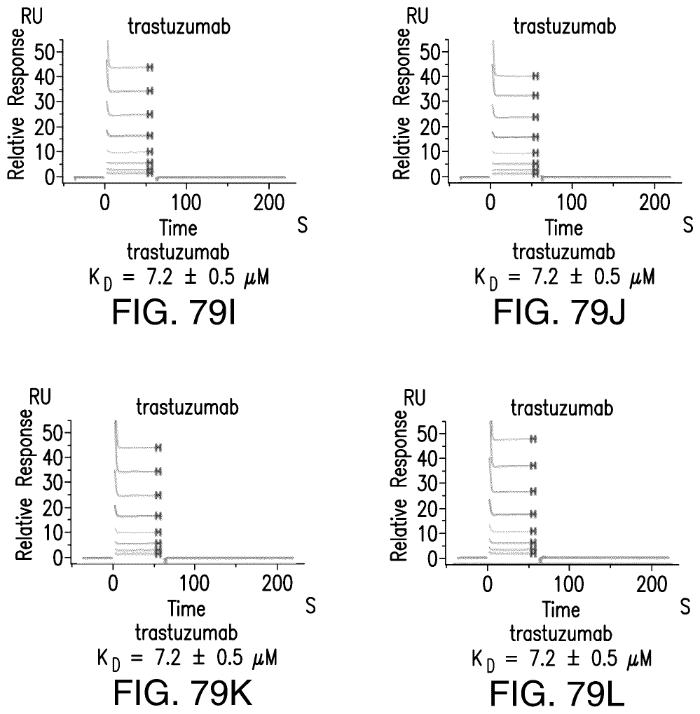

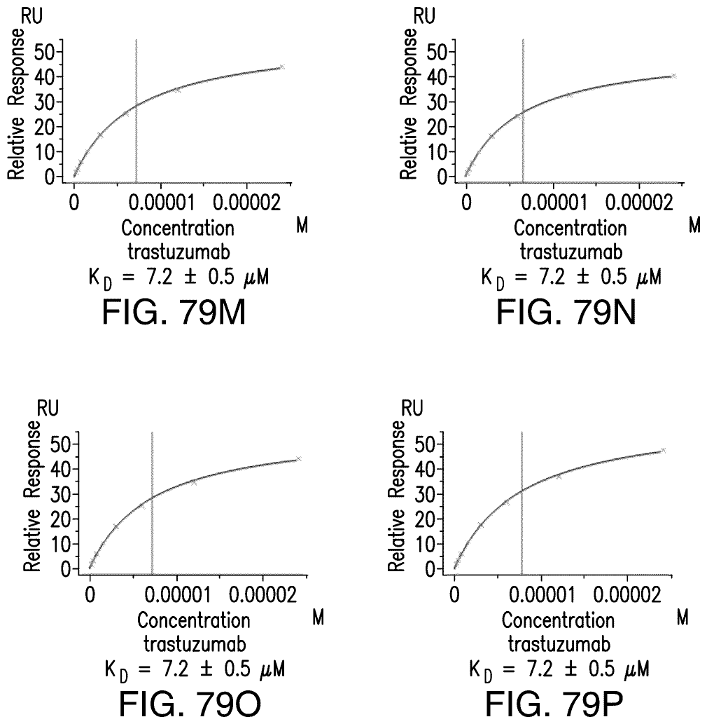

Binding of AB1424/AB1612 F3′ TriNKET to human CD32b (FcγRIIb) was measured and is shown in FIG. 79A-FIG. 79P. The resulting affinity values determined from the sensorgrams are summarized in Table 46.

TABLE 46
Affinity values of human CD32b for AB1424/AB1612 F3'
TriNKET and trastuzumab.

| | Rep- | Rep- | Rep- | Rep- | K_{D} (μM) |
|---|---|---|---|---|---|
| | licate | licate | licate | licate | Average ± |
| Sample | K_{D} (μM) | K_{D} (μM) | K_{D} (μM) | K_{D} (μM) | SD |
| AB1424/AB1 | 5.9 | 5.5 | 5.8 | 6.3 | 5.9 ± 0.3 |
| 612 F3' | | | | | |
| TriNKET | | | | | |
| trastuzumab | 7.2 | 6.6 | 7.2 | 7.7 | 7.2 ± 0.5 |

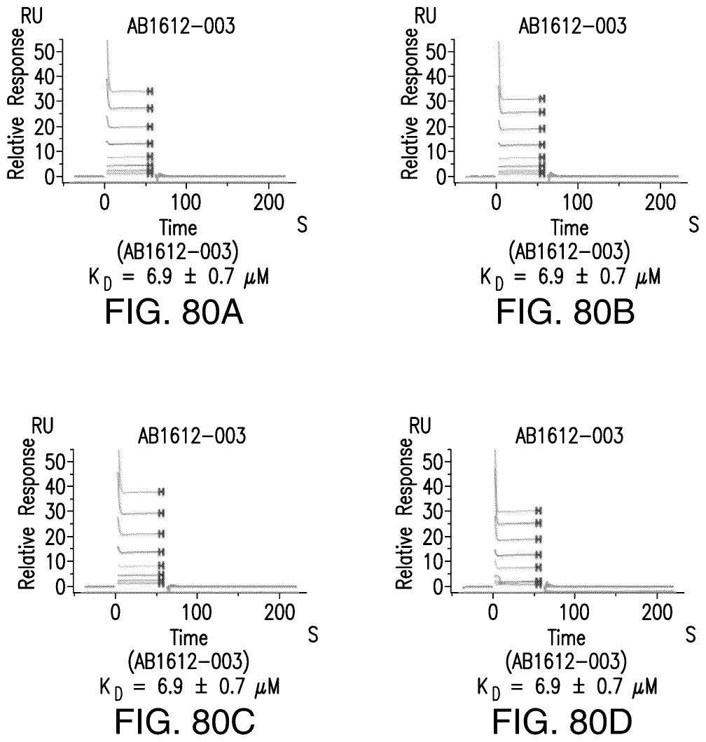

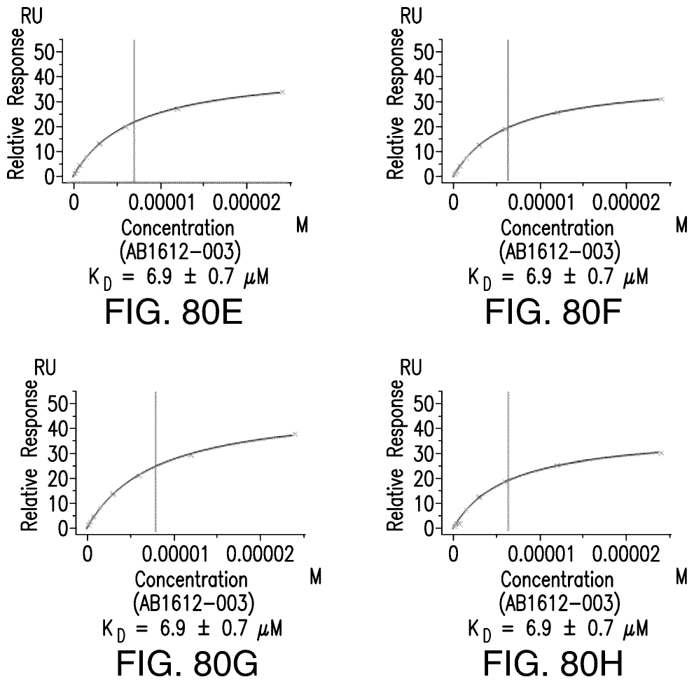

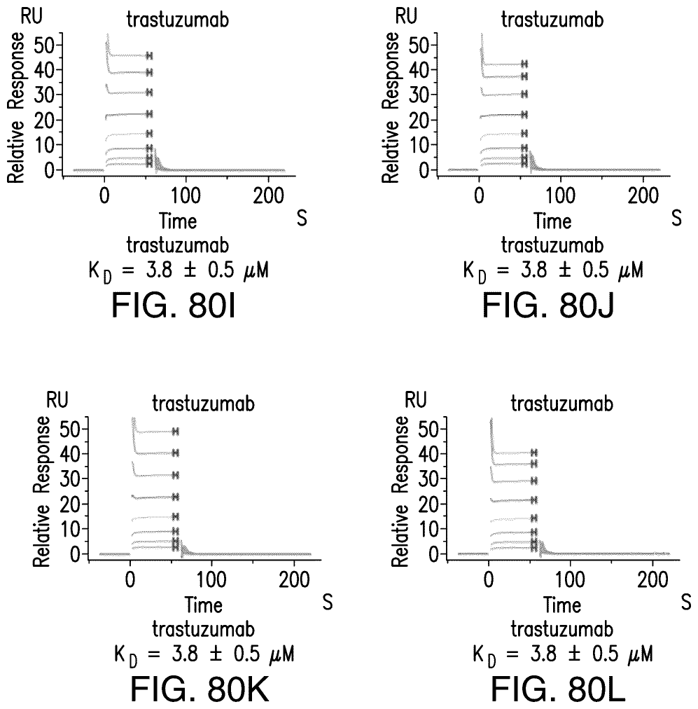

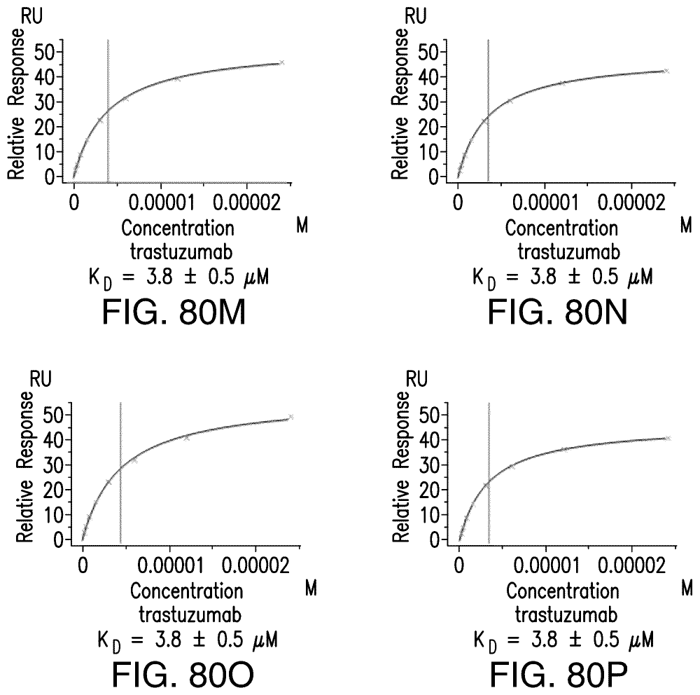

Binding of AB1424/AB1612 F3′ TriNKET to human CD16b (FcγRIIIb) was measured and is shown in FIG. 80A-FIG. 80P. The resulting affinity values determined from the sensorgrams are summarized in Table 47.

TABLE 47
Affinity values of human CD16b for AB1424/AB1612 F3'
TriNKET and trastuzumab.

| | Rep- | Rep- | Rep- | Rep- | K_{D} (μM) |
|---|---|---|---|---|---|
| | licate | licate | licate | licate | Average ± |
| Sample | K_{D} (μM) | K_{D} (μM) | K_{D} (μM) | K_{D} (μM) | SD |
| AB1424/AB1 | 7.0 | 6.3 | 7.9 | 6.4 | 6.9 ± 0.7 |
| 612 F3' | | | | | |
| TriNKET | | | | | |
| trastuzumab | 4.0 | 3.5 | 4.4 | 3.4 | 3.8 ± 0.5 |

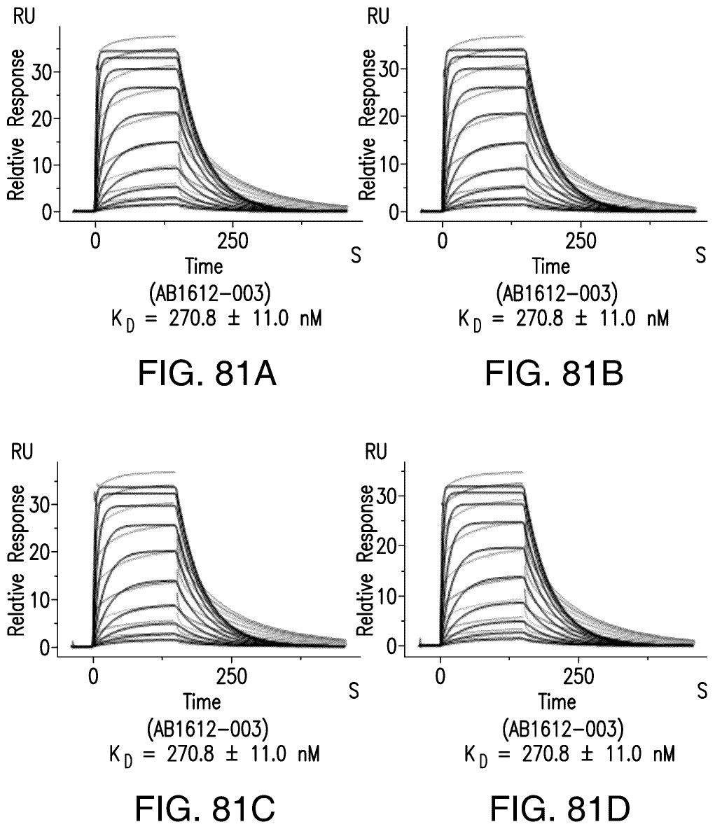

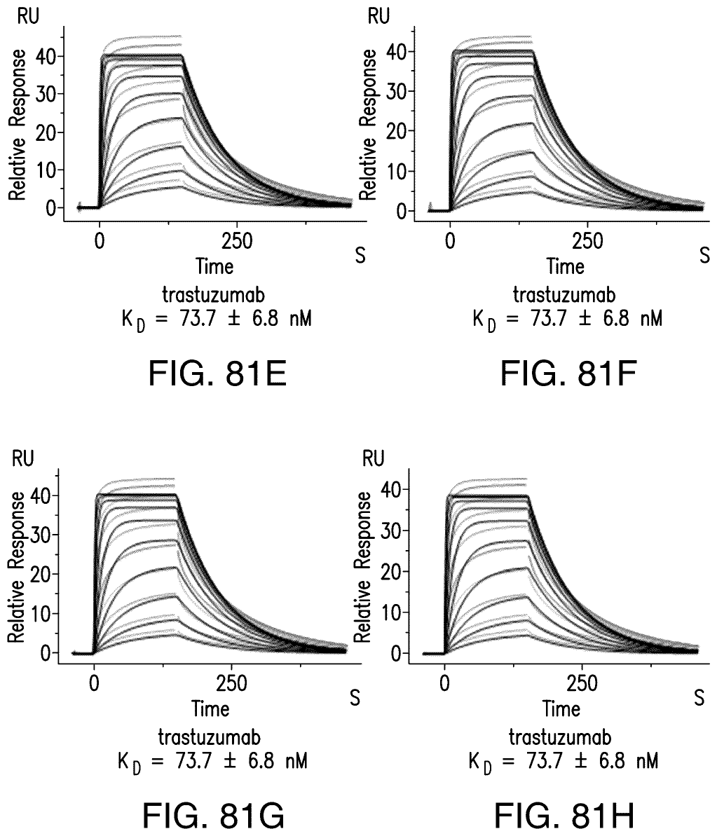

Binding of AB1424/AB16121F3′ TriNKET to cynomolgus CD16 was measured and is shown in FIG. 81A-FIG. 81H. The resulting affinity values determined from the sensorgrams are summarized in Table 48.

TABLE 48
Affinity values of AB1424/AB1612 F3' TriNKET and
trastuzumab for cynomolgus CD16.

| | kₐ (1/Ms) | k_{d} (1/s) | K_{D} (nM) |
|---|---|---|---|
| AB1424/ | 7.8 × 10⁴ | 2.0 × 10⁻² | 260.9 |
| AB1612 F3' | | | |
| TriNKET | | | |
| AB1424/' | 7.8 × 10⁴ | 2.1 × 10⁻² | 268.8 |
| AB1612 F3 | | | |
| TriNKET | | | |
| AB1424/ | 7.2 × 10⁴ | 2.0 × 10⁻² | 282.6 |
| AB1612 F3' | | | |
| TriNKET | | | |
| AB1424/ | 7.8 × 10⁴ | 2.0 × 10⁻² | 260.4 |
| AB1612 F3' | | | |
| TriNKET | | | |
| Average ± SD | (7.6 ± 0.4) × 10⁴ | (2.1 ± 0.0) × 10⁻² | 270.8 ± 11.0 |
| trastuzumab | 1.9 × 10⁵ | 1.2 × 10⁻² | 66.1 |
| trastuzumab | 1.6 × 10⁵ | 1.2 × 10⁻² | 76.2 |
| trastuzumab | 1.6 × 10⁵ | 1.2 × 10⁻² | 78.9 |
| trastuzumab | 1.6 × 10⁵ | 1.3 × 10⁻² | 79.0 |
| Average ± SD | (1.7 ± 0.2) × 10⁵ | (1.2 ± 0.0) × 10⁻² | 73.7 ± 6.8 |

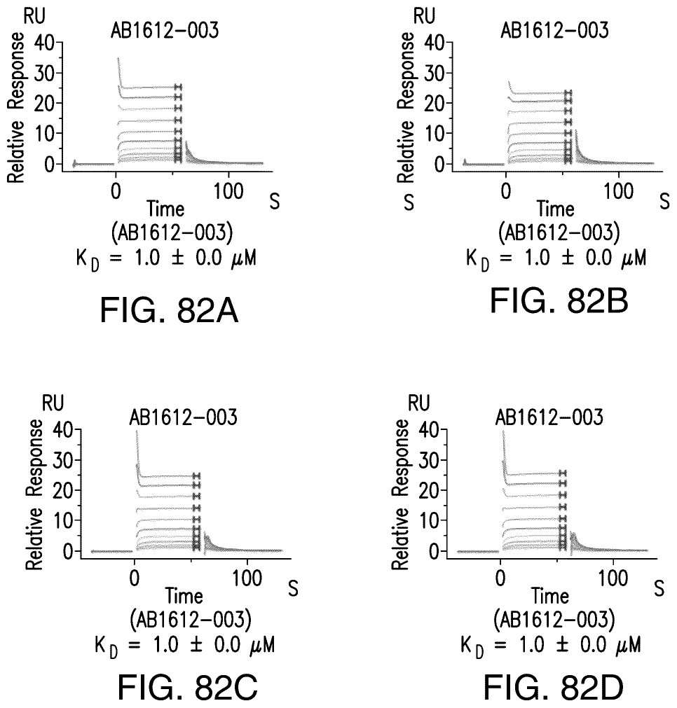

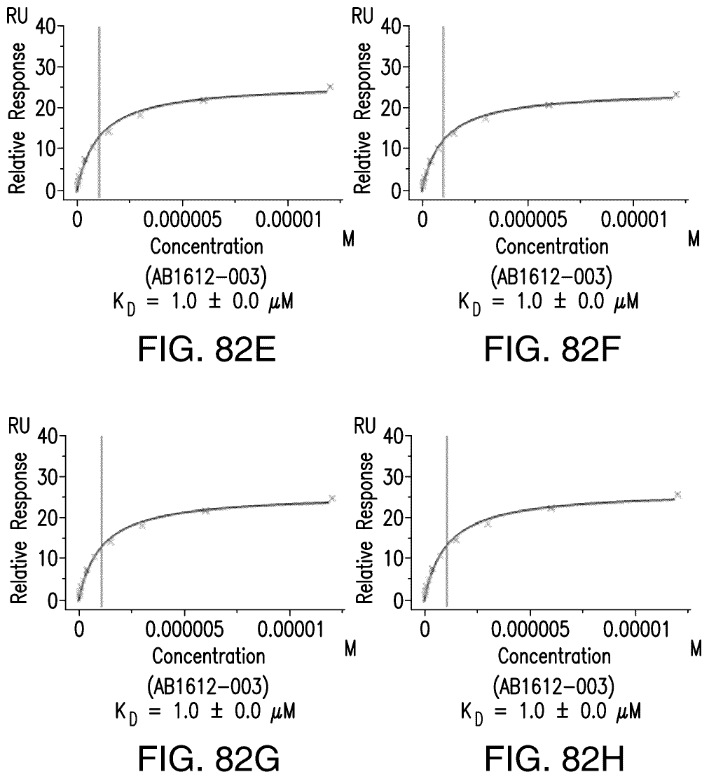

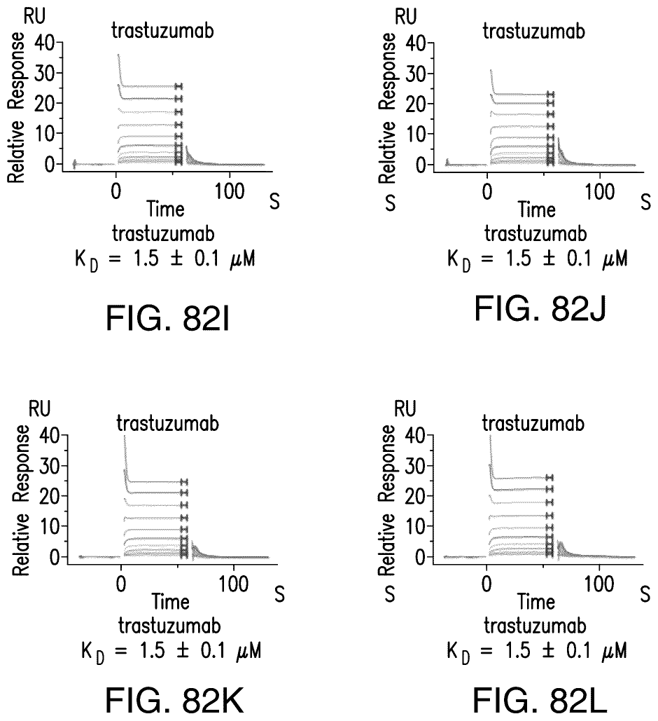

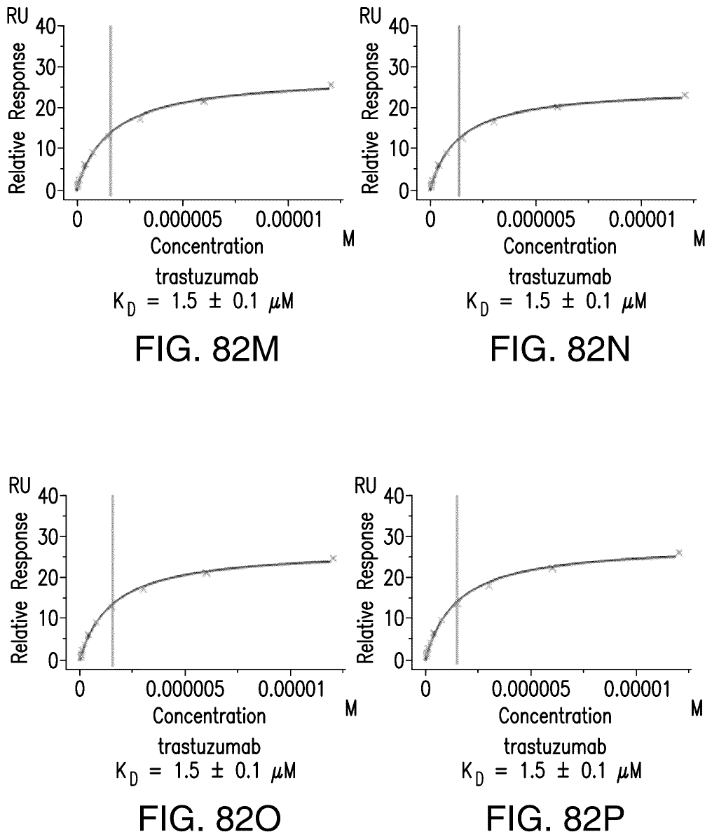

Binding of AB1424/AB1612 F3′ TriNKET to human FcRn was measured at pH 6.0 and is shown in FIG. 82A-FIG. 82P. The resulting affinity values determined from the sensorgrams are summarized in Table 49.

TABLE 49
Affinity values of AB1424/AB1612 F3' TriNKET
and trastuzumab for human FcRn at pH 6.0.

| | Rep- | Rep- | Rep- | Rep- | K_{D} (μM) |
|---|---|---|---|---|---|
| | licate | licate | licate | licate | Average ± |
| Sample | K_{D} (μM) | K_{D} (μM) | K_{D} (μM) | K_{D} (μM) | SD |
| AB1424/AB16 | 1.0 | 1.0 | 1.1 | 1.1 | 1.0 ± 0.0 |
| 12 F3' | | | | | |
| TriNKET | | | | | |
| trastuzumab | 1.6 | 1.4 | 1.5 | 1.5 | 1.5 ± 0.1 |

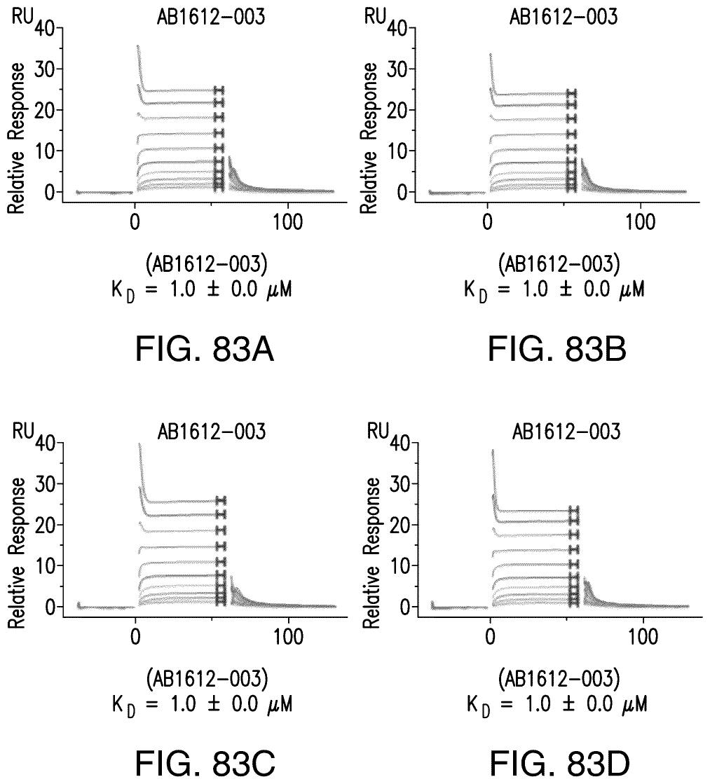

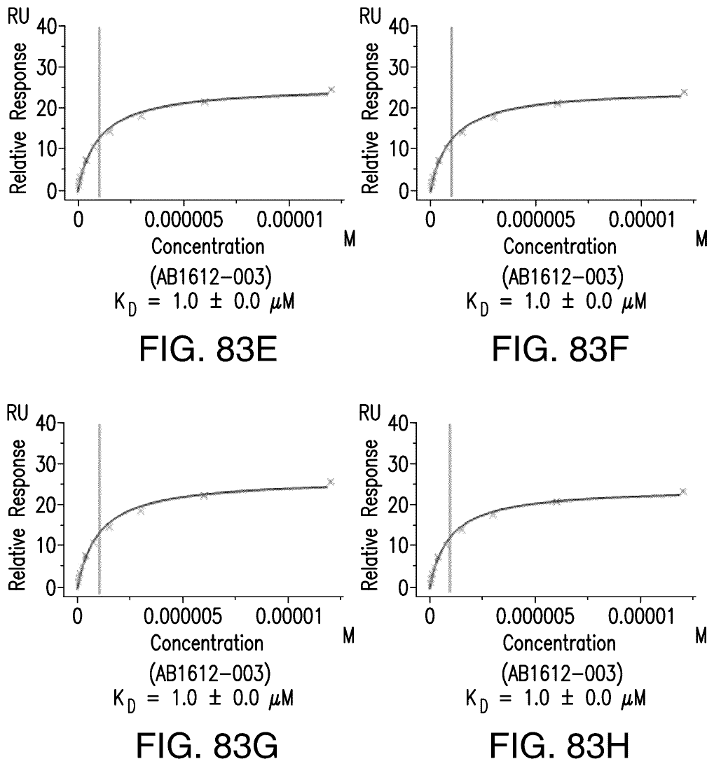

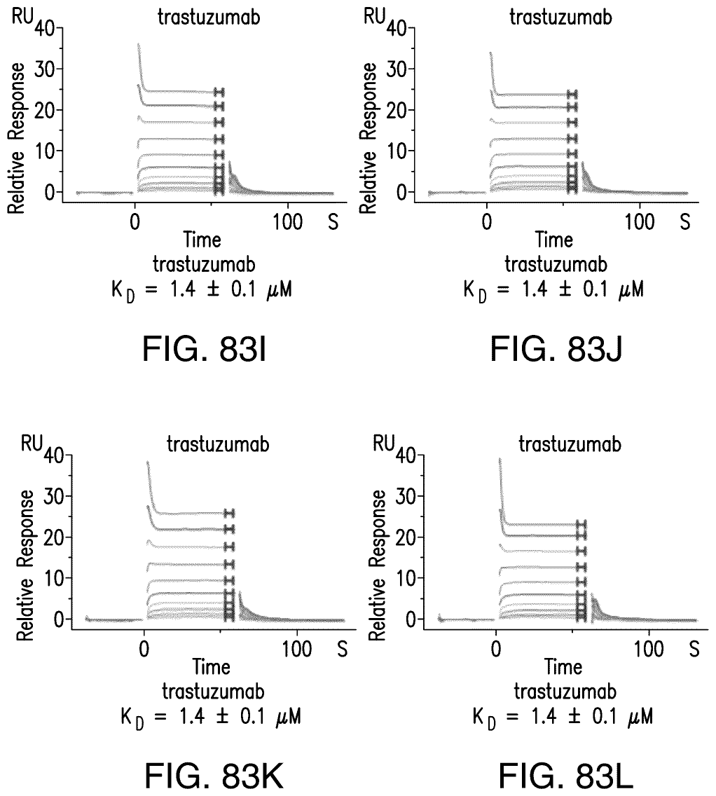

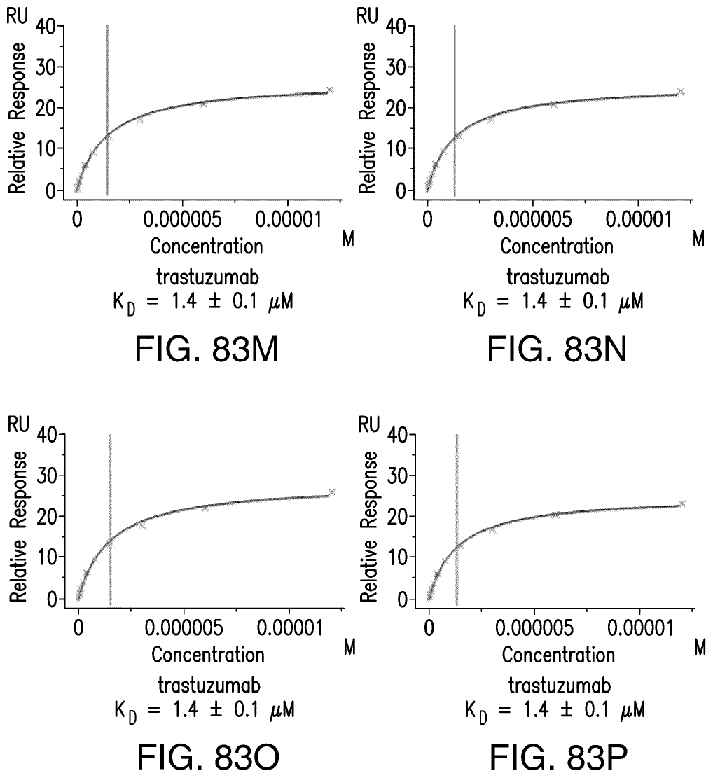

Binding of AB1424/AB16121F3′ TriNIKET to cynomolgus FcRn was measured and is shown in FIG. 83A-FIG. 83P. The resulting affinity values determined from the sensorgrams are summarized in Table 50.

TABLE 50
Affinity values of AB1424/AB1612 F3' TriNKET and
trastuzumab for cynomolgus FcRn at pH 6.0.

| | Rep- | Rep- | Rep- | Rep- | K_{D} (μM) |
|---|---|---|---|---|---|
| | licate | licate | licate | licate | Average ± |
| Sample | K_{D} (μM) | K_{D} (μM) | K_{D} (μM) | K_{D} (μM) | SD |
| AB1424/AB16 | 1.0 | 1.0 | 1.0 | 1.0 | 1.0 ± 0.0 |
| 12 F3' | | | | | |
| TriNKET | | | | | |
| trastuzumab | 1.5 | 1.3 | 1.5 | 1.3 | 1.4 ± 0.1 |

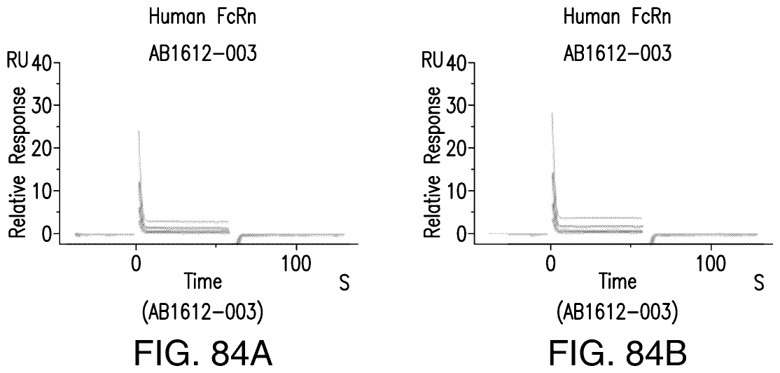

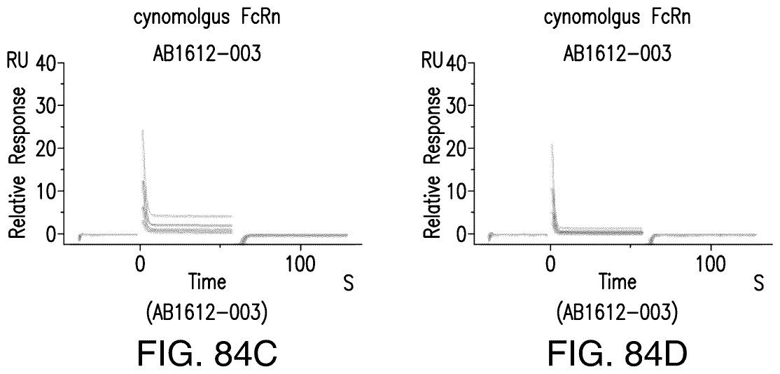

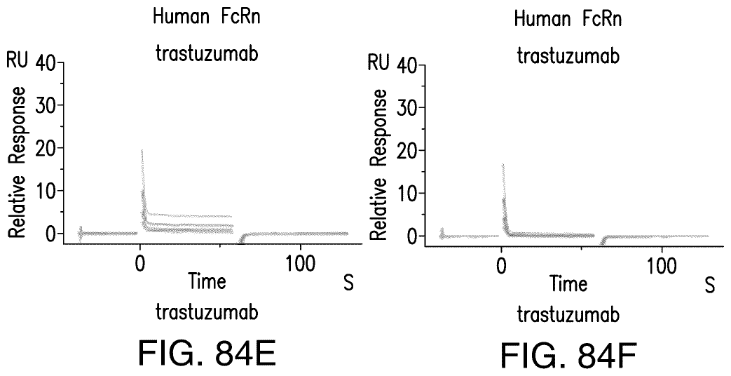

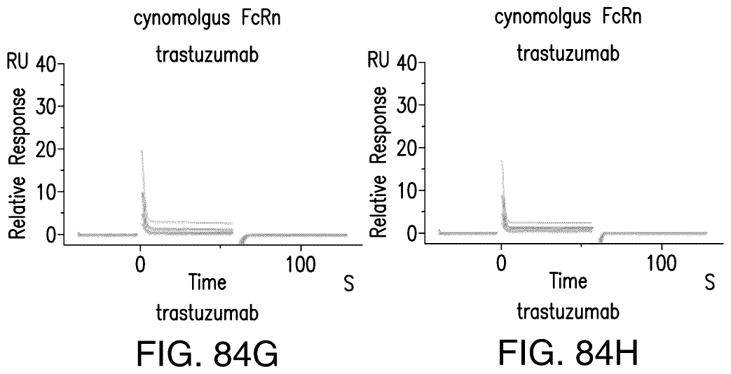

The lack of quantifiable binding of AB1424/AB1612 F3′ TriNKET and trastuzumab to human and cynomolgus FcRn at pH 7.4 was demonstrated and is shown in FIG. 84A-FIG. 84H. The pH 7.4 binding was run on the same Biacore chip surface prior to the pH 6.0 FcRn binding experiment.

AB1424/AB1612 F3′ TriNKET and an IgG1 isotype control trastuzumab did not demonstrate physiologically meaningful differences (less than 3-fold different) and are comparable in their binding to human and cynomolgus CD64 (FcγRI) and CD16 (FcγRIII) recombinant receptors tested (Table 50). AB1424/AB1612 F3′ TriNKET and trastuzumab are similar (less than 1.2-fold different) in their binding to human CD32 (FcγRII) receptors (Table 51).

TABLE 51
Summary of FcγRs affinities for AB1424/AB1612 F3' TriNKET and trastuzumab.

| | Human | Cyno | Human CD32a | | Human CD16a | | Human | Human | Cyno |
|---|---|---|---|---|---|---|---|---|---|
| | CD64 | CD64 | K_{D}, μM | | K_{D}, nM | | CD32b | CD16b | CD16 |
| Analyte | K_{D}, nM | K_{D}, nM | H131 | R131 | V158 | F158 | K_{D}, μM | K_{D}, μM | K_{D}, nM |
| AB1424/ | 3.2 ± 0.2 | 2.1 ± 0.6 | 1.0 ± 0.1 | 1.4 ± 0.1 | 129.3 ± 5.2 | 1346.2 ± 72.6 | 5.9 ± 0.3 | 6.9 ± 0.7 | 270.8 ± 11.0 |
| AB1612 | | | | | | | | | |
| F3' | | | | | | | | | |
| TriNKET | | | | | | | | | |
| trastuzumab | 2.7 ± 0.9 | 0.8 ± 0.1 | 1.3 ± 0.1 | 1.7 ± 0.2 | 50.3 ± 3.9 | 448.1 ± 20.1 | 7.2 ± 0.5 | 3.8 ± 0.5 | 73.7 ± 6.8 |

Additionally, AB1424/AB1612 F3′ TriNKET is similar to trastuzumab in its affinity for human and cynomolgus FcRn at pH 6.0. AB1424/AB1612 F3′ TriNKET, and trastuzumab did not demonstrate any detectable binding at the concentrations tested at pH 7.4 (Table 52).

TABLE 52
Summary of AB1424/AB1612 F3′ TriNKET and
trastuzumab binding human and cynomolgus

| | Human | Human | Cyno | Cyno |
|---|---|---|---|---|
| | FcRn, | FcRn, | FcRn, | FcRn, |
| | pH 6.0 | pH 7.4 | pH 6.0 | pH 7.4 |
| Analyte | K_{D}, μM | K_{D}, μM | K_{D}, μM | K_{D}, μM |
| AB1424/ | 1.0 ± 0.0 | No | 1.0 ± 0.0 | No |
| AB1612 F3′ | | quantifiable | | quantifiable |
| TriNKET | | binding | | binding |
| trastuzumab | 1.5 ± 0.1 | No | 1.4 ± 0.1 | No |
| | | quantifiable | | quantifiable |
| | | binding | | binding |

### Example 6—Potency Analysis AB1424/AB1612 F3 TriNKET

KHYG-1 CD16V mediated cytotoxicity assays were performed as described in Example 4.

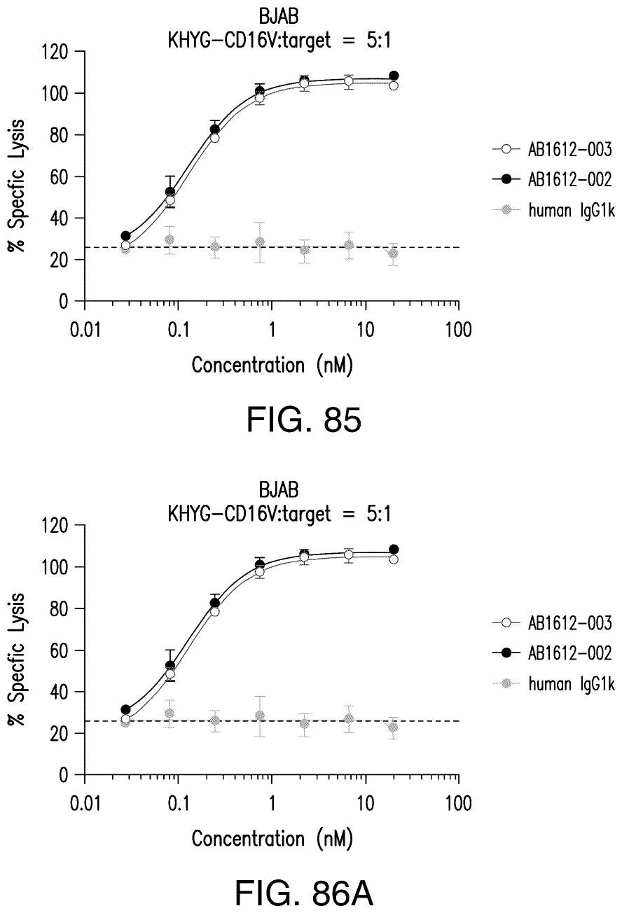

Two different production lots of AB1424/AB1612 F3′ TriNKET were tested for potency as shown in FIG. 85. AB1424/AB1612 F3′ TriNKET expressed in expiCHO cells (AB1612-002) was compared to a lot of AB1424/AB1612 F3′ TriNKET expressed in CHO-M cells (AB1612-003). Both molecules demonstrated comparable potency and maximum lysis of BJAB target cells (shown in Table 53). These results demonstrate the consistency of AB1424/AB1612 F3′ TriNKET potency across two different production lots and two different expression systems. In addition, these data showed that the cytotoxicity assay using KHYG-1-CD16V and BJAB cells is robust and could serve as a lot release bioassay.

TABLE 53
KHYG-1-CD16V EC₅₀ values

| Molecule | EC₅₀ (nM) | % Max lysis |
|---|---|---|
| AB1612-002 | 0.13 | 108 |
| AB1612-003 | 0.13 | 105 |

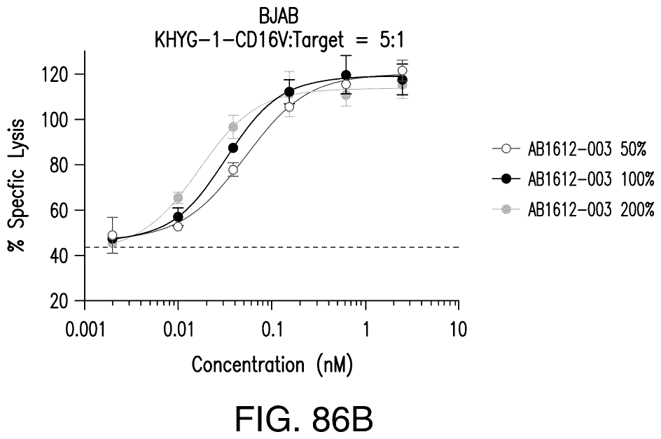

Sensitivity of KHYG-1-CD16V+ BJAB assay to detect changes in potency of AB1424/AB1612 F3′ TriNKET was determined. FIG. 86B shows dose response curves using 100% AB1424/AB1612 F3′ TriNKET as reference standard (EC50=0.03 nM) compared to the molecule at 200% nominal drug concentration (NDC) and 50% NDC. The relative potencies of 200% AB1424/AB1612 F3′ TriNKET and 50% AB1424/AB1612 F3′ TriNKET were calculated by normalizing EC₅₀ values to the EC₅₀ of 100% AB1424/AB1612 F3′ TriNKET. Using 100% AB1424/AB1612 F3′ TriNKET as a reference, the higher concentration (200% AB1424/AB1612 F3′ TriNKET) showed a relative potency of 200%, and lower concentration (50% AB1424/AB1612 F3′ TriNKET) showed a relative potency of 65%. The relative potencies of 200%, 100%, and 50% NDCs of AB1424/AB1612 F3′ TriNKET using KHYG-1-CD16V effector cells and BJAB target cells suggest that the shift in the EC50 values observed in this cell-based potency assay are nearly linear in the range of 50%-200% nominal drug concentration (Table 54).

TABLE 54
KHYG-1 CD16V EC₅₀ values

| Molecule | EC₅₀ (nM) | % Max lysis |
|---|---|---|
| 50% | 0.05 | 120 |
| AB1424/AB1612 | | |
| F3′ TriNKET | | |
| 100% | 0.03 | 119 |
| AB1424/AB1612 | | |
| F3′ TriNKET | | |
| 200% | 0.02 | 114 |
| AB1424/AB1612 | | |
| F3′ TriNKET | | |

### Example 7—High Concentration of AB1424/AB1612 F3 TriNKET Feasibility and Stability Analysis

### PEG Precipitation

A PEG precipitation study was conducted to determine stability of AB1424/AB1612 F3′ TriNKET. Briefly, colloidal stability was evaluated in 10 mM acetate pH 5.0 and 20 mM histidine pH 6.0. For each buffer, a 40% w/v stock solution of PEG-6000 was made, and pH was adjusted to 5.0 for acetate containing solution and pH 6.0 for histidine containing solution. A PEG-6000 titration curve was generated from the PEG stock, buffer stock, and protein stock solutions (at 36.9 mg/mL or 34.4 mg/mL in PBS), and no buffer exchange was necessary due to high dilution factor to 1 mg/mL). The PEG titration curve covered concentrations from 0 to 30% w/v PEG-6000, and each point was prepared in triplicate for adalimumab control or AB1424/AB1612 F3′ TriNKET in each buffer. After mixing solutions, samples were incubated at 5° C. overnight and centrifuged at 15,000 rpm for 10 minutes (in a pre-cooled, 5° C. centrifuge) to remove precipitated protein. The supernatant was then removed, and concentration was read by absorbance at 280 nm. The concentrations were then plotted against PEG concentration to determine midpoints (Cₘ); Cₘ>20% PEG-6000 was considered good colloidal stability.

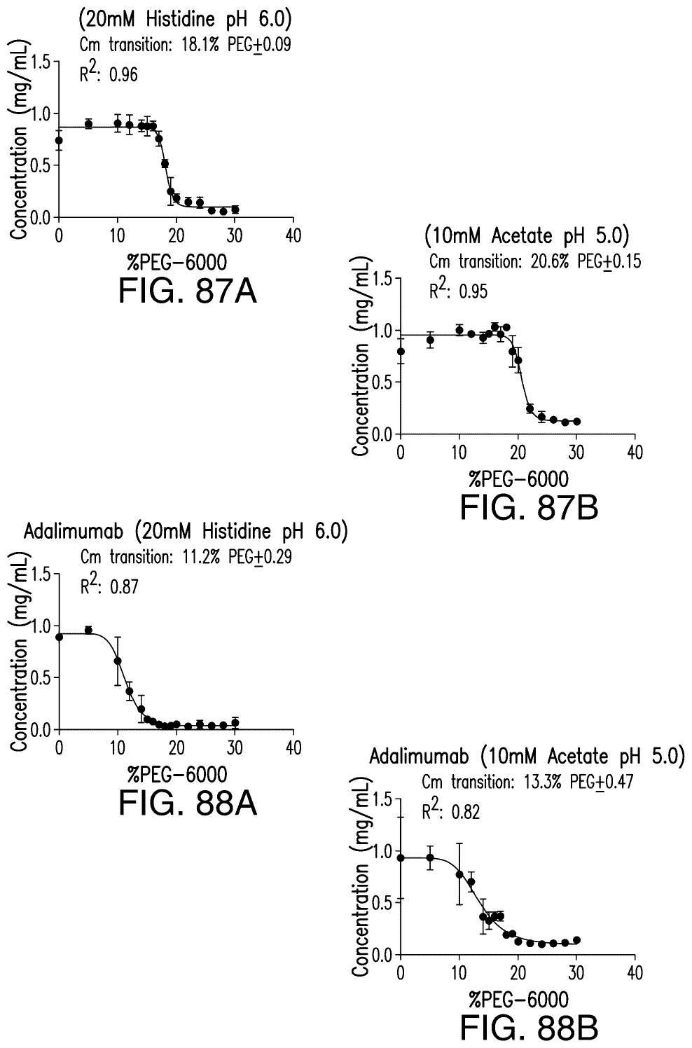

Colloidal stability of AB1424/AB1612 F3′TriNKET was studied in two buffers (20 mM histidine, pH 6.0 and 10 mM acetate, pH 5.0) using a PEG precipitation assay. Adalimumab was used as a benchmarking reference of a well-behaved commercial biotherapeutic antibody. In both buffers, AB1424/AB1612 F3′ TriNKET showed higher colloidal stability than adalimumab, lending confidence to its ability to be concentrated to high protein concentrations (FIG. 87A and FIG. 87B, FIG. 88A and FIG. 88B, Table 55). In histidine, AB1424/AB1612 F3′ TriNKET showed Cₘ 18.1±0.09, while in acetate the Cₘ was 20.6±0.15, satisfying high colloidal stability criteria.

TABLE 55
Summary of PEG Precipitation Cm Summary.

| | 10 mM Acetate, pH 5 | | 20 mM Histidine, pH 6 | |
|---|---|---|---|---|
| | | AB1424/ | | AB1424/A |
| | | AB1612 | | B1612 |
| Cₘ | | F3′ | | F3′ |
| (% PEG) | Adalimumab | TriNKET | Adalimumab | TriNKET |
| Concentration | 13.3 ± 0.5 | 20.6 ± 0.2 | 11.2 ± 0.3 | 18.1 ± 0.1 |

### Dynamic Light Scattering (DLS)

The self-interacting propensity of AB1424/AB1612 F3′ TriNKET was explored by DLS in three different buffers with range of pH from 5.0 to 7.0 (20 mM acetate, pH 5.0; 20 mM histidine, pH 6.0; or 20 mM phosphate, pH 7.0). For DLS, briefly, k_{D} was determined using the Nanotemper Prometheus Panta, run in high sensitivity DLS mode. In short, samples were prepared in buffer and 10 μL was loaded into three individual capillaries for analysis per concentration. The results were fit in the Panta Analysis software, and k_{D} values were calculated for each buffer individually. Adalimumab was used as an example of a well-behaved commercial biologic known to be capable of formulation and administration at high concentration.

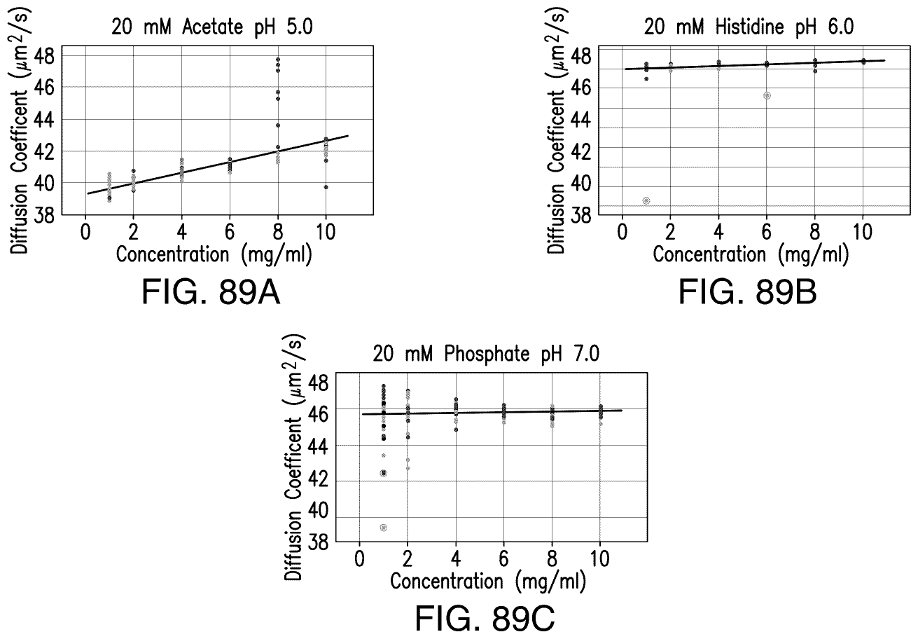

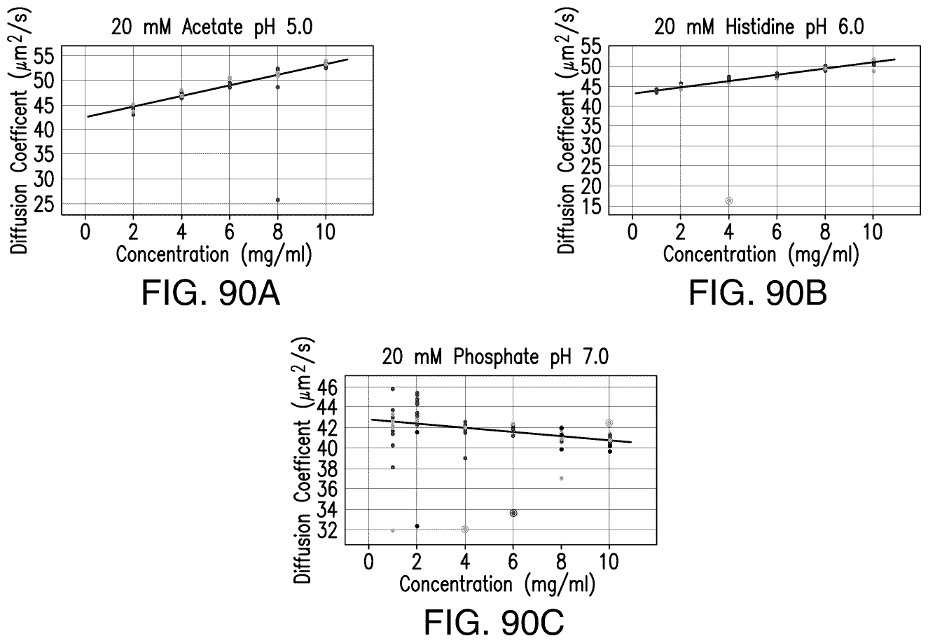

Like the findings from the PEG precipitation, when comparing k_{D} (by DLS) values between buffers for both molecules, acetate and histidine exhibited strong positive values, whereas phosphate exhibited negative or slightly positive values as shown in FIG. 89A, FIG. 89B, FIG. 90A, FIG. 90B, and Table 56. In both acetate and histidine, AB1424/AB1612 F3′ TriNKET exhibited equivalent or better k_{D} compared to adalimumab, as judged by the magnitude of the positive value. These data corroborate the findings from PEG precipitation, with AB1424/AB1612 F3′ TriNKET outperforming adalimumab in both acetate and histidine buffers. Both PEG precipitation and DLS strongly indicated that AB1424/AB1612 F3′TriNKET has high conformational and colloidal stability and are amendable to high concentration formulation.

TABLE 56
Self-Interaction (k_{D}) Summary.

| | 20 mM | 20 mM | 20 mM |
|---|---|---|---|
| | Acetate, | Histidine, | Phosphate, |
| Test article | pH 5 | pH 6 | pH 7 |
| Adalimumab | +8.5 | +5.0 | +1.1 |
| AB1424/AB1612 | +25.4 | +18.3 | −4.9 |
| F3′TriNKET | | | |

### Concentration Feasibility Study

The concentration feasibility study, performed at small scale, indicated AB1424/AB1612 F3′ TriNKET could be concentrated to about 150 mg/mL. Overall, the yield was 88.5% based on starting/ending quantity of protein as shown in Table 57. The samples were of high purity and matched expected molecular weight as shown by SEC-MALS in Table 58.

TABLE 57
Summary of Feasibility Material Concentration.

| | | Start | | Final | Final | | |
|---|---|---|---|---|---|---|---|
| Spin | Start Volume | Concentration | Protein | Volume | Concentration | Protein | % |
| # | (mL) | (mg/mL) | (mg) | (mL) ^{a} | (mg/mL) | (mg) | Recovery |
| 1 | 8.0 | 12.8 | 102.5 | 3.3 | 31.3 | 101.7 | 99.2 |
| 2 | 3.1 | 31.3 | 95.4 | 1.5 | 66.2 | 98.6 | 103.4 |
| 3 | 1.4 | 66.2 | 92.0 | 0.9 | 101.4 | 95.4 | 103.6 |
| 4 | 0.8 | 101.4 | 85.2 | 0.7 | 126.1 | 87.0 | 102.1 |
| 5 | 0.6 | 126.1 | 80.7 | 0.6 | 146.3 | 86.3 | 107.0 |

| | | |
|---|---|---|
| Final recovery (concentrate, retains, wash) | 90.7 | 88.5 |

^{a} Final volume for intermediate spins was determined by manual pipetting permeate and subtracting volume from starting volume. Final % Recovery was determined by measuring volume of retentate.

TABLE 58
Summary of Feasibility Material SEC-MALS Analysis.

| | % | % | Monomer Mw |
|---|---|---|---|
| Sample | HMWS | Monomer | (kDa ± uncertainty) ^{a} |
| Starting Material | 0.2 | 99.8 | 110.3 ± 0.9% |
| Spin 1 | 0.2 | 99.8 | 115.2 ± 0.8% |
| Spin 2 | 0.3 | 99.7 | 115.9 ± 0.4% |
| Spin 3 | 0.3 | 99.7 | 116.9 ± 0.7% |
| Spin 4 | 0.3 | 99.7 | 117.7 ± 0.8% |
| Spin 5/Final Material | 0.3 | 99.7 | 117.9 ± 0.7% |

^{a} Theoretical Mw based on sequence is ~125 kDa

### Bulk Material Concentration

About 350 mg of ABL424/AB1612 F3′ TriNKET was concentrated to about 140 mg/mL for use in thermal stability evaluation and viscosity determination. The large batch of high concentration material, used for accelerated stability and viscosity, was generated separate from the feasibility batch. The material was generated from buffer exchanged AB1424/AB1612 F3′ TriNIKET in HST and like the feasibility study, yielded high quantity of material of high monomer content by SEC, and is summarized in Table 59.

TABLE 59
Summary of Bulk Material Concentration.

| | Start | Start | | Final | Final | | |
|---|---|---|---|---|---|---|---|
| Spin | Volume | Concentration | Protein | Volume | Concentration | Protein | % |
| # | (mL) | (mg/mL) | (mg) | (mL) ^{a} | (mg/mL) | (mg) | Recovery ^{b} |
| 1 | 25.5 | 13.5 | 344.3 | 14.5 | 23.50 | 339.6 | 98.6 |
| 2 | 14.5 | 23.5 | 339.6 | 10.1 | 33.90 | 340.7 | 100.3 |
| 3 | 10.1 | 33.9 | 340.7 | 6.8 | 49.50 | 336.6 | 98.8 |
| 4 | 6.8 | 49.5 | 336.6 | 3.7 | 110.50 | 408.9 | 121.5 |
| 5 | 3.7 | 110.5 | 408.9 | 3.2 | 136.40 | 429.7 | 105.1 |
| 6 | 3.2 | 136.4 | 429.7 | 1.9 | 154.1 | 288.2 | 62.5 |

| | | |
|---|---|---|
| Final recovery (concentrate, retains, wash) | 303.5 | 89.4 |

^{a} Final volume for intermediate spins was determined by manual pipetting permeate and subtracting volume from starting volume. Final % Yield was determined by measuring volume of retentate.
^{b} Note: only final % Recovery should be considered when assessing recovery of the concentration process.

TABLE 60
Summary of Bulk Material Concentration SEC Analysis

| | Concentration | % | % |
|---|---|---|---|
| Sample | (mg/mL) | HMWs | Monomer |
| Starting Material | 13.5 | 0.3 | 99.7 |
| Spin 1 | 23.5 | 0.5 | 99.5 |
| Spin 2 | 33.9 | 0.3 | 99.7 |
| Spin 3 | 49.5 | 0.3 | 99.7 |
| Spin 4 | 110.5 | 0.3 | 99.7 |
| Spin 5 | 136.4 | 0.3 | 99.7 |
| Spin 6 | 154.1 | 0.4 | 99.6 |
| Final Material | 154.1 | 0.3 | 99.7 |

### Viscosity Determination

Viscosity of the formulation was determined across a concentration range of 0 to 140 mg/mL of AB1424/AB1612 F3′ TriNKET, formulated in HST buffer at 25° C. Concentrations were as follows, 0, 5, 15, 25, 75, 100, 120 and 140 mg/mL. Samples were analyzed by RheoSense (San Ramon, CA) using the VROC® initium high throughput viscometer, equipped with a B05 flow channel (depth=50 μm, Pₘₐₓ=42 kPa). A NIST traceable Newtonian standard oil (Cannon N10 Lot 19201, 15.84 cP at 25° C.) was tested to confirm consistent performance of the flow channel and instrument prior to analyzing samples. Buffer and concentrations 5 through 75 mg/mL were measured at maximum shear rate of 22,040 sec⁻¹. Shear rate sweeps were performed on the three highest concentrations (100, 120 and 140 mg/mL).

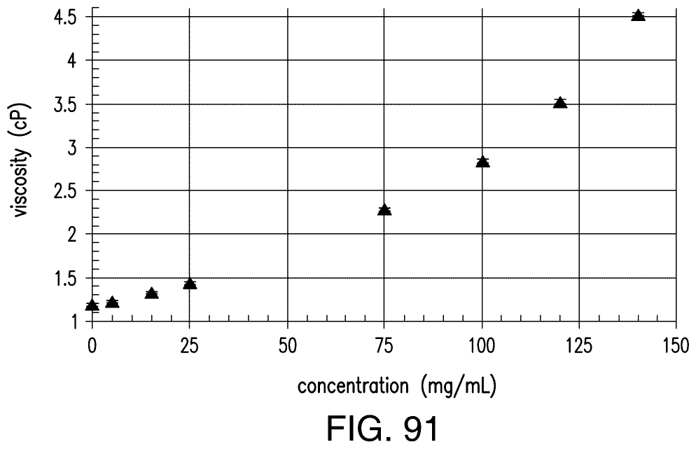

The viscosity of AB1424/AB1612 F3′ TriNKET formulated at a concentration range from 5 to 140 mg/mL in HST buffer was within the acceptable range (<20 cP) as determined by RheoSense. The viscosity at the highest concentration (140 mg/mL) was only 4.5 cP, well within the acceptable viscosity range of <20 cP for autoinjector solutions. Results are shown in FIG. 91 and Table 61.

TABLE 61
Summary of Viscosity Analysis at 25° C.

| | | Con- | Average | | | |
|---|---|---|---|---|---|---|
| | | centration | Viscosity | STDEV | % | |
| Sample | Buffer | (mg/mL) | (cP) | (cP) | RSD | N |
| AB1424/ | HST | 0 | 1.186 | 0.005 | 0.40 | 11 |
| AB1612 | | | | | | |
| F3' | | | | | | |
| TriNKET | | | | | | |
| AB1424/ | HST | 5 | 1.225 | 0.002 | 0.20 | 13 |
| AB1612 | | | | | | |
| F3' | | | | | | |
| TriNKET | | | | | | |
| AB1424/ | HST | 15 | 1.326 | 0.004 | 0.27 | 13 |
| AB1612 | | | | | | |
| F3' | | | | | | |
| TriNKET | | | | | | |
| AB1424/ | HST | 25 | 1.437 | 0.004 | 0.28 | 12 |
| AB1612 | | | | | | |
| F3' | | | | | | |
| TriNKET | | | | | | |
| AB1424/ | HST | 75 | 2.291 | 0.004 | 0.16 | 12 |
| AB1612 | | | | | | |
| F3' | | | | | | |
| TriNKET | | | | | | |
| AB1424/ | HST | 100 | 2.844 | 0.007 | 0.26 | 19 |
| AB1612 | | | | | | |
| F3' | | | | | | |
| TriNKET | | | | | | |
| AB1424/ | HST | 120 | 3.524 | 0.008 | 0.22 | 18 |
| AB1612 | | | | | | |
| F3' | | | | | | |
| TriNKET | | | | | | |
| AB1424/ | HST | 140 | 4.528 | 0.006 | 0.14 | 20 |
| AB1612 | | | | | | |
| F3' | | | | | | |
| TriNKET | | | | | | |

### High Concentration Accelerated (40° C.) Stability in HST, pH 6.0

To explore if AB11424/AB16121F3′ TriNIKET is stable at high concentration, an accelerated stability study was performed by incubating AB1424/AB16121F3′ TriNIKET at 40° C. over 4 weeks in HST formulation and assessing the structural and functional stability of the protein by A280, turbidity, opalescence, SEC, CE-SDS, cIEF, BAFFR+ cell binding, SPR, and potency. After 4 weeks, the concentration of AB1424/AB16121F3′ TriNIKET increased slightly from 135 mg/mL to 160 mg/mL consistent with some evaporation at elevated temperature. Turbidity and Opalescence did not change over time (Table 62).

TABLE 62
Summary of AB1424/AB1612 F3′ TriNKET UV-VIS
Results After Incubation at 40° C. in HST, pH 6.0.

| | Concen- | | |
|---|---|---|---|
| | tration | Turbidity | Opalescence |
| Test Article | (mg/mL) | (A350 nm) | (A500 nm) |
| AB1424/AB1612 F3′ TriNKET, | 135.4 | 0.3 | <0.1 |
| HST, pH 6.0, control | | | |
| AB1424/AB1612 F3′ TriNKET, | 147.7 | 0.3 | <0.1 |
| HST, pH 6.0, 40° C., 1 week | | | |
| AB1424/AB1612 F3′ TriNKET, | 142.0 | 0.3 | <0.1 |
| HST, pH 6.0, 40° C., 2 weeks | | | |
| AB1424/AB1612 F3′ TriNKET, | 146.9 | 0.3 | <0.1 |
| HST, pH 6.0, 40° C., 3 weeks | | | |
| AB1424/AB1612 F3′ TriNKET, | 160.9 | 0.4 | <0.1 |
| HST, pH 6.0, 40° C., 4 weeks | | | |

### Size-Exclusion Chromatography (SEC)

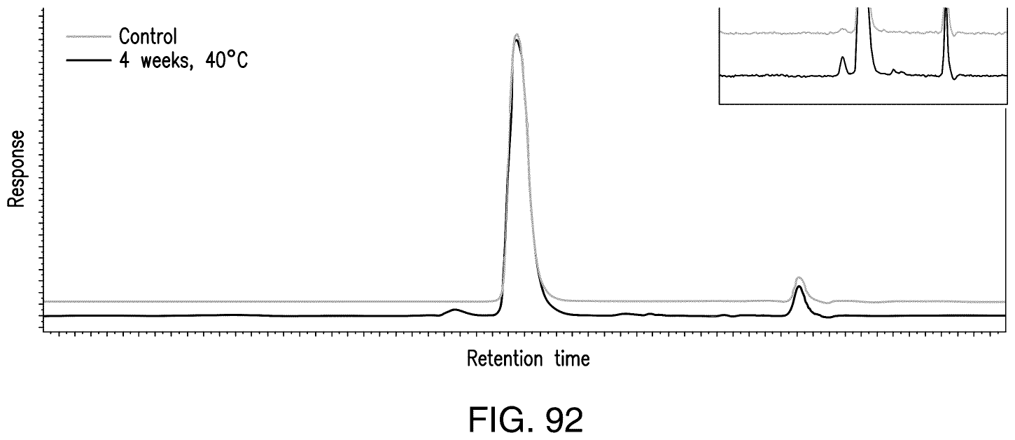

SEC was performed as described in Example 4. High concentration AB1424/AB1612 F3′ TriNKET demonstrated high stability after 4 weeks of incubation in HST, pH 6.0, at 40° C. HMWS increased minimally from 1.2% to 2.0%, LMWS increased from 1.1% to 1.4% and the Monomer decreased from 99.4% to 97.0% after 4 weeks, indicating that high concentration has no meaningful impact on aggregation during stress evaluation (FIG. 92 and Table 63).

TABLE 63
Summary of AB1424/AB1612 F3′ TriNKET SEC Results
after incubation at 40° C. in HST, pH 6.0.

| | Monomer | HMWS | LMWS |
|---|---|---|---|
| Test Article | (%) | (%) | (%) |
| AB1424/AB1612 F3′ TriNKET, | 99.4 | 0.6 | 0.0 |
| HST, pH 6.0, control | | | |
| AB1424/AB1612 F3′ TriNKET, | 98.6 | 1.4 | 0.0 |
| HST, pH 6.0, 40° C., 1 week | | | |
| AB1424/AB1612 F3′ TriNKET, | 98.5 | 1.5 | 0.0 |
| HST, pH 6.0, 40° C., 2 weeks | | | |
| AB1424/AB1612 F3′ TriNKET, | 96.9 | 2.2 | 0.9 |
| HST, pH 6.0, 40° C., 3 weeks | | | |
| AB1424/AB1612 F3′ TriNKET, | 97.0 | 2.0 | 1.0 |
| HST, pH 6.0, 40° C., 4 weeks | | | |

### CE-SDS (Reduced)

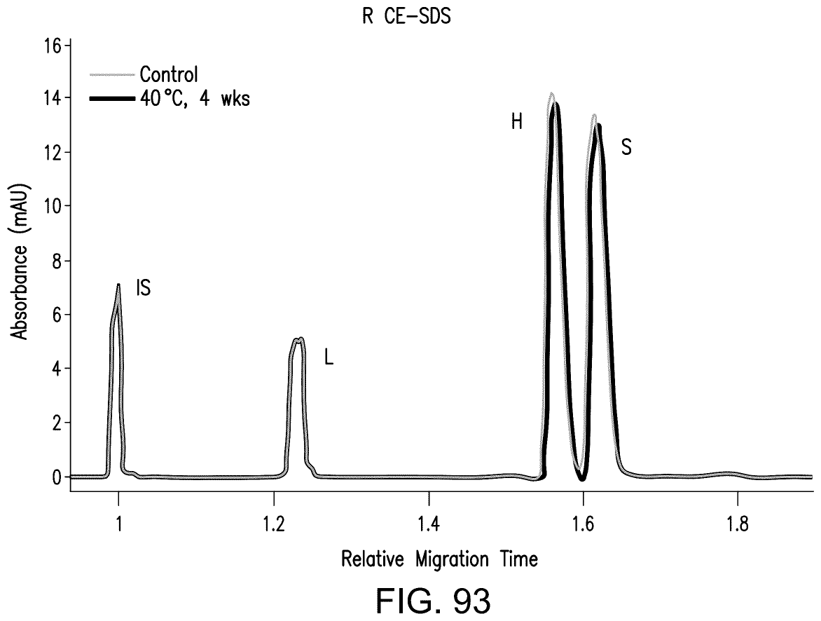

Purity assessed by reduced CE-SDS showed a 0.1% loss in purity over the 4-week incubation at 40° C. (FIG. 93 and Table 64). Under reduced conditions, the three expected chains (LC, HC, and scFv-Fc chain) were observed.

TABLE 64
Summary of AB1424/AB1612 F3′ TriNKET R CE-SDS
Purity After 4 Weeks at 40° C. in HST, pH 6.0.

| Test Article | R CE-SDS Purity (%) |
|---|---|
| AB1424/AB1612 F3′ TriNKET, | 99.7 |
| HST, pH 6.0, control | |
| AB1424/AB1612 F3′ TriNKET, | 99.8 |
| HST, pH 6.0, 40° C., 1 week | |
| AB1424/AB1612 F3′ TriNKET, | 99.7 |
| HST, pH 6.0, 40° C., 2 weeks | |
| AB1424/AB1612 F3′ TriNKET, | 99.5 |
| HST, pH 6.0, 40° C., 3 weeks | |
| AB1424/AB1612 F3′ TriNKET, | 99.6 |
| HST, pH 6.0, 40° C., 4 weeks | |

Capillary Isoelectric Focusing (cIEF)

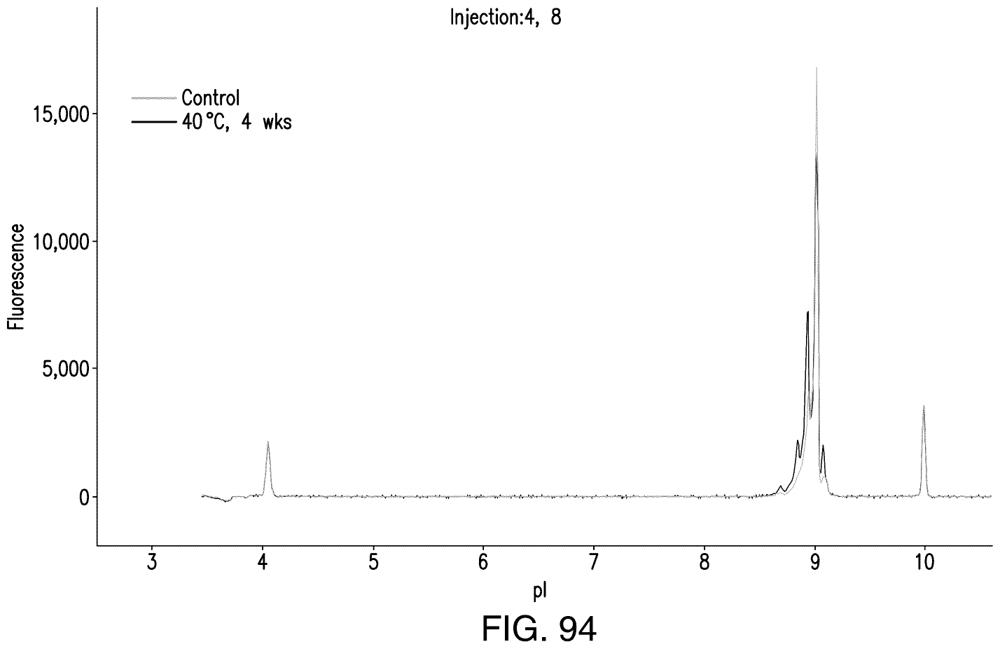

cIEF was performed as described in Example 4. The charge profile as determined by cIEF demonstrated an acidic shift from 55.7% main peak in the control to 44.3% main peak after 4 weeks at 40° C. in HST (FIG. 94 and Table 65).

TABLE 65
Summary of AB1424/AB1612 F3′ TriNKET iCIEF
Results After Incubation at 40° C. in HST, pH 6.0.

| Test Article | Acidic (%) | Main (%) | Basic (%) |
|---|---|---|---|
| AB1424/AB1612 F3′ TriNKET, | 39.7 | 55.7 | 4.8 |
| HST, pH 6.0, control | | | |
| AB1424/AB1612 F3′ TriNKET, | 40.2 | 53.7 | 6.1 |
| HST, pH 6.0, 40° C., 1 week | | | |
| AB1424/AB1612 F3′ TriNKET, | 42.9 | 50.5 | 6.5 |
| HST, pH 6.0, 40° C., 2 weeks | | | |
| AB1424/AB1612 F3′ TriNKET, | 47.2 | 46.1 | 6.8 |
| HST, pH 6.0, 40° C., 3 weeks | | | |
| AB1424/AB1612 F3′ TriNKET, | 48.7 | 44.3 | 7.0 |
| HST, pH 6.0, 40° C., 4 weeks | | | |

### Target Binding and Potency

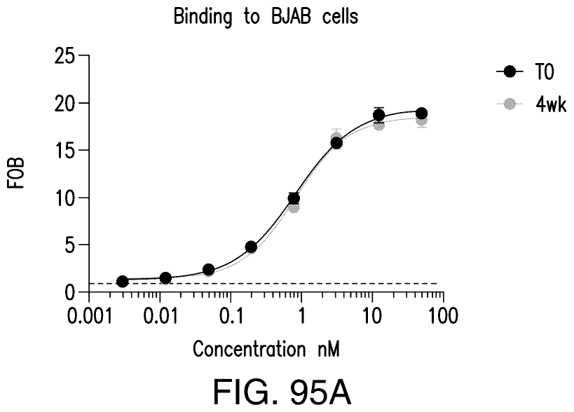

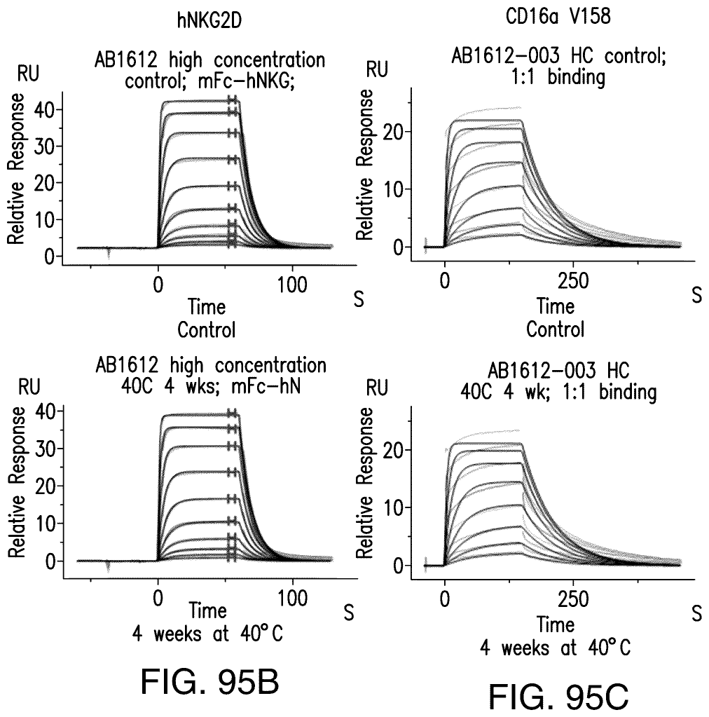

There was no meaningful difference observed in binding to all three intended targets (BAFF-R, NKG2D and CD16a) between control and stressed samples (FIG. 95A, FIG. 95B, and Table 66).

TABLE 66
Kinetic Parameters and Binding Affinities of High
Concentration AB1424/AB1612 F3′ TriNKET for
hNKG2D, hCD16a V158, and EC50 for Cell Expressed
hBAFF-R After 4 weeks at 40° C. in HST, pH 6.0.

| | | | | | K_{D} (nM) |
|---|---|---|---|---|---|
| | | | | K_{D} | Steady |
| Test Article | Target | kₐ (M⁻¹s⁻¹) | k_{d} (s⁻¹) | (nM) | State |
| AB1424/AB1612 F3′ | hNKG2D | (2.2 ± 0.0)*10⁵ | (1.1 ± 0.0)*10⁻¹ | 502.9 ± 5.9 | 544.3 ± 8.3 |
| TriNKET, HST, pH 6.0 | | | | | |
| Control | | | | | |
| AB1424/AB1612 F3′ | hNKG2D | (2.1 ± 0.1)*10⁵ | (1.0 ± 0.0)*10⁻¹ | 492.6 ± 9.7 | 521.9 ± 15.7 |
| TriNKET, HST, pH 6.0 | | | | | |
| 40° C., 4 weeks | | | | | |
| AB1424/AB1612 F3′ | hCD16aV | (1.3 ± 0.3)*10⁵ | (1.7 ± 0.0)*10⁻² | 144.8 ± 40.3 | N/A |
| TriNKET, HST, pH 6.0 | | | | | |
| Control | | | | | |
| AB1424/AB1612 F3′ | hCD16aV | (1.2 ± 0.4)*10⁵ | (1.6 ± 0.0)*10⁻² | 144.7 ± 40.4 | N/A |
| TriNKET, HST, pH 6.0 | | | | | |
| 40° C., 4 weeks | | | | | |

| | | EC₅₀ (nM) |
|---|---|---|
| AB1424/AB1612 F3′ | hBAFF-R | 0.82 |
| TriNKET, HST, pH 6.0 | | |
| Control | | |
| AB1424/AB1612 F3′ | hBAFF-R | 0.83 |
| TriNKET, HST, pH 6.0 | | |
| 40° C., 4 weeks | | |

n ≥ 3 replicates

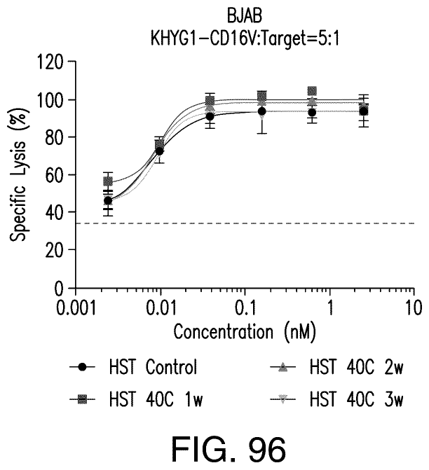

No difference in potency, quantified as percent cell lysis in a KHYG-1-CD16aV mediated cytotoxicity assay, was detected between the control and stressed samples (FIG. 96 and Table 67).

TABLE 67
Summary of High Concentration AB1424/AB1612 F3′ TriNKET
EC₅₀ and Maximum Lysis After 4 Weeks in HST, pH 6.0.

| Test article | EC₅₀ (nM) | Max killing (%) |
|---|---|---|
| AB1424/AB1612 F3′ | 0.01 | 94 |
| TriNKET, HST, pH 6.0, | | |
| Control | | |
| AB1424/AB1612 F3′ | 0.01 | 100 |
| TriNKET, HST, pH 6.0, | | |
| 40° C., 1 week | | |
| AB1424/AB1612 F3′ | 0.01 | 99 |
| TriNKET, HST, pH 6.0, | | |
| 40° C., 2 weeks | | |
| AB1424/AB1612 F3′ | 0.01 | 94 |
| TriNKET, HST, pH 6.0, | | |
| 40° C., 3 weeks | | |
| AB1424/AB1612 F3′ | 0.03 | 92 |
| TriNKET, HST, pH 6.0, | | |
| 40° C., 4 weeks | | |

### Example 8—Molecular Analysis of AB1424/AB1612 F4 TriNKET Format

In this Example, the molecular format, design, structure, and characteristics of AB1424/AB1612 F4 TriNKET were analyzed. These studies a) provided basic biochemical and biophysical characterization of the molecule, b) determined affinities of AB1424/AB1612 F4 TriNKET for BAFF-R, NKG2D, CD16a, a panel of FcγRs, and FcRn, c) confirmed binding of AB1424/AB1612 F4 TriNKET to BAFF-R+ cancer cells, d) demonstrated the selectivity of AB1424/AB1612 F4 TriNKET, e) determined the potency of AB1424/AB1612 F4 TriNKET in killing BAFF-R+ cancer cells, and f) evaluated the structural and functional integrity of AB1424/AB1612 F4 TriNKET following exposure to thermal, chemical, and mechanical stress.

AB1424/AB1612 F4 TriNKET is an F4 format TriNKET. AB1424/1612 F4 TriNKET is sometimes referred to herein as AB1426. AB1424/1612 F4 TriNKET (AB1424/1612-F4) includes four polypeptides: a first polypeptide comprising AB1424/1612-VH-CH1-CH2-CH3-A49MI-scFv (SEQ ID NO:271) (“Chain M”), a second polypeptide comprising AB-1424/1612-VH-CH1-CH2-CH3 (SEQ ID NO:272) (“Chain H”), and a third and fourth polypeptide each comprising AB1424/1612-VL-CL (SEQ ID NO:273) (“Chain L”).

### Molecular Modeling

Anti-BAFF-R and anti-NKG2D binding arms of AB1424/AB1612 F4 TriNKET were compared with 377 post Phase I biotherapeutic molecules using Therapeutic Antibody Profiler (TAP) available at the SAbPred website. TAP used ABodyBuilder to generate a model for AB1424/AB1612 with side chains by PEARS. The CDRH3 was built by MODELLER due to its diversity.

Five different parameters were evaluated:
Total CDR length
Patches of surface hydrophobicity (PSH) across the CDR vicinity
Patches of positive charge (PPC) across the CDR vicinity
Patches of negative charge (PNC) across the CDR vicinity
Structural Fv charge symmetry parameter (sFvCSP)

These parameters of AB1424/AB1612 F4 TriNKET were then compared with the profile distributions of therapeutic antibodies to predict the developability and any potential issues that might cause downstream challenges.

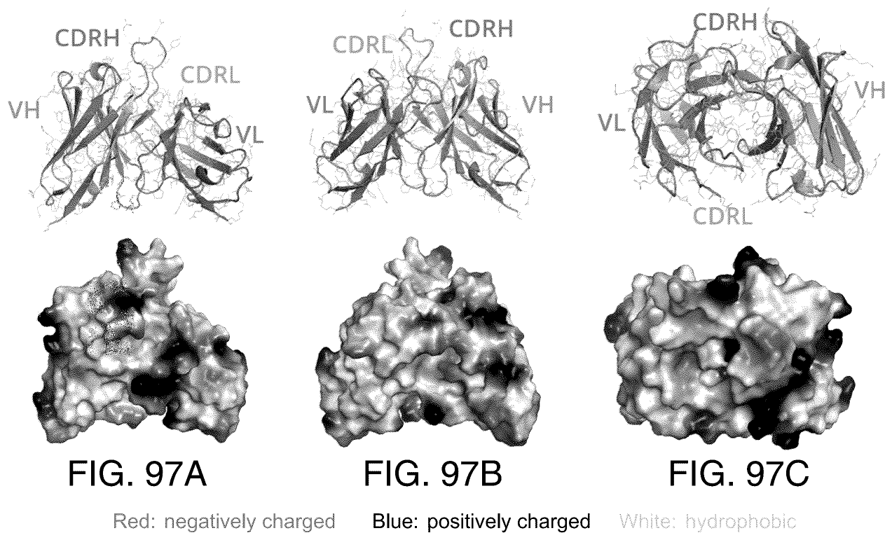

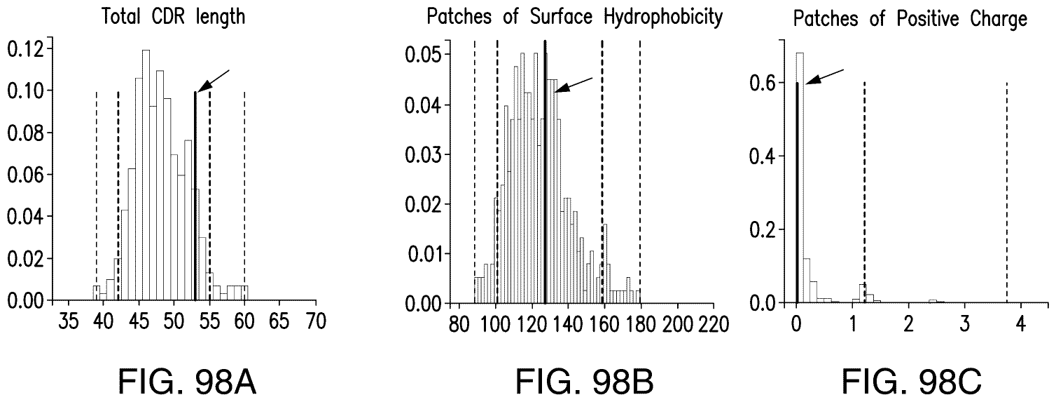

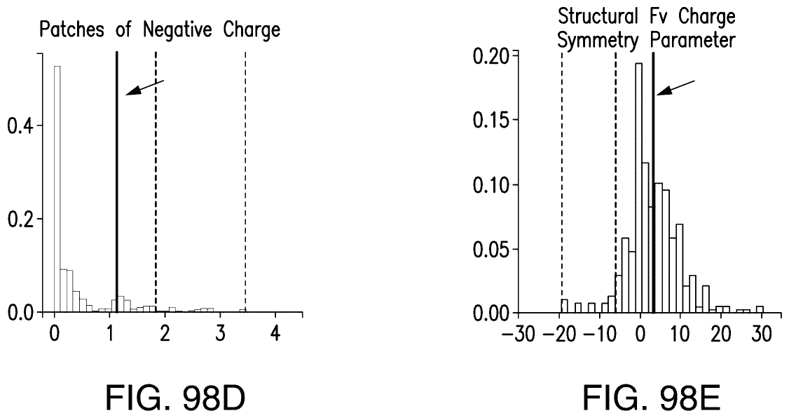

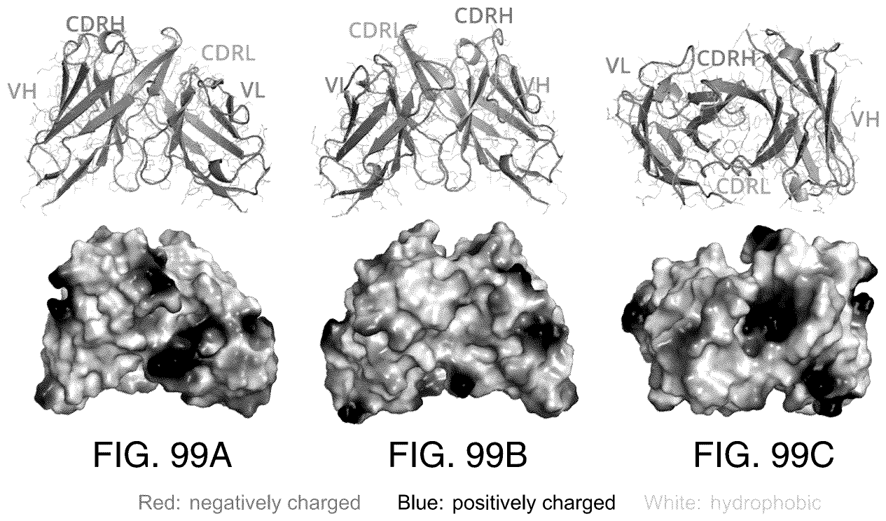

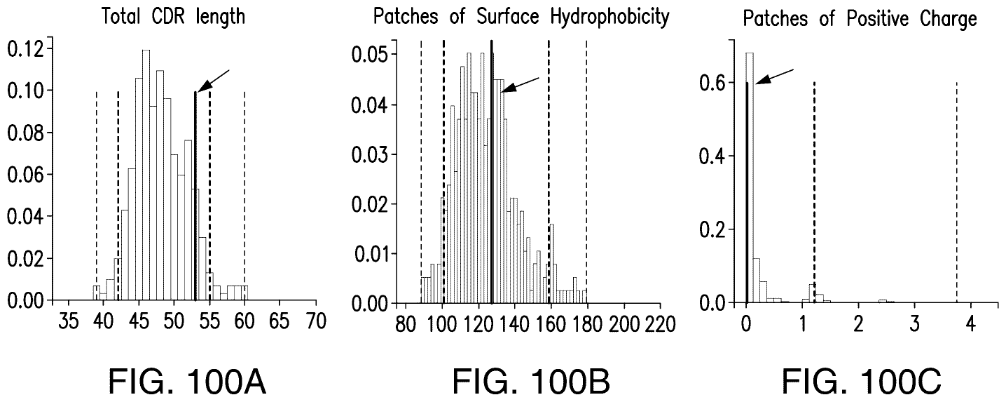

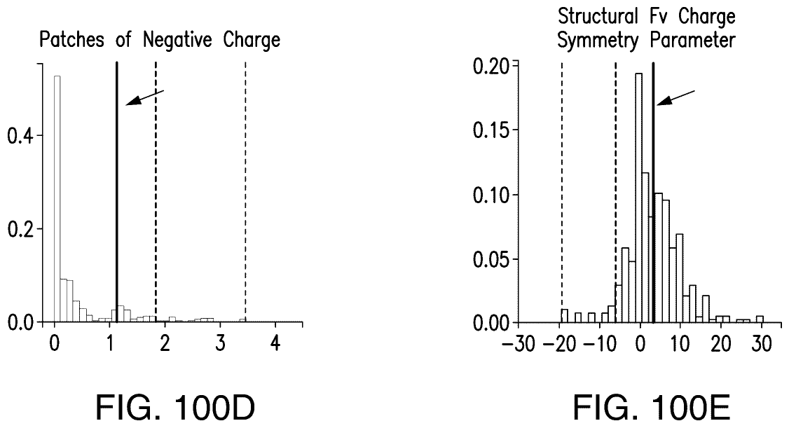

FIG. 97A-FIG. 97C is a model of the variable domains of the BAFF-R Fab binding arm of AB1424/AB1612 F4 TriNKET in three different orientations (upper panel) and the corresponding surface charge distribution of the same orientation (lower panel). The surface charge distribution of the CDR interface is predominately negatively charged (“top view,” lower panel) with some clusters of hydrophobic residues. The surface charge distribution of the BAFF-R arm was evenly distributed across the modeled paratope. Hydrophobic patch analysis of the BAFF-R binding arm of AB1424/AB1612 F4 TriNKET benchmarks with the vast majority of therapeutic mAbs (FIG. 98A-FIG. 98E). Surface patches of positive and negative charge have been associated with adverse impacts on mAb expression and accelerated in vivo clearance. For the BAFF-R binding arm of AB1424/AB1612 F4 TriNKET, the positively charged patches, negatively charged patches, and charge symmetry are akin to the majority of reference mAbs (FIG. 99A-FIG. 99C). The NKG2D binding arm was modeled and depicted in three different orientations and their corresponding surface charge distribution is shown (FIG. 99A-FIG. 99C). The surface charge distribution of the NKG2D arm was evenly distributed across the modeled paratope. FIG. 100A-FIG. 100E show the total CDR length and surface feature analyses of the NKG2D binding arm of AB1424/AB1612 F4 TriNKET. In summary, there were neither unusual surface charge properties nor unusual patches of surface hydrophobicity identified.

### AB1424/AB1612 F4 TriNKET Expression and Purification

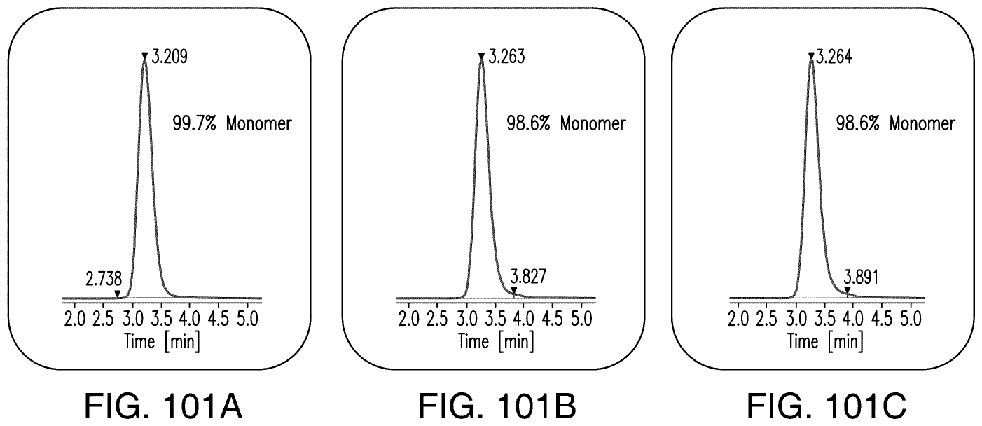

AB1424/AB1612 F4 TriNKET was expressed in ExpiCHO cells and purified. The purity of AB1424/AB1612 F4 TriNKET was determined by size exclusion chromatography (SEC) and capillary electrophoresis sodium dodecyl sulfate (CE-SDS). AB1424/AB1612 F4 TriNKET exhibited high monomer content (>98.6% as shown in FIG. 101A-FIG. 101C), and no major impurities were observed under CE-SDS. The purity of three lots of AB1424/AB1612 F4 TriNKET as determined by SEC and CE-SDS is summarized in Table 68.

TABLE 68
Purity analysis of AB1424/AB1612 F4 TriNKET by SEC and CE-SDS.

| | SEC | CE-SDS |
|---|---|---|
| Test article | % monomer | % purity |
| AB1424/AB1612 F4 TriNKET | 99.7 | 99.9 |
| AB1424/AB1612 F4 TriNKET | 98.6 | 99.7 |
| AB1424/AB1612 F4 TriNKET | 98.6 | 99.7 |

Charge Profile Analysis by cIEF

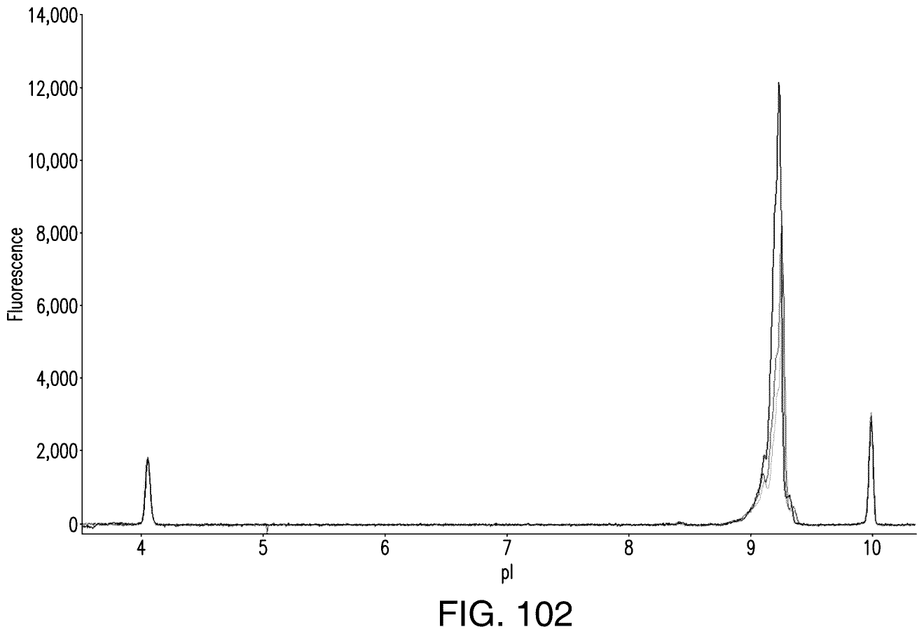

The charge profile of AB1424/AB1612 F4 TriNKET was analyzed by capillary isoelectric focusing (cIEF) (FIG. 102 and Table 69). AB1424/AB1612 F4 TriNKET showed a major peak at a pI of 9.3. Several less abundant, overlapping acidic peaks and minor basic peaks were also observed.

TABLE 69
Charge profile results of AB1424/AB1612 F4 TriNKET by cIEF.

| Test Article | pI | % Acidic | % Main | % Basic |
|---|---|---|---|---|
| AB1424/AB1612 F4 | 9.3 | 44.4 | 52.2 | 3.4 |
| TriNKET | | | | |
| AB1424/AB1612 F4 | 9.3 | 52.7 | 44.2 | 3.1 |
| TriNKET | | | | |
| AB1424/AB1612 F4 | 9.3 | 51.3 | 45.3 | 3.3 |
| TriNKET | | | | |

### Hydrophobic Interactions Chromatography

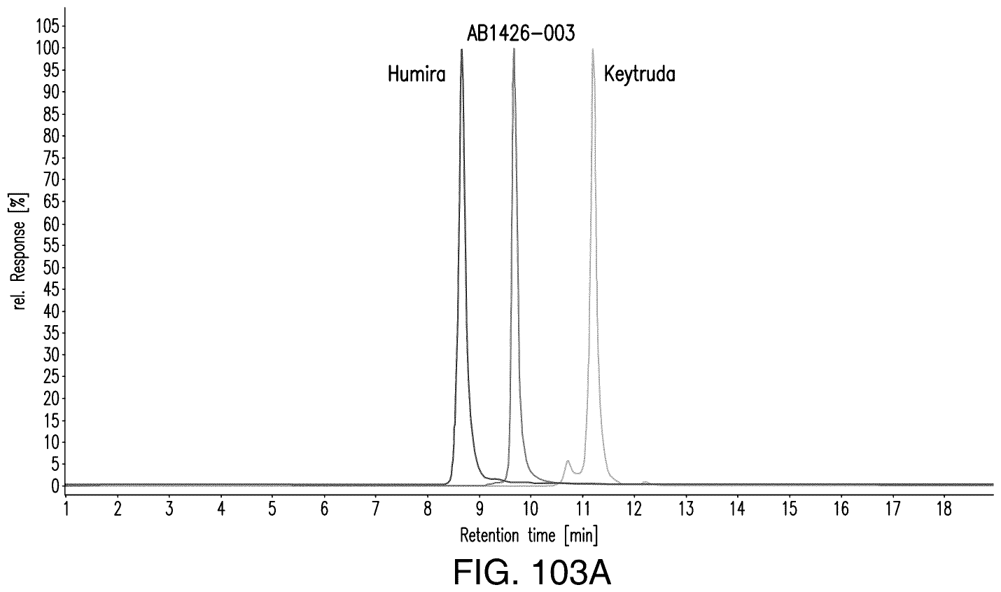

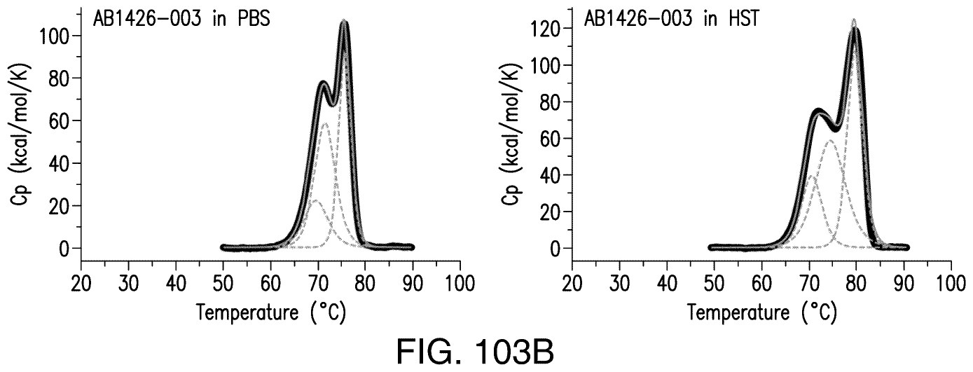

The hydrophobicity prediction data was confirmed by investigating AB1424/AB1612 F4 TriNKET behavior with analytical Hydrophobic Interactions Chromatography (HIC), a technique that relies upon proteins with significant patches of exposed hydrophobic patches being more prone to aggregation. HIC was performed as described in Example 4 above. Retention times of AB1424/AB1612 F4 TriNKET on the analytical HIC column is shown in Table 70 and HIC profile in FIG. 103A. Commercial adalimumab and pembrolizumab were used as examples of well-behaved biologics and functioned as internal controls for the assay. AB1424/AB1612 F4 TriNKET has a retention time of 9.7 minutes, compared to 11.2 minutes for pembrolizumab and 8.7 for adalimumab. Thus, experimental hydrophobicity analysis suggests that the hydrophobic properties of AB1424/AB1612 F4 TriNKET are acceptable for further development.

TABLE 70
HIC analysis of AB1424/AB1612 F4 TriNKET

| | Retention | Retention time relative to |
|---|---|---|
| Test article | time (min) | Adalimumab/Humira (min) |
| Adalimumab/Humira | 8.7 | N/A |
| Pembrolizumab/Keytruda | 11.2 | +2.5 |
| AB1424/AB1612 F4 | 9.7 | +1.0 |
| TriNKET | | |

### Thermal Stability Analysis

The thermal stability of AB1424/AB1612 F4 TriNKET was assessed by differential scanning calorimetry (DSC) in PBS pH 7.4 or in HST comprising 20 mM histidine, 250 mM sucrose, 0.01% tween-80 at pH 6.0. DSC was performed as described in Example 4 above.

AB1424/AB1612 F4 TriNKET demonstrated high thermal stability in both buffers (FIG. 103B and Table 71).

TABLE 71
Thermal stability of AB1424/AB1612 F4 TriNKET

| | | Tₒₙₛₑₜ | Tₘ₁ | Tₘ₂ | Tₘ₃ |
|---|---|---|---|---|---|
| Test article | Buffer | (° C.) | (° C.) | (° C.) | (° C.) |
| AB1424/AB1612 | PBS, pH 7.4 | 62.5 | 69.3 | 71.2 | 75.4 |
| F4 TriNKET | | | | | |
| AB1424/AB1612 | HST, pH 6.0 | 63.3 | 70.3 | 74.2 | 79.4 |
| F4 TriNKET | | | | | |

### Disulfide Bond Arrangement

AB1424/AB1612 F4 TriNKET was constructed as an engineered molecule based on the backbone of a monoclonal IgG1 antibody. While a typical IgG1 contains 16 disulfide bonds, the F4 format of AB1424/AB1612 F4 TriNKET was constructed with 20 disulfide bonds.

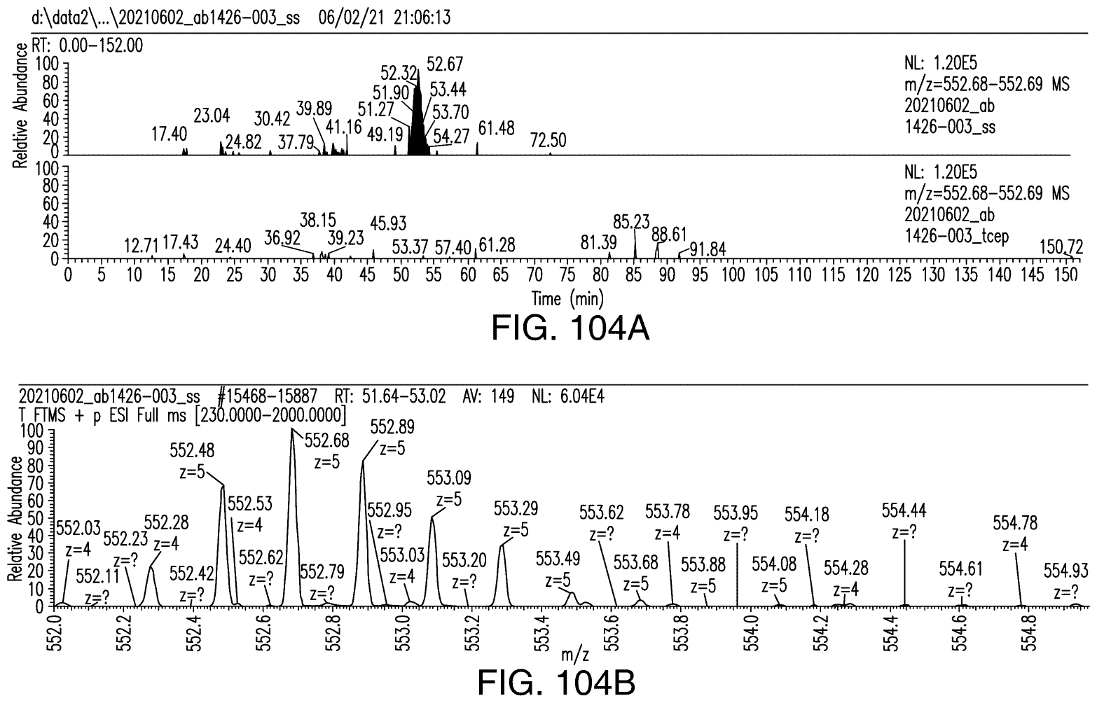

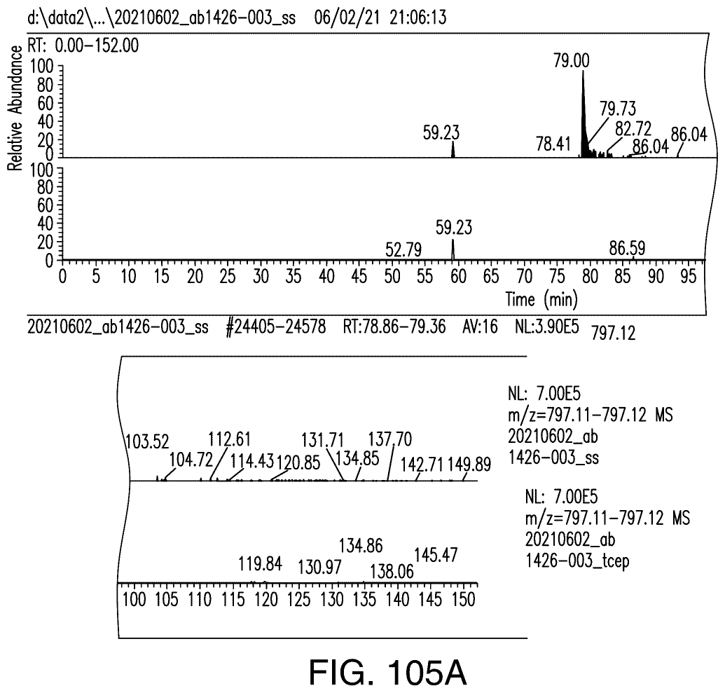

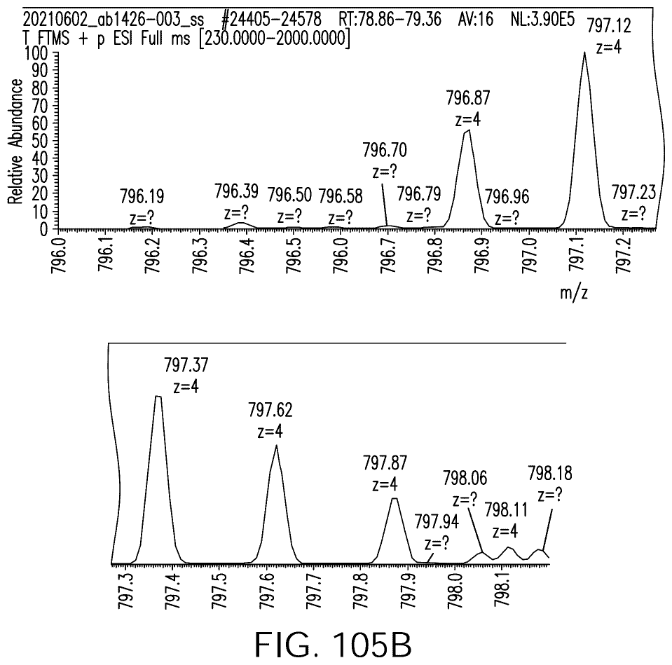

The disulfide bond arrangement of AB1424/AB1612 F4 TriNKET was confirmed by LC-MS/MS peptide mapping analysis of a non-reduced tryptic digest. Disulfide bonded peptides were identified by MS/MS database searching and confirmed by comparing their intensities in the native and reduced digests. All of the standard disulfides expected from the antibody structure were confirmed. FIG. 104A and FIG. 104B show extracted ion chromatograms (XICs) for the engineered disulfide pair in the Fc (non-reduced and reduced) and the most intense charge state for that peptide pair. Similarly, XICs in FIG. 105A and FIG. 105B confirm the existence of the engineered disulfide bridge introduced to stabilize the scFv. A summary of the observed disulfide linked peptides in AB1424/AB1612 F4 TriNKET is shown in Table 72. All theoretical disulfide linked peptides were observed with high mass accuracy (<2.0 ppm), were reducible, and were sequence-confirmed by MS/MS fragmentation.

TABLE 72
Disulfide linked peptide theoretical and experimental mass.

| | | | Theo- | Experi- | Mass |
|---|---|---|---|---|---|
| | | | retical | mental | accuracy |
| Chain | Domain | Peptide | mass (Da) | mass (Da) | (ppm) |
| L | VL | L2:L7 | 5147.4056 | 5147.4132 | 1.4 |
| | CL | L11:L18 | 3555.7490 | 3555.7490 | 0.4 |
| H | VH | H3:H11 | 3408.4638 | 3408.4599 | −1.0 |
| | CH1 | H16:H17-18 | 7916.9194 | 7916.9058 | −1.5 |
| | CH2 | H26:H33 | 2328.0977 | 2328.0961 | −0.7 |
| | CH3 | H40:H44 | 4432.0675 | 4432.0636 | −0.9 |
| M | VH | M3:M11 | non-unique, see H3:H11 | | |
| | CH1 | M16:M17-18 | non-unique, see H16:H17-18 | | |
| | CH2 | M26:M33 | non-unique, see H26:H33 | | |
| | CH3 | M42:M47 | 3844.8236 | 3844.8302 | 1.4 |
| | scFv VL | M49:M54 | 4482.0784 | 4482.0759 | −0.4 |
| | scFv | M55:M61* | 3183.4417 | 3183.4433 | 0.8 |
| | stabilizing* | | | | |
| | scFv VH | M59:M66 | 3371.4686 | 3371.4708 | 0.8 |
| Inter- | Hinge | H23-24:M23-24 | 5454.7834 | 5454.7855 | 0.5 |
| mole- | CL-CH1 | L19-20:H22 | 1260.4863 | 1260.4843 | −1.6 |
| cular | CL-CH1 | L19-20:M22 | non-unique, see L19-20:H22 | | |
| | CH3-CH3* | H39:M40-41* | 2757.3830 | 2757.3889 | 1.9 |

### Binding Characteristics of AB1424/AB1612 F4 TriNKET

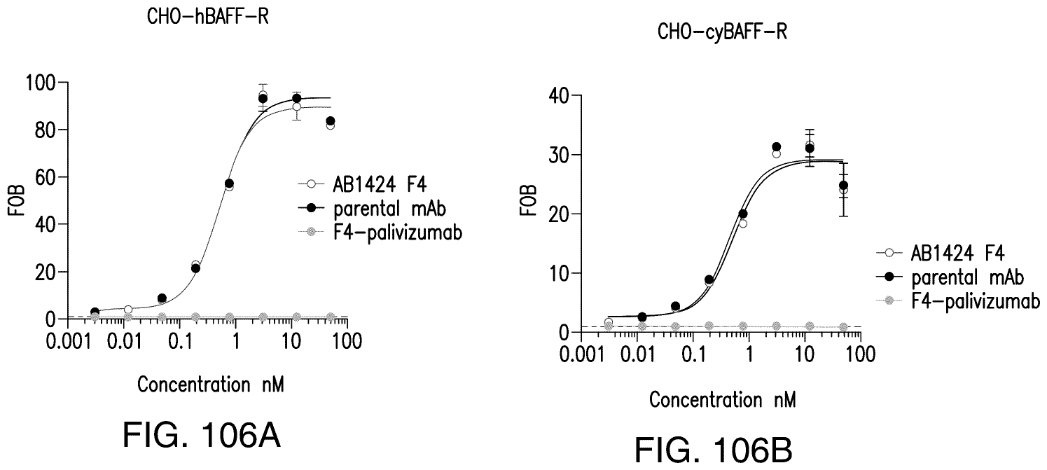

Isogenic cell lines overexpressing human and cynomolgus BAFF-R were developed from CHO cells. Binding of AB1424/AB1612 F4 TriNKET to the cell surface-expressed BAFF-R was compared to the parental BAFF-R specific antibody as well as to an F4 format control that does not contain a BAFF-R binder (F4-palivizumab). AB1424/AB1612 F4 TriNKET and its parental mAb demonstrated a similar dose-response in binding to human and cynomolgus BAFF-R (FIG. 106A and FIG. 106B). The EC₅₀ and maximal FOB was nearly identical for AB1424/AB1612 F4 TriNKET and parental mAb when comparing binding to human and cynomolgus BAFF-R (Table 73).

TABLE 73
Binding of AB1424/AB1612 F4 TriNKET and parental mAb
to human and cynomolgus cell surface expressed BAFF-R.

| | Human BAFF-R⁺ | | Cynomolgus BAFF-R⁺ | |
|---|---|---|---|---|
| | isogenic CHO | | isogenic CHO | |
| | EC₅₀ | Max | EC₅₀ | Max |
| Test article | (nM) | FOB | (nM) | FOB |
| AB1424/AB1612 | 0.37 ± 0.11 | 120 ± 86 | 0.51 ± 0.03 | 56 ± 52 |
| F4 TriNKET | | | | |
| Parental mAb | 0.39 ± 0.17 | 12 ± 85 | 0.57 ± 0.23 | 58 ± 50 |

Binding of AB1424/AB1612 F4 TriNKET to a subset of BAFF-R⁺ cancer cell lines was assessed by flow cytometry. AB1424/AB1612 F4 TriNKET bound with low nanomolar EC₅₀ to cell-surface BAFF-R on BJAB, Raji, RL, Rs4;11, Jeko-1 and SUDHL-6 cells. The EC₅₀ was comparable amongst the BAFF-R⁺ cancer cell lines (Table 74).

TABLE 74
Binding of AB1424/AB1612 F4 TriNKET
to BAFF-R⁺ human cancer cell lines

| Cell line | AB1424/AB1612 F4 TriNKET EC₅₀ (nM) |
|---|---|
| BJAB | 0.37 ± 0.16 |
| Raji | 0.13 ± 0.02 |
| RL | 0.17 ± 0.02 |
| Rs4; 11 | 0.08 ± 0.03 |
| Jeko-1 | 0.08 ± 0.03 |
| SUDHL-6 | 0.18 ± 0.04 |

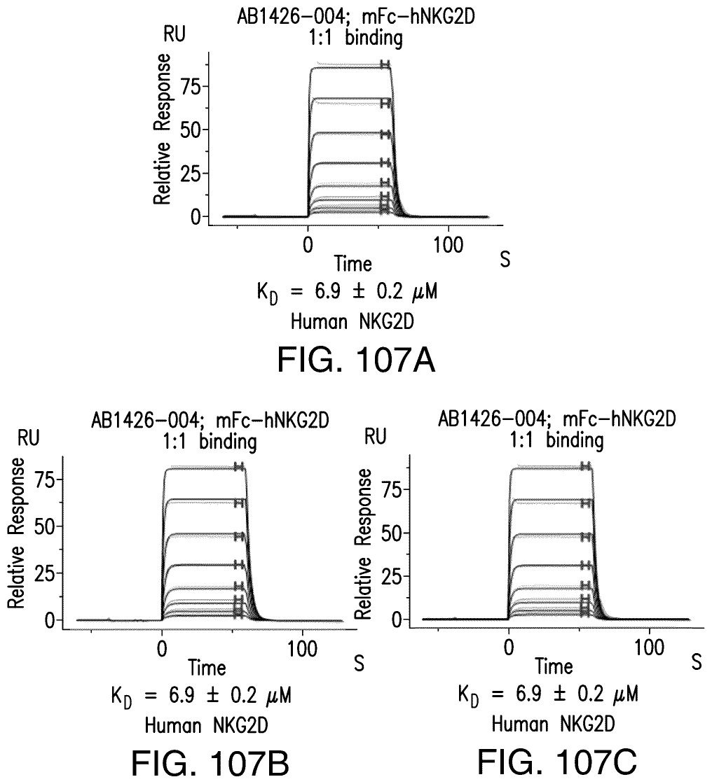

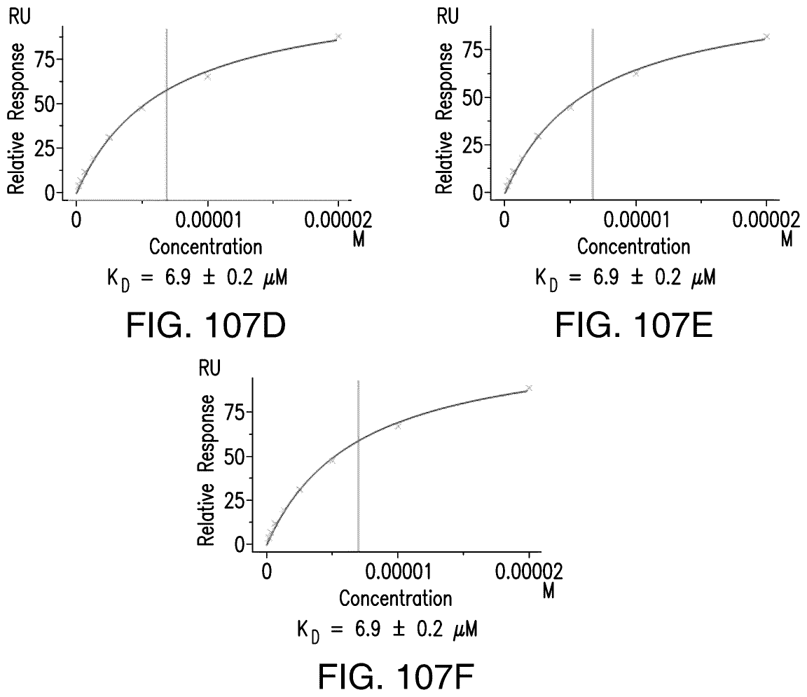

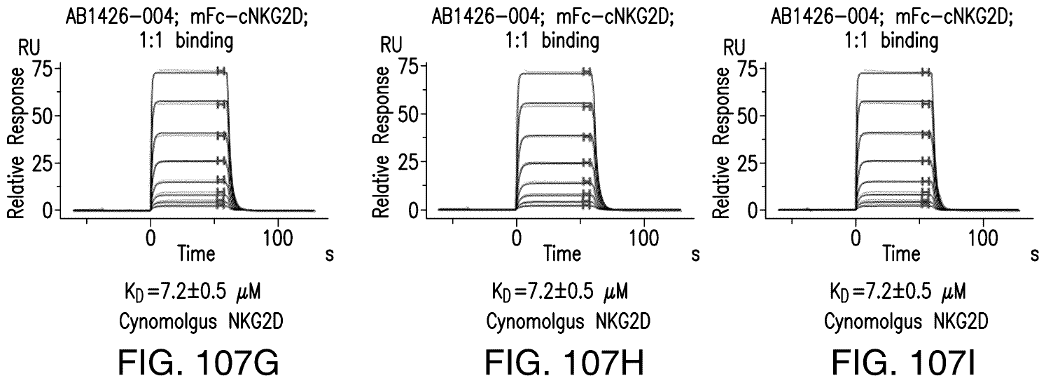

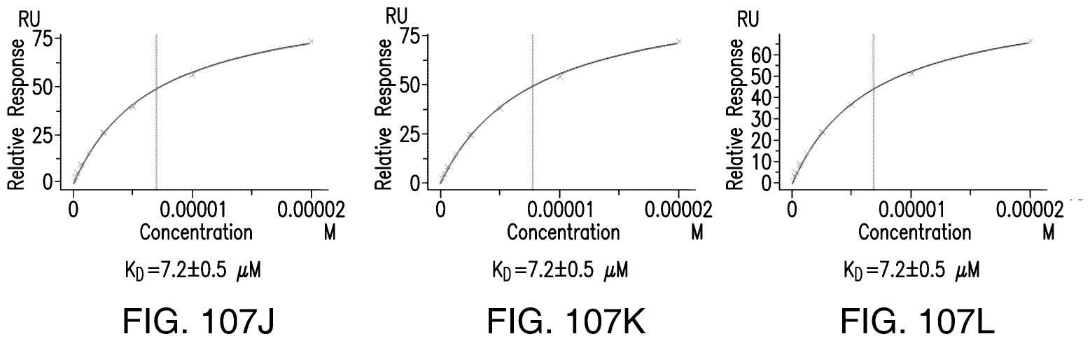

Binding of AB1424/AB1612 F4 TriNKET to human and cynomolgus NKG2D was assessed by SPR (FIG. 107A-FIG. 107L). NKG2D is a native dimer, therefore recombinant mFc-tagged NKG2D dimer was used for this experiment. The steady state affinities to human and cynomolgus NKG2D are shown in Table 75. The affinities of AB1424/AB1612 F4 TriNKET for human and cynomolgus NKG2D were comparable.

TABLE 75
Steady state affinity of AB1424/AB1612 F4
TriNKET for human NKG2D measured by SPR.

| | Affinity for human | Affinity for cyno |
|---|---|---|
| Test article | NKG2D K_{D} (μM) | NKG2D K_{D} (μM) |
| AB1424/AB1612 F4 | 6.9 | 7.0 |
| TriNKET | | |
| AB1424/AB1612 F4 | 6.7 | 7.7 |
| TriNKET | | |
| AB1424/AB1612 F4 | 7.0 | 6.9 |
| TriNKET | | |
| Average ± StDev | 6.9 ± 0.2 | 7.2 ± 0.5 |

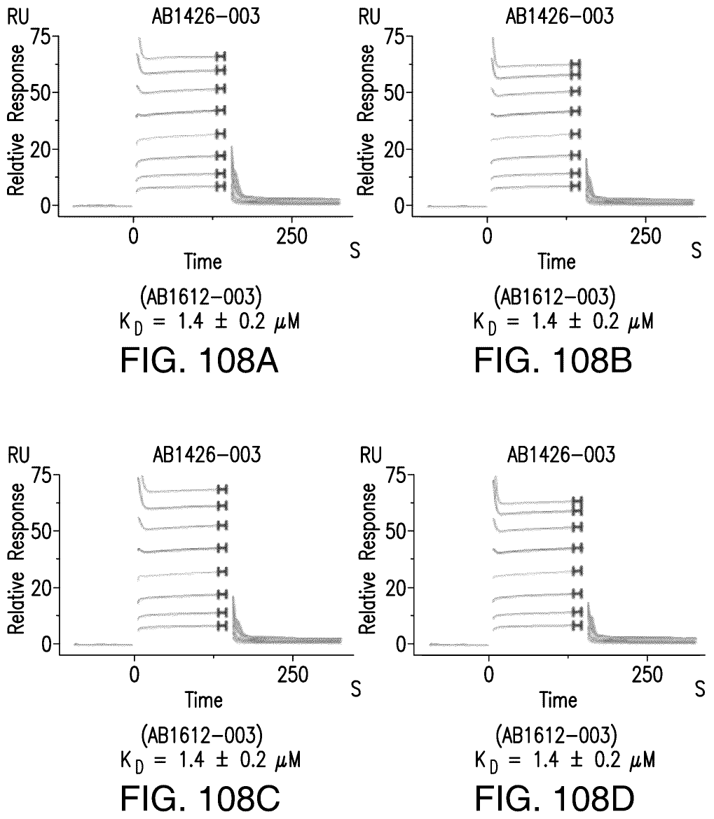

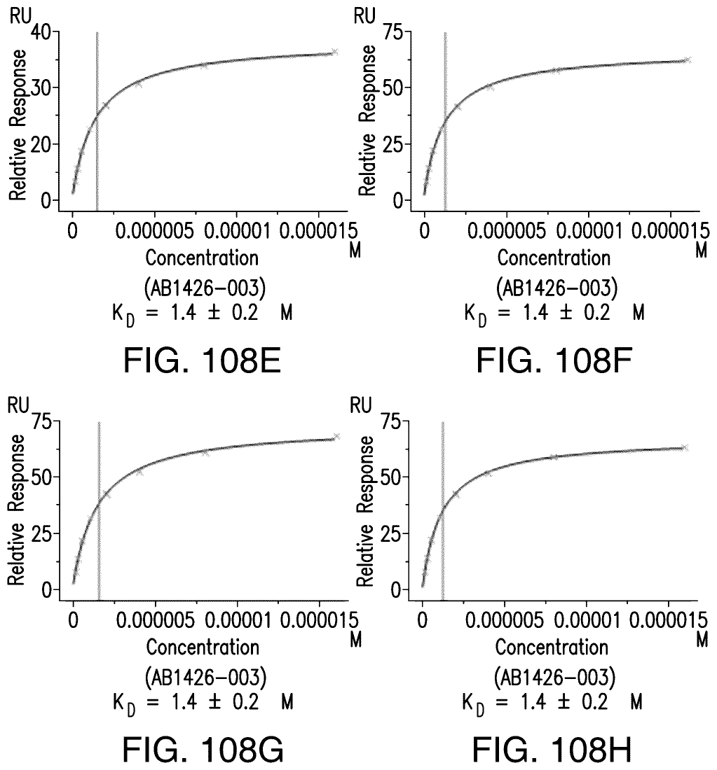

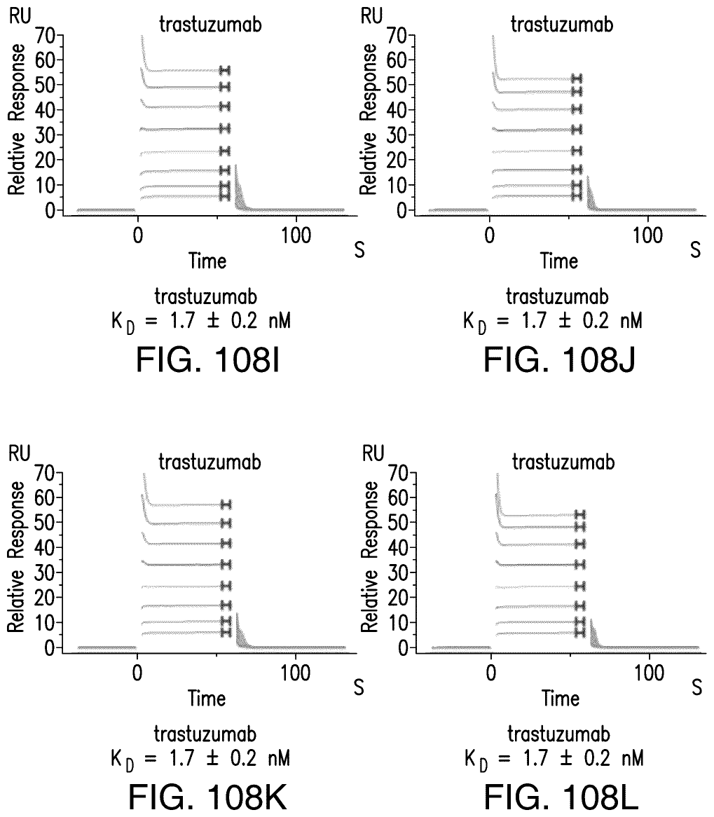

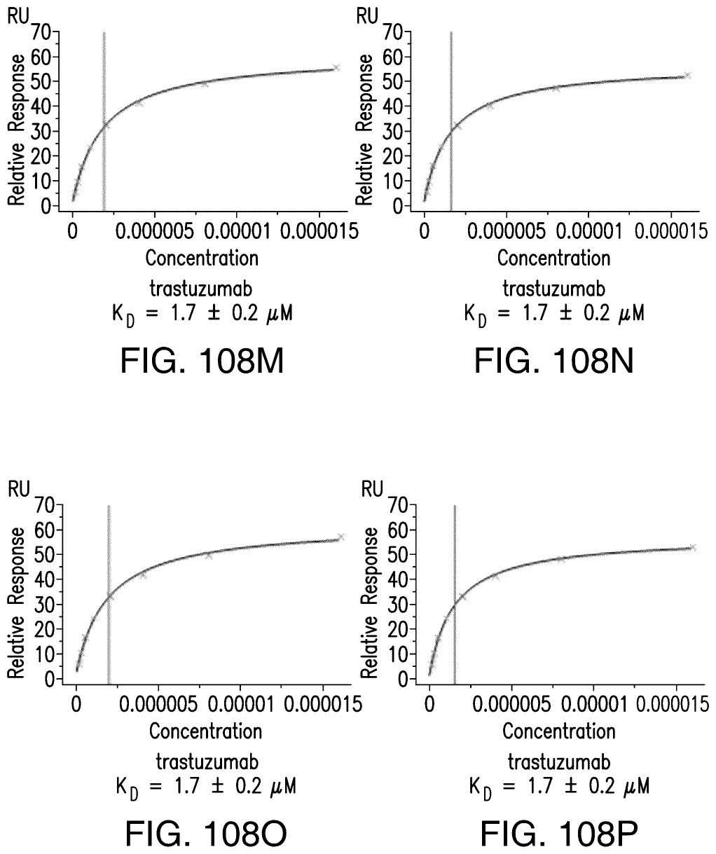

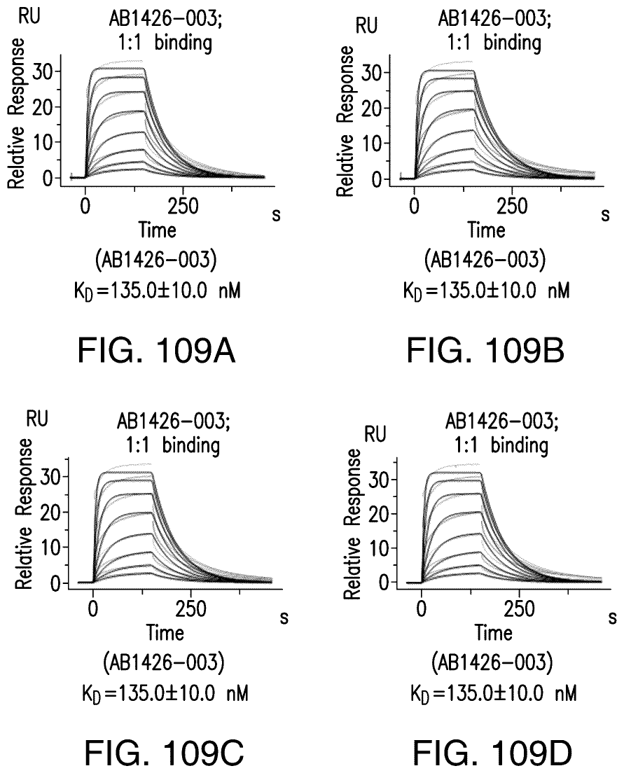

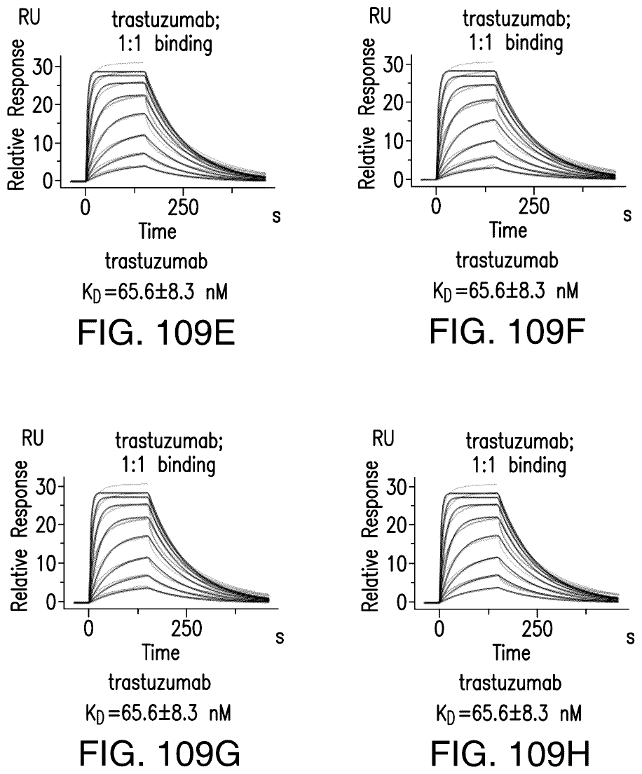

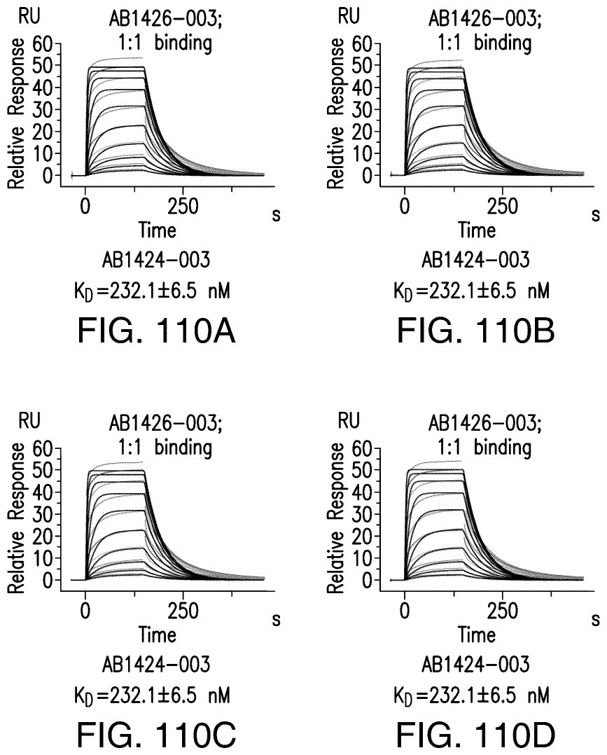

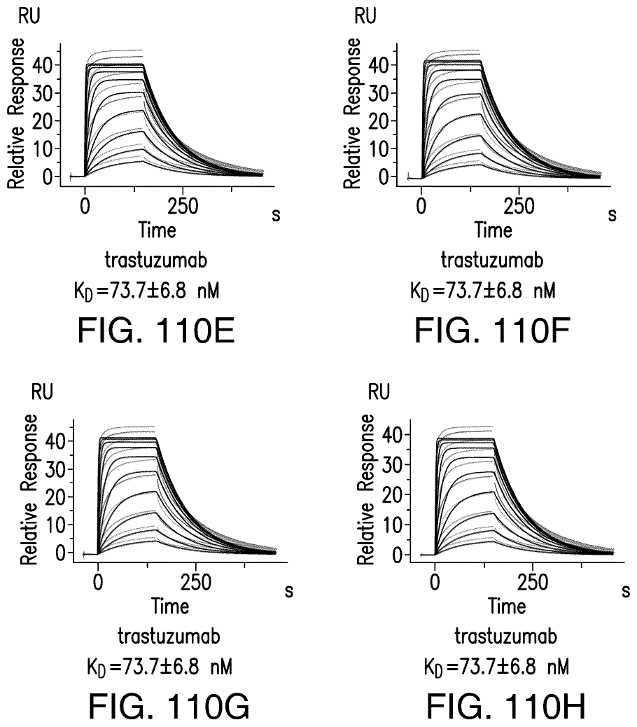

AB1424/AB1612 F4 TriNKET was constructed with a human IgG1 Fc meant to maintain interactions with Fc receptors. Engagement of CD16a is a key driver of the TriNKET mechanism of action. Binding to both human CD16a V158 and F158 alleles, as well as to cynomolgus CD16 was assessed as part of the full FcR panel analysis by SPR as presented in Table 76 and demonstrated that AB1424/AB1612 F4 TriNKET binds human and cynomolgus CD16 comparably to IgG1 isotype control trastuzumab. FIG. 108A-FIG. 108P, FIG. 109A-FIG. 109H, and FIG. 110A-FIG. 110H represent raw data and fitted sensorgrams for CD16a V158, F158 and cynomolgus CD16, respectively. As such, AB1424/AB1612 F4 TriNKET demonstrates good binding to CD16.

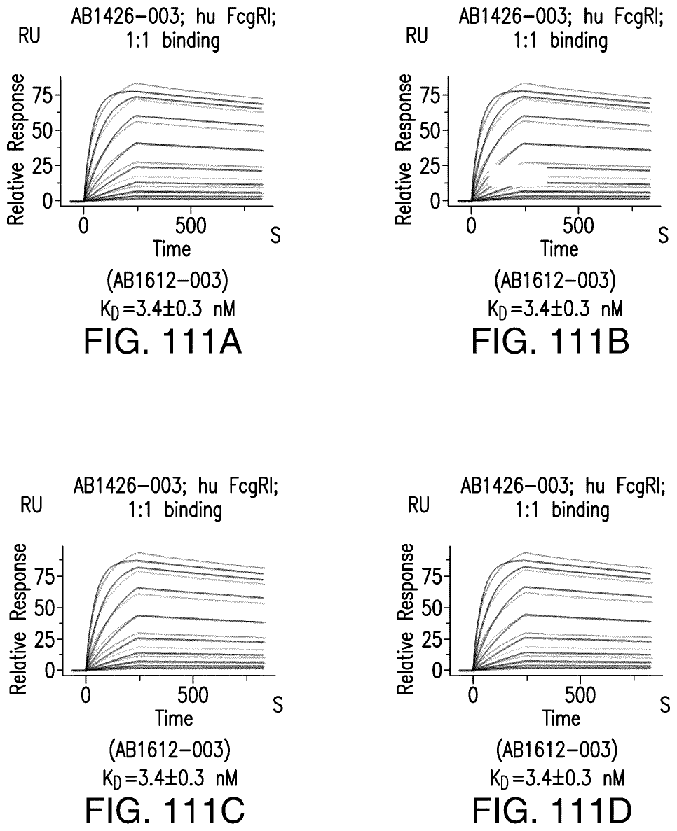

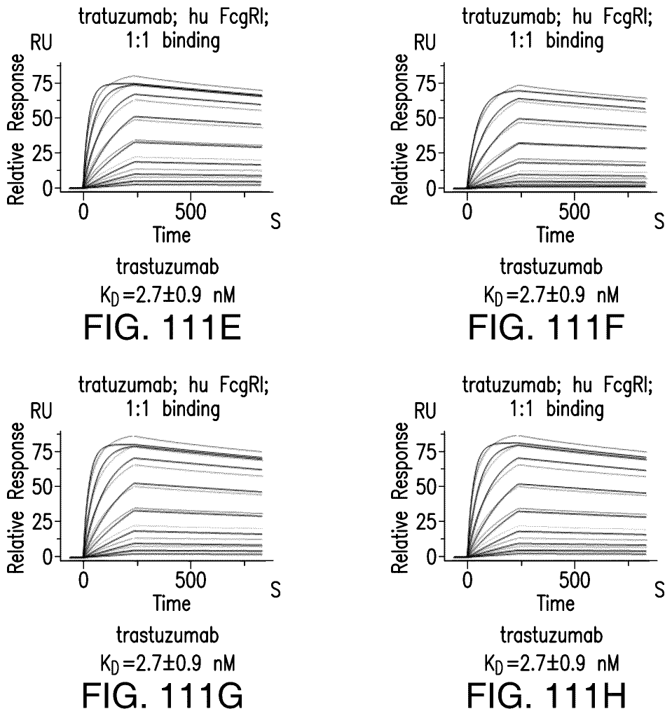

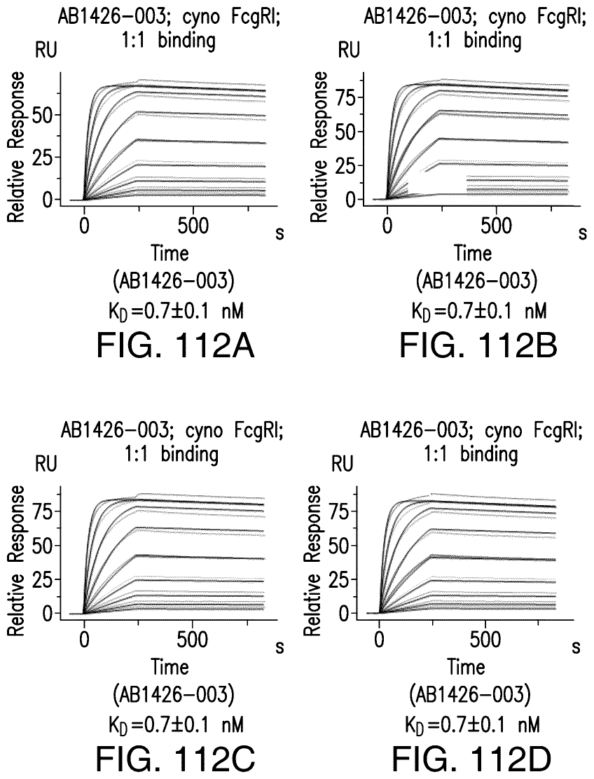

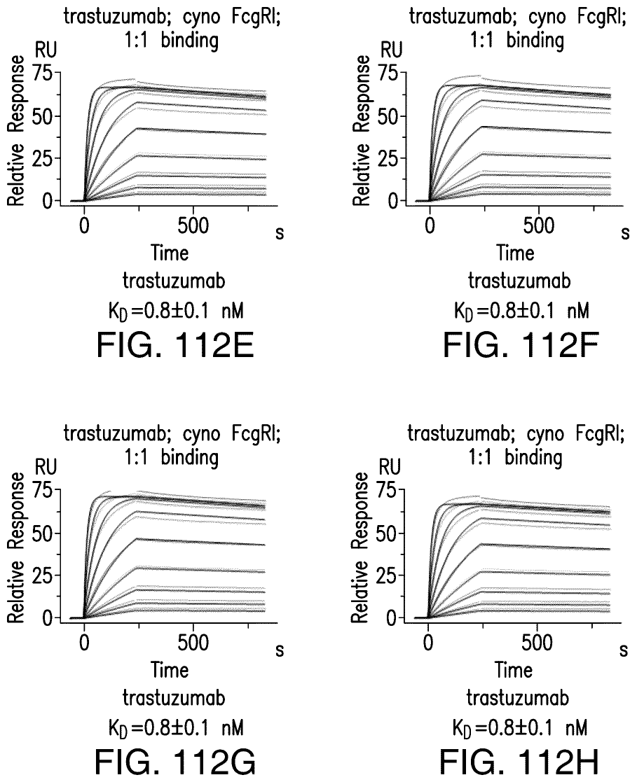

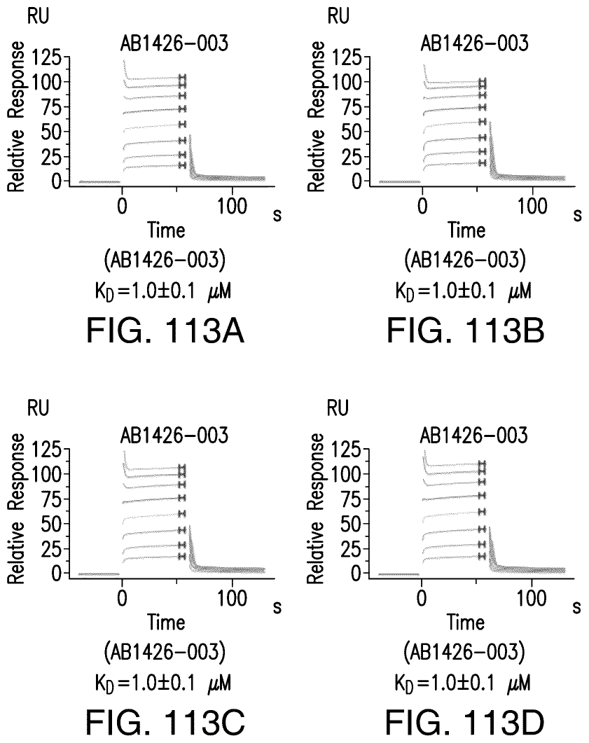

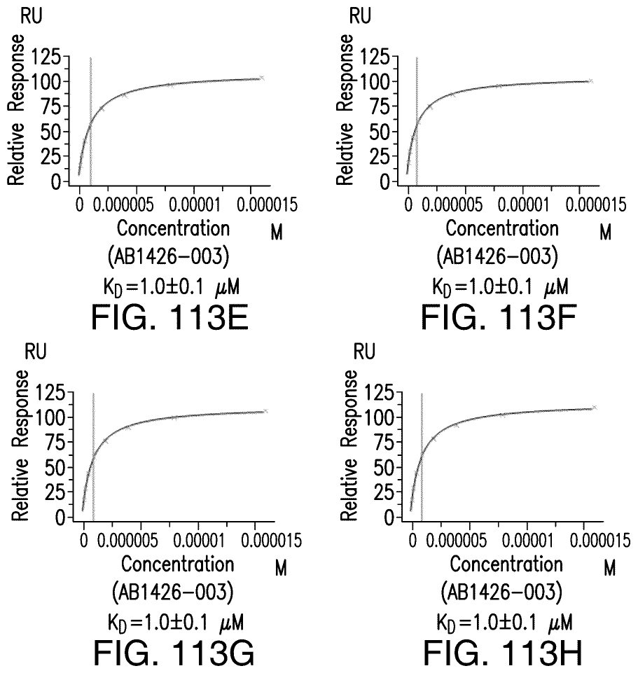

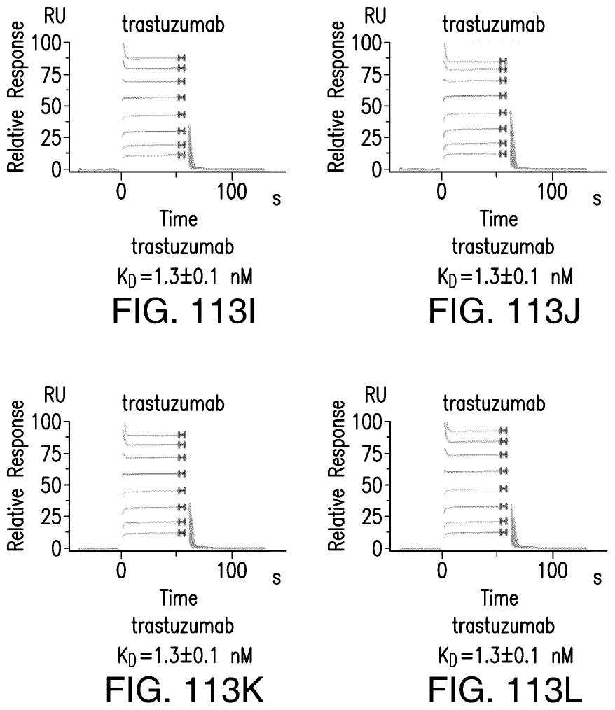

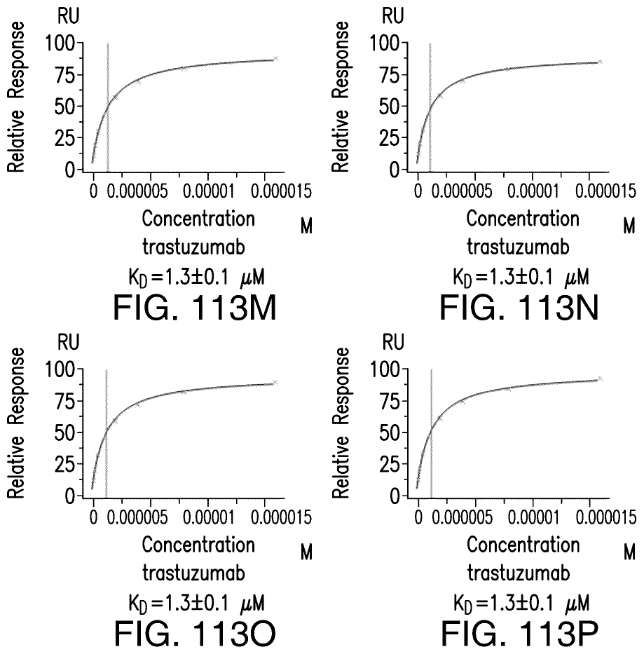

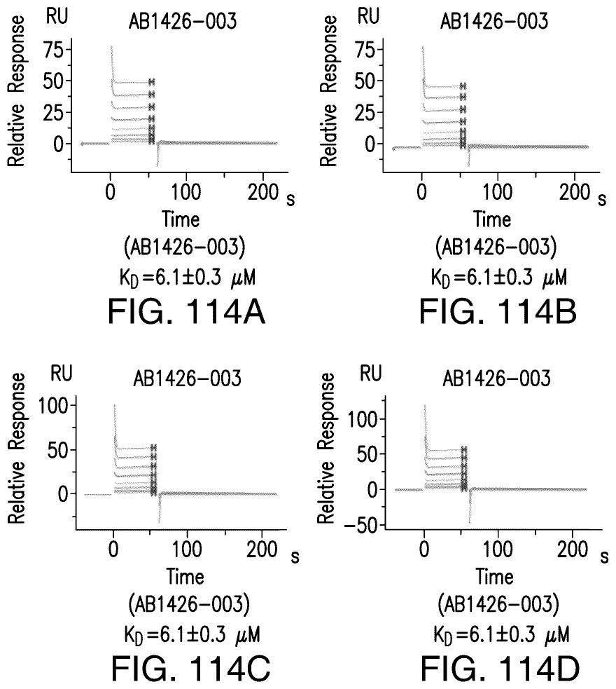

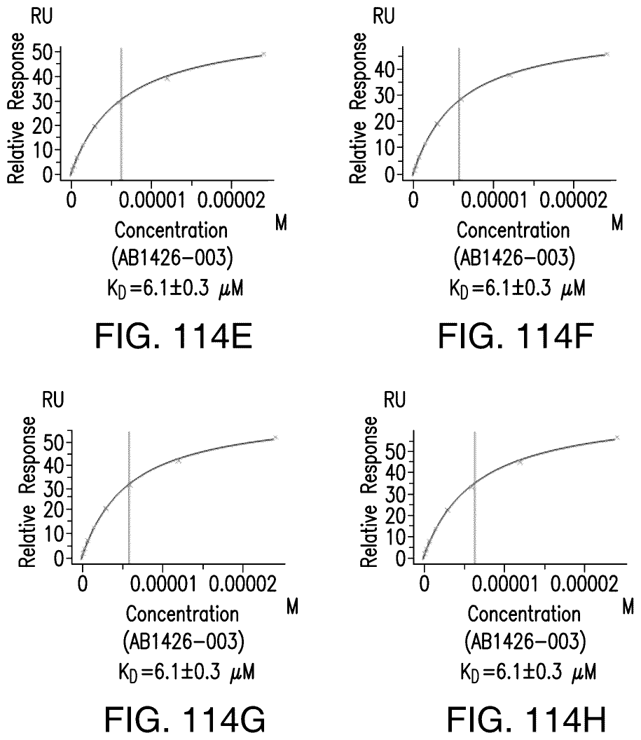

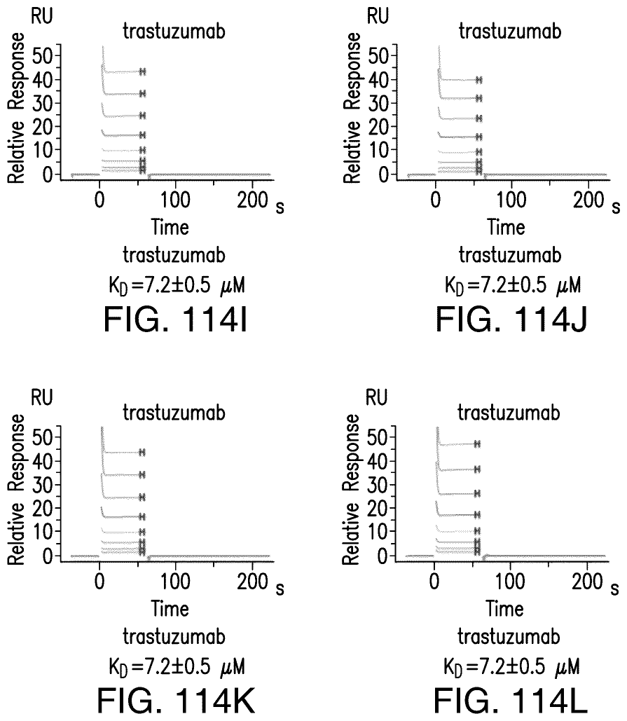

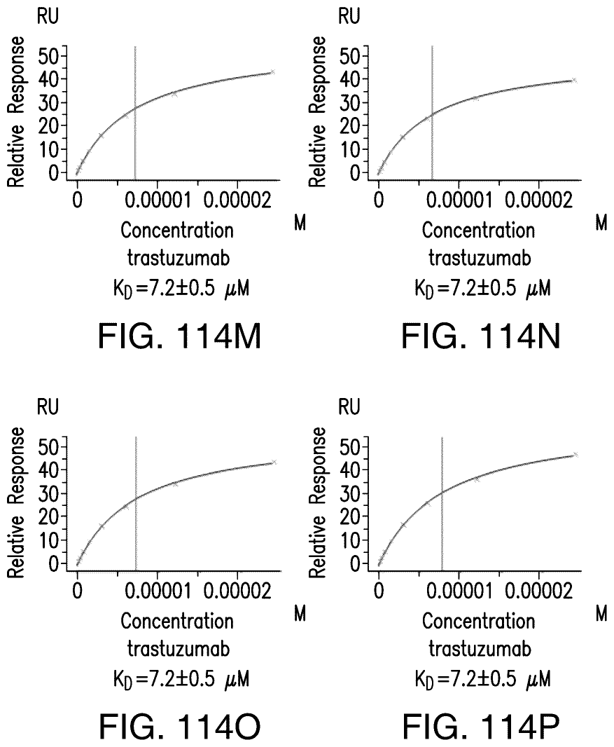

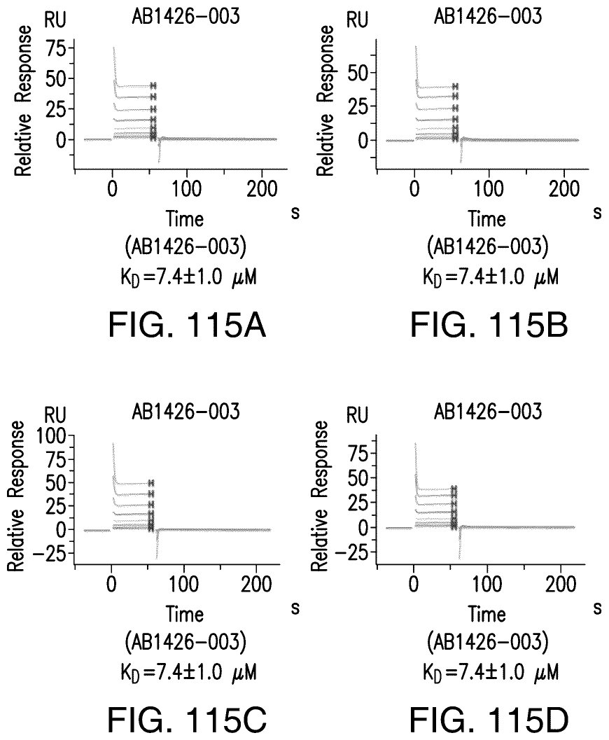

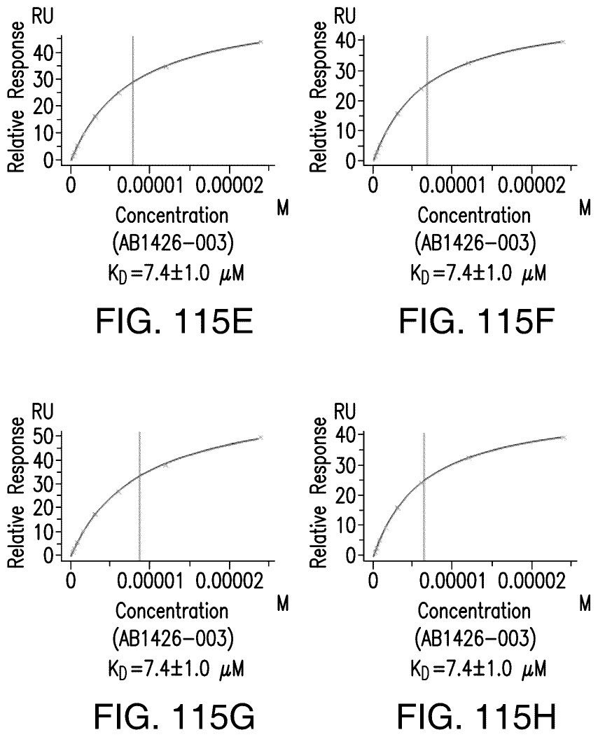

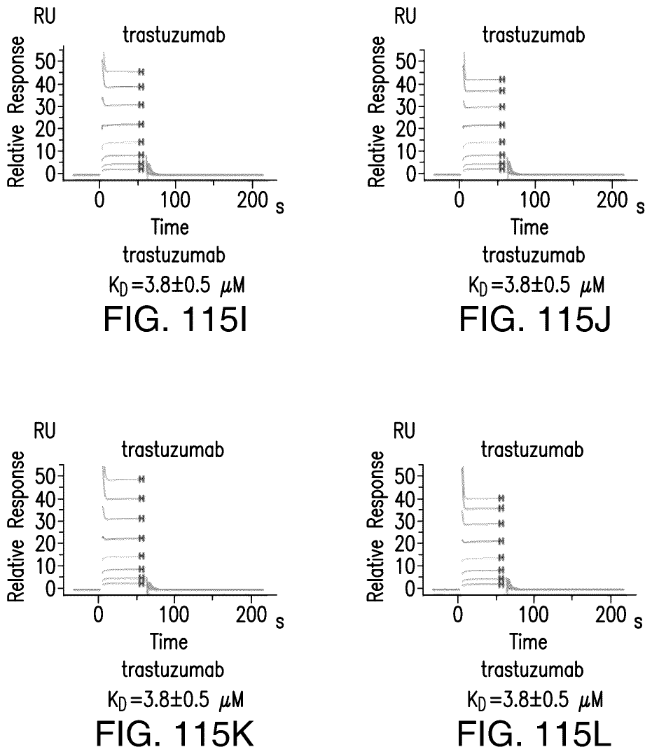

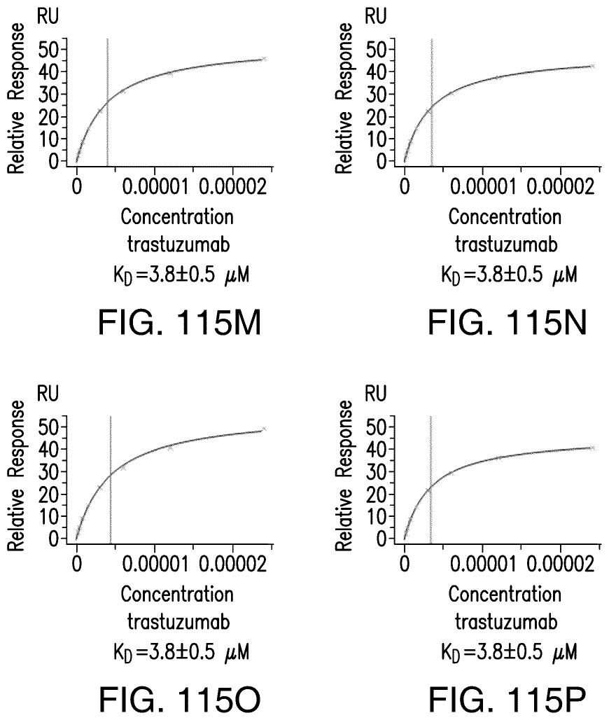

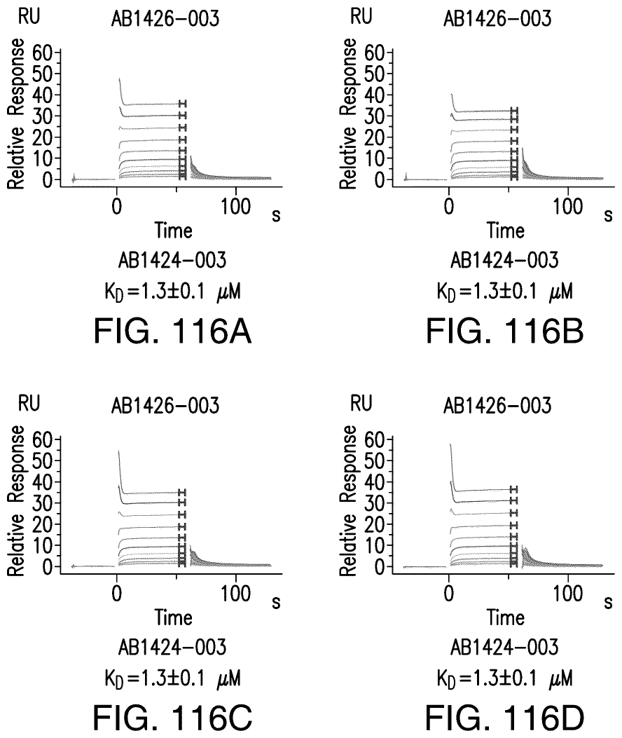

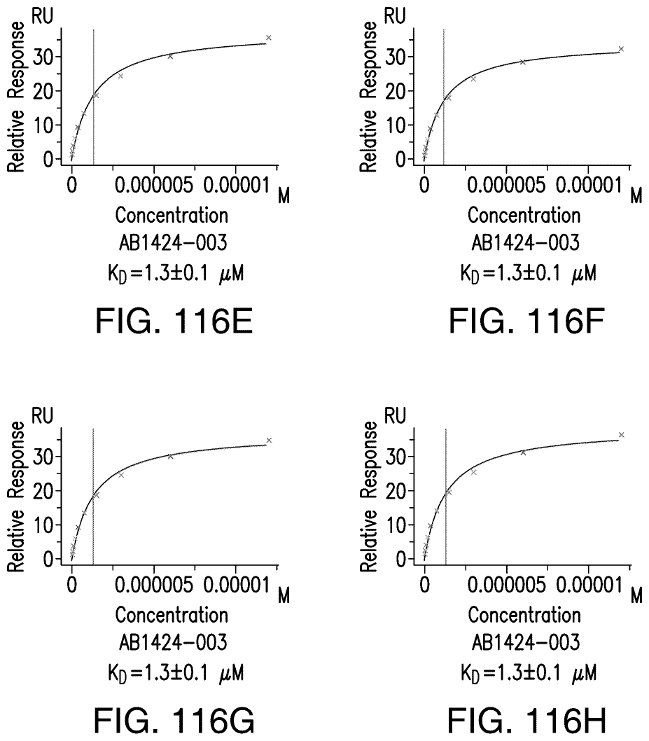

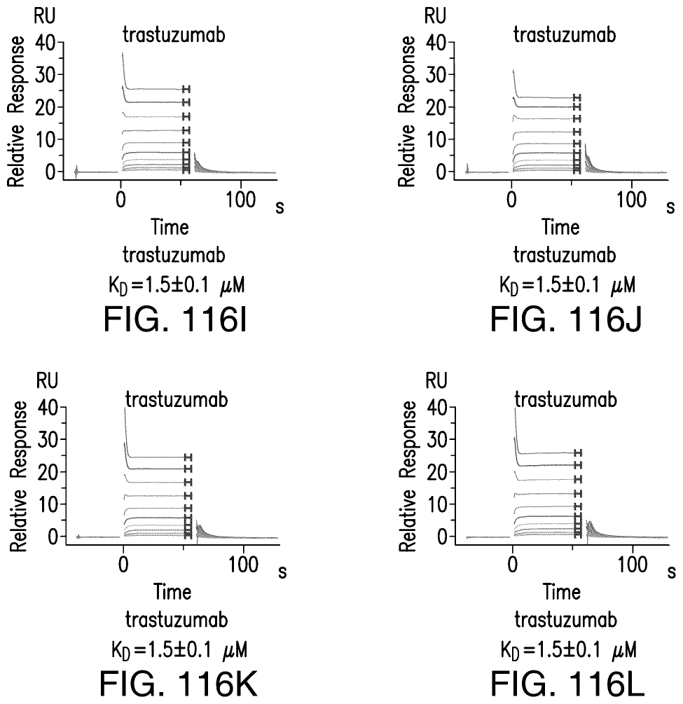

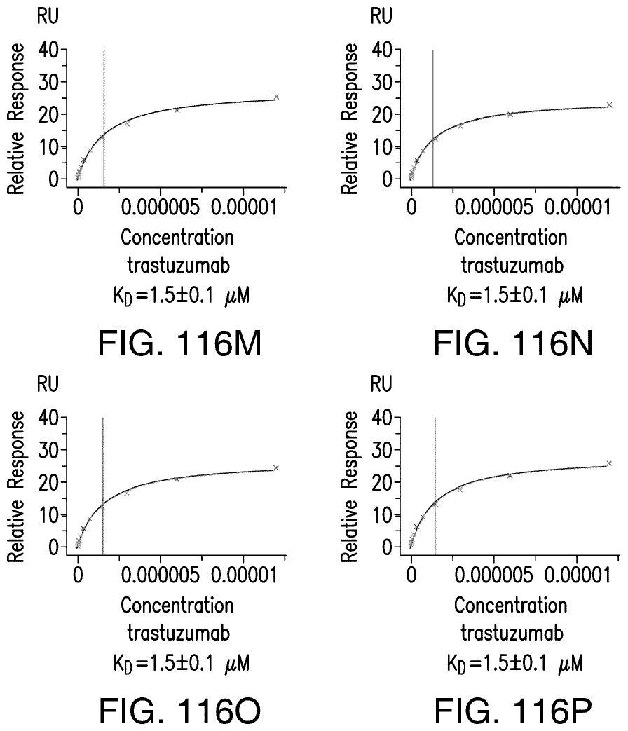

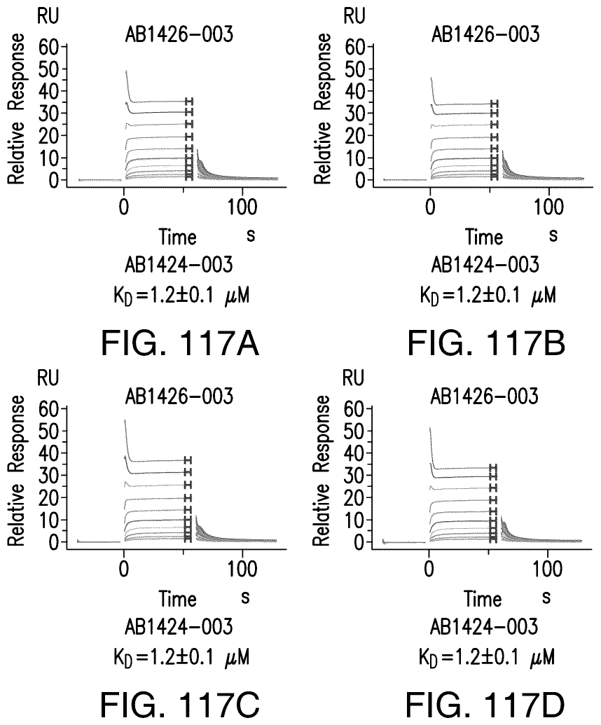

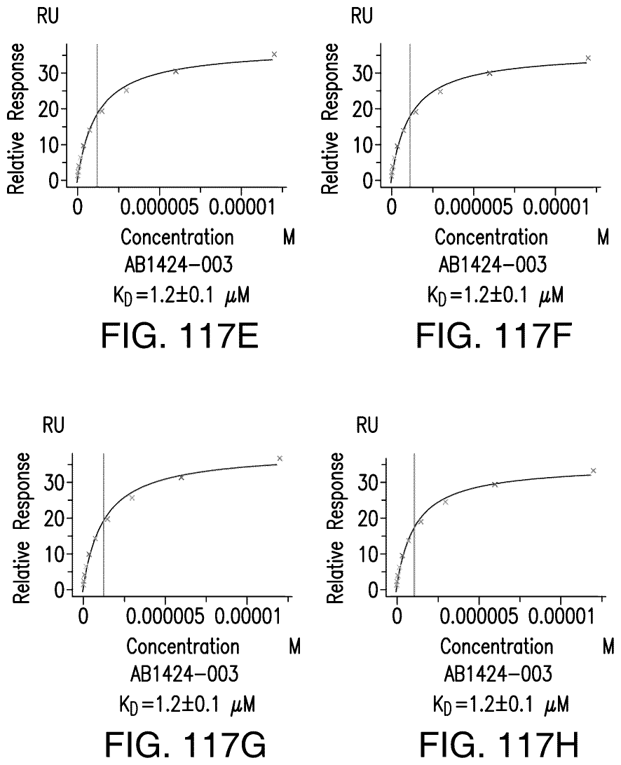

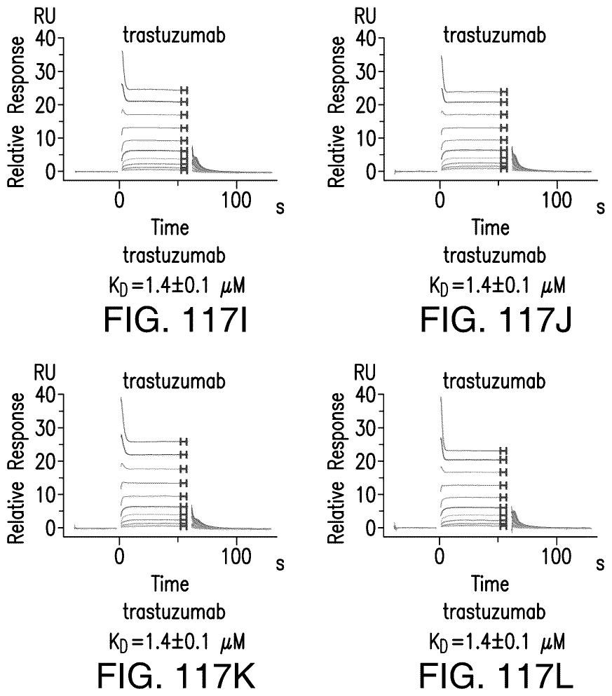

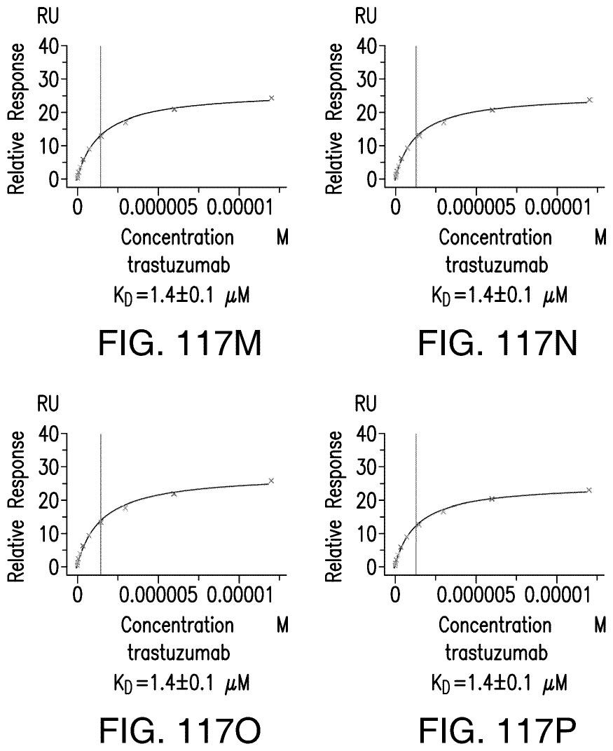

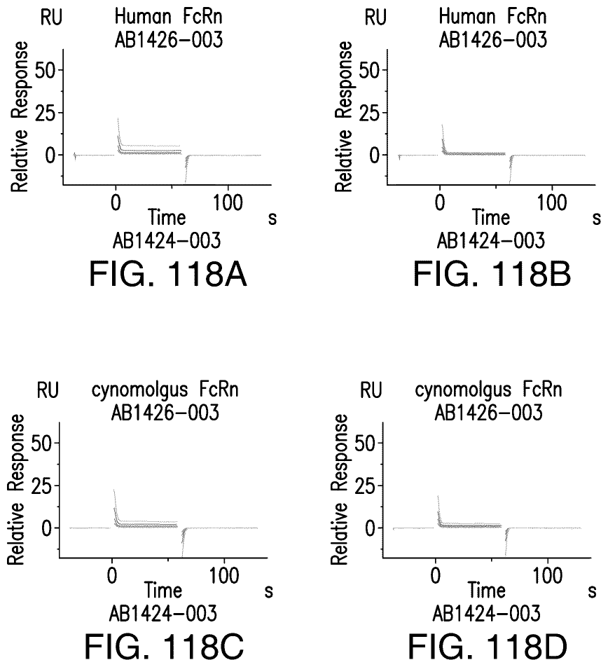

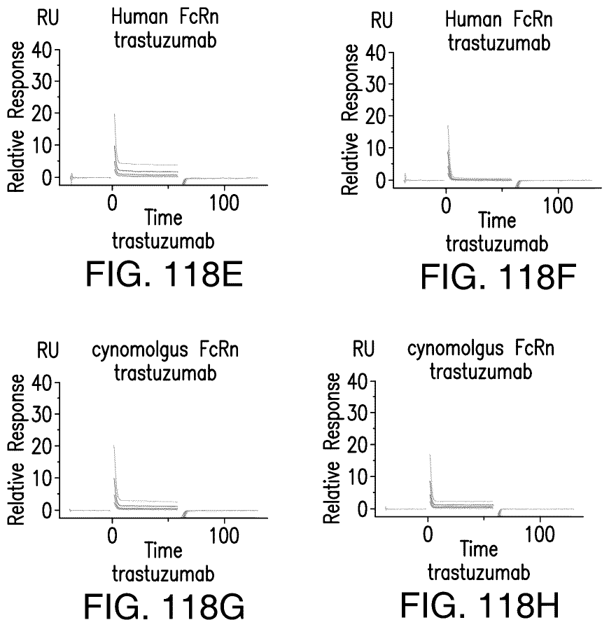

AB1424/AB1612 F4 TriNKET binds human and cynomolgus Fcγ receptors with affinities comparable to trastuzumab, a marketed IgG1 biologic that served an experimental control. Table 76 represents the summary of affinity values for the FcγRs tested. FIG. 111A-FIG. 111H, FIG. 112A-FIG. 112H, FIG. 113A-FIG. 113P, FIG. 114A-FIG. 114P, FIG. 115A-FIG. 115P, FIG. 116A-FIG. 116P, FIG. 117A-FIG. 117P, and FIG. 118A-FIG. 118H represent raw and fitted sensorgrams.

TABLE 76
Summary of affinities of AB1424/AB1612 F4 TriNKET and trastuzumab for human
and cynomolgus FcγR.

| | | | Human | | | | | | |
|---|---|---|---|---|---|---|---|---|---|
| | Human | Cyno | CD32a | | Human CD16a | | Human | Human | Cyno |
| | CD64 | CD64 | K_{D}, mM | | K_{D}, nM | | CD32b | CD16b | CD16 |
| Analyte | K_{D}, nM | K_{D}, nM | H131 | R131 | V158 | F158 | K_{D}, mM | K_{D}, mM | K_{D}, nM |
| AB1424/AB | 3.4 ± 0.3 | 0.7 ± 0.1 | 1.0 ± 0.1 | 1.4 ± 0.2 | 135.0 ± 10.0 | 1259.9 ± 98.5 | 6.1 ± 0.3 | 7.4 ± 1.0 | 232.1 ± 6.5 |
| 1612 F4 | | | | | | | | | |
| TRINKET | | | | | | | | | |
| trastuzumab | 2.7 ± 0.9 | 0.8 ± 0.1 | 1.3 ± 0.1 | 1.7 ± 0.2 | 65.6 ± 8.3 | 448.1 ± 20.1 | 7.2 ± 0.5 | 3.8 ± 0.5 | 73.7 ± 6.8 |

Binding of AB1424/AB1612 F4 TriNKET to human and cynomolgus FcRn was evaluated by SPR. Affinities of AB1424/AB1612 F4 TriNKET for human and cynomolgus FcRn were similar across the species and to those of trastuzumab, a marketed IgG1 biologic that served as an experimental control (Table 77). FIG. 116A-FIG. 116P and FIG. 117A-FIG. 117P represent steady-state fit and binding sensorgrams for human and cynomolgus FcRn binding, respectively, at pH 6.0. FIG. 118A-FIG. 118H demonstrate that, similar to IgG1 isotype control trastuzumab, AB1424/AB1612 F4 TriNKET lacked significant binding to human and cynomolgus FcRn at pH 7.4.

TABLE 77
Binding of AB1424/AB1612 F4 TriNKET and
trastuzumab to human and cynomolgus FcRn.

| | Human | Human | Cyno | Cyno |
|---|---|---|---|---|
| | FcRn, | FcRn, | FcRn, | FcRn, |
| | pH 6.0 | pH 7.4 | pH 6.0 | pH 7.4 |
| Analyte | K_{D}, μM | K_{D}, μM | K_{D}, μM | K_{D}, μM |
| AB1424/ | 1.3 ± 0.0 | No | 1.2 ± 0.0 | No |
| AB1612 | | quantifiable | | quantifiable |
| F4 TriNKET | | binding | | binding |
| trastuzumab | 1.5 ± 0.1 | No | 1.4 ± 0.1 | No |
| | | quantifiable | | quantifiable |
| | | binding | | binding |

### Co-Engagement of Antigen-Binding Sites

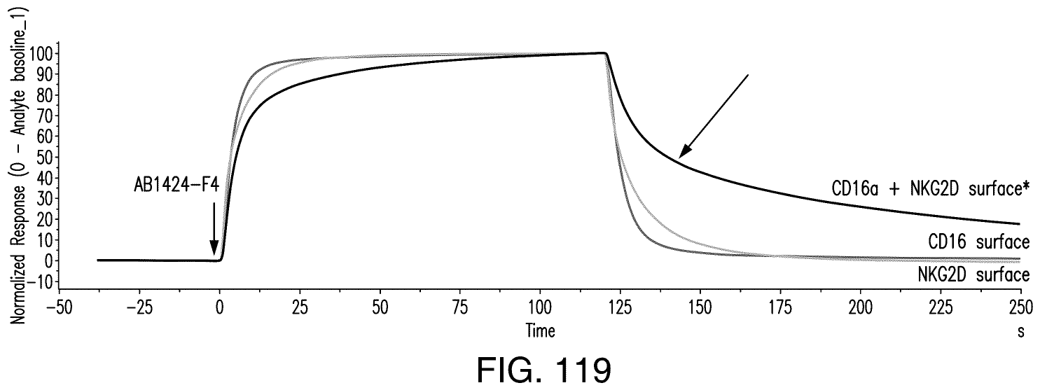

To demonstrate synergy of co-engagement of human CD16a and human NKG2D binding, an SPR experiment was performed where binding of AB1424/AB1612 F4 TriNKET to NKG2D and CD16a separately compared to a mixed NKG2D-CD16a Biacore chip surface. The affinity of AB1424/AB1612 F4 TriNKET for human NKG2D and human CD16a were both low, however, binding to both targets simultaneously resulted in an avidity effect that manifested as a slower off-rate. The data demonstrated that AB1424/AB1612 F4 TriNKET can avidly engage CD16a and NKG2D (FIG. 119).

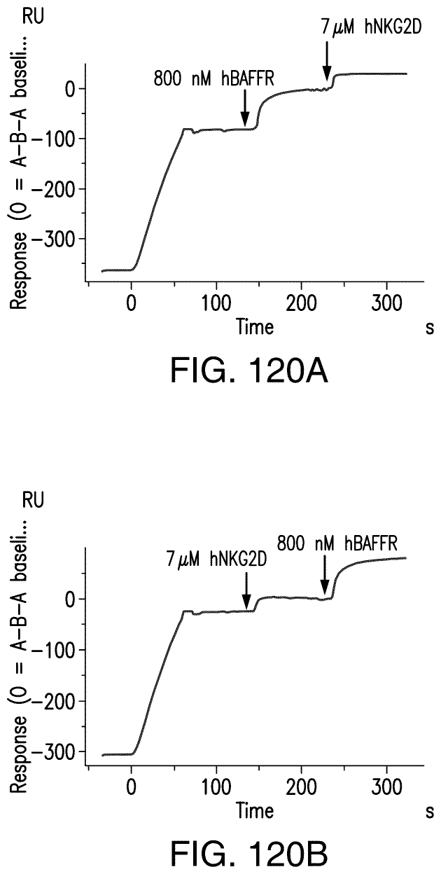

To determine if binding of AB1424/AB1612 F4 TriNKET to one target interferes with its binding to the other target, BAFF-R and NKG2D were sequentially injected over AB1424/AB1612 F4 TriNKET captured on an anti-hFc IgG SPR chip (FIG. 120A). Target binding sensorgrams demonstrated that the occupancy status of the BAFF-R binding arm does not interfere with NKG2D binding (FIG. 120A). Likewise, the data demonstrated that occupancy of the NKG2D binding arm does not prohibit BAFF-R association (FIG. 120B). Similarity in shapes of the respective sensorgram segments depicting binding of each target to free AB1424/AB1612 F4 TriNKET and AB1424/AB1612 F4 TriNKET that has been saturated with the other target suggested that the kinetic parameters were not meaningfully affected by the target occupancy status of the AB1424/AB1612 F4 TriNKET. For instance, the shape of the BAFF-R binding segment of the sensorgram is similar in both panels. Saturating concentrations of the NKG2D had to be maintained throughout the entire experiment represented in the lower panel due to the fast dissociation rate of this target. Additionally, the lack of any impact on relative stoichiometry of each target binding (when compared to binding to unoccupied AB1424/AB1612 F4 TriNKET) signifies full independence of NKG2D and BAFF-R binding sites on AB1424/AB1612 F4 TriNKET (Table 78).

TABLE 78
Relative binding stoichiometries of AB1424/AB1612 F4
TriNKET for BAFF-R and NKG2D.

| | BAFF-R | NKG2D |
|---|---|---|
| | relative binding | relative binding |
| Experimental setup | stoichiometry | stoichiometry |
| Target Bound to | 1.00 | 1.00 |
| AB1424/AB1612 F4 | | |
| TriNKET unoccupied with | | |
| another target (injected first) | | |
| Target Bound to | 0.97 ± 0.03 | 1.24 ± 0.22 |
| AB1424/AB1612 F4 | | |
| TriNKET saturated with | | |
| another target (injected | | |
| second) | | |

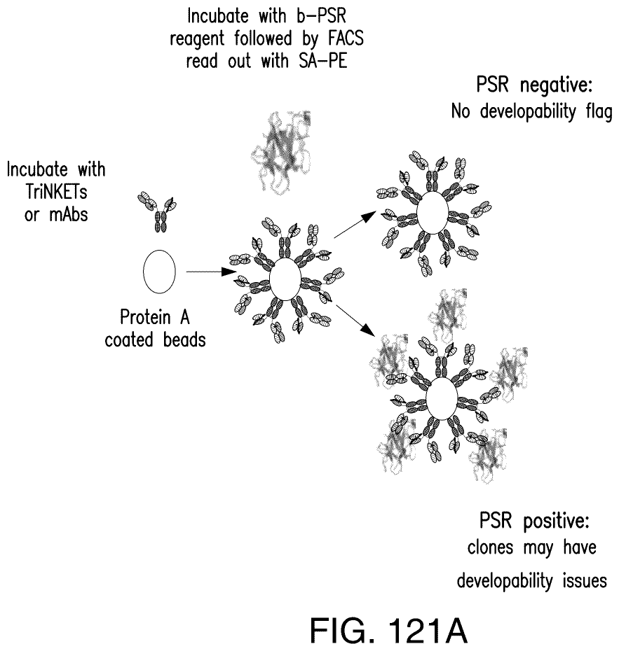

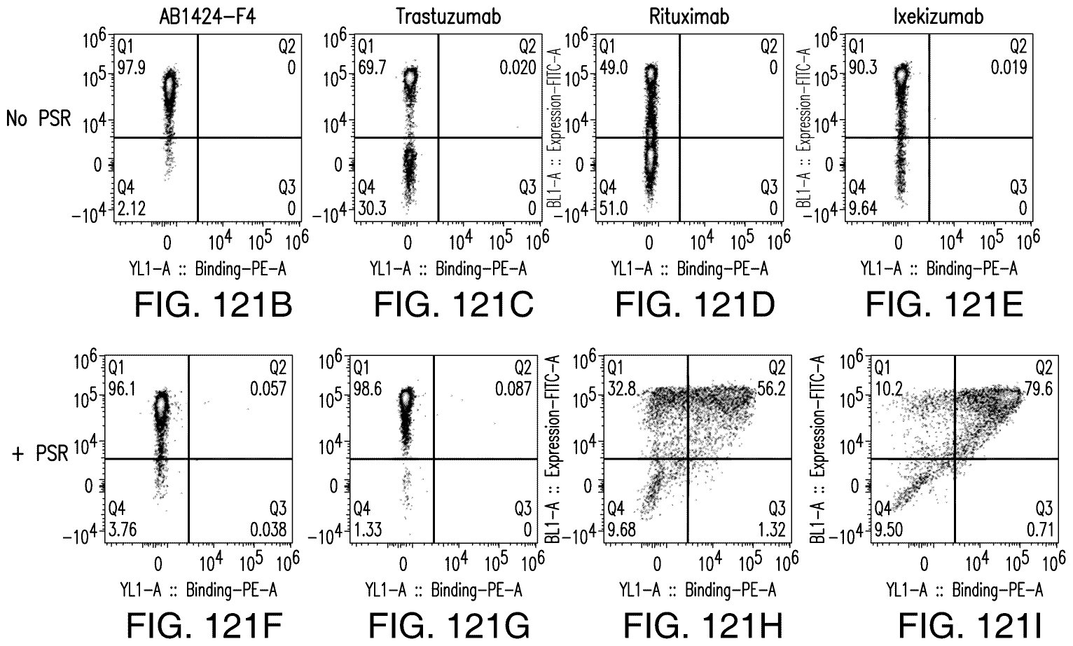

To assess the specificity of AB1424/AB1612 F4 TriNKET, a flow cytometry based PSR assay was performed as described in Example 4 above. AB1424/AB1612 F4 TriNKET was negative for binding to PSR and was most comparable to the PSR of the negative control, trastuzumab (FIG. 121A-FIG. 121I). These results indicate that AB1424/AB1612 F4 TriNKET does not exhibit reactivity with non-specific proteins.

### Potency of AB1424/AB1612 F4 TriNKET

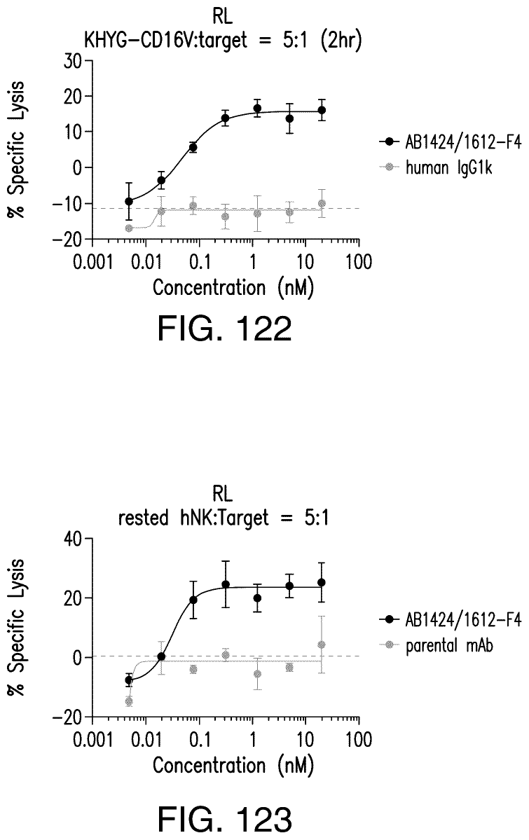

Potency of AB1424/AB1612 F4 TriNKET was assessed by its ability to stimulate KHYG-1-CD16aV mediated cytolysis of BAFF-R⁺ RL cells (FIG. 122). AB1424/AB1612 F4 TriNKET is highly potent in driving the lysis of BAFF-R+ RL cells, exhibiting sub-nanomolar potency and effective maximum cell killing (Table 79).

TABLE 79
Potency of AB1424/AB1612 F4 TriNKET in the presence
of KHYG-1-CD16aV and RL cells.

| Test article | EC₅₀ (nM) | Max killing (%) |
|---|---|---|
| AB1424/AB1612 F4 | 0.05 | 16 |
| TriNKET | | |

Potency of AB1424/AB1612 F4 TriNKET in the primary NK cell-mediated lysis of BAFF-R⁺ tumor cell line RL was further compared to the parental mAb (FIG. 123). Parental mAb elicited low or no detectable cytolysis of BAFF-R⁺ cell line RL. AB1424/AB1612 F4 TriNKET demonstrated a sub-nanomolar EC₅₀, efficient maximum killing, and exceeded the potency of parental mAb (FIG. 123 and Table 80).

TABLE 80
Potency of AB1424/AB1612 F4 TriNKET in the presence
of primary NK and BAFF-R⁺ cells.

| | AB1424/AB1612 F4 TriNKET | | parental mAb | |
|---|---|---|---|---|
| | EC₅₀ | Max | EC₅₀ | Max |
| Cell line | (nM) | killing (%) | (nM) | killing (%) |
| RL | 0.03 ± 0.00 | 28 ± 15 | ND | ND |

ND = Not determined

### Developability of AB1424/AB1612 F4 TriNKET

Developability of AB1424/AB1612 F4 TriNKET was assessed by applying a series of stresses: thermal stress (40° C., 4 weeks), low pH stress (pH 5, 40° C., 2 weeks), high pH stress (pH 8, 40° C., 2 weeks), oxidation stress (0.02% hydrogen peroxide, 25° C., 24 h), agitation, freeze/thaw, low pH hold.

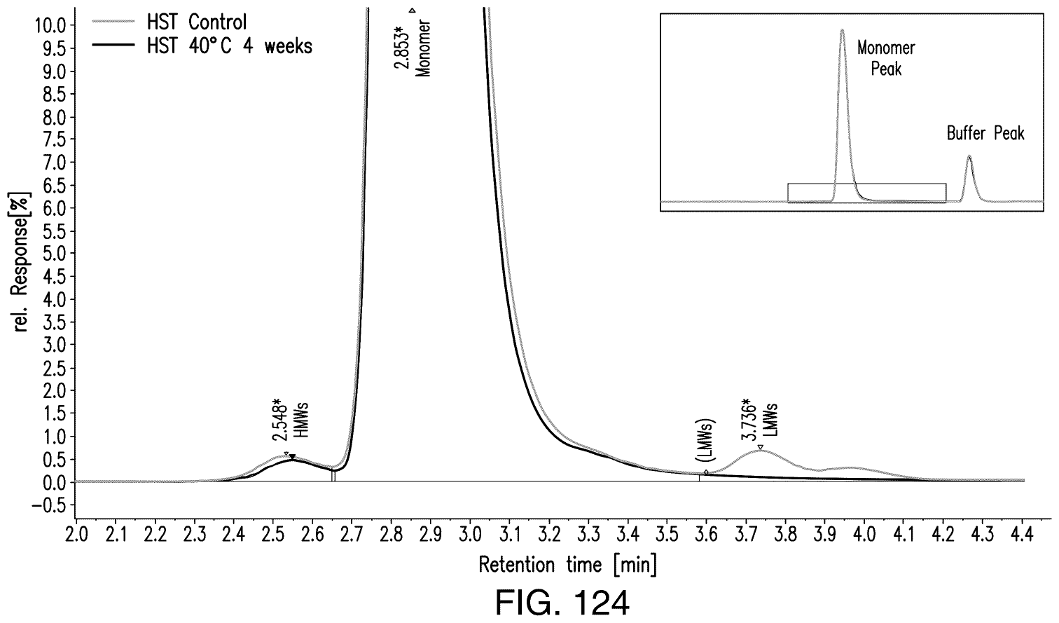

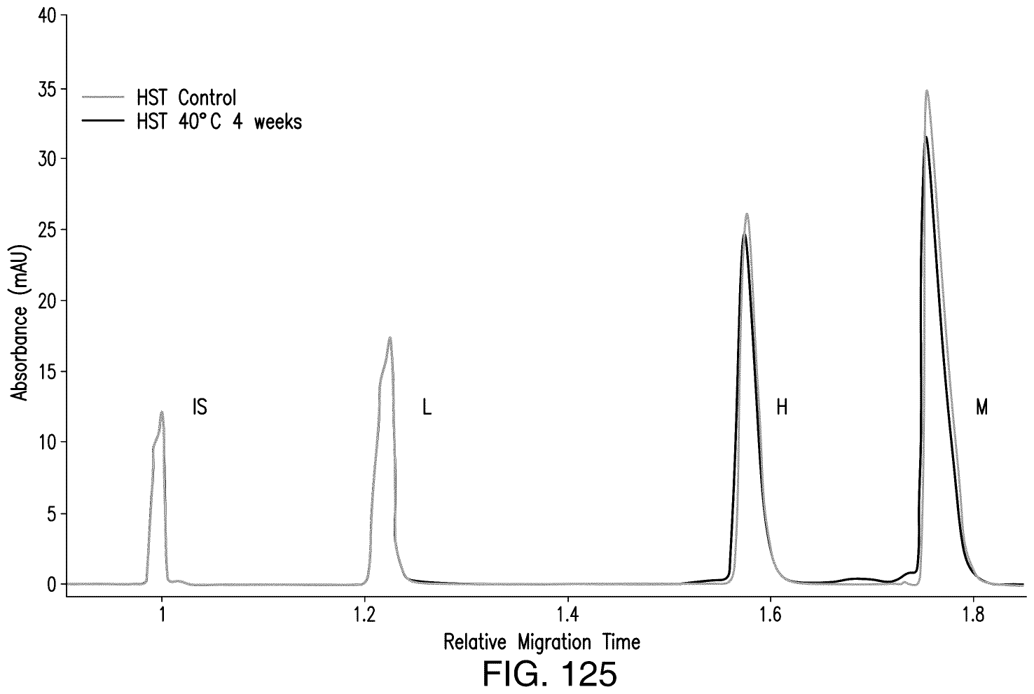

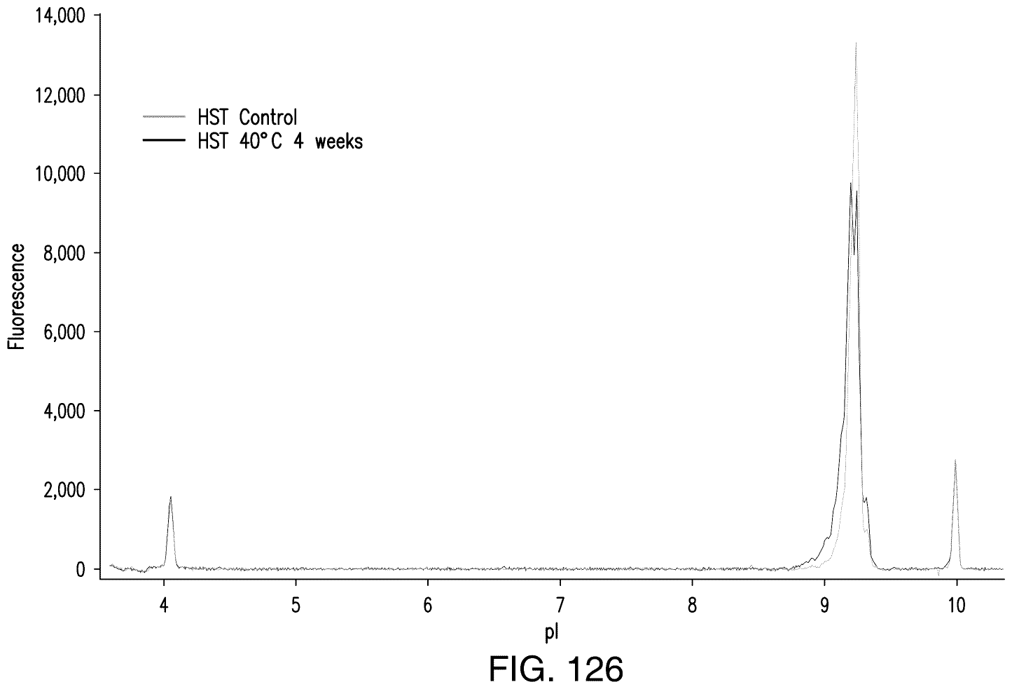

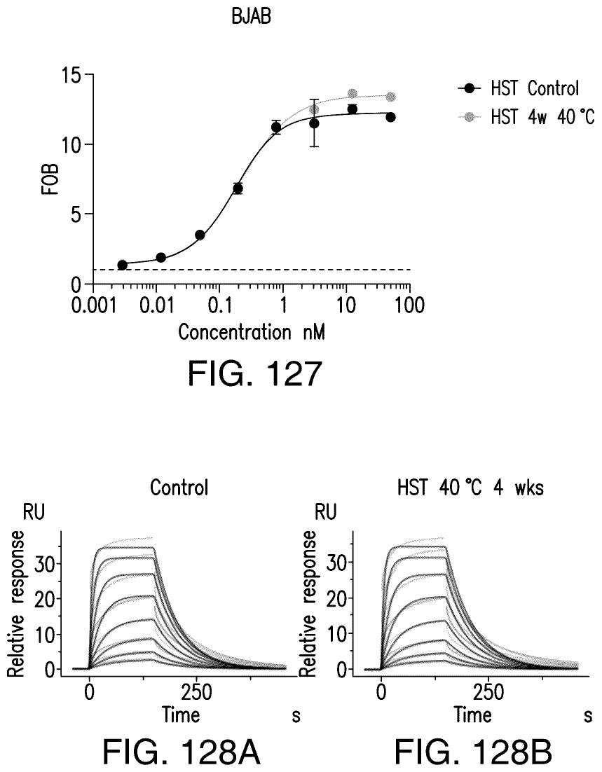

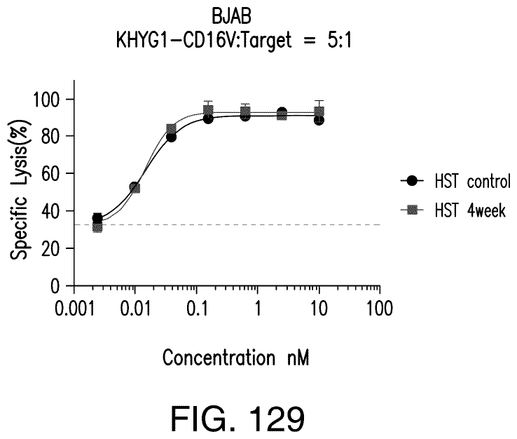

AB1424/AB1612 F4 TriNKET demonstrated high stability after 4 weeks of incubation in HST as described above, pH 6.0, at 40° C. Very little aggregation (+0.1%) and minimal loss of monomer (1.0%) was observed by SEC (FIG. 124 and Table 81). A 2.6% loss in purity was detected by R CE-SDS (FIG. 125 and Table 82). The charge profile monitored by cIEF demonstrated an acidic shift from 52.5% main in the control to 29.9% main after 4 weeks (FIG. 126 and Table 83). This loss in main peak is typical of proteins incubated at elevated temperatures. Additionally, there was no meaningful difference observed in AB1424/AB1612 F4 TriNKET binding to human BAFF-R⁺ cells or the kinetics and affinity to human CD16aV between control and stressed samples (FIG. 127, Table 84, FIG. 128A, FIG. 128B, and Table 85). No difference in potency between the control and stressed samples was detected (FIG. 129 and Table 86).

TABLE 81
Summary of AB1424/AB1612 F4 TriNKET monomer, HMWS
and LMWS after incubation at 40° C. in HST, pH 6.0.

| | Monomer | HMWS | LMWS |
|---|---|---|---|
| Test Article | (%) | (%) | (%) |
| AB1424/AB1612 F4 TriNKET | 99.5 | 0.5 | 0.0 |
| HST, pH 6.0 control* | | | |
| AB1424/AB1612 F4 TriNKET, | 98.5 | 0.6 | 0.9 |
| HST, pH 6.0, 40° C., 4 wks | | | |

*HST, pH 6.0 control sample stored at −80° C. after staging, prior to analysis.

TABLE 82
Summary of AB1424/AB1612 F4 TriNKET R CE-SDS
purity after 4 weeks at 40° C. in HST, pH 6.0.

| Test Article | R CE-SDS Purity (%) |
|---|---|
| AB1424/AB1612 F4 TriNKET, | 99.8 |
| HST, pH 6.0 control | |
| AB1424/AB1612 F4 TriNKET, | 97.2 |
| HST, pH 6.0, 40° C., 4 wks | |

TABLE 83
Summary of acidic, main, and basic species in AB1424/
AB1612 F4 TriNKET after incubation at 40° C. in HST, pH 6.0.

| Test Article | Acidic (%) | Main (%) | Basic (%) |
|---|---|---|---|
| AB1424/AB1612 F4 TriNKET, | 44.1 | 52.5 | 3.5 |
| HST, pH 6.0 control | | | |
| AB1424/AB1612 F4 TriNKET, | 64.7 | 29.9 | 5.2 |
| HST, pH 6.0, 40° C., 4 wks | | | |

TABLE 84
Binding of AB1424/AB1612 F4 TriNKET to hBAFF-R⁺
cells after 4 weeks at 40° C. in HST, pH 6.0.

| | | Binding to BAFF-R⁺ isogenic |
|---|---|---|
| Test Article | Target | CHO cells EC₅₀, nM |
| AB1424/AB1612 F4 TriNKET, | hBAFF-R | 0.18 |
| HST, pH 6.0 control | | |
| AB1424/AB1612 F4 TriNKET, | hBAFF-R | 0.22 |
| HST, pH 6.0, 40° C., 4 wks | | |

Results are an average of n = 3 replicates

TABLE 85
Kinetic parameters and binding affinity of AB1424/AB1612 F4
TriNKET for hCD16a after 4 weeks at 40° C. in HST, pH 6.0.

| Test Article | Target | kₐ (M⁻¹s⁻¹) | k_{d} (s⁻¹) | K_{D} (nM) |
|---|---|---|---|---|
| AB1424/AB1612 F4 | hCD16aV | (1.2 ± 0.1) × | (1.8 ± 0.0) × | 149.8 ± 4.3 |
| TriNKET, HST, | | 10⁵ | 10⁻² | |
| pH 6.0 control | | | | |
| AB1424/AB1612 F4 | hCD16aV | (1.1 ± 0.1) × | (1.6 ± 0.0) × | 145.5 ± 16.6 |
| TriNKET, HST, | | 10⁵ | 10⁻² | |
| pH 6.0, 40° C., 4 wks | | | | |

Results are an average of n = 3 replicates

TABLE 86
Summary of AB1424/AB1612 F4 TriNKET EC₅₀ and
maximum lysis after 4 weeks in HST, pH 6.0.

| Test Article | EC₅₀ (nM) | Max killing (%) |
|---|---|---|
| AB1424/AB1612 F4 TriNKET, | 0.01 | 91 |
| HST, pH 6.0 control | | |
| AB1424/AB1612 F4 TriNKET, | 0.01 | 93 |
| HST, pH 6.0, 40° C., 4 wks | | |

### Chemical Stability of AB1424/AB1612 F4 TriNKET

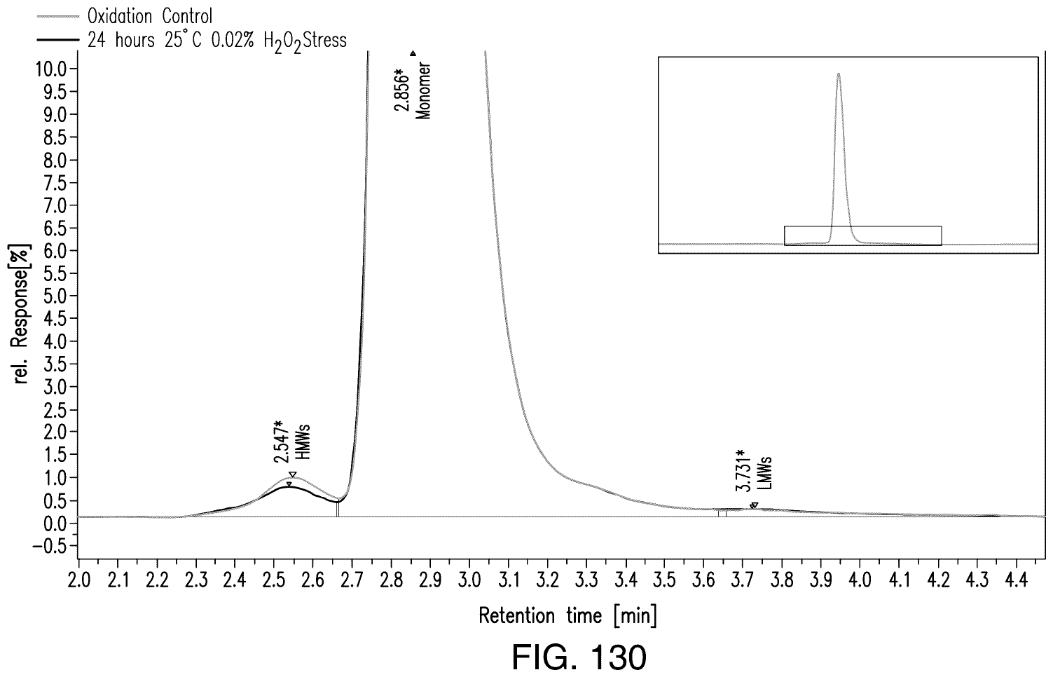

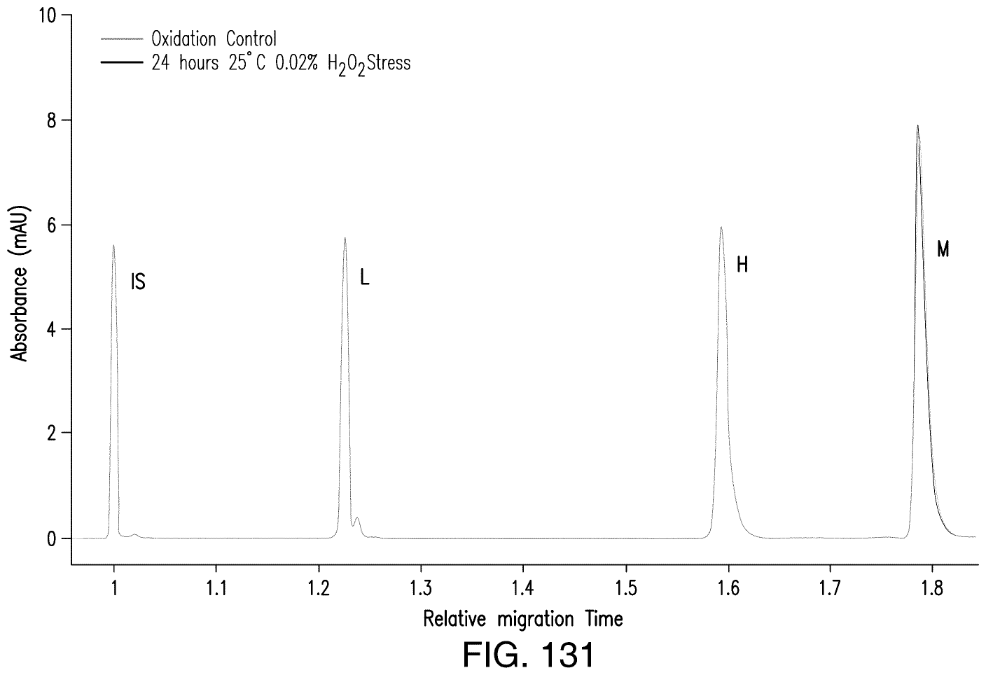

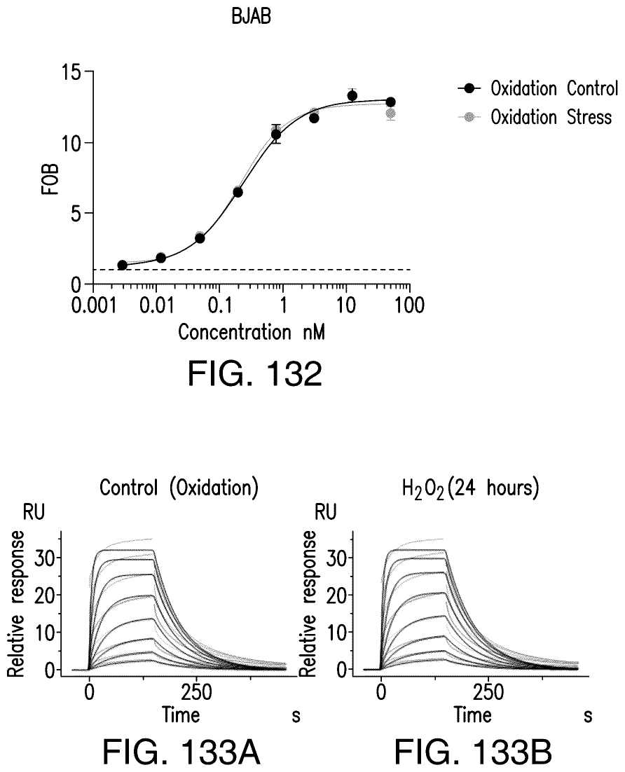

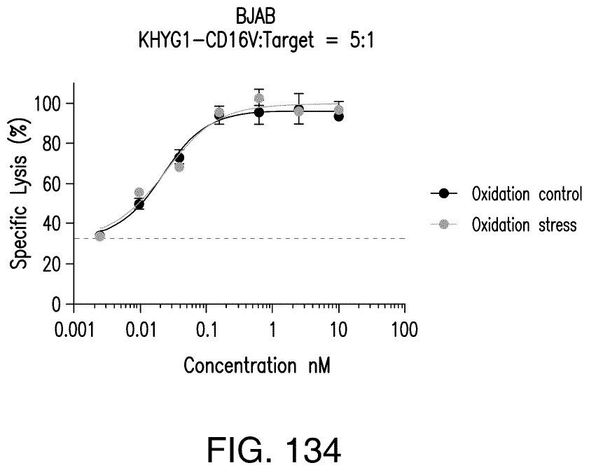

To assess stability of AB1424/AB1612 F4 TriNKET under oxidative stress, AB1424/AB1612 F4 TriNKET was incubated with 0.02% hydrogen peroxide for 24 hours at 25° C. in PBS. No aggregation or loss of monomer was observed by SEC (FIG. 130 and Table 87). No meaningful increase in fragmentation was detected by R CE-SDS (FIG. 131 and Table 88). Additionally, there was no meaningful difference observed in binding to hBAFF-R cells or in the kinetics and affinity for hCD16a between control and stressed samples (FIG. 132, Table 89, FIG. 133A, FIG. 133B, and Table 90). Finally, the potency of AB1424/AB1612 F4 TriNKET in the KHYG-1-CD16aV cytotoxicity assay was unchanged after oxidative stress (FIG. 134 and Table 91).

TABLE 87
Summary of AB1424/AB1612 F4 TriNKET monomer,
HMWS, and LMWS after forced oxidation.

| Test Article | Monomer (%) | HMWS (%) | LMWS (%) |
|---|---|---|---|
| AB1424/AB1612 F4 | 98.6 | 1.0 | 0.4 |
| TriNKET, oxidation | | | |
| control* | | | |
| AB1424/AB1612 F4 | 98.8 | 0.8 | 0.4 |
| TriNKET, 0.02% H₂O₂, | | | |
| 24 hrs | | | |

*oxidation control sample (mock diluted with PBS instead of H₂O₂) stored at −80° C. after staging, prior to analysis.

TABLE 88
Summary of AB1424/AB1612 F4 TriNKET R CE-SDS
purity after forced oxidation.

| Test Article | R CE-SDS purity (%) |
|---|---|
| AB1424/AB1612 F4 TriNKET, oxidation | 100.0 |
| control | |
| AB1424/AB1612 F4 TriNKET 0.02% H₂O₂, | 100.0 |
| 24 hrs | |

TABLE 89
Binding of AB1424/AB1612 F4 TriNKET to
hBAFF-R⁺ cells after forced oxidation.

| | | Binding to BAFF-R |
|---|---|---|
| Test Article | Target | Expressing Cells EC₅₀, nM |
| AB1424/AB1612 F4 TriNKET, | hBAFF-R | 0.24 |
| oxidation control | | |
| AB1424/AB1612 F4 TriNKET, | hBAFF-R | 0.21 |
| 0.02% H₂O₂, 24 hrs | | |

TABLE 90
Kinetic parameters and binding affinity of AB1424/AB1612 F4
TriNKET for hCD16a after forced oxidation.

| Test Article | Target | kₐ (M⁻¹s⁻¹) | k_{d} (s⁻¹) | K_{D} (nM) |
|---|---|---|---|---|
| AB1424/AB1612 F4 | hCD16aV | (1.1 ± 0.0) × 10⁵ | (1.6 ± 0.0) × 10⁻² | 137.1 ± 3.2 |
| TriNKET, oxidation | | | | |
| control | | | | |
| AB1424/AB1612 F4 | hCD16aV | (1.2 ± 0.0) × 10⁵ | (1.5 ± 0.0) × 10⁻² | 131.1 ± 4.0 |
| TriNKET, 0.02% | | | | |
| H₂O₂, 24 hrs | | | | |

Results are an average of n = 3 replicates

TABLE 91
Summary of AB1424/AB1612 F4 TriNKET EC₅₀ and
maximum lysis after forced oxidation.

| Test article | EC₅₀ (nM) | Max killing (%) |
|---|---|---|
| AB1424/AB1612 F4 | 0.02 | 96 |
| TriNKET, oxidation control | | |
| AB1424/AB1612 F4 | 0.02 | 100 |
| TriNKET, 0.02% H₂O₂, 24 | | |
| hrs | | |

### Long Term pH 5 Stress

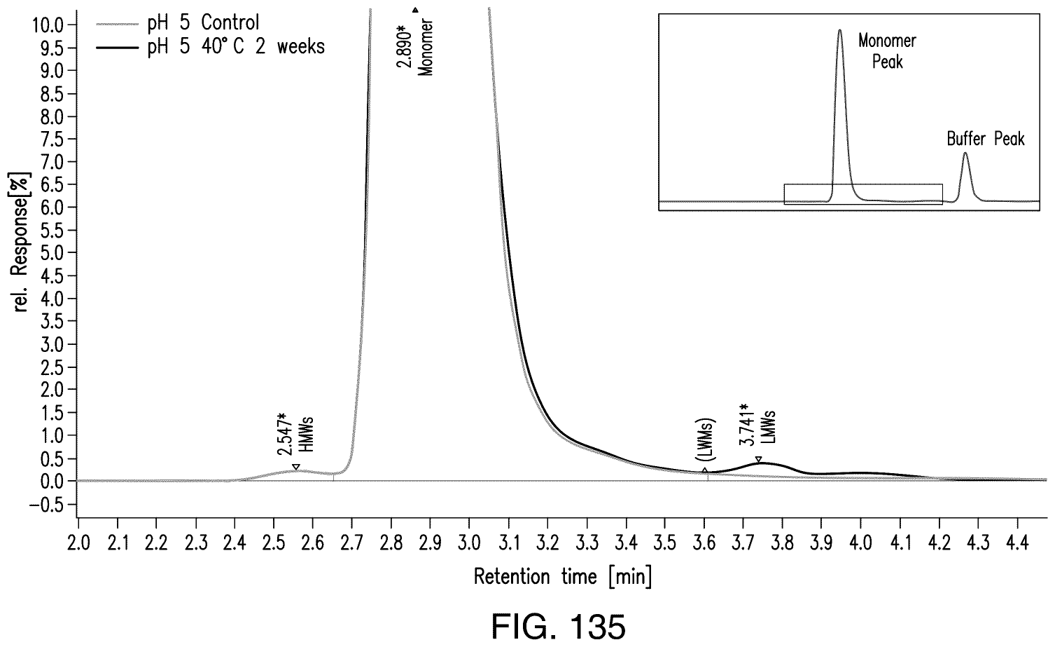

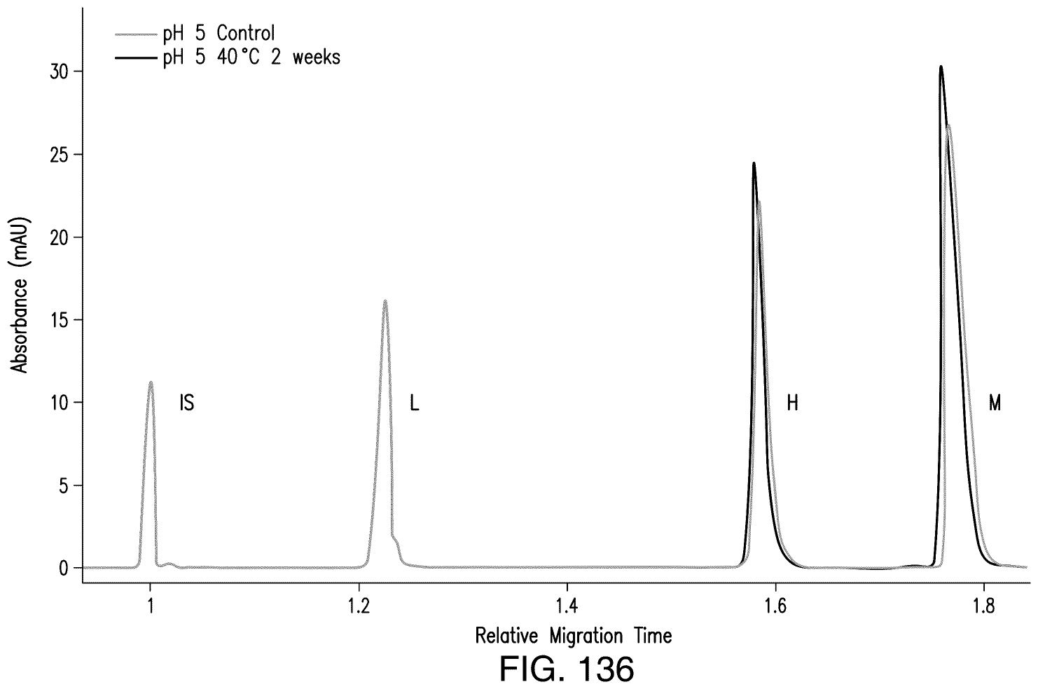

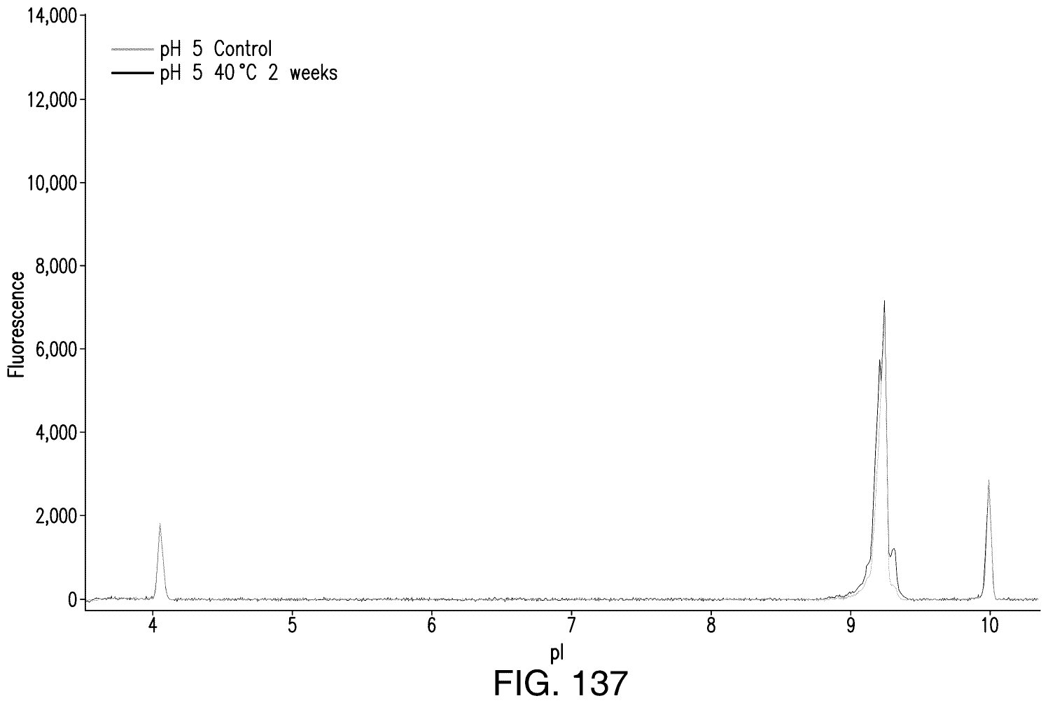

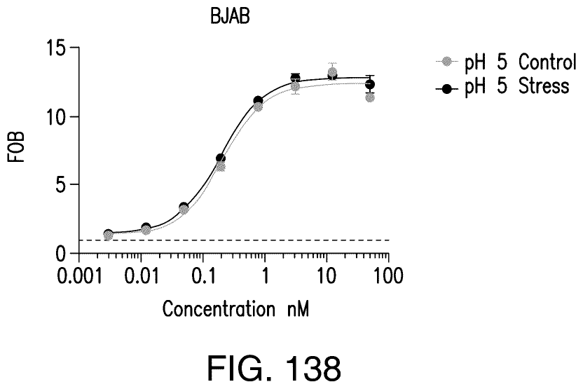

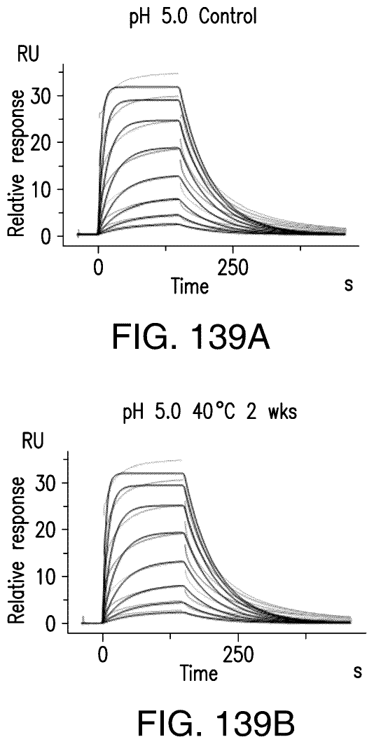

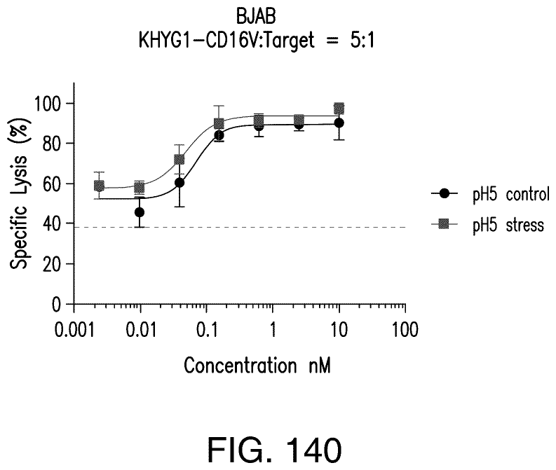

The chemical stability of AB1424/AB1612 F4 TriNKET was assessed by long term incubation at low pH (20 mM sodium acetate, pH 5.0, 40° C., 2 weeks). No aggregation and minimal loss of monomer (0.6%) were observed by SEC (FIG. 135 and Table 92). No meaningful increase in fragmentation was detected by reduced CE-SDS (FIG. 136 and Table 93). After long term pH 5 stress, the charge profile monitored by cIEF demonstrated an acidic shift from 52.9% main in the control to 41.2% main in the stressed sample (FIG. 137 and Table 94). Long term pH 5 stress had no significant effect on binding to hBAFF-R⁺ cells or the kinetics and affinity for hCD16aV (FIG. 138, Table 95, FIG. 139A, FIG. 139B, and Table 96). Additionally, no significant difference in potency in KHYG-1-CD16aV mediated cytotoxicity assay between stressed and control sample was observed (FIG. 140 and Table 97). Based on these results, it was concluded that AB1424/AB1612 F4 TriNKET is resistant to aggregation and fragmentation due to low pH stress.

TABLE 92
Summary of AB1424/AB1612 F4 TriINKET monomer,
HMWS, and LMWS after long term low pH stress.

| Test Article (%) | Monomer (%) | HMWS (%) | LMWS (%) |
|---|---|---|---|
| AB1424/AB1612 F4 | 99.7 | 0.3 | 0.0 |
| TriNKET pH 5 control | | | |
| AB1424/AB1612 F4 | 99.1 | 0.2 | 0.7 |
| TriNKET, pH 5, 40° C., 2 wks | | | |

TABLE 93
Summary of AB1424/AB 1612 F4 TriNKET R CE-SDS
purity after long term low pH stress.

| Test Article | R CE-SDS purity (%) |
|---|---|
| AB1424/AB1612 F4 TriNKET, pH 5 control* | 99.8 |
| AB1424/AB1612 F4 TriNKET, pH 5, 40° C., 2 | 99.6 |
| wks | |

*pH 5 control sample stored at −80° C. after staging, prior to analysis.

TABLE 94
Summary of acidic, main, and basic species in AB1424/AB1612
F4 TriNKET after long term low pH stress.

| Test Article | Acidic (%) | Main (%) | Basic (%) |
|---|---|---|---|
| AB1424/AB1612 F4 | 44.2 | 52.9 | 2.9 |
| TriNKET, pH 5 control | | | |
| AB1424/AB1612 F4 | 50.4 | 41.2 | 8.4 |
| TriNKET, pH 5, 40° C., 2 | | | |
| wks | | | |

TABLE 95
Binding of AB1424/AB1612 F4 TriNKET to hBAFF-R⁺
cells after long term low pH stress,

| | | Binding to BAFF-R |
|---|---|---|
| | | Expressing Cells |
| Test Article | Target | EC₅₀, nM |
| AB1424/AB1612 F4 TriNKET, pH | hBAFF-R | 0.22 |
| 5 control | | |
| AB1424/AB1612 F4 TriNKET, pH | hBAFF-R | 0.19 |
| 5, 40° C., 2 wks | | |

TABLE 96
Kinetic parameters and binding affinity of AB1424/AB1612 F4 TriNKET for
hCD16a after long term low pH stress.

| Test Article | Target | kₐ (M⁻¹s⁻¹) | k_{d} (s⁻¹) | K_{D} (nM) |
|---|---|---|---|---|
| AB1424/AB1612 F4 | hCD16aV | (1.1 ± 0.0) × 10⁵ | (1.7 ± 0.0) × 10⁻² | 150.5 ± 11.9 |
| TriNKET, pH 5 control | | | | |
| AB1424/AB1612 F4 | hCD16aV | (1.1 ± 0.0) × 10⁵ | (1.6 ± 0.0) × 10⁻² | 152.6 ± 3.9 |
| TriNKET, pH 5, 40° C., 2 | | | | |
| wks | | | | |

Results are an average of n = 3 replicates

TABLE 97
Summary of AB1424/AB1612 F4 TriNKET EC₅₀ and
maximum lysis after long term low pH exposure.

| Test Article | EC₅₀ (nM) | Max killing (%) |
|---|---|---|
| AB1424/AB1612 F4 | 0.07 | 89 |
| TriNKET, pH 5 control | | |
| AB1424/AB1612 F4 | 0.05 | 93 |
| TriNKET, pH 5, 40° C., 2 wks | | |

### Long Term pH 8 Stress

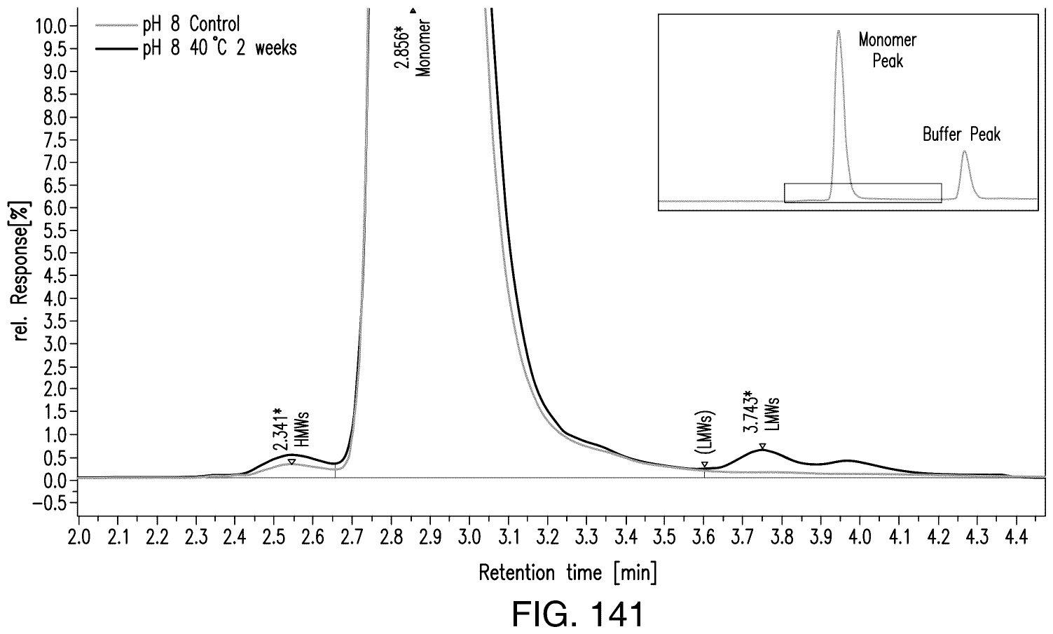

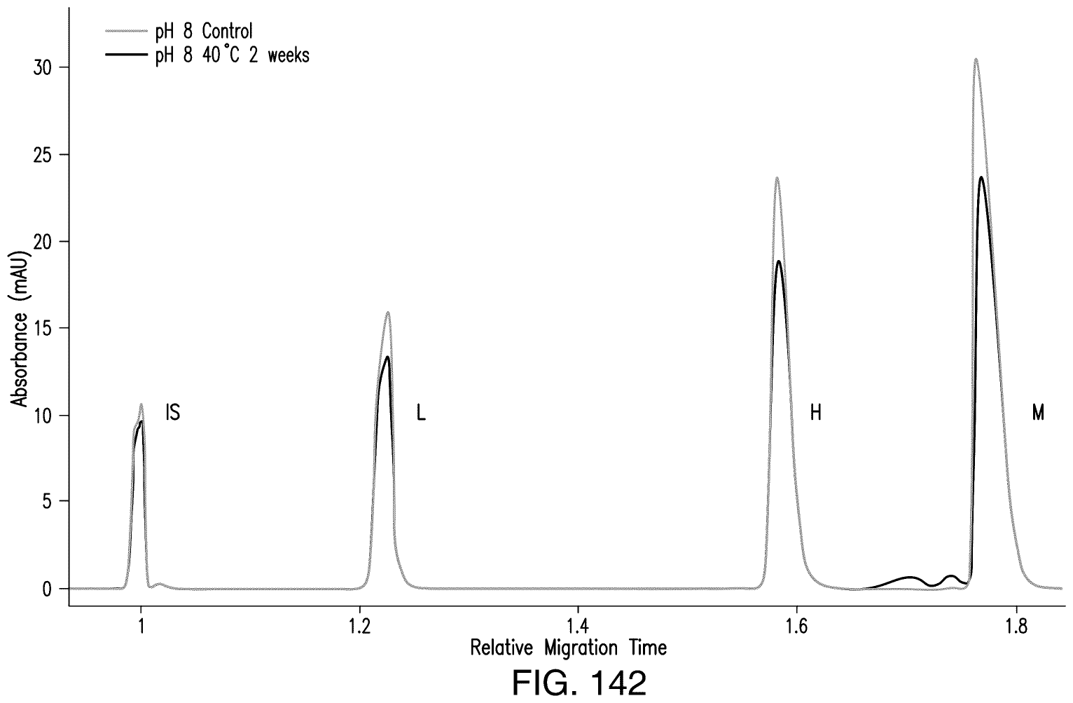

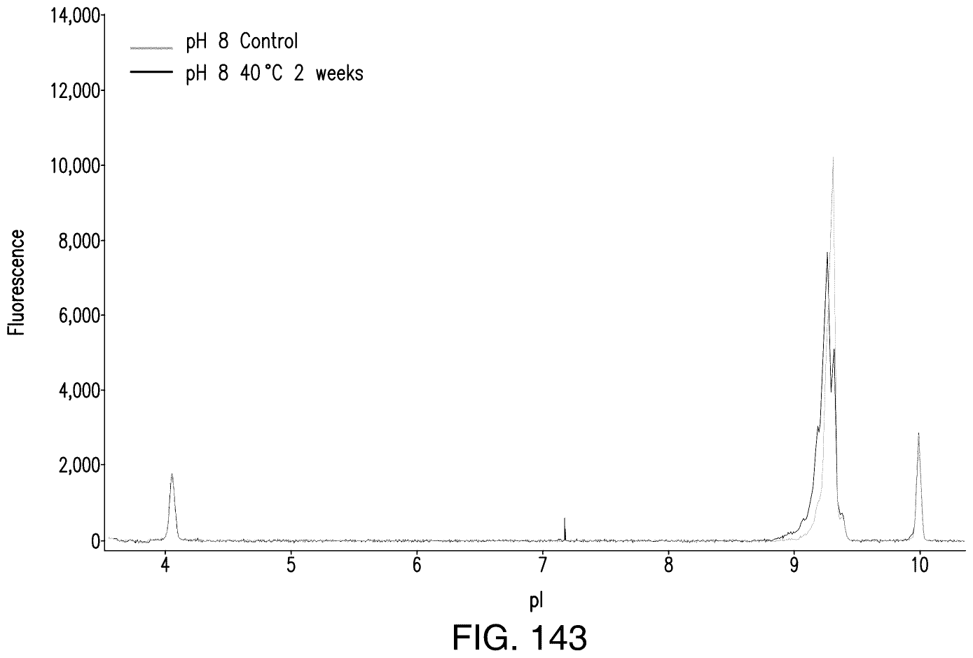

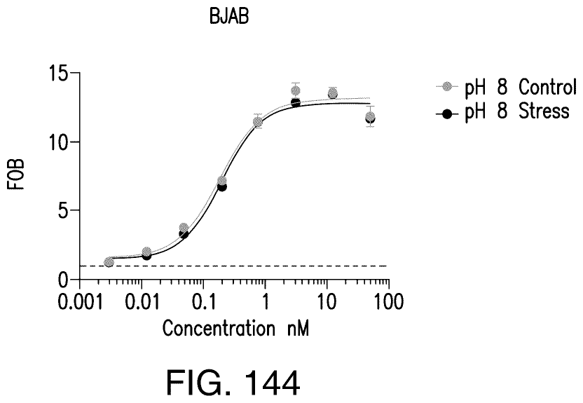

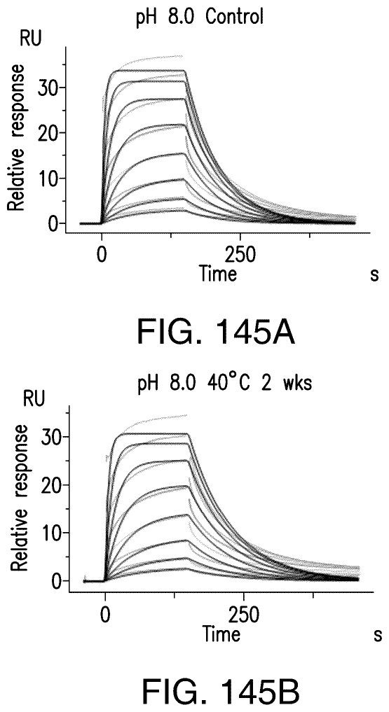

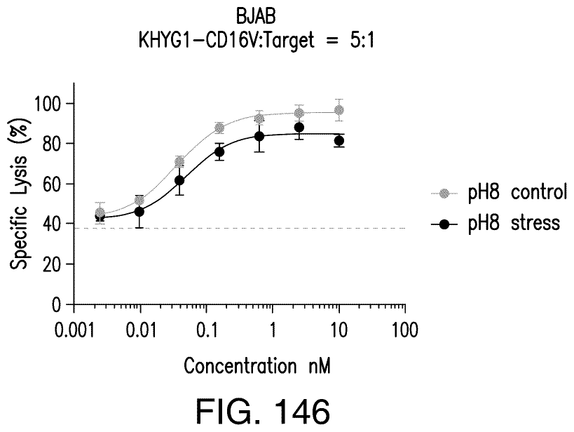

The chemical stability of AB1424/AB1612 F4 TriNKET was assessed by long term incubation at high pH (20 mM Tris, pH 8.0, 40° C., 2 weeks). A small increase in aggregation (0.2%) and minimal loss of monomer (1.3%) were observed by SEC (FIG. 141 and Table 98). A slight increase in fragmentation was detected by reduced CE-SDS (2.2%) (FIG. 142 and Table 99). After long term pH 8 stress, the charge profile monitored by cIEF demonstrated an acidic shift from 49.2% main in the control to 22.5% main in the stressed sample (FIG. 143 and Table 100). This acidic shift can be attributed to deamidation throughout the AB1424/AB1612 F4 TriNKET sequence. Deamidation is the primary chemical degradation at elevated pH. A similar acidic shift was observed for trastuzumab after the same stress conditions. Long term pH 8 stress had no significant effect on AB1424/AB1612 F4 TriNKET binding to hBAFF-R+ cells or the kinetics and affinity for hCD16aV (FIG. 144, Table 101, FIG. 145A, FIG. 145B, and Table 102). Additionally, no significant difference in potency between AB1424/AB1612 F4 TriNKET after long term pH 8 stress and control samples was observed in KHYG-1-CD16aV cytotoxicity assay (FIG. 146 and Table 103). Based on these results, it was concluded that AB1424/AB1612 F4 TRINKET is resistant to aggregation due to elevated pH stress.

TABLE 98
Summary of AB1424/AB1612 F4 TriNKET monomer, HMWS,
and LMWS after long term high pH stress.

| Test Article | Monomer (%) | HMWS (%) | LMWS (%) |
|---|---|---|---|
| AB1424/AB1612 F4 | 99.7 | 0.3 | 0.0 |
| TriNKET, pH 8 control* | | | |
| AB1424/AB1612 F4 | 98.4 | 0.5 | 1.1 |
| TriNKET, pH 8, 40° C., 2 wks | | | |

pH 8 control sample stored at −80° C. after staging, prior to analysis.

TABLE 99
Summary of AB1424/AB 1612 F4 TriNKET R CE-SDS
purity after long term high pH stress.

| Test Article | R CE-SDS purity (%) |
|---|---|
| AB1424/AB1612 F4 TriNKET, pH 8 control | 99.8 |
| AB1424/AB1612 F4 TriNKET, pH 8, 40° C., 2 | 96.6 |
| wks | |

TABLE 100
Summary of acidic, main, and basic species in AB1424/AB1612
F4 TriNKET after long term high pH stress.

| Test Article | Acidic (%) | Main (%) | Basic (%) |
|---|---|---|---|
| AB1424/AB1612 F4 | 47.8 | 49.2 | 3.0 |
| TriNKET, pH 8 control | | | |
| AB1424/AB1612 F4 | 74.5 | 22.5 | 3.0 |
| TriNKET, pH 8, 40° C., 2 | | | |
| wks | | | |

TABLE 101
Binding AB1424/AB1612 F4 TriNKET to hBAFF-R⁺
cells after long term high pH stress.

| | | Binding to |
|---|---|---|
| | | BAFF-R Expressing |
| Test Article | Target | Cells EC₅₀, nM |
| AB1424/AB1612 F4 | hBAFF-R | 0.19 |
| TriNKET, pH 8 control | | |
| AB1424/AB1612 F4 | hBAFF-R | 0.20 |
| TriNKET, pH 8, 40° C., 2 wks | | |

TABLE 102
Kinetic parameters and binding affinity of AB1424/AB1612 F4 TriNKET for
hCD16a after long term high pH stress.

| Test Article | Target | kₐ (M⁻¹s⁻¹) | k_{d} (s⁻¹) | K_{D} (nM) |
|---|---|---|---|---|
| AB1424/AB1612 F4 | hCD16aV | (1.2 ± 0.1) × 10⁵ | (1.6 ± 0.0) × 10⁻² | 137.3 ± 17.4 |
| TriNKET, pH 8 control | | | | |
| AB1424/AB1612 F4 | hCD16aV | (1.0 ± 0.0) × 10⁵ | (1.3 ± 0.0) × 10⁻² | 120.7 ± 5.0 |
| TriNKET, pH 8, 40° C., 2 | | | | |
| wks | | | | |

Results are an average of n = 3 replicates.

TABLE 103
Summary of AB1424/AB1612 F4 TriNKET EC₅₀
and maximum lysis after long term high pH exposure.

| Test article | EC₅₀ (nM) | Max killing (%) |
|---|---|---|
| AB1424/AB1612 F4 TriNKET, | 0.04 | 96 |
| pH 8 control | | |
| AB1424/AB1612 F4 TriNKET, | 0.05 | 85 |
| pH 8, 40° C., 2 wks | | |

### Manufacturability

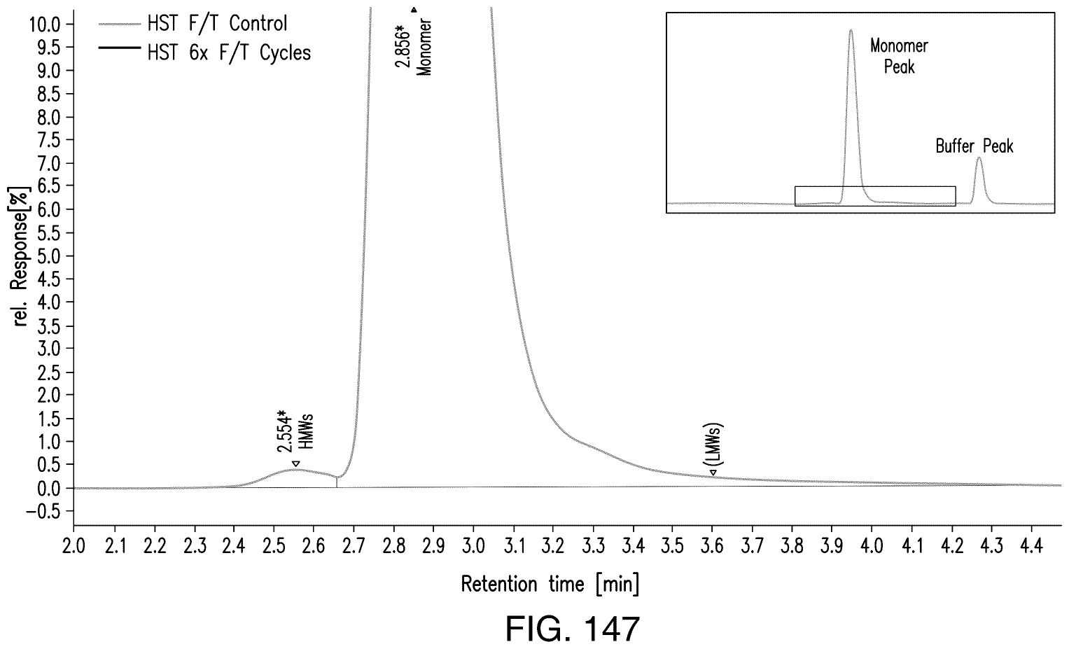

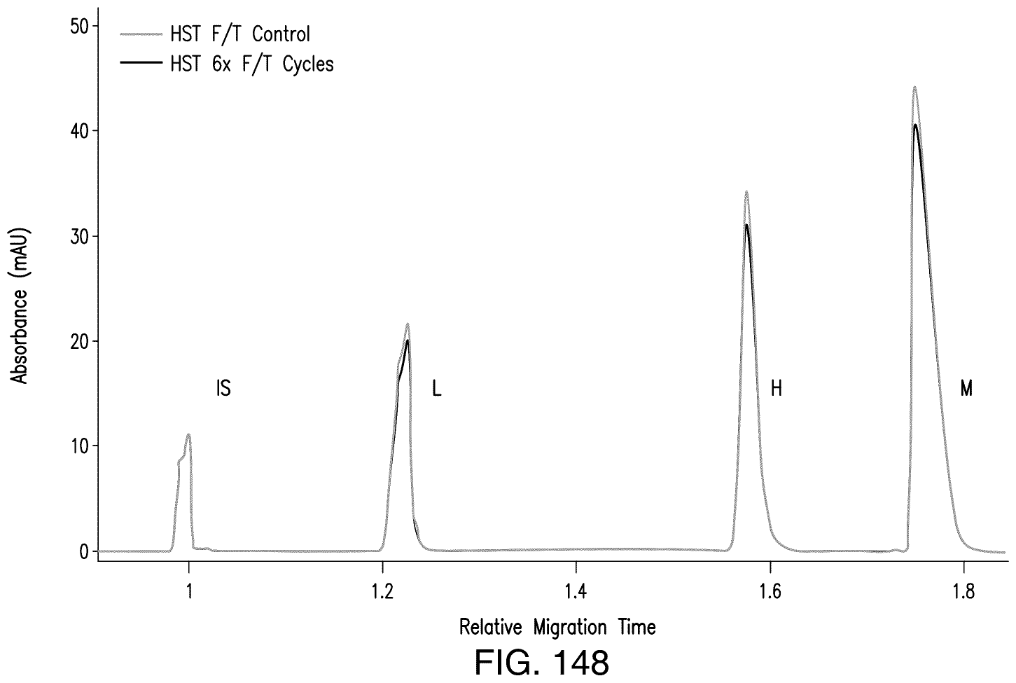

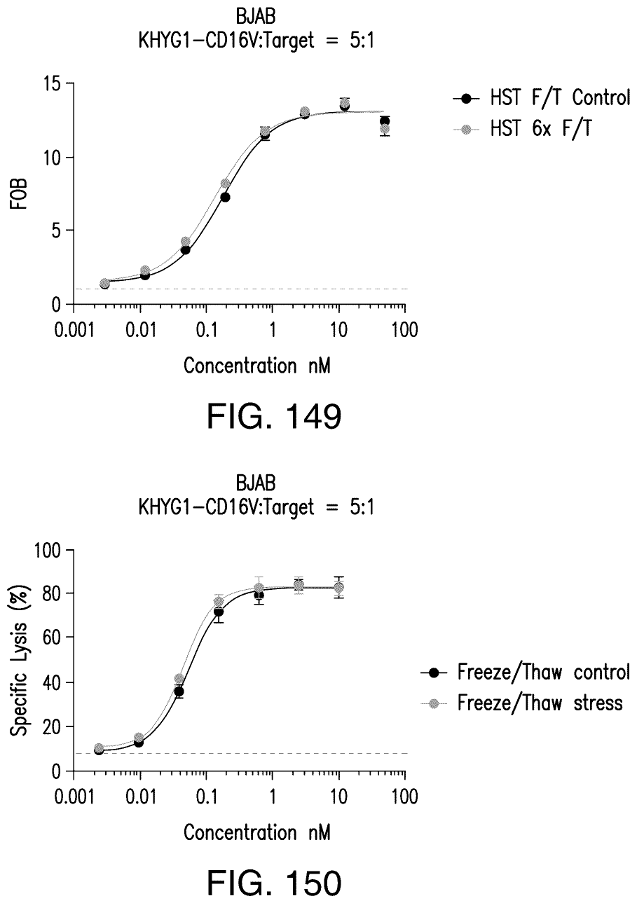

Stability during freeze/thaw (F/T) cycles is important for biotherapeutics as process intermediates and bulk drug substance may be frozen to ensure stability between process steps. The freeze/thaw stability of AB1424/AB1612 F4 TriNKET was assessed at 20 mg/ml in HST, pH 6.0. Protein concentration was assessed at the completion of the study by A280. AB1424/AB1612 F4 TriNKET concentration was 21.6 mg/ml in the control and 24.2 mg/ml after 6 freeze/thaw cycles indicating that there was no loss of protein due to the freeze/thaw stress. After six freeze/thaw cycles, the purity of AB1424/AB1612 F4 TriNKET was unchanged compared to control as assessed by SEC (FIG. 147 and Table 104) and by reduced CE-SDS (FIG. 148 and Table 105). Binding to the BAFF-R+ cell (FIG. 149 and Table 106) and potency of AB1424/AB1612 F4 TriNKET in KHYG-1-CD16aV mediated cytotoxicity assay (FIG. 150 and Table 107) remained unchanged after 6 freeze/thaw cycles compared to control sample. This indicates that AB1424/AB1612 F4 TriNKET is resistant to both aggregation and fragmentation during freeze/thaw stress.

TABLE 104
Summary of AB1424/AB1612 F4 TriNKET monomer,
HMWS, and LMWS after 6 freeze/thaw cycles.

| Test Article | Monomer (%) | HMWS (%) | LMWS (%) |
|---|---|---|---|
| AB1424/AB1612 F4 | 99.6 | 0.4 | 0.0 |
| TriNKET, F/T | | | |
| control* | | | |
| AB1424/AB1612 F4 | 99.6 | 0.4 | 0.0 |
| TriNKET, 6 F/T | | | |

*HST, pH 6.0 F/T control sample stored at −80° C. after staging, prior to analysis without F/T cycling.

TABLE 105
Summary of AB1424/AB1612 F4 TriNKET R CE-SDS
purity after freeze/thaw stress.

| Test Article | R CE-SDS purity (%) |
|---|---|
| AB1424/AB1612 F4 TriNKET, F/T control | 99.9 |
| AB1424/AB1612 F4 TriNKET, 6 F/T | 99.9 |

TABLE 106
Summary of AB1424/AB1612 F4 TriNKET binding to
BAFF-R+ cells after 6 freeze/thaw cycles.

| Test article | EC₅₀ (nM) |
|---|---|
| AB1424/AB1612 F4 TriNKET F/T control | 0.18 |
| AB1424/AB1612 F4 TriNKET F/T stress | 0.14 |

TABLE 107
Summary of AB1424/AB1612 F4 TriNKET EC₅₀ and
maximum lysis after 6 freeze/thaw cycles.

| Test article | EC₅₀ (nM) | Max killing (%) |
|---|---|---|
| AB1424/AB1612 F4 | 0.05 | 83 |
| TriNKET, F/T control | | |
| AB1424/AB1612 F4 | 0.05 | 83 |
| TriNKET, 6 F/T | | |

### Agitation

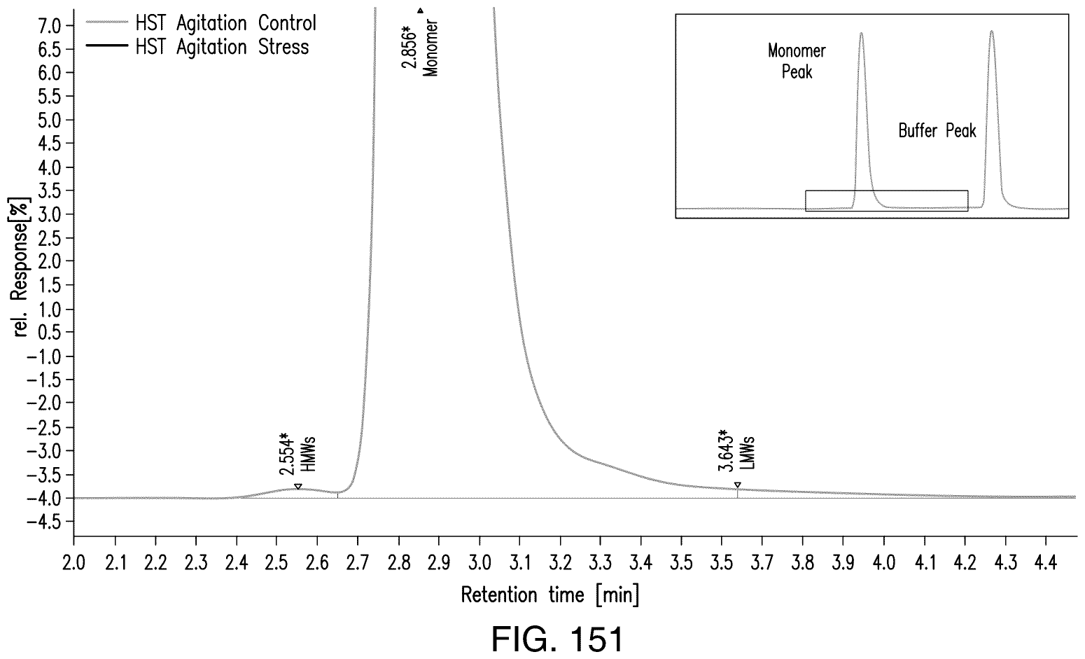

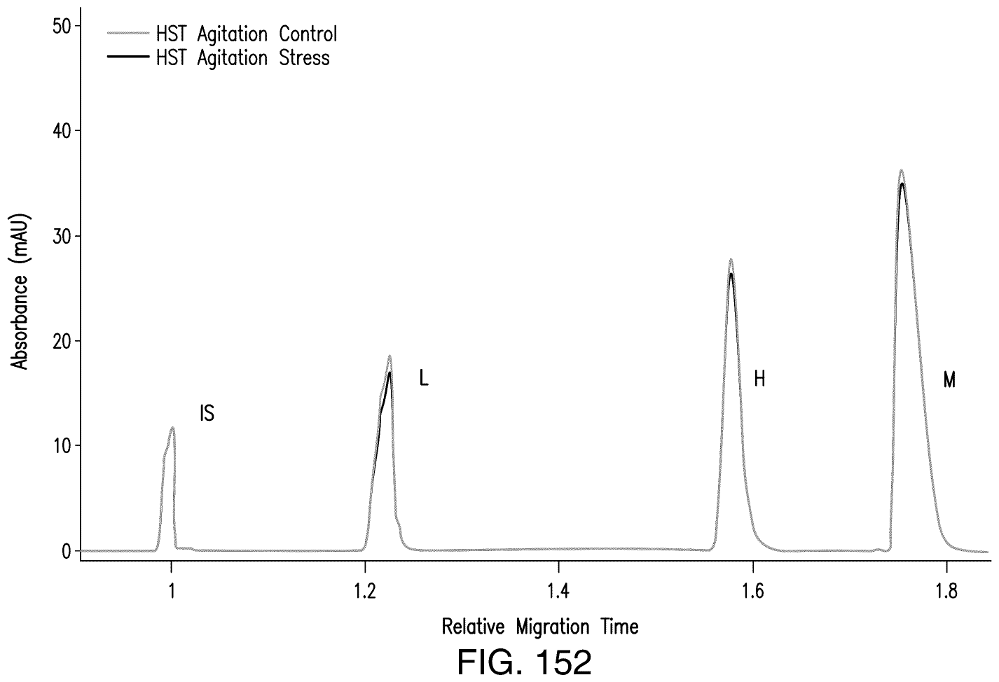

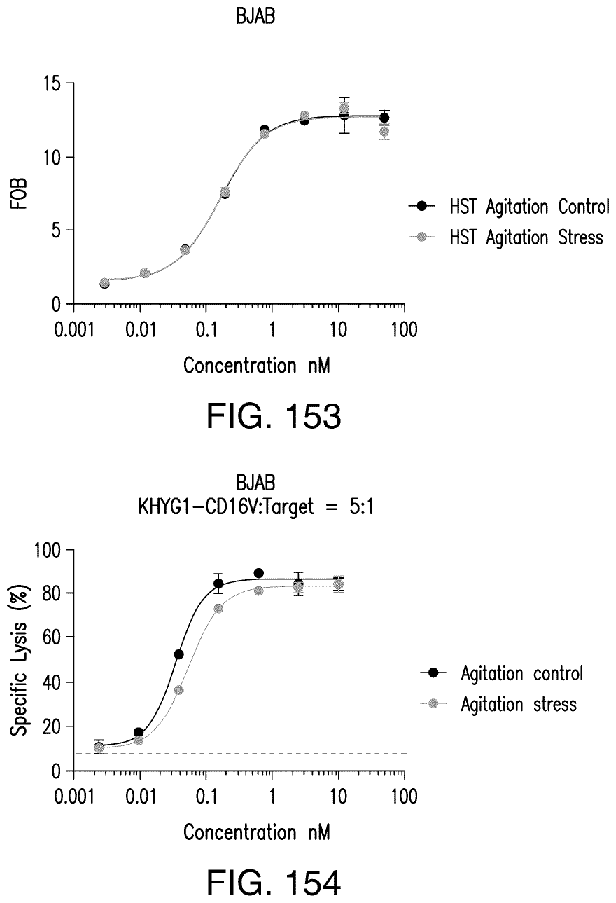

AB1424/AB1612 F4 TriNKET (at 5 mg/ml in HST, pH 6.0) was shaken at 1000 rpm at room temperature for 7 days. After agitation stress, there was no loss of monomer as detected by SEC (FIG. 151 and Table 108), no loss of purity as observed by reduced CE-SDS (FIG. 152 and Table 109) and no loss of protein concentration (Table 108). No difference between stressed and control AB1424/AB1612 F4 TriNKET samples was observed in binding to BAFF-R cells (FIG. 153 and Table 110) or in potency assessed by a KHYG-1-CD16aV mediated cytotoxicity assay. (FIG. 154 and Table 111).

TABLE 108
Summary of AB1424/AB1612 F4 TriNKET concentration
and monomer content after agitation stress.

| Test Article | Concentration (mg/mL) | Monomer (%) |
|---|---|---|
| AB1424/AB1612 F4 | 5.3 | 99.8 |
| TriNKET agitation control* | | |
| AB1424/AB1612 F4 | 5.5 | 99.8 |
| TriNKET agitation stress | | |

*HST, pH 6.0 agitation control sample was stressed at 25° C. without agitation for 1 week and stored at −80° C. after stress, prior to analysis.

TABLE 109
Summary of AB1424/AB1612 F4 TriNKET
R CE-SDS purity after agitation.

| Test Article | R CE-SDS purity (%) |
|---|---|
| AB1424/AB1612 F4 TriNKET agitation | 99.9 |
| control | |
| AB1424/AB1612 F4 TriNKET agitation | 99.9 |
| stress | |

TABLE 110
Summary of AB1424/AB1612 F4 TRINKET binding to
BAFF-R+ cells after agitation stress.

| Test article | EC₅₀ (nM) |
|---|---|
| AB1424/AB1612 F4 TRINKET agitation | 0.17 |
| control | |
| AB1424/AB1612 F4 TRINKET agitation stress | 0.16 |

TABLE 111
Potency of AB1424/AB1612 F4 TriNKET after
agitation stress compared to control.

| Test article | EC₅₀ (nM) | Max killing (%) |
|---|---|---|
| AB1424/AB1612 F4 | 0.04 | 87 |
| TriNKET agitation control | | |
| AB1424/AB1612 F4 | 0.05 | 83 |
| TriNKET agitation stress | | |

### Low pH Hold

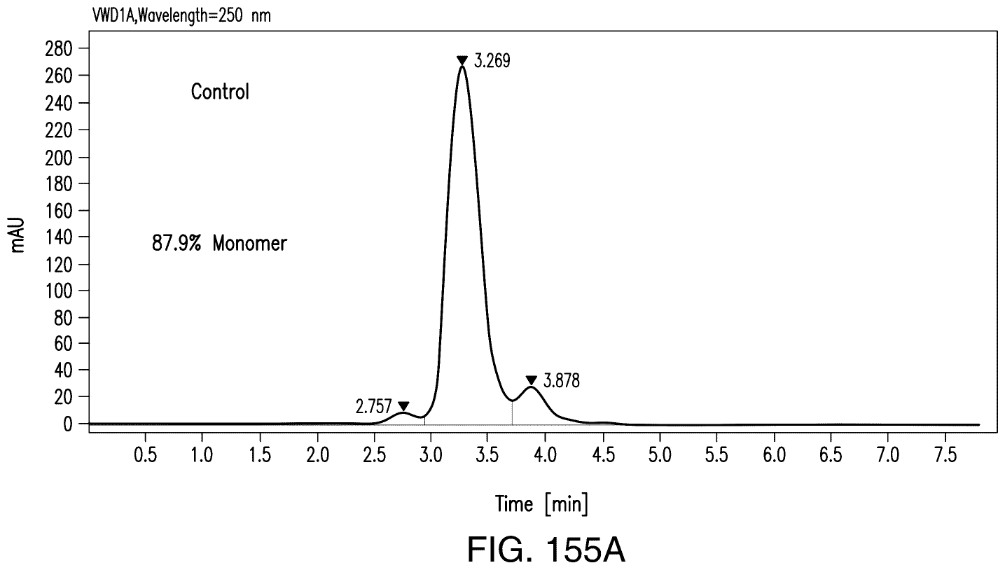

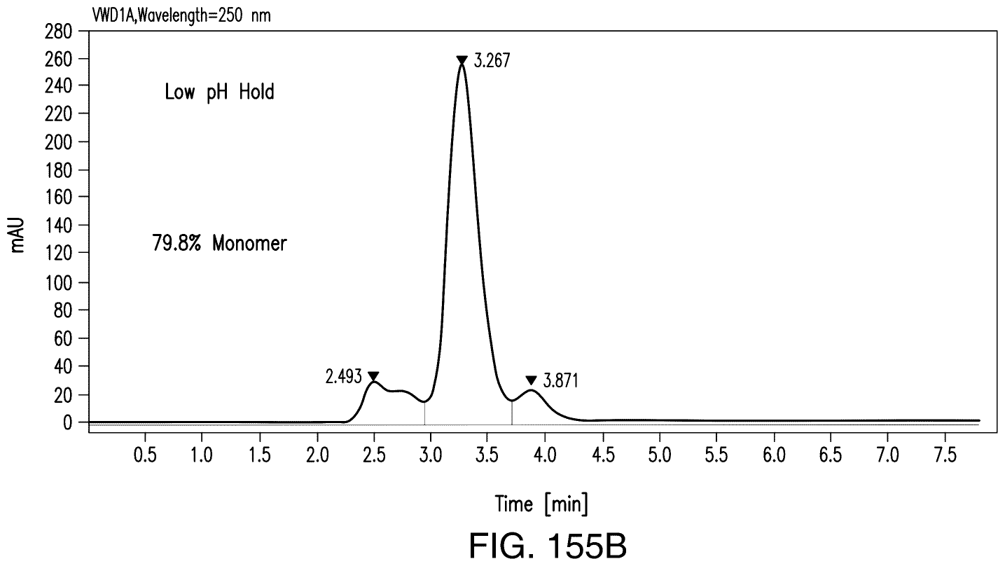

To determine if AB1424/AB1612 F4 TriNKET is amenable to a low pH hold commonly used as a viral clearance step in biologics manufacturing, AB1424/AB1612 F4 TriNKET Protein A eluate was adjusted to pH 3.51 and held for 1.5 hours at room temperature. After the hold period, the Protein A eluate was neutralized with 1.0 M Tris, pH 8.3 to achieve neutral pH. Analytical SEC was performed to determine if there were any changes in profile or aggregate content pre- and post-low pH exposure (FIG. 155A and FIG. 155B). The SEC profile of AB1424/AB1612 F4 TriNKET after a low pH hold showed an increase in HMW species and a corresponding decrease in monomer (8.1%) compared to the “no-hold” control sample, although the amount of LMW species did not change.

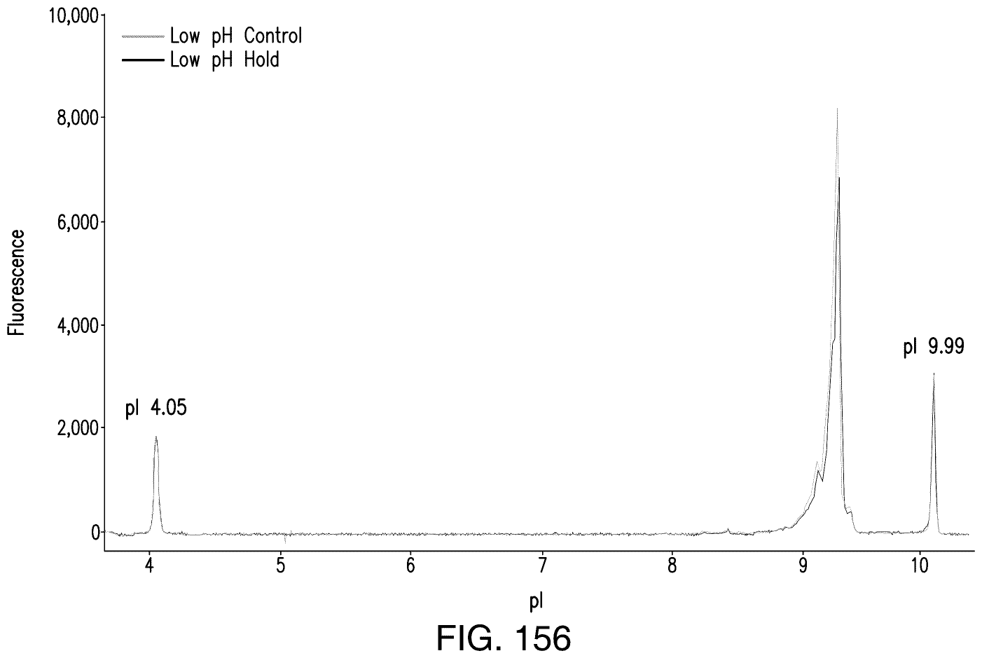

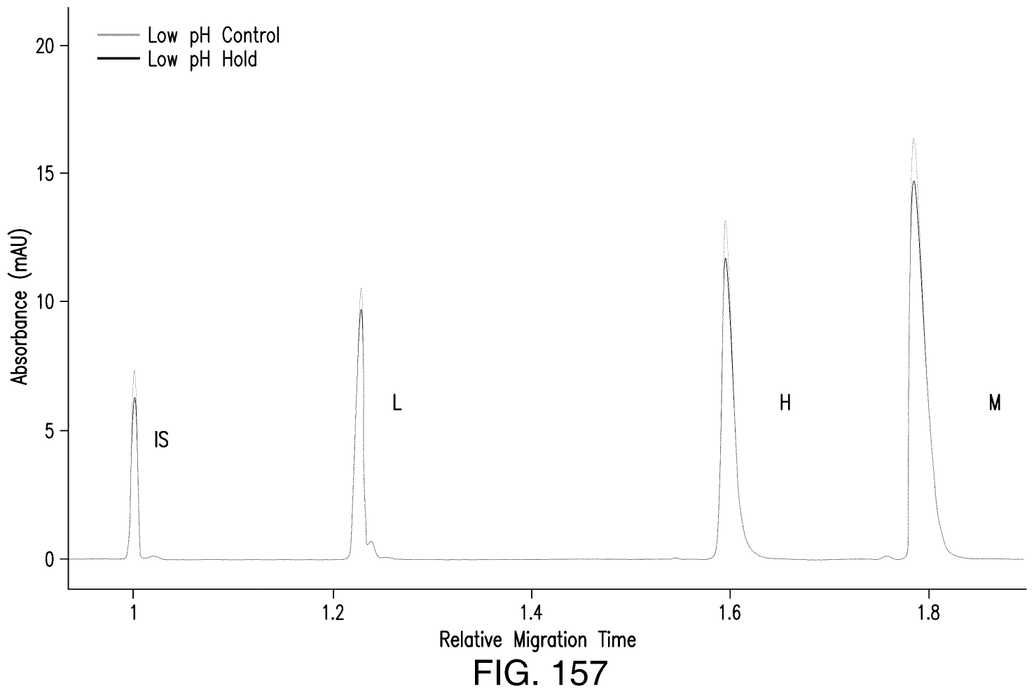

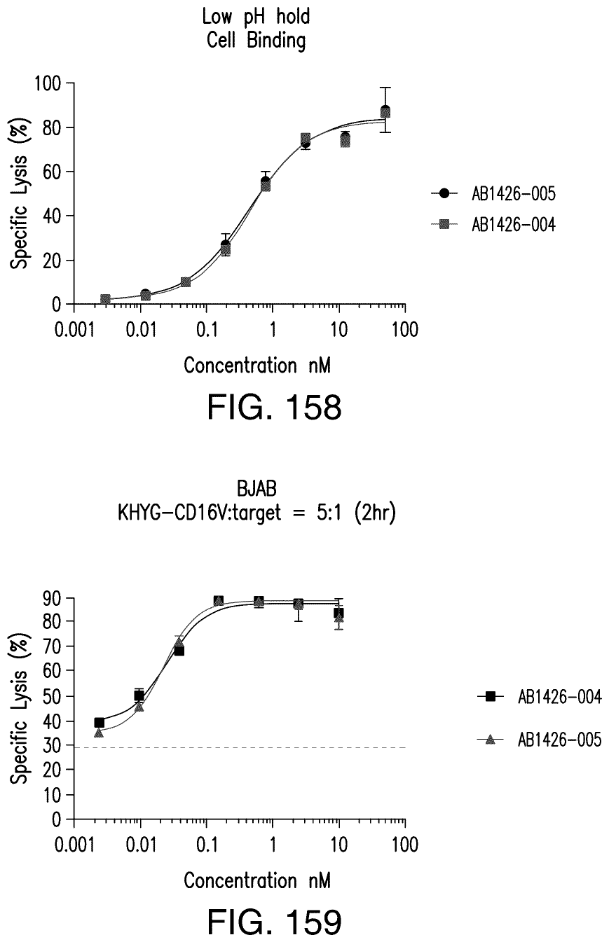

AB1424/AB1612 F4 TriNKET was further processed through ion exchange chromatography and analyzed using a panel of additional assays in comparison with purified protein that was not subjected to a low pH hold. Chemical modification of amino acid side chains can typically be observed at a global scale with cIEF. The cIEF profiles of AB1424/AB1612 F4 TriNKET control and low pH hold appear very similar and the relative quantitation of acidic, main, and basic species are all within 5% of each other (FIG. 156 and Table 112). This indicates the low pH hold did not have a measurable effect on the charge profile of AB1424/AB1612 F4 TriNKET after second step of purification. Additionally, no loss of purity was observed in fully purified AB1424/AB1612 F4 TriNKET which had undergone a low pH hold by reduced CE-SDS (FIG. 157 and Table 113). The low pH hold had no significant effect on BAFF-R+ cell binding compared to control (FIG. 158 and Table 114). Potency of the fully purified low pH hold lot of AB1424/AB1612 F4 TriNKET remained similar to the potency of the control sample as assessed by KHYG-1-CD16aV cytotoxicity assay (FIG. 159 and Table 115).

TABLE 113
Summary of fully purified AB1424/AB1612 F4 TriNKET
R CE-SDS purity after low pH hold.

| | NR CE-SDS | R CE-SDS |
|---|---|---|
| Test Article | purity (%) | purity (%) |
| AB1424/AB1612 F4 | 94.5 | 99.7 |
| TriNKET low pH Hold Control | | |
| AB1424/AB1612 F4 | 94.8 | 99.7 |
| TriNKET, low pH hold | | |

TABLE 114
BAFF-R⁺ cell binding of low pH hold lot of AB1424/AB1612
F4 TriNKET compared to control.

| | | Binding to BAFF-R |
|---|---|---|
| Test Article | Target | Expressing Cells (EC₅₀, nM) |
| AB1424/AB1612 F4 | hBAFF-R | 0.44 |
| TriNKET low pH hold | | |
| control | | |
| AB1424/AB1612 F4 | hBAFF-R | 0.46 |
| TriNKET, low pH hold | | |

Results are an average of n = 3 replicates.

TABLE 115
Potency of fully purified AB1424/AB1612 F4 TriNKET low pH hold lot
compared to control.

| Test Article | EC₅₀ (nM) | Max Killing (%) |
|---|---|---|
| AB1424/AB1612 F4 | 0.03 | 87 |
| TriNKET low pH hold control | | |
| AB1424/AB1612 F4 | 0.02 | 88 |
| TriNKET, low pH hold | | |

### Example 9—Further Analysis of AB1424/AB1612 F3′ TriNKET and AB1424/AB1612 F4 TriNKET Binding to BAFF-R

Further assessment of BAFF-R binding by AB1424/AB1612 F3′ TriNKET and AB1424/AB1612 F4 TriNKET to primary B cells and human cancer cell lines was performed. Binding experiments were performed as described in Example 4.

Antibody binding capacity of BAFF-R+ cell lines and primary B cells was performed using anti-BAFF-R mAb clone 1C11. BAFF-R expression was measured on seven human cancer cell lines as well as CHO cells engineered to express human and cynomolgus BAFF-R, results are summarized in Table 116. BAFF-R was also measured on CD19+ primary B cells in PBMC samples from three healthy donors. BAFF-R expression on primary B cells was found to be similar to expression on human cancer cell lines, as summarized in Table 117.

TABLE 116
BAFF-R quantitation on cell lines

| | Antibody binding |
|---|---|
| Cell line | capacity |
| CHO- | 239504 |
| hBAFF-R | |
| CHO- | 58922 |
| cBAFF-R | |
| BJAB | 9806 |
| Raji | 9969 |
| Ramos | 4815 |
| RL | 8007 |
| Rs4:11 | 2771 |
| Jeko1 | 10438 |
| SUDHL-6 | 14164 |

TABLE 117
BAFF-R quantitation on primary B cells

| | BAFF-R |
|---|---|
| Donor ID | ABC |
| 55212 | 12253 |
| 54136 | 4511 |
| 21189 | 9564 |

Dose-response binding of AB1424/AB1612 F3′ TriNKET, AB1424/AB1612 F4 TriNKET, their parental mAb and two isotype control TriNKETs was measured on CHO cells expressing human and cynomolgus BAFF-R. AB1424/AB1612 F3′ TriNKET had comparable, subnanomolar binding EC50s to human (0.70±0.33 nM) and cynomolgus BAFF-R (0.96±0.21 nM) expressed on CHO cells. AB1424/AB1612 F4 TriNKET and parental mAb also showed similar binding to human and cynomolgus BAFF-R, but with about 2-fold greater potency compared to AB1424/AB1612 F3′ TriNKET. AB1424/AB1612 F4 TriNKET bound human and cynomolgus BAFF-R with a potency of 0.37±0.11 nM and 0.51±0.03 nM, respectively. Parental mAb bound similarly to AB1424/AB1612 F4 TriNKET with binding potencies to human and cynomolgus BAFF-R 0.39±0.17 nM and 0.57±0.23 nM, respectively. These results demonstrate human and cynomolgus BAFF-R cross-reactive binding of AB1424/AB1612 F3′ TriNKET, AB1424/AB1612 F4 TriNKET, and their parental mAb.

Six human cancer cell lines with endogenous expression of BAFF-R were used to confirm binding observed with BAFF-R overexpressing CHO cells. Cell lines selected were of B cell origin and represent various BAFF-R+ B cell malignancies. AB1424/AB1612 F3′ TriNKET had slightly weaker potency but a high max fold over background (FOB) compared to AB1424/AB1612 F4 TriNKET and their parental mAb on 5 of the 6 cell lines tested. AB1424/AB1612 F3′ TriNKET, AB1424/AB1612 F4 TriNKET and their parental mAb bound with equivalent max FOB to Rs4;11 cells, which had the lowest BAFF-R expression. Binding EC50s and max FOB for all molecules and cell lines are summarized in Table 118.

TABLE 118
Summary of cell binding

| | AB1424/AB161 | | AB1424/AB161 | | Parental | | | | | |
|---|---|---|---|---|---|---|---|---|---|---|
| | 2 F3' TriNKET | | 2 F4 TriNKET | | mAb | | F3'-control | | F4-control | |
| | EC50 | Max | EC50 | Max | EC50 | Max | EC50 | Max | EC50 | Max |
| | nM | FOB | nM | FOB | nM | FOB | nM | FOB | nM | FOB |
| BJAB | 0.37 ± 0.16 | 16 ± 8 | 0.21 ± 0.03 | 12 ± 4 | 0.18 ± 0.04 | 12 ± 4 | ND | ND | ND | ND |
| Raji | 0.18 ± 0.01 | 10 ± 4 | 0.13 ± 0.02 | 7 ± 3 | 0.10 ± 0.01 | 0.10 ± 0.01 | ND | ND | ND | ND |
| RL | 0.27 ± 0.02 | 8 ± 2 | 0.17 ± 0.02 | 6 ± 1 | 0.14 ± 0.01 | 6 ± 1 | ND | ND | ND | ND |
| Rs4; 11 | 0.09 ± 0.02 | 4 ± 1 | 0.08 ± 0.03 | 4 ± 1 | 0.06 ± 0.02 | 3 ± 1 | ND | ND | ND | ND |
| Jeko-1 | 0.13 ± 0.03 | 7 ± 2 | 0.08 ± 0.03 | 0.08 ± 0.03 | 0.07 ± 0.02 | 5 ± 1 | ND | ND | ND | ND |
| SUDHL-6 | 0.41 ± 0.10 | 19 ± 3 | 0.18 ± 0.04 | 12 ± 3 | 0.19 ± 0.05 | 13 ± 3 | ND | ND | ND | ND |
| CHO- | 0.96 ± 0.21 | 67 ± 64 | 0.51 ± 0.03 | 56 ± 52 | 0.57 ± 0.23 | 58 ± 50 | ND | ND | ND | ND |
| cBAFF-R | | | | | | | | | | |
| CHO- | 0.70 ± 0.33 | 146 ± 78 | 0.37 ± 0.11 | 120 ± 86 | 0.39 ± 0.17 | 123 ± 85 | ND | ND | ND | ND |
| hBAFF-R | | | | | | | | | | |

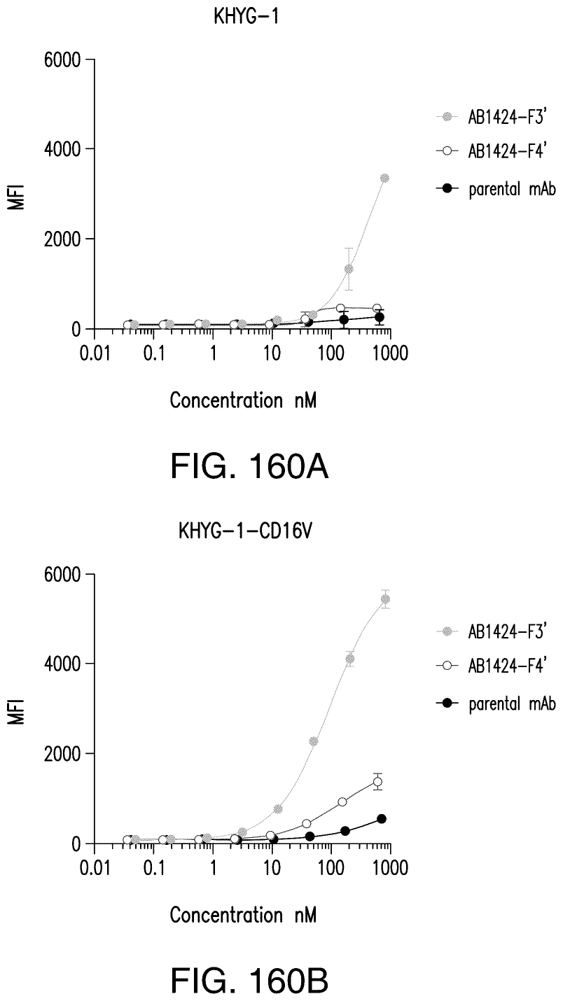

Binding of AB1424/AB1612 F3′ TriNKET and AB1424/AB1612 F4 TriNKET were compared to their parental mAb, using NK leukemia KHYG-1 cells with or without expression of the high-affinity CD16V variant. Binding patterns with both AB1424/AB1612 F3′ TriNKET and AB1424/AB1612 F4 TriNKET on KHYG-1 and KHYG-1-CD16V cell as hypothesized were observed (FIG. 160A and FIG. 160B). Weaker binding was observed on KHYG-1 parental cells lacking expression of CD16 for both AB1424/AB1612 F3′ TriNKET and AB1424/AB1612 F4 TriNKET, and no binding was observed with their parental mAb. Weaker binding of AB1424/AB1612 F4 TriNKET compared to AB1424/AB1612 F3′ TriNKET was expected and correlated with SPR affinities for these molecules binding to human NKG2D.

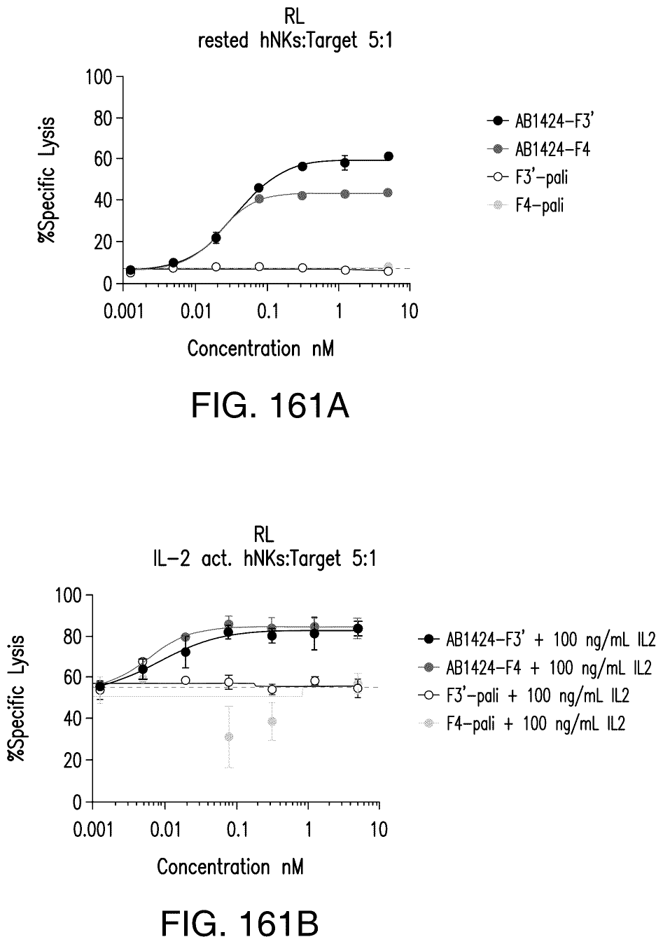

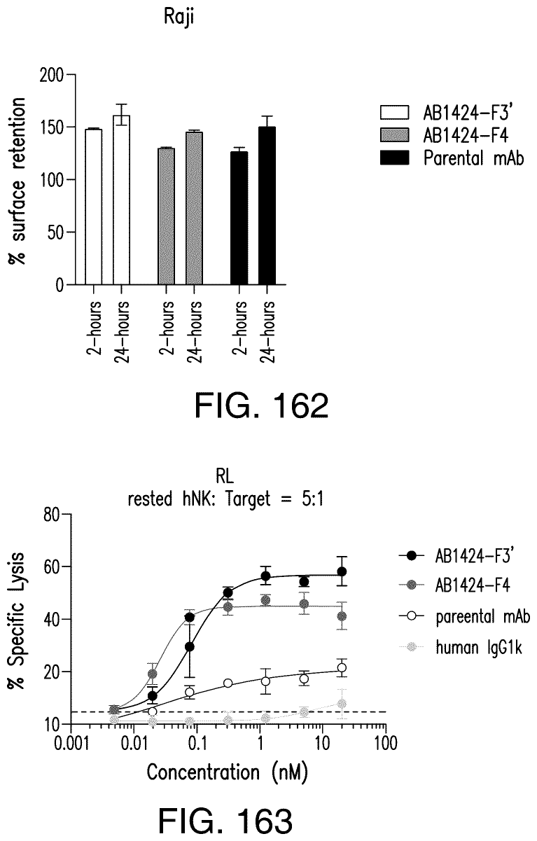

Surface retention of BAFF-R after incubation with AB1424/AB1612 F3′ TriNKET, AB1424/AB1612 F4 TriNKET or parental mAb was measured on RL and Raji cells after a 2- or 24-hour incubation. A 15-35% increase in BAFF-R surface retention after 2 hours (120%±8% and 135%±20%) was observed, which was further increased to 30-40% after a 24-hour incubation with AB1424/AB1612 F3′ TriNKET (139%±14% and 138%±33%). A similar increase at 2- and 24-hours was noted for AB1424/AB1612 F4 TriNKET and their parental mAb (FIG. 161A and FIG. 161B). Results of three independent experiments are summarized in Table 119. Similar results were observed on Raji cells (FIG. 162 and Table 120).

TABLE 119
Summary of BAFF-R cell surface retention on RL cells

| | RL 2 hr | RL24 hr |
|---|---|---|
| AB1424/AB16 | 120 ± 8 | 139 ± 14 |
| 12 F3' | | |
| TriNKET | | |
| AB1424/AB16 | 116 ± 6 | 136 ± 16 |
| 12 F4 | | |
| TriNKET | | |
| Parental mAb | 117 ± 5 | 139 ± 16 |

TABLE 120
Summary of BAFF-R cell surface retention on Raji cells

| | Raji 2 hr | Raji 24 hr |
|---|---|---|
| AB1424/AB16 | 135 ± 20 | 138 ± 33 |
| 12 F3' | | |
| TriNKET | | |
| AB1424/AB16 | 130 ± 7 | 134 ± 27 |
| 12 F4 | | |
| TriNKET | | |
| Parental mAb | 126 ± 10 | 131 ± 35 |

### Example 10—Further Analysis of AB1424/AB1612 F3′ TriNKET and AB1424/AB1612 F4 TriNKET Binding to BAFF-R

The abilities of AB1424/AB1612 F3′ TriNKET and AB1424/AB1612 F4 TriNKET to stimulate NK cell lysis of BAFF-R+ cells were tested in 2-hour short-term cytolysis assays using the Non-Hodgkin's Lymphoma (NHL) cell line, RL, as targets cells. Primary human NK cells from three healthy donors served as effector cells. AB1424/AB1612 F3′ TriNKET and AB1424/AB1612 F4 TriNKET showed superior killing potency and maximum lysis of RL target cells compared to parental mAb as seen in FIG. 163. AB1424/AB1612 F3′ TriNKET had higher maximum lysis of target cells compared to AB1424/AB1612 F4 TriNKET (44±19 and 28±15%, respectively), but with reduced killing potency (0.13±0.07 and 0.03±0.00 nM, respectively). Results from the three primary NK donors are summarized in Table 121.

TABLE 121
EC₅₀ and % Max lysis values for short-term rested NK cell lysis of RL cells

| Molecule | EC₅₀ (nM) | % Max lysis |
|---|---|---|
| AB1424/AB16 | 0.13 ± 0.07 | 44 ± 19 |
| 12 F3' | | |
| TriNKET | | |
| AB1424/AB16 | 0.03 ± 0.00 | 28 ± 15 |
| 12 F4 | | |
| TriNKET | | |
| Parental mAb | ND | ND |

ND-Not determined

AB1424/AB1612 F3′ TriNKET and AB1424/AB1612 F4 TriNKET also demonstrated strong activity in long-term 36-hour cytolysis assays using RL cells as targets cells. Primary human NK cells from three healthy donors served as effector cells. AB1424/AB1612 F3′ TriNKET and AB1424/AB1612 F4 TriNKET showed superior killing potency and maximum lysis of RL target cells compared to their parental mAb. AB1424/AB1612 F3′ TriNKET had higher maximum lysis of target cells compared to AB1424/AB1612 F4 TriNKET (44±7 and 32±13%, respectively), but with reduced killing potency (0.06±0.04 and 0.05±0.04 nM, respectively). Results from the two primary NK donors are summarized in Table 122.

TABLE 122
EC₅₀ and % Max lysis values for long-term rested NK cell lysis of RL cells

| Molecule | EC₅₀ (nM) | % Max lysis |
|---|---|---|
| AB1424/AB16 | 0.06 ± 0.04 | 44 ± 7 |
| 12 F3' | | |
| TriNKET | | |
| AB1424/AB16 | 0.04 ± 0.04 | 32 ± 13 |
| 12 F4 | | |
| TriNKET | | |
| Parental mAb | ND | ND |

ND = Not determined

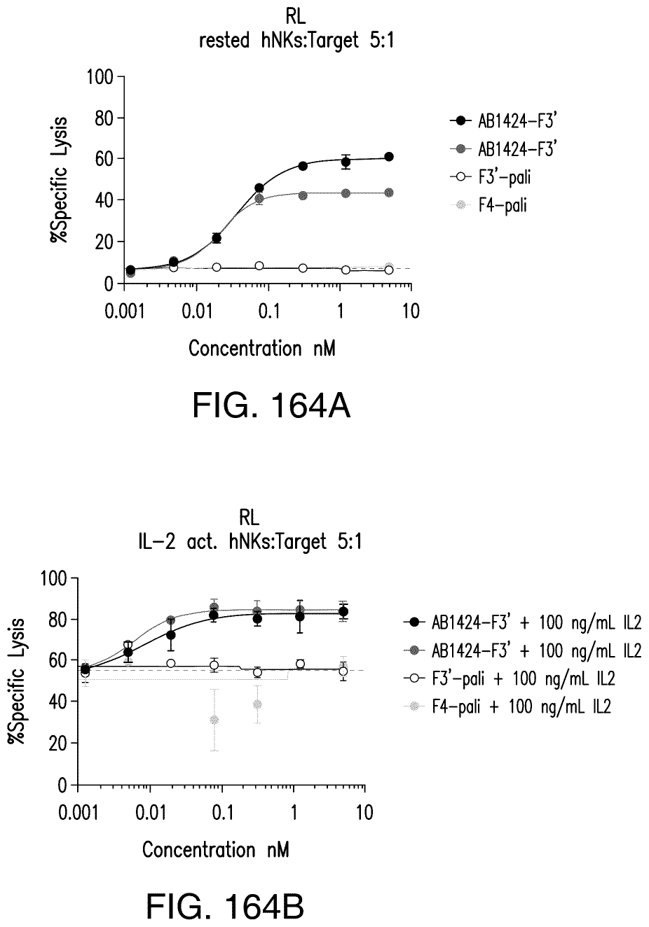

AB1424/AB1612 F3′ TriNKET and AB1424/AB1612 F4 TriNKET also enhanced cytolysis of IL-2 activated primary human NK cells against RL target cells. Human NK cells from the same donor were rested overnight or activated overnight by culture with IL-2. NK cells activated with IL-2 showed increased background killing of RL target cells in the absence of TriNKETs. AB1424/AB1612 F3′ TriNKET and AB1424/AB1612 F4 TriNKET enhanced activity of rested and IL-2-activated NK cells, but demonstrated higher maximum lysis and more potent EC₅₀ values with activated human NK cells (FIG. 164A and FIG. 164B). Data from three healthy donors are summarized in Table 123.

TABLE 123
EC50 and % Max lysis values for activated NK cells
against RL target cells

| | Rested hNK | | IL-2 act. hNK | |
|---|---|---|---|---|
| Molecule | EC50 (nM) | % Max lysis | EC50 (nM) | % Max lysis |
| AB1424/AB16 | 0.07 ± 0.03 | 45 ± 17 | 0.02 ± 0.01 | 79 ± 8 |
| 12 F3' | | | | |
| TRINKET | | | | |
| AB1424/AB16 | 0.02 ± 0.01 | 29 ± 16 | 0.01 ± 0.01 | 76 ± 13 |
| 12 F4 | | | | |
| TRINKET | | | | |

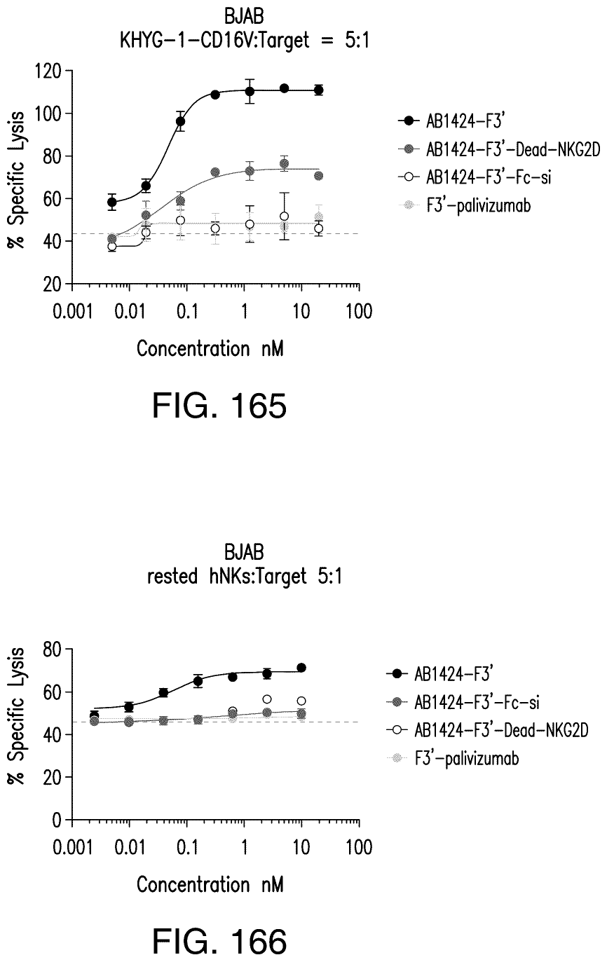

To understand the contribution of each of the TriNKET arms to the overall activity of the molecule, multiple variants of AB1424/AB1612 F3′ TriNKET were generated with mutations in the various binding arms of the molecule. In one variant, mutations were introduced into the CH2 domain of the constant region to abrogate FcγR binding; this molecule is called AB1424/AB1612 F3′ TriNKET-Fc-si. A second loss-of-function molecule was generated that abrogates NKG2D receptor binding; this molecule is called AB1424/AB1612 F3′ TriNKET-Dead-NKG2D. Lastly, a TriNKET molecule was generated that is not able to bind BAFF-R; this molecule is called F3′-palivizumab. The four molecules were first tested in target cell lysis assays using KHYG-1-CD16V effector cells. AB1424/AB1612 F3′ TriNKET was able to modulate the specific lysis of BAFF-R+ target cells in a dose-responsive manner (EC50=0.05 nM). However, no activity was observed with KHYG-1-CD16V effector cells across dose-titrations of AB1424/AB1612 F3′ TriNKET loss-of-function variants F3′-palivizumab or AB1424/AB1612 F3′ TriNKET-Fc-si. AB1424/AB1612 F3′ TriNKET-Dead-NKG2D was able to induce lysis of BJAB target cells with reduced potency and max lysis compared to AB1424/AB1612 F3′ TriNKET (EC₅₀=0.93 nM) (FIG. 165).

AB1424/AB1612 F3′ TriNKET and its loss-of-function variants were also assessed in a second assay system, where rested primary human NK cells from healthy donors were used as effector cells. Similar to the results with KHYG-1-CD16V cells, primary NK cells showed that CD16, NKG2D, and BAFF-R engagement by AB1424/AB1612 F3′ TriNKET were all required to achieve maximal NK cell responses against BJAB target cells (EC₅₀=0.06 nM) (FIG. 166).

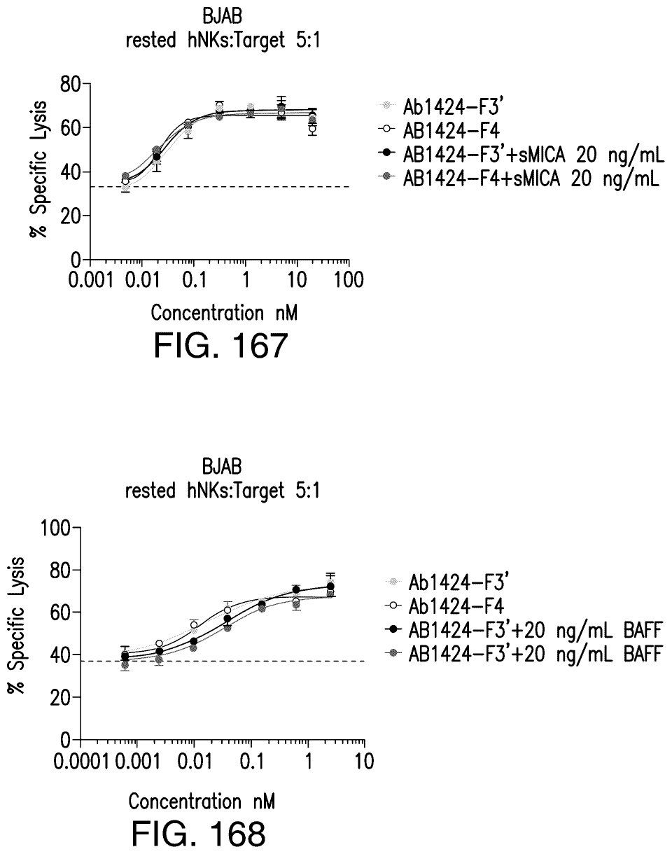

The activity of AB1424/AB1612 F3′ TriNKET was tested in the presence of a soluble NKG2D ligand. For these assays, a recombinant version of the NKG2D ligand MICA was used. MICA has broad expression across cancer indications and is known to shed from the cell surface resulting in accumulation in patient serum. Soluble MICA-Fc was added to a NK cell cytolysis assay system at 20 ng/mL, a physiologically relevant serum concentration found in cancer patients. FIG. 167 shows the dose-response curves of AB1424/AB1612 F3′ TriNKET in a primary NK cell cytolysis assay against BJAB target cells in the absence and presence of soluble MICA. The addition of MICA had no effect on the potency or maximum lysis achieved by AB1424/AB1612 F3′ TriNKET. As expected, soluble MICA also had no influence on the activity of AB1424/AB1612 F4 TriNKET. Table 124 summarizes EC₅₀ and maximum lysis values.

TABLE 124
EC₅₀ and % Max lysis values for lysis of BJAB cells
by NK cells with sMIC-A-Fc

| Molecule | EC₅₀ (nM) | % Max lysis |
|---|---|---|
| AB1424/AB16 | 0.17 ± 0.24 | 49 ± 24 |
| 12 F3' | | |
| TriNKET | | |
| AB1424/AB16 | 0.04 ± 0.02 | 44± 27 |
| 12 F4 | | |
| TriNKET | | |
| AB1424/AB16 | 0.07 ± 0.08 | 47 ± 28 |
| 12 F3' | | |
| TriNKET + | | |
| MIC-A-Fc | | |
| AB1424/AB16 | 0.05 ± 0.05 | 46 ± 27 |
| 12 F4 | | |
| TriNKET + | | |
| MIC-A-Fc | | |

AB1424/AB1612 F3′ TriNKET and AB1424/AB1612 F4 TriNKET demonstrated the ability to block BAFF binding to BAFF-R as described above. To understand the effect of soluble BAFF dimer in human NK cytolysis assays, a physiological relevant concentration of soluble BAFF, 20 ng/mL, was used. Small changes in potency were observed for AB1424/AB1612 F3′ TriNKET and AB1424/AB1612 F4 TriNKET in the presence of soluble BAFF, but the same maximal lysis was achieved (FIG. 168). Data from three donor samples are summarized in Table 125.

TABLE 125
EC₅₀ and % Max lysis values for lysis of BJAB cells by
NK cells with soluble BAFF

| Molecule | EC₅₀ (nM) | % Max lysis |
|---|---|---|
| AB1424/AB16 | 0.02 ± 0.02 | 62 ± 12 |
| 12 F3' | | |
| TriNKET | | |
| AB1424/AB16 | 0.01 ± 0.00 | 56 ± 12 |
| 12 F4 | | |
| TriNKET | | |
| AB1424/AB16 | 0.21 ± 0.15 | 64 ± 11 |
| 12 F3' | | |
| TriNKET + | | |
| MIC-A-Fcc | | |
| AB1424/AB16 | 0.04 ± 0.01 | 51 ± 15 |
| 12 F4 | | |
| TriNKET + | | |
| MIC-A-Fc | | |

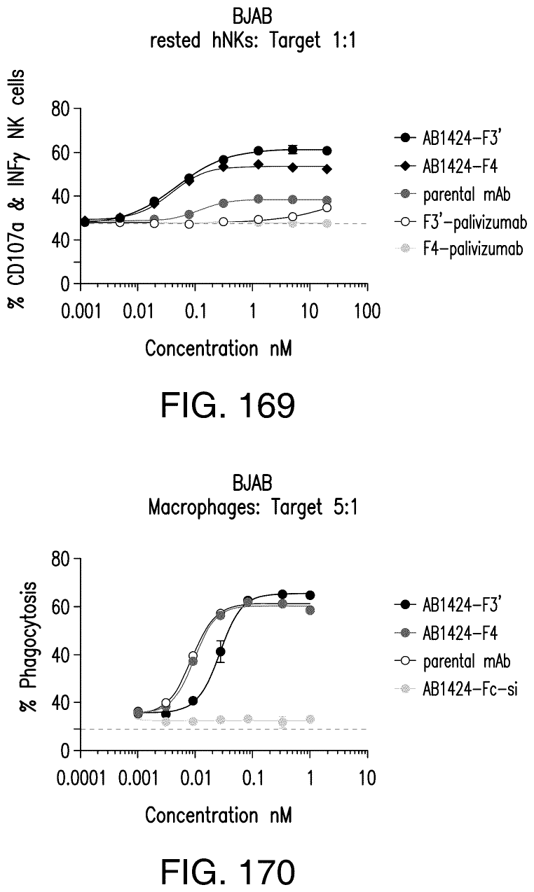

In addition to direct lysis of target cells, NK cells also produce cytokines upon activation. Thus, IFNγ production and CD107a degranulation from NK cells co-cultured with BAFF-R+ target cells in the presence of AB1424/AB1612 F3′ TriNKET, AB1424/AB1612 F4 TriNKET or their parental mAb were assessed. Rested NK cells in co-culture with BJAB target cells showed little basal induction of CD107a degranulation or intracellular IFNγ accumulation after four hours (FIG. 169). Addition of AB1424/AB1612 F3′ TriNKET to the co-cultures resulted in robust induction of degranulation and IFNγ production in a dose-responsive manner. In contrast, neither parental mAb nor non-BAFF-R-targeting TriNKET F3′-palivizumab and F4-palivizumab showed a robust increase in CD107a+IFNγ+NK cells. Assays were performed with three independent NK cell donors in co-cultures with BJAB target cells; the results are summarized in Table 126.

TABLE 126
Summary of NK cell induction of IFNγ and CD107a in
co-culture with BJAB cells

| Molecule | EC₅₀ (nM) | % Max |
|---|---|---|
| AB1424/AB16 | 0.03 ± 0.02 | 35 ± 26 |
| 12 F3' | | |
| TRINKET | | |
| AB1424/AB16 | 0.03 ± 0.01 | 32 ± 23 |
| 12 F4 | | |
| TRINKET | | |
| Parental mAb | 0.07 ± 0.05 | 23 ± 16 |

The ability of AB1424/AB1612 F3′ TriNKET to induce killing of BAFF-R+ cancer cells by cytokine-stimulated CD8+ T cells was assessed. Activated T cells showed no basal lysis of target cells, and neither the addition of AB1424/AB1612 F3′ TriNKET, AB1424/AB1612 F3′ TriNKET-Dead-NKG2D, nor F3′-palivizumab showed any triggering of CD8+ T cell activity. In contrast, a CD20-targeted tool TriNKET showed a dose-dependent induction of CD8+ T cell lysis of RL target cells, demonstrating the ability of these CD8+ cells to respond to NKG2D stimulation.

The Fc domain of a human IgG1 antibody can mediate three different types of effector functions. One type of Fc-mediated effector function is antibody-dependent cell-mediated cytotoxicity (ADCC), which is carried out by engagement of CD16 on NK cells; NK cell stimulation has been extensively characterized for AB1424/AB1612 F3′ TriNKET. A second Fc-mediated effector function is antibody-dependent cellular phagocytosis (ADCP), where macrophages attack and engulf cells coated with antibody. For AB1424/AB1612 F3′ TriNKET and AB1424/AB1612 F4 TriNKET, to assess their ability to induce ADCP of opsonized target cells, an in vitro assay system with M₀ macrophages, derived from culturing purified CD14+ monocytes with M-CSF, as effector cells, was utilized. BAFF-R+ target cells were labeled with Cell Trace CFSE dye, opsonized with test articles and co-cultured with Cell Trace Violet-labeled M₀ macrophages. Phagocytosis was analyzed by flow cytometry as Cell Trace Violet+ Cell Trace CFSE+(double-positive) events.

AB1424/AB1612 F3′ TriNKET and AB1424/AB1612 F4 TRINKET enhanced phagocytosis of BJAB target cells by M₀ macrophages. Parental mAb also showed an ability to induce M₀ macrophage phagocytosis of opsonized target cells, similar to AB1424/AB1612 F4 TriNKET (FIG. 170). AB1424/AB1612 F3′ TriNKET-Fc-si, which bears mutations in the CH2 domain to silence Fcγ-receptor binding, served as a negative control. AB1424/AB1612 F3′ TriNKET-Fc-si failed to mediate ADCP of opsonized target cells. Results using M₀ macrophages derived from three different donors are summarized in Table 127.

TABLE 127
Summary of EC₅₀ and % Max values for ADCP activity

| Molecule | EC₅₀ (nM) | % Max lysis |
|---|---|---|
| AB1424/AB16 | 0.03 ± 0.02 | 57 ± 7 |
| 12 F3' | | |
| TriNKET | | |
| AB1424/AB16 | 0.01 ± 0.00 | 53 ± 6 |
| 12 F4 | | |
| TriNKET | | |
| Parental mAb | 0.09 ± 0.00 | 54 ± 6 |

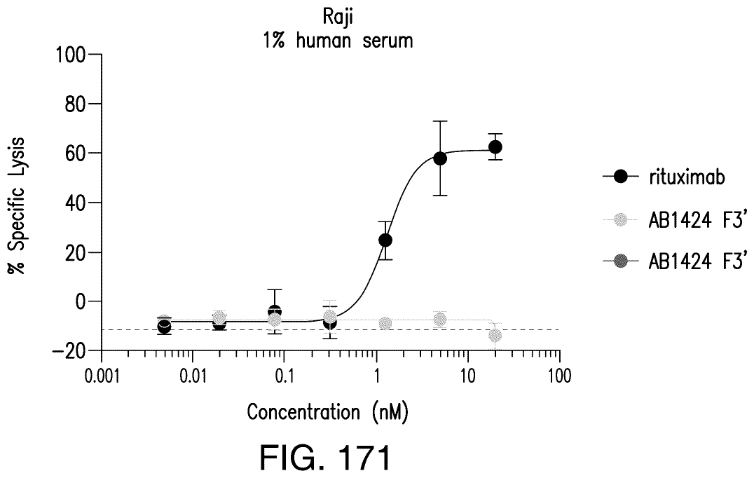

A third effector function of human IgG1 isotype antibodies is initiation of the complement cascade, leading to complement-dependent cytotoxicity (CDC). AB1424/AB1612 F3′ TriNKET and AB1424/AB1612 F4 TRINKET were constructed using a human IgG1 Fc domain; therefore, to understand the ability of AB1424/AB1612 F3′ TriNKET and AB1424/AB1612 F4 TriNKET to stimulate CDC activity, Raji cells were used in a cytotoxicity assay. Neither AB1424/AB1612 F3′ TriNKET nor AB1424/AB1612 F4 TriNKET stimulated complement-mediated killing of Raji target cells (FIG. 171). In contrast, a positive control antibody against CD20 (rituximab) showed dose-dependent lysis of Raji target cells in the presence of human serum, confirming that the serum has active complement factors.

### Example 11—Safety of AB1424/AB1612 F3′ TriNKET and AB1424/AB1612 F4 TriNKET in Human Blood

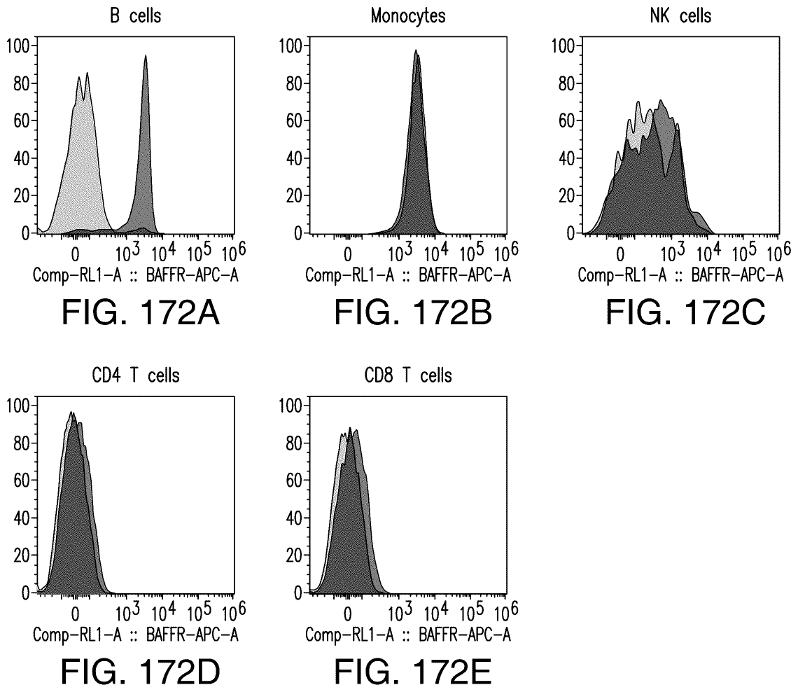

AB1424/AB1612 F3′ TriNKET binding was assessed using healthy donor peripheral blood mononuclear cells (PBMCs). Similar to results obtained with commercial antibody 11C1, AB1424/AB1612 F3′ TriNKET bound to BAFF-R+ B cells, but not to other cell subsets in PBMCs from three healthy donors (FIG. 172A-FIG. 172E).

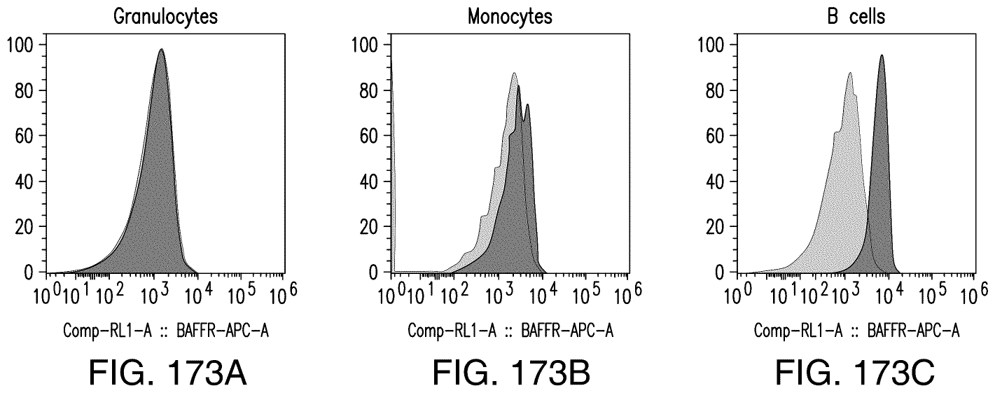

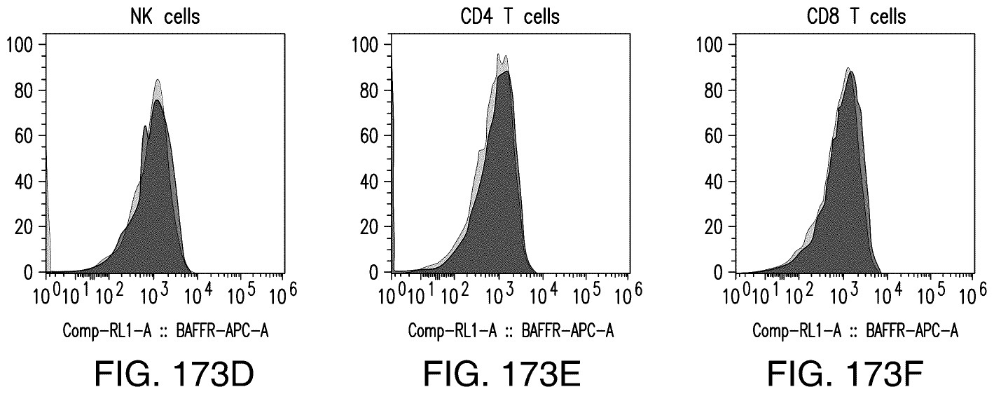

AB1424/AB1612 F3′ TriNKET binding after incubation in human whole blood samples was assessed. Immunophenotyping antibodies were used to define each of the cell populations in human blood, and AB1424/AB1612 F3′ TriNKET binding was assessed for each cell type. Consistent with the staining pattern of clone 11C1 for BAFF-R expression in human PBMC samples, staining was observed on B cells in whole blood for AB1424/AB1612 F3′ TriNKET (FIG. 173A-FIG. 173E). Appreciable binding to other cell types identified including NKG2D-positive populations such as NK cells and CD8+ T cells was not observed; the lack of appreciable binding to these subsets is consistent with AB1424/AB1612 F3′ TriNKET's low affinity design for NKG2D binding.

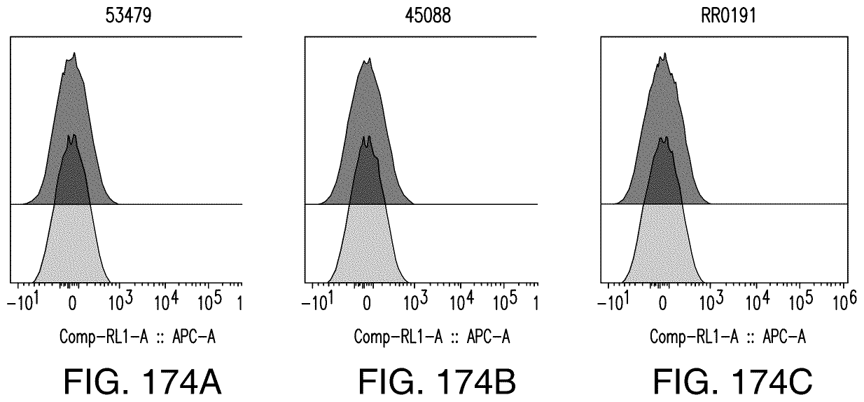

AB1424/AB1612 F3′ TriNKET binding to RBCs was analyzed. Red blood cells were identified by FACS using forward- and side-scatter plots, expression of surface CD235a and lack of CD41. No binding was observed on red blood cells for AB1424/AB1612 F3′ TriNKET (FIG. 174A-FIG. 174C) and AB1424/AB1612 F4 TriNKET. These results are consistent with lack of BAFF-R, NKG2D, and CD16 expression on RBCs.

AB1424/AB1612 F3′ TriNKET demonstrated binding in human whole blood consistent with BAFF-R expression. To further investigate the effects of AB1424/AB1612 F3′ TriNKET in whole blood samples, immune cell frequency was examined in samples treated with AB1424/AB1612 F3′ TriNKET, AB1424/AB1612 F4 TriNKET, F3′-palivizumab, F4-palivizumab or rituximab. Whole blood was exposed to 100 μg/mL of each test article and incubated for four hours before preparation of samples for FACS analysis.

Rituximab targets the cell surface antigen CD20 and has been approved for the treatment of CD20+ lymphomas. Rituximab is well characterized both in vitro and in vivo and is known to cause depletion of CD20+ cells in both human and cynomolgus monkey whole blood samples (Vugmeyster et al, 2003). Therefore, rituximab was used as a positive control in whole blood-based assays to assess cell depletion after exposure to AB1424/AB1612 F3′ TriNKET. Rituximab depleted about 50% of B cells across the three donors tested. No changes in cell frequencies were observed for other sub-populations in samples incubated with rituximab. AB1424/AB1612 F3′ TriNKET and AB1424/AB1612 F4 TriNKET caused no depletion in cell frequency as compared to control F3′-palivizumab and F4-palivizumab, respectively, in three healthy donors (FIG. 175A-FIG. 175F).

### Example 12—Analysis of AB1424/AB1612 F3′ TriNKET and AB1424/AB1612 F4 TriNKET Binding to Cynomolgus Monkey Proteins

Comparable binding affinities were observed between AB1424/AB1612 F3′ TriNKET and control hIgG1 trastuzumab against FcγRI (2.1±0.6 nM and 0.8±0.1 nM, respectively) and FcγRIII (270.8±11.0 nM and 73.7±6.8 nM, respectively) (see Table 128). There was no distinguishable difference between AB1424/AB1612 F3′ TriNKET and trastuzumab in binding affinity observed against FcRn at pH 6.0, (1.0±0.0 μM and 1.4±0.1 μM, respectively) and no detectable binding against FcRn at pH 7.4.

TABLE 128
Summary table containing affinities by SPR against
various cynomolgus FcRs

| | | AB1424/AB1612 F3' | Trastuzumab, hIgG1 |
|---|---|---|---|
| | | TriNKET | control |
| | Target | K_{D} | K_{D} |
| FcγR | FcγRI | 2.1 ± 0.6 nM | 0.8 ± 0.1 nM |
| | FcγRIII | 270.8 ± 11.0 nM | 73.7 ± 6.8 nM |
| FcRn | FcRn, pH 6.0 | 1.0 ± 0.0 μM | 1.4 ± 0.1 μM |
| | FcRn, pH 7.4 | No quantifiable binding | No quantifiable binding |

The binding of AB1424/AB1612 F3′ TriNKET to human and cynomolgus NKG2D was assessed by SPR. Two different fits were utilized to obtain the equilibrium affinity data: steady state affinity fit and kinetic fit. The kinetic constants and equilibrium affinity constants are shown in Table 129. AB1424/AB1612 F3′ TriNKET was designed to bind to cynomolgus NKG2D with low affinity with a fast rate of dissociation. The dissociation rate constant was 1.1±0.1×10⁻¹ s⁻¹ for cynomolgus NKG2D. Equilibrium affinity constants (K_{D}) obtained by kinetics fit and steady state affinity fit were very similar for cynomolgus NKG2D: 596.5±20.5 nM and 609.3±18.3 nM, respectively, which suggests high confidence in the measured parameters. Altogether, the kinetics of AB1424/AB1612 F3′ TriNKET for cynomolgus NKG2D, FcγR and BAFF-R were comparable, validating the use of cynomolgus monkey for testing AB1424/AB1612 F3′ TriNKET.

TABLE 129
Summary table containing affinities by SPR against
cynomolgus NKG2D

| | | | Kinetics Fit | Steady State |
|---|---|---|---|---|
| Test article | kₐ (M⁻¹s⁻¹) | k_{d} (s⁻¹) | K_{D} (nM) | Fit K_{D} (nM) |
| AB1424/AB1612 | 1.8 × 10⁵ | 1.1 × 10⁻¹ | 609.1 | 618.4 |
| F3' TriNKET | | | | |
| AB1424/AB1612 | 1.9 × 10⁵ | 1.2 × 10⁻¹ | 617.3 | 630.0 |
| F3' TriNKET | | | | |
| AB1424/AB1612 | 1.8 × 10⁵ | 1.1 × 10⁻¹ | 572.4 | 589.3 |
| F3' TriNKET | | | | |
| AB1424/AB1612 | 1.9 × 10⁵ | 1.1 × 10⁻¹ | 587.2 | 599.6 |
| F3' TriNKET | | | | |
| Average ± | (1.9 ± 0.1) × | (1.1 ± 0.1) × | 596.5 ± 20.5 | 609.3 ± 18.3 |
| StDev | 10⁵ | 10⁻¹ | | |

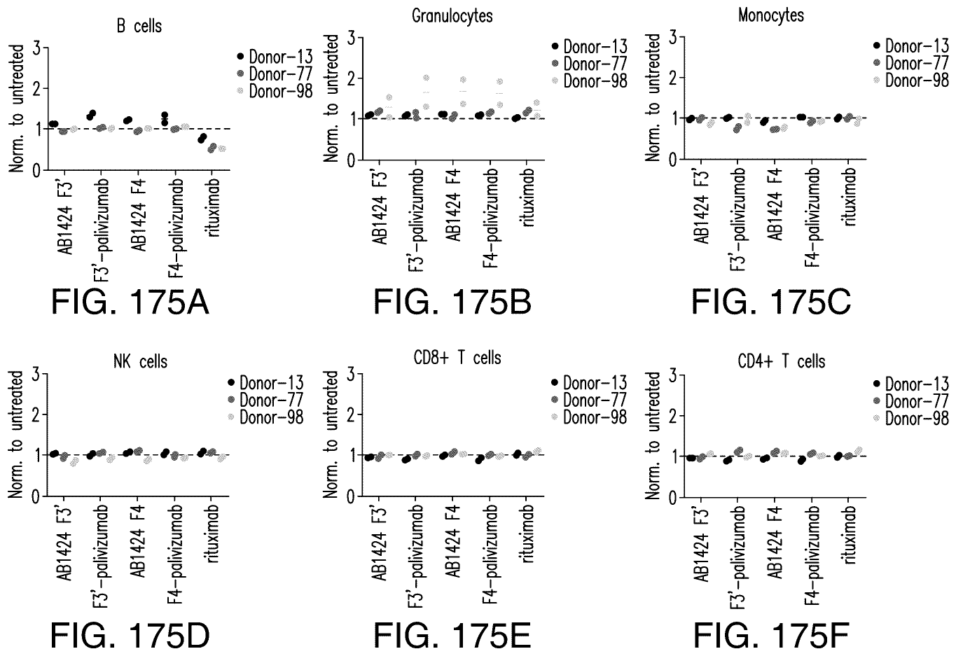

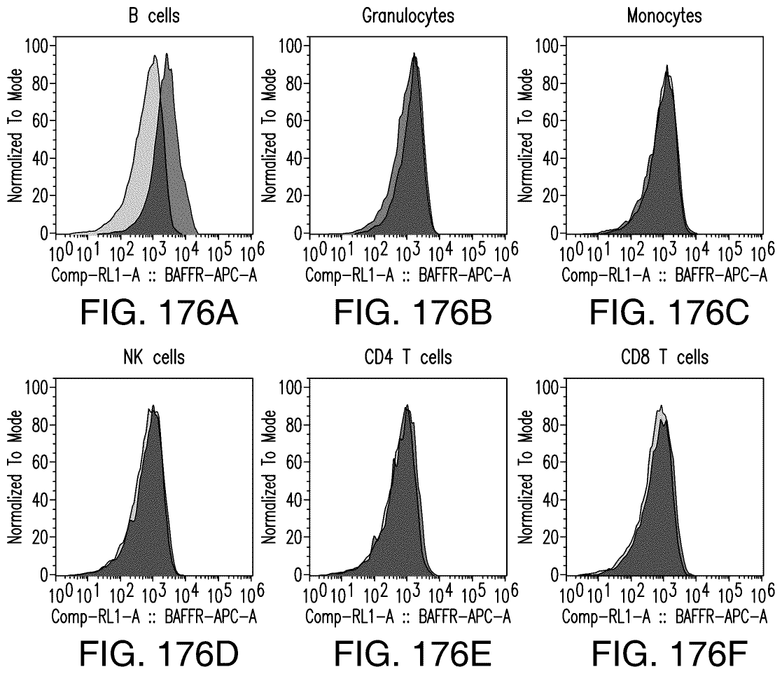

Staining in cynomolgus whole blood with AF647-conjugated AB1424/AB1612 F3′ TriNKET, AB1424/AB1612 F4 TriNKET and respective control molecules F3′-palivizumab and F4-palivizumab was measured on all immune cell subsets (representative samples shown as histograms in FIG. 176A-FIG. 176F). Non-BAFF-R targeting F3′-palivizumab and F4-palivizumab controls both had similar staining patterns as seen with AB1424/AB1612 F3′ TriNKET and AB1424/AB1612 F4 TriNKET on all non-B cell subsets. Significant and dose-dependent binding of AB1424/AB1612 F3′ TriNKET and AB1424/AB1612 F4 TriNKET was observed only on identified B cell populations.

AB1424/AB1612 F3′ TriNKET and AB1424/AB1612 F4 TriNKET demonstrated binding in cynomolgus PBMCs and whole blood to BAFF-R+ B cells. To further investigate the effects of AB1424/AB1612 F3′ TriNKET and AB1424/AB1612 F4 TriNKET in whole blood samples, immune cell frequencies were examined in samples treated with AB1424/AB1612 F3′ TriNKET and AB1424/AB1612 F4 TriNKET. Non-BAFF-R targeting control TriNKETs, F3′-palivizumab and F4-palivizumab were used as negative controls. Whole blood was exposed to 100 μg/mL of each test article and incubated for four hours before preparation of samples for FACS analysis.

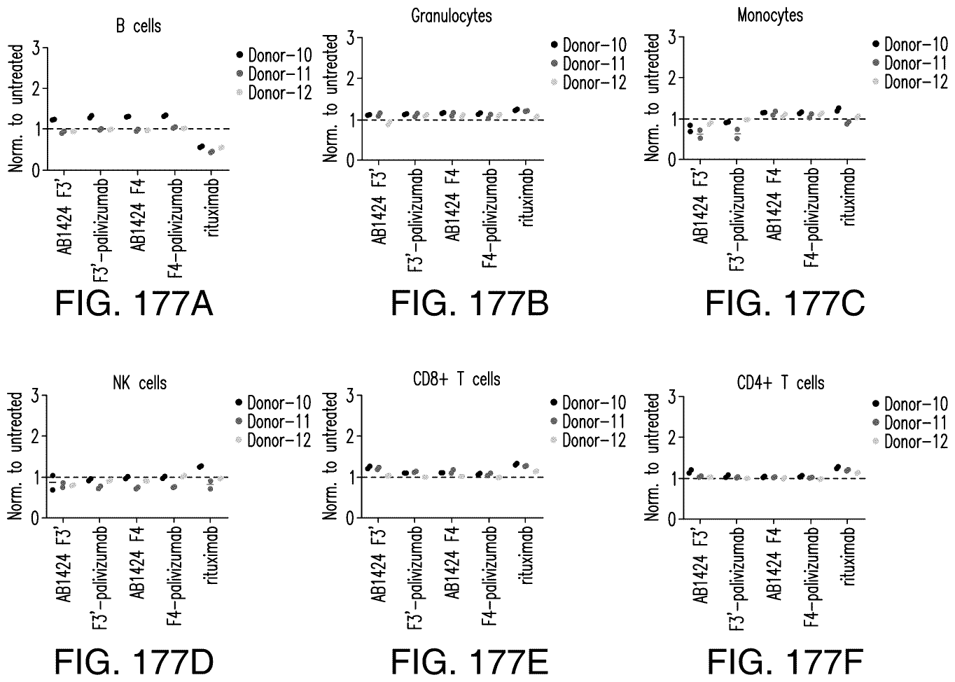

Rituximab targets the cell surface antigen CD20 and has been approved for the treatment of CD20+ lymphomas. Rituximab is well characterized both in vitro and in vivo and is known to cause depletion of CD20+ cells in both human and cynomolgus monkey whole blood samples (Vugmeyster et al, 2003). Therefore, rituximab was used as a positive control in whole blood-based assays to assess B cell depletion after exposure to AB1424/AB1612 F3′ TriNKET and AB1424/AB1612 F4 TriNKET. Rituximab demonstrated about 50% depletion of B cells across the three donors tested. No changes in cell frequencies were observed for other subpopulations in samples incubated with rituximab, suggesting target specificity for depletion. AB1424/AB1612 F3′ TriNKET and AB1424/AB1612 F4 TriNKET did not cause changes in cell frequencies compared to F3′-palivizumab and F4-palivizumab controls in any of the samples from three healthy animals (FIG. 177A-FIG. 177F).

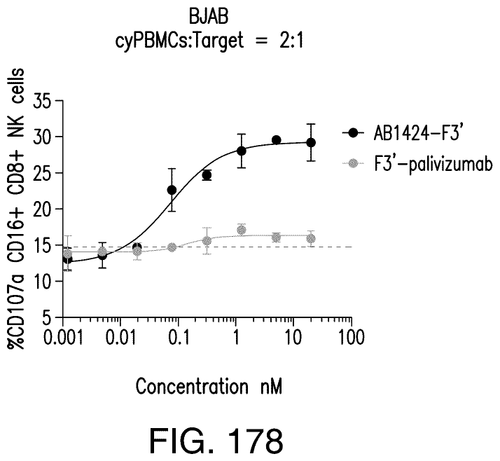

The ability of AB1424/AB1612 F3′ TriNKET to enhance activation of cynomolgus monkey NK cells was assessed in a co-culture assay with human lymphoma cell line, BJAB, that endogenously expresses BAFF-R. NKG2D expression was consistently found on CD8+NK cells but not on CD8− NK cells. A staining and gating strategy was used with CD45+CD14−CD20−CD3−CD8+CD16+ to define CD8+NK cells in which responses to BAFF-R targeting TriNKETs were predicted. AB1424/AB1612 F3′ TriNKET showed superior activity in enhancing degranulation of CD8+NK cells from two cynomolgus PBMC samples tested when compared to F3′-palivizumab (representative plot shown in FIG. 178; summary in Table 130).

Overall, the potency of AB1424/AB1612 F3′ TriNKET in stimulating NK cell degranulation (CD107a+) was comparable between cynomolgus and human activation assays (cynomolgus NK cells EC₅₀=0.19±0.16 nM and human NK cells EC₅₀=0.03±0.02 nM).

TABLE 130
EC50 values for degranulation of cynomolgus NK
cells in co-cultures with BJAB target cells

| Molecule | EC₅₀ (nM) | Max % CD107a |
|---|---|---|
| F3\grave{ }-palivizumab | N/D | N/D |
| AB1424/AB1612 | 0.19 ± 0.16 | 23 ± 9 |
| F3' TriNKET | | |

N/D = Not determined

### INCORPORATION BY REFERENCE

Unless stated to the contrary, the entire disclosure of each of the patent documents and scientific articles referred to herein is incorporated by reference for all purposes.

### EQUIVALENTS

The application may be embodied in other specific forms without departing from the spirit or essential characteristics thereof. The foregoing embodiments are therefore to be considered in all respects illustrative rather than limiting the application described herein. Scope of the application is thus indicated by the appended claims rather than by the foregoing description, and all changes that come within the meaning and range of equivalency of the claims are intended to be embraced therein.

What is claimed is:

1. A protein comprising:
(a) a first antigen-binding site that binds NKG2D;
(b) a second antigen-binding site that binds B cell-activating factor receptor (BAFF-R); and
(c) an antibody Fc domain or a portion thereof sufficient to bind CD16, or a third antigen-binding site that binds CD16.

2. The protein of claim 1, wherein the first antigen-binding site that binds NKG2D is a Fab fragment, and the second antigen-binding site that binds BAFF-R is an scFv.

3. The protein of claim 1, wherein the first antigen-binding site that binds NKG2D is an scFv, and the second antigen-binding site that binds BAFF-R is a Fab fragment.

4. The protein of claim 1, further comprising an additional antigen-binding site that binds BAFF-R.

5. The protein of claim 4, wherein the first antigen-binding site that binds NKG2D is an scFv, and the second and the additional antigen-binding sites that bind BAFF-R are each a Fab fragment.

6. The protein of claim 4, wherein the first antigen-binding site that binds NKG2D is an scFv, and the second and the additional antigen-binding sites that bind BAFF-R are each an scFv.

7. The protein of any one of claims 4-6, wherein the amino acid sequences of the second and the additional antigen-binding sites are identical.

8. The protein of any one of claims 3 and 6-7, wherein the scFv that binds NKG2D is linked to an antibody constant domain or a portion thereof sufficient to bind CD16 via a hinge comprising Ala-Ser or Gly-Ser, and wherein the scFv comprises a heavy chain variable domain and a light chain variable domain.

9. The protein of any one of claims 2 and 6-8, wherein each scFv that binds BAFF-R is linked to an antibody constant domain or a portion thereof sufficient to bind CD16 via a hinge comprising Ala-Ser or Gly-Ser, and wherein the scFv comprises a heavy chain variable domain and a light chain variable domain.

10. The protein of claim 8 or 9, wherein the hinge further comprises an amino acid sequence Thr-Lys-Gly.

11. The protein of any one of claims 3 and 5-10, wherein within the scFv that binds NKG2D, the heavy chain variable domain of the scFv forms a disulfide bridge with the light chain variable domain of the scFv.

12. The protein of any one of claims 2 and 6-11, wherein within each scFv that binds BAFF-R, the heavy chain variable domain of the scFv forms a disulfide bridge with the light chain variable domain of the scFv.

13. The protein of claim 11 or 12, wherein the disulfide bridge is formed between C44 of the heavy chain variable domain and C100 of the light chain variable domain, numbered under the Kabat numbering scheme.

14. The protein of any one of claims 3 and 5-13, wherein within the scFv that binds NKG2D, the heavy chain variable domain is linked to the light chain variable domain via a flexible linker.

15. The protein of any one of claims 2 and 6-14, wherein within each scFv that binds BAFF-R, the heavy chain variable domain is linked to the light chain variable domain via a flexible linker.

16. The protein of claim 14 or 15, wherein the flexible linker comprises (G₄S)₄ (SEQ ID NO:119).

17. The protein of any one of claims 3 and 5-16, wherein within the scFv that binds NKG2D, the heavy chain variable domain is positioned at the C-terminus of the light chain variable domain.

18. The protein of any one of claims 2 and 6-17, wherein within each scFv that binds BAFF-R, the heavy chain variable domain is positioned at the C-terminus of the light chain variable domain.

19. The protein of any one of claims 3 and 5-16, wherein within the scFv that binds NKG2D, the heavy chain variable domain is positioned at the N-terminus of the light chain variable domain.

20. The protein of any one of claims 2, 6-17, and 19, wherein within each scFv that binds BAFF-R, the heavy chain variable domain is positioned at the N-terminus of the light chain variable domain.

21. The protein of any one of claims 2, 9-10, 12-13, 15-16, 18 and 20, wherein the Fab fragment that binds NKG2D is not positioned between an antigen-binding site and the Fc or the portion thereof.

22. The protein of any one of claims 3, 5, 7-8, 10-11, 13-14, 16-17, and 19, wherein no Fab fragment that binds BAFF-R is positioned between an antigen-binding site and the Fc or the portion thereof.

23. A protein comprising:
(a) a first antigen-binding site comprising a Fab fragment that binds NKG2D;
(b) a second antigen-binding site comprising a single-chain variable fragment (scFv) that binds B cell-activating factor receptor (BAFF-R); and
(c) an Fc domain comprising a first antibody constant domain and a second antibody constant domain that form a heterodimer that binds CD16,
wherein the scFv is linked to the N-terminus of the first antibody constant domain via a hinge, and the Fab is linked to the N-terminus of the second antibody constant domain.

24. The protein of claim 23, wherein the hinge comprises Gly-Ser.

25. The protein of any one of claims 1-24, wherein the first antigen-binding site binds human NKG2D.

26. The protein of any one of claims 1-25, wherein the first antigen-binding site that binds NKG2D comprises a VH comprising complementarity-determining region 1 (CDR1), complementarity-determining region 2 (CDR2), and complementarity-determining region 3 (CDR3) comprising the amino acid sequences of SEQ ID NOs: 81, 82, and 112, respectively; and a VL comprising CDR1, CDR2, and CDR3 comprising the amino acid sequences of SEQ ID NOs: 86, 77, and 87, respectively.

27. The protein of any one of claims 1-26, wherein the first antigen-binding site that binds NKG2D comprises a VH comprising CDR1, CDR2, and CDR3 sequences represented by the amino acid sequences of SEQ ID NOs: 81, 82, and 97, respectively; and a VL comprising CDR1, CDR2, and CDR3 sequences represented by the amino acid sequences of SEQ ID NOs: 86, 77, and 87, respectively.

28. The protein of any one of claims 1-27, wherein the first antigen-binding site that binds NKG2D comprises a VH comprising an amino acid sequence at least 90% identical to SEQ ID NO:95 and a VL comprising an amino acid sequence at least 90% identical to SEQ ID NO:85.

29. The protein of any one of claims 1-28, wherein the first antigen-binding site that binds NKG2D comprises a VH comprising an amino acid sequence of SEQ ID NO:95 and a VL comprising an amino acid sequence of SEQ ID NO:85.

30. The protein of any one of claims 1-29, wherein the second antigen-binding site comprises a heavy chain variable domain comprising CDR1, CDR2, and CDR3 sequences of SEQ ID NOs: 260, 249, and 261, respectively; and a light chain variable domain comprising CDR1, CDR2, and CDR3 sequences of SEQ ID NOs: 217, 77, and 259, respectively.

31. The protein of any one of claims 1-30, wherein the second antigen-binding site comprises a heavy chain variable domain comprising CDR1, CDR2, and CDR3 sequences of SEQ ID NOs: 214, 233, and 248, respectively; and a light chain variable domain comprising CDR1, CDR2, and CDR3 sequences of SEQ ID NOs: 217, 77, and 249, respectively.

32. The protein of claim 31, wherein the second antigen-binding site comprises a heavy chain variable domain at least 90% identical to SEQ ID NO:250 and a light chain variable domain at least 90% identical to SEQ ID NO:251.

33. The protein of claim 32, wherein the second antigen-binding site comprises a VH with a G44C substitution relative to SEQ ID NO:250, and a VL with a G100C substitution relative to SEQ ID NO:251.

34. The protein of any one of claims 1-33, wherein the second antigen-binding site comprises a VH comprising the amino acid sequence of SEQ ID NO:252 and a VL comprising the amino acid sequence of SEQ ID NO:253, or a VH comprising the amino acid sequence of SEQ ID NO:250 and a VL comprising the amino acid sequence of SEQ ID NO:251.

35. The protein of any one of claims 1-34, wherein the second antigen-binding site comprises a VH comprising the amino acid sequence of SEQ ID NO:252 and a VL comprising the amino acid sequence of SEQ ID NO:253.

36. The protein of any one of claims 1-32 or 34, wherein the second antigen-binding site comprises a VH comprising the amino acid sequence of SEQ ID NO:250 and a VL comprising the amino acid sequence of SEQ ID NO:251.

37. The protein of any one of claims 1-35, wherein the second antigen-binding site comprises a single-chain fragment variable (scFv), and wherein the scFv comprises a VH comprising the amino acid sequence of SEQ ID NO:252 and a VL comprising the amino acid sequence of SEQ ID NO:253.

38. The protein of any one of claims 1, 2, 9-10, 12-13, 15-16, 18, 20-21, 23-35 and 37, wherein the second antigen-binding site comprises a single-chain fragment variable (scFv), and wherein the scFv comprises an amino acid sequence at least 90% identical to a sequence selected from the group consisting of SEQ ID NOs: 254 and 255.

39. The protein of any one of claims 1, 2, 9-10, 12-13, 15-16, 18, 20-21, 23-35 and 37-38, wherein the second antigen-binding site comprises an scFv and the scFv comprises an amino acid sequence at least 90% identical to SEQ ID NO:254.

40. The protein of any one of claims 1, 2, 9-10, 12-13, 15-16, 18, 20-21, 23-35 and 37-39, wherein the second antigen-binding site comprises an scFv and the scFv comprises an amino acid sequence of SEQ ID NO:254.

41. The protein of any one of claims 1, 2, 9-10, 12-13, 15-16, 18, 20-21, 23-35 and 37-40, wherein the protein comprises an amino acid sequence at least 90% identical to SEQ ID NO:270.

42. The protein of any one of claims 1, 2, 9-10, 12-13, 15-16, 18, 20-21, 23-35 and 37-41, wherein the protein comprises an amino acid sequence of SEQ ID NO:270.

43. The protein of any one of claims 1-3, 5, 7-8, 10-11, 13-14, 16-17, 19, 24-34 and 36, wherein the protein comprises an amino acid sequence at least 90% identical to SEQ ID NO:271.

44. The protein of any one of claims 1-3, 5, 7-8, 10-11, 13-14, 16-17, 19, 24-34, 36, and 43, wherein the protein comprises the amino acid sequence of SEQ ID NO:271.

45. The protein of any one of claims 1-44, wherein the second antigen-binding site binds human BAFF-R with a dissociation constant (K_{D}) smaller than or equal to 5 nM, as measured by surface plasmon resonance (SPR).

46. The protein of any one of claims 1-45, wherein the second antigen-binding site inhibits binding of BAFF-R to BAFF.

47. A protein comprising:
(a) a first antigen-binding site comprising a VH and a VL of an anti-NKG2D antibody, wherein the VH comprises the amino acid sequence of SEQ ID NO:95 and the VL comprises the amino acid sequence of SEQ ID NO:85;
(b) a second antigen-binding site comprising a VH and a VL of an anti-BAFF-R antibody, wherein the VH comprises the amino acid sequence of SEQ ID NO:252 and the VL comprises the amino acid sequence of SEQ ID NO:253; and
(c) an antibody Fc domain or a portion thereof sufficient to bind CD16, or a third antigen-binding site that binds CD16.

48. A protein comprising:
(a) a first antigen-binding site comprising a VH and a VL of an anti-NKG2D antibody, wherein the VH comprises the amino acid sequence of SEQ ID NO:95 and the VL comprises the amino acid sequence of SEQ ID NO:85;
(b) a second antigen-binding site comprising the amino acid sequence of SEQ ID NO:254; and
(c) an antibody Fc domain or a portion thereof sufficient to bind CD16, or a third antigen-binding site that binds CD16.

49. The protein of any one of claims 1-48, wherein the antibody Fc domain is a human IgG1 antibody Fc domain.

50. The protein of claim 49, wherein the antibody Fc domain or a portion thereof comprises an amino acid sequence at least 90% identical to SEQ ID NO:118.

51. The protein of claim 49 or 50, wherein at least one polypeptide chain of the antibody Fc domain comprises one or more mutations, relative to SEQ ID NO:118, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system.

52. The protein of any one of claims 49-51, wherein at least one polypeptide chain of the antibody Fc domain comprises one or more mutations, relative to SEQ ID NO:118, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, F405L, Y407A, Y407I, Y407V, K409F, K409W, K409D, K409R, T411D, T411E, K439D, and K439E, numbered according to the EU numbering system.

53. The protein of any one of claims 49-52, wherein one polypeptide chain of the antibody heavy chain constant region comprises one or more mutations, relative to SEQ ID NO:118, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:118, at one or more positions selected from Q347, Y349, L351, S354, E356, E357, S364, T366, L368, K370, N390, K392, T394, D399, D401, F405, Y407, K409, T411, and K439, numbered according to the EU numbering system.

54. The protein of claim 53, wherein one polypeptide chain of the antibody heavy chain constant region comprises K360E and K409W substitutions relative to SEQ ID NO:118; and the other polypeptide chain of the antibody heavy chain constant region comprises Q347R, D399V and F405T substitutions relative to SEQ ID NO:118, numbered according to the EU numbering system.

55. The protein of claim 53, wherein one polypeptide chain of the antibody heavy chain constant region comprises an F405L substitution relative to SEQ ID NO:118; and the other polypeptide chain of the antibody heavy chain constant region comprises a K409R substitution relative to SEQ ID NO:118, numbered according to the EU numbering system.

56. The protein of any one of claims 53-55, wherein one polypeptide chain of the antibody heavy chain constant region comprises a Y349C substitution relative to SEQ ID NO:118; and the other polypeptide chain of the antibody heavy chain constant region comprises an S354C substitution relative to SEQ ID NO:118, numbered according to the EU numbering system.

57. A protein comprising:
(a) a first polypeptide comprising the amino acid sequence of SEQ ID NO:270;
(b) a second polypeptide comprising the amino acid sequence of SEQ ID NO:194; and
(c) a third polypeptide comprising the amino acid sequence of SEQ ID NO:195.

58. A protein comprising:
(a) a first polypeptide comprising the amino acid sequence of SEQ ID NO:271;
(b) a second polypeptide comprising the amino acid sequence of SEQ ID NO:272; and
(c) a third polypeptide comprising the amino acid sequence of SEQ ID NO:273.

59. A pharmaceutical composition comprising a protein according to any one of claims 1 to 58 and a pharmaceutically acceptable carrier.

60. A cell comprising one or more nucleic acids encoding a protein according to any one of claims 1 to 58.

61. A method of enhancing tumor cell death, the method comprising exposing the tumor cell and a natural killer cell to an effective amount of the protein of any one of claims 1 to 58 or the pharmaceutical composition of claim 59.

62. A method of treating cancer, the method comprising administering to a subject in need thereof an effective amount of the protein of any one of claims 1 to 58 or the pharmaceutical composition of claim 59.

63. The method according to claim 62, wherein the cancer is selected from the group consisting of B-cell non-Hodgkin's lymphoma (B-NHL), chronic lymphocytic leukemia (CLL), mantle cell lymphoma (MCL), follicular lymphoma (FL), diffuse large B-cell lymphoma (DLBCL), marginal zone lymphoma, mucosa-associated lymphoid tissue (MALT) lymphoma, primary mediastinal B-cell lymphoma, and acute lymphocytic leukemia (ALL).

64. A method of enhancing B cell death, the method comprising exposing the B cell and a natural killer cell to an effective amount of the protein of any one of claims 1 to 58 or the pharmaceutical composition of claim 59.

65. A method of treating an autoimmune inflammatory disease, the method comprising administering to a subject in need thereof an effective amount of the protein of any one of claims 1 to 58 or the pharmaceutical composition of claim 59.

66. A protein of any one of claims 1-58, wherein the protein is a purified protein.

67. The protein of claim 66, wherein the protein is purified using a method selected from the group consisting of: centrifugation, depth filtration, cell lysis, homogenization, freeze-thawing, affinity purification, gel filtration, ion exchange chromatography, hydrophobic interaction exchange chromatography, and mixed-mode chromatography.
